SEQUENCE LISTING (1 sequence)

SEQ ID NO: 1 - protein, 13 aa, synthetic construct
  SITE 2: note = X is a homocysteine contributing to a cystathionine unit (linking the homocysteine at residue 2 and cysteine at residue 10 via a thioether bond)
  DISULFID 1..6: note = Intrachain
  DISULFID 5..13: note = Intrachain
  CROSSLNK 2..10: note = homocysteine at position 2 and the cysteine at position 10 are connected by an internal sulfide (or thioether bond)
  SITE 1: note = Optionally acetylated N-terminus
CXELCCNVACYGC

(19) United States
(12) Patent Application Publication
(10) Pub. No.: US 2025/0320250 A1
(43) Pub. Date: Oct. 16, 2025

(54) PHARMACEUTICAL COMPOSITIONS FOR THE TREATMENT OF VISCERAL PAIN

(71) Applicant: Ironwood Pharmaceuticals, Inc., Boston, MA (US)

(72) Inventors: Michael GERBER, Boston, MA (US); Carolyn HIGGINS, Boston, MA (US)

(21) Appl. No.: 18/711,317

(22) PCT Filed: Nov. 28, 2022
(86) PCT No.: PCT/US2022/080490

§ 371 Date: May 17, 2024

Related U.S. Application Data
(60) Provisional application No. 63/283,731, filed on Nov. 29, 2021.
(60) Provisional application No. 63/382,612, filed on Nov. 7, 2022.

(51) Int. Cl. C07K 7/08; A61K 9/00; A61K 9/08; A61K 9/12; A61K 38/00; A61K 47/02; A61P 29/00

(52) U.S. Cl. CPC C07K 7/08; A61K 9/0031; A61K 9/08; A61K 9/122; A61K 47/02; A61P 29/00; A61K 38/00

(57) ABSTRACT

The present disclosure provides peptides, pharmaceutical compositions, liquid pharmaceutical compositions; pharmaceutical rectal foam compositions; and methods of making and using the same, that can be used for the treatment of a visceral pain condition (e.g., bladder pain associated with interstitial cystitis/bladder pain syndrome (IC/BPS).

### CROSS REFERENCE TO RELATED APPLICATIONS

The present application is a national phase application of PCT/US2022/080490, filed Nov. 28, 2022, which claims priority to and the benefit of U.S. Provisional Application No. 63/283,731, filed Nov. 29, 2021, and U.S. Provisional Application No. 63/382,612, filed Nov. 7, 2022, the contents of which are herein incorporated by reference in their entireties.

### SEQUENCE LISTING

This application incorporates by reference in its entirety the Sequence Listing XML entitled “223355-513525.xml” (3,810 bytes), which was created Aug. 26, 2022 at 3:09 PM, and filed electronically herewith.

### TECHNICAL FIELD

New pharmaceutical compositions, liquid pharmaceutical compositions; pharmaceutical rectal foam compositions; enemas; new processes, production techniques, new peptides, new formulations, and new combinations thereof, for the treatment of one or more visceral pain conditions, e.g., bladder pain associated with interstitial cystitis/bladder pain syndrome (IC/BPS), are described and claimed.

### BACKGROUND

Visceral pain conditions of the abdominal region include, e.g., bladder pain associated with interstitial cystitis/bladder pain syndrome (IC/BPS); pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology; affect more than 20% of the world's population. See Grundy et al., Visceral Pain. Annu Rev Physiol. 2019 Feb. 10; 81:261-284. However, despite its prevalence, visceral pain remains poorly understood.

Interstitial cystitis/bladder pain syndrome (IC/BPS) is a chronic condition involving bladder pain usually accompanied by urinary urgency, increased frequency, and/or nocturia. IC/BPS is often misdiagnosed as a urinary tract infection and antibiotics are generally ineffective. It is estimated that 3-7% of women and 3-4% of men meet the definition of IC/BPS. There may be several contributing factors for the cause of IC/BPS, and it is unknown if IC/BPS is a primary disorder or the secondary result of another disorder. See Hanno et al., Diagnosis and treatment of interstitial cystitis/bladder pain syndrome: AUA guideline amendment. Urol. 2015 May; 193 (5): 1545-53.

There are no diagnostic tests for IC/BPS, and diagnosis is generally based on urinary symptoms of urgency and frequency accompanied by pain related to the bladder. Diagnosis is generally reserved until other diseases that could cause these symptoms are ruled out.

There are few approved therapies available for IC/BPS. Patients often begin treatment with non-pharmacological treatments (general relaxation, stress management, behavior modification, and physical therapy techniques). Due to the marginally effective therapies available for IC/BPS, many patients utilize off-label therapies including intravesical instillations (i.e. mixtures of medications delivered directly to the bladder through a catheter) to relieve their symptoms. A need exists for more effective, well tolerated treatments for IC/BPS.

A 13-amino-acid, guanylate cyclase C (GC-C) agonist synthetic peptide is being developed for the treatment of bladder pain associated with IC/BPS and, potentially, other visceral pain conditions in the abdominal region.

Guanylate cyclase C (GC-C) is predominantly expressed on the luminal surface of the small and large intestines in the body. When GC-C receptors are stimulated, extracellular cyclic guanosine monophosphate (cGMP) is secreted across the basolateral membrane of colonic epithelial cells in the submucosa by multidrug resistance proteins MRP4 and MRP5, decreasing the activity of afferent nerve fibers located in the colonic wall, resulting in reduced visceral pain. This ultimately produces an analgesic effect in other organs in the abdominopelvic region via action mediated through the common afferent pathways. See Castro et al., Linaclotide Inhibits Colonic Nociceptors and Relieves Abdominal Pain via Guanylate Cyclase-C and Extracellular Cyclic GMP. Gastroenterology. 2013; 145(6):1334-46; and Grundy et al., Chronic linaclotide treatment reduces colitis-induced neuroplasticity and reverses persistent bladder dysfunction. JCI Insight. 2018; 3(19):e121841.

Experiments conducted in animals indicate that colonic hypersensitivity induces persistent hypersensitivity of bladder afferent pathways in the absence of bladder pathology. See Grundy et al., Chronic linaclotide treatment reduces colitis-induced neuroplasticity and reverses persistent bladder dysfunction. JCI Insight. 2018; 3(19). Hypersensitivity of bladder afferent pathways resembles the symptoms observed in patients with IC/BPS. Afferent neurons are known to have peripheral endings in both the colon and the bladder, and the axons of colonic and bladder neurons travel through the same splanchnic and pelvic nerves. These sensory afferents have cell bodies located within the thoracolumbar (TL) and lumbosacral (LS) dorsal root ganglia (DRG) and central projections in the dorsal horn of the corresponding regions of spinal cord. See Grundy et al., Cross-organ sensitization between the colon and bladder: to pee or not to pee? Am J Physiol Gastrointest Liver Physiol. 2018; 314:G301-G8.

Accordingly, new pharmaceutical compositions and methods to treat and/or reduce the symptoms associated with a visceral pain condition, e.g., IC/BPS, are needed.

### SUMMARY

The present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I):

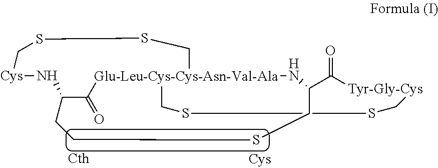

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.000498% w/w to about 0.335% w/w, of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount ranging from about 0.000498% w/w to about 0. 0.335% w/w, of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.000498% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00149% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00299% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00448% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00870% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00896% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00961% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.0124% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.0261% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.0288% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.301% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and an excipient; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is an amount that is about 0.335% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.000498% to about 0.335% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.000498% to about 0.335% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.000498% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00149% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00299% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00448% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00870% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00896% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00961% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0124% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0261% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0288% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.301% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.335% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In addition, the present disclosure describes a liquid pharmaceutical composition comprising a peptide, or a pharmaceutically acceptable salt thereof; and a plurality of excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the plurality of excipients comprise: 1.165 mg/mL of sodium phosphate monobasic monohydrate; 3.095 mg/mL of sodium phosphate dibasic heptahydrate; and an amount of water resulting in a total volume of the liquid pharmaceutical composition that is 20 mL; wherein the liquid pharmaceutical composition comprises an amount of peptide ranging from about 5 μg/mL to about 125 μg/mL; wherein the liquid composition has a pH ranging from about 6.9 to about 7.2.

In addition, the present disclosure describes a unit dosage form comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein said unit dosage form comprises an amount of the peptide ranging from about 100 μg to about 2500 μg.

In addition, the present disclosure describes an enema kit comprising a pharmaceutical composition comprising a peptide, or a pharmaceutically acceptable salt thereof; and one or more excipients, wherein the peptide comprises an amino acid sequence that is at least 80%, at least 85%, at least 90%, or at least 95% identical to an amino acid sequence according to Formula (I), wherein Cth is a cystathionine, wherein the cysteines at positions 1 and 6 (Cys₁ and Cys₆), and positions 5 and 13 (Cys₅ and Cys₁₃) are connected by disulfide bonds, and wherein cystathionine at position 2 (Cth₂) and the cysteine at position 10 (Cys₁₀) are connected by a thioether bond; a liquid composition comprising a peptide, or a pharmaceutically acceptable salt thereof; and one or more excipients, wherein the peptide comprises an amino acid sequence that is at least 80%, at least 85%, at least 90%, or at least 95% identical to an amino acid sequence according to Formula (I), wherein Cth is a cystathionine, wherein the cysteines at positions 1 and 6 (Cys₁ and Cys₆), and positions 5 and 13 (Cys₅ and Cys₁₃) are connected by disulfide bonds, and wherein cystathionine at position 2 (Cth₂) and the cysteine at position 10 (Cys₁₀) are connected by a thioether bond; or a unit dosage form comprising a peptide, or a pharmaceutically acceptable salt thereof; and one or more excipients, wherein the peptide comprises an amino acid sequence that is at least 80%, at least 85%, at least 90%, or at least 95% identical to an amino acid sequence according to Formula (I), wherein Cth is a cystathionine, wherein the cysteines at positions 1 and 6 (Cys₁ and Cys₆), and positions 5 and 13 (Cys₅ and Cys₁₃) are connected by disulfide bonds, and wherein cystathionine at position 2 (Cth₂) and the cysteine at position 10 (Cys₁₀) are connected by a thioether bond; a syringe, and an enema applicator.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I):

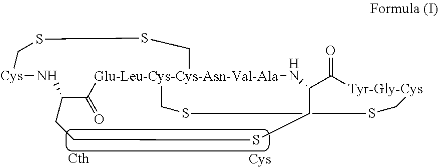

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.000498% to about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.000498% to about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.000498% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00149% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00299% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00448% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00870% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00896% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00961% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0124% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0261% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0288% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.301% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical composition comprising a peptide, or a pharmaceutically acceptable salt thereof; and one or more excipients; wherein the peptide comprises an amino acid sequence that is at least 80%, at least 85%, at least 90%, or at least 95% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In addition, the present disclosure describes a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

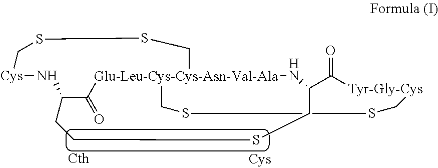

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In addition, the present disclosure describes a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

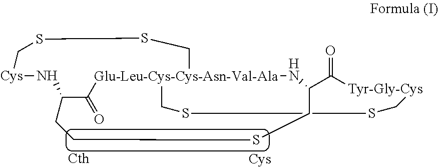

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0087% w/w of the of the total weight of the composition; and wherein the one or more excipients comprise: water in an amount that is about 69.8847% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In addition, the present disclosure describes a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

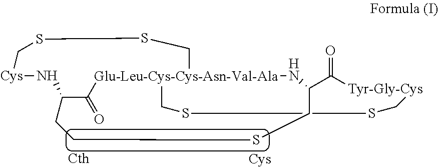

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0260% w/w of the of the total weight of the composition; and wherein the one or more excipients comprise: water in an amount that is about 69.8587% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In addition, the present disclosure describes a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

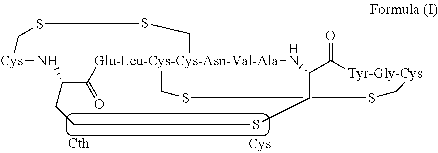

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0087% w/w of the of the total weight of the composition; and wherein the one or more excipients comprise: water in an amount that is about 69.8847% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In addition, the present disclosure describes a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

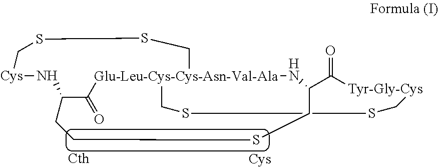

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0260% w/w of the of the total weight of the composition; and wherein the one or more excipients comprise: water in an amount that is about 69.8587% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In addition, the present disclosure describes a canister comprising the pharmaceutical rectal foam composition described herein.

In addition, the present disclosure describes a kit comprising the pharmaceutical rectal foam composition described herein, or the canister comprising the pharmaceutical rectal foam composition.

In addition, the present disclosure describes a method of treating a visceral pain condition in a subject in need thereof, the method comprising: administering to the subject in need thereof a therapeutically effective amount of the pharmaceutical composition described herein; the liquid pharmaceutical composition described herein; the unit dosage form described herein; or the pharmaceutical rectal foam composition described herein.

### BRIEF DESCRIPTION OF THE FIGURES

FIG. 1 shows a flow diagram illustrating the manufacturing process of the pharmaceutical composition of the present disclosure, formulated as a low-dose enema.

FIG. 2 shows a flow diagram illustrating the manufacturing process of the pharmaceutical composition of the present disclosure, formulated as a rectal foam.

FIG. 3 shows the method in which foam height of the pharmaceutical rectal foam composition was evaluated. Three separate actuations were performed using the apparatus shown here. Visual observation of the actuated foam was performed, and foam heights were averaged.

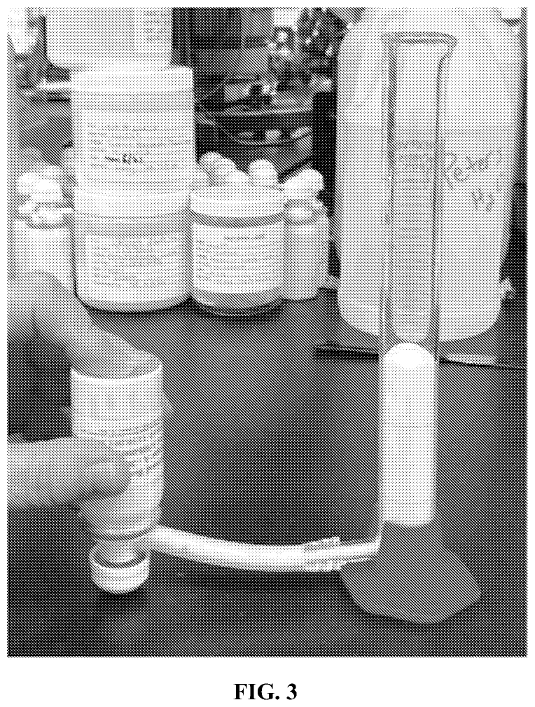

FIG. 4 depicts an illustration of the pharmaceutical rectal foam composition. Here, the liquid and vapor phase of propellant within the canister is portrayed in an inverted orientation.

FIG. 5 shows a flow diagram of the manufacturing process for the pharmaceutical rectal foam composition, 100 μg and 300 μg.

FIG. 6 shows package integrity for Prototype A, 30 μg and 3000 μg, and placebo, at 5° C. (left panel) and 25° C. (right panel) after 1-, 3-, and 6-months.

FIG. 7 shows package integrity for Prototype A, 30 μg and 3000 μg, and placebo, at 5° C. (left panel) and 25° C. (right panel) after 1-, 3-, and 6-months.

FIG. 8 shows the results of the foam collapse study. Here, the foams for placebo, Prototype A 30 μg/mL, and Prototype A 3000 μg/mL are shown at the initial time-point.

FIG. 9 shows the results of the foam collapse study. Here, the foams for placebo, Prototype A 30 μg/mL, and Prototype A 3000 μg/mL are shown after 15 minutes at 40° C.

FIG. 10 shows the results of the foam collapse study. Here, the foams for placebo, Prototype A 30 μg/mL, and Prototype A 3000 μg/mL are shown after 30 minutes at 40° C.

FIG. 11 shows the results of the foam collapse study. Here, the foams for placebo, Prototype A 30 μg/mL, and Prototype A 3000 μg/mL are shown after 45 minutes at 40° C.

FIG. 12 shows the results of the foam collapse study. Here, the foams for placebo, Prototype A 30 μg/mL, and Prototype A 3000 μg/mL are shown after 60 minutes at 40° C.

FIG. 13 shows the results of the foam collapse study. Here, the foams for placebo, Prototype B 30 μg/mL, and Prototype B 3000 μg/mL are shown at the initial time-point.

FIG. 14 shows the results of the foam collapse study. Here, the foams for placebo, Prototype B 30 μg/mL, and Prototype B 3000 μg/mL are shown after 15 minutes at 40° C.

FIG. 15 shows the results of the foam collapse study. Here, the foams for placebo, Prototype B 30 μg/mL, and Prototype B 3000 μg/mL are shown after 30 minutes at 40° C.

FIG. 16 shows the results of the foam collapse study. Here, the foams for placebo, Prototype B 30 μg/mL, and Prototype B 3000 μg/mL are shown after 45 minutes at 40° C.

FIG. 17 shows the results of the foam collapse study. Here, the foams for placebo, Prototype B 30 μg/mL, and Prototype B 3000 μg/mL are shown after 60 minutes at 40° C.

FIG. 18 depicts a schematic of a clinical study design. The study consists of 4 periods: a Screening Period, a Pretreatment Period, a 12-week Treatment Period, and a 2-week Follow-up Period.

FIG. 19 shows the Genitourinary Pain Index (GUPI) questionnaire for males.

FIG. 20 shows the Genitourinary Pain Index (GUPI) questionnaire for females.

FIG. 21 shows the Genitourinary Pain Index (GUPI) scoring guide in response to the questionnaire for males and females.

FIG. 22 shows the Global Response Assessment (GRA) scoring guide.

FIG. 23 shows the O'Leary/Sant Interstitial Cystitis Symptom Index (ICSI) questionnaire.

FIG. 24 shows the Margolis Body Map for Pain Assessment questionnaire.

FIG. 25 shows the Hospital Anxiety and Depression Scale (HADS) questionnaire.

FIG. 26 shows the EuroQoL Group EQ-5D questionnaire.

FIG. 27 shows the EuroQoL Group EQ-5D health scale questionnaire.

### DETAILED DESCRIPTION

### Definitions

Unless defined otherwise, all technical and scientific terms used herein have the same meaning as commonly understood by one of ordinary skill in the art to which this disclosure belongs. Accordingly, the following terms are intended to have the following meanings:

As used in the specification and claims, the singular form “a”, “an” and “the” includes plural references unless the context clearly dictates otherwise.

“Additive” refers to any pharmaceutically acceptable additive. Pharmaceutically acceptable additives include, without limitation, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes.

“ADLs” refers to activities of daily living.

“Administering” or “administration” or “administer” means to dispense, provide, and/or apply, and refers to any route of administration. For example, administering can refer to, e.g., administration as a suppository, parenteral, intraperitoneal, intramuscular, intralesional, intrathecal, intracranial, intranasal or subcutaneous administration, or the implantation of a slow-release device, e.g., a mini-osmotic pump, rectal foam, to a subject. Administration can be by any route, including parenteral and transmucosal (e.g., buccal, sublingual, palatal, gingival, nasal, vaginal, rectal, or transdermal). In some embodiments, parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a compound or composition described herein is administered at the same time, just prior to, or just after the administration of one or more additional agents or therapies, also referred to herein as a “additional agent.” The peptides of the present disclosure, or a pharmaceutical composition thereof, can be administered alone or can be co-administered to the patient. Co-administration is meant to include simultaneous or sequential administration of the compound individually or in combination (more than one compound or agent).

“AE” refers to adverse event

“Alignment” refers to a method of comparing two or more sequences (e.g., nucleotide, polynucleotide, amino acid, peptide, polypeptide or protein sequences) for the purpose of determining their relationship to each other. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22:4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5:113, 2004); Mafft, Kalign, ProbCons, and T-Coffee (see Notredame et al., T-Coffee: A novel method for multiple sequence alignments, Journal of Molecular Biology 302:205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In some embodiments, an alignment will introduce “phase shifts” and/or “gaps” into one or both of the sequences being compared in order to maximize the similarity between the two sequences, and scoring refers to the process of quantitatively expressing the relatedness of the aligned sequences.

“ALT” refers to alanine aminotransferase

“Ameliorate” or “amelioration” includes the arrest, prevention, decrease, or improvement in one or more the symptoms, signs, and features of the disease being treated, both temporary and long-term.

“AST” refers to aspartate aminotransferase.

“Binder” refers to” refers to any pharmaceutically acceptable binder that may be used in the practice of the invention. Examples of pharmaceutically acceptable binders include, without limitation, a starch (e.g., corn starch, potato starch and pre-gelatinized starch (e.g., STARCH 1500® and STARCH 1500 LM®, sold by Colorcon, Ltd.) and other starches), maltodextrin, gelatin, natural and synthetic gums such as acacia, powdered tragacanth, guar gum, cellulose and its derivatives (e.g., methylcellulose, hydroxyethyl cellulose, hydroxyethyl methylcellulose, hydroxypropyl cellulose and hydroxypropyl methylcellulose (hypromellose), ethyl cellulose, cellulose acetate, carboxymethyl cellulose calcium, sodium carboxymethyl cellulose, carboxymethylcellulose, microcrystalline cellulose (e.g. AVICEL™, such as, AVICEL-PH-101™, -103™ and -105™, sold by FMC Corporation, Marcus Hook, PA, USA)), polyvinyl alcohol, polyvinyl pyrrolidone (e.g., polyvinyl pyrrolidone K30), and mixtures thereof.

“BUN” refers to blood urea nitrogen.

“CFB” refers to change from baseline.

“CRP” refers to c-reactive protein.

“Combination” refers to simultaneous, separate, or sequential administration. For example, in some embodiments, “combination” refers to simultaneous administration. In another embodiment, “combination” refers to separate administration. In a further embodiments, of the invention “combination” refers to sequential administration. Where the administration is sequential or separate, the delay in administering the second component should not be such as to lose the beneficial effect of the combination.

“Cth” refers to cystathionine. Cystathionine possesses two α-amino carboxyl groups, designated “1” and “2” in Scheme 1 below, which can form peptide bonds:

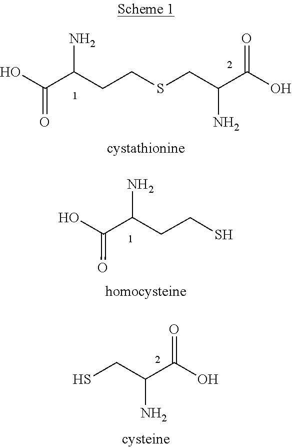 [SMILES: NC(CCSCC(N)C(=O)O)C(=O)O ; NC(CCS)C(=O)O ; NC(CS)C(=O)O]

To facilitate the use of the three letter amino acid code in describing the peptide of the present disclosure, when a cyclic peptide sequence is created by forming a peptide bond with each of the α-amino carboxyl group (designated “1” and “2”) at non-consecutive positions in the peptide sequence—which creates a cyclic thioether bridge—the peptide bond formed by the α-amino carboxyl group at “1” in Scheme 1 is designated “Cth,” whereas the peptide bond formed by the α-amino carboxyl group at “2” of Scheme 1 is designated “Cys.” See the section entitled “Synthetic Peptide” for further details. Homocysteine (Hcy) is shown above in Scheme 1. Those having ordinary skill in the art will recognize that cystathionine can be viewed as a combination of homocysteine and cysteine, wherein their side chains share a sulfur atom. Therefore, an alternative method of designating a cyclic peptide sequence created by forming a peptide bond with each of the α-amino carboxyl group of cystathionine at non-consecutive positions in the peptide sequence is by designating the peptide linkage formed by the α-amino carboxyl group at position 1 “Hcy” and the peptide linkage formed by the α-amino carboxyl group at position 2 “Cys.” Additional ways to designate the peptide of the present disclosure are provided in the sections below.

“Decreasing” or “decrease” or “decreased” or “reducing” or “reduced” or “a reduction” or “inhibiting” or any variation of these terms, refers to making something (e.g., the amount of pain) less in size, amount, intensity, or degree. For example, in some embodiments, the administration of a therapeutically effective amount of a peptide of the present disclosure, or a pharmaceutical composition thereof, to a patient in need thereof, results in the following effect: a decrease in the amount or level of pain in a patient who has been administered a therapeutically effective amount of a peptide of the present disclosure, or a pharmaceutical composition comprising the same; relative to the amount or level of pain experienced by the patient prior to being administered the therapeutically effective amount of a peptide of the present disclosure, or a pharmaceutical composition thereof. In some embodiments, reducing or decreasing, includes any measurable decrease or complete inhibition to achieve a desired result. For example, there may be a decrease of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, reduction of visceral pain compared to normal. About as used herein means within ±10%, preferably ±5% of a given value. Thus, in some embodiments, the terms “reduction in pain,” refers to a decrease or reduction in the amount of pain experienced by a patient who has received a therapeutically effective amount of a peptide of the present disclosure, or a pharmaceutical composition thereof, that is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 2.25%, at least about 2.5%, at least about 2.75%, at least about 3%, at least about 3.25%, at least about 3.5%, at least about 3.75%, at least about 4%, at least about 4.25%, at least about 4.5%, at least about 4.75%, at least about 5%, at least about 5.25%, at least about 5.5%, at least about 5.75%, at least about 6%, at least about 6.25%, at least about 6.5%, at least about 6.75%, at least about 7%, at least about 7.25%, at least about 7.5%, at least about 7.75%, at least about 8%, at least about 8.25%, at least about 8.5%, at least about 8.75%, at least about 9%, at least about 9.25%, at least about 9.5%, at least about 9.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, relative to the amount of pain experienced by the patient prior to being administered the therapeutically effective amount of a peptide of the present disclosure, or a pharmaceutical composition thereof.

“Disulfide bond” or “disulfide bridges” refers to a covalent bond between two cysteine residues, derived by the coupling of two thiol groups on their side chains. In some embodiments, a disulfide bond occurs via the oxidative folding of two different thiol groups (—SH) present in a polypeptide.

“eCRF” refers to electronic case report form.

“eDiary” refers to electronic diary.

“EQ-5D-5L” refers to EuroQol 5-Dimension 5-Level Version.

“Excipient” refers to any pharmaceutically acceptable additive, carrier, surfactant, emulsifier, thickener, preservative, solvent, disintegrant, glidant, lubricant, diluent, filler, bulking agent, binder, emollient, stiffening agent, chelating agent, stabilizer, solubilizing agents, dispersing agent, suspending agent, antioxidant, antiseptic, wetting agent, humectant, fragrant, suspending agents, pigments, colorants, isotonic agents, viscosity enhancing agents, mucoadhesive agents, and/or any combination thereof, that can be added to a pharmaceutical composition, preparation, and/or formulation, which may be useful in achieving a desired modification to the characteristics of the pharmaceutical composition, preparation, and/or formulation. Such modifications include, but are not limited to, physical stability, chemical stability, therapeutic efficacy, and/or any combination thereof.

“Filler” refers to any pharmaceutically acceptable filler that may be used in the practice of the invention. Examples of pharmaceutically acceptable fillers include, without limitation, talc, calcium carbonate (e.g., granules or powder), dibasic calcium phosphate, tribasic calcium phosphate, calcium sulfate (e.g., granules or powder), microcrystalline cellulose (e.g., Avicel PH101 or Celphere CP-305), powdered cellulose, dextrates, kaolin, mannitol, silicic acid, sorbitol, starch (e.g., Starch 1500), pre-gelatinized starch, lactose, glucose, fructose, galactose, trehalose, sucrose, maltose, isomalt, raffinose, maltitol, melezitose, stachyose, lactitol, palatinite, xylitol, myoinositol, and mixtures thereof.

“Hcy” refers to homocysteine.

“Homologous” or “homology” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. Homologous refers to the sequence similarity between two polypeptide molecules or between two nucleic acid molecules. The homology between two sequences is a function of the number of matching or homologous positions shared by the two sequences. For example, if 6 of 10 of the positions in two sequences are matched or homologous then the two sequences are 60% homologous. By way of example, the DNA sequences ATTGCC and TATGGC share 50% homology.

There may be partial homology, or complete homology and thus identical. “Sequence identity” refers to a measure of relatedness between two or more nucleic acids or two or more polypeptide sequences, and is given as a percentage with reference to the total comparison length. The identity calculation takes into account those nucleotide residues that are identical and in the same relative positions in their respective larger sequences.

“IBS-C” refers to irritable bowel syndrome with constipation.

“IBS-D” refers to irritable bowel syndrome with diarrhea.

“IC” refers to interstitial cystitis.

“IC/BPS” refers to interstitial cystitis/bladder pain syndrome.

“Identity” refers to a relationship between two or more polypeptide sequences or two or more polynucleotide sequences, as determined by comparing said sequences. The term “identity” also means the degree of sequence relatedness between two polypeptide or polynucleotide sequences, as the case may be, as determined by the match between amino acids or bases in the same position in the compared sequences. “Identity” and “similarity” can be readily calculated by any one of the myriad methods known to those having ordinary skill in the art, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994: Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988), the disclosures of which are incorporated herein by reference in their entireties. Furthermore, methods to determine identity and similarity are codified in publicly available computer programs. For example in some embodiments, methods to determine identity and similarity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), the disclosures of which are incorporated herein by reference in their entireties.

“Molecular weight (MW)” refers to the mass or weight of a molecule, and for proteins is typically measured in “daltons (Da)” or kilodaltons (kDa). In some embodiments, MW can be calculated using sodium dodecyl sulfate polyacrylamide gel electrophoresis (SDS-PAGE), gel chromatography, analytical ultracentrifugation, mass spectrometry, or light scattering. In some embodiments, the SDS-PAGE method is as follows: the sample of interest is separated on a gel with a set of molecular weight standards. The sample is run, and the gel is then processed with a desired stain, followed by destaining for about 2 to 14 hours. The next step is to determine the relative migration distance (Rf) of the standards and protein of interest. The migration distance can be determined using the following equation: Rf=(migration distance of the protein)/(Migration distance of the dye front). Next, the logarithm of the MW can be determined based on the values obtained for the bands in the standard; e.g., in some embodiments, the logarithm of the molecular weight of an SDS-denatured polypeptide and its relative migration distance (Rf) is plotted into a graph. After plotting the graph, interpolating the value derived will provide the molecular weight of the unknown protein band.

“One letter code” means the peptide sequence which is listed in its one letter code to distinguish the various amino acids in the primary structure of a protein: alanine=A, arginine=R, asparagine=N, aspartic acid=D, asparagine or aspartic acid=B, cysteine=C, glutamic acid=E, glutamine=Q, glutamine or glutamic acid=Z, glycine=G, histidine=H, isoleucine=I, leucine=L, lysine=K, methionine=M, phenylalanine=F, proline=P, serine=S, threonine=T, tryptophan=W, tyrosine=Y, and valine=V.

“Patient” and “subject” are used herein interchangeably, and refer to any animal (e.g., a mammal, such as a human, a laboratory animal, such as a mouse, rat, rabbit, guinea pig, or other animal models of visceral pain, or a domesticated animal, such as a dog, cat, or a domesticated animal, for example, sheep, horses, cattle, pigs and goats). A patient in need of treatment, according to the methods described herein, may be one who has been diagnosed with a visceral pain condition (e.g., bladder pain associated with interstitial cystitis/bladder pain syndrome (IC/BPS); pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology, diverticulitis pain, pain associated with gastrointestinal disorders, pain associated with venereal diseases, pain associated with irritable bowel syndrome (IBS), rectal pain, chronic proctalgia, proctalgia fugax, anal pain, chronic anal fissure, post-operative anal pain, pain associated with cancer, pain associated with gastrointestinal tract neoplasms, general pelvic pain, orchialgia, chronic prostatitis, prostatodynia, vulvodynia, urethral syndrome, penile pain, perianal pain, and pain associated with ulcerative colitis, ulcerative proctitis, or Crohn's disease), such as those described herein.

“Peptide” and “protein” and “polypeptide” are used interchangeably herein.

“Peptide of the present disclosure” refers to a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine residue; and wherein Cys₁ and Cys₆; and Cys₅ and Cys₁₃; are connected by disulfide bonds and Cth₂ and Cys₁₀ are connected by a thioether bridge (i.e. —CH₂CH₂SCH₂—). In some embodiments, the peptide of the present disclosure having an amino acid sequence set forth in SEQ ID NO: 1 can have an acetylated N-terminus; e.g., in some embodiments, the peptide of the present disclosure can be represented as follows: Ac-Cys₁-Cth₂-Glu₃-Leu₄-Cys₅-Cys₆-Asn₇-Val₈-Ala₉-Cys₁₀-Tyr₁₁-Gly₁₂-Cys₁₃-OH, wherein Cth is a cystathionine; wherein Cys₁ and Cys₆; Cys₅ and Cys₁₃; are connected by disulfide bonds; and wherein Cth₂ and Cys₁₀ are connected by a thioether bond; wherein Ac-indicates an acetylated N-terminus; and wherein-OH indicates an unmodified C-terminus.

In some embodiments, the peptide of the present disclosure having an amino acid sequence set forth in SEQ ID NO: 1 can be represented as having a homocysteine moiety in position 2 and the related des-sulfhydryl cysteine (or alanine) in position 10. For example, in some embodiments, the peptide of the present disclosure can be represented as follows: Ac-Cys₁-Hcy₂-Glu₃-Leu₄-Cys₅-Cys₆-Asn₇-Val₈-Ala₉-Ala₁₀-Tyr₁₁-Gly₁₂-Cys₁₃-OH [cyclo 1-6, 5-13; thioether 2-10]; wherein either of the foregoing peptides has a bond connectivity of Cys₁-Cys₆, Cys₅-Cys₁₃, Hcy₂-Ala₁₀; wherein Hcy is a homocysteine residue, wherein Ac-indicates an acetylated N-terminus; and wherein —OH indicates an unmodified C-terminus. The peptide of the present disclosure are discussed in greater detail below.

“Pharmaceutically acceptable salts” is meant to include salts of the peptide of present disclosure, which are prepared with relatively nontoxic acids or bases, depending on the particular substituents found on the compounds described herein. When peptides of the present disclosure contain relatively acidic functionalities, base addition salts can be obtained by contacting the neutral form of such peptides with a sufficient amount of the desired base, either neat or in a suitable inert solvent. Non-limiting examples of salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When peptides of the present disclosure contain relatively basic functionalities, acid addition salts can be obtained by contacting the neutral form of such peptides with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19).

“Pharmaceutical composition” or “pharmaceutical composition of the present disclosure” refers a pharmaceutical composition comprising a peptide of the present disclosure, and one or more excipients. For example, a pharmaceutical composition of the present disclosure refers to a pharmaceutical composition comprising an excipient, and a peptide of the present disclosure, a peptide having the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃; are connected by disulfide bonds; and wherein Cth₂ and Cys₁₀ are connected by a thioether bond. In some embodiments, a pharmaceutical composition of the present disclosure can be formulated in an enteral form; a parenteral form; or a transmucosal form. In other embodiments, a pharmaceutical composition of the present disclosure can be formulated in a suppository form, an enema form, a feeding tube form, or a solution for intraluminal use. In yet other embodiments, a pharmaceutical composition of the present disclosure can be formulated as an enema, a rectal gel, a rectal foam, a rectal aerosol, or a suppository. In such embodiments where the pharmaceutical composition of the present disclosure is formulated as a rectal foam, the terms “pharmaceutical composition” and “pharmaceutical rectal foam composition” are used interchangeably.

“QoL” refers to quality of life.

“SoA” refers to schedule of activities.

“Subject” or “patient” are used herein interchangeably, and refer to any animal (e.g., a mammal, such as a human) for whom diagnosis, prognosis, and/or treatment is desired. For example, in some embodiments, a subject can be a mammal, e.g., a human or non-human primate (such as an ape, monkey, orangutan, or chimpanzee), a dog, cat, guinea pig, rabbit, rat, mouse, horse, cattle, or cow. In certain embodiments, a “subject in need thereof” refers to one or more of the following: a subject diagnosed with a visceral pain condition and/or is exhibiting one or more conditions or symptoms associated with a visceral pain condition; a subject who has been diagnosed with or exhibited one or more conditions associated with a visceral pain condition in the past; or a subject who has been deemed at risk of developing a visceral pain condition or one or more conditions associated with a visceral pain condition in the future due to hereditary, lifestyle, and/or environmental factors.

“Therapeutically effective amount” or “effective amount” or “pharmaceutically effective amount” refer to a nontoxic but sufficient amount of one or more peptides described herein, or a pharmaceutically acceptable salt, hydrate or solvate thereof, or a pharmaceutical composition thereof, to provide the desired biological result, and/or to an amount sufficient to carry out a specifically stated purpose.

In some embodiments, the term “therapeutically effective amount” refers to an amount of a peptide of the present disclosure, or a pharmaceutical composition comprising the same, which is effective to “treat” a disease or condition (e.g., a visceral pain condition) in a subject (e.g., a mammal such as a human), and provides some improvement or benefit to a subject having the disease or condition (e.g., a visceral pain condition). Thus, a “therapeutically effective” amount is an amount that provides some alleviation, mitigation, and/or decrease in at least one clinical symptom of a visceral pain condition. Clinical symptoms associated with the diseases or conditions that can be treated by the methods of the disclosure are well known. Further, therapeutic effects need not be complete or curative, as long as some benefit is provided to the subject. In some embodiments, the term “therapeutically effective” refers to an amount of a therapeutic agent that is capable of alleviation or amelioration of one or more symptoms or conditions; diminishment of extent of condition, disorder or disease; stabilization of the state of condition, disorder or disease; prevention of development of condition, disorder or disease; prevention of spread of condition, disorder or disease; delay or slowing of condition, disorder or disease progression; delay or slowing of condition, disorder or disease onset; amelioration or palliation of the condition, disorder or disease state, and remission; limiting the symptoms of the condition, disorder or disease state; reducing the severity of the condition, disorder or disease state and/or any one or more symptoms associated thereof; relieving the pain associated with and/or caused by the condition, disorder or disease state; whether partial or total, in a subject in need thereof.

In some embodiments, a “therapeutically effective amount” can be determined empirically and in a routine manner, in relation to the stated purpose. And, an appropriate therapeutically effective amount in any individual case may be determined by one of ordinary skill in the art using routine experimentation. The actual amount administered and rate and time-course of administration, will depend on the nature and severity of what is being treated. Prescription of treatment, e.g., decisions on dosage etc., is within the responsibility of general practitioners and other medical doctors.

In some embodiments, a “therapeutically effective amount” can be an amount sufficient for a peptide of the present disclosure and/or a pharmaceutical composition comprising the same to accomplish a stated purpose relative to the absence of the peptide of the present disclosure and/or the pharmaceutical composition comprising the same (e.g., achieve the effect for which it is administered, treat a disease, reduce enzyme activity, increase enzyme activity, reduce a signaling pathway, and/or reduce one or more symptoms of a disease or condition). In one embodiment, an example of a “therapeutically effective amount” is an amount sufficient to contribute to the treatment, prevention, or reduction of a symptom or symptoms of a disease, condition, or symptom associated thereof (e.g., a visceral pain condition). In some embodiments, a “reduction” of a symptom or symptoms (and grammatical equivalents of this phrase) means decreasing of the severity or frequency of the symptom(s), or elimination of the symptom(s), either in whole or in part.

In some embodiments, a “therapeutically effective amount” can be an amount that has a prophylactic effect, e.g., an amount of a peptide of the present disclosure and/or a pharmaceutical composition comprising the same that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or one or more symptoms associated thereof. The full prophylactic effect does not necessarily occur by administration of one dose, and may occur only after administration of a series of doses. Thus, a prophylactically effective amount may be administered in one or more administrations.

In some embodiments, a “therapeutically effective amount” can be an amount that results in a decrease in activity (e.g., an “activity decreasing amount”). In some embodiments, an activity decreasing amount can be an amount of a peptide of the present disclosure and/or a pharmaceutical composition comprising the same that, when administered to a subject, decreases the activity of an enzyme relative to the absence of the peptide of the present disclosure and/or the pharmaceutical composition comprising the same.

In some embodiments, a “therapeutically effective amount” can be an amount that results in an increase in activity (e.g., an “activity increasing amount”). In some embodiments, an activity decreasing amount can be an amount of a peptide of the present disclosure and/or a pharmaceutical composition comprising the same that, when administered to a subject, increases the activity of an enzyme relative to the absence of the peptide of the present disclosure and/or the pharmaceutical composition comprising the same.

“Treatment” or “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions; diminishment of extent of condition, disorder or disease; stabilization of the state of condition, disorder or disease; prevention of development of condition, disorder or disease; prevention of spread of condition, disorder or disease; delay or slowing of condition, disorder or disease progression; delay or slowing of condition, disorder or disease onset; amelioration or palliation of the condition, disorder or disease state, and remission; limiting the symptoms of the condition, disorder or disease state; reducing the severity of the condition, disorder or disease state and/or any one or more symptoms associated thereof; relieving the pain associated with and/or caused by the condition, disorder or disease state; whether partial or total.

“Treating” or “reducing” or “inhibiting” or “limiting” or any variation of these terms, refers to making something (e.g., the number of symptoms, severity of symptoms, and/or frequency of symptoms, such as degree/severity of pain and/or frequency of pain) less in size, amount, intensity, or degree. For example, in some embodiments, the administration of a therapeutically effective amount of a peptide of the present disclosure and/or a pharmaceutical composition of the present disclosure, to a subject in need thereof, results in the following effect: a decrease in the frequency and/or severity of bladder pain associated with interstitial cystitis/bladder pain syndrome (IC/BPS); a decrease in the frequency and/or severity of urinary urgency associated with IC/BPS; a decrease in the frequency and/or severity of urinary frequency associated with IC/BPS; a decrease in the frequency and/or severity of nighttime voiding (nocturia) associated with IC/BPS; a decrease in the frequency and/or severity of burning sensation in the bladder associated with IC/BPS; a decrease in the frequency and/or severity of a burning sensation during urination associated with IC/BPS; a decrease in the frequency and/or severity of a pressure sensation in the bladder associated with IC/BPS; a decrease in the frequency and/or severity of discomfort in the bladder associated with IC/BPS; a decrease in the frequency and/or severity of bladder pain associated with IC/BPS; a decrease in the frequency and/or severity of urinary pain associated with IC/BPS; a decrease in the frequency and/or severity of genital pain associated with IC/BPS; a decrease in the frequency and/or severity of genitourinary pain associated with IC/BPS; a decrease in the frequency and/or severity of sleeping difficulties associated with IC/BPS; a decrease in the frequency and/or severity of body pain associated with IC/BPS; an increased quality of life associated with IC/BPS; a decrease in the frequency and/or severity of suicidal ideation that results from IC/BPS; a decrease in the frequency and/or severity of depression that results from IC/BPS; a reduction in the use of pain medication used by a subject to treat IC/BPS; a decrease in the frequency and/or severity of sexual dysfunction associated with IC/BPS; a decrease in the frequency and/or severity of loss of libido associated with IC/BPS; an increase in the ability to have sexual intercourse for a subject that previously did not have that ability because of one or more symptoms associated with IC/BPS; a decrease in the frequency and/or severity of bladder inflammation associated with IC/BPS; a decrease in the frequency and/or severity of Hunner's lesions (mucosal lesions or ulcerations seen with or without hydrodistension of the bladder) associated with IC/BPS; a decrease in the frequency and/or severity of pain of the abdominal region; a decrease in the frequency and/or severity of hypersensitivity of the bladder; a decrease in the frequency and/or severity of colonic pain; a decrease in the frequency and/or severity of extra-intestinal chronic pelvic pain; a decrease in the frequency and/or severity of endometriosis; a decrease in the frequency and/or severity of pain from menstrual cramps; a decrease in the frequency and/or severity of pain associated with endometriosis; a decrease in the frequency and/or severity of pain associated with intercourse; a decrease in the frequency and/or severity of pain during intercourse; a decrease in the frequency and/or severity of symptoms and/or pain associated with radiation proctopathy; a decrease in the frequency and/or severity of pain associated with vaginal irritation; a decrease in the frequency and/or severity of symptoms and/or pain associated with allodynia; a decrease in the frequency and/or severity of hypersensitivity of bladder afferent pathways in the absence of bladder pathology; a decrease in the frequency and/or severity of diverticulitis pain; a decrease in the frequency and/or severity of pain associated with gastrointestinal disorders; a decrease in the frequency and/or severity of pain associated with venereal diseases; a decrease in the frequency and/or severity of pain associated with irritable bowel syndrome (IBS); a decrease in the frequency and/or severity of rectal pain; a decrease in the frequency and/or severity of chronic proctalgia; a decrease in the frequency and/or severity of proctalgia fugax; a decrease in the frequency and/or severity of anal pain; a decrease in the frequency and/or severity of chronic anal fissure; a decrease in the frequency and/or severity of post-operative anal pain; a decrease in the frequency and/or severity of pain associated with cancer; a decrease in the frequency and/or severity of pain associated with gastrointestinal tract neoplasms; a decrease in the frequency and/or severity of general pelvic pain; a decrease in the frequency and/or severity of symptoms and/or pain associated with orchialgia; a decrease in the frequency and/or severity of chronic prostatitis; a decrease in the frequency and/or severity of prostatodynia; a decrease in the frequency and/or severity of vulvodynia; a decrease in the frequency and/or severity of urethral syndrome; a decrease in the frequency and/or severity of penile pain; a decrease in the frequency and/or severity of perianal pain; a decrease in the frequency and/or severity of pain associated with ulcerative colitis; a decrease in the frequency and/or severity of ulcerative proctitis; a decrease in the frequency and/or severity of symptoms and/or pain associated with Crohn's disease; or any combination thereof; relative to the number, frequency, and/or severity of these foregoing symptoms and/or pains in the subject prior to having been administered the therapeutically effective amount of a peptide of the present disclosure and/or a pharmaceutical composition of the present disclosure.

In some embodiments, limiting the symptoms of, reducing the severity of, or treating a visceral pain condition, includes any measurable decrease or complete inhibition to achieve a desired result. For example, there may be a decrease of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, in the number of symptoms, severity of symptoms, and/or frequency of symptoms (e.g., number, severity, and/or frequency of symptoms and/or pain associated with a visceral pain condition). About as used herein means within ±10%, preferably ±5% of a given value.

Thus, in some embodiments, the terms “limiting the symptoms of,” or “reducing the severity of,” or “treating a visceral pain condition,” refers to: a decrease or reduction in the frequency and/or severity of a symptom and/or pain associated with a visceral pain condition, when a therapeutically effective amount of a peptide of the present disclosure and/or a pharmaceutical composition of the present disclosure are administered to a subject in need thereof, that is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 2.25%, at least about 2.5%, at least about 2.75%, at least about 3%, at least about 3.25%, at least about 3.5%, at least about 3.75%, at least about 4%, at least about 4.25%, at least about 4.5%, at least about 4.75%, at least about 5%, at least about 5.25%, at least about 5.5%, at least about 5.75%, at least about 6%, at least about 6.25%, at least about 6.5%, at least about 6.75%, at least about 7%, at least about 7.25%, at least about 7.5%, at least about 7.75%, at least about 8%, at least about 8.25%, at least about 8.5%, at least about 8.75%, at least about 9%, at least about 9.25%, at least about 9.5%, at least about 9.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, relative to the frequency and/or severity of a symptom and/or a pain associated with a visceral pain condition experienced by the subject prior to receiving the therapeutically effective amount of a peptide of the present disclosure and/or a pharmaceutical composition of the present disclosure.

In some embodiments, “treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition. Thus, in some embodiments, treatment can refer to a 1%, 2%, 3%, 4%, 5%, 6%, 7%, 8%, 9%, 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 1%, 2%, 3%, 4%, 5%, 6%, 7%, 8%, 9%, 10%, 20%, 30%, 40%, 50%, 60%, 70%, 80%, 90% or greater as compared to a control level and such terms can include but do not necessarily include complete elimination.

“Thioether bond” refers to a covalent bond between the sidechain of a homocysteine and a cysteine residues in which the homocysteine and the cysteine sidechains share a sulfur atom. “Thioether bridge” refers to the bridge that is formed when a homocysteine sidechain and a cysteine sidechain are connected by a thioether bond and can be represented by —CH₂—CH₂—S—CH₂—.

“Unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Peptides of the present disclosure, and pharmaceutical compositions thereof, can be packaged in single or multiple unit dosage form for ease of administration and uniformity of dosage. The unit dosage form may be for a single daily dose or one of multiple daily doses (e.g., about 1 to 4 or more times per day). When multiple daily doses are used, the unit dosage form may be the same or different for each dose.

“Visceral pain condition” refers to one or more of the following: pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology; diverticulitis pain; pain associated with gastrointestinal disorders; pain associated with venereal diseases; pain associated with irritable bowel syndrome (IBS); rectal pain; chronic proctalgia; proctalgia fugax; anal pain; chronic anal fissure; post-operative anal pain; pain associated with cancer; pain associated with gastrointestinal tract neoplasms; general pelvic pain; orchialgia; chronic prostatitis; prostatodynia; vulvodynia; urethral syndrome; penile pain; perianal pain; and pain associated with ulcerative colitis; ulcerative proctitis; Crohn's disease; interstitial cystitis/bladder pain syndrome (IC/BPS) and/or any pain or symptom associated with IC/BPS, e.g., without limitation: a bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; or any combination thereof.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, and formulation. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000); each of these references are incorporated herein by reference in their entireties.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

All patent applications, patents, and printed publications referred to herein are incorporated by reference in their entirety to the same extent as if each individual publication, patent, or patent application was specifically and individually indicated to be incorporated by reference in its entirety. And, all patent applications, patents, and printed publications cited herein are incorporated herein by reference in the entireties, except for any definitions, subject matter disclaimers, or disavowals, and except to the extent that the incorporated material is inconsistent with the express disclosure herein, in which case the language in this disclosure controls.

### The Peptide of the Present Disclosure

The present disclosure contemplates peptides and pharmaceutical compositions comprising the same, that can be used for the treatment of a visceral pain condition (e.g., bladder pain associated with interstitial cystitis/bladder pain syndrome (IC/BPS); pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology, diverticulitis pain, pain associated with gastrointestinal disorders, pain associated with venereal diseases, pain associated with irritable bowel syndrome (IBS), rectal pain, chronic proctalgia, proctalgia fugax, anal pain, chronic anal fissure, post-operative anal pain, pain associated with cancer, pain associated with gastrointestinal tract neoplasms, general pelvic pain, orchialgia, chronic prostatitis, prostatodynia, vulvodynia, urethral syndrome, penile pain, perianal pain, and pain associated with ulcerative colitis, ulcerative proctitis, or Crohn's disease), such as those described herein.

In some embodiments, a peptide of the present disclosure is a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence set forth in Formula (1):

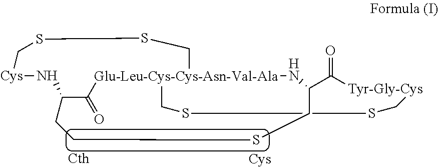

wherein Cth is a cystathionine.

Cystathionine, which has two α-amino carboxyl groups, designated “1” and “2” in Scheme 1, which can form peptide bonds, as shown below:

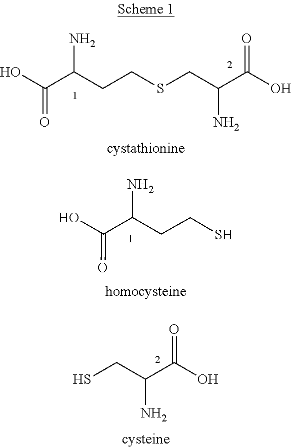 [SMILES: NC(CCSCC(N)C(=O)O)C(=O)O ; NC(CCS)C(=O)O ; NC(CS)C(=O)O]

However, to facilitate the use of the three letter amino acid code in describing a peptide sequence, when a cyclic peptide sequence is created by forming a peptide bond with each of the α-amino carboxyl group (designated “1” and “2”) at non-consecutive positions in the peptide sequence which creates a cyclic thioether bridge, the peptide bond formed by the α-amino carboxyl group at position 1 is designated “Cth,” whereas the peptide bond formed by the α-amino carboxyl group at position 2 is designated “Cys.”

As used herein, “Hcy” represents homocysteine as shown in Scheme 1. As shown in Scheme 1, cystathionine can be viewed as a combination of homocysteine and cysteine, wherein their side chains share a sulfur atom. Therefore, an alternative method of designating a cyclic peptide sequence created by forming a peptide bond with each of the α-amino carboxyl group of cystathionine at non-consecutive positions in the peptide sequence is by designating the peptide linkage formed by the α-amino carboxyl group at position “1” of Scheme 1 as “Hcy” and the peptide linkage formed by the α-amino carboxyl group at position “2” of Scheme 1 “Cys.”

Thus, it will be readily apparently to those having ordinary skill in the art that a peptide having an amino acid as set forth in Formula (I), comprises four cysteine residues at positions 1, 5, 6, and 13 (i.e., Cys₁; Cys₅; Cys₆; and Cys₁₃), and that these four cysteine residues form two disulfide bonds. Likewise, it will recognized by those having ordinary skill in the art that, as shown in Formula (I) above, a peptide bond is formed, between each of the α-amino carboxyl group (designated “1” and “2” in Scheme 1) at non-consecutive positions in the peptide sequence, which creates a cyclic thioether bridge.

Accordingly, to facilitate the use of the three letter amino acid code in describing a peptide sequence, when a cyclic peptide sequence is created by forming a peptide bond with each of the α-amino carboxyl group (designated “1” and “2”) at non-consecutive positions in the peptide sequence which creates a cyclic thioether bridge, the peptide bond formed by the α-amino carboxyl group at position 1 is designated “Cth,” whereas the peptide bond formed by the α-amino carboxyl group at position 2 is designated “Cys.”

Thus, one having ordinary skill in the art will recognize that, when describing the peptide of the present disclosure, either as a linear representation of the peptide, or using a structural formula, the cystathionine residue at position 2 can alternatively be represented as a homocysteine (Hcy) moiety in position 2 (Hcy₂). And, as those having ordinary skill in the art will recognize, the related des-sulfhydryl cysteine at position 10 (Cys₁₀), due to its chemical structure, can alternatively be represented as an alanine at position 10 (Ala₁₀). Additional descriptions of the linear representations of the peptide of the present disclosure are described herein.

The peptide of the present disclosure can be represented using a variety of alternative chemical structures known to those in the art. For example, an alternative way of showing the chemical structure for the peptide of the present disclosure is provided below, in Formula (II):

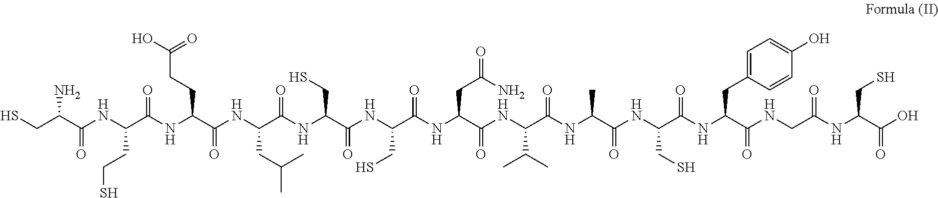 [SMILES: CC(C)C[C@H](NC(=O)[C@H](CCC(=O)O)NC(=O)[C@H](CCS)NC(=O)[C@@H](N)CS)C(=O)N[C@@H](CS)C(=O)N[C@@H](CS)C(=O)N[C@@H](CC(N)=O)C(=O)N[C@H](C(=O)N[C@@H](C)C(=O)N[C@@H](CS)C(=O)N[C@@H](Cc1ccc(O)cc1)C(=O)NCC(=O)N[C@@H](CS)C(=O)O)C(C)C]

wherein the cysteine residues at positions 1 and 6, and at 5 and 13 are linked by disulfide bonds; and wherein the cystathionine (Cth) unit at position 2, and the residue at position 10, are linked by an internal sulfide (or thioether) bond.

A further alternative method of representing the structural formula of the peptide of the present disclosure is shown below in Formula (III), wherein the peptide comprises four cysteine residues that form two disulfide bonds, and a cystathionine (Cth) unit (combining homocysteine and cysteine) providing an internal sulfide (or thioether) bond, with the defined connectivity shown in Formula (III), (i.e., Cys₁-Cys₆, Cys₅-Cys₁₃, Cth₂-Cys₁₀).

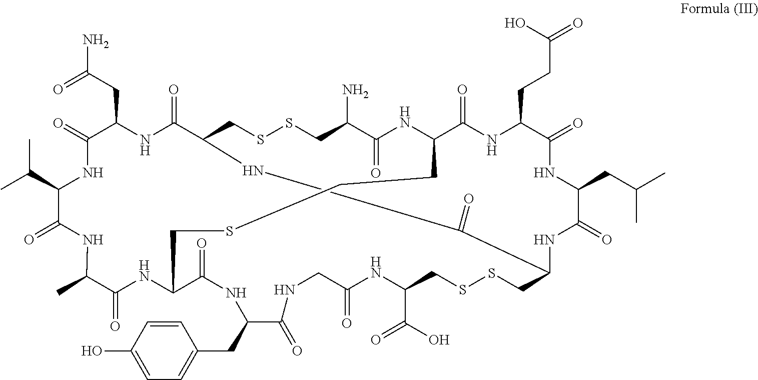 [SMILES: CC(C)C[C@@H]1NC(=O)[C@H](CCC(=O)O)NC(=O)[C@H]2CCSC[C@H]3NC(=O)[C@@H](C)NC(=O)[C@@H](C(C)C)NC(=O)[C@@H](CC(N)=O)NC(=O)[C@@H](CSSC[C@@H](N)C(=O)N2)NC(=O)[C@H](CSSC[C@@H](C(=O)O)NC(=O)CNC(=O)[C@@H](Cc2ccc(O)cc2)NC3=O)NC1=O]

Notwithstanding their different styles of representing the chemical structure of the peptide of the present disclosure, both Formula (I), Formula (II), and Formula (III) can be used interchangeably.

### Acetylated N-terminus

In some embodiments, the peptide of the present disclosure can have an acetylated N-terminus.

The chemical structure of the peptide of the present disclosure having an acetylated N-terminus can be represented using Formula (IV):

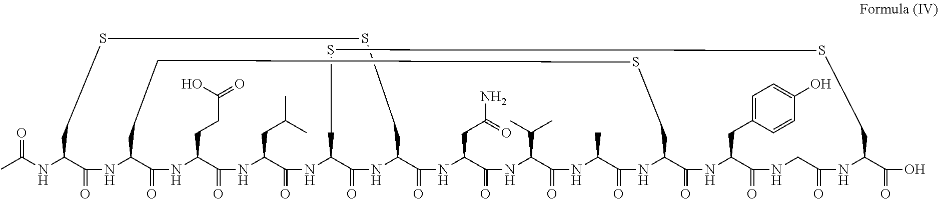 [SMILES: CC(=O)N[C@H]1CSSC[C@@H]2NC(=O)[C@@H]3CSSC[C@@H](C(=O)O)NC(=O)CNC(=O)[C@H](Cc4ccc(O)cc4)NC(=O)[C@H](CSCC[C@H](NC1=O)C(=O)N[C@@H](CCC(=O)O)C(=O)N[C@@H](CC(C)C)C(=O)N3)NC(=O)[C@H](C)NC(=O)[C@H](C(C)C)NC(=O)[C@H](CC(N)=O)NC2=O]

wherein the cysteine residues at positions 1, 5, 6, and 13 are linked by disulfide bonds; and wherein the cystathionine (Cth) unit at position 2, and the residue at position 10, are linked by an internal sulfide (or thioether) bond.

An alternative method of representing the structural formula of the peptide of the present disclosure having an acetylated N-terminus, is shown below in Formula (V), wherein the peptide comprises four cysteine residues that form two disulfide bonds, and a cystathionine (Cth) unit (combining homocysteine and cysteine) providing an internal sulfide (or thioether) bond, with the defined connectivity shown in Formula (IV), (i.e., Cys₁-Cys₆, Cys₅-Cys₁₃, Cth₂-Cys₁₀).

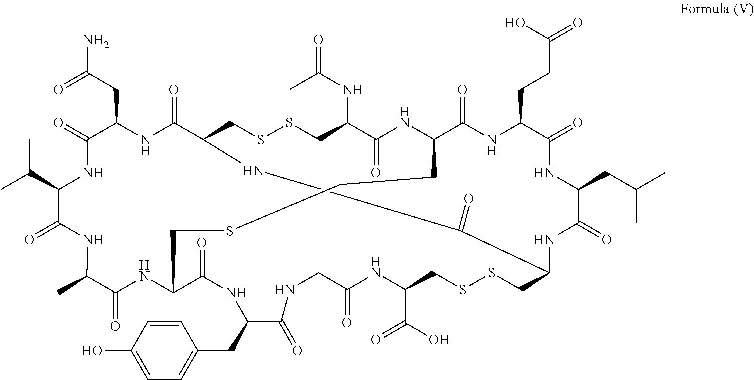 [SMILES: CC(=O)N[C@@H]1CSSC[C@H]2NC(=O)[C@@H]3CSSC[C@@H](C(=O)O)NC(=O)CNC(=O)[C@@H](Cc4ccc(O)cc4)NC(=O)[C@@H](CSCC[C@@H](NC1=O)C(=O)N[C@@H](CCC(=O)O)C(=O)N[C@@H](CC(C)C)C(=O)N3)NC(=O)[C@@H](C)NC(=O)[C@@H](C(C)C)NC(=O)[C@@H](CC(N)=O)NC2=O]

As described above, those having ordinary skill in the art will recognize there are alternative methods for describing the peptide of the present disclosure having an acetylated N-terminus.

For example, in addition to a chemical structure or linear representation, the peptide of the present disclosure having an acetylated N-terminus can be described as follows: N^{α}-acetyl-{L-hemicystinyl¹-[L-homocysteinyl²-L-glutamyl³-L-leucyl⁴-(L-hemicystinyl⁵-L-hemicystinyl⁶}-L-asparagyl⁷-L-valyl⁸-L-alanyl⁹-L-alanyl¹⁰]-L-tyrosyl¹¹-glycyl¹²-L-hemicystine¹³) acid, cyclic bis-disulfide 1-6, 5-13, thioether 2-10.

### Linear Representations of the Peptide of the Present Disclosure

Those having ordinary skill in the art will recognize that the peptide set forth in Formulas (I):

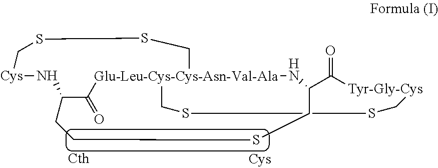

wherein Cth is a cystathionine; can be linearly represented in a variety of ways.

As described above, cystathionine (Cth), has two α-amino carboxyl groups, designated as positions “1” and “2” in Scheme 1 below, which form peptide bonds:

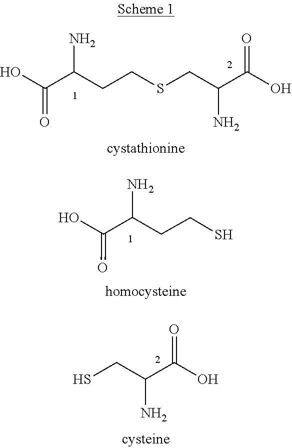 [SMILES: NC(CCSCC(N)C(=O)O)C(=O)O ; NC(CCS)C(=O)O ; NC(CS)C(=O)O]

However, to facilitate the use of the three letter amino acid code in describing a peptide sequence, when a cyclic peptide sequence is created by forming a peptide bond with each of the α-amino carboxyl group (designated “1” and “2”) at non-consecutive positions in the peptide sequence which creates a cyclic thioether bridge, the peptide bond formed by the α-amino carboxyl group at position 1 is designated “Cth,” whereas the peptide bond formed by the α-amino carboxyl group at position 2 is designated “Cys.”

Homocysteine (Hcy) is shown above in Scheme 1; cystathionine can be viewed as a combination of homocysteine and cysteine, wherein their side chains share a sulfur atom. Therefore, an alternative method of designating a cyclic peptide sequence created by forming a peptide bond with each of the α-amino carboxyl group of cystathionine at non-consecutive positions in the peptide sequence is by designating the peptide linkage formed by the α-amino carboxyl group at position “1” of Scheme 1 as “Hcy” and the peptide linkage formed by the α-amino carboxyl group at position “2” of Scheme 1 “Cys.”

Thus, it will be readily apparently to those having ordinary skill in the art that a peptide having an amino acid as set forth in Formula (I), comprises four cysteine residues at positions 1, 5, 6, and 13 (i.e., Cys₁; Cys₅; Cys₆; and Cys₁₃), and that these four cysteine residues form two disulfide bonds. Likewise, it will recognized by those having ordinary skill in the art that, as shown in Formula (I) above, a peptide bond is formed, between each of the α-amino carboxyl group (designated “1” and “2” in Scheme 1) at non-consecutive positions in the peptide sequence, which creates a cyclic thioether bridge.

Thus, one having ordinary skill in the art will recognize that, when describing the peptide of the present disclosure, either as a linear representation of the peptide, or using a structural formula, the use of Cth at position 2 of Formula (I) can alternatively be represented as a homocysteine (Hcy) moiety a position 2 (Hcy₂). And, as those having ordinary skill in the art will recognize that the related des-sulfhydryl cysteine at position 10 (Cys₁₀) of Formula (I), due to its chemical structure, can alternatively be represented as an alanine at position 10 (Ala₁₀).

While the linear representation of the peptide of the present disclosure having an amino acid sequence as set forth in Formula (I), shall be linearly represented using a three letter amino acid code, e.g., as follows: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine residue; and wherein Cys₁ and Cys₆; and Cys₅ and Cys₁₃; are connected by disulfide bonds; and Cth₂ and Cys₁₀ are connected by a thioether bond; additional descriptions of the linear representations of the peptide of the present disclosure are described herein.

For example, alternatively, in other embodiments, the peptide of the present disclosure can be linearly represented using the one letter amino acid code, e.g., as follows: CXELCCNVACYGC (SEQ ID NO: 1); wherein X is a cystathionine (Cth) residue; and wherein cysteines at positions 1 and 6; and 5 and 13 are connected by disulfide bonds; and the cystathionine at position 2 and the cysteine at position 10 are connected by a thioether bond.

Accordingly, the peptide of the present disclosure can be linearly represented using the following three- or one-letter amino acid codes, all of which are used interchangeably:
Cys₁ Cth₂ Glu₃ Leu₄ Cys₅ Cys₆ Asn₇ Val₈ Ala₉ Cys₁₀ Tyr₁₁ Gly₁₂ Cys₁₃; wherein Cth is a cystathionine; wherein Cys₁ and Cys₆; Cys₅ and Cys₁₃; are connected by disulfide bonds; and wherein Cth₂ and Cys₁₀ are connected by a thioether bond;
Cys₁ Hcy₂ Glu₃ Leu₄ Cys₅ Cys₆ Asn₇ Val₈ Ala₉ Ala₁₀ Tyr₁₁ Gly₁₂ Cys₁₃; wherein Hcy is a homocysteine; wherein Cys₁ and Cys₆; Cys₅ and Cys₁₃; are connected by disulfide bonds; and wherein Cth₂ and Cys₁₀ are connected by a thioether bond;
CXELCCNVACYGC; wherein X is a cystathionine; wherein the residues at positions 1 and 6, and 5 and 13 are connected by disulfide bonds; and wherein the residues at positions 2 and 10 are connected by a thioether bond; and
CXELCCNVACYGC; wherein X is a homocysteine; wherein the residues at positions 1 and 6, and 5 and 13 are connected by disulfide bonds; and wherein the residues at positions 2 and 10 are connected by a thioether bond.

In addition, in some embodiments, any of the foregoing linear representations of the peptide of the present disclosure can be described to show an N-terminus acetyl group (i.e. “Ac—)” and/or an unmodified C-terminus (e.g., “—COOH” or “—OH”). Thus, in some embodiments, a peptide of the present disclosure, having an acetylated N-terminus and an unmodified C-terminus, can be linearly represented using the following three- or one-letter amino acid codes, all of which are used interchangeably:
Ac-Cys₁ Cth₂ Glu₃ Leu₄ Cys₅ Cys₆ Asn₇ Val₈ Ala₉ Cys₁₀ Tyr₁₁ Gly₁₂ Cys₁₃-OH; wherein Cth is a cystathionine; wherein Cys₁ and Cys₆; Cys₅ and Cys₁₃; are connected by disulfide bonds; wherein Cth₂ and Cys₁₀ are connected by a thioether bond; wherein Ac-indicates an acetylated N-terminus; and wherein-OH indicates an unmodified C-terminus;
Ac-Cys₁ Hcy₂ Glu₃ Leu₄ Cys₅ Cys₆ Asn₇ Val₈ Ala₉ Ala₁₀ Tyr₁₁ Gly₁₂ Cys₁₃-OH; wherein Cth is a cystathionine; wherein Cys₁ and Cys₆; Cys₅ and Cys₁₃; are connected by disulfide bonds; wherein Cth₂ and Cys₁₀ are connected by a thioether bond; wherein Ac-indicates an acetylated N-terminus; and wherein-OH indicates an unmodified C-terminus
Ac-CXELCCNVACYGC-OH; wherein X is a cystathionine; wherein the residues at positions 1 and 6, and 5 and 13 are connected by disulfide bonds; wherein the residues at positions 2 and 10 are connected by a thioether bond; wherein Ac-indicates an acetylated N-terminus; and wherein-OH indicates an unmodified C-terminus
Ac-CXELCCNVACYGC-OH; wherein X is a homocysteine; wherein the residues at positions 1 and 6, and 5 and 13 are connected by disulfide bonds; wherein the residues at positions 2 and 10 are connected by a thioether bond; wherein Ac-indicates an acetylated N-terminus; and wherein-OH indicates an unmodified C-terminus.

In some embodiments, a peptide of the present disclosure can comprise, consist essentially of, or consist of, an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond.

In some embodiments, the peptide of the present disclosure can have an acetylated N-terminus.

### Making Peptides of the Present Disclosure

Methods of producing proteins are well known in the art, and there are a variety of techniques available. For example, in some embodiments, proteins can be produced using recombinant methods (e.g., a recombinant expression system).

In other embodiments, a peptide of the present disclosure can be chemically synthesized.

Synthetic peptides and methods regarding the same, can be performed by those having ordinary skill in the art, and/or through the use of commercial vendors (e.g., GenScript®; Piscataway, New Jersey). For example, in some embodiments, chemical peptide synthesis can be achieved using Liquid phase peptide synthesis (LPPS), or solid phase peptide synthesis (SPPS).

In some embodiments, peptide synthesis can generally be achieved by using a strategy wherein the coupling the carboxyl group of a subsequent amino acid to the N-terminus of a preceding amino acid generates the nascent polypeptide chain—a process that is opposite to the type of polypeptide synthesis that occurs in nature.

Exemplary methods of peptide synthesis can be found in Anderson G. W. and McGregor A. C. (1957) T-butyloxycarbonylamino acids and their use in peptide synthesis. Journal of the American Chemical Society. 79, 6180-3; Carpino L. A. (1957) Oxidative reactions of hydrazines. Iv. Elimination of nitrogen from 1, 1-disubstituted-2-arenesulfonhydrazides1-4. Journal of the American Chemical Society. 79, 4427-31; Mckay F. C. and Albertson N. F. (1957) New amine-masking groups for peptide synthesis. Journal of the American Chemical Society. 79, 4686-90; Merrifield R. B. (1963) Solid phase peptide synthesis. I. The synthesis of a tetrapeptide. Journal of the American Chemical Society. 85, 2149-54; Carpino L. A. and Han G. Y. (1972) 9-fluorenylmethoxycarbonyl amino-protecting group. The Journal of Organic Chemistry. 37, 3404-9; and A Lloyd-Williams P. et al. (1997) Chemical approaches to the synthesis of peptides and proteins. Boca Raton: CRC Press. 278; U.S. Pat. No. 3,714,140 (filed Mar. 16, 1971); U.S. Pat. No. 4,411,994 (filed Jun. 8, 1978); U.S. Pat. No. 7,785,832 (filed Jan. 20, 2006); U.S. Pat. No. 8,314,208 (filed Feb. 10, 2006); and U.S. Pat. No. 10,442,834 (filed Oct. 2, 2015); and United States Patent Application 2005/0165215 (filed Dec. 23, 2004), the disclosures of which are incorporated herein by reference in their entirety.

A method of making a peptide of formulas I-V can be found in U.S. Pat. No. 10,618,938, the disclosure of which is incorporated by reference in its entirety.

### Pharmaceutically Acceptable Salts

In some embodiments, pharmaceutically acceptable salts, hydrates, solvates, crystal forms and individual isomers, enantiomers, tautomers, diastereomers and prodrugs of the peptide described herein can be utilized.

In some embodiments, a pharmaceutically acceptable salt of the present disclosure possesses the desired pharmacological activity of the parent compound. Such salts include: acid addition salts, formed with inorganic acids; acid addition salts formed with organic acids; or salts formed when an acidic proton present in the parent compound is replaced by a metal ion, e.g., an alkali metal ion, aluminum ion; or coordinates with an organic base such as ethanolamine, and the like.

In some embodiments, pharmaceutically acceptable salts include conventional toxic or non-toxic salts. For example, in some embodiments, convention non-toxic salts include those such as fumarate, phosphate, citrate, chlorydrate, and the like. In some embodiments, the pharmaceutically acceptable salts of the present disclosure can be synthesized from a parent compound by conventional chemical methods. In some embodiments, such salts can be prepared by reacting the free acid or base forms of these compounds with a stoichiometric amount of the appropriate base or acid in water or in an organic solvent, or in a mixture of the two. In some embodiments, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is incorporated herein by reference in its entirety.

In some embodiments, a pharmaceutically acceptable salt can be one of the following: hydrochloride; sodium; sulfate; acetate; phosphate or diphosphate; chloride; potassium; maleate; calcium; citrate; mesylate; nitrate; tartrate; aluminum; or gluconate.

In some embodiments, a list of pharmaceutically acceptable acids that can be used to form salts can be: glycolic acid; hippuric acid; hydrobromic acid; hydrochloric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; nitric acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); undecylenic acid; a 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; or glycerophosphoric acid.

In some embodiments, pharmaceutically acceptable salt can be any organic or inorganic addition salt.

In some embodiments, the salt may use an inorganic acid and an organic acid as a free acid. The inorganic acid may be hydrochloric acid, bromic acid, nitric acid, sulfuric acid, perchloric acid, phosphoric acid, etc. The organic acid may be citric acid, acetic acid, lactic acid, maleic acid, fumaric acid, gluconic acid, methane sulfonic acid, gluconic acid, succinic acid, tartaric acid, galacturonic acid, embonic acid, glutamic acid, aspartic acid, oxalic acid, (D) or (L) malic acid, maleic acid, methane sulfonic acid, ethane sulfonic acid, 4-toluene sulfonic acid, salicylic acid, citric acid, benzoic acid, malonic acid, etc.

In some embodiments, the salts include alkali metal salts (sodium salts, potassium salts, etc.) and alkaline earth metal salts (calcium salts, magnesium salts, etc.). For example, the acid addition salt may include acetate, aspartate, benzoate, besylate, bicarbonate/carbonate, bisulfate/sulfate, borate, camsylate, citrate, edisilate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthalate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salt, etc., and among them, hydrochloride or trifluoroacetate may be used.

In yet other embodiments, the pharmaceutically acceptable salt can be a salt with an acid such as acetic acid, propionic acid, butyric acid, formic acid, trifluoroacetic acid, maleic acid, tartaric acid, citric acid, stearic acid, succinic acid, ethylsuccinic acid, lactobionic acid, gluconic acid, glucoheptonic acid, benzoic acid, methanesulfonic acid, ethanesulfonic acid, 2-hydroxyethanesulfonic acid, benzenesulfonic acid, p-toluenesulfonic acid, laurylsulfuric acid, malic acid, aspartic acid, glutaminic acid, adipic acid, cysteine, N-acetylcysteine, hydrochloric acid, hydrobromic acid, phosphoric acid, sulfuric acid, hydroiodic acid, nicotinic acid, oxalic acid, picric acid, thiocyanic acid, undecanoic acid, polyacrylate or carboxyvinyl polymer.

In some embodiments, the pharmaceutically acceptable salt can be prepared from either inorganic or organic bases. Salts derived from inorganic bases include, but are not limited to, the sodium, potassium, lithium, ammonium, calcium, magnesium, ferrous, zinc, copper, manganous, aluminum, ferric, manganic salts, and the like. Preferred inorganic salts are the ammonium, sodium, potassium, calcium, and magnesium salts. Salts derived from organic bases include, but are not limited to, salts of primary, secondary, and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. Preferred organic bases are isopropylamine, diethylamine, ethanolamine, piperidine, tromethamine, and choline.

In some embodiments, pharmaceutically acceptable salt refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66:1-19 (1977), the disclosure of which is incorporated herein by reference in its entirety.

In some embodiments, the salts of the present disclosure can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Exemplary descriptions of pharmaceutically acceptable salts is provided in P. H. Stahl and C. G. Wermuth, (editors), Handbook of Pharmaceutical Salts: Properties, Selection and Use, John Wiley & Sons, August 23, (2002), the disclosure of which is incorporated herein by reference in its entirety.

### Chromatographic Purity

Chromatographic purity of the peptides of the present disclosure may be assessed by performing HPLC under the conditions described herein. For example, in some embodiments, the area under the peptide peak is measured and compared to the total area under all peaks excluding the solvent peak and any non-polypeptide related peaks (i.e., peaks associated with excipients that may be observed in a placebo).

In some embodiments, the chromatographic purity of a peptide in a composition after storage at room temperature or accelerated conditions at a specified time point of storage under accelerated conditions [40° C./75% RH] or 12, 18, 24 or more months of storage under room temperature conditions [25° C./60% RH]) can be compared to the chromatographic purity of peptides in a composition at an initial time (e.g., the time when the pharmaceutical composition is released for clinical or patient use (“the release date”)) to provide the chromatographic purity value.

For example, the chromatographic purity of the peptide in a composition is measured after storage for a specified time at, e.g., accelerated conditions (40° C./75% RH) and compared to the chromatographic purity of peptide in the composition at the release date.

In some embodiments, the chromatographic purity of the peptide in a composition is measured after storage for a specified time at room temperature conditions (25° C./60% RH) and compared to the chromatographic purity of peptide in the composition at the release date.

### Pharmaceutical Compositions

As used herein, “v/v” or “% v/v” or “volume per volume” refers to the volume concentration of a solution (“v/v” stands for volume per volume). Here, v/v can be used when both components of a solution are liquids. For example, when 50 mL of ingredient X is diluted with 50 mL of water, there will be 50 mL of ingredient X in a total volume of 100 mL; therefore, this can be expressed as “ingredient X 50% v/v.” Percent volume per volume (% v/v) is calculated as follows: (volume of solute (mL)/volume of solution (100 mL)); e.g., % v/v=mL of solute/100 mL of solution.

As used herein, “w/w” or “% w/w” or “weight per weight” or “% wt/wt” refers to the weight concentration of a solution, i.e., percent weight in weight (“w/w” stands for weight per weight). Here, w/w expresses the number of grams (g) of a constituent in 100 g of solution or mixture. For example, a mixture consisting of 30 g of ingredient X, and 70 g of water would be expressed as “ingredient X 30% w/w.” Percent weight per weight (% w/w) is calculated as follows: (weight of solute (g)/weight of solution (g))×100; or (mass of solute (g)/mass of solution (g))×100.

As used herein, “w/v” or “% w/v” or “weight per volume” refers to the mass concentration of a solution, i.e., percent weight in volume (“w/v” stands for weight per volume). Here, w/v expresses the number of grams (g) of a constituent in 100 mL of solution. For example, if 1 g of ingredient X is used to make up a total volume of 100 mL, then a “1% w/v solution of ingredient X” has been made. Percent weight per volume (% w/v) is calculated as follows: (Mass of solute (g)/Volume of solution (mL))×100.

The present disclosure contemplates combinations, mixtures, and compositions comprising, consisting essentially of, or consisting of, any one or more of the peptides described herein. The preparation of a pharmaceutical composition that contains a peptide of the present disclosure will be known to those of skill in the art in light of the present disclosure, as exemplified by Remington's Pharmaceutical Sciences, 18th Ed. Mack Printing Company, 1990, Moreover, for animal (e.g., human) administration, it will be understood that preparations should meet sterility, pyrogenicity, general safety, and purity standards.

Formulations may be employed in admixtures with conventional excipients, i.e., pharmaceutically acceptable organic or inorganic carrier substances suitable for parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents. e.g., other analgesic agents.

In some embodiments, one or more of the peptides of the present disclosure may be administered as a pharmaceutical composition in which the one or more peptides are admixed with an appropriate pharmaceutically acceptable carrier, diluent, excipient, vehicle, or carrier.

In some embodiments, a pharmaceutical composition can comprise, consist essentially of, or consist of, a peptide, or a pharmaceutically acceptable salt, hydrate, or solvate thereof, in association with a pharmaceutically acceptable diluent or carrier.

In some embodiments, the pharmaceutical compositions of the present disclosure may be administered parenterally, or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. Methods of administration are described in detail below.

In some embodiments, the present disclosure provides a pharmaceutical composition comprising a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; and one or more excipients.

### Excipients

In some embodiments, a pharmaceutical composition of the present disclosure can comprise, consist essentially of, or consist of, a peptide of the present disclosure, and one or more excipients.

For example in some embodiments, an excipient can be pharmaceutically acceptable additive, carrier, surfactant, emulsifier, thickener, preservative, solvent, disintegrant, glidant, lubricant, diluent, filler, bulking agent, binder, emollient, stiffening agent, chelating agent, emulsifier, stabilizer, dispersing agent, suspending agent, antioxidant, antiseptic, and/or any combination thereof, that can be added to a pharmaceutical composition, preparation, and/or formulation, which may be useful in achieving a desired modification to the characteristics of the pharmaceutical composition, preparation, and/or formulation. Such modifications include, but are not limited to, physical stability, chemical stability, therapeutic efficacy, and/or any combination thereof.

In some embodiments, e.g., the excipient can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In other embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In some embodiments, each of the one or more pharmaceutically acceptable excipients can be independently selected from thickeners, viscosity enhancing agents, bulking agents, mucoadhesive agents, buffers, preservatives, and fillers.

In some embodiments, each of the one or more pharmaceutically acceptable excipients can be independently selected from diluents, binders, lubricants, glidants, and disintegrants.

### Carriers

In some embodiments, a pharmaceutical composition comprises a peptide of the present disclosure and a carrier.

In some embodiments, a pharmaceutical composition comprises a peptide of the present disclosure and a liquid carrier vehicle.

In some embodiments, a pharmaceutical composition comprises a peptide of the present disclosure and a liquid carrier vehicle, wherein the liquid carrier vehicle can be, by way of non-limiting example, purified water, propylene glycol, polyethyleneglycol, ethanol, 1-propanol, 2-propanol, 1-propen-3-ol (allyl alcohol), propylene glycol, glycerol, 2-methyl-2-propanol, formamide, methyl formamide, dimethyl formamide, ethyl formamide, diethyl formamide, acetamide, methyl acetamide, dimethyl acetamide, ethyl acetamide, diethyl acetamide, 2-pyrrolidone, N-methyl-2-pyrrolidone, N-ethyl-2-pyrrolidone, tetramethyl urea, 1,3-dimethyl-2-imidazolidinone, propylene carbonate, 1,2-butylene carbonate, 2,3-butylene carbonate, dimethyl sulfoxide, diethyl sulfoxide, hexamethyl phosphoramide, pyruvic aldehyde dimethylacetal, dimethylisosorbide and combinations thereof.

In some embodiments, a pharmaceutical composition of the present disclosure comprising a liquid carrier may contain an amount of liquid carrier ranging from about 0.005 wt % to about 99 wt %.

### Surfactants and Emulsifiers

In some embodiments, a pharmaceutical composition comprises a peptide of the present disclosure, and one or more surfactants and/or emulsifiers.

In some embodiments, a pharmaceutical composition comprises a peptide of the present disclosure, and one or more surfactants and/or emulsifiers, wherein the one or more surfactants and/or emulsifiers can include, by way of non-limiting example, mixtures of cetostearylic alcohol with sorbitan esterified with polyoxyethylenic fatty acids, polyoxyethylene fatty ethers, polyoxyethylene fatty esters, fatty acids, sulfated fatty acids, phosphated fatty acids, sulfosuccinates, amphoteric surfactants, non-ionic poloxamers, non-ionic meroxapols, petroleum derivatives, aliphatic amines, polysiloxane derivatives, sorbitan fatty acid esters, laureth-4, PEG-2 dilaurate, stearic acid, sodium lauryl sulfate, dioctyl sodium sulfosuccinate, cocoamphopropionate, poloxamer 188, meroxapol 258, triethanolamine, dimethicone, polysorbate 60, sorbitan monostearate, pharmaceutically acceptable salts thereof, and combinations thereof.

In some embodiments, a pharmaceutical composition of the present disclosure comprises a peptide of the present disclosure, and a non-ionic surfactant.

For example, in some embodiments, a pharmaceutical composition comprises a peptide of the present disclosure, and a non-ionic surfactant, wherein the non-ionic surfactant can be, by way of non-limiting example, phospholipids, polyoxyl 20 cetostearyl (cetomacrogol), polyoxyethylene 10 stearyl ether and other ceteareth ethers, alkyl poly(ethylene oxide), poloxamers, polysorbates, sodium dioctyl sulfosuccinate, Brij™-30 (Laureth-4), Brij™-58 (Ceteth-20) and Brij™-78 (Steareth-20), Brij™-721 (Steareth-21), Crillet-1 (Polysorbate 20), Crillet-2 (Polysorbate 40), Crillet-3 (Polysorbate 60), Crillet 45 (Polysorbate 80), Myrj-52 (PEG-40 Stearate), Myrj-53 (PEG-50 Stearate), Pluronic™ F77 (Poloxamer 217), Pluronic™ F87 (Poloxamer 237), Pluronic™ F98 (Poloxamer 288), Pluronic™ L62 (Poloxamer 182), Pluronic™ L64 (Poloxamer 184), Pluronic™ F68 (Poloxamer 188), Pluronic™ L81 (Poloxamer 231), Pluronic™ L92 (Poloxamer 282), Pluronic™ L101 (Poloxamer 331), Pluronic™ P103 (Poloxamer 333), Pluracare™ F 108 NF (Poloxamer 338), and Pluracare™ F 127 NF (Poloxamer 407) and combinations thereof.

In some embodiments, a pharmaceutical composition comprises a peptide of the present disclosure, and a cationic surfactant.

For example, in some embodiments, a pharmaceutical composition comprises a peptide of the present disclosure, and a cationic surfactant, wherein the cationic surfactant can be, by way of non-limiting example, benzalkonium chloride, benzethonium chloride, cetyl trimethylammonium bromide, hexadecyl trimethyl ammonium bromide, other alkyltrimethylammonium salts, cetylpyridinium chloride, polyethoxylated tallow, and combinations thereof.

In some embodiments, a pharmaceutical composition of the present disclosure comprising a surfactant may contain an amount of surfactant ranging from about 0.005 wt % to about 99 wt %.

### Thickeners and the Like

In some embodiments, a pharmaceutical composition comprises a peptide of the present disclosure, and a thickener.

In some embodiments, a pharmaceutical composition comprises a peptide of the present disclosure, and a thickener, wherein the thickener can be one or more of the following: natural polysaccharides, semi-synthetic polymers, synthetic polymers, and combinations thereof. Natural polysaccharides include, by way of non-limiting example, acacia, agar, alginates, carrageenan, guar, arabic, tragacanth gum, pectins, dextran, gellan and xanthan gums.

In some embodiments, a pharmaceutical composition comprises a peptide of the present disclosure, and a thickener, wherein the thickener can be one or more semi-synthetic polymers. Examples of semi-synthetic polymers include, by way of non-limiting example, cellulose esters, modified starches, modified celluloses, carboxymethylcellulose, methyl cellulose, ethyl cellulose, hydroxyethyl cellulose, hydroxypropyl cellulose and hydroxypropyl methylcellulose.

In some embodiments, a pharmaceutical composition comprises a peptide of the present disclosure, and a thickener, wherein the thickener can be one or more synthetic polymers. Examples of synthetic polymers include, by way of non-limiting example, polyoxyalkylenes, polyvinyl alcohol, polyacrylamide, polyacrylates, carboxypolymethylene (carbomer), polyvinylpyrrolidone (povidones), polyvinylacetate, polyethylene glycols and poloxamer.

In some embodiments, a pharmaceutical composition comprises a peptide of the present disclosure, and a thickener, wherein the thickener can be one or more of the following: polyoxyethyleneglycol isostearate, cetyl alcohol, stearyl alcohol, Polyglycol 300 isostearate, propyleneglycol, collagen, gelatin, and fatty acids (e.g., lauric acid, myristic acid, palmitic acid, stearic acid, palmitoleic acid, linoleic acid, linolenic acid, oleic acid and the like).

Examples of additional thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly(acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminum oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof.

In some embodiments, the thickener can be a cellulose derivative, e.g., methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

In some embodiments, a pharmaceutical composition of the present disclosure comprising a thickener may contain an amount of thickener ranging from about 0.005 wt % to about 99 wt %.

### Preservatives

In some embodiments, a pharmaceutical composition comprises a peptide of the present disclosure, and one or more preservatives.

In some embodiments, a pharmaceutical composition comprises a peptide of the present disclosure, and one or more preservatives, wherein the one or more preservatives can include, by way of non-limiting example, parabens, ascorbyl palmitate, benzoic acid, butylated hydroxyanisole, butylated hydroxytoluene, chlorobutanol, ethylenediamine, ethylparaben, methylparaben, butyl paraben, propylparaben, monothioglycerol, phenol, phenylethyl alcohol, propylparaben, sodium benzoate, sodium propionate, sodium formaldehyde sulfoxylate, sodium metabisulfite, sorbic acid, sulfur dioxide, maleic acid, propyl gallate, benzalkonium chloride, benzethonium chloride, benzyl alcohol, chlorhexidine acetate, chlorhexidine gluconate, sorbic acid, potassium sorbitol, chlorbutanol, phenoxyethanol, cetylpyridinium chloride, phenylmercuric nitrate, thiomersal, and combinations thereof.

Examples of additional preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In some embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

In some embodiments, a pharmaceutical composition of the present disclosure can comprise a preservative, wherein the preservative is methylparaben or propylparaben.

In some embodiments, a pharmaceutical composition of the present disclosure comprising a preservative may contain an amount of preservative ranging from about 0.005 wt % to about 99 wt %.

### Buffers and pH Modifiers

In some embodiments, a pharmaceutical composition comprises a peptide of the present disclosure, and a buffer or pH adjusting agent.

In some embodiments, a pharmaceutical composition comprises a peptide of the present disclosure, and a buffer or pH adjusting agent, wherein the buffer or pH adjusting agent can be: phosphoric acid, monobasic sodium or potassium phosphate, triethanolamine (TRIS), BICINE, HEPES, Trizma, glycine, histidine, arginine, lysine, asparagine, aspartic acid, glutamine, glutamic acid, carbonate, bicarbonate, potassium metaphosphate, potassium phosphate, monobasic sodium acetate, acetic acid, acetate, citric acid, sodium citrate anhydrous, sodium citrate dihydrate and combinations thereof.

In some embodiments, an acid or a base is added to adjust the pH. Suitable acids or bases include, by way of non-limiting example, HCL, NaOH and KOH.

Examples of buffers include without limitation: phosphate buffer system (sodium dihydrogen phosphate dehydrate, disodium phosphate dodecahydrate, bibasic sodium phosphate, anhydrous monobasic sodium phosphate), bicarbonate buffer system, and bisulfate buffer system.

In some embodiments, a pharmaceutical composition of the present disclosure comprising a buffer may contain an amount of buffer ranging from about 0.005 wt % to about 99 wt %.

In some embodiments, a pharmaceutical composition of the present disclosure comprises a peptide of the present disclosure, and a sodium phosphate buffer.

In some embodiments, a pharmaceutical composition of the present disclosure comprises a peptide of the present disclosure, and a sodium phosphate buffer, wherein the sodium phosphate buffer has a concentration of about 20 mM, and a pH of about 7.0.

In some embodiments, a pharmaceutical composition of the present disclosure comprises a peptide of the present disclosure, and one or more buffer salts.

In some embodiments, a pharmaceutical composition of the present disclosure comprises a peptide of the present disclosure, and one or more buffer salts, wherein the one or more buffer salts is a sodium phosphate monobasic monohydrate, and a sodium phosphate dibasic heptahydrate.

In some embodiments, a pharmaceutical composition comprise a concentration of sodium phosphate monobasic monohydrate ranging from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% by weight of the total composition.

In some embodiments, a pharmaceutical composition of the present disclosure comprises a concentration of sodium phosphate monobasic monohydrate ranging from about 0.1% to about 99.9%; from about 1% to about 99.9%; from about 2% to about 99.9%; from about 3% to about 99.9%; from about 4% to about 99.9%; from about 5% to about 99.9%; from about 6% to about 99.9%; from about 7% to about 99.9%; from about 8% to about 99.9%; from about 9% to about 99.9%; from about 10% to about 99.9%; from about 11% to about 99.9%; from about 12% to about 99.9%; from about 13% to about 99.9%; from about 14% to about 99.9%; from about 15% to about 99.9%; from about 16% to about 99.9%; from about 17% to about 99.9%; from about 18% to about 99.9%; from about 19% to about 99.9%; from about 20% to about 99.9%; from about 21% to about 99.9%; from about 22% to about 99.9%; from about 23% to about 99.9%; from about 24% to about 99.9%; from about 25% to about 99.9%; from about 26% to about 99.9%; from about 27% to about 99.9%; from about 28% to about 99.9%; from about 29% to about 99.9%; from about 30% to about 99.9%; from about 31% to about 99.9%; from about 32% to about 99.9%; from about 33% to about 99.9%; from about 34% to about 99.9%; from about 35% to about 99.9%; from about 36% to about 99.9%; from about 37% to about 99.9%; from about 38% to about 99.9%; from about 39% to about 99.9%; from about 40% to about 99.9%; from about 41% to about 99.9%; from about 42% to about 99.9%; from about 43% to about 99.9%; from about 44% to about 99.9%; from about 45% to about 99.9%; from about 46% to about 99.9%; from about 47% to about 99.9%; from about 48% to about 99.9%; from about 49% to about 99.9%; from about 50% to about 99.9%; from about 51% to about 99.9%; from about 52% to about 99.9%; from about 53% to about 99.9%; from about 54% to about 99.9%; from about 55% to about 99.9%; from about 56% to about 99.9%; from about 57% to about 99.9%; from about 58% to about 99.9%; from about 59% to about 99.9%; from about 60% to about 99.9%; from about 61% to about 99.9%; from about 62% to about 99.9%; from about 63% to about 99.9%; from about 64% to about 99.9%; from about 65% to about 99.9%; from about 66% to about 99.9%; from about 67% to about 99.9%; from about 68% to about 99.9%; from about 69% to about 99.9%; from about 70% to about 99.9%; from about 71% to about 99.9%; from about 72% to about 99.9%; from about 73% to about 99.9%; from about 74% to about 99.9%; from about 75% to about 99.9%; from about 76% to about 99.9%; from about 77% to about 99.9%; from about 78% to about 99.9%; from about 79% to about 99.9%; from about 80% to about 99.9%; from about 81% to about 99.9%; from about 82% to about 99.9%; from about 83% to about 99.9%; from about 84% to about 99.9%; from about 85% to about 99.9%; from about 86% to about 99.9%; from about 87% to about 99.9%; from about 88% to about 99.9%; from about 89% to about 99.9%; from about 90% to about 99.9%; from about 91% to about 99.9%; from about 92% to about 99.9%; from about 93% to about 99.9%; from about 94% to about 99.9%; from about 95% to about 99.9%; from about 96% to about 99.9%; from about 97% to about 99.9%; from about 98% to about 99.9%; or from about 99% to about 99.9%, wt/wt of the total composition.

In some embodiments, a pharmaceutical composition of the present disclosure comprises a concentration of sodium phosphate monobasic monohydrate ranging from about 0.1% to about 99%; from about 0.1% to about 98%; from about 0.1% to about 97%; from about 0.1% to about 96%; from about 0.1% to about 95%; from about 0.1% to about 94%; from about 0.1% to about 93%; from about 0.1% to about 92%; from about 0.1% to about 91%; from about 0.1% to about 90%; from about 0.1% to about 89%; from about 0.1% to about 88%; from about 0.1% to about 87%; from about 0.1% to about 86%; from about 0.1% to about 85%; from about 0.1% to about 84%; from about 0.1% to about 83%; from about 0.1% to about 82%; from about 0.1% to about 81%; from about 0.1% to about 80%; from about 0.1% to about 79%; from about 0.1% to about 78%; from about 0.1% to about 77%; from about 0.1% to about 76%; from about 0.1% to about 75%; from about 0.1% to about 74%; from about 0.1% to about 73%; from about 0.1% to about 72%; from about 0.1% to about 71%; from about 0.1% to about 70%; from about 0.1% to about 69%; from about 0.1% to about 68%; from about 0.1% to about 67%; from about 0.1% to about 66%; from about 0.1% to about 65%; from about 0.1% to about 64%; from about 0.1% to about 63%; from about 0.1% to about 62%; from about 0.1% to about 61%; from about 0.1% to about 60%; from about 0.1% to about 59%; from about 0.1% to about 58%; from about 0.1% to about 57%; from about 0.1% to about 56%; from about 0.1% to about 55%; from about 0.1% to about 54%; from about 0.1% to about 53%; from about 0.1% to about 52%; from about 0.1% to about 51%; from about 0.1% to about 50%; from about 0.1% to about 49%; from about 0.1% to about 48%; from about 0.1% to about 47%; from about 0.1% to about 46%; from about 0.1% to about 45%; from about 0.1% to about 44%; from about 0.1% to about 43%; from about 0.1% to about 42%; from about 0.1% to about 41%; from about 0.1% to about 40%; from about 0.1% to about 39%; from about 0.1% to about 38%; from about 0.1% to about 37%; from about 0.1% to about 36%; from about 0.1% to about 35%; from about 0.1% to about 34%; from about 0.1% to about 33%; from about 0.1% to about 32%; from about 0.1% to about 31%; from about 0.1% to about 30%; from about 0.1% to about 29%; from about 0.1% to about 28%; from about 0.1% to about 27%; from about 0.1% to about 26%; from about 0.1% to about 25%; from about 0.1% to about 24%; from about 0.1% to about 23%; from about 0.1% to about 22%; from about 0.1% to about 21%; from about 0.1% to about 20%; from about 0.1% to about 19%; from about 0.1% to about 18%; from about 0.1% to about 17%; from about 0.1% to about 16%; from about 0.1% to about 15%; from about 0.1% to about 14%; from about 0.1% to about 13%; from about 0.1% to about 12%; from about 0.1% to about 11%; from about 0.1% to about 10%; from about 0.1% to about 9%; from about 0.1% to about 8%; from about 0.1% to about 7%; from about 0.1% to about 6%; from about 0.1% to about 5%; from about 0.1% to about 4%; from about 0.1% to about 3%; from about 0.1% to about 2%; from about 0.1% to about 1%; or from about 0.1% to about 0.5%, wt/wt of the total composition.

In some embodiments, a pharmaceutical composition of the present disclosure comprises a concentration of sodium phosphate dibasic heptahydrate ranging from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% by weight of the total composition.

In some embodiments, a pharmaceutical composition of the present disclosure comprises a concentration of sodium phosphate dibasic heptahydrate ranging from about 0.1% to about 99.9%; from about 1% to about 99.9%; from about 2% to about 99.9%; from about 3% to about 99.9%; from about 4% to about 99.9%; from about 5% to about 99.9%; from about 6% to about 99.9%; from about 7% to about 99.9%; from about 8% to about 99.9%; from about 9% to about 99.9%; from about 10% to about 99.9%; from about 11% to about 99.9%; from about 12% to about 99.9%; from about 13% to about 99.9%; from about 14% to about 99.9%; from about 15% to about 99.9%; from about 16% to about 99.9%; from about 17% to about 99.9%; from about 18% to about 99.9%; from about 19% to about 99.9%; from about 20% to about 99.9%; from about 21% to about 99.9%; from about 22% to about 99.9%; from about 23% to about 99.9%; from about 24% to about 99.9%; from about 25% to about 99.9%; from about 26% to about 99.9%; from about 27% to about 99.9%; from about 28% to about 99.9%; from about 29% to about 99.9%; from about 30% to about 99.9%; from about 31% to about 99.9%; from about 32% to about 99.9%; from about 33% to about 99.9%; from about 34% to about 99.9%; from about 35% to about 99.9%; from about 36% to about 99.9%; from about 37% to about 99.9%; from about 38% to about 99.9%; from about 39% to about 99.9%; from about 40% to about 99.9%; from about 41% to about 99.9%; from about 42% to about 99.9%; from about 43% to about 99.9%; from about 44% to about 99.9%; from about 45% to about 99.9%; from about 46% to about 99.9%; from about 47% to about 99.9%; from about 48% to about 99.9%; from about 49% to about 99.9%; from about 50% to about 99.9%; from about 51% to about 99.9%; from about 52% to about 99.9%; from about 53% to about 99.9%; from about 54% to about 99.9%; from about 55% to about 99.9%; from about 56% to about 99.9%; from about 57% to about 99.9%; from about 58% to about 99.9%; from about 59% to about 99.9%; from about 60% to about 99.9%; from about 61% to about 99.9%; from about 62% to about 99.9%; from about 63% to about 99.9%; from about 64% to about 99.9%; from about 65% to about 99.9%; from about 66% to about 99.9%; from about 67% to about 99.9%; from about 68% to about 99.9%; from about 69% to about 99.9%; from about 70% to about 99.9%; from about 71% to about 99.9%; from about 72% to about 99.9%; from about 73% to about 99.9%; from about 74% to about 99.9%; from about 75% to about 99.9%; from about 76% to about 99.9%; from about 77% to about 99.9%; from about 78% to about 99.9%; from about 79% to about 99.9%; from about 80% to about 99.9%; from about 81% to about 99.9%; from about 82% to about 99.9%; from about 83% to about 99.9%; from about 84% to about 99.9%; from about 85% to about 99.9%; from about 86% to about 99.9%; from about 87% to about 99.9%; from about 88% to about 99.9%; from about 89% to about 99.9%; from about 90% to about 99.9%; from about 91% to about 99.9%; from about 92% to about 99.9%; from about 93% to about 99.9%; from about 94% to about 99.9%; from about 95% to about 99.9%; from about 96% to about 99.9%; from about 97% to about 99.9%; from about 98% to about 99.9%; or from about 99% to about 99.9%, wt/wt of the total composition.

In some embodiments, a pharmaceutical composition of the present disclosure comprises a concentration of sodium phosphate dibasic heptahydrate ranging from about 0.1% to about 99%; from about 0.1% to about 98%; from about 0.1% to about 97%; from about 0.1% to about 96%; from about 0.1% to about 95%; from about 0.1% to about 94%; from about 0.1% to about 93%; from about 0.1% to about 92%; from about 0.1% to about 91%; from about 0.1% to about 90%; from about 0.1% to about 89%; from about 0.1% to about 88%; from about 0.1% to about 87%; from about 0.1% to about 86%; from about 0.1% to about 85%; from about 0.1% to about 84%; from about 0.1% to about 83%; from about 0.1% to about 82%; from about 0.1% to about 81%; from about 0.1% to about 80%; from about 0.1% to about 79%; from about 0.1% to about 78%; from about 0.1% to about 77%; from about 0.1% to about 76%; from about 0.1% to about 75%; from about 0.1% to about 74%; from about 0.1% to about 73%; from about 0.1% to about 72%; from about 0.1% to about 71%; from about 0.1% to about 70%; from about 0.1% to about 69%; from about 0.1% to about 68%; from about 0.1% to about 67%; from about 0.1% to about 66%; from about 0.1% to about 65%; from about 0.1% to about 64%; from about 0.1% to about 63%; from about 0.1% to about 62%; from about 0.1% to about 61%; from about 0.1% to about 60%; from about 0.1% to about 59%; from about 0.1% to about 58%; from about 0.1% to about 57%; from about 0.1% to about 56%; from about 0.1% to about 55%; from about 0.1% to about 54%; from about 0.1% to about 53%; from about 0.1% to about 52%; from about 0.1% to about 51%; from about 0.1% to about 50%; from about 0.1% to about 49%; from about 0.1% to about 48%; from about 0.1% to about 47%; from about 0.1% to about 46%; from about 0.1% to about 45%; from about 0.1% to about 44%; from about 0.1% to about 43%; from about 0.1% to about 42%; from about 0.1% to about 41%; from about 0.1% to about 40%; from about 0.1% to about 39%; from about 0.1% to about 38%; from about 0.1% to about 37%; from about 0.1% to about 36%; from about 0.1% to about 35%; from about 0.1% to about 34%; from about 0.1% to about 33%; from about 0.1% to about 32%; from about 0.1% to about 31%; from about 0.1% to about 30%; from about 0.1% to about 29%; from about 0.1% to about 28%; from about 0.1% to about 27%; from about 0.1% to about 26%; from about 0.1% to about 25%; from about 0.1% to about 24%; from about 0.1% to about 23%; from about 0.1% to about 22%; from about 0.1% to about 21%; from about 0.1% to about 20%; from about 0.1% to about 19%; from about 0.1% to about 18%; from about 0.1% to about 17%; from about 0.1% to about 16%; from about 0.1% to about 15%; from about 0.1% to about 14%; from about 0.1% to about 13%; from about 0.1% to about 12%; from about 0.1% to about 11%; from about 0.1% to about 10%; from about 0.1% to about 9%; from about 0.1% to about 8%; from about 0.1% to about 7%; from about 0.1% to about 6%; from about 0.1% to about 5%; from about 0.1% to about 4%; from about 0.1% to about 3%; from about 0.1% to about 2%; from about 0.1% to about 1%; or from about 0.1% to about 0.5%, wt/wt of the total composition.

In some embodiments, a pharmaceutical composition of the present disclosure comprises a peptide of the present disclosure, and one or more pH modifiers.

In some embodiments, a pharmaceutical composition of the present disclosure comprises a peptide of the present disclosure, and one or more pH modifiers, wherein the one or more pH modifiers is a sodium hydroxide or a phosphoric acid.

In some embodiments, a pharmaceutical composition of the present disclosure comprises a peptide of the present disclosure, and one or more pH modifiers, wherein the one or more pH modifiers is a sodium hydroxide or a phosphoric acid, and wherein the sodium hydroxide or phosphoric acid have a concentration of about 1N.

In some embodiments, the sodium hydroxide or phosphoric acid are an amount required to adjust the pH of the pharmaceutical composition to about 6.8 to about 7.2.

### Solvents

In some embodiments, a pharmaceutical composition comprises a peptide of the present disclosure, and a solvent.

In some embodiments, the solvent can be selected from: water, Ringer's solution, lactated Ringer's solution and isotonic sodium chloride solution. Other examples of solvents include, without limitation, sterile, fixed oils which are conventionally employed as a solvent or suspending medium, and a variety of bland fixed oils including, for example, synthetic mono- or diglycerides.

In some embodiments, a solvent can be fatty acids such as oleic acid find use in the preparation of injectables.

In some embodiments, the solvent can be selected from: glycerol, ethylene glycol, propylene glycol, polyethylene glycol and polypropylene glycol.

In some embodiments, a pharmaceutical composition comprises a peptide of the present disclosure, and a solvent, wherein the solvent is water.

In some embodiments, a pharmaceutical composition of the present disclosure comprises a concentration of water ranging from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% by weight of the total composition.

In some embodiments, a pharmaceutical composition of the present disclosure comprises a concentration of water ranging from about 0.1% to about 99.9%; from about 1% to about 99.9%; from about 2% to about 99.9%; from about 3% to about 99.9%; from about 4% to about 99.9%; from about 5% to about 99.9%; from about 6% to about 99.9%; from about 7% to about 99.9%; from about 8% to about 99.9%; from about 9% to about 99.9%; from about 10% to about 99.9%; from about 11% to about 99.9%; from about 12% to about 99.9%; from about 13% to about 99.9%; from about 14% to about 99.9%; from about 15% to about 99.9%; from about 16% to about 99.9%; from about 17% to about 99.9%; from about 18% to about 99.9%; from about 19% to about 99.9%; from about 20% to about 99.9%; from about 21% to about 99.9%; from about 22% to about 99.9%; from about 23% to about 99.9%; from about 24% to about 99.9%; from about 25% to about 99.9%; from about 26% to about 99.9%; from about 27% to about 99.9%; from about 28% to about 99.9%; from about 29% to about 99.9%; from about 30% to about 99.9%; from about 31% to about 99.9%; from about 32% to about 99.9%; from about 33% to about 99.9%; from about 34% to about 99.9%; from about 35% to about 99.9%; from about 36% to about 99.9%; from about 37% to about 99.9%; from about 38% to about 99.9%; from about 39% to about 99.9%; from about 40% to about 99.9%; from about 41% to about 99.9%; from about 42% to about 99.9%; from about 43% to about 99.9%; from about 44% to about 99.9%; from about 45% to about 99.9%; from about 46% to about 99.9%; from about 47% to about 99.9%; from about 48% to about 99.9%; from about 49% to about 99.9%; from about 50% to about 99.9%; from about 51% to about 99.9%; from about 52% to about 99.9%; from about 53% to about 99.9%; from about 54% to about 99.9%; from about 55% to about 99.9%; from about 56% to about 99.9%; from about 57% to about 99.9%; from about 58% to about 99.9%; from about 59% to about 99.9%; from about 60% to about 99.9%; from about 61% to about 99.9%; from about 62% to about 99.9%; from about 63% to about 99.9%; from about 64% to about 99.9%; from about 65% to about 99.9%; from about 66% to about 99.9%; from about 67% to about 99.9%; from about 68% to about 99.9%; from about 69% to about 99.9%; from about 70% to about 99.9%; from about 71% to about 99.9%; from about 72% to about 99.9%; from about 73% to about 99.9%; from about 74% to about 99.9%; from about 75% to about 99.9%; from about 76% to about 99.9%; from about 77% to about 99.9%; from about 78% to about 99.9%; from about 79% to about 99.9%; from about 80% to about 99.9%; from about 81% to about 99.9%; from about 82% to about 99.9%; from about 83% to about 99.9%; from about 84% to about 99.9%; from about 85% to about 99.9%; from about 86% to about 99.9%; from about 87% to about 99.9%; from about 88% to about 99.9%; from about 89% to about 99.9%; from about 90% to about 99.9%; from about 91% to about 99.9%; from about 92% to about 99.9%; from about 93% to about 99.9%; from about 94% to about 99.9%; from about 95% to about 99.9%; from about 96% to about 99.9%; from about 97% to about 99.9%; from about 98% to about 99.9%; or from about 99% to about 99.9%, wt/wt of the total composition.

In some embodiments, a pharmaceutical composition of the present disclosure comprises a concentration of water ranging from about 0.1% to about 99%; from about 0.1% to about 98%; from about 0.1% to about 97%; from about 0.1% to about 96%; from about 0.1% to about 95%; from about 0.1% to about 94%; from about 0.1% to about 93%; from about 0.1% to about 92%; from about 0.1% to about 91%; from about 0.1% to about 90%; from about 0.1% to about 89%; from about 0.1% to about 88%; from about 0.1% to about 87%; from about 0.1% to about 86%; from about 0.1% to about 85%; from about 0.1% to about 84%; from about 0.1% to about 83%; from about 0.1% to about 82%; from about 0.1% to about 81%; from about 0.1% to about 80%; from about 0.1% to about 79%; from about 0.1% to about 78%; from about 0.1% to about 77%; from about 0.1% to about 76%; from about 0.1% to about 75%; from about 0.1% to about 74%; from about 0.1% to about 73%; from about 0.1% to about 72%; from about 0.1% to about 71%; from about 0.1% to about 70%; from about 0.1% to about 69%; from about 0.1% to about 68%; from about 0.1% to about 67%; from about 0.1% to about 66%; from about 0.1% to about 65%; from about 0.1% to about 64%; from about 0.1% to about 63%; from about 0.1% to about 62%; from about 0.1% to about 61%; from about 0.1% to about 60%; from about 0.1% to about 59%; from about 0.1% to about 58%; from about 0.1% to about 57%; from about 0.1% to about 56%; from about 0.1% to about 55%; from about 0.1% to about 54%; from about 0.1% to about 53%; from about 0.1% to about 52%; from about 0.1% to about 51%; from about 0.1% to about 50%; from about 0.1% to about 49%; from about 0.1% to about 48%; from about 0.1% to about 47%; from about 0.1% to about 46%; from about 0.1% to about 45%; from about 0.1% to about 44%; from about 0.1% to about 43%; from about 0.1% to about 42%; from about 0.1% to about 41%; from about 0.1% to about 40%; from about 0.1% to about 39%; from about 0.1% to about 38%; from about 0.1% to about 37%; from about 0.1% to about 36%; from about 0.1% to about 35%; from about 0.1% to about 34%; from about 0.1% to about 33%; from about 0.1% to about 32%; from about 0.1% to about 31%; from about 0.1% to about 30%; from about 0.1% to about 29%; from about 0.1% to about 28%; from about 0.1% to about 27%; from about 0.1% to about 26%; from about 0.1% to about 25%; from about 0.1% to about 24%; from about 0.1% to about 23%; from about 0.1% to about 22%; from about 0.1% to about 21%; from about 0.1% to about 20%; from about 0.1% to about 19%; from about 0.1% to about 18%; from about 0.1% to about 17%; from about 0.1% to about 16%; from about 0.1% to about 15%; from about 0.1% to about 14%; from about 0.1% to about 13%; from about 0.1% to about 12%; from about 0.1% to about 11%; from about 0.1% to about 10%; from about 0.1% to about 9%; from about 0.1% to about 8%; from about 0.1% to about 7%; from about 0.1% to about 6%; from about 0.1% to about 5%; from about 0.1% to about 4%; from about 0.1% to about 3%; from about 0.1% to about 2%; from about 0.1% to about 1%; or from about 0.1% to about 0.5%, wt/wt of the total composition.

### Disintegrants

In some embodiments, a pharmaceutical composition comprises a peptide of the present disclosure, and a disintegrant.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

In some embodiments, a pharmaceutical composition of the present disclosure comprising a disintegrant may contain an amount of disintegrant ranging from about 0.005 wt % to about 99 wt %.

### Glidants and Lubricants

In some embodiments, a pharmaceutical composition comprises a peptide of the present disclosure, and a glidant.

In some embodiments, a pharmaceutical composition comprises a peptide of the present disclosure, and a lubricant.

In some embodiments, a pharmaceutical composition comprises a peptide of the present disclosure, and a lubricant, wherein the lubricant can be, e.g., a natural or synthetic fat or oil (e.g., a tris-fatty acid glycerate and the like).

In some embodiments, a pharmaceutical composition comprises a peptide of the present disclosure, and a lubricant, wherein the lubricant can be glycerin (also called glycerine, glycerol, 1,2,3-propanetriol, and trihydroxypropane), polyethylene glycols (PEGs), polypropylene glycol, polyisobutene, polyethylene oxide, behenic acid, behenyl alcohol, sorbitol, mannitol, lactose, polydimethylsiloxane and combinations thereof.

Examples of additional glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

In some embodiments, a pharmaceutical composition of the present disclosure comprising a glidant may contain an amount of glidant ranging from about 0.005 wt % to about 99 wt %.

### Diluents, Fillers, and Bulking Agents

In some embodiments, a pharmaceutical composition comprises a peptide of the present disclosure, and a diluent.

In some embodiments, a pharmaceutical composition comprises a peptide of the present disclosure, and a filler.

In some embodiments, a pharmaceutical composition comprises a peptide of the present disclosure, and a bulking agent.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

In some embodiments, a pharmaceutical composition of the present disclosure comprising a diluent may contain an amount of diluent ranging from about 0.005 wt % to about 99 wt %.

### Binders

In some embodiments, a pharmaceutical composition comprises a peptide of the present disclosure, and a binder.

Examples of binders include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, a pharmaceutical composition of the present disclosure comprising a binder may contain an amount of binder ranging from about 0.005 wt % to about 99 wt %.

### Emollients

In some embodiments, a pharmaceutical composition of the present disclosure can comprise an emollient.

In some embodiments, a pharmaceutical composition of the present disclosure can comprise an emollient such as lanolin alcohol, lanolin, lanolin derivatives, cholesterol, petrolatum, isostearyl neopentanoate and mineral oils.

In some embodiments, a pharmaceutical composition of the present disclosure can comprise an emollient such as mineral oil, mixtures of mineral oil and lanolin alcohols, cetyl alcohol, stearyl alcohol, cetostearyl alcohol, petrolatum, petrolatum and lanolin alcohols, cetyl esters wax, cholesterol, glycerin, glyceryl monostearate, isopropyl myristate, isopropyl palmitate, lecithin, allyl caproate, althea officinalis extract, arachidyl alcohol, argobase EUC, Butylene glycol dicaprylate/dicaprate, acacia, allantoin, carrageenan, cetyl dimethicone, cyclomethicone, diethyl succinate, dihydroabietyl behenate, dioctyl adipate, ethyl laurate, ethyl palmitate, ethyl stearate, isoamyl laurate, octanoate, PEG-75 lanolin, sorbitan laurate, walnut oil, wheat germ oil super refined almond, super refined sesame, super refined soybean, octyl palmitate, caprylic/capric triglyceride and glyceryl cocoate.

Examples of emollients are well known in the art. Additional examples of emollients include, without limitation, triglyceride esters, fatty acid esters and amides, waxes such as beeswax, spermaceti, or carnauba wax, phospholipids such as lecithin, and sterols and fatty acid esters thereof.

In some embodiments, a pharmaceutical composition of the present disclosure can comprise an emollient, wherein the emollient is white petrolatum, or white wax.

In some embodiments, a pharmaceutical composition of the present disclosure comprising an emollient may contain an amount of emollient, ranging from about 0.005 wt % to about 99 wt %.

### Stiffening Agents

In some embodiments, a pharmaceutical composition of the present disclosure can comprise a stiffening agent, e.g., an agent capable of stiffening a formulation of the invention, for example, by increasing the viscosity of the formulation.

In some embodiments, a pharmaceutical composition of the present disclosure can comprise a stiffening agent, e.g., stearyl alcohol, cetostearyl alcohol, polyoxylene (10) stearyl ether, mono- or diglycerides, and/or cetyl alcohol.

In some embodiments, a pharmaceutical composition of the present disclosure comprising an stiffening agent may contain an amount of stiffening agent, ranging from about 0.005 wt % to about 99 wt %.

### Chelating Agents

In some embodiments, a pharmaceutical composition of the present disclosure can comprise a chelating agent.

Exemplary chelating agents include, without limitation, ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

In some embodiments, a pharmaceutical composition of the present disclosure can comprise a chelating agent, wherein the chelating agent is disodium EDTA.

In some embodiments, a pharmaceutical composition of the present disclosure comprising a chelating agent may contain an amount of chelating agent ranging from about 0.005 wt % to about 99 wt %.

### Emulsifiers

In some embodiments, a pharmaceutical composition of the present disclosure can comprise an emulsifier.

In some embodiments, a pharmaceutical composition of the present disclosure can comprise an emulsifier such as nonionic, anionic, cationic, amphoteric, polymeric, synthetic emulsifiers, and/or mixtures thereof.

In some embodiments, the emulsifier can comprise a polysorbate, an alkyl sulfate, Lipowax® D, or combinations thereof. Suitable polysorbate compounds include, polysorbate 20, 40, 60, 80, or combinations thereof, such as Tween® 20, 40, 60, 80, or combinations thereof.

In some embodiments, the emulsifier can comprise natural emulsifiers, such as acacia, gelatin, lecithin and cholesterol; finely dispersed solids, such as colloidal clays, bentonite, veegum (magnesium aluminum silicate; and synthetic emulsifiers, such as salts of fatty acids, sulfates such as sorbitan trioleate, sorbitan tristearate, sucrose distearate, propylene glycol monostearate, glycerol monostearate, propylene glycol monolaurate, sorbitan monostearate, sorbitan monolaurate, polyoxyethylene-4-lauryl ether, sodium lauryl sulfate, sulfonates such as dioctyl sodium sulfosuccinate, glyceryl esters, polyoxyethylene glycol esters and ethers, diethylene glycol monostearate, PEG 200 distearate, and sorbitan fatty acid esters, such as sorbitan monopalmitate, and their polyoxyethylene derivatives, polyoxyethylene glycol esters such as the monostearate, Polysorbate 80 (ethoxylated sorbitan monooleate) (supplied by Spectrum, etc.); and combinations thereof.

In some embodiments, a pharmaceutical composition of the present disclosure can comprise stearyl alcohol.

In some embodiments, a pharmaceutical composition of the present disclosure can comprise emulsifying wax.

In some embodiments, a pharmaceutical composition of the present disclosure comprising an emulsifier may contain an amount of emulsifier ranging from about 0.005 wt % to about 99 wt %.

### Formulations and Routes of Administration

In some embodiments, the peptides described herein, or a pharmaceutical composition thereof, can be formulated into a variety of forms for delivery to subject in need thereof by any accepted route of administration.

### Rectal Administration: Generally

In some embodiments, the peptides of the present disclosure, or pharmaceutical compositions thereof, are suitable for local administration, e.g., local administration by way of topically administering the peptide of the present disclosure, or a pharmaceutical composition thereof, at a particular treatment site, (e.g., the digestive tract, the gastrointestinal (“GI”) tract) so as to provide local administration of the chemical entity to the area in need of treatment (e.g., GI tract). Examples of such compositions include, without limitation, compositions for rectal administration.

In some embodiments, the peptides of the present disclosure, or pharmaceutical compositions thereof, are suitable for local administration to the GI tract.

In some embodiments, the peptides of the present disclosure, or pharmaceutical compositions thereof, are suitable for local administration to one or more specific locations within the digestive or GI tract. For example, in some embodiments, at least some of the peptides of the present disclosure, or pharmaceutical compositions thereof, is present in the lower GI tract (e.g., the large intestine, e.g., the colon, e.g., the ascending colon and/or transverse colon and/or distal colon; or the small bowel).

In some embodiments, at least some of the peptides of the present disclosure, or pharmaceutical compositions thereof, is present in the ascending colon and/or the transverse colon and/or the distal colon. Methods of said local administration can include, without limitation, rectal administration.

In certain embodiments, the peptides of the present disclosure, or pharmaceutical compositions thereof, are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmaceutically acceptable excipients usable in a pharmaceutical composition of the present disclosure formulated for rectal administration, e.g., such as a gel, cream, enema, rectal foam, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

### Suppositories

In certain embodiments, a pharmaceutical composition of the present disclosure can be formulated as suppositories,

In some embodiments, suppositories can be prepared by mixing the peptides of the present disclosure, or pharmaceutical compositions thereof, with suitable non-irritating excipients or carriers.

For example, in some embodiments, suppositories can be prepared by mixing the peptides of the present disclosure, or pharmaceutical compositions thereof, with suitable non-irritating excipients or carriers such as cocoa butter, polyethylene glycol or a suppository wax which are solid at ambient temperature but liquid at body temperature and therefore melt in the rectum and release the active compound.

In some embodiments, a suppository formulation may contain an amount of a peptide of the present disclosure, or a pharmaceutically acceptable salt thereof, ranging from about 0.001 wt % to about 5.00 wt %.

### Enema Formulations and Enema Kits

In some embodiments, a peptide of the present disclosure, or a pharmaceutical composition thereof, can be formulated as an enema.

In some embodiments, an enema formulation may contain an amount of a peptide of the present disclosure, or a pharmaceutically acceptable salt thereof, ranging from about 0.001 wt % to about 5.00 wt %.

In some embodiments, enema formulations containing the peptides of the present disclosure, or pharmaceutical compositions thereof, can be provided in “ready-to-use” form.

In some embodiments, enema formulations containing the peptides of the present disclosure, or pharmaceutical compositions thereof, are provided in one or more kits or packs.

In certain embodiments, the kit or pack includes two or more separately contained/packaged components, e.g. two components, which, when mixed together, provide the desired formulation (e.g., as a suspension).

In some embodiments, the present disclosure provides a two component system that includes a first component and a second component, wherein: (i) the first component (e.g., contained in a sachet) includes the peptide of the present disclosure (as described anywhere herein), and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, water, glycerol, ethylene glycol, propylene glycol, polyethylene glycol and polypropylene glycol. In other embodiments, each of the one or more liquids is water. In other embodiments, each of the one or more liquids is an oil, e.g. natural and/or synthetic oils that are commonly used in pharmaceutical preparations.

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In some embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In some embodiments, each of the one or more pharmaceutically acceptable excipients can be independently selected from thickeners, viscosity enhancing agents, bulking agents, mucoadhesive agents, buffers, preservatives, and fillers.

In some embodiments, each of the one or more pharmaceutically acceptable excipients can be independently selected from diluents, binders, lubricants, glidants, and disintegrants.

Exemplary thickeners, viscosity enhancing agents, and mucoadhesive agents, for enema formulations, include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly(acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminum oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. In some embodiments, examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Exemplary preservatives for enema formulations, include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In some embodiments, the preservative for an enema formulation is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Exemplary buffers for enema formulations, include without limitation: phosphate buffer system (sodium dihydrogen phosphate dehydrate, disodium phosphate dodecahydrate, bibasic sodium phosphate, anhydrous monobasic sodium phosphate), bicarbonate buffer system, and bisulfate buffer system.

Exemplary disintegrants for enema formulations include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp).

Exemplary glidants and lubricants (aggregation inhibitors) for enema formulations include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Exemplary diluents, also referred to as “fillers” or “bulking agents” for enema formulations, include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Exemplary binders for enema formulations, include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

### Rectal Gels

In some embodiments, the pharmaceutical compositions described herein are formulated as rectal gels.

In some embodiments, the rectal gels are suitable for the regional or local non-systemic administration of one or more of the peptides of the present disclosure to the rectum and/or colon.

In some embodiments, rectal gel formulations comprise a peptide of the present disclosure, dissolved or suspended in a solvent/liquid carrier vehicle.

In some embodiments, rectal gel formulations comprise a peptide of the present disclosure, dissolved or suspended in a solvent/liquid carrier vehicle, and at least one thickening agents.

In certain embodiments a rectal gel formulations can further comprise one or more of the following: a buffering agent(s), a preservative(s), and an antioxidant(s).

In certain embodiments, rectal gels have gel-like consistencies but are sufficiently flowable so as to be capable of local or regional administration through a catheter, needle, syringe, or other comparable means of local or regional administration.

In some embodiments, rectal gel formulation may contain an amount of a peptide of the present disclosure, or a pharmaceutically acceptable salt thereof, ranging from about 0.001 wt % to about 5.00 wt %.

### Foams

In some embodiments, a pharmaceutical composition of the present disclosure can be formulated as a foam or a mousse (e.g., a pharmaceutical rectal foam composition).

As used herein, “foam” refers to a coarse dispersion of gas in liquid in which the volume of the gas is considerably larger than that of the liquid. Accordingly, a foam is a tightly packed aggregation of gas bubbles, separated from each other by thin films of liquid (lamellae). The existence and stability of a foam depends on a surface layer of solute molecules. At the surface of a liquid, molecules are in a state of dynamic equilibrium, in which the net attractive forces exerted by the bulk of the fluid cause molecules to move out of the surface; this motion is counterbalanced by ordinary diffusion back into the diluted surface layer. The equilibrium results in the surface layer being constantly less dense than the bulk fluid, which creates a state of tension at the surface. The tension can be somewhat relieved by adsorption of foreign molecules either out of the bulk solution, or out of the vapor phase.

In some embodiments, a pharmaceutical composition of the present disclosure (e.g., a pharmaceutical rectal foam composition), can be formulated as a foam, wherein the foam may or may not be propellant-based (i.e., substantially propellant-free or propellant-free).

In some embodiments, a pharmaceutical composition of the present disclosure (e.g., a pharmaceutical rectal foam composition), can further comprise one or more propellants as described herein.

In some embodiments, the addition of propellants to a pharmaceutical composition of the present disclosure results in a foamable formulations via manual aeration. In some embodiments, the addition of one or more propellants to a pharmaceutical composition of the present disclosure can provide a more consistent delivery of the active agent. For example, addition of a propellant to a foamable formulation may be useful in producing metered dosing of the composition.

Various properties of the pharmaceutical compositions formulated as a foam (e.g., a pharmaceutical rectal foam composition) described herein can be assessed by methods known in the art. For example, one or more properties of foam expansion, foam cling, foam inversion, foam density, and foam collapse, and can be assessed by methods known in the art.

In some embodiments, a pharmaceutical composition formulated as a foam (e.g., a pharmaceutical rectal foam composition) will expand gradually and expand to a large volume such that it is uniformly distributed internally over the intended area of treatment.

In some embodiments, a pharmaceutical composition formulated as a foam (e.g., a pharmaceutical rectal foam composition) exhibits good retention (“foam cling”).

In some embodiments, a pharmaceutical composition formulated as a foam (e.g., a pharmaceutical rectal foam composition) exhibits superior foam inversion properties. Foam inversion is another measure of foam retention properties, e.g., cohesiveness and/or adhesiveness of the foam formulations.

In some embodiments, a pharmaceutical composition formulated as a foam (e.g., a pharmaceutical rectal foam composition) exhibits good foam density. Foam density can be measured as the weight of foam per unit volume.

In some embodiments, a pharmaceutical composition formulated as a foam (e.g., a pharmaceutical rectal foam composition) exhibits good foam collapse properties. In some embodiments, foam collapse is a measure of how quickly and for how long the active components of the foam formulation will come into contact with a locus to be treated or locus of administration (e.g. mucosa).

Exemplary descriptions of foams and the properties thereof are described in U.S. Pat. Nos. 10,092,588, and 11,103,454; the disclosures of which are incorporated herein by reference in their entireties.

In some embodiments, the pharmaceutical compositions are formulated as rectal foams (e.g., a pharmaceutical rectal foam composition).

In some embodiments, rectal foams are used for the rectal administration and for local or non-systemic delivery of the peptides of the present disclosure to the rectum and/or colon.

In some embodiments, a pharmaceutical rectal foam composition comprises a peptide of the present disclosure, dissolved or suspended in a liquid carrier vehicle.

In some embodiments, a pharmaceutical rectal foam composition comprises a peptide of the present disclosure, dissolved or suspended in a liquid carrier vehicle, and a surfactant/emulsifier with foaming properties.

In some embodiments, a pharmaceutical rectal foam composition comprises a peptide of the present disclosure, dissolved or suspended in a liquid carrier vehicle, and a surfactant/emulsifier with foaming properties, and a propellant (e.g., a propellant gas).

In certain embodiments, a pharmaceutical rectal foam composition can comprise one or more of the following: a suspending/solubilizing agent, a thickener, a preservative, a chelating agent, a buffer, an antioxidant, a tonicity modifiers, and/or a spreading agent.

In some embodiments, surfactants/emulsifiers include, by way of non-limiting example, non-ionic surfactants, anionic surfactants, cationic surfactants, and combinations thereof.

In some embodiments, a rectal foam formulation may contain an amount of a peptide of the present disclosure, or a pharmaceutically acceptable salt thereof, ranging from about 0.001 wt % to about 5.00 wt %.

In some embodiments, a pharmaceutical composition of the present disclosure can comprise Methylparaben.

In some embodiments, a pharmaceutical composition of the present disclosure can comprise Propylparaben

In some embodiments, a pharmaceutical composition of the present disclosure can comprise Propylparaben in an amount that is about 0.02% w/w of the total composition.

In some embodiments, a pharmaceutical composition of the present disclosure can comprise Methylparaben in an amount that is about 0.18% w/w of the total composition.

In some embodiments, a pharmaceutical composition of the present disclosure can comprise Propylene Glycol in an amount that is about 0.164% w/w of the total composition.

In some embodiments, a pharmaceutical composition of the present disclosure can comprise Purified Water in an amount that is about 77.5495% w/w of the total composition.

In some embodiments, a pharmaceutical composition of the present disclosure can comprise Propylene Glycol in an amount that is about 10.0000% w/w of the total composition.

In some embodiments, a pharmaceutical composition of the present disclosure can comprise Sodium Phosphate Dibasic in an amount that is about 0.1640% w/w of the total composition.

In some embodiments, a pharmaceutical composition of the present disclosure can comprise Sodium Dihydrogen Phosphate Monohydrate in an amount that is about 0.1165% w/w of the total composition.

In some embodiments, a pharmaceutical composition of the present disclosure can comprise Disodium EDTA in an amount that is about 0.0500% w/w of the total composition.

In some embodiments, a pharmaceutical composition of the present disclosure can comprise Methylparaben in an amount that is about 0.1000% w/w of the total composition.

In some embodiments, a pharmaceutical composition of the present disclosure can comprise Light Mineral Oil in an amount that is about 6.0000% w/w of the total composition.

In some embodiments, a pharmaceutical composition of the present disclosure can comprise Isopropyl Myristate in an amount that is about 0.5000% w/w of the total composition.

In some embodiments, a pharmaceutical composition of the present disclosure can comprise White Petrolatum in an amount that is about 1.0000% w/w of the total composition.

In some embodiments, a pharmaceutical composition of the present disclosure can comprise Polyoxyl 20 Cetostearyl Ether in an amount that is about 2.5000% w/w of the total composition.

In some embodiments, a pharmaceutical composition of the present disclosure can comprise Cetyl Alcohol in an amount that is about 1.0000% w/w of the total composition.

In some embodiments, a pharmaceutical composition of the present disclosure can comprise Stearyl Alcohol in an amount that is about 1.0000% w/w of the total composition.

In some embodiments, a pharmaceutical composition of the present disclosure can comprise Propylparaben in an amount that is about 0.0200% w/w of the total composition.

In some embodiments, a pharmaceutical composition of the present disclosure can comprise emulsifying wax in an amount that is about 1.4% w/w of the total composition.

In some embodiments, a pharmaceutical composition of the present disclosure can comprise Polyoxylene (10) Stearyl Ether in an amount that is about 1.4% w/w of the total composition.

In some embodiments, a pharmaceutical composition of the present disclosure can comprise Purified Water in an amount that is about 77.5495% w/w of the total composition.

In some embodiments, a pharmaceutical composition of the present disclosure can comprise Propylene Glycol in an amount that is about 10.0000% w/w of the total composition.

In some embodiments, a pharmaceutical composition of the present disclosure can comprise Sodium Phosphate Dibasic in an amount that is about 0.1640% w/w of the total composition.

In some embodiments, a pharmaceutical composition of the present disclosure can comprise Sodium Dihydrogen Phosphate Monohydrate in an amount that is about 0.1165% w/w of the total composition.

In some embodiments, a pharmaceutical composition of the present disclosure can comprise Disodium EDTA in an amount that is about 0.0500% w/w of the total composition.

In some embodiments, a pharmaceutical composition of the present disclosure can comprise Methylparaben in an amount that is about 0.1000% w/w of the total composition.

In some embodiments, a pharmaceutical composition of the present disclosure can comprise Light Mineral Oil in an amount that is about 7.0000% w/w of the total composition.

In some embodiments, a pharmaceutical composition of the present disclosure can comprise White Wax in an amount that is about 0.5000% w/w of the total composition.

In some embodiments, a pharmaceutical composition of the present disclosure can comprise Mono and/or Di-Glycerides in an amount that is about 2.5000% w/w of the total composition.

In some embodiments, a pharmaceutical composition of the present disclosure can comprise Cetyl Alcohol in an amount that is about 1.0000% w/w of the total composition.

In some embodiments, a pharmaceutical composition of the present disclosure can comprise Stearyl Alcohol in an amount that is about 1.0000% w/w of the total composition.

In some embodiments, a pharmaceutical composition of the present disclosure can comprise Propylparaben in an amount that is about 0.0200% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of purified water ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of propylene glycol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of sodium phosphate dibasic ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of sodium dihydrogen phosphate monohydrate ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of disodium EDTA ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of methylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of light mineral oil ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of isopropyl myristate ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of white petrolatum ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of polyoxyl 20 cetostearyl ether ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of cetyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of stearyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of propylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of purified water ranging from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; or from about 90% to about 10% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of purified water ranging from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; or from about 80% to about 20% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of purified water ranging from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; or from about 90% to about 10% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of purified water ranging from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; or from about 80% to about 20% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of propylene glycol ranging from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of propylene glycol ranging from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of propylene glycol ranging from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of propylene glycol ranging from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of sodium phosphate dibasic ranging from about 0.08% to about 99.92%; from about 0.09% to about 99.91%; from about 0.10% to about 99.90%; from about 0.15% to about 99.85%; from about 0.20% to about 99.80%; or from about 0.25% to about 99.75%, w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of sodium phosphate dibasic ranging from about 0.10% to about 99.90%; from about 0.15% to about 99.85%; or from about 0.18% to about 99.82% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of sodium phosphate dibasic ranging from about 0.08% to about 99.92%; from about 0.09% to about 99.91%; from about 0.10% to about 99.90%; from about 0.15% to about 99.85%; from about 0.20% to about 99.80%; or from about 0.25% to about 99.75%, w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of sodium phosphate dibasic ranging from about 0.10% to about 99.90%; from about 0.15% to about 99.85%; or from about 0.18% to about 99.82% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of sodium dihydrogen phosphate monohydrate ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of sodium dihydrogen phosphate monohydrate ranging from about 0.07% to about 99.93%; from about 0.08% to about 99.92%; from about 0.09% to about 99.91%; from about 0.10% to about 99.90%; or from about 0.15% to about 99.85% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of sodium dihydrogen phosphate monohydrate ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of sodium dihydrogen phosphate monohydrate ranging from about 0.07% to about 99.93%; from about 0.08% to about 99.92%; from about 0.09% to about 99.91%; from about 0.10% to about 99.90%; or from about 0.15% to about 99.85% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of disodium EDTA ranging from about 0.02% to about 99.98%; from about 0.03% to about 99.97%; from about 0.04% to about 99.96%; from about 0.05% to about 99.95%; from about 0.06% to about 99.94%; from about 0.07% to about 99.93%; from about 0.08% to about 99.92%; from about 0.09% to about 99.91%; or from about 0.10% to about 99.90% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of disodium EDTA ranging from about 0.04% to about 99.96%; from about 0.05% to about 99.95%; or from about 0.06% to about 99.94% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of disodium EDTA ranging from about 0.02% to about 99.98%; from about 0.03% to about 99.97%; from about 0.04% to about 99.96%; from about 0.05% to about 99.95%; from about 0.06% to about 99.94%; from about 0.07% to about 99.93%; from about 0.08% to about 99.92%; from about 0.09% to about 99.91%; or from about 0.10% to about 99.90% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of disodium EDTA ranging from about 0.04% to about 99.96%; from about 0.05% to about 99.95%; or from about 0.06% to about 99.94% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of methylparaben ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of methylparaben ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85%; w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of methylparaben ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of methylparaben ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85%; w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of light mineral oil ranging from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; or from about 12% to about 88% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of light mineral oil ranging from about 4% to about 96%; from about 5% to about 95%; from about 6% to about 94%; from about 7% to about 93%; or from about 8% to about 92%; w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of light mineral oil ranging from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; or from about 12% to about 88% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of light mineral oil ranging from about 4% to about 96%; from about 5% to about 95%; from about 6% to about 94%; from about 7% to about 93%; or from about 8% to about 92%; w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of isopropyl myristate ranging from about 0.02% to about 99.98%; from about 0.03% to about 99.97%; from about 0.04% to about 99.96%; from about 0.05% to about 99.95%; from about 0.06% to about 99.94%; from about 0.07% to about 99.93%; from about 0.08% to about 99.92%; from about 0.09% to about 99.91%; or from about 0.10% to about 99.90% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of isopropyl myristate ranging from about 0.04% to about 99.96%; from about 0.05% to about 99.95%; from about 0.06% to about 99.94%; from about 0.07% to about 99.93%; from about 0.08% to about 99.92% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of isopropyl myristate ranging from about 0.02% to about 99.98%; from about 0.03% to about 99.97%; from about 0.04% to about 99.96%; from about 0.05% to about 99.95%; from about 0.06% to about 99.94%; from about 0.07% to about 99.93%; from about 0.08% to about 99.92%; from about 0.09% to about 99.91%; or from about 0.10% to about 99.90% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of isopropyl myristate ranging from about 0.04% to about 99.96%; from about 0.05% to about 99.95%; from about 0.06% to about 99.94%; from about 0.07% to about 99.93%; from about 0.08% to about 99.92% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of white petrolatum ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of white petrolatum ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of white petrolatum ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of white petrolatum ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of polyoxyl 20 cetostearyl ether ranging from about 1% to about 99%; from about 2% to about 98%; from about 3% to about 97%; from about 4% to about 96%; or from about 5% to about 95% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of polyoxyl 20 cetostearyl ether ranging from about 1% to about 99%; from about 1.5% to about 99.5%; from about 2% to about 98%; from about 2.5 to about 97.5%; or from about 3% to about 97% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of polyoxyl 20 cetostearyl ether ranging from about 1% to about 99%; from about 2% to about 98%; from about 3% to about 97%; from about 4% to about 96%; or from about 5% to about 95% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of polyoxyl 20 cetostearyl ether ranging from about 1% to about 99%; from about 1.5% to about 99.5%; from about 2% to about 98%; from about 2.5 to about 97.5%; or from about 3% to about 97% w/w of the total composition.

In some preferred embodiments, a pharmaceutical composition of the present disclosure can comprise a peptide of the present disclosure, polyoxyl 20 cetostearyl ether, cetyl alcohol, stearyl alcohol; and optionally further comprising purified water, propylene glycol, sodium phosphate dibasic, sodium dihydrogen phosphate monohydrate, disodium EDTA, methylparaben, light mineral oil, isopropyl myristate, white petrolatum, and/or propylparaben; said composition having an amount of polyoxyl 20 cetostearyl ether ranging from about 1% to about 99%; from about 2% to about 98%; from about 3% to about 97%; from about 4% to about 96%; or from about 5% to about 95% w/w of the total composition.

In some preferred embodiments, a pharmaceutical composition of the present disclosure can comprise a peptide of the present disclosure, polyoxyl 20 cetostearyl ether, cetyl alcohol, stearyl alcohol; and optionally further comprising purified water, propylene glycol, sodium phosphate dibasic, sodium dihydrogen phosphate monohydrate, disodium EDTA, methylparaben, light mineral oil, isopropyl myristate, white petrolatum, and/or propylparaben; said composition having an amount of polyoxyl 20 cetostearyl ether ranging from about 1% to about 99%; from about 1.5% to about 99.5%; from about 2% to about 98%; from about 2.5 to about 97.5%; or from about 3% to about 97% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of cetyl alcohol ranging from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of cetyl alcohol ranging from about 0.08% to about 99.92%; from about 0.09% to about 99.91%; from about 1% to about 99%; from about 1.5% to about 98.5%; w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of cetyl alcohol ranging from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of cetyl alcohol ranging from about 0.08% to about 99.92%; from about 0.09% to about 99.91%; from about 1% to about 99%; from about 1.5% to about 98.5%; w/w of the total composition.

In some preferred embodiments, a pharmaceutical composition of the present disclosure can comprise a peptide of the present disclosure, polyoxyl 20 cetostearyl ether, cetyl alcohol, stearyl alcohol; and optionally further comprising purified water, propylene glycol, sodium phosphate dibasic, sodium dihydrogen phosphate monohydrate, disodium EDTA, methylparaben, light mineral oil, isopropyl myristate, white petrolatum, and/or propylparaben; said composition having an amount of cetyl alcohol ranging from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98% w/w of the total composition.

In some preferred embodiments, a pharmaceutical composition of the present disclosure can comprise a peptide of the present disclosure, polyoxyl 20 cetostearyl ether, cetyl alcohol, stearyl alcohol; and optionally further comprising purified water, propylene glycol, sodium phosphate dibasic, sodium dihydrogen phosphate monohydrate, disodium EDTA, methylparaben, light mineral oil, isopropyl myristate, white petrolatum, and/or propylparaben; said composition having an amount of cetyl alcohol ranging from about 0.08% to about 99.92%; from about 0.09% to about 99.91%; from about 1% to about 99%; from about 1.5% to about 98.5%; w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of stearyl alcohol ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of stearyl alcohol ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of stearyl alcohol ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an amount of stearyl alcohol ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some preferred embodiments, a pharmaceutical composition of the present disclosure can comprise a peptide of the present disclosure, polyoxyl 20 cetostearyl ether, cetyl alcohol, stearyl alcohol; and optionally further comprising purified water, propylene glycol, sodium phosphate dibasic, sodium dihydrogen phosphate monohydrate, disodium EDTA, methylparaben, light mineral oil, isopropyl myristate, white petrolatum, and/or propylparaben; said composition having an amount of stearyl alcohol ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some preferred embodiments, a pharmaceutical composition of the present disclosure can comprise a peptide of the present disclosure, polyoxyl 20 cetostearyl ether, cetyl alcohol, stearyl alcohol; and optionally further comprising purified water, propylene glycol, sodium phosphate dibasic, sodium dihydrogen phosphate monohydrate, disodium EDTA, methylparaben, light mineral oil, isopropyl myristate, white petrolatum, and/or propylparaben; said composition having an amount of stearyl alcohol ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of propylparaben ranging from about 0.01% to about 99.99%; from about 0.02% to about 99.98%; from about 0.03% to about 99.97%; from about 0.04% to about 99.96%; from about 0.05% to about 99.95% w/w of the total composition.

In some preferred embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, isopropyl myristate, white petrolatum, polyoxyl 20 cetostearyl ether, cetyl alcohol, stearyl alcohol, and propylparaben; having an amount of propylparaben ranging from about 0.015% to about 99.99%; from about 0.02% to about 99.98%; from about 0.03% to about 99.97%; w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of propylparaben ranging from about 0.01% to about 99.99%; from about 0.02% to about 99.98%; from about 0.03% to about 99.97%; from about 0.04% to about 99.96%; from about 0.05% to about 99.95% w/w of the total composition.

In some preferred embodiments, a pharmaceutical composition of the present disclosure can comprise a peptide of the present disclosure, purified water, propylene glycol, light mineral oil, isopropyl myristate, white petrolatum, polyoxyl 20 cetostearyl ether, cetyl alcohol, stearyl alcohol; and optionally further comprising sodium phosphate dibasic; sodium dihydrogen phosphate monohydrate; disodium EDTA; methylparaben; and/or propylparaben; said composition having an optional amount of propylparaben ranging from about 0.015% to about 99.99%; from about 0.02% to about 99.98%; from about 0.03% to about 99.97%; w/w of the total composition.

In some embodiments, a pharmaceutical composition of the present disclosure can comprise a peptide of the present disclosure in an amount ranging from about 50 μg to about 3000 μg; and purified water in an amount ranging from about 50% to about 90%, propylene glycol in an amount ranging from about 5% to about 20%, sodium phosphate dibasic in an amount ranging from about 0.08% to about 0.25%, sodium dihydrogen phosphate monohydrate in an amount ranging from about 0.05% to about 0.20%, disodium EDTA in an amount ranging from about 0.02% to about 0.10%, methylparaben in an amount ranging from about 0.05% to about 0.2%, light mineral oil in an amount ranging from about 3% to about 12%, isopropyl myristate in an amount ranging from about 0.2% to about 1%, white petrolatum in an amount ranging from about 0.5% to about 2%, polyoxyl 20 cetostearyl ether in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.05%, w/w % of the total composition.

In some embodiments, a pharmaceutical composition of the present disclosure can comprise a peptide of the present disclosure in an amount of about 50 μg, about 100 μg, about 300 μg, about 600 μg, about 900 μg, about 1800 μg, about 2500 μg, or about 3000 μg; and purified water in an amount ranging from about 50% to about 90%, propylene glycol in an amount ranging from about 5% to about 20%, sodium phosphate dibasic in an amount ranging from about 0.08% to about 0.25%, sodium dihydrogen phosphate monohydrate in an amount ranging from about 0.05% to about 0.20%, disodium EDTA in an amount ranging from about 0.02% to about 0.10%, methylparaben in an amount ranging from about 0.05% to about 0.2%, light mineral oil in an amount ranging from about 3% to about 12%, isopropyl myristate in an amount ranging from about 0.2% to about 1%, white petrolatum in an amount ranging from about 0.5% to about 2%, polyoxyl 20 cetostearyl ether in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.05%, w/w % of the total composition.

In some embodiments, a pharmaceutical composition of the present disclosure can comprise a peptide of the present disclosure in an amount ranging from about 100 μg to about 2500 μg; and purified water in an amount ranging from about 60% to about 80%, propylene glycol in an amount ranging from about 7% to about 15%, sodium phosphate dibasic in an amount ranging from about 0.1% to about 0.18%, sodium dihydrogen phosphate monohydrate in an amount ranging from about 0.07% to about 0.15%, disodium EDTA in an amount ranging from about 0.04% to about 0.6%, methylparaben in an amount ranging from about 0.08% to about 0.15%, light mineral oil in an amount ranging from about 4% to about 8%, isopropyl myristate in an amount ranging from about 0.4% to about 0.8%, white petrolatum in an amount ranging from about 0.8% to about 1.5%, polyoxyl 20 cetostearyl ether in an amount ranging from about 1.5% to about 3%, cetyl alcohol in an amount ranging from about 0.8% to about 1.5%, stearyl alcohol in an amount ranging from about 0.8% to about 1.5%, and propylparaben in an amount ranging from about 0.015% to about 0.03%, w/w % of the total composition.

In some embodiments, a pharmaceutical composition of the present disclosure can comprise a peptide of the present disclosure in an amount of about 100 μg, about 300 μg, about 600 μg, about 900 μg, about 1800 μg, or about 2500 μg; and purified water in an amount ranging from about 60% to about 80%, propylene glycol in an amount ranging from about 7% to about 15%, sodium phosphate dibasic in an amount ranging from about 0.1% to about 0.18%, sodium dihydrogen phosphate monohydrate in an amount ranging from about 0.07% to about 0.15%, disodium EDTA in an amount ranging from about 0.04% to about 0.6%, methylparaben in an amount ranging from about 0.08% to about 0.15%, light mineral oil in an amount ranging from about 4% to about 8%, isopropyl myristate in an amount ranging from about 0.4% to about 0.8%, white petrolatum in an amount ranging from about 0.8% to about 1.5%, polyoxyl 20 cetostearyl ether in an amount ranging from about 1.5% to about 3%, cetyl alcohol in an amount ranging from about 0.8% to about 1.5%, stearyl alcohol in an amount ranging from about 0.8% to about 1.5%, and propylparaben in an amount ranging from about 0.015% to about 0.03%, w/w % of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of purified water ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of propylene glycol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of sodium phosphate dibasic ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of sodium dihydrogen phosphate monohydrate ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of disodium EDTA ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of methylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of emulsifying wax ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of polyoxylene (10) stearyl ether ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of cetyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of propylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of purified water ranging from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; or from about 90% to about 10% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of purified water ranging from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; or from about 85% to about 15% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of purified water ranging from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; or from about 90% to about 10% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of purified water ranging from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; or from about 85% to about 15% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of propylene glycol ranging from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of propylene glycol ranging from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; or from about 20% to about 80% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of propylene glycol ranging from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of propylene glycol ranging from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; or from about 20% to about 80% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of sodium phosphate dibasic ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; from about 0.20% to about 99.80%; from about 0.21% to about 99.79%; from about 0.22% to about 99.78%; from about 0.23% to about 99.77%; from about 0.24% to about 99.76%; from about 0.25% to about 99.75%; from about 0.26% to about 99.74%; from about 0.27% to about 99.73%; from about 0.28% to about 99.72%; from about 0.29% to about 99.71%; or from about 0.30% to about 99.70% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of sodium phosphate dibasic ranging from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of sodium phosphate dibasic ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; from about 0.20% to about 99.80%; from about 0.21% to about 99.79%; from about 0.22% to about 99.78%; from about 0.23% to about 99.77%; from about 0.24% to about 99.76%; from about 0.25% to about 99.75%; from about 0.26% to about 99.74%; from about 0.27% to about 99.73%; from about 0.28% to about 99.72%; from about 0.29% to about 99.71%; or from about 0.30% to about 99.70% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of sodium phosphate dibasic ranging from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of sodium dihydrogen phosphate monohydrate ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of sodium dihydrogen phosphate monohydrate ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of sodium dihydrogen phosphate monohydrate ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of sodium dihydrogen phosphate monohydrate ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of disodium EDTA ranging from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; or from about 1% to about 99% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of disodium EDTA ranging from about 0.04% to about 99.96%; from about 0.05% to about 99.95; from about 0.06% to about 99.94%; from about 0.07% to about 99.93%; from about 0.08% to about 99.92% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of disodium EDTA ranging from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; or from about 1% to about 99% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of disodium EDTA ranging from about 0.04% to about 99.96%; from about 0.05% to about 99.95; from about 0.06% to about 99.94%; from about 0.07% to about 99.93%; from about 0.08% to about 99.92% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of methylparaben ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of methylparaben ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of methylparaben ranging from about 0.05% to about 99.95%; from about 0.06% to about 99.94%; from about 0.07% to about 99.93%; from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; from about 0.15% to about 99.85%; from about 0.16% to about 99.84%; from about 0.17% to about 99.83%; from about 0.18% to about 99.82%; from about 0.19% to about 99.81%; or from about 0.20% to about 99.80% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of methylparaben ranging from about 0.08% to about 99.92%; from about 0.09% to about 99.91%; from about 0.10% to about 99.90%; from about 0.11% to about 99.89%; from about 0.12% to about 99.88%; from about 0.13% to about 99.87%; from about 0.14% to about 99.86%; or from about 0.15% to about 99.85% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of emulsifying wax ranging from about 0.7% to about 99.93%; from about 0.8% to about 99.92%; from about 0.9% to about 99.91%; from about 1% to about 99%; from about 2% to about 98%; or from about 3% to about 97% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of emulsifying wax ranging from about 1% to about 99%; or from about 2% to about 98% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of emulsifying wax ranging from about 0.7% to about 99.93%; from about 0.8% to about 99.92%; from about 0.9% to about 99.91%; from about 1% to about 99%; from about 2% to about 98%; or from about 3% to about 97% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of emulsifying wax ranging from about 1% to about 99%; or from about 2% to about 98% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of polyoxylene (10) stearyl ether ranging from about 0.7% to about 99.93%; from about 0.8% to about 99.92%; from about 0.9% to about 99.91%; from about 1% to about 99%; from about 2% to about 98%; or from about 3% to about 97% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of polyoxylene (10) stearyl ether ranging from about 1% to about 99%; or from about 2% to about 98% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of polyoxylene (10) stearyl ether ranging from about 0.7% to about 99.93%; from about 0.8% to about 99.92%; from about 0.9% to about 99.91%; from about 1% to about 99%; from about 2% to about 98%; or from about 3% to about 97% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of polyoxylene (10) stearyl ether ranging from about 1% to about 99%; or from about 2% to about 98% w/w of the total composition.

In some embodiments, a pharmaceutical composition of the present disclosure can comprise a peptide of the present disclosure, polyoxylene (10) stearyl ether, cetyl alcohol; and optionally further comprise purified water, propylene glycol, sodium phosphate dibasic, sodium dihydrogen phosphate monohydrate, disodium EDTA, methylparaben, emulsifying wax, and/or propylparaben; said composition having an amount of polyoxylene (10) stearyl ether ranging from about 0.7% to about 99.93%; from about 0.8% to about 99.92%; from about 0.9% to about 99.91%; from about 1% to about 99%; from about 2% to about 98%; or from about 3% to about 97% w/w of the total composition.

In some embodiments, a pharmaceutical composition of the present disclosure can comprise a peptide of the present disclosure, polyoxylene (10) stearyl ether, cetyl alcohol; and optionally further comprise purified water, propylene glycol, sodium phosphate dibasic, sodium dihydrogen phosphate monohydrate, disodium EDTA, methylparaben, emulsifying wax, and/or propylparaben; said composition having an amount of polyoxylene (10) stearyl ether ranging from about 1% to about 99%; or from about 2% to about 98% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of cetyl alcohol ranging from about 0.4% to about 99.6%; from about 0.5% to about 99.5%; from about 0.6% to about 99.4%; from about 0.7% to about 99.3%; from about 0.8% to about 99.2%; from about 0.9% to about 99.1%; from about 0.10% to about 99.90%; from about 1.1% to about 98.9%; from about 1.2% to about 98.8%; from about 1.3% to about 98.7%; or from about 1.4% to about 98.6% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of cetyl alcohol ranging from about 0.6% to about 99.4%; from about 0.7% to about 99.3%; from about 0.8% to about 99.2%; or from about 0.9% to about 99.1% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of cetyl alcohol ranging from about 0.4% to about 99.6%; from about 0.5% to about 99.5%; from about 0.6% to about 99.4%; from about 0.7% to about 99.3%; from about 0.8% to about 99.2%; from about 0.9% to about 99.1%; from about 0.10% to about 99.90%; from about 1.1% to about 98.9%; from about 1.2% to about 98.8%; from about 1.3% to about 98.7%; or from about 1.4% to about 98.6% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an amount of cetyl alcohol ranging from about 0.6% to about 99.4%; from about 0.7% to about 99.3%; from about 0.8% to about 99.2%; or from about 0.9% to about 99.1% w/w of the total composition.

In some embodiments, a pharmaceutical composition of the present disclosure can comprise a peptide of the present disclosure, polyoxylene (10) stearyl ether, cetyl alcohol; and optionally further comprise purified water, propylene glycol, sodium phosphate dibasic, sodium dihydrogen phosphate monohydrate, disodium EDTA, methylparaben, emulsifying wax, and/or propylparaben; said composition having an amount of cetyl alcohol ranging from about 0.4% to about 99.6%; from about 0.5% to about 99.5%; from about 0.6% to about 99.4%; from about 0.7% to about 99.3%; from about 0.8% to about 99.2%; from about 0.9% to about 99.1%; from about 0.10% to about 99.90%; from about 1.1% to about 98.9%; from about 1.2% to about 98.8%; from about 1.3% to about 98.7%; or from about 1.4% to about 98.6% w/w of the total composition having an amount of cetyl alcohol ranging from about 0.6% to about 99.4%; from about 0.7% to about 99.3%; from about 0.8% to about 99.2%; or from about 0.9% to about 99.1% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of propylparaben ranging from about 0.01% to about 99.99%; from about 0.02% to about 99.98%; from about 0.03% to about 99.97%; or from about 0.04% to about 99.96% w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, emulsifying wax, polyoxylene (10) stearyl ether, cetyl alcohol, and propylparaben; having an amount of propylparaben ranging from about 0.015% to about 99.995%; from about 0.02% to about 99.980%; from about 0.025% to about 99.975%; or from about 0.03% to about 99.97% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of propylparaben ranging from about 0.01% to about 99.99%; from about 0.02% to about 99.98%; from about 0.03% to about 99.97%; or from about 0.04% to about 99.96% w/w of the total composition.

In some embodiments, a pharmaceutical composition of the present disclosure can comprise a peptide of the present disclosure, purified water, propylene glycol, emulsifying wax, polyoxylene (10) stearyl ether, cetyl alcohol; and optionally comprising sodium phosphate dibasic, sodium dihydrogen phosphate monohydrate, disodium EDTA, methylparaben and/or propylparaben; having an optional amount of propylparaben ranging from about 0.015% to about 99.995%; from about 0.02% to about 99.980%; from about 0.025% to about 99.975%; or from about 0.03% to about 99.97% w/w of the total composition.

In some embodiments, a pharmaceutical composition of the present disclosure can comprise a peptide of the present disclosure in an amount ranging from about 50 μg to about 3000 μg; and purified water in an amount ranging from about 50% to about 90%, propylene glycol in an amount ranging from about 8% to about 30%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, emulsifying wax in an amount ranging from about 0.7% to about 3%, polyoxylene (10) stearyl ether in an amount ranging from about 0.7% to about 3%, cetyl alcohol in an amount ranging from about 0.4% to about 1.4%, and propylparaben in an amount ranging from about 0.01% to about 0.04%, w/w % of the total composition.

In some embodiments, a pharmaceutical composition of the present disclosure can comprise a peptide of the present disclosure in an amount of about 50 μg, about 100 μg, about 300 μg, about 600 μg, about 900 μg, about 1800 μg, about 2500 μg, or about 3000 μg; and purified water in an amount ranging from about 50% to about 90%, propylene glycol in an amount ranging from about 8% to about 30%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, emulsifying wax in an amount ranging from about 0.7% to about 3%, polyoxylene (10) stearyl ether in an amount ranging from about 0.7% to about 3%, cetyl alcohol in an amount ranging from about 0.4% to about 1.4%, and propylparaben in an amount ranging from about 0.01% to about 0.04%, w/w % of the total composition.

In some embodiments, a pharmaceutical composition of the present disclosure can comprise a peptide of the present disclosure in an amount ranging from about 100 μg to about 2500 μg; and purified water in an amount ranging from about 60% to about 85%, propylene glycol in an amount ranging from about 10% to about 20%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, emulsifying wax in an amount ranging from about 1% to about 2%, polyoxylene (10) stearyl ether in an amount ranging from about 1% to about 2%, cetyl alcohol in an amount ranging from about 0.6% to about 0.9%, and propylparaben in an amount ranging from about 0.015% to about 0.03%, w/w % of the total composition.

In some embodiments, a pharmaceutical composition of the present disclosure can comprise a peptide of the present disclosure in an amount of about 100 μg, about 300 μg, about 600 μg, about 900 μg, about 1800 μg, or about 2500 μg; and purified water in an amount ranging from about 60% to about 85%, propylene glycol in an amount ranging from about 10% to about 20%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, emulsifying wax in an amount ranging from about 1% to about 2%, polyoxylene (10) stearyl ether in an amount ranging from about 1% to about 2%, cetyl alcohol in an amount ranging from about 0.6% to about 0.9%, and propylparaben in an amount ranging from about 0.015% to about 0.03%, w/w % of the total composition.

In some embodiments, a pharmaceutical composition of the present disclosure can comprise a peptide of the present disclosure in an amount ranging from about 50 μg to about 3000 μg; and polyoxylene (10) stearyl ether in an amount ranging from about 0.7% to about 3%, cetyl alcohol in an amount ranging from about 0.4% to about 1.4% w/w % of the total composition.

In some embodiments, a pharmaceutical composition of the present disclosure can comprise a peptide of the present disclosure in an amount ranging from about 50 μg to about 3000 μg; polyoxylene (10) stearyl ether in an amount ranging from about 0.7% to about 3%, cetyl alcohol in an amount ranging from about 0.4% to about 1.4% w/w % of the total composition; and optionally further comprise purified water in an amount ranging from about 50% to about 90%, propylene glycol in an amount ranging from about 8% to about 30%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, emulsifying wax in an amount ranging from about 0.7% to about 3%, and propylparaben in an amount ranging from about 0.01% to about 0.04%, w/w % of the total composition.

In some embodiments, a pharmaceutical composition of the present disclosure can comprise a peptide of the present disclosure in an amount of about 50 μg, about 100 μg, about 300 μg, about 600 μg, about 900 μg, about 1800 μg, about 2500 μg, or about 3000 μg; and polyoxylene (10) stearyl ether in an amount ranging from about 0.7% to about 3%, cetyl alcohol in an amount ranging from about 0.4% to about 1.4%, w/w % of the total composition.

In some embodiments, a pharmaceutical composition of the present disclosure can comprise a peptide of the present disclosure in an amount of about 50 μg, about 100 μg, about 300 μg, about 600 μg, about 900 μg, about 1800 μg, about 2500 μg, or about 3000 μg; and polyoxylene (10) stearyl ether in an amount ranging from about 0.7% to about 3%, cetyl alcohol in an amount ranging from about 0.4% to about 1.4%, w/w % of the total composition; and optionally further comprise purified water in an amount ranging from about 50% to about 90%, propylene glycol in an amount ranging from about 8% to about 30%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, emulsifying wax in an amount ranging from about 0.7% to about 3%, and propylparaben in an amount ranging from about 0.01% to about 0.04%, w/w % of the total composition.

In some embodiments, a pharmaceutical composition of the present disclosure can comprise a peptide of the present disclosure in an amount ranging from about 100 μg to about 2500 μg; and polyoxylene (10) stearyl ether in an amount ranging from about 1% to about 2%, cetyl alcohol in an amount ranging from about 0.6% to about 0.9%, w/w % of the total composition.

In some embodiments, a pharmaceutical composition of the present disclosure can comprise a peptide of the present disclosure in an amount ranging from about 100 μg to about 2500 μg; and polyoxylene (10) stearyl ether in an amount ranging from about 1% to about 2%, cetyl alcohol in an amount ranging from about 0.6% to about 0.9%, w/w % of the total composition; and optionally further comprise purified water in an amount ranging from about 60% to about 85%, propylene glycol in an amount ranging from about 10% to about 20%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, emulsifying wax in an amount ranging from about 1% to about 2%, and propylparaben in an amount ranging from about 0.015% to about 0.03%, w/w % of the total composition.

In some embodiments, a pharmaceutical composition of the present disclosure can comprise a peptide of the present disclosure in an amount of about 100 μg, about 300 μg, about 600 μg, about 900 μg, about 1800 μg, or about 2500 μg; and polyoxylene (10) stearyl ether in an amount ranging from about 1% to about 2%, cetyl alcohol in an amount ranging from about 0.6% to about 0.9%, w/w % of the total composition.

In some embodiments, a pharmaceutical composition of the present disclosure can comprise a peptide of the present disclosure in an amount of about 100 μg, about 300 μg, about 600 μg, about 900 μg, about 1800 μg, or about 2500 μg; and polyoxylene (10) stearyl ether in an amount ranging from about 1% to about 2%, cetyl alcohol in an amount ranging from about 0.6% to about 0.9%, w/w % of the total composition; and optionally further comprise purified water in an amount ranging from about 60% to about 85%, propylene glycol in an amount ranging from about 10% to about 20%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, emulsifying wax in an amount ranging from about 1% to about 2%, and propylparaben in an amount ranging from about 0.015% to about 0.03%, w/w % of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of purified water ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of propylene glycol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of sodium phosphate dibasic ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of sodium dihydrogen phosphate monohydrate ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of disodium EDTA ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of methylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of light mineral oil ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of white wax ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of mono- and di-glycerides ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of cetyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of stearyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure, purified water, propylene glycol, sodium phosphate dibasic, sodium dihydrogen phosphate monohydrate, disodium EDTA, methylparaben, light mineral oil, white wax, mono- and di-glycerides, cetyl alcohol, stearyl alcohol, and propylparaben; having an amount of propylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical composition of the present disclosure can comprise a peptide of the present disclosure in an amount ranging from about 50 μg to about 3000 μg; and purified water in an amount ranging from about 50% to about 90%, propylene glycol in an amount ranging from about 5% to about 20%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, light mineral oil in an amount ranging from about 3% to about 14%, white wax in an amount ranging from about 0.2% to about 1%, mono- and di-glycerides in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.04%, w/w % of the total composition.

In some embodiments, a pharmaceutical composition of the present disclosure can comprise a peptide of the present disclosure in an amount of about 50 μg, about 100 μg, about 300 μg, about 600 μg, about 900 μg, about 1800 μg, about 2500 μg, or about 3000 μg; and purified water in an amount ranging from about 50% to about 90%, propylene glycol in an amount ranging from about 5% to about 20%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, light mineral oil in an amount ranging from about 3% to about 14%, white wax in an amount ranging from about 0.2% to about 1%, mono- and di-glycerides in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.04%, w/w % of the total composition.

In some embodiments, a pharmaceutical composition of the present disclosure can comprise a peptide of the present disclosure in an amount ranging from about 100 μg to about 2500 μg; and purified water in an amount ranging from about 60% to about 80%, propylene glycol in an amount ranging from about 8% to about 15%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, light mineral oil in an amount ranging from about 5% to about 9%, white wax in an amount ranging from about 0.4% to about 0.8%, mono- and di-glycerides in an amount ranging from about 2% to about 3%, cetyl alcohol in an amount ranging from about 0.8% to about 1.5%, stearyl alcohol in an amount ranging from about 0.8% to about 1.5%, and propylparaben in an amount ranging from about 0.015% to about 0.03%, w/w % of the total composition.

In some embodiments, a pharmaceutical composition of the present disclosure can comprise a peptide of the present disclosure in an amount of about 100 μg, about 300 μg, about 600 μg, about 900 μg, about 1800 μg, or about 2500 μg; and purified water in an amount ranging from about 60% to about 80%, propylene glycol in an amount ranging from about 8% to about 15%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, light mineral oil in an amount ranging from about 5% to about 9%, white wax in an amount ranging from about 0.4% to about 0.8%, mono- and di-glycerides in an amount ranging from about 2% to about 3%, cetyl alcohol in an amount ranging from about 0.8% to about 1.5%, stearyl alcohol in an amount ranging from about 0.8% to about 1.5%, and propylparaben in an amount ranging from about 0.015% to about 0.03%, w/w % of the total composition.

### Foam Propellants

In some embodiments, pharmaceutical rectal foam compositions are filled in pressurized containers (e.g., a pressurized gas container such as an aerosol canister) prior to rectal administration. In some embodiments the pressurized gas container is a canister. In certain embodiments, propellants used herein include, by way of non-limiting example, hydrocarbons (such as isobutane, N-butane or propane), fluorocarbons (e.g. dichlorodifluoromethane and dichlorotetrafluoroethane), chlorofluorocarbons, dimethyl ether, hydrofluorocarbons, compressed gases, freon (such as freon 12, freon 114), hydrochlorofluorocarbons, hydrofluorocarbons or mixtures thereof.

In some embodiments, the maximum amount of propellant used is determined by its miscibility with other components in the composition to form a mixture, such as a homogeneous mixture. In certain embodiments, the minimal level of propellant used in the composition is determined by the desired foam characteristics, and its ability to substantially or completely evacuate the container.

In some embodiments, the propellant concentration used in a pharmaceutical rectal foam composition is about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 50%, 55% to about 60% (% w/w).

In some embodiments, rectal foams are formed upon rectal administration, wherein the dispensing valve of the can allows rapid expansion of the propellant, triggering the foaming action of the surfactant and resulting foam forms within the rectum and colon. In other embodiments, the rectal foams used for rectal administration of the compositions described herein are formed within the dispensing container prior to rectal administration. The distance the foam can reach within the colon and rectum is controlled by controlling the foam propelling properties by varying the type and quantity of propellant used.

Propellants and gas combinations are shown in the Table A below.

TABLE A
Propellant gas combinations.

| Propellant | PSI | Composition(s)/ratios (%) |
|---|---|---|
| DME | 49-55 | Dimethyl Ether/n-Butane (53/47) |
| A17 | 15-19 | n-Butane (>97) |
| A31 | 29-33 | Isobutane (>95) |
| AP35 | 33-37 | Isobutane (83-89)/n-Butane(5-9)/Propane(5-9) |
| A46 | 44-48 | Isobutane (76.6-88)/Propane (12-21.9) |
| A48 | 46-50 | n-Butane (30-60)/Isobutane (15-30)/ |
| | | Propane (20-45) |
| A70 | 68-72 | Isobutane (46-64)/Propane (35.7-52.3)/ |
| | | n-Butane (0-3) |
| AP70 | 68-72 | Propane (49.8-59.8)/n-Butane (22.3-32.3)/ |
| | | Isobutane(12.9-22.9) |

In some embodiments, a pharmaceutical rectal foam composition of the present disclosure can comprise dimethyl ether (DME).

In some embodiments, a pharmaceutical rectal foam composition of the present disclosure can comprise dimethyl ether (DME) at a PSI of about 49 to about 55.

In some embodiments, a pharmaceutical rectal foam composition of the present disclosure can comprise a propellant comprising Dimethyl Ether and n-Butane.

In some embodiments, a pharmaceutical rectal foam composition of the present disclosure can comprise a propellant comprising a ratio of Dimethyl Ether:n-Butane of 53:47.

In some embodiments, a pharmaceutical rectal foam composition of the present disclosure can comprise A17.

In some embodiments, a pharmaceutical rectal foam composition of the present disclosure can comprise A17 at a PSI of about 15 to about 19.

In some embodiments, a pharmaceutical rectal foam composition of the present disclosure can comprise a propellant comprising n-Butane.

In some embodiments, a pharmaceutical rectal foam composition of the present disclosure can comprise a propellant comprising n-Butane in amount of 97% or more.

In some embodiments, a pharmaceutical rectal foam composition of the present disclosure can comprise A31.

In some embodiments, a pharmaceutical rectal foam composition of the present disclosure can comprise A31 at a PSI of about 29 to about 33.

In some embodiments, a pharmaceutical rectal foam composition of the present disclosure can comprise a propellant comprising isobutane.

In some embodiments, a pharmaceutical rectal foam composition of the present disclosure can comprise a propellant comprising isobutane in amount that is about 95% or greater.

In some embodiments, a pharmaceutical rectal foam composition of the present disclosure can comprise AP35.

In some embodiments, a pharmaceutical rectal foam composition of the present disclosure can comprise AP35 at a PSI of about 33 to about 37.

In some embodiments, a pharmaceutical rectal foam composition of the present disclosure can comprise a propellant comprising Isobutane, n-Butane, and Propane.

In some embodiments, a pharmaceutical rectal foam composition of the present disclosure can comprise a propellant comprising Isobutane (in an amount that is about 83-89%); n-Butane (in an amount that is about 5-9%); and propane (in an amount that is about 5-9%).

In some embodiments, a pharmaceutical rectal foam composition of the present disclosure can comprise A46.

In some embodiments, a pharmaceutical rectal foam composition of the present disclosure can comprise A46 at a PSI of about 44 to about 48.

In some embodiments, a pharmaceutical rectal foam composition of the present disclosure can comprise a propellant comprising Isobutane and Propane.

In some embodiments, a pharmaceutical rectal foam composition of the present disclosure can comprise a propellant comprising Isobutane (in an amount that is about 76.6-88%); and Propane (in an amount that is about 12-21.9%).

In some embodiments, a pharmaceutical rectal foam composition of the present disclosure can comprise A48.

In some embodiments, a pharmaceutical rectal foam composition of the present disclosure can comprise A48 at a PSI of about 46 to about 48.

In some embodiments, a pharmaceutical rectal foam composition of the present disclosure can comprise a propellant comprising Butane; Isobutane; and Propane.

In some embodiments, a pharmaceutical rectal foam composition of the present disclosure can comprise a propellant comprising Butane (in an amount that is about 30-60%); Isobutane (in an amount that is about 15-30%); and Propane (in an amount that is about 20-45%).

In some embodiments, a pharmaceutical rectal foam composition of the present disclosure can comprise A70.

In some embodiments, a pharmaceutical rectal foam composition of the present disclosure can comprise A70 at a PSI of about 68 to about 70.

In some embodiments, a pharmaceutical rectal foam composition of the present disclosure can comprise a propellant comprising Isobutane; Propane; and n-Butane.

In some embodiments, a pharmaceutical rectal foam composition of the present disclosure can comprise a propellant comprising Isobutane (in an amount that is about 46-64%); Propane (in an amount that is about 35.7-52.3%); and n-Butane (in an amount that is about 0-3%).

In some embodiments, a pharmaceutical rectal foam composition of the present disclosure can comprise AP70.

In some embodiments, a pharmaceutical rectal foam composition of the present disclosure can comprise AP70 at a PSI of about 68 to about 72.

In some embodiments, a pharmaceutical rectal foam composition of the present disclosure can comprise a propellant comprising Propane; n-Butane; and Isobutane.

In some embodiments, a pharmaceutical rectal foam composition of the present disclosure can comprise a propellant comprising Propane (in an amount that is about 49.8-59.8%); n-Butane (in an amount that is about 22.3-32.3%); and Isobutane (in an amount that is about 12.9-22.9%).

### Illustrative Pharmaceutical Rectal Foam Compositions

In some embodiments, the pharmaceutical compositions of the present disclosure can be formulated as rectal foams (e.g., a pharmaceutical rectal foam composition of the present disclosure).

In some embodiments, a pharmaceutical rectal foam composition can be used for rectal administration and/or for local or non-systemic delivery of the peptides of the present disclosure to the rectum and/or colon.

In some embodiments, a pharmaceutical rectal foam composition can be used for rectal administration and/or for local or non-systemic delivery of the peptides of the present disclosure to treat a visceral pain condition, e.g., interstitial cystitis/bladder pain syndrome (IC/BPS) or endometriosis.

In some embodiments, a pharmaceutical rectal foam composition can be used for rectal administration and/or for local or non-systemic delivery of the peptides of the present disclosure to reduce the severity of and/or limit the number of symptoms associated with a visceral pain condition, e.g., interstitial cystitis/bladder pain syndrome (IC/BPS) or endometriosis.

In some embodiments, a pharmaceutical rectal foam composition can be used for rectal administration and/or for local or non-systemic delivery of the peptides of the present disclosure to reduce the pain and/or severity of pain associated with a visceral pain condition, e.g., interstitial cystitis/bladder pain syndrome (IC/BPS) or endometriosis.

In some embodiments, a pharmaceutical rectal foam composition comprises a peptide of the present disclosure, dissolved or suspended in a liquid carrier vehicle.

In some embodiments, a pharmaceutical rectal foam composition comprises a peptide of the present disclosure, dissolved or suspended in a liquid carrier vehicle, and a surfactant/emulsifier with foaming properties.

In some embodiments, a pharmaceutical rectal foam composition comprises a peptide of the present disclosure, dissolved or suspended in a liquid carrier vehicle, and a surfactant/emulsifier with foaming properties, and a propellant (e.g., a propellant gas).

In certain embodiments, a pharmaceutical rectal foam composition can comprise one or more of the following: a suspending/solubilizing agent, a thickener, a preservative, a chelating agent, a buffer, an antioxidant, a tonicity modifiers, and/or a spreading agent.

In some embodiments, surfactants/emulsifiers include, by way of non-limiting example, non-ionic surfactants, anionic surfactants, cationic surfactants, and combinations thereof.

In some embodiments, a pharmaceutical rectal foam composition comprises, consists essentially of, or consists of: a peptide or a pharmaceutically acceptable salt, hydrate or solvate thereof, and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

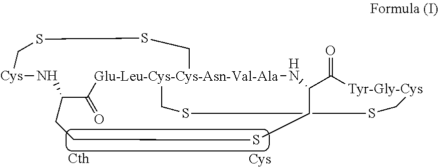

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide is in amount ranging from about 0.001 wt % to about 5.00 wt % of the total composition.

In some embodiments, a pharmaceutical rectal foam composition comprises, consists essentially of, or consists of: a peptide, or a pharmaceutically acceptable salt, hydrate or solvate thereof, and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and wherein the peptide is in amount ranging from about 0.001 wt % to about 5.00 wt % of the total composition.

In some embodiments, a pharmaceutical rectal foam composition comprises, consists essentially of, or consists of: a peptide, or a pharmaceutically acceptable salt, hydrate or solvate thereof, and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and wherein the peptide is in an amount ranging from about 0.002 wt % to about 0.300 wt % of the total composition.

In some embodiments, a pharmaceutical rectal foam composition comprises, consists essentially of, or consists of: a peptide, or a pharmaceutically acceptable salt, hydrate or solvate thereof, and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and wherein the peptide is in an amount ranging from about 0.002 wt % to about 0.300 wt % of the total composition.

In some embodiments, a pharmaceutical rectal foam composition comprises, consists essentially of, or consists of: a peptide, or a pharmaceutically acceptable salt, hydrate or solvate thereof, and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and wherein the peptide is in an amount ranging from about 0.0026 wt % to about 0.2603 wt % of the total composition.

In some embodiments, a pharmaceutical rectal foam composition comprises, consists essentially of, or consists of: a peptide, or a pharmaceutically acceptable salt, hydrate or solvate thereof, and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁o Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and wherein the peptide is in an amount ranging from about 0.0050 wt % to about 0.0500 wt % of the total composition.

In some embodiments, a pharmaceutical rectal foam composition comprises, consists essentially of, or consists of: a peptide, or a pharmaceutically acceptable salt, hydrate or solvate thereof, and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and wherein the peptide is in an amount that is about 0.005%, about 0.006%, about 0.007%, about 0.008%, about 0.0081%, about 0.0082%, about 0.0083%, about 0.0084%, about 0.0085%, about 0.0086%, about 0.0087%, about 0.0088%, about 0.0089%, about 0.009%, about 0.0091%, about 0.0092%, about 0.0093%, about 0.0094%, about 0.0095%, about 0.0096%, about 0.0097%, about 0.0098%, about 0.0099%, about 0.01%, about 0.0101%, about 0.0102%, about 0.0103%, about 0.0104%, about 0.0105%, about 0.0106%, about 0.0107%, about 0.0108%, about 0.0109%, about 0.011%, about 0.0111%, about 0.0112%, about 0.0113%, about 0.0114%, about 0.0115%, about 0.0116%, about 0.0117%, about 0.0118%, about 0.0119%, about 0.012%, about 0.0121%, about 0.0122%, about 0.0123%, about 0.0124%, about 0.0125%, about 0.0126%, about 0.0127%, about 0.0128%, about 0.0129%, about 0.013%, about 0.0131%, about 0.0132%, about 0.0133%, about 0.0134%, about 0.0135%, about 0.0136%, about 0.0137%, about 0.0138%, about 0.0139%, about 0.014%, about 0.0141%, about 0.0142%, about 0.0143%, about 0.0144%, about 0.0145%, about 0.0146%, about 0.0147%, about 0.0148%, about 0.0149%, about 0.015%, about 0.0151%, about 0.0152%, about 0.0153%, about 0.0154%, about 0.0155%, about 0.0156%, about 0.0157%, about 0.0158%, about 0.0159%, about 0.016%, about 0.0161%, about 0.0162%, about 0.0163%, about 0.0164%, about 0.0165%, about 0.0166%, about 0.0167%, about 0.0168%, about 0.0169%, about 0.017%, about 0.0171%, about 0.0172%, about 0.0173%, about 0.0174%, about 0.0175%, about 0.0176%, about 0.0177%, about 0.0178%, about 0.0179%, about 0.018%, about 0.0181%, about 0.0182%, about 0.0183%, about 0.0184%, about 0.0185%, about 0.0186%, about 0.0187%, about 0.0188%, about 0.0189%, about 0.019%, about 0.0191%, about 0.0192%, about 0.0193%, about 0.0194%, about 0.0195%, about 0.0196%, about 0.0197%, about 0.0198%, about 0.0199%, about 0.02%, about 0.0201%, about 0.0202%, about 0.0203%, about 0.0204%, about 0.0205%, about 0.0206%, about 0.0207%, about 0.0208%, about 0.0209%, about 0.021%, about 0.0211%, about 0.0212%, about 0.0213%, about 0.0214%, about 0.0215%, about 0.0216%, about 0.0217%, about 0.0218%, about 0.0219%, about 0.022%, about 0.0221%, about 0.0222%, about 0.0223%, about 0.0224%, about 0.0225%, about 0.0226%, about 0.0227%, about 0.0228%, about 0.0229%, about 0.023%, about 0.0231%, about 0.0232%, about 0.0233%, about 0.0234%, about 0.0235%, about 0.0236%, about 0.0237%, about 0.0238%, about 0.0239%, about 0.024%, about 0.0241%, about 0.0242%, about 0.0243%, about 0.0244%, about 0.0245%, about 0.0246%, about 0.0247%, about 0.0248%, about 0.0249%, about 0.025%, about 0.0251%, about 0.0252%, about 0.0253%, about 0.0254%, about 0.0255%, about 0.0256%, about 0.0257%, about 0.0258%, about 0.0259%, about 0.026%, about 0.0261%, about 0.0262%, about 0.0263%, about 0.0264%, about 0.0265%, about 0.0266%, about 0.0267%, about 0.0268%, about 0.0269%, about 0.027%, about 0.0271%, about 0.0272%, about 0.0273%, about 0.0274%, about 0.0275%, about 0.0276%, about 0.0277%, about 0.0278%, about 0.0279%, about 0.028%, about 0.0281%, about 0.0282%, about 0.0283%, about 0.0284%, about 0.0285%, about 0.0286%, about 0.0287%, about 0.0288%, about 0.0289%, about 0.029%, about 0.0291%, about 0.0292%, about 0.0293%, about 0.0294%, about 0.0295%, about 0.0296%, about 0.0297%, about 0.0298%, about 0.0299%, about 0.03%, about 0.0301%, about 0.0302%, about 0.0303%, about 0.0304%, about 0.0305%, about 0.0306%, about 0.0307%, about 0.0308%, about 0.0309%, about 0.031%, about 0.0311%, about 0.0312%, about 0.0313%, about 0.0314%, about 0.0315%, about 0.0316%, about 0.0317%, about 0.0318%, about 0.0319%, about 0.032%, about 0.0321%, about 0.0322%, about 0.0323%, about 0.0324%, about 0.0325%, about 0.0326%, about 0.0327%, about 0.0328%, about 0.0329%, about 0.033%, about 0.0331%, about 0.0332%, about 0.0333%, about 0.0334%, about 0.0335%, about 0.0336%, about 0.0337%, about 0.0338%, about 0.0339%, about 0.034%, about 0.0341%, about 0.0342%, about 0.0343%, about 0.0344%, about 0.0345%, about 0.0346%, about 0.0347%, about 0.0348%, about 0.0349%, about 0.035%, about 0.036%, about 0.037%, about 0.038%, about 0.039%, about 0.04%, about 0.041%, about 0.042%, about 0.043%, about 0.044%, about 0.045%, about 0.046%, about 0.047%, about 0.048%, about 0.049%, or about 0.050%, w/w % of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and optionally further including sodium phosphate dibasic; sodium dihydrogen phosphate monohydrate; disodium EDTA; methylparaben; and/or propylparaben.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and a propellant, and optionally further including sodium phosphate dibasic; sodium dihydrogen phosphate monohydrate; disodium EDTA; methylparaben; and/or propylparaben; and a propellant; wherein the propellant can be DME; A17; A31; AP35; A46; A48; A70; or AP70.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and a propellant, and optionally further including sodium phosphate dibasic; sodium dihydrogen phosphate monohydrate; disodium EDTA; methylparaben; and/or propylparaben; wherein the propellant is DME or AP35.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and a propellant, and optionally further including sodium phosphate dibasic; sodium dihydrogen phosphate monohydrate; disodium EDTA; methylparaben; and/or propylparaben; wherein the propellant is AP35.

In some embodiments, a pharmaceutical rectal foam composition comprises, consists essentially of, or consists of: a peptide, or a pharmaceutically acceptable salt, hydrate or solvate thereof; said peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and a propellant, and optionally further including sodium phosphate dibasic; sodium dihydrogen phosphate monohydrate; disodium EDTA; methylparaben; and/or propylparaben; wherein the propellant is AP35 propellant; wherein the pharmaceutical rectal foam composition has an amount of purified water ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant, and optionally further including sodium phosphate dibasic; sodium dihydrogen phosphate monohydrate; disodium EDTA; methylparaben; and/or propylparaben; wherein the pharmaceutical rectal foam composition has an amount of propylene glycol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant, and optionally further including sodium phosphate dibasic; sodium dihydrogen phosphate monohydrate; disodium EDTA; methylparaben; and/or propylparaben; wherein the pharmaceutical rectal foam composition has an amount of sodium phosphate dibasic ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant, and optionally further including sodium phosphate dibasic; sodium dihydrogen phosphate monohydrate; disodium EDTA; methylparaben; and/or propylparaben; wherein the pharmaceutical rectal foam composition has an amount of sodium dihydrogen phosphate monohydrate ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant, and optionally further including sodium phosphate dibasic; sodium dihydrogen phosphate monohydrate; disodium EDTA; methylparaben; and/or propylparaben; wherein the pharmaceutical rectal foam composition has an amount of disodium EDTA ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant, and optionally further including sodium phosphate dibasic; sodium dihydrogen phosphate monohydrate; disodium EDTA; methylparaben; and/or propylparaben; wherein the pharmaceutical rectal foam composition has an amount of methylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; wherein the pharmaceutical rectal foam composition has an amount of light mineral oil ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; wherein the pharmaceutical rectal foam composition has an amount of isopropyl myristate ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; wherein the pharmaceutical rectal foam composition has an amount of white petrolatum ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; wherein the pharmaceutical rectal foam composition has an amount of polyoxyl 20 cetostearyl ether ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; wherein the pharmaceutical rectal foam composition has an amount of cetyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; wherein the pharmaceutical rectal foam composition has an amount of stearyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; wherein the pharmaceutical rectal foam composition has an amount of propylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; wherein the pharmaceutical rectal foam composition has an amount of AP35 propellant ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition of the present disclosure can comprise a peptide of the present disclosure in an amount ranging from about 0.005% to about 0.040%; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; said composition comprising purified water in an amount ranging from about 50.0% to about 80.0%; propylene glycol in an amount ranging from about 8.0% to about 11.0%; sodium phosphate dibasic in an amount ranging from about 0.05% to about 0.20%; sodium dihydrogen phosphate monohydrate in an amount ranging from about 0.05% to about 0.20%; disodium EDTA in an amount ranging from about 0.02% to about 0.07%; methylparaben in an amount ranging from about 0.07% to about 0.30%; light mineral oil in an amount ranging from about 4.0% to about 7.0%; isopropyl myristate in an amount ranging from about 0.2% to about 0.7%; white petrolatum in an amount ranging from about 0.8% to about 1.5%; polyoxyl 20 cetostearyl ether in an amount ranging from about 2.0% to about 3.0%; cetyl alcohol in an amount ranging from about 0.7% to about 1.5%; stearyl alcohol in an amount ranging from about 0.7% to about 1.5%; propylparaben in an amount ranging from about 0.008% to about 0.050%; and AP35 propellant in an amount ranging from about 8.0% to about 12%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount ranging from about 0.008% to about 0.028%; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; said composition comprising purified water in an amount ranging from about 69.0% to about 78.0%; propylene glycol in an amount ranging from about 8.5% to about 10.5%; sodium phosphate dibasic in an amount ranging from about 0.14% to about 0.17%; sodium dihydrogen phosphate monohydrate in an amount ranging from about 0.10% to about 0.12%; disodium EDTA in an amount ranging from about 0.04% to about 0.06%; methylparaben in an amount ranging from about 0.08% to about 0.12%; light mineral oil in an amount ranging from about 5.0% to about 6.2%; isopropyl myristate in an amount ranging from about 0.4% to about 0.6%; white petrolatum in an amount ranging from about 0.9% to about 1.1%; polyoxyl 20 cetostearyl ether in an amount ranging from about 2.2% to about 2.6%; cetyl alcohol in an amount ranging from about 0.8% to about 1.1%; stearyl alcohol in an amount ranging from about 0.8% to about 1.1%; propylparaben in an amount ranging from about 0.01% to about 0.03%; and AP35 propellant in an amount ranging from about 9.0% to about 11.0%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount ranging from about 0.0087% to about 0.02883%; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; said composition comprising purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; propylparaben in an amount ranging from about 0.0180% to about 0.0200%; and AP35 propellant in an amount ranging from about 9.8835% to about 9.8838%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.0087%; and purified water; propylene glycol; light mineral oil; isopropyl myristate; white petrolatum; polyoxyl 20 cetostearyl ether; cetyl alcohol and/or stearyl alcohol; and AP35 propellant; and optionally further including sodium phosphate dibasic; sodium dihydrogen phosphate monohydrate; disodium EDTA; methylparaben; and/or propylparaben; said composition comprising purified water in an amount that is about 69.8761%; propylene glycol in an amount that is about 9.0116%; sodium phosphate dibasic in an amount that is about 0.1478%; sodium dihydrogen phosphate monohydrate in an amount that is about 0.1050%; disodium EDTA in an amount that is about 0.0451%; methylparaben in an amount that is about 0.0901%; light mineral oil in an amount that is about 5.4070%; isopropyl myristate in an amount that is about 0.4506%; white petrolatum in an amount that is about 0.9012%; polyoxyl 20 cetostearyl ether in an amount that is about 2.2529%; cetyl alcohol in an amount that is about 0.9012%; stearyl alcohol in an amount that is about 0.9012%; propylparaben in an amount that is about 0.0180%; and AP35 propellant in an amount that is about 9.8837%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.00961%; purified water in an amount that is about 77.5399%; propylene glycol in an amount that is about 10.0000%; sodium phosphate dibasic in an amount that is about 0.1640%; sodium dihydrogen phosphate monohydrate in an amount that is about 0.1165%; disodium EDTA in an amount that is about 0.0500%; methylparaben in an amount that is about 0.1000%; light mineral oil in an amount that is about 6.0000%; isopropyl myristate in an amount that is about 0.5000%; white petrolatum in an amount that is about 1.0000%; polyoxyl 20 cetostearyl ether in an amount that is about 2.5000%; cetyl alcohol in an amount that is about 1.0000%; stearyl alcohol in an amount that is about 1.0000%; propylparaben in an amount that is about 0.0200%; and AP35 propellant in an amount that is about 9.8837%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.0260%; purified water in an amount that is about 69.8587%; propylene glycol in an amount that is about 9.0116%; sodium phosphate dibasic in an amount that is about 0.1478%; sodium dihydrogen phosphate monohydrate in an amount that is about 0.1050%; disodium EDTA in an amount that is about 0.0451%; methylparaben in an amount that is about 0.0901%; light mineral oil in an amount that is about 5.4070%; isopropyl myristate in an amount that is about 0.4506%; white petrolatum in an amount that is about 0.9012%; polyoxyl 20 cetostearyl ether in an amount that is about 2.2529%; cetyl alcohol in an amount that is about 0.9012%; stearyl alcohol in an amount that is about 0.9012%; propylparaben in an amount that is about 0.0180%; and AP35 propellant in an amount that is about 9.8837%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.02883%; purified water in an amount that is about 77.5207%; propylene glycol in an amount that is about 10.0000%; sodium phosphate dibasic in an amount that is about 0.1640%; sodium dihydrogen phosphate monohydrate in an amount that is about 0.1165%; disodium EDTA in an amount that is about 0.0500%; methylparaben in an amount that is about 0.1000%; light mineral oil in an amount that is about 6.0000%; isopropyl myristate in an amount that is about 0.5000%; white petrolatum in an amount that is about 1.0000%; polyoxyl 20 cetostearyl ether in an amount that is about 2.5000%; cetyl alcohol in an amount that is about 1.0000%; stearyl alcohol in an amount that is about 1.0000%; propylparaben in an amount that is about 0.0200%; and AP35 propellant in an amount that is about 9.8837%, w/w % of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and a propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and a propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the propellant can be DME; A17; A31; AP35; A46; A48; A70; or AP70.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and a propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the propellant is DME or AP35.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and a propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the propellant is AP35.

In some embodiments, a pharmaceutical rectal foam composition comprises, consists essentially of, or consists of: a peptide, or a pharmaceutically acceptable salt, hydrate or solvate thereof; said peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of purified water ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of propylene glycol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of sodium phosphate dibasic ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of sodium dihydrogen phosphate monohydrate ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of disodium EDTA ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of methylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of light mineral oil ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of isopropyl myristate ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of white petrolatum ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of polyoxyl 20 cetostearyl ether ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some preferred embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of polyoxyl 20 cetostearyl ether ranging from about 1% to about 99%; from about 2% to about 98%; from about 3% to about 97%; from about 4% to about 96%; or from about 5% to about 95% w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of cetyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some preferred embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of cetyl alcohol ranging from about 0.5% to about 99.5; from about 0.6% to about 99.4%; from about 0.7% to about 99.3%; from about 0.8% to about 99.2%; from about 0.9% to about 99.1%; from about 1% to about 99%; from about 1.1% to about 98.9%; from about 1.2% to about 98.8%; from about 1.3% to about 98.7%; from about 1.4% to about 98.6%; from about 1.5% to about 98.5%; from about 1.6% to about 98.4%; from about 1.7% to about 98.3%; from about 1.8% to about 98.2%; from about 1.9% to about 98.1%; or from about 2% to about 98% w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of stearyl alcohol ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some preferred embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of stearyl alcohol ranging from about 0.5% to about 99.5; from about 0.6% to about 99.4%; from about 0.7% to about 99.3%; from about 0.8% to about 99.2%; from about 0.9% to about 99.1%; from about 1% to about 99%; from about 1.1% to about 98.9%; from about 1.2% to about 98.8%; from about 1.3% to about 98.7%; from about 1.4% to about 98.6%; from about 1.5% to about 98.5%; from about 1.6% to about 98.4%; from about 1.7% to about 98.3%; from about 1.8% to about 98.2%; from about 1.9% to about 98.1%; or from about 2% to about 98% w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of propylparaben ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of AP35 propellant ranging from about 0.000001% w/w to about 99.99999% w/w of the total composition, or from about 0.01% to about 99.99%; from about 0.02% to about 99.98%; from about 0.03% to about 99.97%; from about 0.04% to about 99.96%; from about 0.05% to about 99.95; from about 0.06% to about 99.94%; from about 0.07% to about 99.93%; from about 0.08% to about 99.92%; from about 0.09% to about 99.91%; from about 1% to about 99%; from about 2% to about 98%; from about 3% to about 97%; from about 4% to about 96%; from about 5% to about 95%; from about 6% to about 94%; from about 7% to about 93%; from about 8% to about 92%; from about 9% to about 91%; from about 10% to about 90%; from about 11% to about 89%; from about 12% to about 88%; from about 13% to about 87%; from about 14% to about 86%; from about 15% to about 85%; from about 16% to about 84%; from about 17% to about 83%; from about 18% to about 82%; from about 19% to about 81%; from about 20% to about 80%; from about 21% to about 79%; from about 22% to about 78%; from about 23% to about 77%; from about 24% to about 76%; from about 25% to about 75%; from about 26% to about 74%; from about 27% to about 73%; from about 28% to about 72%; from about 29% to about 71%; from about 30% to about 70%; from about 31% to about 69%; from about 32% to about 68%; from about 33% to about 67%; from about 34% to about 66%; from about 35% to about 65%; from about 36% to about 64%; from about 37% to about 63%; from about 38% to about 62%; from about 39% to about 61%; from about 40% to about 60%; from about 41% to about 59%; from about 42% to about 58%; from about 43% to about 57%; from about 44% to about 56%; from about 45% to about 55%; from about 46% to about 54%; from about 47% to about 53%; from about 48% to about 52%; from about 49% to about 51%; from about 50% to about 50%; from about 51% to about 49%; from about 52% to about 48%; from about 53% to about 47%; from about 54% to about 46%; from about 55% to about 45%; from about 56% to about 44%; from about 57% to about 43%; from about 58% to about 42%; from about 59% to about 41%; from about 60% to about 40%; from about 61% to about 39%; from about 62% to about 38%; from about 63% to about 37%; from about 64% to about 36%; from about 65% to about 35%; from about 66% to about 34%; from about 67% to about 33%; from about 68% to about 32%; from about 69% to about 31%; from about 70% to about 30%; from about 71% to about 29%; from about 72% to about 28%; from about 73% to about 27%; from about 74% to about 26%; from about 75% to about 25%; from about 76% to about 24%; from about 77% to about 23%; from about 78% to about 22%; from about 79% to about 21%; from about 80% to about 20%; from about 81% to about 19%; from about 82% to about 18%; from about 83% to about 17%; from about 84% to about 16%; from about 85% to about 15%; from about 86% to about 14%; from about 87% to about 13%; from about 88% to about 12%; from about 89% to about 11%; from about 90% to about 10%; from about 91% to about 9%; from about 92% to about 8%; from about 93% to about 7%; from about 94% to about 6%; from about 95% to about 5%; from about 96% to about 4%; from about 97% to about 3%; from about 98% to about 2%; from about 99% to about 1%; from about 99.91 to about 0.09%; from about 99.92 to about 0.08%; from about 99.93 to about 0.07%; from about 99.94 to about 0.06%; from about 99.95 to about 0.05%; from about 99.96 to about 0.04%; from about 99.97 to about 0.03%; from about 99.98 to about 0.02%; or from about 99.99 to about 0.01%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of AP35 propellant ranging from about 8% to about 92%; from about 9% to about 91%; or from about 10% to about 90%, w/w of the total composition.

In some embodiments, a pharmaceutical rectal foam composition comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; and polyoxyl 20 cetostearyl ether; cetyl alcohol and/or stearyl alcohol; and AP35 propellant; and optionally further comprising purified water; propylene glycol; light mineral oil; isopropyl myristate; white petrolatum; sodium phosphate dibasic; sodium dihydrogen phosphate monohydrate; disodium EDTA; methylparaben; and/or propylparaben; wherein the pharmaceutical rectal foam composition has an amount of AP35 propellant ranging from about 8% to about 92%; from about 8.1% to about 91.9%; from about 8.2% to about 91.8%; from about 8.3% to about 91.7%; from about 8.4% to about 91.6%; from about 8.5% to about 91.5%; from about 8.6% to about 91.4%; from about 8.7% to about 91.3%; from about 8.8% to about 91.2%; from about 8.9% to about 91.1%; from about 9% to about 91%; from about 9.1% to about 90.9%; from about 9.2% to about 90.8%; from about 9.3% to about 90.7%; from about 9.4% to about 90.6%; from about 9.5% to about 90.5%; from about 9.6% to about 90.4%; from about 9.7% to about 90.3%; from about 9.8% to about 90.2%; from about 9.9% to about 90.1%; or from about 10% to about 90%, w/w of the total composition.

In some embodiments, a pharmaceutical rectal foam composition of the present disclosure can comprise a peptide of the present disclosure in an amount ranging from about 0.005% to about 0.040%; and polyoxyl 20 cetostearyl ether in an amount ranging from about 2.0% to about 3.0%; cetyl alcohol in an amount ranging from about 0.7% to about 1.5%; stearyl alcohol in an amount ranging from about 0.7% to about 1.5%; and AP35 propellant in an amount ranging from about 8.0% to about 12%, w/w % of the total composition.

In some embodiments, a pharmaceutical rectal foam composition of the present disclosure can comprise a peptide of the present disclosure in an amount ranging from about 0.005% to about 0.040%; and polyoxyl 20 cetostearyl ether in an amount ranging from about 2.0% to about 3.0%; cetyl alcohol in an amount ranging from about 0.7% to about 1.5%; stearyl alcohol in an amount ranging from about 0.7% to about 1.5%; and AP35 propellant in an amount ranging from about 8.0% to about 12%, w/w % of the total composition; and further optionally comprise purified water in an amount ranging from about 50.0% to about 80.0%; propylene glycol in an amount ranging from about 8.0% to about 11.0%; sodium phosphate dibasic in an amount ranging from about 0.05% to about 0.20%; sodium dihydrogen phosphate monohydrate in an amount ranging from about 0.05% to about 0.20%; disodium EDTA in an amount ranging from about 0.02% to about 0.07%; methylparaben in an amount ranging from about 0.07% to about 0.30%; light mineral oil in an amount ranging from about 4.0% to about 7.0%; isopropyl myristate in an amount ranging from about 0.2% to about 0.7%; white petrolatum in an amount ranging from about 0.8% to about 1.5%; propylparaben in an amount ranging from about 0.008% to about 0.050%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount ranging from about 0.008% to about 0.028%; polyoxyl 20 cetostearyl ether in an amount ranging from about 2.2% to about 2.6%; cetyl alcohol in an amount ranging from about 0.8% to about 1.1%; stearyl alcohol in an amount ranging from about 0.8% to about 1.1%; and AP35 propellant in an amount ranging from about 9.0% to about 11.0%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁o Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount ranging from about 0.008% to about 0.028%; polyoxyl 20 cetostearyl ether in an amount ranging from about 2.2% to about 2.6%; cetyl alcohol in an amount ranging from about 0.8% to about 1.1%; stearyl alcohol in an amount ranging from about 0.8% to about 1.1%; and AP35 propellant in an amount ranging from about 9.0% to about 11.0%, w/w % of the total composition; and further optionally comprise purified water in an amount ranging from about 69.0% to about 78.0%; propylene glycol in an amount ranging from about 8.5% to about 10.5%; sodium phosphate dibasic in an amount ranging from about 0.14% to about 0.17%; sodium dihydrogen phosphate monohydrate in an amount ranging from about 0.10% to about 0.12%; disodium EDTA in an amount ranging from about 0.04% to about 0.06%; methylparaben in an amount ranging from about 0.08% to about 0.12%; light mineral oil in an amount ranging from about 5.0% to about 6.2%; isopropyl myristate in an amount ranging from about 0.4% to about 0.6%; white petrolatum in an amount ranging from about 0.9% to about 1.1%; propylparaben in an amount ranging from about 0.01% to about 0.03%; w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount ranging from about 0.0087% to about 0.02883%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and AP35 propellant in an amount ranging from about 9.8835% to about 9.8838%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount ranging from about 0.0087% to about 0.02883%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and AP35 propellant in an amount ranging from about 9.8835% to about 9.8838%, w/w % of the total composition; and further optionally comprise purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; propylparaben in an amount ranging from about 0.0180% to about 0.0200%; w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.0087%; polyoxyl 20 cetostearyl ether in an amount that is about 2.2529%; cetyl alcohol in an amount that is about 0.9012%; stearyl alcohol in an amount that is about 0.9012%; and AP35 propellant in an amount that is about 9.8837%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.0087%; polyoxyl 20 cetostearyl ether in an amount that is about 2.2529%; cetyl alcohol in an amount that is about 0.9012%; stearyl alcohol in an amount that is about 0.9012%; and AP35 propellant in an amount that is about 9.8837%, w/w % of the total composition; and further optionally comprise purified water in an amount that is about 69.8761%; propylene glycol in an amount that is about 9.0116%; sodium phosphate dibasic in an amount that is about 0.1478%; sodium dihydrogen phosphate monohydrate in an amount that is about 0.1050%; disodium EDTA in an amount that is about 0.0451%; methylparaben in an amount that is about 0.0901%; light mineral oil in an amount that is about 5.4070%; isopropyl myristate in an amount that is about 0.4506%; white petrolatum in an amount that is about 0.9012%; propylparaben in an amount that is about 0.0180%; w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.00961%; polyoxyl 20 cetostearyl ether in an amount that is about 2.5000%; cetyl alcohol in an amount that is about 1.0000%; stearyl alcohol in an amount that is about 1.0000%; and AP35 propellant in an amount that is about 9.8837%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.00961%; polyoxyl 20 cetostearyl ether in an amount that is about 2.5000%; cetyl alcohol in an amount that is about 1.0000%; stearyl alcohol in an amount that is about 1.0000%; and AP35 propellant in an amount that is about 9.8837%, w/w % of the total composition; and optionally further comprise purified water in an amount that is about 77.5399%; propylene glycol in an amount that is about 10.0000%; sodium phosphate dibasic in an amount that is about 0.1640%; sodium dihydrogen phosphate monohydrate in an amount that is about 0.1165%; disodium EDTA in an amount that is about 0.0500%; methylparaben in an amount that is about 0.1000%; light mineral oil in an amount that is about 6.0000%; isopropyl myristate in an amount that is about 0.5000%; white petrolatum in an amount that is about 1.0000%; and propylparaben in an amount that is about 0.0200%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.0260%; polyoxyl 20 cetostearyl ether in an amount that is about 2.2529%; cetyl alcohol in an amount that is about 0.9012%; stearyl alcohol in an amount that is about 0.9012%; and AP35 propellant in an amount that is about 9.8837%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.0260%; polyoxyl 20 cetostearyl ether in an amount that is about 2.2529%; cetyl alcohol in an amount that is about 0.9012%; stearyl alcohol in an amount that is about 0.9012%; and AP35 propellant in an amount that is about 9.8837%, w/w % of the total composition; and further optionally comprise purified water in an amount that is about 69.8587%; propylene glycol in an amount that is about 9.0116%; sodium phosphate dibasic in an amount that is about 0.1478%; sodium dihydrogen phosphate monohydrate in an amount that is about 0.1050%; disodium EDTA in an amount that is about 0.0451%; methylparaben in an amount that is about 0.0901%; light mineral oil in an amount that is about 5.4070%; isopropyl myristate in an amount that is about 0.4506%; white petrolatum in an amount that is about 0.9012%; and propylparaben in an amount that is about 0.0180% w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.02883%; polyoxyl 20 cetostearyl ether in an amount that is about 2.5000%; cetyl alcohol in an amount that is about 1.0000%; stearyl alcohol in an amount that is about 1.0000%; and AP35 propellant in an amount that is about 9.8837%, w/w % of the total composition.

In some embodiments, a pharmaceutical rectal foam composition of the present disclosure comprises, consists essentially of, or consists of: a peptide, or a pharmaceutically acceptable salt, hydrate or solvate thereof, comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated, in an amount that is about 0.02883%; polyoxyl 20 cetostearyl ether in an amount that is about 2.5000%; cetyl alcohol in an amount that is about 1.0000%; stearyl alcohol in an amount that is about 1.0000%; and AP35 propellant in an amount that is about 9.8837%, w/w % of the total composition; and optionally further comprise purified water in an amount that is about 77.5207%; propylene glycol in an amount that is about 10.0000%; sodium phosphate dibasic in an amount that is about 0.1640%; sodium dihydrogen phosphate monohydrate in an amount that is about 0.1165%; disodium EDTA in an amount that is about 0.0500%; methylparaben in an amount that is about 0.1000%; light mineral oil in an amount that is about 6.0000%; isopropyl myristate in an amount that is about 0.5000%; white petrolatum in an amount that is about 1.0000%; and propylparaben in an amount that is about 0.0200% w/w % of the total composition.

### Canisters and Other Pressurized Gas Containers

In some embodiments, a pharmaceutical composition of the present disclosure, formulated as a foam (e.g., a pharmaceutical rectal foam composition), can be provided in a container, e.g., a pressurized gas container. For example, in some embodiments, without limitation, the pharmaceutical composition formulated as a foam (e.g., a pharmaceutical rectal foam composition) can be provided in a canister, e.g., an aerosol canister.

Thus, in some embodiments, a canister of the present disclosure comprises a pharmaceutical composition formulated as a foam (e.g., a pharmaceutical rectal foam composition).

As used herein, an “aerosol” is a pressurized dosage form containing one or more active ingredients (e.g., a peptide of the present disclosure), and which upon actuation emits a dispersion of liquid and/or solid materials in a gaseous medium. In some embodiments, the dosage form is packaged under pressure in a suitable container equipped with a valve assembly. In some embodiments, when the valve is opened, the internal pressure within the container forces the aerosol out the valve.

In some embodiments, a canister of the present disclosure is suitable for accommodating a pressurized product and comprises an outlet capable of releasing a foam.

In some embodiments, a canister of the present disclosure is suitable for accommodating a pressurized product and comprises an outlet capable of releasing a foam; wherein the outlet is a valve.

In some embodiments, a suitable pressurized gas container for the purposes of dispensing the pharmaceutical rectal foam composition of the present disclosure can be a cylindrical vessel comprising: metal (e.g., aluminum, tinplate, and the like), protected or shatter resistant glass, plastic, or shatter-proof glass. Those having ordinary skill in the art will recognize that the choice of pressurized gas container may depend on one or more factors, e.g., without limitation, the compressive strength and/or breaking strength, corrosion resistance, ease of filling, ease of sterilizing, aesthetic aspects, handle-ability, printing properties, and the like.

In some embodiments, a pressurized gas container of the present disclosure is a canister.

In some embodiments, a canister of the present disclosure comprises aluminum.

In some embodiments, for reasons of corrosion protection, the interior of metal canisters can be coated. Corrosion-resistant coatings are known to those having ordinary skill in the art, and can include, e.g., polyester, epoxyphenol and polyamide-imide coatings; film laminates made of polyethylene (PE), polypropylene (PP) and/or polyethylene terephthalate (PET) and other corrosion-resistant coatings known in the art.

In some embodiments, a pressurized gas container of the present disclosure (e.g., a canister) can be single-part or two-part, or three-part cylindrical, conical or differently shaped containers. If plastics are used as the container material, then these plastics should be resistant to chemicals and the sterilization temperature, gas-tight, impact-resistant and stable to internal pressures.

In some embodiments, a pressurized gas container of the present disclosure (e.g., a canister) can comprise a valve. Valves which are advantageous according to the present disclosure are known by those having ordinary skill in the art.

In some embodiments, a pressurized gas container of the present disclosure (e.g., a canister) comprises a spray head. Advantageous spray heads for the purposes of the present disclosure are, for example, foaming heads for upright use (e.g., when holding the canister vertically) or foam heads for overhead application using one or more channels.

In some embodiments, a pressurized gas container of the present disclosure (e.g., a canister) comprises one or more propellants, e.g., one or more propellants as described herein, in addition the peptide of the present disclosure. Briefly, suitable propellants are described herein, and can include readily volatile, liquefied propellant gases, such as, e.g., dimethyl ether (DME) and/or linear or branched-chain hydrocarbons with two to five carbon atoms (such as, in particular, ethane, propane, butane, isobutane and/or pentane), which can be used on their own or in a mixture with one another. Exemplary propellants are described herein.

In some embodiments, a pressurized gas container of the present disclosure (e.g., a canister) comprises compressed air, and also other gases which are under pressure, such as air, oxygen, nitrogen, hydrogen, helium, krypton, xenon, radon, argon, nitrous oxide and carbon dioxide. In some embodiments, said gases can in each case be used individually or in any desired mixtures with one another.

In some embodiments, a pressurized gas container of the present disclosure (e.g., a canister) comprises a pharmaceutical rectal foam composition, wherein the foam is dispensed or provided as a finely creamy and/or rich foam. In some embodiments, a pressurized gas container of the present disclosure (e.g., a canister) can provide finely creamy and/or rich foams when the pharmaceutical rectal foam composition comprises linear or branched-chain, halogenated or nonhalogenated hydrocarbons and/or one or more gases selected from carbon dioxide, oxygen, compressed air and/or nitrogen.

In some embodiments, a pressurized gas container of the present disclosure comprises a canister.

In some embodiments, a pressurized gas container of the present disclosure comprises a canister and a valve.

In some embodiments, a pressurized gas container of the present disclosure comprises a canister, a valve, and a foam shield.

In some embodiments, a pressurized gas container of the present disclosure comprises a canister, a valve, and a foam shield, wherein the pharmaceutical rectal foam composition contained therein is presented as a multidose unit.

In some embodiments, a pressurized gas container of the present disclosure comprises an aluminum aerosol canister.

In yet other embodiments, a pressurized gas container of the present disclosure comprises an aluminum aerosol canister, a valve, a foam shield, and a metered head actuator.

In some other embodiments, a pressurized gas container of the present disclosure comprises an aluminum aerosol canister, fitted with a 1-inch valve, a foam shield, and a 1.35 mL metered head actuator.

In some embodiments, a pharmaceutical rectal foam composition is packaged in aerosol canisters which are crimped with a valve, pressurized with propellant and equipped with an actuator suitable for foam dispensing. In yet other embodiments, a metered dosage unit can utilized, to achieve delivery of repeatable measured doses of foam.

In some embodiments, a pharmaceutical rectal foam composition is filled into an aerosol canister equipped with a metered dose valve. In the hands of the patient the a pharmaceutical rectal foam composition is dispensed via an actuator adapted to direct the dose from the valve to the patient.

In some embodiments, a pharmaceutical rectal foam composition can be prepared by combining (i) the peptide of the present disclosure in an amount sufficient to provide a plurality of therapeutically effective doses; (ii) a fluid, e.g., a propellant, in an amount sufficient to propel a plurality of doses, e.g., from an aerosol canister; (iii) optionally, one or more excipients. In some embodiments, the components can be dispersed using a conventional mixer or homogenizer, by shaking, or by ultrasonic energy as well as by the use of a bead mill or a microfluidizer.

In some embodiments, bulk formulations can be transferred to smaller individual aerosol vials by using valve to valve transfer methods, pressure filling or by using conventional cold-fill methods.

Aerosol canisters equipped with conventional valves, preferably metered dose valves, can be used to deliver the pharmaceutical rectal foam of the present disclosure. In some embodiments, selection of appropriate valve assemblies for use with aerosol formulations is dependent upon the particular component and other adjuvants used (if any), on the fluid, e.g., propellant, and on the particular drug being used. Conventional neoprene and buna valve rubbers used in metered dose valves for delivering conventional CFC formulations often have less than optimal valve delivery characteristics and ease of operation when used with formulations containing HFC-134a (1,1,1,2-tetrafluoroethane) or HFC-227 (1,1,1,2,3,3,3-heptafluoropropane). Therefore, certain formulations of the invention are preferably dispensed via a valve assembly wherein the diaphragm is made of a nitrile rubber such as DB-218 (American Gasket and Rubber, Schiller Park, Ill.) or an EPDM rubber such as VISTALON synthetic rubber (Exxon), ROYALENE synthetic rubber (UniRoyal), bunaEP (Bayer). Also suitable are diaphragms fashioned by extrusion, injection, molding or compression molding from a thermoplastic elastomeric material, such as FLEXOMER GERS 1085 NT polyolefin (Union Carbide).

In some embodiments, conventional aerosol canisters, coated or uncoated, anodized or unanodized, e.g., those of aluminum, glass, stainless steel, polybutyl or polyethylene terephthalate, and coated canisters or cans with epon, epoxy, etc., can be used to contain a pharmaceutical rectal foam composition.

In some embodiments, a pressurized gas container of the present disclosure comprises an aluminum aerosol canister, wherein the canister can be individually packaged in a carton and/or kit.

In some embodiments, a canister of the present disclosure can comprise a minimum of about 20.0, 20.2, 20.4, 20.6, 20.8, 30.0, 30.2, 30.4, 30.6, 30.8, 31.0, 31.2, 31.4, 31.6, 31.8, 32.0, 35, or 45 grams of product, and about 2.0, 2.5, 2.6, 2.8, 3.0, 3.2, 3.4, 3.6, 3.8, 4, or 4.5 grams of propellant.

Exemplary methods of preparing pressurized gas containers (e.g., aerosol canisters) are provided in U.S. Pat. Nos. 2,968,628; 3,966,090; 3,970,219; 4,440,320; 6,875,438; 8,006,873; 9,668,972; 10,265,404; and 10,906,729; the disclosures of which are incorporated herein by reference in their entireties.

Any of the peptides of the present disclosure, and/or any of the pharmaceutical rectal foam compositions of the present disclosure, can be provided in a pressurized gas container, e.g., a canister. For example, a peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃ are connected by disulfide bonds; and wherein Cth₂ and Cys₁₀ are connected by a thioether bond, or a pharmaceutically acceptable salt, hydrate or solvate thereof, or a pharmaceutical composition, unit dosage form, or pharmaceutical rectal foam composition comprising the same, can be provided in a pressurized gas container (e.g., a canister).

### Non-Pressurized Containers

In some embodiments, a pharmaceutical composition of the present disclosure may be formulated into products that can be dispensed as foams, which do not require the addition of a propellant.

In some embodiments, a pharmaceutical composition of the present disclosure may be formulated into products that can be dispensed as foams from a reservoir using a release assembly (e.g., a hand pump) whenever the release assembly is put into action. The amount of the foam dispensed by the pump may or may not be metered to dispense a consistent amount of the foam. Preferably, the amount is metered to deliver a specific dose.

Exemplary descriptions of foaming heads are provided in U.S. Pat. Nos. 5,443,369; 6,053,364; and 7,757,899, the disclosures of which are incorporated herein by reference in their entireties.

Exemplary descriptions of hand pumps, and other pumps useful in dispensing the pharmaceutical compositions of the present disclosure are provided in U.S. Pat. Nos. 6,547,162; 7,147,133; 7,726,518; 7,673,854; and 7,735,692; the disclosures of which are incorporated herein by reference in their entireties.

In some embodiments, the dispenser or pump head may include additional or altered features that assist in optimizing foam stability, especially for low-viscosity formulations. These features include, but are not limited to, the inclusion, arrangement, and hole size of meshes in the pump head; the size and shape of the mix chamber; and varied dip tube and nozzle lengths.

In some embodiments, a pharmaceutical composition of the present disclosure may be formulated as a foam, wherein the foam is generated through mechanical aeration. For example, in some embodiments, a method for producing a foam of the present invention comprises: providing a dispenser comprising a reservoir operably linked (e.g., in fluid communication) with a release assembly (e.g., dispensing head); filling the reservoir of the dispensing system with a foamable formulation; and actuating the release assembly to manually aerate the formulation, thereby releasing a foam from the release assembly.

In some embodiments, a pharmaceutical composition of the present disclosure may be formulated into products that can be dispensed as foams, which do not require the addition of a propellant (e.g., a non-aerosol formulation).

In some embodiments, a non-aerosol formulation may be contained in a non-aerosol dispenser equipped with a conventional hand pump, and the composition may be pumped onto the hands or other areas of the body. The pumping action required to dispense the pharmaceutical compositions will create a discrete volume of a dispensed composition as a stable foam.

Any of the peptides of the present disclosure, and/or any of the pharmaceutical rectal foam compositions of the present disclosure, can be provided in a non-pressurized container, e.g., as a non-aerosol formulation. For example, a peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃ are connected by disulfide bonds; and wherein Cth₂ and Cys₁₀ are connected by a thioether bond, or a pharmaceutically acceptable salt, hydrate or solvate thereof, or a pharmaceutical composition, unit dosage form, or pharmaceutical rectal foam composition comprising the same, can be provided in a non-pressurized container.

### Kits

The present disclosure includes a variety of kits for conveniently and/or effectively carrying out methods of the present disclosure. Typically, kits will comprise sufficient amounts and/or numbers of components to allow a user to perform one or multiple treatments of a subject(s) and/or to perform one or multiple experiments.

In some embodiments, the present disclosure provides kits for treating, limiting the symptoms of, reducing the severity of, or relieving the pain associated with visceral pain conditions of the abdominal region, e.g., including without limitation, bladder pain associated with interstitial cystitis/bladder pain syndrome (IC/BPS), and endometriosis pain.

In one embodiment, the present disclosure provides kits for treating, limiting the symptoms of, reducing the severity of, or relieving the pain associated with, interstitial cystitis/bladder pain syndrome (IC/BPS), in a subject in need thereof; wherein the pain associated with IC/BPS is selected from: urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; or any combination thereof.

In some embodiments, a kit may comprise packaging and instructions and/or a delivery agent to form a pharmaceutical composition of the present disclosure. The delivery agent may comprise, for example, one or more excipients.

In some embodiments, a kit may comprise packaging and instructions and/or a device or delivery means to dispense a pharmaceutical composition of the present disclosure. For example, in some embodiments, a kit may comprise packaging, instructions, and a pressurized gas container (e.g., a canister) comprising a peptide of the present disclosure.

In some embodiments, a kit comprises the means for preparing a reconstituted unit dose comprising, e.g., a lyophilized unit dose, and a pharmaceutically acceptable diluent for reconstitution.

In additional embodiments, assay screening kits are provided. In some embodiments, a kit includes a container for the screening assay. In other embodiments, an instruction for the use of the assay and the information about the screening method are to be included in the kit.

In some embodiments, a peptide of the present disclosure, and/or a pharmaceutical composition of the present disclosure, can be packaged into an article of manufacture (i.e., a kit) using containers, vials, or the like. For example, an article of manufacture can include (i) a peptide of the present disclosure; and (ii) one or more excipients (ii). In addition, an article of manufacture also can include instructions for use.

In some embodiments, a kit may further include a suitably aliquoted pharmaceutical composition, liquid pharmaceutical composition, or unit dosage form, and/or pharmaceutical rectal foam composition of the present disclosure. In addition, a pharmaceutical composition as described herein, and/or a peptide of the present disclosure, may be partially or wholly dehydrated or aqueous (where applicable). Kits contemplated herein may be stored at room temperatures or at refrigerated temperatures as disclosed herein depending on the particular formulation.

In some embodiments, the container means of the kits will generally include at least one vial, test tube, flask, bottle, syringe, canister, or other container means, into which a pharmaceutical composition may be placed, and preferably, suitably aliquoted. Where an additional component is provided, the kit will also generally contain one or more additional containers into which this agent or component may be placed. Kits herein will also typically include a means for containing the agent, composition and any other reagent containers in close confinement for commercial sale. Such containers may include injection or blow-molded plastic containers into which the desired vials or canisters are retained.

In some embodiments, the kit provides instructions for using the pharmaceutical composition of the kit, and/or one or more of the components of the kit. The instructions will generally include information about the use of the kit treatment of a subject in need thereof. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable excipient, e.g., a buffer. It can further include other materials desirable from a commercial and user standpoint, including other buffers, diluents, filters, needles, syringes, and package inserts with instructions for use.

In some embodiments, a kit contains a peptide of the present disclosure as provided as a unit dosage form, wherein the unit dosage form comprises a peptide of the present disclosure, or a pharmaceutically acceptable salt thereof; and one or more excipients.

In some embodiments, a kit comprises a pharmaceutical rectal foam composition comprising a peptide of the present disclosure, dissolved or suspended in a liquid carrier vehicle.

In some embodiments, a kit comprises a pharmaceutical rectal foam composition comprising a peptide of the present disclosure, dissolved or suspended in a liquid carrier vehicle, and a surfactant/emulsifier with foaming properties.

In some embodiments, a kit comprises a pharmaceutical rectal foam composition comprising a peptide of the present disclosure, dissolved or suspended in a liquid carrier vehicle, and a surfactant/emulsifier with foaming properties, and a propellant (e.g., a propellant gas).

In some embodiments, a unit dosage form is suitable for rectal administration. In some embodiments, unit dosage forms suitable for rectal administration include, without limitation, the following: enemas, rectal gels, rectal foams, rectal aerosols, suppositories, jelly suppositories, and enemas (e.g., retention enemas).

In some embodiments, unit dosage forms may be contained in a container e.g., without limitation, a paper or cardboard box, a glass or plastic bottle or jar, a re-sealable bag (for example, to hold a “refill” of tablets for placement into a different container), a blister pack with individual doses for pressing out of the pack according to a therapeutic schedule; or a pressurized gas container (e.g., rectal foam or rectal aerosol). It is feasible that more than one container can be used together in a single package to provide a single dosage form. For example, enemas or suppositories may be contained in a bottle which is in turn contained within a box. In some embodiments, the unit dosage forms are provided in a container further comprising a desiccant. In a further embodiment, the unit dosage forms are provided in a container, e.g., a bottle, jar or re-sealable bag, containing a desiccant. In a further embodiment, the container containing the unit dosage forms is packaged with administration or dosage instructions.

In some embodiments, enema formulations containing the peptides of the present disclosure, or pharmaceutical compositions thereof, can be provided in “ready-to-use” form.

In some embodiments, enema formulations containing the peptides of the present disclosure, or pharmaceutical compositions thereof, are provided in one or more kits or packs.

In certain embodiments, the kit or pack includes two or more separately contained/packaged components, e.g. two components, which, when mixed together, provide the desired formulation (e.g., as a suspension).

In some embodiments, the present disclosure provides a two component system that includes a first component and a second component, wherein: (i) the first component (e.g., contained in a sachet) includes the peptide of the present disclosure (as described anywhere herein), and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, it may be possible to ship a pharmaceutical composition of the present disclosure to the end-user as a kit comprising at least a first component comprising a peptide of the present disclosure, or a pharmaceutically acceptable salt thereof (A); and a second component comprising at least one pharmaceutically acceptable excipient (B). Further additives (D) may be a third separate component of the kit, or may be already mixed with components (A) and/or (B). The end-user may prepare the formulation for use by just adding one or more liquids (e.g., water and/or other pharmaceutically acceptable liquid as described herein) (C) to the components of the kit and mixing. In some embodiments, the components of the kit may also be formulations in water. Of course it is possible to combine an aqueous formulation of one of the components with a dry formulation of the other component(s). As an example, the kit can comprise one formulation of a peptide of the present disclosure, or a pharmaceutically acceptable salt thereof (A) and optionally water (C); and a second, separate formulation of at least one excipient (B), water as component (C) and other optional additional components (D). The concentrations of the components (A), (B), (C) and optionally (D) will be selected by the skilled artisan depending on the formulation and/or the technique to be used for treatment.

In some embodiments, each of the one or more liquids (C) is water, or a physiologically acceptable solvent, or a mixture of water and one or more physiologically acceptable solvents. Typical such solvents include, without limitation, water, glycerol, ethylene glycol, propylene glycol, polyethylene glycol and polypropylene glycol. In other embodiments, each of the one or more liquids is water. In other embodiments, each of the one or more liquids is an oil, e.g. natural and/or synthetic oils that are commonly used in pharmaceutical preparations.

In some embodiments, the pharmaceutically acceptable excipient (B) can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents. In some embodiments, the pharmaceutically acceptable excipients can be independently selected from thickeners, viscosity enhancing agents, mucoadhesive agents, buffers, preservatives, diluents, binders, lubricants, glidants, disintegrants, and fillers. In some embodiments, each of the pharmaceutically acceptable excipients can be independently selected from thickeners, viscosity enhancing agents, bulking agents, mucoadhesive agents, buffers, preservatives, and fillers. In some embodiments, each of the pharmaceutically acceptable excipients can be independently selected from diluents, binders, lubricants, glidants, and disintegrants.

Exemplary diluents, fillers, bulking agents, buffers, binders disintegrants, glidants, lubricants, thickeners, viscosity enhancing agents, preservatives, and mucoadhesive agents, that can be included in a kit are described herein.

In some embodiments, a pharmaceutical rectal foam composition can be supplied in a kit comprising a metered-dose canister containing enough rectal foam for 22 actuations, e.g., 1 priming actuation followed by 21 dosing actuations. In some embodiments, each canister will be provided with a kit comprising 21 prelubricated applicators and disposal bags for used applicators.

In some embodiments, a kit comprises a can, e.g., a 30 g 35 mm×65 mm can; a valve, e.g., a CV/1″ Nitrile AR/PP; an actuator, e.g., a metered head 1.35 mL actuator; a foam shield, Lablabo; aeropin 35; SHP; DIC; PAD; and SHPR LBL.

Any of the peptides of the present disclosure, or a pharmaceutical composition thereof, can be provided in a kit. For example, a peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃ are connected by disulfide bonds; and wherein Cth₂ and Cys₁₀ are connected by a thioether bond; or a pharmaceutically acceptable salt, hydrate or solvate thereof, or a combination, mixture, or composition comprising the same; can be provided in a kit contemplated by the present disclosure to treat a visceral pain condition in a subject in need thereof.

### Long-Term Stability Compositions

Any of the pharmaceutical compositions of the present disclosure can be formulated as a compositions having long-term stability.

In some embodiments, a pharmaceutical composition comprising, consisting essentially of, or consisting of a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; and an excipient; can be stable at about −20° C.; about −19° C.; about −18° C.; about −17° C.; about −16° C.; about −15° C.; about −14° C.; about −13° C.; about −12° C.; about −11° C.; about −10° C.; about −9° C.; about −8° C.; about −7° C.; about −6° C.; about −5° C.; about −4° C.; about −3° C.; about −2° C.; about −1° C.; about 0° C.; about 1° C.; about 2° C.; about 3° C.; about 4° C.; about 5° C.; about 6° C.; about 7° C.; about 8° C.; about 9° C.; about 10° C.; about 11° C.; about 12° C.; about 13° C.; about 14° C.; about 15° C.; about 16° C.; about 17° C.; about 18° C.; about 19° C.; about 20° C.; about 21° C.; about 22° C.; about 23° C.; about 24° C.; or about 25° C.; for about 1 month or more.

In some embodiments, a pharmaceutical composition comprising, consisting essentially of, or consisting of a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; and an excipient; can be stable at about −20° C. for about 0.5 months; about 1 months; about 1.5 months; about 2 months; about 2.5 months; about 3 months; about 3.5 months; about 4 months; about 4.5 months; about 5 months; about 5.5 months; about 6 months; about 6.5 months; about 7 months; about 7.5 months; about 8 months; about 8.5 months; about 9 months; about 9.5 months; about 10 months; about 10.5 months; about 11 months; about 11.5 months; about 12 months; about 12.5 months; about 13 months; about 13.5 months; about 14 months; about 14.5 months; about 15 months; about 15.5 months; about 16 months; about 16.5 months; about 17 months; about 17.5 months; about 18 months; about 18.5 months; about 19 months; about 19.5 months; about 20 months; about 20.5 months; about 21 months; about 21.5 months; about 22 months; about 22.5 months; about 23 months; about 23.5 months; about 24 months; about 24.5 months; about 25 months; about 25.5 months; about 26 months; about 26.5 months; about 27 months; about 27.5 months; about 28 months; about 28.5 months; about 29 months; about 29.5 months; about 30 months; about 30.5 months; about 31 months; about 31.5 months; about 32 months; about 32.5 months; about 33 months; about 33.5 months; about 34 months; about 34.5 months; about 35 months; about 35.5 months; about 36 months; about 36.5 months; about 37 months; about 37.5 months; about 38 months; about 38.5 months; about 39 months; about 39.5 months; about 40 months; about 40.5 months; about 41 months; about 41.5 months; about 42 months; about 42.5 months; about 43 months; about 43.5 months; about 44 months; about 44.5 months; about 45 months; about 45.5 months; about 46 months; about 46.5 months; about 47 months; about 47.5 months; about 48 months; about 48.5 months; about 49 months; about 49.5 months; or about 50 months.

In some embodiments, a pharmaceutical composition comprising, consisting essentially of, or consisting of a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; and an excipient; can be stable at about 1.5° C., about 2° C., about 2.5° C., about 3° C., about 3.5° C., about 4° C., about 4.5° C., about 5° C., about 5.5° C., about 6° C., about 6.5° C., about 7° C., about 7.5° C., about 8° C., about 8.5° C., or about 9° C.; for about 0.5 months; about 1 months; about 1.5 months; about 2 months; about 2.5 months; about 3 months; about 3.5 months; about 4 months; about 4.5 months; about 5 months; about 5.5 months; about 6 months; about 6.5 months; about 7 months; about 7.5 months; about 8 months; about 8.5 months; or about 9 months.

In some embodiments, a pharmaceutical composition comprising, consisting essentially of, or consisting of a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; and an excipient; can be stable at about 23° C., about 23.5° C., about 24° C., about 24.5° C., about 25° C., about 25.5° C., about 26° C., about 26.5° C., or about 27° C.; for about 0.1 months; about 0.2 months; about 0.3 months; about 0.4 months; about 0.5 months; about 0.6 months; about 0.7 months; about 0.8 months; about 0.9 months; about 1 months; about 1.1 months; about 1.2 months; about 1.3 months; about 1.4 months; or about 1.5 months.

In some embodiments, a pharmaceutical composition comprising, consisting essentially of, or consisting of a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Ac-Cys₁-Cth₂-Glu₃-Leu₄-Cys₅-Cys₆-Asn₇-Val₈-Ala₉-Cys₁₀-Tyr₁₁-Gly₁₂-Cys₁₃-OH or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; wherein Ac-indicates an acetylated N-terminus; and wherein-OH indicates an unmodified C-terminus, and an excipient; can be stable at about −20° C.; about −19° C.; about −18° C.; about −17° C.; about −16° C.; about −15° C.; about −14° C.; about −13° C.; about −12° C.; about −11° C.; about −10° C.; about −9° C.; about −8° C.; about −7° C.; about −6° C.; about −5° C.; about −4° C.; about-3° C.; about −2° C.; about −1° C.; about 0° C.; about 1° C.; about 2° C.; about 3° C.; about 4° C.; about 5° C.; about 6° C.; about 7° C.; about 8° C.; about 9° C.; about 10° C.; about 11° C.; about 12° C.; about 13° C.; about 14° C.; about 15° C.; about 16° C.; about 17° C.; about 18° C.; about 19° C.; about 20° C.; about 21° C.; about 22° C.; about 23° C.; about 24° C.; or about 25° C.; for 1 month or more.

In some embodiments, a pharmaceutical composition comprising, consisting essentially of, or consisting of a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Ac-Cys₁-Cth₂-Glu₃-Leu₄-Cys₅-Cys₆-Asn₇-Val₈-Ala₉-Cys₁₀-Tyr₁₁-Gly₁₂-Cys₁₃-OH or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; wherein Ac-indicates an acetylated N-terminus; and wherein-OH indicates an unmodified C-terminus, and an excipient; can be stable at about −20° C. for about 0.5 months; about 1 months; about 1.5 months; about 2 months; about 2.5 months; about 3 months; about 3.5 months; about 4 months; about 4.5 months; about 5 months; about 5.5 months; about 6 months; about 6.5 months; about 7 months; about 7.5 months; about 8 months; about 8.5 months; about 9 months; about 9.5 months; about 10 months; about 10.5 months; about 11 months; about 11.5 months; about 12 months; about 12.5 months; about 13 months; about 13.5 months; about 14 months; about 14.5 months; about 15 months; about 15.5 months; about 16 months; about 16.5 months; about 17 months; about 17.5 months; about 18 months; about 18.5 months; about 19 months; about 19.5 months; about 20 months; about 20.5 months; about 21 months; about 21.5 months; about 22 months; about 22.5 months; about 23 months; about 23.5 months; about 24 months; about 24.5 months; about 25 months; about 25.5 months; about 26 months; about 26.5 months; about 27 months; about 27.5 months; about 28 months; about 28.5 months; about 29 months; about 29.5 months; about 30 months; about 30.5 months; about 31 months; about 31.5 months; about 32 months; about 32.5 months; about 33 months; about 33.5 months; about 34 months; about 34.5 months; about 35 months; about 35.5 months; about 36 months; about 36.5 months; about 37 months; about 37.5 months; about 38 months; about 38.5 months; about 39 months; about 39.5 months; about 40 months; about 40.5 months; about 41 months; about 41.5 months; about 42 months; about 42.5 months; about 43 months; about 43.5 months; about 44 months; about 44.5 months; about 45 months; about 45.5 months; about 46 months; about 46.5 months; about 47 months; about 47.5 months; about 48 months; about 48.5 months; about 49 months; about 49.5 months; or about 50 months.

In some embodiments, a pharmaceutical composition comprising, consisting essentially of, or consisting of a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Ac-Cys₁-Cth₂-Glu₃-Leu₄-Cys₅-Cys₆-Asn₇-Val₈-Ala₉-Cys₁₀-Tyr₁₁-Gly₁₂-Cys₁₃-OH or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; wherein Ac-indicates an acetylated N-terminus; and wherein-OH indicates an unmodified C-terminus, and an excipient; can be stable at about 1.5° C., about 2° C., about 2.5° C., about 3° C., about 3.5° C., about 4° C., about 4.5° C., about 5° C., about 5.5° C., about 6° C., about 6.5° C., about 7° C., about 7.5° C., about 8° C., about 8.5° C., or about 9° C.; for about 0.5 months; about 1 months; about 1.5 months; about 2 months; about 2.5 months; about 3 months; about 3.5 months; about 4 months; about 4.5 months; about 5 months; about 5.5 months; about 6 months; about 6.5 months; about 7 months; about 7.5 months; about 8 months; about 8.5 months; or about 9 months.

In some embodiments, a pharmaceutical composition comprising, consisting essentially of, or consisting of a peptide having an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence to the amino acid sequence: Ac-Cys₁-Cth₂-Glu₃-Leu₄-Cys₅-Cys₆-Asn₇-Val₈-Ala₉-Cys₁₀-Tyr₁₁-Gly₁₂-Cys₁₃-OH or a pharmaceutically acceptable salt thereof; wherein Cth is a cystathionine; wherein Cys₁ and Cys₆; Cys₅ and Cys₁₃; are connected by disulfide bonds; and wherein Cth₂ and Cys₁₀ are connected by a thioether bond; wherein Ac-indicates an acetylated N-terminus; and wherein-OH indicates an unmodified C-terminus, and an excipient; can be stable at about 23° C., about 23.5° C., about 24° C., about 24.5° C., about 25° C., about 25.5° C., about 26° C., about 26.5° C., or about 27° C.; for about 0.1 months; about 0.2 months; about 0.3 months; about 0.4 months; about 0.5 months; about 0.6 months; about 0.7 months; about 0.8 months; about 0.9 months; about 1 months; about 1.1 months; about 1.2 months; about 1.3 months; about 1.4 months; or about 1.5 months.

In some embodiments, a pharmaceutical composition of the present disclosure can be stable at a temperature ranging from about −30° C. to about 30° C., or any temperature in between.

In some embodiments, a pharmaceutical composition of the present disclosure can be stable at a temperature ranging from about −20° C. to about 25° C., or any temperature in between.

In some embodiments, a pharmaceutical composition of the present disclosure can be stable at a temperature of about −20° C.

In some embodiments, a pharmaceutical composition of the present disclosure can be stable at a temperature ranging from about 2° C. to about 8° C.

In some embodiments, a pharmaceutical composition of the present disclosure can be stable at a temperature of about 5° C.

In some embodiments, a pharmaceutical composition of the present disclosure can be stable at a temperature of about 25° C.

In some embodiments, a pharmaceutical composition of the present disclosure can be stable at a temperature of about 25° C. with 60% relative humidity (RH).

In some embodiments, a pharmaceutical composition of the present disclosure can be stable at a temperature of about −20° C. for about 50 months, or more.

In some embodiments, a pharmaceutical composition of the present disclosure can be stable at a temperature of about −20° C. for about 0.5 months to about 48 months, or more, or any length of time in between.

In some embodiments, a pharmaceutical composition of the present disclosure can be stable at a temperature of about −20° C. for about 0.5 months to about 48 months, or more, or any length of time in between.

In some embodiments, a pharmaceutical composition of the present disclosure can be stable at a temperature ranging from about 2° C. to about 8° C., for about 12 months or more.

In some embodiments, a pharmaceutical composition of the present disclosure can be stable at a temperature ranging from about 2° C. to about 8° C., for about 6 months or more.

In some embodiments, a pharmaceutical composition of the present disclosure can be stable at a temperature ranging from about 2° C. to about 8° C., for about 6 months.

In some embodiments, a pharmaceutical composition of the present disclosure can be stable at a temperature ranging from about 2° C. to about 8° C., for about 0.5 months to about 12 months, or more, or any length of time in between.

In some embodiments, a pharmaceutical composition of the present disclosure can be stable at a temperature ranging from about 2° C. to about 8° C., for about 0.5 months to about 6 months, or more, or any length of time in between.

In some embodiments, a pharmaceutical composition of the present disclosure can be stable at a temperature ranging from about 2° C. to about 8° C., for about 0.5 months.

In some embodiments, a pharmaceutical composition of the present disclosure can be stable at a temperature ranging from about 2° C. to about 8° C., for about 3 months.

In some embodiments, a pharmaceutical composition of the present disclosure can be stable at a temperature ranging from about 2° C. to about 8° C., for about 6 months.

In some embodiments, a pharmaceutical composition of the present disclosure can be stable at a temperature of about 25° C. for about 1 month, or more.

In some embodiments, a pharmaceutical composition of the present disclosure can be stable at a temperature of about 25° C. for about 0.5 months to about 1 month, or any length of time in between.

In some embodiments, a pharmaceutical composition of the present disclosure can be stable at a temperature of about 25° C. for about 0.5 months.

In some embodiments, a pharmaceutical composition of the present disclosure can be stable at a temperature of about 25° C. for about 1 month.

In some embodiments, a pharmaceutical composition of the present disclosure can be stable at a temperature of about 25° C. with 60% relative humidity (RH) for about 1 month, or more.

In some embodiments, a pharmaceutical composition of the present disclosure can be stable at a temperature of about 25° C. with 60% RH for about 0.5 months to about 1 month, or any length of time in between.

In some embodiments, a pharmaceutical composition of the present disclosure can be stable at a temperature of about 25° C. with 60% RH for about 0.5 months.

In some embodiments, a pharmaceutical composition of the present disclosure can be stable at a temperature of about 25° C. with 60% RH for about 1 month.

### Assessing Stability of the Pharmaceutical Compositions

As used herein a “stable” pharmaceutical composition or formulation refers to a pharmaceutical composition or formulation in which the therapeutic or diagnostic agent retains an acceptable portion of its essential physical and/or chemical and/or biological properties over an acceptable period of time.

Exemplary methods of assessing stability of peptides are provided in Peptide and Protein Drug Delivery, 247-301, Vincent Lee Ed., Marcel Dekker, Inc., New York, N.Y., 1991; and Jones, A., Adv. Drug Delivery Rev. 10:29-90 (1993); the disclosures of which are incorporated herein by reference in their entireties.

In some embodiments, the chemical stability of a peptide, or a pharmaceutical composition thereof, can be assessed immediately after formulation, and/or again after a period of storage; and/or under different environmental conditions, e.g., temperature and humidity.

In some embodiments, the assays described herein can be used to determine the stability of a peptide of the present disclosure or a pharmaceutical composition thereof, for example, to determine the stability of the pharmaceutical compositions over time and/or at particular storage temperatures and conditions, by assessing activity, solubility, and stability (e.g. amount and/or activity of peptide; and/or presence of degradation products, etc.) prior to storage and then at various time points thereafter. The assays also can be used make minor adjustments to the formulations provided herein while retaining the stability of peptides.

In some embodiments, the stability of a pharmaceutical composition of the present disclosure can be assessed by visual assessment, acid clarification, optical microscopy, reversed phase high performance liquid chromatography (RP-HPLC), in vivo bioassays, and denaturing and non-denaturing size exclusion chromatography (SEC).

In some embodiments, stability can be determined by visual assessment, including changes in color, clarity, presence of aggregates or clumping and material adhesion, or frosting, to the vessel containing the pharmaceutical compositions provided herein. Visual changes can be confirmed by acid clarification, wherein lack of dissolution after acidification confirms the presence of insoluble denatured peptide. Visual changes can also be confirmed by optical microscopy and/or micrography by fluorescent backlighting.

In some embodiments, the stability of the pharmaceutical compositions of the present disclosure can be assessed based on activity of the peptides contained therein.

Activity of the peptides of the present disclosure can be assessed using methods and assays well known in the art. For example, the ability of a peptide of the present disclosure, including pharmaceutical compositions of the present disclosure comprising a peptide of the present disclosure, and their ability to act as a therapeutically effective agent, can be assessed in vitro or in vivo. For example, in vitro assays well known in the art can be performed to assess the ability of a peptide of the present disclosure to bind to one of its receptors.

For example, in some embodiments, the activity of a peptide of the present disclosure can be assessed based on the peptides ability to agonize guanylate cyclase C (GC-C), which can be assessed using techniques known to those having ordinary skill in the art.

In some embodiments, the activity of a peptide of the present disclosure can be assessed based on expression and/or activity downstream of the peptides pathway. For example, in some embodiments, the peptides ability stimulate and/or bind GC-C receptors; and/or the secretion of extracellular cyclic guanosine monophosphate (cGMP) across the basolateral membrane of colonic epithelial cells, can be evaluated to determine the activity of the peptides of the present disclosure.

In some embodiments, the stability of a pharmaceutical composition of the present disclosure can be determined by assessing degradation of the peptide of the present disclosure.

In some embodiments, the stability of a pharmaceutical composition of the present disclosure can be determined by assessing the identity of the contents thereof, e.g., via HPLC.

In some embodiments, the stability of a pharmaceutical composition of the present disclosure can be determined by assessing the purity of the pharmaceutical composition, e.g., via HPLC.

In some embodiments, the stability of a pharmaceutical composition of the present disclosure can be determined by assessing the quantity of related substances of the pharmaceutical composition, e.g., via HPLC.

In some embodiments, the stability of a pharmaceutical composition of the present disclosure can be determined by assessing the multimer content of the pharmaceutical composition, e.g., using size exclusion chromatography (SEC).

In some embodiments, the stability of a pharmaceutical composition of the present disclosure can be determined by using differential scanning calorimetry (DSC).

In some embodiments, the stability of a pharmaceutical composition of the present disclosure can be determined by assessing microbial load of the pharmaceutical composition, e.g., using the method described in USP <61>.

In some embodiments, the stability of a pharmaceutical composition of the present disclosure can be determined by assessing the water content of the pharmaceutical composition, e.g., via a Karl Fischer test.

In some embodiments, the stability of a pharmaceutical composition of the present disclosure can be determined by assessing the level of peptide degradation in the pharmaceutical composition.

Proteins, polypeptides, and peptides degrade in both biological samples and in solution (e.g., cell culture and/or during fermentation). Methods of detecting peptide degradation are well known in the art. Any of the well-known methods of detecting peptide degradation may be employed here.

In some embodiments, peptide degradation can be detected using isotope labeling techniques; liquid chromatography/mass spectrometry (LC/MS); HPLC; radioactive amino acid incorporation and subsequent detection, e.g., via scintillation counting; the use of a reporter protein, e.g., a protein that can be detected (e.g., by fluorescence, spectroscopy, luminometry, etc.); fluorescent intensity of one or more bioluminescent proteins and/or fluorescent proteins and/or fusions thereof; pulse-chase analysis (e.g., pulse-labeling a cell with radioactive amino acids and following the decay of the labeled protein while chasing with unlabeled precursor, and arresting protein synthesis and measuring the decay of total protein levels with time); or cycloheximide-chase assays.

In some embodiments, an assay can be used to detect peptide degradation, wherein a sample is contacted with a non-fluorescent compound that is operable to react with free primary amine in said sample produced via the degradation of a peptide, and which then produces a fluorescent signal that can be quantified and compared to a standard. Examples of non-fluorescent compounds that can be utilized as fluorescent tags for free amines according to the present disclosure are 3-(4-carboxybenzoyl) quinoline-2-carboxaldehyde (CBQCA), fluorescamine, and o-phthaldialdehyde.

In some embodiments, the method to determine the readout signal from the reporter protein depends from the nature of the reporter protein. For example, for fluorescent reporter proteins, the readout signal corresponds to the intensity of the fluorescent signal. The readout signal may be measured using spectroscopy-, fluorometry-, photometry-, and/or luminometry-based methods and detection systems, for example. Such methods and detection systems are well known in the art.

In some embodiments, standard immunological procedures known to those having ordinary skill in the art can be used to detect peptide degradation. For example, in some embodiments, peptide degradation can be detected in a sample using immunoassays that employ a detectable antibody. Such immunoassays include, for example, agglutination assays, ELISA, Pandex microfluorimetric assay, flow cytometry, serum diagnostic assays, and immunohistochemical staining procedures, all of which are well-known in the art. In some embodiments, the levels (e.g., of fluorescence) in one sample can be compared to a standard. An antibody can be made detectable by various means well known in the art. For example, a detectable marker can be directly or indirectly attached to the antibody. Useful markers include, for example, radionucleotides, enzymes, fluorogens, chromogens and chemiluminescent labels.

Exemplary methods of detecting peptide degradation is provided in U.S. Pat. Nos. 5,766,927; 7,504,253; 9,201,073; 9,429,566; U.S. patent application Ser. No. 20/120,028286; Eldeeb et al., A molecular toolbox for studying protein degradation in mammalian cells. J Neurochem. 2019 November; 151 (4): 520-533; and Buchanan et al., Cycloheximide Chase Analysis of Protein Degradation in Saccharomyces cerevisiae. J Vis Exp. 2016; (110): 53975, the disclosures of which are incorporated herein by reference in their entireties.

### Methods of Use and Administration

The present disclosure provides peptides and/or mixtures, combinations, compositions, and/or formulations comprising the same, for use in a method for the treatment and/or prevention of certain visceral pain conditions of the abdominal region, e.g., including without limitation, bladder pain associated with interstitial cystitis/bladder pain syndrome (IC/BPS), and endometriosis pain, and administering to a patient in need thereof, a therapeutically effective amount of a peptides of the present disclosure, or pharmaceutical compositions thereof.

In some embodiments, the present disclosure provides a method comprising, consisting essentially of, or consisting of, administering to the subject a therapeutically effective amount of one or more peptides described herein, or a pharmaceutically acceptable salt, hydrate or solvate thereof, which is optionally in a composition. In certain embodiments, the method further comprises administering to the subject an additional therapeutic agent that treats or prevents abdominal pain.

In some embodiments, the present disclosure provides a method comprising, consisting essentially of, or consisting of, administering to the subject a therapeutically effective amount of pharmaceutical composition comprising one or more peptides described herein, or a pharmaceutically acceptable salt, hydrate or solvate thereof. In certain embodiments, the method further comprises administering to the subject an additional therapeutic agent that treats or prevents abdominal pain.

In some embodiments, the present disclosure provides a method comprising, consisting essentially of, or consisting of, contacting a subject or a cell therefrom with an effective amount of a peptide of the present disclosure, a pharmaceutically acceptable salt, hydrate or solvate thereof, or a pharmaceutical composition thereof, as defined herein.

In some embodiments, the present disclosure provides a method comprising, consisting essentially of, or consisting of, treating a disease or disorder in which visceral pain conditions of the abdominal region, e.g., including without limitation, bladder pain associated with interstitial cystitis/bladder pain syndrome (IC/BPS), is implicated in a subject in need of such treatment, said method comprising, consisting essentially of, or consisting of, administering to said patient a therapeutically effective amount of a peptide of the present disclosure, or a pharmaceutically acceptable salt, hydrate or solvate thereof, or a pharmaceutical composition as defined herein.

In some embodiments, the present disclosure provides a method comprising, consisting essentially of, or consisting of, treating bladder pain associated with interstitial cystitis/bladder pain syndrome (IC/BPS), in a subject in need of such treatment, said method comprising, consisting essentially of, or consisting of, administering to said patient a therapeutically effective amount of a peptide of the present disclosure, or a pharmaceutically acceptable salt, hydrate or solvate thereof, or a pharmaceutical composition as defined herein.

In some embodiments, the present disclosure comprises, consists essentially of, or consists of, the use of one or more of the peptides and/or pharmaceutical compositions described herein for therapeutic purposes.

In some embodiments, the present disclosure comprises, consists essentially of, or consists of, the use of one or more of the peptides and/or pharmaceutical compositions described herein for prophylactic purposes.

Any of the peptides of the present disclosure, and/or any of the pharmaceutical compositions, liquid pharmaceutical compositions, unit dosage form, or pharmaceutical rectal foam compositions of the present disclosure, can be used to practice the methods of the present disclosure. For example, a peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃ are connected by disulfide bonds; and wherein Cth₂ and Cys₁₀ are connected by a thioether bond; and wherein the peptide has an N-terminus that is optionally acetylated, or a pharmaceutically acceptable salt, hydrate or solvate thereof; or a pharmaceutical composition, liquid pharmaceutical composition, unit dosage form, or pharmaceutical rectal foam composition comprising the same, can be used in the method contemplated by the present disclosure to treat a visceral pain condition in a subject in need thereof, wherein the method comprises: selecting a patient having a visceral pain condition, and administering to the subject a therapeutically effective amount of the peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃ are connected by disulfide bonds; and wherein Cth₂ and Cys₁₀ are connected by a thioether bond; wherein the peptide has an N-terminus that is optionally acetylated, or a pharmaceutically acceptable salt, hydrate or solvate thereof; or a pharmaceutical composition, liquid pharmaceutical composition, unit dosage form, or pharmaceutical rectal foam composition comprising the same.

### Method of Treating a Visceral Pain Condition

In some embodiments, the present disclosure provides a method of treating, limiting the symptoms of, reducing the severity of, or relieving the pain associated with, a visceral pain condition, in a subject in need thereof.

In some embodiments, the present disclosure provides a method of treating, limiting the symptoms of, reducing the severity of, or relieving the pain associated with, a urogenital disorder, in a subject in need thereof.

In some embodiments, the present disclosure provides a method of treating, limiting the symptoms of, reducing the severity of, or relieving the pain associated with, a genitourinary disorder, in a subject in need thereof.

In some embodiments, the present disclosure provides a method of treating, limiting the symptoms of, reducing the severity of, or relieving the pain associated with, a bladder disorder, in a subject in need thereof.

In some embodiments, the present disclosure provides a method of treating, limiting the symptoms of, reducing the severity of, or relieving the pain associated with, a visceral pain condition, in a subject in need thereof; wherein the visceral pain condition is selected from: bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology; diverticulitis pain; pain associated with gastrointestinal disorders; pain associated with venereal diseases; pain associated with irritable bowel syndrome (IBS); rectal pain; chronic proctalgia; proctalgia fugax; anal pain; chronic anal fissure; post-operative anal pain; pain associated with cancer; pain associated with gastrointestinal tract neoplasms; general pelvic pain; orchialgia; chronic prostatitis; prostatodynia; vulvodynia; urethral syndrome; penile pain; perianal pain; and pain associated with ulcerative colitis; ulcerative proctitis; Crohn's disease; or any combination thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of a peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

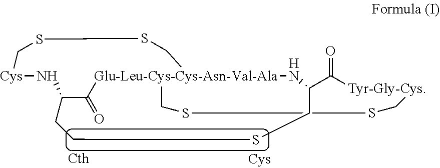

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; or a pharmaceutically acceptable salt, hydrate or solvate thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of a peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

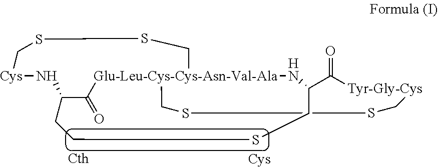

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; or a pharmaceutically acceptable salt, hydrate or solvate thereof; wherein administering the therapeutically effective amount of the peptide, or the pharmaceutically acceptable salt, hydrate or solvate thereof, limits the symptoms of, reduces the severity of, or treats the visceral pain condition in the subject in need thereof, relative to the symptoms and/or severity thereof in the subject prior to receiving the therapeutically effective amount of the peptide, or the pharmaceutically acceptable salt, hydrate or solvate thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of a peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

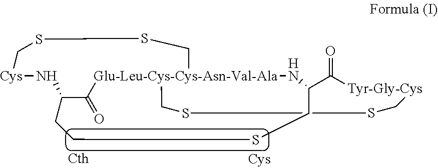

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; or a pharmaceutically acceptable salt, hydrate or solvate thereof; wherein the subject in need thereof who is administered the therapeutically effective amount of the peptide, or the pharmaceutically acceptable salt, hydrate or solvate thereof, has a reduction in the number and/or severity of symptoms of the visceral pain condition, as compared to a subject who is not administered the therapeutically effective amount of the peptide, or the pharmaceutically acceptable salt, hydrate or solvate thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, the present disclosure provides a method of limiting the symptoms of, reducing the severity of, or treating a visceral pain condition, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, the present disclosure provides a method of limiting the symptoms of, reducing the severity of, or treating a visceral pain condition, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein administering the therapeutically effective amount of the peptide, or the pharmaceutically acceptable salt, hydrate or solvate thereof, limits the number of symptoms of the visceral pain condition, reduces the severity of symptoms of the visceral pain condition, reduces the level of pain associated with the visceral pain condition, and/or treats the visceral pain condition in the subject in need thereof; relative to the number of symptoms, severity of symptoms, or level of pain associated with the visceral pain condition experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein the subject in need thereof who is administered the therapeutically effective amount of the peptide, or the pharmaceutically acceptable salt, hydrate or solvate thereof, has a reduction in the number and/or severity of symptoms of the visceral pain condition, as compared to a subject who is not administered the therapeutically effective amount of the peptide, or the pharmaceutically acceptable salt, hydrate or solvate thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein the subject in need thereof who is administered the therapeutically effective amount of the peptide, or the pharmaceutically acceptable salt, hydrate or solvate thereof, has a reduction in the number and/or severity of symptoms of the visceral pain condition, as compared to the number and/or severity of symptoms of the visceral pain condition experience by the subject prior to being administered the therapeutically effective amount of the peptide, or the pharmaceutically acceptable salt, hydrate or solvate thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of (1) a peptide of the present disclosure, or a pharmaceutically acceptable salt, hydrate, or solvate thereof; or (2) a pharmaceutical composition of the present disclosure (e.g., a pharmaceutical rectal foam composition); wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, the present disclosure provides a method of limiting the symptoms of, reducing the severity of, or treating a visceral pain condition, in a subject in need thereof, the method comprising: administering to the subject in need thereof a therapeutically effective amount of (1) a peptide of the present disclosure, or a pharmaceutically acceptable salt, hydrate, or solvate thereof; or (2) a pharmaceutical composition of the present disclosure (e.g., a pharmaceutical rectal foam composition); wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein administering the therapeutically effective amount of the peptide, or the pharmaceutically acceptable salt, hydrate or solvate thereof, limits the number of symptoms of the visceral pain condition, reduces the severity of symptoms of the visceral pain condition, reduces the level of pain associated with the visceral pain condition, and/or treats the visceral pain condition in the subject in need thereof; relative to the number of symptoms, severity of symptoms, or level of pain associated with the visceral pain condition experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of (1) a peptide of the present disclosure, or a pharmaceutically acceptable salt, hydrate, or solvate thereof; or (2) a pharmaceutical composition of the present disclosure (e.g., a pharmaceutical rectal foam composition); wherein administering the therapeutically effective amount of the peptide, or the pharmaceutically acceptable salt, hydrate or solvate thereof, limits the number of symptoms of the visceral pain condition, reduces the severity of symptoms of the visceral pain condition, reduces the level of pain associated with the visceral pain condition, and/or treats the visceral pain condition in the subject in need thereof; relative to the number of symptoms, severity of symptoms, or level of pain associated with the visceral pain condition experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating a visceral pain condition, in a subject in need thereof, the method comprising: administering to the subject in need thereof a therapeutically effective amount of (1) a peptide of the present disclosure, or a pharmaceutically acceptable salt, hydrate, or solvate thereof; or (2) a pharmaceutical composition of the present disclosure (e.g., a pharmaceutical rectal foam composition); wherein the visceral pain condition is selected from: bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology; diverticulitis pain; pain associated with gastrointestinal disorders; pain associated with venereal diseases; pain associated with irritable bowel syndrome (IBS); rectal pain; chronic proctalgia; proctalgia fugax; anal pain; chronic anal fissure; post-operative anal pain; pain associated with cancer; pain associated with gastrointestinal tract neoplasms; general pelvic pain; orchialgia; chronic prostatitis; prostatodynia; vulvodynia; urethral syndrome; penile pain; perianal pain; and pain associated with ulcerative colitis; ulcerative proctitis; Crohn's disease; or any combination thereof.

Any of the peptides of the present disclosure, and/or any of the pharmaceutical compositions, liquid pharmaceutical compositions, unit dosage form, or pharmaceutical rectal foam compositions of the present disclosure, can be used to practice the methods of the present disclosure. For example, a peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃; are connected by disulfide bonds; and wherein Cth₂ and Cys₁₀ are connected by a thioether bond; or a pharmaceutically acceptable salt, hydrate or solvate thereof, or a pharmaceutical composition, liquid pharmaceutical composition, unit dosage form, or pharmaceutical rectal foam composition comprising the same, can be used in the method contemplated by the present disclosure to treat a visceral pain condition in a subject in need thereof, wherein the method comprises: selecting a patient having a visceral pain condition, and administering to the subject a therapeutically effective amount of the peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃; are connected by disulfide bonds; and wherein Cth₂ and Cys₁₀ are connected by a thioether bond; or a pharmaceutically acceptable salt, hydrate or solvate thereof, or a pharmaceutical composition, liquid pharmaceutical composition, unit dosage form, or pharmaceutical rectal foam composition comprising the same.

### Method of Treating IC/BPS

In some embodiments, the present disclosure provides a method of treating, limiting the symptoms of, reducing the severity of, or relieving the pain associated with, interstitial cystitis/bladder pain syndrome (IC/BPS), in a subject in need thereof.

In some embodiments, the present disclosure provides a method of treating, limiting the symptoms of, reducing the severity of, or relieving the pain associated with, interstitial cystitis/bladder pain syndrome (IC/BPS), in a subject in need thereof; wherein the pain associated with IC/BPS is selected from: urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; or any combination thereof.

In some embodiments, the present disclosure provides a method of limiting the symptoms of, reducing the severity of, or treating interstitial cystitis/bladder pain syndrome (IC/BPS), in a subject in need thereof, the method comprising: administering to the subject in need thereof a therapeutically effective amount of a peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

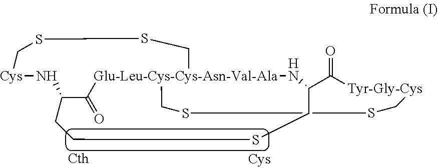

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; or a pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of a peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

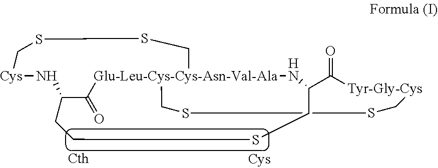

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; or a pharmaceutically acceptable salt, hydrate or solvate thereof; wherein administering the therapeutically effective amount of the peptide, or the pharmaceutically acceptable salt, hydrate or solvate thereof, limits the number of symptoms of IC/BPS, reduces the severity of symptoms of IC/BPS, reduces the level of pain associated with IC/BPS, and/or treats IC/BPS in the subject in need thereof; relative to the number of symptoms of, severity of symptoms of, or level of pain associated with IC/BPS, experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of a peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

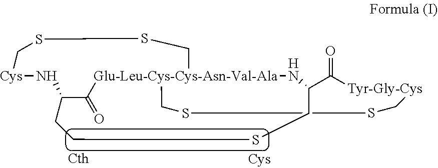

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; or a pharmaceutically acceptable salt, hydrate or solvate thereof; wherein the subject in need thereof who is administered the therapeutically effective amount of the peptide, or the pharmaceutically acceptable salt, hydrate or solvate thereof, has a reduction in the number and/or severity of symptoms of IC/BPS, as compared to a subject who is not administered the therapeutically effective amount of the peptide, or the pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of a peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

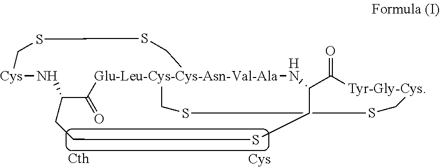

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; or a pharmaceutically acceptable salt, hydrate or solvate thereof; wherein administering the therapeutically effective amount of the peptide, or the pharmaceutically acceptable salt, hydrate or solvate thereof, limits the symptoms of, reduces the severity of, or treats the IC/BPS in the subject in need thereof, relative to the symptoms experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of a peptide comprising an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

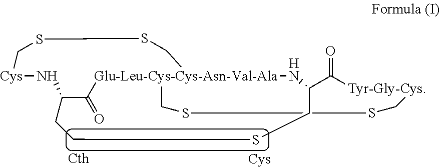

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; or a pharmaceutically acceptable salt, hydrate or solvate thereof; wherein the subject in need thereof who is administered the therapeutically effective amount of the peptide, or the pharmaceutically acceptable salt, hydrate or solvate thereof, has a reduction in the number and/or severity of symptoms of IC/BPS, as compared to the symptoms experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, the present disclosure provides a method of limiting the symptoms of, reducing the severity of, or treating IC/BPS, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, the present disclosure provides a method of limiting the symptoms of, reducing the severity of, or treating IC/BPS, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein administering the therapeutically effective amount of the peptide, or the pharmaceutically acceptable salt, hydrate or solvate thereof, limits the number of symptoms of IC/BPS, reduces the severity of symptoms of IC/BPS, reduces the level of pain associated with IC/BPS, and/or treats IC/BPS in the subject in need thereof; relative to the number of symptoms, severity of symptoms, or level of pain associated with IC/BPS experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein the subject in need thereof who is administered the therapeutically effective amount of the peptide, or the pharmaceutically acceptable salt, hydrate or solvate thereof, has a reduction in the number and/or severity of symptoms of IC/BPS, as compared to a subject who is not administered the therapeutically effective amount of the peptide, or the pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of (1) a peptide of the present disclosure, or a pharmaceutically acceptable salt, hydrate, or solvate thereof; or (2) a pharmaceutical composition of the present disclosure (e.g., a pharmaceutical rectal foam composition); wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, the present disclosure provides a method of limiting the symptoms of, reducing the severity of, or treating IC/BPS, in a subject in need thereof, the method comprising: administering to the subject in need thereof a therapeutically effective amount of (1) a peptide of the present disclosure, or a pharmaceutically acceptable salt, hydrate, or solvate thereof; or (2) a pharmaceutical composition of the present disclosure (e.g., a pharmaceutical rectal foam composition); wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein administering the therapeutically effective amount of the peptide, or the pharmaceutically acceptable salt, hydrate or solvate thereof, limits the number of symptoms of IC/BPS, reduces the severity of symptoms of IC/BPS, reduces the level of pain associated with IC/BPS, and/or treats IC/BPS in the subject in need thereof; relative to the number of symptoms, severity of symptoms, or level of pain associated with IC/BPS experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of (1) a peptide of the present disclosure, or a pharmaceutically acceptable salt, hydrate, or solvate thereof; or (2) a pharmaceutical composition of the present disclosure (e.g., a pharmaceutical rectal foam composition); wherein administering the therapeutically effective amount of the peptide, or the pharmaceutically acceptable salt, hydrate or solvate thereof, limits the number of symptoms of IC/BPS, reduces the severity of symptoms of IC/BPS, reduces the level of pain associated with IC/BPS, and/or treats IC/BPS in the subject in need thereof; relative to the number of symptoms of, severity of symptoms of, or level of pain associated with IC/BPS, experienced by the subject prior to the administration of the therapeutically effective amount of the peptide, or the pharmaceutically acceptable salt, hydrate or solvate thereof.

In some embodiments, the present disclosure provides a method of limiting the symptoms of, reducing the severity of, or treating IC/BPS, in a subject in need thereof, the method comprising: administering to the subject in need thereof a therapeutically effective amount of (1) a peptide of the present disclosure, or a pharmaceutically acceptable salt, hydrate, or solvate thereof; or (2) a pharmaceutical composition of the present disclosure (e.g., a pharmaceutical rectal foam composition); wherein the symptom of IC/BPS is selected from: bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; or any combination thereof.

Any of the peptides of the present disclosure, and/or any of the pharmaceutical compositions, liquid pharmaceutical compositions, unit dosage form, or pharmaceutical rectal foam compositions of the present disclosure, can be used to practice the methods of the present disclosure, i.e., a method of treating IC/BPS in a subject in need thereof. For example, a peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃ are connected by disulfide bonds; and wherein Cth₂ and Cys₁₀ are connected by a thioether bond; and wherein the peptide has an N-terminus that is optionally acetylated, or a pharmaceutically acceptable salt, hydrate or solvate thereof; or a pharmaceutical composition, liquid pharmaceutical composition, unit dosage form, or pharmaceutical rectal foam composition comprising the same, can be used in the method contemplated by the present disclosure to treat a visceral pain condition in a subject in need thereof, wherein the method comprises: selecting a patient having IC/BPs, and administering to the subject a therapeutically effective amount of the peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃ are connected by disulfide bonds; and wherein Cth₂ and Cys₁₀ are connected by a thioether bond; wherein the peptide has an N-terminus that is optionally acetylated, or a pharmaceutically acceptable salt, hydrate or solvate thereof; or a pharmaceutical composition, liquid pharmaceutical composition, unit dosage form, or pharmaceutical rectal foam composition comprising the same.

### Dosing

The terms “dose,” and “dosage” and the like refer, in the usual and customary sense, to the amount of active ingredient given to an individual at each administration. For the methods, compounds, and compositions provided herein, the dose may generally refer to the amount of treatment used to treat a disease. In some embodiments, the dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress.

The term “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Peptides of the present disclosure, and pharmaceutical compositions thereof, can be packaged in single or multiple unit dosage form for ease of administration and uniformity of dosage. The unit dosage form may be for a single daily dose or one of multiple daily doses (e.g., about 1 to 4 or more times per day). When multiple daily doses are used, the unit dosage form may be the same or different for each dose.

In some embodiments, the unit dosage form, and the particular format thereof, i.e., the particular formulation of the pharmaceutical composition, depends on the route of administration, e.g., any route of administration as described herein.

In some embodiments, a pharmaceutical composition can contain a plurality of active ingredients (e.g., two active ingredients) in a plurality of separate unit dosage forms (e.g., a separate dosage form for each of two active ingredients). In other embodiments, the pharmaceutical composition can contain a single unit dosage form (e.g., a single pill, tablet injection aliquot or the like) wherein the single unit dosage form includes a plurality of active ingredients (e.g., two active ingredients).

In some embodiments, the unit dosage form of the pharmaceutical composition is subdivided into unit doses containing appropriate quantities of the active components. In some embodiments, such unit dosage forms of the pharmaceutical composition is subdivided into unit doses containing appropriate quantities of the active components, each component contained within a separate unit dosage form. The unit dosage form can be a packaged preparation, the package containing discrete quantities of the pharmaceutical composition or separate active ingredients of the pharmaceutical composition, such as packeted tablets, capsules, and powders in vials or ampoules. Also, the unit dosage form can be a capsule, tablet, cachet, or lozenge itself, or it can be the appropriate number of any of these in packaged form.

The therapeutically effective amount or dose of a peptide of the present disclosure depends on the age, sex and weight of the patient, the current medical condition of the patient and the progression of disease in the patient being treated. The skilled artisan is able to determine appropriate dosages depending on these and other factors.

In some embodiments, a peptide of the present disclosure as described herein, or a pharmaceutically acceptable salt, hydrate or solvate thereof, or a pharmaceutical composition comprising, consisting essentially of, or consisting of a peptide of the present disclosure, can administered to a subject in need thereof, at a dose of from about 50 μg to about 5000 μg, and every sub-range within the foregoing cited ranges and amounts.

In some embodiments, one or more peptides described herein, or a pharmaceutically acceptable salt, hydrate or solvate thereof, or a pharmaceutical composition comprising, consisting essentially of, or consisting of a peptide of the present disclosure, can administered to a subject in need thereof, at a dose of from about 100 μg to about 2500 μg, and every sub-range within the foregoing cited ranges and amounts.

In yet other embodiments, a dose of the peptide of the present disclosure to be administered to a subject in need thereof can include a single dose of about 50 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 510 μg, about 520 μg, about 530 μg, about 540 μg, about 550 μg, about 560 μg, about 570 μg, about 580 μg, about 590 μg, about 600 μg, about 610 μg, about 620 μg, about 630 μg, about 640 μg, about 650 μg, about 660 μg, about 670 μg, about 680 μg, about 690 μg, about 700 μg, about 710 μg, about 720 μg, about 730 μg, about 740 μg, about 750 μg, about 760 μg, about 770 μg, about 780 μg, about 790 μg, about 800 μg, about 810 μg, about 820 μg, about 830 μg, about 840 μg, about 850 μg, about 860 μg, about 870 μg, about 880 μg, about 890 μg, about 900 μg, about 910 μg, about 920 μg, about 930 μg, about 940 μg, about 950 μg, about 960 μg, about 970 μg, about 980 μg, about 990 μg, about 1000 μg, about 1010 μg, about 1020 μg, about 1030 μg, about 1040 μg, about 1050 μg, about 1060 μg, about 1070 μg, about 1080 μg, about 1090 μg, about 1100 μg, about 1110 μg, about 1120 μg, about 1130 μg, about 1140 μg, about 1150 μg, about 1160 μg, about 1170 μg, about 1180 μg, about 1190 μg, about 1200 μg, about 1210 μg, about 1220 μg, about 1230 μg, about 1240 μg, about 1250 μg, about 1260 μg, about 1270 μg, about 1280 μg, about 1290 μg, about 1300 μg, about 1310 μg, about 1320 μg, about 1330 μg, about 1340 μg, about 1350 μg, about 1360 μg, about 1370 μg, about 1380 μg, about 1390 μg, about 1400 μg, about 1410 μg, about 1420 μg, about 1430 μg, about 1440 μg, about 1450 μg, about 1460 μg, about 1470 μg, about 1480 μg, about 1490 μg, about 1500 μg, about 1510 μg, about 1520 μg, about 1530 μg, about 1540 μg, about 1550 μg, about 1560 μg, about 1570 μg, about 1580 μg, about 1590 μg, about 1600 μg, about 1610 μg, about 1620 μg, about 1630 μg, about 1640 μg, about 1650 μg, about 1660 μg, about 1670 μg, about 1680 μg, about 1690 μg, about 1700 μg, about 1710 μg, about 1720 μg, about 1730 μg, about 1740 μg, about 1750 μg, about 1760 μg, about 1770 μg, about 1780 μg, about 1790 μg, about 1800 μg, about 1810 μg, about 1820 μg, about 1830 μg, about 1840 μg, about 1850 μg, about 1860 μg, about 1870 μg, about 1880 μg, about 1890 μg, about 1900 μg, about 1910 μg, about 1920 μg, about 1930 μg, about 1940 μg, about 1950 μg, about 1960 μg, about 1970 μg, about 1980 μg, about 1990 μg, about 2000 μg, about 2010 μg, about 2020 μg, about 2030 μg, about 2040 μg, about 2050 μg, about 2060 μg, about 2070 μg, about 2080 μg, about 2090 μg, about 2100 μg, about 2110 μg, about 2120 μg, about 2130 μg, about 2140 μg, about 2150 μg, about 2160 μg, about 2170 μg, about 2180 μg, about 2190 μg, about 2200 μg, about 2210 μg, about 2220 μg, about 2230 μg, about 2240 μg, about 2250 μg, about 2260 μg, about 2270 μg, about 2280 μg, about 2290 μg, about 2300 μg, about 2310 μg, about 2320 μg, about 2330 μg, about 2340 μg, about 2350 μg, about 2360 μg, about 2370 μg, about 2380 μg, about 2390 μg, about 2400 μg, about 2410 μg, about 2420 μg, about 2430 μg, about 2440 μg, about 2450 μg, about 2460 μg, about 2470 μg, about 2480 μg, about 2490 μg, about 2500 μg, or about 3000 μg.

By way of illustration only, and taking into consideration various factors for determining appropriate doses and dosing frequencies, a dose of the peptide of the present disclosure to be administered to a subject in need thereof can include a single dose of about 100 μg, about 300 μg, about 600 μg, about 900 μg, about 1800 μg, or about 2500 μg.

In some embodiments, a dose of the peptide of the present disclosure to be administered to a subject in need thereof can include a single dose of about 50 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 510 μg, about 520 μg, about 530 μg, about 540 μg, about 550 μg, about 560 μg, about 570 μg, about 580 μg, about 590 μg, about 600 μg, about 610 μg, about 620 μg, about 630 μg, about 640 μg, about 650 μg, about 660 μg, about 670 μg, about 680 μg, about 690 μg, about 700 μg, about 710 μg, about 720 μg, about 730 μg, about 740 μg, about 750 μg, about 760 μg, about 770 μg, about 780 μg, about 790 μg, about 800 μg, about 810 μg, about 820 μg, about 830 μg, about 840 μg, about 850 μg, about 860 μg, about 870 μg, about 880 μg, about 890 μg, about 900 μg, about 910 μg, about 920 μg, about 930 μg, about 940 μg, about 950 μg, about 960 μg, about 970 μg, about 980 μg, about 990 μg, about 1000 μg, about 1010 μg, about 1020 μg, about 1030 μg, about 1040 μg, about 1050 μg, about 1060 μg, about 1070 μg, about 1080 μg, about 1090 μg, about 1100 μg, about 1110 μg, about 1120 μg, about 1130 μg, about 1140 μg, about 1150 μg, about 1160 μg, about 1170 μg, about 1180 μg, about 1190 μg, about 1200 μg, about 1210 μg, about 1220 μg, about 1230 μg, about 1240 μg, about 1250 μg, about 1260 μg, about 1270 μg, about 1280 μg, about 1290 μg, about 1300 μg, about 1310 μg, about 1320 μg, about 1330 μg, about 1340 μg, about 1350 μg, about 1360 μg, about 1370 μg, about 1380 μg, about 1390 μg, about 1400 μg, about 1410 μg, about 1420 μg, about 1430 μg, about 1440 μg, about 1450 μg, about 1460 μg, about 1470 μg, about 1480 μg, about 1490 μg, about 1500 μg, about 1510 μg, about 1520 μg, about 1530 μg, about 1540 μg, about 1550 μg, about 1560 μg, about 1570 μg, about 1580 μg, about 1590 μg, about 1600 μg, about 1610 μg, about 1620 μg, about 1630 μg, about 1640 μg, about 1650 μg, about 1660 μg, about 1670 μg, about 1680 μg, about 1690 μg, about 1700 μg, about 1710 μg, about 1720 μg, about 1730 μg, about 1740 μg, about 1750 μg, about 1760 μg, about 1770 μg, about 1780 μg, about 1790 μg, about 1800 μg, about 1810 μg, about 1820 μg, about 1830 μg, about 1840 μg, about 1850 μg, about 1860 μg, about 1870 μg, about 1880 μg, about 1890 μg, about 1900 μg, about 1910 μg, about 1920 μg, about 1930 μg, about 1940 μg, about 1950 μg, about 1960 μg, about 1970 μg, about 1980 μg, about 1990 μg, about 2000 μg, about 2010 μg, about 2020 μg, about 2030 μg, about 2040 μg, about 2050 μg, about 2060 μg, about 2070 μg, about 2080 μg, about 2090 μg, about 2100 μg, about 2110 μg, about 2120 μg, about 2130 μg, about 2140 μg, about 2150 μg, about 2160 μg, about 2170 μg, about 2180 μg, about 2190 μg, about 2200 μg, about 2210 μg, about 2220 μg, about 2230 μg, about 2240 μg, about 2250 μg, about 2260 μg, about 2270 μg, about 2280 μg, about 2290 μg, about 2300 μg, about 2310 μg, about 2320 μg, about 2330 μg, about 2340 μg, about 2350 μg, about 2360 μg, about 2370 μg, about 2380 μg, about 2390 μg, about 2400 μg, about 2410 μg, about 2420 μg, about 2430 μg, about 2440 μg, about 2450 μg, about 2460 μg, about 2470 μg, about 2480 μg, about 2490 μg, about 2500 μg, or about 3000 μg, administered once or more times per day, and/or one or more times per week, for example, for one to four weeks, or one to eight weeks, or one to twelve weeks, or one to fourteen weeks.

In some embodiments, a dose of the peptide of the present disclosure to be administered to a subject in need thereof can include a single dose of about 50 μg, about 100 μg, about 300 μg, about 600 μg, about 900 μg, about 1800 μg, about 2500 μg or about 3000 μg, administered once or more times per day, and/or one or more times per week, for example, for one to four weeks, or one to eight weeks, or one to twelve weeks, or one to fourteen weeks.

In some embodiments, a dosing regimen can include administration of a maximal dose or dosing frequency that avoids significant undesirable side effects. In some embodiments, a total daily dose of the peptide of the present disclosure may be at least about 50 μg, at least about 100 μg, at least about 110 μg, at least about 120 μg, at least about 130 μg, at least about 140 μg, at least about 150 μg, at least about 160 μg, at least about 170 μg, at least about 180 μg, at least about 190 μg, at least about 200 μg, at least about 210 μg, at least about 220 μg, at least about 230 μg, at least about 240 μg, at least about 250 μg, at least about 260 μg, at least about 270 μg, at least about 280 μg, at least about 290 μg, at least about 300 μg, at least about 310 μg, at least about 320 μg, at least about 330 μg, at least about 340 μg, at least about 350 μg, at least about 360 μg, at least about 370 μg, at least about 380 μg, at least about 390 μg, at least about 400 μg, at least about 410 μg, at least about 420 μg, at least about 430 μg, at least about 440 μg, at least about 450 μg, at least about 460 μg, at least about 470 μg, at least about 480 μg, at least about 490 μg, at least about 500 μg, at least about 510 μg, at least about 520 μg, at least about 530 μg, at least about 540 μg, at least about 550 μg, at least about 560 μg, at least about 570 μg, at least about 580 μg, at least about 590 μg, at least about 600 μg, at least about 610 μg, at least about 620 μg, at least about 630 μg, at least about 640 μg, at least about 650 μg, at least about 660 μg, at least about 670 μg, at least about 680 μg, at least about 690 μg, at least about 700 μg, at least about 710 μg, at least about 720 μg, at least about 730 μg, at least about 740 μg, at least about 750 μg, at least about 760 μg, at least about 770 μg, at least about 780 μg, at least about 790 μg, at least about 800 μg, at least about 810 μg, at least about 820 μg, at least about 830 μg, at least about 840 μg, at least about 850 μg, at least about 860 μg, at least about 870 μg, at least about 880 μg, at least about 890 μg, at least about 900 μg, at least about 910 μg, at least about 920 μg, at least about 930 μg, at least about 940 μg, at least about 950 μg, at least about 960 μg, at least about 970 μg, at least about 980 μg, at least about 990 μg, at least about 1000 μg, at least about 1010 μg, at least about 1020 μg, at least about 1030 μg, at least about 1040 μg, at least about 1050 μg, at least about 1060 μg, at least about 1070 μg, at least about 1080 μg, at least about 1090 μg, at least about 1100 μg, at least about 1110 μg, at least about 1120 μg, at least about 1130 μg, at least about 1140 μg, at least about 1150 μg, at least about 1160 μg, at least about 1170 μg, at least about 1180 μg, at least about 1190 μg, at least about 1200 μg, at least about 1210 μg, at least about 1220 μg, at least about 1230 μg, at least about 1240 μg, at least about 1250 μg, at least about 1260 μg, at least about 1270 μg, at least about 1280 μg, at least about 1290 μg, at least about 1300 μg, at least about 1310 μg, at least about 1320 μg, at least about 1330 μg, at least about 1340 μg, at least about 1350 μg, at least about 1360 μg, at least about 1370 μg, at least about 1380 μg, at least about 1390 μg, at least about 1400 μg, at least about 1410 μg, at least about 1420 μg, at least about 1430 μg, at least about 1440 μg, at least about 1450 μg, at least about 1460 μg, at least about 1470 μg, at least about 1480 μg, at least about 1490 μg, at least about 1500 μg, at least about 1510 μg, at least about 1520 μg, at least about 1530 μg, at least about 1540 μg, at least about 1550 μg, at least about 1560 μg, at least about 1570 μg, at least about 1580 μg, at least about 1590 μg, at least about 1600 μg, at least about 1610 μg, at least about 1620 μg, at least about 1630 μg, at least about 1640 μg, at least about 1650 μg, at least about 1660 μg, at least about 1670 μg, at least about 1680 μg, at least about 1690 μg, at least about 1700 μg, at least about 1710 μg, at least about 1720 μg, at least about 1730 μg, at least about 1740 μg, at least about 1750 μg, at least about 1760 μg, at least about 1770 μg, at least about 1780 μg, at least about 1790 μg, at least about 1800 μg, at least about 1810 μg, at least about 1820 μg, at least about 1830 μg, at least about 1840 μg, at least about 1850 μg, at least about 1860 μg, at least about 1870 μg, at least about 1880 μg, at least about 1890 μg, at least about 1900 μg, at least about 1910 μg, at least about 1920 μg, at least about 1930 μg, at least about 1940 μg, at least about 1950 μg, at least about 1960 μg, at least about 1970 μg, at least about 1980 μg, at least about 1990 μg, at least about 2000 μg, at least about 2010 μg, at least about 2020 μg, at least about 2030 μg, at least about 2040 μg, at least about 2050 μg, at least about 2060 μg, at least about 2070 μg, at least about 2080 μg, at least about 2090 μg, at least about 2100 μg, at least about 2110 μg, at least about 2120 μg, at least about 2130 μg, at least about 2140 μg, at least about 2150 μg, at least about 2160 μg, at least about 2170 μg, at least about 2180 μg, at least about 2190 μg, at least about 2200 μg, at least about 2210 μg, at least about 2220 μg, at least about 2230 μg, at least about 2240 μg, at least about 2250 μg, at least about 2260 μg, at least about 2270 μg, at least about 2280 μg, at least about 2290 μg, at least about 2300 μg, at least about 2310 μg, at least about 2320 μg, at least about 2330 μg, at least about 2340 μg, at least about 2350 μg, at least about 2360 μg, at least about 2370 μg, at least about 2380 μg, at least about 2390 μg, at least about 2400 μg, at least about 2410 μg, at least about 2420 μg, at least about 2430 μg, at least about 2440 μg, at least about 2450 μg, at least about 2460 μg, at least about 2470 μg, at least about 2480 μg, at least about 2490 μg, at least about 2500 μg, or at least about 3000 μg.

In some embodiments, a dosing regimen can include administration of a maximal dose or dosing frequency that avoids significant undesirable side effects. In some embodiments, a total daily dose may be at least 50 μg, at least 100 μg, at least 300 μg, at least 600 μg, at least 900 μg, at least 1800 μg, at least 2500 μg, or at least 3000 μg.

In some embodiments, a dosing regimen can include administration of a maximal dose or dosing frequency that avoids significant undesirable side effects. In some embodiments, a total daily dose can consist of at least 50 μg, at least 100 μg, at least 300 μg, at least 600 μg, at least 900 μg, at least 1800 μg, at least 2500 μg, or at least 3000 μg.

In some embodiments, a dose of a peptide of the present disclosure, or a pharmaceutical composition thereof, administered to a subject in need thereof, is about 0.5 μg/kg, about 1.25 μg/kg, about 3.5 μg/kg, about 7.5 μg/kg, about 11 μg/kg, about 22 μg/kg, about 31 μg/kg, or about 37 μg/kg of the subject's body weight.

In some embodiments, a dose of a peptide of the present disclosure, or a pharmaceutical composition thereof, administered to a subject in need thereof, is about 0.5 μg/kg to about 37 μg/kg; about 1 μg/kg to about 37 μg/kg; about 1.25 μg/kg to about 37 μg/kg; about 3.5 μg/kg to about 37 μg/kg; about 7.5 μg/kg to about 37 μg/kg; about 11 μg/kg to about 37 μg/kg; about 0.5 μg/kg to about 37 μg/kg; about 0.5 μg/kg to about 31 μg/kg; about 0.5 μg/kg to about 22 μg/kg; about 0.5 μg/kg to about 11 μg/kg; about 0.5 μg/kg to about 7.5 μg/kg; about 0.5 μg/kg to about 3.5 μg/kg; or about 0.5 μg/kg to about 1.25 μg/kg, of the subject's body weight.

In some embodiments, a dose of a peptide of the present disclosure, or a pharmaceutical composition thereof, administered to a subject in need thereof, is about 0.25 μg/kg; about 0.5 μg/kg; about 0.75 μg/kg; about 1 μg/kg; about 1.25 μg/kg; about 1.5 μg/kg; about 1.75 μg/kg; about 2 μg/kg; about 2.25 μg/kg; about 2.5 μg/kg; about 2.75 μg/kg; about 3 μg/kg; about 3.25 μg/kg; about 3.5 μg/kg; about 3.75 μg/kg; about 4 μg/kg; about 4.25 μg/kg; about 4.5 μg/kg; about 4.75 μg/kg; about 5 μg/kg; about 5.25 μg/kg; about 5.5 μg/kg; about 5.75 μg/kg; about 6 μg/kg; about 6.25 μg/kg; about 6.5 μg/kg; about 6.75 μg/kg; about 7 μg/kg; about 7.25 μg/kg; about 7.5 μg/kg; about 7.75 μg/kg; about 8 μg/kg; about 8.25 μg/kg; about 8.5 μg/kg; about 8.75 μg/kg; about 9 μg/kg; about 9.25 μg/kg; about 9.5 μg/kg; about 9.75 μg/kg; about 10 μg/kg; about 10.25 μg/kg; about 10.5 μg/kg; about 10.75 μg/kg; about 11 μg/kg; about 11.25 μg/kg; about 11.5 μg/kg; about 11.75 μg/kg; about 12 μg/kg; about 12.25 μg/kg; about 12.5 μg/kg; about 12.75 μg/kg; about 13 μg/kg; about 13.25 μg/kg; about 13.5 μg/kg; about 13.75 μg/kg; about 14 μg/kg; about 14.25 μg/kg; about 14.5 μg/kg; about 14.75 μg/kg; about 15 μg/kg; about 15.25 μg/kg; about 15.5 μg/kg; about 15.75 μg/kg; about 16 μg/kg; about 16.25 μg/kg; about 16.5 μg/kg; about 16.75 μg/kg; about 17 μg/kg; about 17.25 μg/kg; about 17.5 μg/kg; about 17.75 μg/kg; about 18 μg/kg; about 18.25 μg/kg; about 18.5 μg/kg; about 18.75 μg/kg; about 19 μg/kg; about 19.25 μg/kg; about 19.5 μg/kg; about 19.75 μg/kg; about 20 μg/kg; about 20.25 μg/kg; about 20.5 μg/kg; about 20.75 μg/kg; about 21 μg/kg; about 21.25 μg/kg; about 21.5 μg/kg; about 21.75 μg/kg; about 22 μg/kg; about 22.25 μg/kg; about 22.5 μg/kg; about 22.75 μg/kg; about 23 μg/kg; about 23.25 μg/kg; about 23.5 μg/kg; about 23.75 μg/kg; about 24 μg/kg; about 24.25 μg/kg; about 24.5 μg/kg; about 24.75 μg/kg; about 25 μg/kg; about 25.25 μg/kg; about 25.5 μg/kg; about 25.75 μg/kg; about 26 μg/kg; about 26.25 μg/kg; about 26.5 μg/kg; about 26.75 μg/kg; about 27 μg/kg; about 27.25 μg/kg; about 27.5 μg/kg; about 27.75 μg/kg; about 28 μg/kg; about 28.25 μg/kg; about 28.5 μg/kg; about 28.75 μg/kg; about 29 μg/kg; about 29.25 μg/kg; about 29.5 μg/kg; about 29.75 μg/kg; about 30 μg/kg; about 30.25 μg/kg; about 30.5 μg/kg; about 30.75 μg/kg; about 31 μg/kg; about 31.25 μg/kg; about 31.5 μg/kg; about 31.75 μg/kg; about 32 μg/kg; about 32.25 μg/kg; about 32.5 μg/kg; about 32.75 μg/kg; about 33 μg/kg; about 33.25 μg/kg; about 33.5 μg/kg; about 33.75 μg/kg; about 34 μg/kg; about 34.25 μg/kg; about 34.5 μg/kg; about 34.75 μg/kg; about 35 μg/kg; about 35.25 μg/kg; about 35.5 μg/kg; about 35.75 μg/kg; about 36 μg/kg; about 36.25 μg/kg; about 36.5 μg/kg; about 36.75 μg/kg; about 37 μg/kg; about 37.25 μg/kg; about 37.5 μg/kg; about 37.75 μg/kg; about 38 μg/kg; about 38.25 μg/kg; about 38.5 μg/kg; about 38.75 μg/kg; about 39 μg/kg; about 39.25 μg/kg; about 39.5 μg/kg; about 39.75 μg/kg; about 40 μg/kg, of the subject's body weight.

In some embodiments, a dose of a peptide of the present disclosure, or a pharmaceutical composition thereof, administered to a subject in need thereof, is about 2 μg/kg; about 2.1 μg/kg; about 2.2 μg/kg; about 2.3 μg/kg; about 2.4 μg/kg; about 2.5 μg/kg; about 2.6 μg/kg; about 2.7 μg/kg; about 2.8 μg/kg; about 2.9 μg/kg; about 3 μg/kg; about 3.1 μg/kg; about 3.2 μg/kg; about 3.3 μg/kg; about 3.4 μg/kg; about 3.5 μg/kg; about 3.6 μg/kg; about 3.7 μg/kg; about 3.8 μg/kg; about 3.9 μg/kg; about 4 μg/kg; about 4.1 μg/kg; about 4.2 μg/kg; about 4.3 μg/kg; about 4.4 μg/kg; about 4.5 μg/kg; about 4.6 μg/kg; about 4.7 μg/kg; about 4.8 μg/kg; about 4.9 μg/kg; about 5 μg/kg; about 5.1 μg/kg; about 5.2 μg/kg; about 5.3 μg/kg; about 5.4 μg/kg; about 5.5 μg/kg; about 5.6 μg/kg; about 5.7 μg/kg; about 5.8 μg/kg; about 5.9 μg/kg; about 6 μg/kg; about 6.1 μg/kg; about 6.2 μg/kg; about 6.3 μg/kg; about 6.4 μg/kg; about 6.5 μg/kg; about 6.6 μg/kg; about 6.7 μg/kg; about 6.8 μg/kg; about 6.9 μg/kg; about 7 μg/kg; about 7.1 μg/kg; about 7.2 μg/kg; about 7.3 μg/kg; about 7.4 μg/kg; about 7.5 μg/kg; about 7.6 μg/kg; about 7.7 μg/kg; about 7.8 μg/kg; about 7.9 μg/kg; about 8 μg/kg; about 8.1 μg/kg; about 8.2 μg/kg; about 8.3 μg/kg; about 8.4 μg/kg; about 8.5 μg/kg; about 8.6 μg/kg; about 8.7 μg/kg; about 8.8 μg/kg; about 8.9 μg/kg; about 9 μg/kg; about 9.1 μg/kg; about 9.2 μg/kg; about 9.3 μg/kg; about 9.4 μg/kg; about 9.5 μg/kg; about 9.6 μg/kg; about 9.7 μg/kg; about 9.8 μg/kg; about 9.9 μg/kg; about 10 μg/kg; about 10.1 μg/kg; about 10.2 μg/kg; about 10.3 μg/kg; about 10.4 μg/kg; about 10.5 μg/kg; about 10.6 μg/kg; about 10.7 μg/kg; about 10.8 μg/kg; about 10.9 μg/kg; about 11 μg/kg; about 11.1 μg/kg; about 11.2 μg/kg; about 11.3 μg/kg; about 11.4 μg/kg; about 11.5 μg/kg; about 11.6 μg/kg; about 11.7 μg/kg; about 11.8 μg/kg; about 11.9 μg/kg; about 12 μg/kg, of the subject's body weight.

In another embodiment, a dose of the present disclosure to be administered to a subject in need thereof can be administered as a rectal formulation. In some embodiments, the rectal formulation can be a 20 mL solution. In some embodiments, the total daily dose of the 20 mL rectal formulation can be at least 5.00 μg/mL, 15.0 μg/mL, 30.0 μg/mL, 45.0 μg/mL, 90.0 μg/mL, or 125 μg/mL, or any dosage amount there between.

It is understood that the amount of peptide dosed per day may be administered, in non-limiting examples, every day, every other day, every 2 days, every 3 days, every 4 days, or every 5 days. For example, with every other day administration, a dose may be initiated on Monday with a first subsequent dose administered on Wednesday, a second subsequent dose administered on Friday, and so on.

In the case wherein the subject's status does improve, upon the doctor's discretion the administration of the peptide of the invention, or a pharmaceutical composition thereof, is optionally given continuously; alternatively, the dose of drug being administered can be temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Depending on the severity of the condition, and the various factors discussed herein, the dose, frequency and the duration of the treatment can be adjusted accordingly, in view of proper medical standards known to those of skill in the art. In certain embodiments, the peptides of the invention, or a pharmaceutical composition thereof, can be administered as an initial dose of at least about 100 μg, at least about 300 μg, at least about 600 μg, at least about 900 μg, at least about 1800 μg, at least about 2500 μg, or at least about 3000 μg.

In some embodiments, the first dose may be an initial loading dose, to be followed subsequently by a plurality of maintenance doses. In certain embodiments, the initial dose may be followed by administration of a second or a plurality of subsequent doses of the compounds of the present disclosure in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks, or doses of the combination of the disclosure may be repeated and the administrations may be separated by at least 1 day, 2 days, 3 days, 5 days, 10 days, 15 days, 30 days, 45 days, 2 months, 75 days, 3 months, or at least 6 months.

Once improvement of the subject's conditions has occurred, a maintenance dose may be administered if necessary. Subsequently, the dosage or the frequency of administration, or both, is reduced, as a function of the subject's pain, to a level at which the improved disease is retained. In certain embodiments, subjects require intermittent treatment on a long-term basis upon any recurrence of symptoms.

In some embodiments, dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic peptide of the invention is from about 50 μg to about 5000 μg. One of ordinary skill in the art would be able to study the relevant factors and make the determination regarding the effective amount of the therapeutic compound without undue experimentation.

In some embodiments, a dose of the peptide of the present disclosure to be administered to a subject in need thereof can include a single dose of about 100 μg, to about 300 μg.

In some embodiments, the selected dosage level depends upon a variety of factors including the activity of the particular peptide employed, the time of administration, the rate of excretion, the duration of the treatment, other drugs, compounds or materials used in combination with the compound, the age, sex, weight, condition, general health and prior medical history of the patient being treated, and like factors well, known in the medical arts.

In some embodiments, an effective amount of a peptide of the present disclosure, or a pharmaceutical composition thereof, for use in therapy is an amount sufficient to treat or prevent a visceral pain condition (e.g., bladder pain associated with interstitial cystitis/bladder pain syndrome (IC/BPS); pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology, diverticulitis pain, pain associated with gastrointestinal disorders, pain associated with venereal diseases, pain associated with irritable bowel syndrome (IBS), rectal pain, chronic proctalgia, proctalgia fugax, anal pain, chronic anal fissure, post-operative anal pain, pain associated with cancer, pain associated with gastrointestinal tract neoplasms, general pelvic pain, orchialgia, chronic prostatitis, prostatodynia, vulvodynia, urethral syndrome, penile pain, perianal pain, and pain associated with ulcerative colitis, ulcerative proctitis, or Crohn's disease), such as those described herein, reduce the symptoms associated with the a visceral pain condition.

In some embodiments, the size of the dose for therapeutic and/or prophylactic purposes of a peptide of the present disclosure, or a pharmaceutical composition thereof, will naturally vary according to one or more of the following variables: the nature and severity of the conditions; the age and sex of the animal or patient; and/or the route of administration, according to well-known principles of medicine.

In some embodiments, one or more of the peptides of the present disclosure can be combined with one or more excipients to produce a single unit dosage form. The amount of the active ingredient (i.e., peptides of the present disclosure) that is combined with one or more excipients to produce a single unit dosage form will necessarily vary depending upon the individual treated and the particular route of administration. For example, a formulation intended for rectal administration to humans will generally contain, e.g., from about 50 μg to about 5000 μg of active agent (more suitably from 100 μg to 2500 μg, e.g., at least about 100 μg, at least about 300 μg, at least about 600 μg, at least about 900 μg, at least about 1800 μg, or at least about 2500 μg) compounded with an appropriate and convenient amount of excipients which may vary from about 5% to about 99% by weight of the total composition.

### Rectal Dosing

In some embodiments, part or all of the dose may be administered by a rectal bolus. For example, in some embodiments, the dose may be from about from about 50 μg to about 5000 μg of active agent, more suitably from 100 to 2500 μg, e.g., at least about 100 μg, at least about 300 μg, at least about 600 μg, at least about 900 μg, at least about 1800 μg, at least about 2500 μg, or at least about 3000 μg for rectal administration.

In some embodiments, the rectal dose may be administered in a pharmaceutical composition formulated as an ointment; a suppository; an enema solution; a hydrogel; a murphy drip; or a rectal foam.

In some embodiments, the rectal dose may be administered as a foam, wherein the dose of the peptide of the present disclosure, or a pharmaceutically acceptable salt, hydrate, or solvate thereof, comprises, consists essentially of, or consists of, a dose of the peptide of the present disclosure ranging from about 0.0005% to about 5%; from about 0.0006% to about 5%; from about 0.0007% to about 5%; from about 0.0008% to about 5%; from about 0.0009% to about 5%; from about 0.001% to about 5%; from about 0.0015% to about 5%; from about 0.002% to about 5%; from about 0.0025% to about 5%; from about 0.003% to about 5%; from about 0.0035% to about 5%; from about 0.004% to about 5%; from about 0.0045% to about 5%; from about 0.005% to about 5%; from about 0.0055% to about 5%; from about 0.006% to about 5%; from about 0.0065% to about 5%; from about 0.007% to about 5%; from about 0.0075% to about 5%; from about 0.008% to about 5%; from about 0.0085% to about 5%; from about 0.009% to about 5%; from about 0.0095% to about 5%; from about 0.01% to about 5%; from about 0.02% to about 5%; from about 0.03% to about 5%; from about 0.04% to about 5%; from about 0.05% to about 5%; from about 0.1% to about 5%; from about 0.15% to about 5%; from about 0.2% to about 5%; from about 0.25% to about 5%; from about 0.3% to about 5%; from about 0.35% to about 5%; from about 0.4% to about 5%; from about 0.45% to about 5%; from about 0.5% to about 5%; from about 0.55% to about 5%; from about 0.6% to about 5%; from about 0.65% to about 5%; from about 0.7% to about 5%; from about 0.75% to about 5%; from about 0.8% to about 5%; from about 0.85% to about 5%; from about 0.9% to about 5%; from about 0.95% to about 5%; from about 1% to about 5%; from about 1.05% to about 5%; from about 1.1% to about 5%; from about 1.15% to about 5%; from about 1.2% to about 5%; from about 1.25% to about 5%; from about 1.3% to about 5%; from about 1.35% to about 5%; from about 1.4% to about 5%; from about 1.45% to about 5%; from about 1.5% to about 5%; from about 1.55% to about 5%; from about 1.6% to about 5%; from about 1.65% to about 5%; from about 1.7% to about 5%; from about 1.75% to about 5%; from about 1.8% to about 5%; from about 1.85% to about 5%; from about 1.9% to about 5%; from about 1.95% to about 5%; from about 2% to about 5%; from about 2.05% to about 5%; from about 2.1% to about 5%; from about 2.15% to about 5%; from about 2.2% to about 5%; from about 2.25% to about 5%; from about 2.3% to about 5%; from about 2.35% to about 5%; from about 2.4% to about 5%; from about 2.45% to about 5%; from about 2.5% to about 5%; from about 2.55% to about 5%; from about 2.6% to about 5%; from about 2.65% to about 5%; from about 2.7% to about 5%; from about 2.75% to about 5%; from about 2.8% to about 5%; from about 2.85% to about 5%; from about 2.9% to about 5%; from about 2.95% to about 5%; from about 3% to about 5%; from about 3.05% to about 5%; from about 3.1% to about 5%; from about 3.15% to about 5%; from about 3.2% to about 5%; from about 3.25% to about 5%; from about 3.3% to about 5%; from about 3.35% to about 5%; from about 3.4% to about 5%; from about 3.45% to about 5%; from about 3.5% to about 5%; from about 3.55% to about 5%; from about 3.6% to about 5%; from about 3.65% to about 5%; from about 3.7% to about 5%; from about 3.75% to about 5%; from about 3.8% to about 5%; from about 3.85% to about 5%; from about 3.9% to about 5%; from about 3.95% to about 5%; from about 4% to about 5%; from about 4.05% to about 5%; from about 4.1% to about 5%; from about 4.15% to about 5%; from about 4.2% to about 5%; from about 4.25% to about 5%; from about 4.3% to about 5%; from about 4.35% to about 5%; from about 4.4% to about 5%; from about 4.45% to about 5%; from about 4.5% to about 5%; from about 4.55% to about 5%; from about 4.6% to about 5%; from about 4.65% to about 5%; from about 4.7% to about 5%; from about 4.75% to about 5%; from about 4.8% to about 5%; from about 4.85% to about 5%; from about 4.9% to about 5%; or from about 4.95% to about 5%; w/w % of the total composition.

In some embodiments, the rectal dose may be administered as a foam, wherein the dose of the peptide of the present disclosure, or a pharmaceutically acceptable salt, hydrate, or solvate thereof, comprises, consists essentially of, or consists of, a dose of the peptide of the present disclosure ranging from about 0.005% to about 0.5%; from about 0.015% to about 0.5%; from about 0.025% to about 0.5%; from about 0.035% to about 0.5%; from about 0.045% to about 0.5%; from about 0.055% to about 0.5%; from about 0.065% to about 0.5%; from about 0.075% to about 0.5%; from about 0.085% to about 0.5%; from about 0.095% to about 0.5%; from about 0.105% to about 0.5%; from about 0.115% to about 0.5%; from about 0.125% to about 0.5%; from about 0.135% to about 0.5%; from about 0.145% to about 0.5%; from about 0.155% to about 0.5%; from about 0.165% to about 0.5%; from about 0.175% to about 0.5%; from about 0.185% to about 0.5%; from about 0.195% to about 0.5%; from about 0.205% to about 0.5%; from about 0.215% to about 0.5%; from about 0.225% to about 0.5%; from about 0.235% to about 0.5%; from about 0.245% to about 0.5%; from about 0.255% to about 0.5%; from about 0.265% to about 0.5%; from about 0.275% to about 0.5%; from about 0.285% to about 0.5%; from about 0.295% to about 0.5%; from about 0.305% to about 0.5%; from about 0.315% to about 0.5%; from about 0.325% to about 0.5%; from about 0.335% to about 0.5%; from about 0.345% to about 0.5%; from about 0.355% to about 0.5%; from about 0.365% to about 0.5%; from about 0.375% to about 0.5%; from about 0.385% to about 0.5%; from about 0.395% to about 0.5%; from about 0.405% to about 0.5%; from about 0.415% to about 0.5%; from about 0.425% to about 0.5%; from about 0.435% to about 0.5%; from about 0.445% to about 0.5%; from about 0.455% to about 0.5%; from about 0.465% to about 0.5%; from about 0.475% to about 0.5%; from about 0.485% to about 0.5%; or from about 0.495% to about 0.5%, w/w % of the total composition.

In some embodiments, the rectal dose may be administered as a foam, wherein the dose of the peptide of the present disclosure, or a pharmaceutically acceptable salt, hydrate, or solvate thereof, comprises, consists essentially of, or consists of, a dose of the peptide of the present disclosure is about 0.00870%, w/w % of the total composition.

In some embodiments, the rectal dose may be administered as a foam, wherein the dose of the peptide of the present disclosure, or a pharmaceutically acceptable salt, hydrate, or solvate thereof, comprises, consists essentially of, or consists of, a dose of the peptide of the present disclosure is about 0.00961%, w/w % of the total composition.

In some embodiments, the rectal dose may be administered as a foam, wherein the dose of the peptide of the present disclosure, or a pharmaceutically acceptable salt, hydrate, or solvate thereof, comprises, consists essentially of, or consists of, a dose of the peptide of the present disclosure is about 0.0261%, w/w % of the total composition.

In some embodiments, the rectal dose may be administered as a foam, wherein the dose of the peptide of the present disclosure, or a pharmaceutically acceptable salt, hydrate, or solvate thereof, comprises, consists essentially of, or consists of, a dose of the peptide of the present disclosure is about 0.0288%, w/w % of the total composition.

In some embodiments, the rectal dose may be administered as a foam, wherein the dose of the peptide of the present disclosure, or a pharmaceutically acceptable salt, hydrate, or solvate thereof, comprises, consists essentially of, or consists of, a dose of the peptide of the present disclosure is about 0.301%, w/w % of the total composition.

In some embodiments, the rectal dose may be administered as a foam, wherein the dose of the peptide of the present disclosure, or a pharmaceutically acceptable salt, hydrate, or solvate thereof, comprises, consists essentially of, or consists of, a dose of the peptide of the present disclosure is about 0.335%, w/w % of the total composition.

In some embodiments, the rectal dose may be administered as a foam, e.g., a pharmaceutical rectal foam composition, wherein the pharmaceutical rectal foam composition comprises a dose of the peptide of the present disclosure of about 100 μg, to about 300 μg.

In some embodiments, the rectal dose may be administered as a foam, e.g., a pharmaceutical rectal foam composition, wherein the pharmaceutical rectal foam composition comprises a dose of the peptide of the present disclosure ranging from about 50 μg, to about 400 μg.

In some embodiments, the rectal dose may be administered as a foam, e.g., a pharmaceutical rectal foam composition, wherein the pharmaceutical rectal foam composition comprises a dose of the peptide of the present disclosure ranging from about 100 μg, to about 300 μg.

In some embodiments, the rectal dose may be administered as a foam, e.g., a pharmaceutical rectal foam composition, wherein the pharmaceutical rectal foam composition comprises a dose of the peptide of the present disclosure ranging from about 100 μg, to about 300 μg, wherein the dose is administered once or more times per day, and/or one or more times per week, for example, for one to four weeks, or one to eight weeks, or one to twelve weeks, or one to fourteen weeks.

In some embodiments, the rectal dose may be administered as a foam, e.g., a pharmaceutical rectal foam composition, wherein the pharmaceutical rectal foam composition comprises a dose of the peptide of the present disclosure of about 100 μg, wherein the foam comprises a unit dose strength/dose volume of about 100 μg/1.15 mL (foaming to 20 mL).

In some embodiments, the rectal dose may be administered as a foam, e.g., a pharmaceutical rectal foam composition, wherein the pharmaceutical rectal foam composition comprises a dose of the peptide of the present disclosure is about 300 μg, wherein the foam comprises a unit dose strength/dose volume of about 300 μg/1.15 mL (foaming to 20 mL).

In some embodiments, the rectal dose may be administered as a foam, e.g., a pharmaceutical rectal foam composition, wherein the pharmaceutical rectal foam composition comprises a dose of the peptide of the present disclosure of about 100 μg, wherein the foam comprises a unit dose strength/dose volume of about 100 μg/1.15 mL (foaming to 20 mL); wherein the dosage level is about 100 μg once daily.

In some embodiments, the rectal dose may be administered as a foam, e.g., a pharmaceutical rectal foam composition, wherein the pharmaceutical rectal foam composition comprises a dose of the peptide of the present disclosure is about 300 μg, wherein the foam comprises a unit dose strength/dose volume of about 300 μg/1.15 mL (foaming to 20 mL); wherein the dosage level is about 300 μg once daily.

### Verifying Outcomes

### Efficacy of Therapies

In some embodiments, the peptide of the present disclosure and pharmaceutical compositions thereof, and/or methods of treatments of the present disclosure and therapies described herein, can be tested for their ability to accomplish the following: reduce visceral pain; a reversal of persistent afferent bladder hypersensitivity; a reduction in bladder pain associated with IC/BPS; a reduction in chronic pelvic pain; a reduction in endometriosis associated pain; a reduction in pain associated with radiation proctopathy; a reduction in vaginal hypersensitivity associated with vaginal irritation; and/or a reduction in allodynia-associated pain; using standard techniques.

### Identification and Assessment of Subjects Prior to Treatment

As used herein, the terms “patient” and “subject” are used interchangeably.

Prior to commencement of any of the treatments or therapies described herein, using the peptide of the present disclosure or a pharmaceutical composition thereof, several measures known in the art can be used to first identify and/or classify the patients and/or their conditions.

For example, in some embodiments, a patient's overall quality of life can be assessed, for example, using the McGill Quality of Life Questionnaire (MQOL) (Cohen et al (1995) Palliative Medicine 9: 207-219). The MQOL measures physical symptoms; physical, psychological and existential well-being; support; and overall quality of life. To assess symptoms such as nausea, mood, appetite, insomnia, mobility and fatigue the Symptom Distress Scale (SDS) developed by McCorkle and Young ((1978) Cancer Nursing 1: 373-378) can be used.

Patients can also be classified according to the type and/or stage of their disease and/or by amount of pain (e.g., type or amount of visceral pain).

In some embodiments, patient pain, e.g., in the case of patients suffering from IC/PBS, can be based on a symptom-based diagnosis. In some embodiments, patients with IC/BPS can be assessed using the Interstitial Cystitis Symptom Index (ICSI), also known as the O'Leary-Sant Symptom Index.

In some embodiments, patients with IC/BPS can be assessed based on bladder pain, urgency, frequency, and nocturia, and the frequency and/or severity thereof.

In some embodiments, patients with IC/BPS can be assessed based on one or more of the following symptoms: bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; or Hunner's lesions (mucosal lesions or ulcerations seen with or without hydrodistension of the bladder) associated with IC/BPS.

In some embodiments, a subject in need of treatment of the methods and therapies of the present disclosure can be identified based on one or more symptoms experienced by the subject. For example, in some embodiments, a subject in need of treatment can be identified based one or more symptoms of IC/BPS.

In some embodiments, a subject in need of treatment can be identified based on the subject's bladder pain.

In some embodiments, a subject in need of treatment can be identified based on the subject's increased urinary urgency, increased frequency, and/or nocturia.

In some embodiments, a subject in need of treatment can be identified based on the subject's overall daily bladder pain overall (which can include sensations like burning, pressure, and/or discomfort).

In some embodiments, a subject in need of treatment can be identified based on the subject's weekly average of a burning sensation in the bladder at its worst.

In some embodiments, a subject in need of treatment can be identified based on the subject's weekly average of a pressure sensation in the bladder at its worst.

In some embodiments, a subject in need of treatment can be identified based on the subject's weekly average of discomfort in the bladder at its worst.

In some embodiments, a subject in need of treatment can be identified based on the subject's level of pain according to the Genitourinary Pain Index (GUPI) Pain subscale.

In some embodiments, a subject in need of treatment can be identified based on the subject's weekly average of bladder pain-free days.

In some embodiments, a subject in need of treatment can be identified based on the subject's increase in urinary urgency relative to baseline.

In some embodiments, a subject in need of treatment can be identified based on the subject's increase in urinary frequency relative to baseline.

In some embodiments, a subject in need of treatment can be identified based on the subject's mean nighttime voiding frequency (nocturia).

In some embodiments, a subject in need of treatment can be identified based on the subject's score as recorded in the O'Leary/Sant Interstitial Cystitis Symptom Index (ICSI) total score, relative to baseline.

In some embodiments, a subject in need of treatment can be identified based on the subject's score in the Genitourinary Pain Index (GUPI) Urinary subscale score, relative to baseline.

In some embodiments, a subject in need of treatment can be identified based on the subject's score in the GUPI Quality of life (QoL) subscale score, relative to baseline.

In some embodiments, a subject in need of treatment can be identified based on the subject's based on a change in the Global Response Assessment (GRA) total score, relative to baseline.

In some embodiments, a subject in need of treatment can be identified based on the subject's change in Sexual Function Question score, relative to baseline.

In some embodiments, a subject in need of treatment can be identified based on the subject's sleep difficulty due to bladder pain, relative to baseline.

In some embodiments, a subject in need of treatment can be identified based on the subject's sleep difficulty due to needing to urinate, relative to baseline.

In some embodiments, a subject in need of treatment can be identified based on the subject's change in the body pain category as assessed using the Margolis body map, relative to baseline.

In some embodiments, a subject in need of treatment can be identified based on the subject's change from baseline in IC/BPS Symptom Severity.

In some embodiments, a subject in need of treatment can be identified based on the subject's IC/BPS global impression of symptom change, relative to baseline.

In some embodiments, a subject in need of treatment can be identified based on the subject's change from baseline in EuroQol 5 Dimension 5-Level Version (EQ-5D-5L) utility score and Visual Analog Scale.

In some embodiments, a subject in need of treatment can be identified based on the subject's change from baseline in 16s RNA based analysis of gut microbiome composition at the genus level.

In some embodiments, a subject in need of treatment can be identified based on the subject's change in one or more of the following symptoms: bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; or Hunner's lesions (mucosal lesions or ulcerations seen with or without hydrodistension of the bladder) associated with IC/BPS.

In some embodiments, a subject in need of treatment can be identified based on the subject's cystoscopy findings, which may or may not show bladder inflammation, including Hunner's lesions (mucosal lesions or ulcerations seen with or without hydrodistension of the bladder), or other pathology. Diagnosis is generally not made until other diseases that could cause these symptoms are ruled out.

In some embodiments, a subject in need of treatment can be identified based on the subject's use of pentosan polysulfate. Pentosan polysulfate is a synthetic sulfated polysaccharide used for bladder pain.

In some embodiments, a subject in need of treatment can be identified based on the subject's prior treatment with one or more therapies to relieve one or more symptoms a visceral pain as described herein. For example, without limitation, in some embodiments, a subject in need of treatment can be identified based on the subject's prior treatment of intravesical installations, in an attempt to relieve BPS symptoms with or without dimethyl sulfoxide (DMSO).

In some embodiments, a subject in need of treatment can be identified based on the subject's increase in redness, rectal pain, bleeding, and/or mucus as observed on visual inspection of the rectal (perianal) area.

In some embodiments, a subject in need of treatment can be identified based on the subject's anoscopy findings, performed alone or in concert with a rectal examination.

In some embodiments, a subject in need of treatment can be identified based on one or more of the following urinary symptoms related to IC/BPS that as occurred, one, two, three, or more months: nocturia, wherein the subject voids two or more times per night; a daytime urinary frequency of greater than 8 times per day; or any feeling of urinary urgency.

Exemplary descriptions of patient assessment for IC/BPS is provided in Diggs et al., Assessing urgency in interstitial cystitis/painful bladder syndrome. Urology. 2007 February; 69(2): 210-214, the disclosure of which is incorporated herein by reference in its entirety.

### Monitoring of Patient Outcome

The endpoint of a treatment is a measurable outcome that indicates the effectiveness of a treatment under evaluation. The endpoint is established prior to the commencement of the treatment and will vary depending on the type of treatment pursued. Examples of endpoints include, for example, the reduction in degree and/or frequency of one or more of the following: visceral pain; persistent afferent bladder hypersensitivity; bladder pain associated with IC/BPS; chronic pelvic pain; endometriosis associated pain; pain associated with radiation proctopathy; vaginal hypersensitivity associated with vaginal irritation; and/or a allodynia-associated pain. Other endpoints include toxicity and quality of life.

Methods of determining patient outcome are well known in the art. For example, in some embodiments, patient outcome can be determined using the Genitourinary Pain Index (GUPI) Pain subscale.

In some embodiments, patient outcome can be determined based on O'Leary/Sant Interstitial Cystitis Symptom Index (ICSI) total score.

In some embodiments, patient outcome can be determined using the Global Response Assessment (GRA) total score.

In some embodiments, patient outcome can be determined using the Sexual Function Question score.

In some embodiments, patient outcome can be determined based on the patient's sleep difficulty due to bladder pain.

In some embodiments, patient outcome can be determined based on the patient's sleep difficulty due to needing to urinate.

In some embodiments, patient outcome can be determined using the Margolis body map (body pain).

In some embodiments, patient outcome can be determined based on the degree of reduction in IC/BPS symptom severity.

In some embodiments, patient outcome can be determined based on the degree of reduction in IC/BPS global impression of symptom change.

In some embodiments, patient outcome can be determined based on the EuroQol 5 Dimension 5-Level Version (EQ-5D-5L) utility score and/or Visual Analog Scale.

In some embodiments, patient outcome can be determined based on 16s RNA based analysis of gut microbiome composition at the genus level.

In some embodiments, patient outcome can be determined based on pharmacokinetic parameters of the peptide of the present disclosure and metabolites in the plasma of the patient.

Any of the foregoing outcome methods can be evaluated using the methods described herein, e.g., the questionnaires described herein and/or the use of daily and/or weekly electronic diary (eDiary) questions; all of which are likewise known in the art.

In some embodiments, endpoints can be based on factors such as peptide bioactivity and the like.

In some embodiments, 16s RNA based analysis of gut microbiome composition at the genus level can be used to evaluate patient outcome.

Methods of analyzing the gut microbiome are described in Mas-Lloret et al., Gut microbiome diversity detected by high-coverage 16S and shotgun sequencing of paired stool and colon sample. Sci Data. 2020 Mar. 16; 7(1):92; Wei et al., 16S rRNA gene amplicon sequencing of gut microbiota in gestational diabetes mellitus and their correlation with disease risk factors. J Endocrinol Invest. 2022; 45(2): 279-289; Almonacid et al., 16S rRNA gene sequencing and healthy reference ranges for 28 clinically relevant microbial taxa from the human gut microbiome. PLOS One. 2017; 12(5): e0176555; Liu et al., Leveraging 16S IRNA Microbiome Sequencing Data to Identify Bacterial Signatures for Irritable Bowel Syndrome. Front Cell Infect Microbiol. 2021 Jun. 11; 11:645951; and Duan et al., Alterations of Gut Microbiota in Patients With Irritable Bowel Syndrome Based on 16S rRNA-Targeted Sequencing: A Systematic Review. Clin Transl Gastroenterol. 2019 February; 10(2):e00012; the disclosures of which are incorporated herein by reference in their entireties.

Several methods can be used for evaluating the bioactivity of the peptide of the present disclosure, or pharmaceutical compositions thereof, including, but not limited to, immunoassays (e.g., enzyme-linked immunosorbent assay), radioimmuno assays, immunoradiometric assays, gel electrophoresis (e.g., SDS-PAGE), high performance liquid chromatography (HPLC), and/or high performance capillary electrophoresis (HPCE). In some embodiments, the bioactivity of the peptide of the present disclosure, or pharmaceutical compositions thereof, can be assessed by a method comprising fixing the peptides, incubating peptides with guanylate cyclase C (GC-C), incubating GC-C bound peptides with antibodies against GC-C, incubating GC-C antibody-bound peptides with fluorescently labeled antibodies against GC-C antibodies, and detecting the peptides bound to the GC-C antibodies by measuring the fluorescence intensity using a plate reader. The drug concentration can then be calculated based on the fluorescence reading of the solution.

For example, the bioactivity of the peptide of the present disclosure, or pharmaceutical compositions thereof, can be assessed and quantified using the following method, though other methods are available. The composition is added to a volumetric flask containing 60 mL of phosphate buffer having a pH of 4.5, and the flask is shaken for 60 minutes. 0.2 mL of the supernatant is then removed, and is added into one or more wells of a 96-well plate that is coated with GC-C receptors. The plate is sealed and incubated at 37° C. for 2 hr. At the end of incubation, the sample is removed and the plate is washed with phosphate buffered saline (PBS). The bound peptides are then incubated for 1 hour, at room temperature, with GC-C (such as is available from Sigma-Aldrich Inc.) labeled with fluorescein isocyanate (FITC) in blocking buffer. After incubation, the well is washed with PBS. The fluorescence intensity of the end product is detected, for example, by using a plate reader. The peptide concentration is then calculated based on the fluorescence reading of the solution.

### Response to Treatment and In Vivo Efficacy

In some embodiments, a patient can be evaluated during and/or after treatments of the present disclosure to determine whether said patient is responding and/or responsive to said treatment.

The terms “responsive” and “responsiveness,” as used herein, refer to the likelihood that a treatment (e.g., treatment with peptides of the present disclosure, or a pharmaceutical composition thereof) has (e.g., induces) a desired effect in a cell, a tissue, or a patient having a visceral pain condition (e.g., bladder pain associated with interstitial cystitis/bladder pain syndrome (IC/BPS); pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology, diverticulitis pain, pain associated with gastrointestinal disorders, pain associated with venereal diseases, pain associated with irritable bowel syndrome (IBS), rectal pain, chronic proctalgia, proctalgia fugax, anal pain, chronic anal fissure, post-operative anal pain, pain associated with cancer, pain associated with gastrointestinal tract neoplasms, general pelvic pain, orchialgia, chronic prostatitis, prostatodynia, vulvodynia, urethral syndrome, penile pain, perianal pain, and pain associated with ulcerative colitis, ulcerative proctitis, or Crohn's disease), such as those described herein. For example, the desired effect can include inhibition and/or a reduction in the amount of pain in a patient receiving treatment, by more than 1%, 2%, 3%, 4%, 5%, 6%, 7%, 8%, 9%, 10%, 20%, 30%, 40%, 50%, 60%, 70%, 80%, 90%, or 100%, relative to the amount of pain in a patient not exposed to the treatment, or the amount of pain experienced by the patient prior to administration of the peptide of the present disclosure. Responsiveness to treatment may be determined by methods known by those having ordinary skill in the art.

In some embodiments, the administration of a peptide of the present disclosure, a pharmaceutically acceptable salt, hydrate or solvate thereof, or a pharmaceutical composition comprising the same, provides at least a partial or complete response of the subjects.

In some embodiments, a partial response can be an inhibition and/or a reduction in the amount of pain in a patient receiving treatment of at least 0.01%, 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90%, 95%, 96%, 97%, 98%, or 99%, relative to the amount of pain in a patient not exposed to the treatment, or the amount of pain experienced by the patient prior to being administered a peptide of the present disclosure. In some embodiments, this response can be evident in at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90%, 95%, 96%, 97%, 98%, or 99% of the subjects treated.

The terms “responsive” and “responsiveness,” as used herein, refer to the likelihood that a treatment (e.g., treatment with peptides of the present disclosure, or a pharmaceutical composition thereof) has (e.g., induces) a desired effect in a cell, a tissue, or a patient having a visceral pain condition (e.g., bladder pain associated with interstitial cystitis/bladder pain syndrome (IC/BPS); pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology, diverticulitis pain, pain associated with gastrointestinal disorders, pain associated with venereal diseases, pain associated with irritable bowel syndrome (IBS), rectal pain, chronic proctalgia, proctalgia fugax, anal pain, chronic anal fissure, post-operative anal pain, pain associated with cancer, pain associated with gastrointestinal tract neoplasms, general pelvic pain, orchialgia, chronic prostatitis, prostatodynia, vulvodynia, urethral syndrome, penile pain, perianal pain, and pain associated with ulcerative colitis, ulcerative proctitis, or Crohn's disease), such as those described herein.

In some embodiments, a response to the methods of treatments described herein include inhibition and/or a reduction in the number or severity of symptoms in a patient receiving treatment, by more than 1%, 2%, 3%, 4%, 5%, 6%, 7%, 8%, 9%, 10%, 20%, 30%, 40%, 50%, 60%, 70%, 80%, 90%, or 100%, relative to the number or severity of symptoms in a patient prior to being exposed to the treatment; wherein the symptoms are selected from: bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; or any combination thereof.

In some embodiments, a partial response can be an inhibition and/or a reduction in the number or severity of symptoms associated with IC/BPS in a patient receiving treatment, wherein the inhibition and/or reduction is at least 0.01%, 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90%, 95%, 96%, 97%, 98%, or 99%, relative to the number or severity of symptoms associated with IC/BPS in the patient prior to being exposed to the treatment. In some embodiments, this response can be evident in at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90%, 95%, 96%, 97%, 98%, or 99% of the subjects treated.

In some embodiments, “reducing” or “inhibiting” or “limiting” or any variation of these terms, refers to making something (e.g., the number of symptoms, severity of symptoms, and/or frequency of symptoms, such as degree/severity of pain and/or frequency of pain) less in size, amount, intensity, or degree. For example, in some embodiments, the administration of a therapeutically effective amount of a peptide of the present disclosure and/or a pharmaceutical composition of the present disclosure, to a subject in need thereof, results in the following effect: a decrease in the frequency and/or severity of bladder pain associated with interstitial cystitis/bladder pain syndrome (IC/BPS); a decrease in the frequency and/or severity of urinary urgency associated with IC/BPS; a decrease in the frequency and/or severity of urinary frequency associated with IC/BPS; a decrease in the frequency and/or severity of nighttime voiding (nocturia) associated with IC/BPS; a decrease in the frequency and/or severity of burning sensation in the bladder associated with IC/BPS; a decrease in the frequency and/or severity of a burning sensation during urination associated with IC/BPS; a decrease in the frequency and/or severity of a pressure sensation in the bladder associated with IC/BPS; a decrease in the frequency and/or severity of discomfort in the bladder associated with IC/BPS; a decrease in the frequency and/or severity of bladder pain associated with IC/BPS; a decrease in the frequency and/or severity of urinary pain associated with IC/BPS; a decrease in the frequency and/or severity of genital pain associated with IC/BPS; a decrease in the frequency and/or severity of genitourinary pain associated with IC/BPS; a decrease in the frequency and/or severity of sleeping difficulties associated with IC/BPS; a decrease in the frequency and/or severity of body pain associated with IC/BPS; an increased quality of life associated with IC/BPS; a decrease in the frequency and/or severity of suicidal ideation that results from IC/BPS; a decrease in the frequency and/or severity of depression that results from IC/BPS; a reduction in the use of pain medication used by a subject to treat IC/BPS; a decrease in the frequency and/or severity of sexual dysfunction associated with IC/BPS; a decrease in the frequency and/or severity of loss of libido associated with IC/BPS; an increase in the ability to have sexual intercourse for a subject that previously did not have that ability because of one or more symptoms associated with IC/BPS; a decrease in the frequency and/or severity of bladder inflammation associated with IC/BPS; a decrease in the frequency and/or severity of Hunner's lesions (mucosal lesions or ulcerations seen with or without hydrodistension of the bladder) associated with IC/BPS; a decrease in the frequency and/or severity of pain of the abdominal region; a decrease in the frequency and/or severity of hypersensitivity of the bladder; a decrease in the frequency and/or severity of colonic pain; a decrease in the frequency and/or severity of extra-intestinal chronic pelvic pain; a decrease in the frequency and/or severity of endometriosis; a decrease in the frequency and/or severity of pain from excessive menstrual cramps; a decrease in the frequency and/or severity of pain during intercourse; a decrease in the frequency and/or severity of pain associated with endometriosis; a decrease in the frequency and/or severity of symptoms and/or pain associated with radiation proctopathy; a decrease in the frequency and/or severity of pain associated with vaginal irritation; a decrease in the frequency and/or severity of symptoms and/or pain associated with allodynia; a decrease in the frequency and/or severity of hypersensitivity of bladder afferent pathways in the absence of bladder pathology; a decrease in the frequency and/or severity of diverticulitis pain; a decrease in the frequency and/or severity of pain associated with gastrointestinal disorders; a decrease in the frequency and/or severity of pain associated with venereal diseases; a decrease in the frequency and/or severity of pain associated with irritable bowel syndrome (IBS); a decrease in the frequency and/or severity of rectal pain; a decrease in the frequency and/or severity of chronic proctalgia; a decrease in the frequency and/or severity of proctalgia fugax; a decrease in the frequency and/or severity of anal pain; a decrease in the frequency and/or severity of chronic anal fissure; a decrease in the frequency and/or severity of post-operative anal pain; a decrease in the frequency and/or severity of pain associated with cancer; a decrease in the frequency and/or severity of pain associated with gastrointestinal tract neoplasms; a decrease in the frequency and/or severity of general pelvic pain; a decrease in the frequency and/or severity of symptoms and/or pain associated with orchialgia; a decrease in the frequency and/or severity of chronic prostatitis; a decrease in the frequency and/or severity of prostatodynia; a decrease in the frequency and/or severity of vulvodynia; a decrease in the frequency and/or severity of urethral syndrome; a decrease in the frequency and/or severity of penile pain; a decrease in the frequency and/or severity of perianal pain; a decrease in the frequency and/or severity of pain associated with ulcerative colitis; a decrease in the frequency and/or severity of ulcerative proctitis; a decrease in the frequency and/or severity of symptoms and/or pain associated with Crohn's disease; or any combination thereof; relative to the number, frequency, and/or severity of these foregoing symptoms and/or pains in the subject prior to being administered the therapeutically effective amount of a peptide of the present disclosure and/or a pharmaceutical composition of the present disclosure.

In some embodiments, limiting the symptoms of, reducing the severity of, or treating a visceral pain condition, includes any measurable decrease or complete inhibition to achieve a desired result. For example, there may be a decrease of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, in the number of symptoms, severity of symptoms, and/or frequency of symptoms (e.g., number, severity, and/or frequency of symptoms and/or pain associated with a visceral pain condition). About as used herein means within ±10%, preferably ±5% of a given value.

Thus, in some embodiments, the terms “limiting the symptoms of,” or “reducing the severity of,” or “treating a visceral pain condition,” refers to: a decrease or reduction in the frequency and/or severity of a symptom and/or pain associated with a visceral pain condition, when a therapeutically effective amount of a peptide of the present disclosure and/or a pharmaceutical composition of the present disclosure are administered to a subject in need thereof, that is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 2.25%, at least about 2.5%, at least about 2.75%, at least about 3%, at least about 3.25%, at least about 3.5%, at least about 3.75%, at least about 4%, at least about 4.25%, at least about 4.5%, at least about 4.75%, at least about 5%, at least about 5.25%, at least about 5.5%, at least about 5.75%, at least about 6%, at least about 6.25%, at least about 6.5%, at least about 6.75%, at least about 7%, at least about 7.25%, at least about 7.5%, at least about 7.75%, at least about 8%, at least about 8.25%, at least about 8.5%, at least about 8.75%, at least about 9%, at least about 9.25%, at least about 9.5%, at least about 9.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, relative to the frequency and/or severity of a symptom and/or a pain associated with a visceral pain condition in the subject prior to receiving the therapeutically effective amount of a peptide of the present disclosure and/or a pharmaceutical composition of the present disclosure.

### Illustrative Embodiments

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

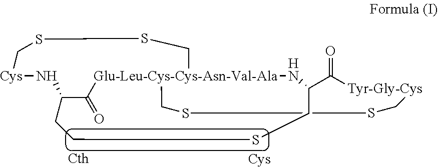

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, a pharmaceutical composition comprises a peptide of the present disclosure, wherein the peptide is in an amount ranging from about 0.0003% w/w to about 0.5% w/w, of the total weight of the pharmaceutical composition.

In some embodiments, a pharmaceutical composition comprises a peptide of the present disclosure, wherein the peptide is in an amount ranging from about 0.000498% w/w to about 0.335% w/w, of the total weight of the pharmaceutical composition.

In some embodiments, a pharmaceutical composition comprises a peptide of the present disclosure, wherein the peptide is in an amount that is about 0.000498% w/w of the total weight of the pharmaceutical composition.

In some embodiments, a pharmaceutical composition comprises a peptide of the present disclosure, wherein the peptide is in an amount that is about 0.00149% w/w of the total weight of the pharmaceutical composition.

In some embodiments, a pharmaceutical composition comprises a peptide of the present disclosure, wherein the peptide is in an amount that is about 0.00299% w/w of the total weight of the pharmaceutical composition.

In some embodiments, a pharmaceutical composition comprises a peptide of the present disclosure, wherein the peptide is in an amount that is about 0.00448% w/w of the total weight of the pharmaceutical composition.

In some embodiments, a pharmaceutical composition comprises a peptide of the present disclosure, wherein the peptide is in an amount that is about 0.00870% w/w of the total weight of the pharmaceutical composition.

In some embodiments, a pharmaceutical composition comprises a peptide of the present disclosure, wherein the peptide is in an amount that is about 0.00896% w/w of the total weight of the pharmaceutical composition.

In some embodiments, a pharmaceutical composition comprises a peptide of the present disclosure, wherein the peptide is in an amount that is about 0.00961% w/w of the total weight of the pharmaceutical composition.

In some embodiments, a pharmaceutical composition comprises a peptide of the present disclosure, wherein the peptide is in an amount that is about 0.0124% w/w of the total weight of the pharmaceutical composition.

In some embodiments, a pharmaceutical composition comprises a peptide of the present disclosure, wherein the peptide is in an amount that is about 0.0261% w/w of the total weight of the pharmaceutical composition.

In some embodiments, a pharmaceutical composition comprises a peptide of the present disclosure, wherein the peptide is in an amount that is about 0.0288% w/w of the total weight of the pharmaceutical composition.

In some embodiments, a pharmaceutical composition comprises a peptide of the present disclosure, wherein the peptide is in an amount that is about 0.301% w/w of the total weight of the pharmaceutical composition.

In some embodiments, a pharmaceutical composition comprises a peptide of the present disclosure, wherein the peptide is in an amount that is about 0.335% w/w of the total weight of the pharmaceutical composition.

In some embodiments, a pharmaceutical composition comprises a peptide of the present disclosure, and an excipient, wherein the excipient is a sodium phosphate buffer.

In some embodiments, the sodium phosphate buffer has a concentration of about 20 mM, and a pH of about 7.0.

In some embodiments, the pharmaceutical composition is formulated in an enteral form; a parenteral form; or a transmucosal form.

In some embodiments, the pharmaceutical composition is formulated in a suppository form, an enema form, a feeding tube form, or a solution for intraluminal use.

In some embodiments, the pharmaceutical composition is formulated as an enema, a rectal gel, a rectal foam, a rectal aerosol, or a suppository.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; and wherein the peptide has an N-terminus that is acetylated.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide is in an amount ranging from about 0.000498% w/w to about 0.0124% w/w, of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein the peptide is in an amount ranging from about 0.000498% w/w to about 0.0124% w/w, of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount ranging from about 0.000498% w/w to about 0.0124% w/w, of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount ranging from about 0.000498% w/w to about 0.0124% w/w, of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.000498% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.000498% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00149% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00149% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00299% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00299% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00448% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00448% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00896% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00896% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.0124% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.0124% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00870% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00961% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.0261% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.0288% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.301% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.335% w/w of the total weight of the pharmaceutical composition; wherein the excipient comprises a sodium phosphate buffer; wherein the pharmaceutical composition has a pH ranging from about 6.9 to about 7.2; and wherein the pharmaceutical composition is formulated as an enema or a rectal foam.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide is in an amount ranging from about 0.000498% w/w to about 0.335% w/w, of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated; wherein the peptide is in an amount ranging from about 0.000498% w/w to about 0.335% w/w, of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount ranging from about 0.000498% w/w to about 0.0124% w/w, of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount ranging from about 0.000498% w/w to about 0.0124% w/w, of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.000498% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.000498% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00149% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00149% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00299% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00299% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00448% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00448% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.00896% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.00896% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is an amount that is about 0.0124% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is an amount that is about 0.0124% w/w of the total weight of the pharmaceutical composition; wherein the excipient comprises a 20 mM sodium phosphate buffer; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

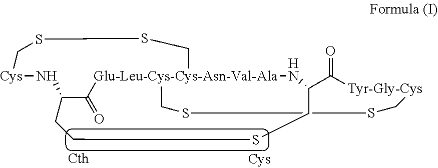

wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, a pharmaceutical composition comprises a peptide of the present disclosure, or a pharmaceutically acceptable salt thereof; and one or more excipients; wherein the peptide is in an amount ranging from about 0.0003% w/w to about 0.5% w/w, of the total weight of the composition.

In some embodiments, a pharmaceutical composition comprises a peptide of the present disclosure, or a pharmaceutically acceptable salt thereof; and one or more excipients; wherein the peptide is in an amount ranging from about 0.0004% w/w to about 0.4% w/w, of the total weight of the composition.

In some embodiments, a pharmaceutical composition comprises a peptide of the present disclosure, or a pharmaceutically acceptable salt thereof; and one or more excipients; wherein the peptide is in an amount ranging from about 0.000498% w/w to about 0.335% w/w, of the total weight of the composition.

In some embodiments, a pharmaceutical composition comprises a peptide of the present disclosure, or a pharmaceutically acceptable salt thereof; and one or more excipients; wherein the peptide is in an amount that is about 0.000498% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.00149% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.00299% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.00448% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.00870% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.00896% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.00961% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.0124% w/w of the total weight of the composition.

In some embodiments, a pharmaceutical composition comprises a peptide of the present disclosure, or a pharmaceutically acceptable salt thereof; and one or more excipients;

wherein the peptide is in an amount that is about 0.0261% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.0288% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.301% w/w of the total weight of the composition.

In some embodiments, a pharmaceutical composition comprises a peptide of the present disclosure, or a pharmaceutically acceptable salt thereof; and one or more excipients; wherein the peptide is in an amount that is about 0.335% w/w of the total weight of the composition.

In some embodiments, a pharmaceutical composition comprises a peptide of the present disclosure, or a pharmaceutically acceptable salt thereof; and one or more excipients; wherein the one or more excipients is one or more buffer salts; solvents; or pH modifiers.

In some embodiments, the one or more buffer salts is a sodium phosphate monobasic monohydrate, and a sodium phosphate dibasic heptahydrate.

In some embodiments, the sodium phosphate monobasic monohydrate in an amount ranging from about 0.05% to about 0.2% w/w, and the sodium phosphate dibasic heptahydrate in an amount ranging from about 0.2% to about 0.4% w/w, of the total weight of the composition.

In some embodiments, the sodium phosphate monobasic monohydrate in an amount that is about 0.116% w/w, and the sodium phosphate dibasic heptahydrate in an amount that is about 0.308% w/w, of the total weight of the composition.

In some embodiments, the one or more solvents is water.

In some embodiments, the water in an amount ranging from about 98% to about 99.8% w/w of the total weight of the composition.

In some embodiments, the water in an amount that is about 99.6% w/w of the total weight of the composition.

In some embodiments, the one or more pH modifiers is a sodium hydroxide or a phosphoric acid.

In some embodiments, the sodium hydroxide or phosphoric acid have a concentration of about 1N.

In some embodiments, the sodium hydroxide or phosphoric acid are added to the pharmaceutical composition to adjust the pH of the pharmaceutical composition to about 6 to about 8.

In some embodiments, the sodium hydroxide or phosphoric acid are added to the pharmaceutical composition to adjust the pH of the pharmaceutical composition to about 6.8 to about 7.2.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; and wherein the peptide has an N-terminus that is optionally acetylated.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; and wherein the peptide has an N-terminus that is optionally acetylated.

In some embodiments, the pharmaceutical composition is formulated as an enteral form; a parenteral form; or a transmucosal form.

In some embodiments, the pharmaceutical composition is formulated as a suppository form, an enema form, a feeding tube form, or a solution for intraluminal use.

In some embodiments, the pharmaceutical composition is formulated as an enema, a rectal gel, a rectal foam, a rectal aerosol, or a suppository.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide is in an amount ranging from about 0.000498% to about 0.335% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide is in an amount ranging from about 0.000498% to about 0.335% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.000498% to about 0.0124% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is in an amount ranging from about 0.000498% to about 0.0124% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.000498% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is in an amount that is about 0.000498% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00149% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is in an amount that is about 0.00149% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00299% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is in an amount that is about 0.00299% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00448% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is in an amount that is about 0.00448% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00896% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is in an amount that is about 0.00896% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0124% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the peptide is in an amount that is about 0.0124% w/w of the of the total weight of the composition; and wherein the one or more excipients comprise: sodium phosphate monobasic monohydrate in an amount that is about 0.116% w/w; sodium phosphate dibasic heptahydrate in an amount that is about 0.308% w/w; and water in an amount that is about 99.6% w/w; of the total weight of the composition; wherein the pharmaceutical composition has a pH ranging from about 6.9 to about 7.2; and wherein the pharmaceutical composition is formulated as an enema.

In some embodiments, present disclosure provides a liquid pharmaceutical composition comprising a peptide, or a pharmaceutically acceptable salt thereof; and a plurality of excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

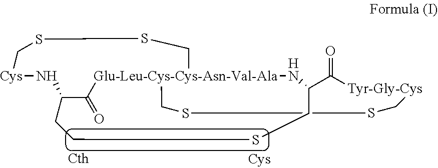

wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is optionally acetylated; wherein the plurality of excipients comprise: 1.165 mg/mL of sodium phosphate monobasic monohydrate; 3.095 mg/mL of sodium phosphate dibasic heptahydrate; and an amount of water resulting in a total volume of the liquid pharmaceutical composition that is 20 mL; wherein the liquid pharmaceutical composition comprises an amount of peptide ranging from about 5 μg/mL to about 125 μg/mL; and wherein the liquid composition has a pH ranging from about 6.9 to about 7.2.

In some embodiments, present disclosure provides a liquid pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is optionally acetylated; wherein the plurality of excipients comprise: 1.165 mg/mL of sodium phosphate monobasic monohydrate; 3.095 mg/mL of sodium phosphate dibasic heptahydrate; and an amount of water resulting in a total volume of the liquid pharmaceutical composition that is 20 mL; wherein the liquid pharmaceutical composition comprises an amount of peptide ranging from about 5 μg/mL to about 125 μg/mL; and wherein the liquid composition has a pH ranging from about 6.9 to about 7.2.

In some embodiments, a liquid pharmaceutical composition comprises an amount of peptide that is about 5.00 μg/mL.

In some embodiments, a liquid pharmaceutical composition comprises an amount of peptide that is about 15.0 μg/mL.

In some embodiments, a liquid pharmaceutical composition comprises an amount of peptide that is about 30.0 μg/mL.

In some embodiments, a liquid pharmaceutical composition comprises an amount of peptide that is about 45.0 μg/mL.

In some embodiments, a liquid pharmaceutical composition comprises an amount of peptide that is about 90.0 μg/mL.

In some embodiments, a liquid pharmaceutical composition comprises an amount of peptide that is about 125.0 μg/mL.

In some embodiments, a liquid pharmaceutical composition comprises an amount of peptide that is about 200.0 μg/mL.

In some embodiments, a liquid pharmaceutical composition comprises an amount of peptide that is about 250.0 μg/mL.

In some embodiments, a liquid pharmaceutical composition comprises an amount of peptide that is about 300.0 g/mL.

In some embodiments, a liquid pharmaceutical composition comprises an amount of peptide that is about 350.0 g/mL.

In some embodiments, a liquid pharmaceutical composition comprises an amount of peptide that is about 400.0 μg/mL.

In some embodiments, a liquid pharmaceutical composition comprises an amount of peptide that is about 450.0 μg/mL.

In some embodiments, a liquid pharmaceutical composition comprises an amount of peptide that is about 500.0 μg/mL.

In some embodiments, a liquid pharmaceutical composition comprises an amount of peptide that is about 550.0 g/mL.

In some embodiments, a liquid pharmaceutical composition comprises an amount of peptide that is about 600.0 μg/mL.

In some embodiments, a liquid pharmaceutical composition comprises an amount of peptide that is about 650.0 g/mL.

In some embodiments, a liquid pharmaceutical composition comprises an amount of peptide that is about 700.0 μg/mL.

In some embodiments, a liquid pharmaceutical composition comprises an amount of peptide that is about 750.0 μg/mL.

In some embodiments, a liquid pharmaceutical composition comprises an amount of peptide that is about 800.0 μg/mL.

In some embodiments, a liquid pharmaceutical composition comprises an amount of peptide that is about 850.0 g/mL.

In some embodiments, a liquid pharmaceutical composition comprises an amount of peptide that is about 900.0 μg/mL.

In some embodiments, a liquid pharmaceutical composition comprises an amount of peptide that is about 950.0 μg/mL.

In some embodiments, a liquid pharmaceutical composition comprises an amount of peptide that is about 1000.0 μg/mL.

In some embodiments, present disclosure provides a liquid pharmaceutical composition comprising a peptide, or a pharmaceutically acceptable salt thereof; and a plurality of excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

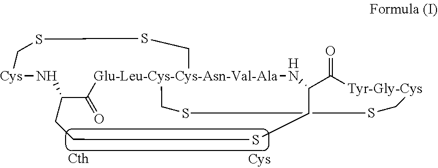

wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is acetylated; wherein the plurality of excipients comprise: 1.165 mg/mL of sodium phosphate monobasic monohydrate; 3.095 mg/mL of sodium phosphate dibasic heptahydrate; and an amount of water resulting in a total volume of the liquid pharmaceutical composition that is 20 mL; wherein the liquid pharmaceutical composition comprises an amount of peptide ranging from about 5 μg/mL to about 125 μg/mL; and wherein the liquid composition has a pH ranging from about 6.9 to about 7.2.

In some embodiments, present disclosure provides a liquid pharmaceutical composition comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is acetylated; wherein the plurality of excipients comprise: 1.165 mg/mL of sodium phosphate monobasic monohydrate; 3.095 mg/mL of sodium phosphate dibasic heptahydrate; and an amount of water resulting in a total volume of the liquid pharmaceutical composition that is 20 mL; wherein the liquid pharmaceutical composition comprises an amount of peptide ranging from about 5 g/mL to about 125 μg/mL; and wherein the liquid composition has a pH ranging from about 6.9 to about 7.2.

In some embodiments, present disclosure provides a unit dosage form comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

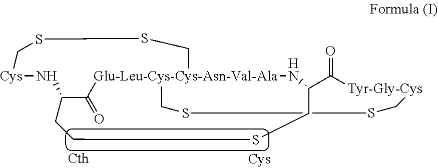

wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is optionally acetylated; and wherein said unit dosage form comprises an amount of the peptide ranging from about 50 μg to about 3000 μg.

In some embodiments, present disclosure provides a unit dosage form comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is optionally acetylated; and wherein said unit dosage form comprises an amount of the peptide ranging from about 50 μg to about 3000 μg.

In some embodiments, the unit dosage form comprises an amount of peptide that is about 50 μg.

In some embodiments, the unit dosage form comprises an amount of peptide that is about 100 μg.

In some embodiments, the unit dosage form comprises an amount of peptide that is about 300 μg.

In some embodiments, the unit dosage form comprises an amount of peptide that is about 600 μg.

In some embodiments, the unit dosage form comprises an amount of peptide that is about 900 μg.

In some embodiments, the unit dosage form comprises an amount of peptide that is about 1800 μg.

In some embodiments, the unit dosage form comprises an amount of peptide that is about 2140 μg.

In some embodiments, the unit dosage form comprises an amount of peptide that is about 2500 μg.

In some embodiments, the unit dosage form comprises an amount of peptide that is about 3000 μg.

In some embodiments, present disclosure provides a unit dosage form comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is optionally acetylated; and wherein said unit dosage form comprises an amount of the peptide ranging from about 50 μg to about 3000 μg; wherein the one or more excipients is a sodium phosphate buffer.

In some embodiments, present disclosure provides a unit dosage form comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is optionally acetylated; and wherein said unit dosage form comprises an amount of the peptide ranging from about 50 μg to about 3000 μg; wherein the one or more excipients is a sodium phosphate buffer.

In some embodiments, present disclosure provides a unit dosage form comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is optionally acetylated; and wherein said unit dosage form comprises an amount of the peptide ranging from about 50 μg to about 3000 μg; wherein the one or more excipients is one or more buffer salts; solvents; or pH modifiers.

In some embodiments, present disclosure provides a unit dosage form comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is optionally acetylated; and wherein said unit dosage form comprises an amount of the peptide ranging from about 50 μg to about 3000 μg; wherein the one or more excipients is one or more buffer salts; solvents; or pH modifiers.

In some embodiments, the unit dosage form comprises one or more excipients, wherein the one or more excipients is one or more buffer salts is a sodium phosphate monobasic monohydrate, and a sodium phosphate dibasic heptahydrate.

In some embodiments, the sodium phosphate monobasic monohydrate in an amount ranging from about 0.05% to about 0.2% w/w, and the sodium phosphate dibasic heptahydrate in an amount ranging from about 0.2% to about 0.4% w/w, of the total weight of the composition.

In some embodiments, the sodium phosphate monobasic monohydrate in an amount that is about 0.116% w/w, and the sodium phosphate dibasic heptahydrate in an amount that is about 0.308% w/w, of the total weight of the composition.

In some embodiments, the unit dosage form comprises one or more excipients, wherein the one or more excipients is one or more solvents.

In some embodiments, the one or more solvents is water.

In some embodiments, the water can be in an amount ranging from about 98% to about 99.8% w/w of the total weight of the composition.

In some embodiments, the water can be in an amount that is about 99.6% w/w of the total weight of the composition.

In some embodiments, the unit dosage form comprises one or more excipients, wherein the one or more excipients can be one or more pH modifiers.

In some embodiments, the unit dosage form comprises one or more excipients, wherein the one or more excipients can be one or more pH modifiers such as a sodium hydroxide or a phosphoric acid.

In some embodiments, the sodium hydroxide or phosphoric acid have a concentration of about 1N.

In some embodiments, the sodium hydroxide or phosphoric acid are added to the pharmaceutical composition to adjust the pH of the pharmaceutical composition to about 6 to about 8.

In some embodiments, the sodium hydroxide or phosphoric acid are added to the pharmaceutical composition to adjust the pH of the pharmaceutical composition to about 6.8 to about 7.2.

In some embodiments, present disclosure provides a unit dosage form comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I); wherein Cth is a cystathionine; wherein the cysteines at positions 1 and 6 (Cys₁ and Cys₆), and positions 5 and 13 (Cys₅ and Cys₁₃) are connected by disulfide bonds; wherein cystathionine at position 2 (Cth₂) and the cysteine at position 10 (Cys₁₀) are connected by a thioether bond; wherein the peptide has an N-terminus that is optionally acetylated; and wherein said unit dosage form comprises an amount of the peptide ranging from about 50 μg to about 3000 μg; wherein the unit dosage form is formulated in an enteral form; a parenteral form; or a transmucosal form.

In some embodiments, present disclosure provides a unit dosage form comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is optionally acetylated; and wherein said unit dosage form comprises an amount of the peptide ranging from about 50 μg to about 3000 μg; wherein the unit dosage form is formulated in an enteral form; a parenteral form; or a transmucosal form.

In some embodiments, the unit dosage form is formulated in a suppository form, an enema form, a feeding tube form, or a solution for intraluminal use.

In some embodiments, the unit dosage form is formulated as an enema, a rectal gel, a rectal foam, a rectal aerosol, or a suppository. In some embodiments, the unit dosage form is formulated as a rectal foam.

In some embodiments, present disclosure provides an enema kit comprising a pharmaceutical composition, a liquid pharmaceutical composition, or a unit dosage form, comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

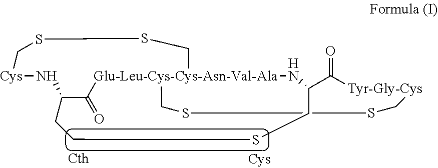

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is optionally acetylated; a syringe, and an enema applicator.

In some embodiments, present disclosure provides an enema kit comprising a pharmaceutical composition, a liquid pharmaceutical composition, or a unit dosage form, comprising a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein Cys₁ and Cys₆, and Cys₅ and Cys₁₃ are connected by disulfide bonds; and wherein Cth₂ and Cys₁₀ are connected by a thioether bond; wherein the peptide has an N-terminus that is optionally acetylated; a syringe, and an enema applicator.

In some embodiments, the syringe is a 50 ml syringe.

In some embodiments, enema applicator is a 15 cm×4.6667 mm enema applicator.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

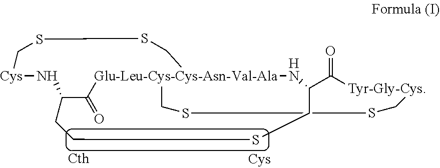

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

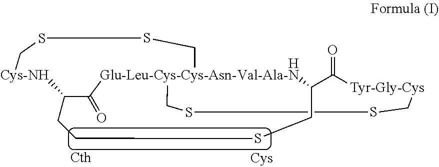

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

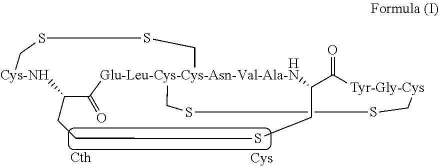

wherein the N-terminus is acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the N-terminus is acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the pharmaceutical composition is formulated as a rectal foam.

In some embodiments, the pharmaceutical composition formulated as a rectal foam comprises a peptide of the present disclosure in an amount ranging from about 0.0003% w/w to about 0.5% w/w, of the total weight of the composition.

In some embodiments, the pharmaceutical composition formulated as a rectal foam comprises a peptide of the present disclosure in an amount ranging from about 0.0004% w/w to about 0.4% w/w, of the total weight of the pharmaceutical composition.

In some embodiments, the pharmaceutical composition formulated as a rectal foam comprises a peptide of the present disclosure in an amount ranging from about 0.000498% w/w to about 0.335% w/w, of the total weight of the pharmaceutical composition.

In some embodiments, the pharmaceutical composition formulated as a rectal foam comprises a peptide of the present disclosure in an amount that is about 0.000498% w/w of the total weight of the pharmaceutical composition.

In some embodiments, the pharmaceutical composition formulated as a rectal foam comprises a peptide of the present disclosure in an amount that is about 0.00149% w/w of the total weight of the pharmaceutical composition.

In some embodiments, the pharmaceutical composition formulated as a rectal foam comprises a peptide of the present disclosure in an amount that is about 0.00299% w/w of the total weight of the pharmaceutical composition.

In some embodiments, the pharmaceutical composition formulated as a rectal foam comprises a peptide of the present disclosure in an amount that is about 0.00448% w/w of the total weight of the pharmaceutical composition.

In some embodiments, the pharmaceutical composition formulated as a rectal foam comprises a peptide of the present disclosure in an amount that is about 0.00870% w/w of the total weight of the pharmaceutical composition.

In some embodiments, the pharmaceutical composition formulated as a rectal foam comprises a peptide of the present disclosure in an amount that is about 0.00896% w/w of the total weight of the pharmaceutical composition.

In some embodiments, the pharmaceutical composition formulated as a rectal foam comprises a peptide of the present disclosure in an amount that is about 0.00961% w/w of the total weight of the pharmaceutical composition.

In some embodiments, the pharmaceutical composition formulated as a rectal foam comprises a peptide of the present disclosure in an amount that is about 0.0124% w/w of the total weight of the pharmaceutical composition.

In some embodiments, the pharmaceutical composition formulated as a rectal foam comprises a peptide of the present disclosure in an amount that is about 0.0261% w/w of the total weight of the pharmaceutical composition.

In some embodiments, the pharmaceutical composition formulated as a rectal foam comprises a peptide of the present disclosure in an amount that is about 0.0288% w/w of the total weight of the pharmaceutical composition.

In some embodiments, the pharmaceutical composition formulated as a rectal foam comprises a peptide of the present disclosure in an amount that is about 0.301% w/w of the total weight of the pharmaceutical composition.

In some embodiments, the pharmaceutical composition formulated as a rectal foam comprises a peptide of the present disclosure in an amount that is about 0.335% w/w of the total weight of the pharmaceutical composition.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

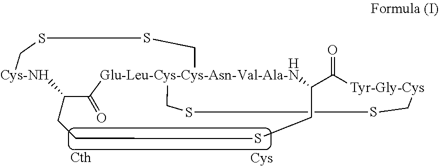

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 50% to about 90%, propylene glycol in an amount ranging from about 5% to about 20%, sodium phosphate dibasic in an amount ranging from about 0.08% to about 0.25%, sodium dihydrogen phosphate monohydrate in an amount ranging from about 0.05% to about 0.20%, disodium EDTA in an amount ranging from about 0.02% to about 0.10%, methylparaben in an amount ranging from about 0.05% to about 0.2%, light mineral oil in an amount ranging from about 3% to about 12%, isopropyl myristate in an amount ranging from about 0.2% to about 1%, white petrolatum in an amount ranging from about 0.5% to about 2%, polyoxyl 20 cetostearyl ether in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.05%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 50% to about 90%, propylene glycol in an amount ranging from about 5% to about 20%, sodium phosphate dibasic in an amount ranging from about 0.08% to about 0.25%, sodium dihydrogen phosphate monohydrate in an amount ranging from about 0.05% to about 0.20%, disodium EDTA in an amount ranging from about 0.02% to about 0.10%, methylparaben in an amount ranging from about 0.05% to about 0.2%, light mineral oil in an amount ranging from about 3% to about 12%, isopropyl myristate in an amount ranging from about 0.2% to about 1%, white petrolatum in an amount ranging from about 0.5% to about 2%, polyoxyl 20 cetostearyl ether in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.05%, w/w % of the total composition, and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

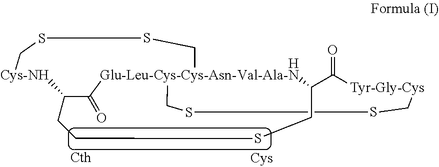

wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 50% to about 90%, propylene glycol in an amount ranging from about 5% to about 20%, sodium phosphate dibasic in an amount ranging from about 0.08% to about 0.25%, sodium dihydrogen phosphate monohydrate in an amount ranging from about 0.05% to about 0.20%, disodium EDTA in an amount ranging from about 0.02% to about 0.10%, methylparaben in an amount ranging from about 0.05% to about 0.2%, light mineral oil in an amount ranging from about 3% to about 12%, isopropyl myristate in an amount ranging from about 0.2% to about 1%, white petrolatum in an amount ranging from about 0.5% to about 2%, polyoxyl 20 cetostearyl ether in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.05%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 50% to about 90%, propylene glycol in an amount ranging from about 5% to about 20%, sodium phosphate dibasic in an amount ranging from about 0.08% to about 0.25%, sodium dihydrogen phosphate monohydrate in an amount ranging from about 0.05% to about 0.20%, disodium EDTA in an amount ranging from about 0.02% to about 0.10%, methylparaben in an amount ranging from about 0.05% to about 0.2%, light mineral oil in an amount ranging from about 3% to about 12%, isopropyl myristate in an amount ranging from about 0.2% to about 1%, white petrolatum in an amount ranging from about 0.5% to about 2%, polyoxyl 20 cetostearyl ether in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.05%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

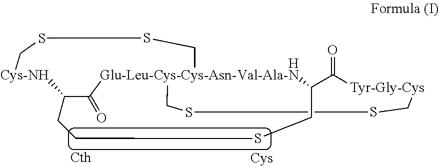

wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 60% to about 80%, propylene glycol in an amount ranging from about 7% to about 15%, sodium phosphate dibasic in an amount ranging from about 0.1% to about 0.18%, sodium dihydrogen phosphate monohydrate in an amount ranging from about 0.07% to about 0.15%, disodium EDTA in an amount ranging from about 0.04% to about 0.6%, methylparaben in an amount ranging from about 0.08% to about 0.15%, light mineral oil in an amount ranging from about 4% to about 8%, isopropyl myristate in an amount ranging from about 0.4% to about 0.8%, white petrolatum in an amount ranging from about 0.8% to about 1.5%, polyoxyl 20 cetostearyl ether in an amount ranging from about 1.5% to about 3%, cetyl alcohol in an amount ranging from about 0.8% to about 1.5%, stearyl alcohol in an amount ranging from about 0.8% to about 1.5%, and propylparaben in an amount ranging from about 0.015% to about 0.03%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 60% to about 80%, propylene glycol in an amount ranging from about 7% to about 15%, sodium phosphate dibasic in an amount ranging from about 0.1% to about 0.18%, sodium dihydrogen phosphate monohydrate in an amount ranging from about 0.07% to about 0.15%, disodium EDTA in an amount ranging from about 0.04% to about 0.6%, methylparaben in an amount ranging from about 0.08% to about 0.15%, light mineral oil in an amount ranging from about 4% to about 8%, isopropyl myristate in an amount ranging from about 0.4% to about 0.8%, white petrolatum in an amount ranging from about 0.8% to about 1.5%, polyoxyl 20 cetostearyl ether in an amount ranging from about 1.5% to about 3%, cetyl alcohol in an amount ranging from about 0.8% to about 1.5%, stearyl alcohol in an amount ranging from about 0.8% to about 1.5%, and propylparaben in an amount ranging from about 0.015% to about 0.03%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

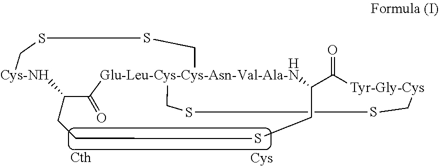

wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 50% to about 90%, propylene glycol in an amount ranging from about 8% to about 30%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, emulsifying wax in an amount ranging from about 0.7% to about 3%, polyoxylene (10) stearyl ether in an amount ranging from about 0.7% to about 3%, cetyl alcohol in an amount ranging from about 0.4% to about 1.4%, and propylparaben in an amount ranging from about 0.01% to about 0.04%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 50% to about 90%, propylene glycol in an amount ranging from about 8% to about 30%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, emulsifying wax in an amount ranging from about 0.7% to about 3%, polyoxylene (10) stearyl ether in an amount ranging from about 0.7% to about 3%, cetyl alcohol in an amount ranging from about 0.4% to about 1.4%, and propylparaben in an amount ranging from about 0.01% to about 0.04%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

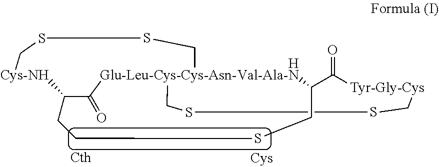

wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 60% to about 85%, propylene glycol in an amount ranging from about 10% to about 20%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, emulsifying wax in an amount ranging from about 1% to about 2%, polyoxylene (10) stearyl ether in an amount ranging from about 1% to about 2%, cetyl alcohol in an amount ranging from about 0.6% to about 0.9%, and propylparaben in an amount ranging from about 0.015% to about 0.03%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 60% to about 85%, propylene glycol in an amount ranging from about 10% to about 20%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, emulsifying wax in an amount ranging from about 1% to about 2%, polyoxylene (10) stearyl ether in an amount ranging from about 1% to about 2%, cetyl alcohol in an amount ranging from about 0.6% to about 0.9%, and propylparaben in an amount ranging from about 0.015% to about 0.03%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

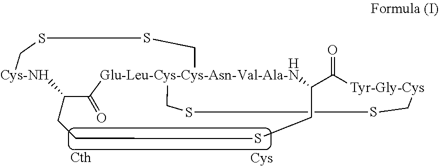

wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 50% to about 90%, propylene glycol in an amount ranging from about 5% to about 20%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, light mineral oil in an amount ranging from about 3% to about 14%, white wax in an amount ranging from about 0.2% to about 1%, mono- and di-glycerides in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.04%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 50% to about 90%, propylene glycol in an amount ranging from about 5% to about 20%, sodium phosphate dibasic in an amount ranging from about 0.08% to about 0.30%, sodium dihydrogen phosphate monohydrate in an amount ranging from about 0.05% to about 0.2%, disodium EDTA in an amount ranging from about 0.02% to about 0.1%, methylparaben in an amount ranging from about 0.05% to about 0.2%, light mineral oil in an amount ranging from about 3% to about 14%, white wax in an amount ranging from about 0.2% to about 1%, mono- and di-glycerides in an amount ranging from about 1% to about 5%, cetyl alcohol in an amount ranging from about 0.5% to about 2%, stearyl alcohol in an amount ranging from about 0.5% to about 2%, and propylparaben in an amount ranging from about 0.01% to about 0.04%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

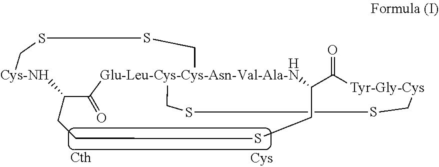

wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 60% to about 80%, propylene glycol in an amount ranging from about 8% to about 15%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, light mineral oil in an amount ranging from about 5% to about 9%, white wax in an amount ranging from about 0.4% to about 0.8%, mono- and di-glycerides in an amount ranging from about 2% to about 3%, cetyl alcohol in an amount ranging from about 0.8% to about 1.5%, stearyl alcohol in an amount ranging from about 0.8% to about 1.5%, and propylparaben in an amount ranging from about 0.015% to about 0.03%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the one or more excipients is purified water in an amount ranging from about 60% to about 80%, propylene glycol in an amount ranging from about 8% to about 15%, sodium phosphate dibasic in an amount ranging from about 0.1% to about 0.2%, sodium dihydrogen phosphate monohydrate in an amount ranging from about 0.08% to about 0.15%, disodium EDTA in an amount ranging from about 0.04% to about 0.08%, methylparaben in an amount ranging from about 0.08% to about 0.15%, light mineral oil in an amount ranging from about 5% to about 9%, white wax in an amount ranging from about 0.4% to about 0.8%, mono- and di-glycerides in an amount ranging from about 2% to about 3%, cetyl alcohol in an amount ranging from about 0.8% to about 1.5%, stearyl alcohol in an amount ranging from about 0.8% to about 1.5%, and propylparaben in an amount ranging from about 0.015% to about 0.03%, w/w % of the total composition; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

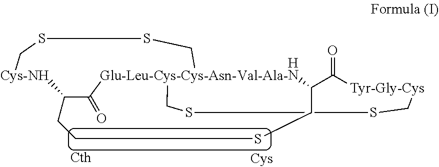

wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the pharmaceutical composition has a pH of about 6 to about 8; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the N-terminus is optionally acetylated; wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the pharmaceutical composition has a pH of about 6 to about 8; and wherein the pharmaceutical composition is formulated as a rectal foam.

In some embodiments, a pharmaceutical composition formulated as a rectal foam has a pH of about 6.8 to about 7.2.

In some embodiments, a pharmaceutical composition formulated as a rectal foam has an N-terminus that is acetylated.

In some embodiments, a pharmaceutical composition formulated as a rectal foam has an N-terminus that is not acetylated.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

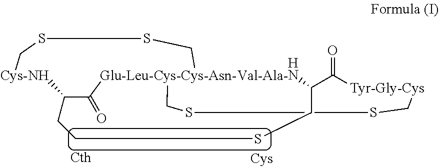

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.000498% to about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.000498% to about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

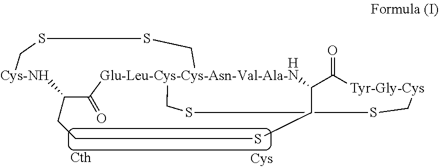

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.000498% to about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.000498% to about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

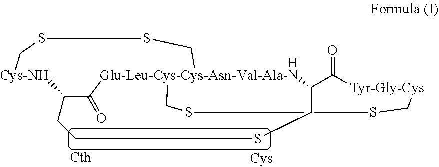

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.000498% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein the peptide is in an amount that is about 0.000498% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

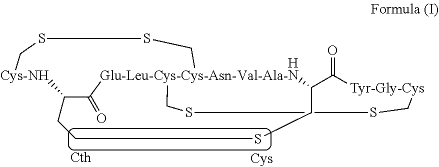

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00149% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00149% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

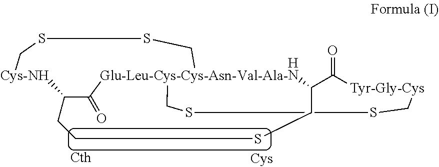

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00299% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00299% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

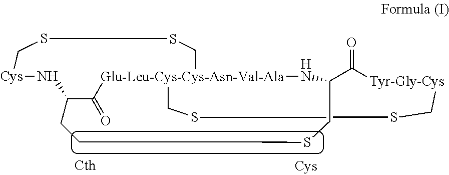

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00448% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00448% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

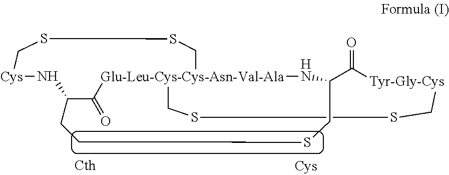

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00870% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00870% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

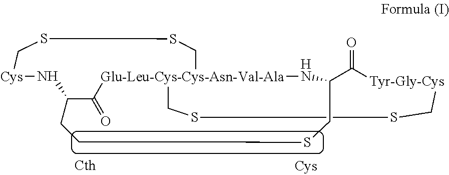

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00896% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00896% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

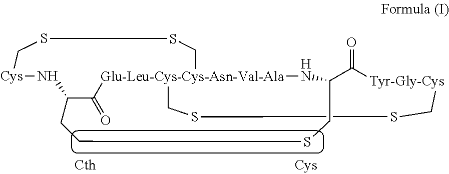

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00961% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.00961% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

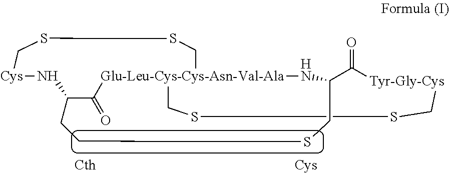

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0124% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0124% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

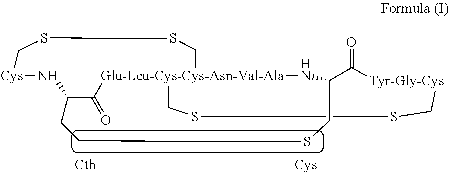

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0261% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0261% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

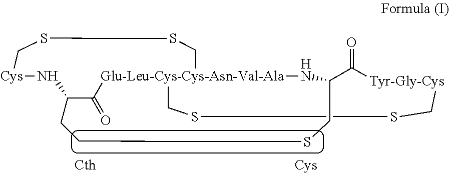

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0288% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0288% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

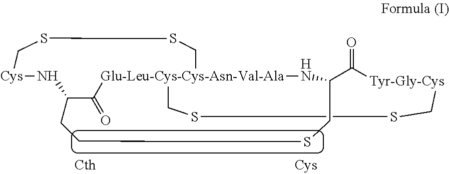

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.301% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.301% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I):

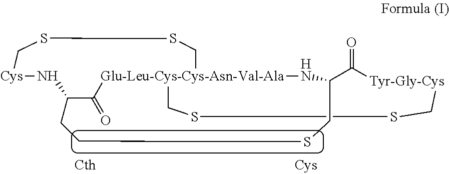

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, a pharmaceutical composition comprises a peptide, or a pharmaceutically acceptable salt thereof; and one or more excipients; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.335% w/w of the of the total weight of the composition; and wherein the one or more excipients comprise: water in an amount that is about 77.5495% w/w; propylene glycol in an amount that is about 10% w/w; sodium phosphate dibasic in an amount that is about 0.1640% w/w; sodium dihydrogen phosphate monohydrate in an amount that is about 0.1165% w/w; disodium EDTA in an amount that is about 0.05% w/w; methylparaben in an amount that is about 0.1% w/w; light mineral oil in an amount that is about 6% w/w; isopropyl myristate in an amount that is about 0.5% w/w; white petrolatum in an amount that is about 1% w/w; polyoxyl 20 cetostearyl ether in an amount that is about 2.5% w/w; cetyl alcohol in an amount that is about 1% w/w; stearyl alcohol in an amount that is about 1% w/w; and propylparaben in an amount that is about 0.02% w/w; of the total weight of the composition; wherein the pharmaceutical composition has a pH ranging from about 6.9 to about 7.2; and wherein the pharmaceutical composition is formulated as a rectal foam.

In some embodiments, present disclosure provides a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

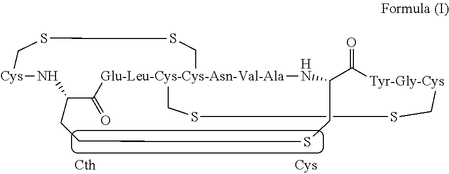

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide is in an amount ranging from about 0.0070% w/w to about 0.0400% w/w, of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0087% w/w to about 0.0260% w/w, of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0087% w/w of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0260% w/w of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; w/w % of the total composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; w/w % of the total composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is DME, A17, A31, AP35, A46, A48, A70, or AP70.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is DME or AP35.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is AP35.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is AP35, and wherein the AP35 in an amount ranging from about 8% to about 10%, w/w % of the total composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is AP35, and wherein the AP35 in an amount ranging from about 9.8835% to about 9.8838%, w/w % of the total composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; and wherein the propellant is AP35 in an amount ranging from about 9.8835% to about 9.8838% w/w %, of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁o Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; and wherein the propellant is AP35 in an amount ranging from about 9.8835% to about 9.8838%, w/w % of the total weight of the pharmaceutical rectal foam composition.

In some embodiments, present disclosure provides a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

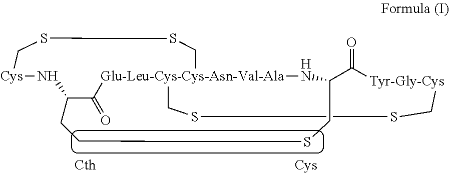

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is acetylated.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount ranging from about 0.0070% w/w to about 0.0400% w/w, of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount ranging from about 0.0080% w/w to about 0.0300% w/w, of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount ranging from about 0.0087% w/w to about 0.0260% w/w, of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount that is about 0.0087% w/w of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount that is about 0.0260% w/w of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; w/w % of the total composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; w/w % of the total composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is DME, A17, A31, AP35, A46, A48, A70, or AP70.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is DME or AP35.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is AP35.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is AP35, and wherein the AP35 in an amount ranging from about 8% to about 10%, w/w % of the total composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is AP35, and wherein the AP35 in an amount ranging from about 9.8835% to about 9.8838%, w/w % of the total composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; and wherein the propellant is AP35 in an amount ranging from about 9.8835% to about 9.8838% w/w %, of the total weight of the pharmaceutical rectal foam composition.

In some embodiments, a pharmaceutical rectal foam composition comprises a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; and wherein the propellant is AP35 in an amount ranging from about 9.8835% to about 9.8838%, w/w % of the total weight of the pharmaceutical rectal foam composition.

In some embodiments, the present disclosure provides a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

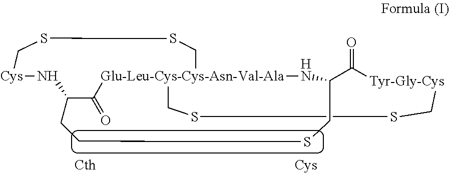

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0087% w/w of the of the total weight of the composition; and wherein the one or more excipients comprise: water in an amount that is about 69.8847% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a pharmaceutical rectal foam composition comprises a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

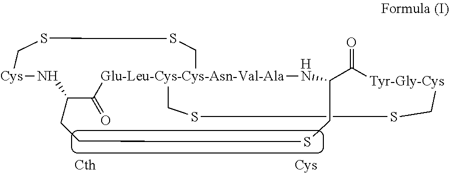

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0260% w/w of the of the total weight of the composition; and wherein the one or more excipients comprise: water in an amount that is about 69.8587% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a pharmaceutical rectal foam composition comprises a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

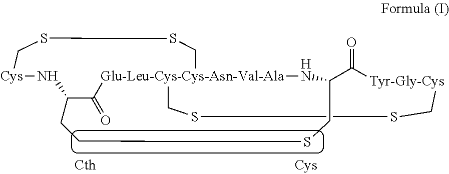

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0087% w/w of the of the total weight of the composition; and wherein the one or more excipients comprise: water in an amount that is about 69.8847% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a pharmaceutical rectal foam composition comprises a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

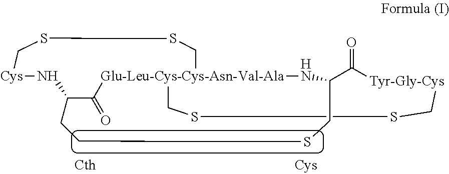

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0260% w/w of the of the total weight of the composition; and wherein the one or more excipients comprise: water in an amount that is about 69.8587% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

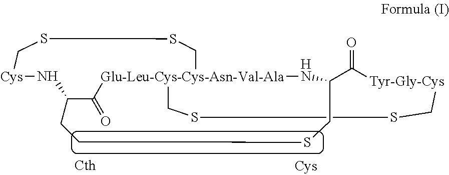

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide is in an amount ranging from about 0.0070% w/w to about 0.0400% w/w, of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0087% w/w to about 0.0260% w/w, of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0087% w/w of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0260% w/w of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; w/w % of the total composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; w/w % of the total composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is DME, A17, A31, AP35, A46, A48, A70, or AP70.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is DME or AP35.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is AP35.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is AP35, and wherein the AP35 in an amount ranging from about 8% to about 10%, w/w % of the total composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; wherein the propellant is AP35, and wherein the AP35 in an amount ranging from about 9.8835% to about 9.8838%, w/w % of the total composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; and wherein the propellant is AP35 in an amount ranging from about 9.8835% to about 9.8838% w/w %, of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount ranging from about 0.0080% w/w to about 0.0300% w/w, of the total weight of the pharmaceutical rectal foam composition; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; and wherein the propellant is AP35 in an amount ranging from about 9.8835% to about 9.8838%, w/w % of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

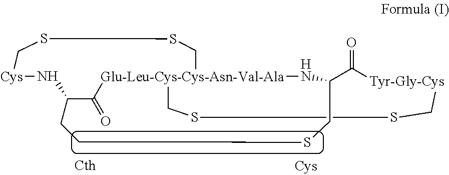

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is acetylated.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount ranging from about 0.0070% w/w to about 0.0400% w/w, of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount ranging from about 0.0080% w/w to about 0.0300% w/w, of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount ranging from about 0.0087% w/w to about 0.0260% w/w, of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount that is about 0.0087% w/w of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; and wherein the peptide is in an amount that is about 0.0260% w/w of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; w/w % of the total composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; w/w % of the total composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is DME, A17, A31, AP35, A46, A48, A70, or AP70.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is DME or AP35.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is AP35.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is AP35, and wherein the AP35 in an amount ranging from about 8% to about 10%, w/w % of the total composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; wherein the propellant is AP35, and wherein the AP35 in an amount ranging from about 9.8835% to about 9.8838%, w/w % of the total composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; and wherein the propellant is AP35 in an amount ranging from about 9.8835% to about 9.8838% w/w %, of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount ranging from about 0.0080% w/w to about 0.0300% w/w, of the total weight of the pharmaceutical rectal foam composition; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; and wherein the propellant is AP35 in an amount ranging from about 9.8835% to about 9.8838%, w/w % of the total weight of the pharmaceutical rectal foam composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

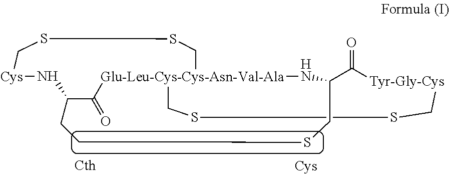

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0087% w/w of the of the total weight of the composition; and wherein the one or more excipients comprise: water in an amount that is about 69.8847% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

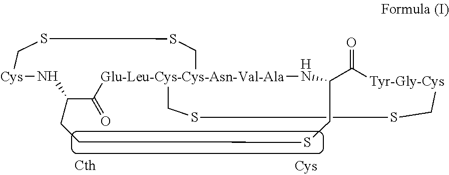

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0260% w/w of the of the total weight of the composition; and wherein the one or more excipients comprise: water in an amount that is about 69.8587% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

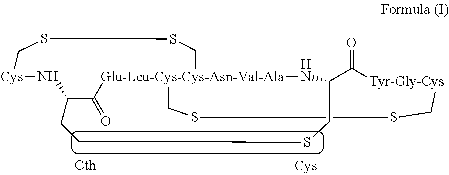

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0087% w/w of the of the total weight of the composition; and wherein the one or more excipients comprise: water in an amount that is about 69.8847% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a canister of the present disclosure comprises a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

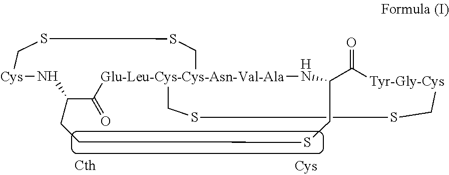

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide has an N-terminus that is acetylated; wherein the peptide is in an amount that is about 0.0260% w/w of the of the total weight of the composition; and wherein the one or more excipients comprise: water in an amount that is about 69.8587% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a kit can comprise any of the peptides of the present disclosure, a pharmaceutical composition of the present disclosure (e.g., a pharmaceutical rectal foam composition), and/or a canister comprising the same. For example, in some embodiments, a kit can comprise a peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃; are connected by disulfide bonds; and wherein Cth₂ and Cys₁₀ are connected by a thioether bond; or a pharmaceutically acceptable salt, hydrate or solvate thereof, or a combination, mixture, or composition comprising the same.

### Illustrative Methods

In some embodiments, the present disclosure provides a method of treating a visceral pain condition in a subject in need thereof, the method comprising: administering to the subject in need thereof a therapeutically effective amount of a peptide, or a pharmaceutically acceptable salt thereof; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical that is at least 80%, at least 85%, at least 90%, or at least 95% identical to an amino acid sequence according to Formula (I):

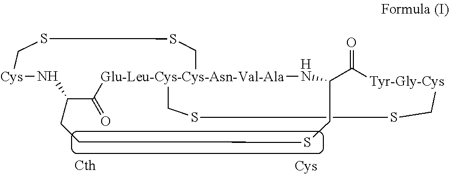

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the N-terminus that is optionally acetylated.

In some embodiments, the present disclosure provides a method of treating a visceral pain condition in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical that is at least 80%, at least 85%, at least 90%, or at least 95% identical to an amino acid sequence according to Formula (I):

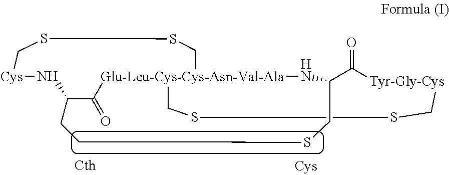

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the N-terminus that is optionally acetylated.

In some embodiments, the present disclosure provides a method of treating a visceral pain condition in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated.

In some embodiments, the present disclosure provides a method of treating a visceral pain condition in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the pharmaceutical composition is formulated in a parenteral form; or a transmucosal form.

In some embodiments, the present disclosure provides a method of treating a visceral pain condition in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the pharmaceutical composition is formulated in a suppository form, an enema form, a feeding tube form, or a solution for intraluminal use.

In some embodiments, the present disclosure provides a method of treating a visceral pain condition in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the pharmaceutical composition is formulated as an enema, a rectal gel, a rectal foam, a rectal aerosol, or a suppository.

In some embodiments, the present disclosure provides a method of treating a visceral pain condition in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the visceral pain condition is selected from: interstitial cystitis/bladder pain syndrome (IC/BPS); bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology; diverticulitis pain; pain associated with gastrointestinal disorders; pain associated with venereal diseases; pain associated with irritable bowel syndrome (IBS); rectal pain; chronic proctalgia; proctalgia fugax; anal pain; chronic anal fissure; post-operative anal pain; pain associated with cancer; pain associated with gastrointestinal tract neoplasms; general pelvic pain; orchialgia; chronic prostatitis; prostatodynia; vulvodynia; urethral syndrome; penile pain; perianal pain; and pain associated with ulcerative colitis; ulcerative proctitis; Crohn's disease; or any combination thereof.

In some embodiments, the present disclosure provides a method of treating a visceral pain condition in a subject in need thereof, the method comprising: administering to the subject in need thereof a therapeutically effective amount of a pharmaceutical composition comprising a peptide, or a pharmaceutically acceptable salt thereof; and an excipient; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the visceral pain condition is interstitial cystitis/bladder pain syndrome (IC/BPS).

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

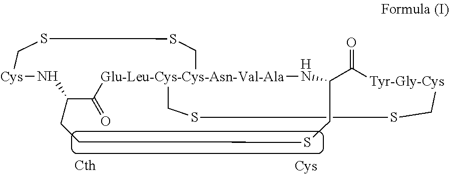

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the peptide is in an amount ranging from about 0.0070% w/w to about 0.0400% w/w, of the total weight of the pharmaceutical rectal foam composition.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the peptide is in an amount ranging from about 0.0080% w/w to about 0.0300% w/w, of the total weight of the pharmaceutical rectal foam composition.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the peptide is in an amount ranging from about 0.0087% w/w to about 0.0260% w/w, of the total weight of the pharmaceutical rectal foam composition.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the peptide is in an amount that is about 0.0087% w/w of the total weight of the pharmaceutical rectal foam composition.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the peptide is in an amount that is about 0.0260% w/w of the total weight of the pharmaceutical rectal foam composition.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the one or more excipients is purified water in an amount ranging from about 60% to about 80%; propylene glycol in an amount ranging from about 8% to about 11%; sodium phosphate dibasic in an amount ranging from about 0.1% to about 0.2%; sodium dihydrogen phosphate monohydrate in an amount ranging from about 0.1% to about 0.2%; disodium EDTA in an amount ranging from about 0.04% to about 0.06%; methylparaben in an amount ranging from about 0.08% to about 0.15%; light mineral oil in an amount ranging from about 4% to about 7%; isopropyl myristate in an amount ranging from about 0.3% to about 0.6%; white petrolatum in an amount ranging from about 0.8% to about 1.5%; polyoxyl 20 cetostearyl ether in an amount ranging from about 2% to about 3%; cetyl alcohol in an amount ranging from about 0.8% to about 1.5%; stearyl alcohol in an amount ranging from about 0.8% to about 1.5%; and propylparaben in an amount ranging from about 0.01% to about 0.03%; w/w % of the total composition.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala Cys₁o Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the one or more excipients is purified water in an amount ranging from about 69.8847% to about 77.5495%; propylene glycol in an amount ranging from about 9.0116% to about 10.0000%; sodium phosphate dibasic in an amount ranging from about 0.1478% to about 0.1640%; sodium dihydrogen phosphate monohydrate in an amount ranging from about 0.1050% to about 0.1165%; disodium EDTA in an amount ranging from about 0.0451% to about 0.0500%; methylparaben in an amount ranging from about 0.0901% to about 0.1000%; light mineral oil in an amount ranging from about 5.4070% to about 6.0000%; isopropyl myristate in an amount ranging from about 0.4506% to about 0.5000%; white petrolatum in an amount ranging from about 0.9012% to about 1.0000%; polyoxyl 20 cetostearyl ether in an amount ranging from about 2.2529% to about 2.5000%; cetyl alcohol in an amount ranging from about 0.9012% to about 1.0000%; stearyl alcohol in an amount ranging from about 0.9012% to about 1.0000%; and propylparaben in an amount ranging from about 0.0180% to about 0.0200%; w/w % of the total composition.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the propellant is DME, A17, A31, AP35, A46, A48, A70, or AP70.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the propellant is DME or AP35.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; and wherein the propellant is AP35.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the propellant is AP35, and wherein the AP35 in an amount ranging from about 8% to about 10%, w/w % of the total composition.

In some embodiments, the present disclosure provides a method of treating a visceral pain condition in a subject in need thereof, the method comprising: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the propellant is AP35, and wherein the AP35 in an amount ranging from about 9.8835% to about 9.8838%, w/w % of the total composition.

In some embodiments, a method of treating a visceral pain condition in a subject in need thereof, comprises: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 80%, at least 85%, at least 90%, or at least 95% identical to an amino acid sequence according to Formula (I):

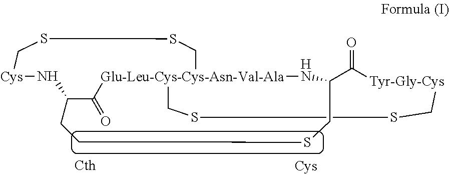

wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; wherein the peptide is in an amount that is about 0.0087% w/w of the of the total weight of the composition; and wherein the one or more excipients comprise: water in an amount that is about 69.8847% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a method of treating a visceral pain condition in a subject in need thereof, comprises: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the peptide is in an amount that is about 0.0260% w/w of the of the total weight of the composition; and wherein the one or more excipients comprise: water in an amount that is about 69.8587% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a method of treating a visceral pain condition in a subject in need thereof, comprises: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the peptide is in an amount that is about 0.0087% w/w of the of the total weight of the composition; and wherein the one or more excipients comprise: water in an amount that is about 69.8847% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a method of treating a visceral pain condition in a subject in need thereof, comprises: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the peptide is in an amount that is about 0.0260% w/w of the of the total weight of the composition; and wherein the one or more excipients comprise: water in an amount that is about 69.8587% w/w; propylene glycol in an amount that is about 9.0116% w/w; sodium phosphate dibasic in an amount that is about 0.1478% w/w; sodium dihydrogen phosphate monohydrate in an amount that is about 0.1050% w/w; disodium EDTA in an amount that is about 0.0451% w/w; methylparaben in an amount that is about 0.0901% w/w; light mineral oil in an amount that is about 5.4070% w/w; isopropyl myristate in an amount that is about 0.4506% w/w; white petrolatum in an amount that is about 0.9012% w/w; polyoxyl 20 cetostearyl ether in an amount that is about 2.2529% w/w; cetyl alcohol in an amount that is about 0.9012% w/w; stearyl alcohol in an amount that is about 0.9012% w/w; propylparaben in an amount that is about 0.0180% w/w; and wherein the propellant is AP35 in an amount that is about 9.8837% w/w; of the total weight of the composition.

In some embodiments, a method of treating a visceral pain condition in a subject in need thereof, comprises: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the visceral pain condition is selected from: interstitial cystitis/bladder pain syndrome (IC/BPS); bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology; diverticulitis pain; pain associated with gastrointestinal disorders; pain associated with venereal diseases; pain associated with irritable bowel syndrome (IBS); rectal pain; chronic proctalgia; proctalgia fugax; anal pain; chronic anal fissure; post-operative anal pain; pain associated with cancer; pain associated with gastrointestinal tract neoplasms; general pelvic pain; orchialgia; chronic prostatitis; prostatodynia; vulvodynia; urethral syndrome; penile pain; perianal pain; and pain associated with ulcerative colitis; ulcerative proctitis; Crohn's disease; or any combination thereof.

In some embodiments, a method of treating a visceral pain condition in a subject in need thereof, comprises: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated; wherein the visceral pain condition is interstitial cystitis/bladder pain syndrome (IC/BPS).

In some embodiments, a method of treating a IC/BPS in a subject in need thereof, comprises: administering to the subject in need thereof a therapeutically effective amount of a pharmaceutical rectal foam composition comprising a peptide, or a pharmaceutically acceptable salt thereof; one or more excipients; and a propellant; wherein the peptide comprises an amino acid sequence that is at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); wherein Cth is a cystathionine; wherein the cysteines at positions 1 and 6 (Cys₁ and Cys₆), and positions 5 and 13 (Cys₅ and Cys₁₃) are connected by disulfide bonds; and wherein cystathionine at position 2 (Cth₂) and the cysteine at position 10 (Cys₁₀) are connected by a thioether bond; and wherein the peptide has an N-terminus that is optionally acetylated.

Any of the peptides of the present disclosure, and/or any of the pharmaceutical compositions, liquid pharmaceutical compositions, unit dosage form, or pharmaceutical rectal foam compositions of the present disclosure, can be used to practice the methods of the present disclosure. For example, a peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃; are connected by disulfide bonds; and wherein Cth₂ and Cys₁₀ are connected by a thioether bond; or a pharmaceutically acceptable salt, hydrate or solvate thereof, or a pharmaceutical composition, liquid pharmaceutical composition, unit dosage form, or pharmaceutical rectal foam composition comprising the same, can be used in the method contemplated by the present disclosure to treat a visceral pain condition in a subject in need thereof, wherein the method comprises: selecting a patient having a visceral pain condition, and administering to the subject a therapeutically effective amount of the peptide comprising the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1), wherein Cth is a cystathionine; wherein Cys₁ and Cys₆; Cys₅ and Cys₁₃; are connected by disulfide bonds; and wherein Cth₂ and Cys₁₀ are connected by a thioether bond; or a pharmaceutically acceptable salt, hydrate or solvate thereof, or a pharmaceutical composition, liquid pharmaceutical composition, unit dosage form, or pharmaceutical rectal foam composition comprising the same.

### EXAMPLES

The following examples are merely illustrative of the present disclosure and should not be construed as limiting the scope of the invention in any way as many variations and equivalents that are encompassed by the present disclosure will become apparent to those skilled in the art upon reading the present disclosure.

### Example 1. General Overview: Description of Pharmaceutical Composition

### Dosage Form

A pharmaceutical composition was prepared comprising a peptide having an amino acid sequence of Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; Cys₅ and Cys₁₃; are connected by disulfide bonds; and wherein Cth₂ and Cys₁₀ are connected by a thioether bond; wherein the peptide has an acetylated N-terminus; and an unmodified C-terminus (hereinafter referred to as “the peptide”); and an excipient.

A unit dosage form of the composition was produced, and was formulated for rectal use (i.e., as an enema). The enema solution created, a medicated, small-volume enema, was a clear solution comprising the peptide of the present disclosure in six concentrations: 5.00 μg/mL, 15.0 μg/mL, 30.0 μg/mL, 45.0 μg/mL, 90.0 μg/mL, and 125 μg/mL; thus, a 20 mL dose volume of each enema solution concentration corresponds to the following unit doses, respectively: 100 μg, 300 μg, 600 μg, 900 μg, 1800 μg, and 2500 μg.

### Components

In addition to the peptide of the present disclosure, the composition of the present disclosure comprises a 20 mM sodium phosphate buffer (pH=7.0) containing water, sodium phosphate monobasic monohydrate, and sodium phosphate dibasic heptahydrate.

During preparation of the buffer, the pH may be adjusted by adding 1N sodium hydroxide or 1N phosphoric acid. The quantitative components of the pharmaceutical composition of the present disclosure are provided in the table below.

TABLE 1
Pharmaceutical composition ingredients. For each concentration of enema, the dose volume is 20 mL. Total
concentration of the peptide for each 20 mL is: 100 μg, 300 μg, 600 μg, 900 μg, 1800 μg, and 2500 μg.

| Component | Composition (% w/w) | | | | | | Quantity (mg/dose) |
|---|---|---|---|---|---|---|---|
| Enema Dose | 100 μg | 300 μg | 600 μg | 900 μg | 1800 μg | 2500 μg | 100 μg |
| Peptide^{a} | 0.000498 | 0.00149 | 0.00299 | 0.00448 | 0.00896 | 0.0124 | 0.100 |
| Sodium | 0.116 | 0.116 | 0.116 | 0.116 | 0.116 | 0.116 | 23.3 |
| Phosphate | | | | | | | |
| Monobasic | | | | | | | |
| Monohydrate | | | | | | | |
| Sodium | 0.308 | 0.308 | 0.308 | 0.308 | 0.308 | 0.308 | 61.9 |
| Phosphate | | | | | | | |
| Dibasic | | | | | | | |
| Heptahydrate | | | | | | | |
| Sterile Water | 99.6 | 99.6 | 99.6 | 99.6 | 99.6 | 99.6 | 20000^{b} |
| for Irrigation | | | | | | | |
| Sodium | N/A^{c} | N/A^{c} | N/A^{c} | N/A^{c} | N/A^{c} | N/A^{c} | N/A^{c} |
| Hydroxide | | | | | | | |
| Phosphoric | N/A^{c} | N/A^{c} | N/A^{c} | N/A^{c} | N/A^{c} | N/A^{c} | N/A^{c} |
| Acid | | | | | | | |
| Total | 100 | 100 | 100 | 100 | 100 | 100 | N/A^{b} |
| (Bulk Enema) | | | | | | | |

| Component | Quantity (mg/dose) | | |
|---|---|---|---|
| Enema Dose | 300 μg | 600 μg | 900 μg |
| Peptide^{a} | 0.300 | 0.600 | 0.900 |
| Sodium | 23.3 | 23.3 | 23.3 |
| Phosphate | | | |
| Monobasic | | | |
| Monohydrate | | | |
| Sodium | 61.9 | 61.9 | 61.9 |
| Phosphate | | | |
| Dibasic | | | |
| Heptahydrate | | | |
| Sterile Water | 20000^{c} | 20000^{d} | 20000^{c} |
| for Irrigation | | | |
| Sodium | N/A^{c} | N/A^{c} | N/A^{c} |
| Hydroxide | | | |
| Phosphoric | N/A^{c} | N/A^{c} | N/A^{c} |
| Acid | | | |
| Total | N/A^{b} | N/A^{bError! Reference source not found.} | N/A^{bError! Reference source not found.} |
| (Bulk Enema) | | | |

| Component | Quantity (mg/dose) | | Quality | |
|---|---|---|---|---|
| Enema Dose | 1800 μg | 2500 μg | Std. | Function |
| Peptide^{a} | 1.80 | 2.50 | In House | Active |
| Sodium | 23.3 | 23.3 | USP | Buffer |
| Phosphate | | | | Salt |
| Monobasic | | | | |
| Monohydrate | | | | |
| Sodium | 61.9 | 61.9 | USP | Buffer |
| Phosphate | | | | Salt |
| Dibasic | | | | |
| Heptahydrate | | | | |
| Sterile Water | 20000^{f} | 20000^{g} | USP | Solvent |
| for Irrigation | | | | |
| Sodium | N/A^{c} | N/A^{c} | NF | pH |
| Hydroxide | | | | Modifier |
| Phosphoric | N/A^{c} | N/A^{c} | NF | pH |
| Acid | | | | Modifier |
| Total | N/A^{b} | N/A^{bError! Reference source not found.} | — | — |
| (Bulk Enema) | | | | |

^{a}= The actual quantity of peptide drug substance is corrected for purity.
^{b}= Dose of 20 mL will be measured by volume. Exact mass of solvent is unknown.
^{c}= Sodium hydroxide and phosphoric acid are used as needed to adjust the buffer pH to 7.0 ± 0.2.

### Type of Container and Closure for Dosage Form

Bulk pharmaceutical compositions of the present disclosure were stored in amber glass bottles with a PTFE-lined screw cap. The proper dose volume was measured and delivered immediately using a 50 mL polypropylene syringe at the time of administration.

### Example 2. Pharmaceutical Development

### Drug Substance

The physicochemical properties of the drug substance are summarized in the table below. Briefly the peptide is a 13 amino acid peptide with a primary amino acid of Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus (hereinafter referred to as “the peptide”).

The peptide is slightly soluble (>1 mg/mL) in water and aqueous buffer at pH 7. The peptide is minimally absorbed and is designed to act locally in the gastrointestinal tract. In addition, the peptide is stable up to 50-months with no appreciable change in purity, or impurity profile when stored under frozen conditions (−20° C.). See Example 20 below.

TABLE 2
General properties of the peptide of the present disclosure.

| Characteristics | Value | |
|---|---|---|
| Appearance | White to off-white powder | |
| Melting point | Between 233.2° C. and 239.6° C. | |
| (decomposition range) | | |
| Stereochemistry: | [α]^{D} = −112.68° (0.01 g/mL | |
| Optical rotation | in H₂O/acetonitrile 9:1 v/v at 25° C.) | |
| Solubility at | Solvent | Solubility |
| ambient temperature | Phosphate buffer (pH 7.0) | >1 mg/mL |
| | Water | >1 mg/mL |
| | Dimethyl sulfoxide | >1 mg/mL |
| | Hydrochloric acid (0.01N) | >1 mg/mL |
| | Methyl tert-butyl ether | Insoluble |
| | Acetonitrile | Insoluble |

| | |
|---|---|
| Dissociation | pKa 1: 3.87 ± 0.01 (pH-metric) |
| constants (pKa) | pKa 2: 4.92 ± 0.01 (pH-metric) |
| | pKa 3: 10.11 ± 0.01 (UV-metric) |
| UV/Visible | Maximum absorbance: 220 nm |
| characteristics | Secondary peak: 278-280 nm |
| Polymorphic form | Amorphous solid |
| (XRPD analysis) | |
| Particle size | D₅₀: 61.1 μm |
| | D₉₀: 155.6 μm |
| Hygroscopicity (DVS) | Hygroscopic (≤16% weight increase upon |
| | equilibration at 80% RH at 25° C.) |

### Excipients

A pharmaceutical composition was formulated as a medicated enema solution formulation. The pharmaceutical composition comprised the following: a peptide having an amino acid sequence of: Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus (hereinafter referred to as “the peptide”), and the following excipients: Sodium phosphate monobasic monohydrate, sodium phosphate dibasic heptahydrate, sterile water for irrigation, sodium hydroxide, and phosphoric acid.

The excipients used to formulate the pharmaceutical composition comply with the United States Pharmacopeia (USP) and/or the National Formulary (NF) monographs. These excipients (listed in the FDA Inactive Ingredient Database) are selected to achieve adequate solubility and stability of the peptide drug substance.

### Example 3. Active Drug Substance: The Peptide

The pharmaceutical composition, in a unit dosage form having a concentration of 100 μg, 300 μg, 600 μg, 900 μg, 1800 μg, and 2500 μg, was formulated as an aqueous solution designed to be compounded at the clinical pharmacy and delivered rectally (e.g., as an enema). The enema vehicle is a 20 mM sodium phosphate buffer (pH=7.0): this enema vehicle was chosen due to the demonstrated solubility and chemical stability of the peptide drug substance in this media at the desired concentrations.

Each dose of pharmaceutical composition was formulated as a different concentration of the peptide drug substance, i.e., 100 μg, 300 μg, 600 μg, 900 μg, 1800 μg, or 2500 μg of a peptide of the present disclosure in the enema vehicle, to maintain a constant dose volume of 20 mL for all dosage strengths. To cover the unit doses required (100 μg, 300 μg, 600 μg, 900 μg, 1800 μg, and 2500 μg), the enema was prepared at concentrations 5.00 μg/mL, 15.0 μg/mL, 30.0 μg/mL, 45.0 μg/mL, 90.0 μg/mL, and 125 μg/mL of the peptide drug substance, in 20 mM sodium phosphate buffer (pH=7.0). Bulk enema solutions of the pharmaceutical compositions were compounded on the intended day of administration.

An in-use stability study was conducted to define the duration the pharmaceutical compositions can be used, without compromising the quality of the peptide, and by bracketing the solution concentrations to be used in the clinical study. The results of the in-use stability study are shown in the table below. Here, solutions were prepared at concentrations of 5.00 μg/mL and 125 μg/mL of the peptide drug substance in 20 mM sodium phosphate buffer (pH=7.0). Samples were stored in amber glass bottles with a PTFE-lined screw top. The bottles were stored at 25° C./Ambient with samples pulled at 0, 1, 3, and 7 days. Samples were tested for pH and a peptide assay. In all cases, pH and measured concentration were consistent and no change in the chromatographic profile were observed throughout the duration of the study.

TABLE 3
Stability study of the pharmaceutical composition.

| Concen- | Equivalent | | | | | |
|---|---|---|---|---|---|---|
| tration | Dose | | | | | |
| (μg/mL) | (μg) | Test | T = 0 | 1 day | 3 days | 7 days |
| 5.00 | 100 | pH | 6.94 | N/A | 6.89 | 6.97 |
| | | Peptide Assay | N/A | 96.7 | 99.7 | 100.2 |
| | | (% Initial) | | | | |
| 125 | 2500 | pH | 6.93 | N/A | 6.89 | 6.97 |
| | | Peptide Assay | N/A | 100.3 | 100.4 | 101.1 |
| | | (% Initial) | | | | |

### Example 4. Manufacturing Process Development

The pharmaceutical compositions comprising a peptide having an amino acid sequence of: Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus (hereinafter referred to as “the peptide”), and at strengths of 100 μg, 300 μg, 600 μg, 900 μg, 1800 μg, and 2500 μg of the peptide, were compounded at PPD clinical pharmacy (PPD Orlando Research Unit, 100 W Gore St. Suite 202, Orlando, FL 32806). A mock trial may be conducted prior to a clinical study. Here, assay and pH may be measured to demonstrate the effectiveness of the compounding procedure.

To create the aforementioned pharmaceutical compositions, the excipient, i.e., a 20 mM sodium phosphate buffer (pH=7.0), was first prepared by dissolving sodium phosphate monobasic monohydrate and sodium phosphate dibasic heptahydrate in water, mixing with a stir plate and magnetic stir bar. The pH was checked and adjusted via addition of sodium hydroxide or phosphoric acid, as needed.

The pharmaceutical composition was prepared at concentrations ranging from 5.00 μg/mL to 125 μg/mL of the peptide drug substance in 20 mM sodium phosphate buffer (pH=7.0). The aqueous solubility of the peptide drug substance at this pH is >1 mg/mL. Therefore, dissolution of the peptide drug substance in the enema vehicle was easily achieved utilizing a stir plate and magnetic stir bar. The specified dose volume of bulk pharmaceutical composition was be measured using a graduated syringe immediately before administering to the patient. A detailed description of the compounding process is provided below.

### Example 5. Container Closure System

Bulk pharmaceutical compositions may be stored in amber glass bottles with PTFE-lined screw tops. In-use stability data to support storage in these bottles is presented in Example 4. The proper dose volume may be measured and delivered using a 50 mL polypropylene syringe at the time of administration. The enema form of the pharmaceutical composition may be administered using the 50 mL syringe affixed with a 15-cm long PVC enema applicator with a thickness of 4.667 mm. A compatibility study was conducted in which 20 mL of each 100 μg and 2500 μg peptide (bracketed doses), was measured, and dispensed through the syringe and applicator. The assay of the pharmaceutical composition was demonstrated to be consistent before and after contact with the syringe and applicator.

### Example 6. Microbiological Attributes

The pharmaceutical composition will be administered on the day of compounding at the clinical pharmacy. The individual components of the peptide drug substance, salts, and water used in the compounded pharmaceutical composition solution are not expected to contribute to microbial growth when administered on the same day of preparation.

### Example 7. Batch Formula

The quantitative batch formula for compounding of the pharmaceutical composition enema vehicle is provided in Table 4 below, based on a 1 L batch size. The quantitative batch formula for compounding the pharmaceutical composition enema at unit dosage forms of 100 μg, 300 μg, 600 μg, 900 μg, 1800 μg, and 2500 μg was based on a 200 mL batch size is provided in Table 5. In some embodiments, batch size may vary depending on clinical need.

TABLE 4
Quantitative composition of enema vehicle. Here, the enema
vehicle is a 20 mM Sodium Phosphate Buffer, pH = 7.0).

| | Theoretical | | |
|---|---|---|---|
| | Quantity | | Quality |
| Component | per Batch (g) | Function | Standard |
| Sodium Phosphate Monobasic | 1.17 | Buffer Salt | USP |
| Monohydrate | | | |
| Sodium Phosphate Dibasic | 3.09 | Buffer Salt | USP |
| Heptahydrate | | | |
| Sterile Water for Irrigation | 1000^{a} | Solvent | USP |
| Sodium Hydroxide | N/A^{b} | pH Modifier | NF |
| Phosphoric Acid | N/A^{b} | pH Modifier | NF |

^{a}QS to 1. 0 L of water.
^{b}Sodium hydroxide and phosphoric acid are used to adjust the buffer pH as needed to 7.0 ± 0.2.

TABLE 5
Quantitative Composition of the pharmaceutical composition.
Quantitative Composition of the pharmaceutical composition at a concentration
of: 100 μg, 300 μg, 600 μg, 900 μg, 1800 μg, and 2500 μg /20 mL.

| Component | Composition | | | | | | | | | | | | |
|---|---|---|---|---|---|---|---|---|---|---|---|---|---|
| Enema Dose | 100 μg | | 300 μg | | 600 μg | | 900 μg | | 1800 μg | | 2500 μg | | Function |
| Sodium | 200 | mL | 200 | mL | 200 | mL | 200 | mL | 200 | mL | 200 | mL | Vehicle |
| Phosphate Buffer | | | | | | | | | | | | | |
| (pH 7.0) | | | | | | | | | | | | | |
| Peptide^{a} | 1.0 | mg | 3.0 | mg | 6.0 | mg | 9.0 | mg | 18.0 | mg | 25.0 | mg | Active |

^{a}= the actual quantity of peptide drug substance is corrected for purity.

### Example 8. Description of Manufacturing Process

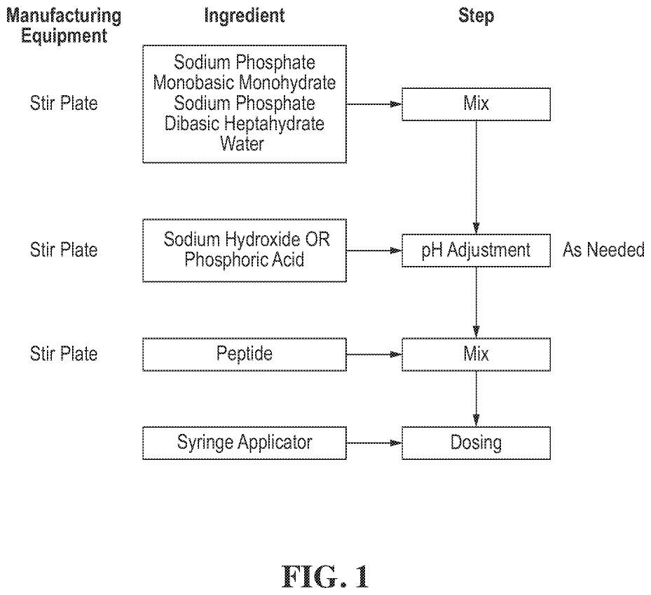

The pharmaceutical composition of the present disclosure was manufactured according to the process shown in FIG. 1. Briefly, a synthetic peptide was produced, having an amino acid sequence of: Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus (hereinafter referred to as “the peptide”), was produced, according to a stepwise solid-phase peptide chemistry, followed by a succession of steps involving the incorporation of the terminal amino acid, the cyclization step, the folding step, the terminal acetylation, extensive purification by preparative-scale chromatography, and a final isolation of the drug substance as a solid by precipitation.

Next, the compounding of the pharmaceutical composition was divided into two primary steps. First, sodium phosphate monobasic monohydrate and sodium phosphate dibasic heptahydrate were dissolved in water to generate a sodium phosphate buffer used as the enema vehicle. Sodium hydroxide or phosphoric acid may be added to ensure that the vehicle has a pH of 7.0±0.2. Next, the peptide drug substance was dissolved in the enema vehicle to generate the bulk enema. The bulk enema may be tested for pH and appearance, as described in the table below. The proper dose volume of bulk enema will be measured and delivered using a 50 mL polypropylene syringe at the time of administration.

There are no critical steps or key intermediates identified for the compounded formulation process. The pharmaceutical composition is evaluated for appearance and pH as stated in the compounding batch records. Appearance testing is performed visually. The pH is analyzed based on USP <791>.

TABLE 6
Pharmaceutical composition and placebo to match.

| Test | Method | Acceptance Criteria |
|---|---|---|
| Appearance | Visual | Clear, colorless solution free of |
| | | particulates |
| pH | USP <791> | 7.0 ± 0.2 |

### Example 9. Control of Drug Product

A validation of analytical procedures was not applicable. For batch analysis, the pharmaceutical composition was compounded at the clinical pharmacy per the procedure described above. The formulation composition of each concentration of the peptide in enema form is likewise described above. The compounded formulation will be used for a Phase 1 clinical trial and tested according to Example 8.

There are no related impurities specific to the drug product. All impurities arise from the drug substance. Impurities are discussed in the example below.

### Example 10. Characterization of Impurities

Two categories of impurities were considered: peptide and non-peptide related impurities. The impurities derived from the starting materials, side-products, residual starting materials, isomers (including stereoisomers), multimers and degradation products, are peptide-related impurities.

All other impurities, from residual solvents, elemental impurities, reagents and their adducts, are non-peptide related impurities. ICH Q3A (R2) is not applicable as synthetic peptides were excluded. Separately, the European Pharmacopoeia (current edition) monograph 2034 defines thresholds for impurities identification and qualification at 0.5% and 1.0%, respectively. ICH guidelines were applicable to elemental impurities and residual organic volatiles.

### Related Substances

Impurities Derived from the Starting Materials

Protected amino acid starting materials were designated as starting materials and were produced by a synthetic pathway from elementary precursors. Key attributes were the identity, optical purity, and related substances. Related substances included unprotected variants, di-amino acid variants or, in the case of Fmoc-protected amino acids, where the protecting group was introduced as a hydroxysuccinimic ester, the beta-alanine insertion variants. These impurities, if present, would be liable to react in a similar way as the authentic starting material and lead to a related substance.

Consequently, all starting materials were subject to incoming testing which provided for robust control towards the corresponding impurities. The impurity profile for the drug substance obtained from the initial GMP production runs was investigated accordingly. At the outcome of the non-GMP process development and at the scale investigated, none of these potential impurities were found to be of concern.

### Process-Related Peptide Impurities

The peptide drug substance was manufactured according to a stepwise solid-phase peptide chemistry followed by a succession of steps involving the incorporation of the terminal amino acid, the cyclization step, the folding step, the terminal acetylation, extensive purification by preparative-scale chromatography, and a final isolation of the drug substance as a solid by precipitation.

Each chemical step was liable to terminate before completion, leading to potential deletion peptides (sequence missing one or more amino acids) or double insertion peptides (sequence including a double incorporation of one or more amino acids). Non-cyclized linear peptide variants or multimer peptides could also occur, as well as non-acetylated or over-acetylated variants.

Specific in-process controls were implemented to monitor all corresponding reaction steps, control the quality of the intermediates (coupling and deprotection completeness during solid-phase synthesis, all subsequent chemical steps including C-terminal residue incorporation, cyclization, folding and acetylation), and select the fractions from the two independent chromatographic purification steps. Partial racemization could potentially also occur during the individual amino-acid incorporation steps.

The selection of the chemistry associated with the coupling steps (N-terminal urethane protecting groups and coupling in the presence of Oxyma®, a protecting agent against racemization, as well as extensive chromatographic purification under two orthogonal conditions, and extensive purging of all potential process-related impurities was observed at the outcome of the non-GMP process development and at the scale investigated. Here also, the impurity profile for the drug substance obtained from the initial GMP production runs was investigated accordingly.

### Degradation Products

No degradation pathway was identified that would lead to any concern related to the stability of process intermediates or even the final isolated peptide drug substance during the conditions of manufacturing.

This observation complements the continued evaluation of the product quality demonstrated in the stability studies below.

### Non-Peptide Process Impurities and Process Impurities

### Reagents

In a solid-phase route of synthesis, excess reagents were washed off the resin-bound growing peptide chain at each step. Hence, non-peptidic impurities from the upstream steps were unlikely to be present. In the peptide drug substance downstream process, extensive work-up extraction and washing operations were conducted, and two successive chromatographic purification steps were applied.

Consequently, the focus was on the ionizable elements used in the final process steps, in particular trifluoroacetic acid, acetic acid and ammonia, which could potentially form a salt form with the drug substance. They are systematically assessed during the final QC batch release testing.

### Solvents

All solvents used in the upstream process steps (solid-phase synthesis and subsequent cleavage, deprotection, coupling, cyclization and folding) were completely soluble or miscible in the eluents used during the chromatographic purification steps. They are highly likely to be purged.

The solvent used in the final process steps were specifically analyzed and the levels found are reported during the final QC batch release testing.

### Elemental Impurities

Considering the extensive use of stainless-steel equipment throughout the successive manufacturing steps, as well as the implementation of one palladium-catalyzed deprotection step, a systematic evaluation of all class 1 and 2A heavy metals (according to the classification of ICH Q3D) in addition to palladium was conducted as part of the final QC batch release testing.

### Example. 11 Justification of Specifications

The pharmaceutical composition comprising a peptide having an amino acid sequence of: Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus (hereinafter referred to as “the peptide”), or a placebo, were evaluated for appearance and pH. The justification of these tests for the pharmaceutical composition is described below and is appropriate for the lifecycle of the material based on USP <1168>, Compounding for Phase I Investigational Studies.

Appearance by visual inspection was checked after compounding pharmaceutical composition bulk enema. The acceptance criteria of “Clear, colorless solution free of particulates” for the pharmaceutical composition, was consistent with compounded formulations. The pH was analyzed after compounding pharmaceutical composition bulk enema. The pH test was performed per USP <791>. The acceptance criteria of 7.0±0.2 are set to ensure the stability of the drug product.

### Example 12. Stability Summary and Conclusion

Supportive in-use stability data was obtained for the pharmaceutical composition at the bracketed concentrations of 5.00 μg/mL and 125 μg/mL (drug substance lot 2011165) in the enema vehicle, equivalent to doses of 100 μg and 2500 μg, respectively. These solutions were stored at 25° C./Ambient up to seven days (0, 1, 3, and 7 days), packaged in amber glass bottles with PTFE-lined screw caps. The storage at 25° C./Ambient for up to seven days represents the worst-case scenario for the stability of the peptide compounded solution as the administration is intended to be on the day of compounding.

The results from the in-use study are consistent with the proposed drug product specifications described above. An expiry date of 1 day at 25° C./Ambient for the compounded solution was established based on the accumulated supportive in-use stability data provided herein.

### Example 13. Development of Rectal Foam Formulation

A pharmaceutical rectal foam composition was developed and evaluated. First, a foam emulsion product was developed, operable to deliver a minimum of 30 μg, and a maximum of 3,000 μg, of a peptide having the following amino acid sequence: Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus (hereinafter referred to as “the peptide”), in a volume suitable for adult administration.

In some embodiments, a pharmaceutical composition comprising the peptide of the present disclosure, formulated as a foam emulsion product, can be used for the treatment of visceral pain, e.g., lower colon and rectal pain. In some embodiments, a pharmaceutical composition comprising the peptide of the present disclosure, formulated as a foam emulsion product and can be packaged in a 30-g aluminum aerosol can with a metered dose valve.

The following considerations were taken into account when developing the pharmaceutical rectal foam composition. The peptide at pH=7.0 was determined to be stable and had a drug solubility of greater than 26.4 mg/mL, which was well above the proposed development concentration range. From this, development work settled on the pH range of 6.5 to 7.5. To achieve this pH range for the bulk product; accordingly, a phosphate buffer system was chosen.

For the preservative system of the pharmaceutical composition, the preservatives Methylparaben and Propylparaben were chosen. Based on the FDA's Inactive Ingredient Guide (IIG), use of Propylparaben in a rectal solution is about 0.02% and Methylparaben is about 0.18% in an enema product. A final consideration was the temperature sensitivity of the peptide. The manufacturing process would have to consider adding the peptide at a temperature below 30° C. Due to the limited supply of the peptide, the initial prototypes formulated were Placebo bulk products.

### Formulation Risk Assessment

### Peptide Stability:

Because the active pharmaceutical ingredient (API) of the pharmaceutical rectal foam composition is a peptide, its exposure to extreme temperatures was limited. Thus, the side phase and the final mix phase were maintained at 30° C. or below when the peptide was added.

Some peptides can be sensitive to high shear mixing. Accordingly, high shear was avoided after the peptide was introduced to the formula. Based on the foregoing examples, the ideal pH range for the peptide is about 7.0. Therefore, the side phase and the final product were monitored and adjusted to pH of 6.5 to 7.5. Excipient compatibility with the peptide was a potential risk that has not been addressed to this point; however, the risk is minimized as noted below. Additionally, the informal stability data indicates that there is no significant incompatibility between the bulk formula and packaging components and the peptide.

### Foam Performance:

Foam stability can present a risk because the peptide may destabilize the foam or it may be difficult to identify good propellant-formulation combinations. And, delivered dose uniformity may be impacted by the propellant-formulation combinations.

However, notwithstanding foregoing risks, the examples below show that the pharmaceutical rectal foam composition comprising a peptide having the following amino acid sequence: Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus, was stable, and the foam was able to perform.

### Example 14. Pharmaceutical Rectal Foam Prototypes

Pharmaceutical rectal foam composition prototypes were evaluated. In the examples below, for each prototype, the peptide used was a peptide having the following amino acid sequence: Cys₁-Cth₂-Glu₃-Leu₄-Cys₅-Cys₆-Asn₇-Val₈-Ala₉-Cys₁₀-Tyr₁₁-Gly 12-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus (hereinafter referred to as “the peptide”).

### Prototype A

Table 7 below provides the formula for the initial prototype (Lot 1769-07 27R01), which became Prototype A. This table provides the formula concentration for each ingredient.

TABLE 7
Formulation of pharmaceutical rectal foam Prototype A. This
table provides the formula concentration for each ingredient.
The column named IIG shows the FDA's Inactive Ingredient
Guide (IIG) limit for a rectal or rectal foam product and
the function of each ingredient used in the formula.

| Prototype A Ingredients | % w/w | IIG Limit | Function |
|---|---|---|---|
| Purified Water USP/EP | 77.5495 | N/A | Solvent/Diluent |
| Propylene Glycol USP | 10.0000 | 47.04% | Solvent/Diluent |
| Sodium Phosphate Dibasic | 0.1640 | 0.50% | pH modifier |
| USP | | | |
| Sodium Dihydrogen | 0.1165 | 0.50% | pH modifier |
| Phosphate Monohydrate | | | |
| USP | | | |
| Disodium EDTA USP | 0.0500 | 0.09% | Chelating agent |
| Methylparaben NF | 0.1000 | 0.10% | Preservative |
| Light Mineral Oil NF | 6.0000 | N/A | Solvent/Diluent |
| Isopropyl Myristate NF | 0.5000 | 7.90% | Solvent/Diluent |
| White Petrolatum USP | 1.0000 | 75% | Emollient |
| Polyoxyl 20 Cetostearyl | 2.5000 | 5.20% | Stiffening Agent |
| Ether NF | | | |
| Cetyl Alcohol NF | 1.0000 | 1.16% | Stiffening Agent |
| Stearyl Alcohol NF | 1.0000 | 1.50% | Emulsifier |
| Propylparaben NF | 0.0200 | 0.02% | Preservative |
| Total | 100.00 | | |

“NF” = National Formulary.
“USP” = United States Pharmacopeia.

The following is the lab batch process for pharmaceutical rectal foam Prototype A:

### Aqueous Phase—Prototype A

The Aqueous Phase was prepared in a glass beaker into which Purified Water and Propylene Glycol were added; this was placed under an overhead lab mixer with a 3-inch 3-prong mixing blade and mixed at 425-432 RPM at room temperature conditions, while Sodium Phosphate, Sodium Dihydrogen Phosphate and Disodium EDTA were added. Mixing continued until all solids were visually observed to be dissolved (approximately 12 minutes). A sample was removed to perform an in-process pH check, wherein the pH was 7.05. While this was a Placebo, approximately ⅓ of the Aqueous Phase was removed to prepare the “Active” Phase. The remaining ⅔ was mixed at 274 RPM and heated to 75-80° C., which took about 30 minutes.

Methylparaben was added and mixed until all solids were visually observed to be dissolved, while maintaining 75-80° C., which took about 5 minutes. The phase was a uniform, clear solution with no solid particles observed.

### Oil Phase—Prototype A

The Oil Phase was prepared in a glass beaker into which the Oil Phase ingredients: Light Mineral Oil, Isopropyl Myristate, White Petrolatum, Polyoxyl 20 Cetostearyl Ether, Cetyl Alcohol, Stearyl Alcohol and Propylparaben were added. The Oil Phase was placed under an overhead lab mixer with a 1.5-inch 3-prong mixing blade and was mixed at 400 RPM while heating to 75-80° C. until all waxy solids were observed as melted, a process that took about 9 minutes. The Oil phase was a uniform solution with no solid particles or un-melted waxy particles observed.

### Emulsification Phase—Prototype A

Next, the Oil Phase was added to the Aqueous Phase. Mixing continued at 552 RPM with a 3-inch 3-prong mixing blade until visually uniform while maintaining 75-80° C. for about 15 minutes. This mixture was then cooled at 335 RPM to 30° C. or below. The final temperature was 28° C. This phase was observed to be a uniform, thin white to off-white milky cream emulsion.

### “Active” Phase—Prototype A

The “Active” Phase can be prepared with ⅓ Aqueous Phase at room temperature wherein the peptide can be added. Mixing can continue until all solids are visually observed to be dissolved. Because Prototype A is a Placebo formula, the ⅓ Aqueous Phase can be added to the base emulsion. When preparing an active formulation comprising the peptide of the present disclosure, this ⅓ aqueous phase with peptide can be added to the base emulsion. Mixing continued with a 3-inch 3-prong mixing blade at 467-671 RPM for 17 minutes, and until visually uniform. The final temperature was 25° C. and a pH check was 7.00. The final batch was observed to be a uniform, thin white to off-white milky cream emulsion.

### Prototype B

Table 8 below provides the formula for the prototype Lot 1769-07 28R01, which became Prototype B. This table provides the formula concentration for each ingredient.

TABLE 8
Formulation of pharmaceutical rectal foam Prototype B. This table provides
the formula concentration for each ingredient. The column named IIG shows
the FDA's Inactive Ingredient Guide (IIG) limit for a rectal or rectal
foam product and the function of each ingredient used in the formula.

| Prototype B Ingredients | % w/w | IIG limit | Function |
|---|---|---|---|
| Purified Water USP/EP | 79.5695 | N/A | Solvent/Diluent |
| Propylene Glycol USP | 16.4800 | 47.04% | Solvent/Diluent |
| Sodium Phosphate Dibasic USP | 0.1640 | 0.50% | pH modifier |
| Sodium Dihydrogen Phosphate Monohydrate USP | 0.1165 | 0.50% | pH modifier |
| Disodium EDTA USP | 0.0500 | 0.09% | Chelating agent |
| Methylparaben NF | 0.1000 | 0.10% | Preservative |
| Emulsifying Wax NF | 1.4000 | 1.50% | Emulsifier |
| Polyoxylene (10) Stearyl Ether NF | 1.4000 | 5.20% | Stiffening Agent |
| Cetyl Alcohol NF | 0.7000 | 1.16% | Stiffening Agent |
| Propylparaben NF | 0.0200 | 0.02% | Preservative |
| Total | 100.00 | | |

“NF” = National Formulary. “USP” = United States Pharmacopeia.

The following is the lab batch process for pharmaceutical rectal foam Prototype B

### Aqueous Phase—Prototype B

The Aqueous Phase was prepared in a glass beaker into which Purified Water was added. This was placed under an overhead lab mixer with a 3-inch 3-prong mixing blade, which was mixed at 462 RPM and at room temperature conditions while Sodium Phosphate, Sodium Dihydrogen Phosphate and Disodium EDTA were added. Mixing continued at 462 RPM until all solids were visually observed to be dissolved, which took 18 minutes. A sample was removed to perform an in-process pH check, where the pH was 6.96. While this was a Placebo, approximately ⅓ of the Aqueous Phase was removed to prepare the “Active” Phase. The remaining ⅔ was mixed at 302 RPM and heated to 68-73° C. which took 20 minutes.

Methylparaben was added and mixed at 304 RPM for 11 minutes and until all solids were visually observed to be dissolved, while maintaining 68-73° C. The phase was a uniform, clear solution with no solid particles observed.

### Oil Phase—Prototype B

The Oil Phase was prepared in a glass beaker into which the following Oil Phase ingredients were added: Propylene Glycol, Cetyl Alcohol, Emulsifying Wax, Polyoxylene 10 Stearyl Ether and Propylparaben. This mixture was placed under an overhead lab mixer with a 1.5-inch 3-prong mixing blade and was mixed at 400 RPM while heating to about 68-73° C., and until all waxy solids were observed as melted which took about 8 minutes. The phase was a uniform solution with no solid particles or un-melted waxy particles observed.

### Emulsification Phase—Prototype B

Next, the Oil Phase was added to the Aqueous Phase. Mixing continued at 550 RPM with a 3-inch 3-prong mixing blade for 11 minutes, and until visually uniform while maintaining 6873° C. This mixture was then cooled at 286-312 RPM to 30° C. or below. The final temperature was 27° C. This phase was observed to be a uniform, thin white to off-white milky cream emulsion.

### “Active” Phase—Prototype B

The “Active” Phase can be prepared with ⅓ Aqueous Phase at room temperature where the peptide can been added. Mixing can continue until all solids are visually observed to be dissolved. Because this is a Placebo formula, the ⅓ Aqueous Phase can be added to the base emulsion. For preparing active formulation, this ⅓ aqueous phase comprising the peptide of the present disclosure can be added to the base emulsion. Mixing continued at 470 RPM with a 3-inch 3-prong mixing blade for 12 minutes, and until visually uniform. The final temperature was 25° C., and a final pH check was 7.06. The final batch was observed to be a uniform, thin white to off-white milky cream emulsion.

### Prototype C

Table 9 below provides the formula for the prototype Lot 1769-0915R01, which became Prototype C. This table provides the formula concentration for each ingredient.

TABLE 9
Formulation of pharmaceutical rectal foam Prototype C. This table provides
the formula concentration for each ingredient. The column named IIG shows
the FDA's Inactive Ingredient Guide (IIG) limit for a rectal or rectal
foam product and the function of each ingredient used in the formula.

| Prototype C Ingredients | % w/w | IIG Limit | Function |
|---|---|---|---|
| Purified Water USP/EP | 77.5495 | N/A | Solvent/Diluent |
| Propylene Glycol USP | 10.0000 | 47.04% | Solvent/Diluent |
| Sodium Phosphate Dibasic USP | 0.1640 | 0.50% | pH modifier |
| Sodium Dihydrogen Phosphate Monohydrate USP | 0.1165 | 0.50% | pH modifier |
| Disodium EDTA USP | 0.0500 | 0.09% | Chelating agent |
| Methylparaben NF | 0.1000 | 0.10% | Preservative |
| Light Mineral Oil NF | 7.0000 | N/A | Solvent/Diluent |
| White Wax NF | 0.5000 | 2.00% | Emollient |
| Mono - & Di-Glycerides | 2.5000 | 3.27% | Stiffening Agent |
| Cetyl Alcohol NF | 1.0000 | 1.16% | Stiffening Agent |
| Stearyl Alcohol NF | 1.0000 | 1.50% | Emulsifier |
| Propylparaben NF | 0.0200 | 0.02% | Preservative |
| Total | 100.00 | | |

“NF” = National Formulary. “USP” = United States Pharmacopeia.

The following is the lab batch process for pharmaceutical rectal foam Prototype C

### Aqueous Phase—Prototype C

The Aqueous Phase was prepared in a glass beaker into which Purified Water and

Propylene Glycol were added. This was placed under an overhead lab mixer with a 3-inch 3-prong mixing blade. This was mixed at 305 RPM and at room temperature condition while Sodium Phosphate, Sodium Dihydrogen Phosphate and Disodium EDTA were added. Mixing continued at 305 RPM for 8 minutes and until all solids were visually observed to be dissolved. A sample was removed to perform an in-process pH check, where the pH was 7.05. While this was a Placebo, approximately ⅓ of the Aqueous Phase was removed to prepare the “Active” Phase. The remaining ⅔ was mixed at 204-253 RPM and heated to 75-80° C., which took 24 minutes.

Methylparaben was added and mixed at 253 RPM for 5 minutes and until all solids were visually observed to be dissolved, while maintaining 75-80° C. The phase was a uniform, clear solution with no solid particles observed.

### Oil Phase—Prototype C

The Oil Phase was prepared in a glass beaker into which the following Oil Phase ingredients were added: Light Mineral Oil, White Wax, Mono-& Di-Glycerides, Cetyl Alcohol, Stearyl Alcohol and Propylparaben. This mixture was placed under an overhead lab mixer with a 1.5-inch 3-prong mixing blade, and was mixed at 560 RPM for 3 minutes while heating to 75-80° C., and until all waxy solids were observed as melted. The resulting phase was a uniform solution with no solid particles or un-melted waxy particles observed.

### Emulsification Phase—Prototype C

Next, the Oil Phase was added to the Aqueous Phase. Mixing continued at 393-396 RPM with a 3-inch 3-prong mixing blade for 6 minutes and until visually uniform while maintaining 75-80° C. This was mixed at 265-515 RPM and cooled to 30° C. or below. The final temperature was 28° C. The phase was observed to be a uniform, thin white to off-white milky cream emulsion.

### “Active” Phase-Prototype C

The “Active” Phase would be prepared with ⅓ Aqueous Phase at room temperature during which point the peptide of the present disclosure can be added. Mixing can continue until all solids are visually observed to be dissolved. Because this was a Placebo formula, the ⅓ Aqueous Phase was added to the base emulsion. For preparing active formulation this ⅓ aqueous phase comprising the peptide can be added to the base emulsion. Mixing continued with a 3-inch 3-prong mixing blade at 459-483 RPM for 15 minutes and until visually uniform. The final temperature was 26° C. and a final pH check was 7.00. The final batch was observed to be a uniform, thin white to off-white milky cream emulsion.

### Example 15. Propellants

The table below provides the propellants used for consideration in the pharmaceutical rectal foam composition. This table provides the propellant name, the PSI specification range for the propellant and the composition and ratios in these propellants.

TABLE 10
Propellants for the pharmaceutical rectal foam composition.

| Propellant | PSI | Composition(s)/ratios (%) |
|---|---|---|
| DME | 49-55 | Dimethyl Ether/n-Butane (53/47) |
| A17 | 15-19 | n-Butane (>97) |
| A31 | 29-33 | Isobutane (>95) |
| AP35 | 33-37 | Isobutane (83-89)/n-Butane(5-9)/Propane(5-9) |
| A46 | 44-48 | Isobutane (76.6-88)/Propane (12-21.9) |
| A48 | 46-50 | n-Butane (30-60)/Isobutane (15-30)/Propane (20-45) |
| A70 | 68-72 | Isobutane (46-64)/Propane (35.7-52.3)/n-Butane (0-3) |
| AP70 | 68-72 | Propane (49.8-59.8)/n-Butane (22.3-32.3)/Isobutane(12.9-22.9) |

Bulk product was packaged in 30-g aluminum aerosol cans with a metered dose valve. Each can contained 31 g of bulk product, and were gassed with approximately 2.8 g (2.3 g-3.2 g) a given propellant. For Prototype A, each propellant actuated well and provided a white foam with a solid and creamy consistency. For Prototype B, only AP35 and AP70 actuated well and provided a white foam with a solid and creamy consistency. For Prototype C, none of the propellants produced acceptable foam actuations. Based on this performance, Prototype C was removed from consideration.

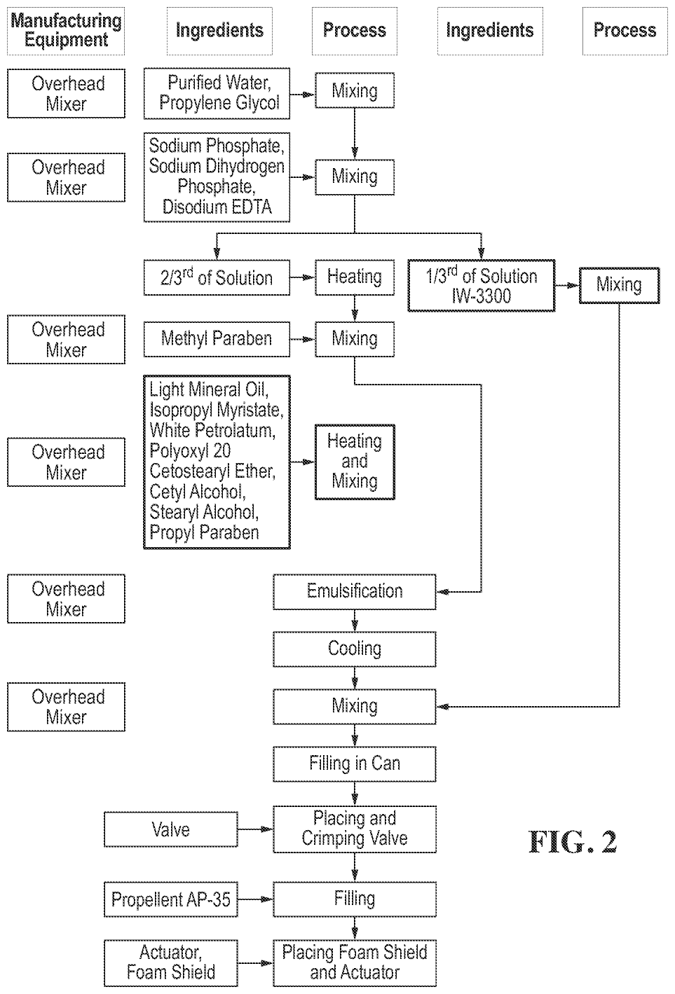

For both Prototypes A and B, the propellants AP35 and AP70 actuated well and provided a white foam with a solid and creamy consistency. Due to the limited supply of peptide only one propellant, AP70, was chosen for further development studies. The table below provides additional lab batches for evaluation. For these batches, the peptide Lot B01511P0 23 was used for the Active batches below. The Bulk pH was performed at the completion of compounding of the lab batch. The appearance or description was based on the visual observation of the actuated foam. The foam height evaluation in the table below was the average of three separate actuations using the apparatus as shown in FIG. 2. The pH of the foam product was performed on a single actuation dissolved in 100 mL of a 5% Methanol in Water solution. Samples of bulk and canned products were subsequently analyzed for method development.

TABLE 11
Pharmaceutical rectal foam composition evaluations. Prototypes
A and B were evaluated. The table below provides additional lab
batches for evaluation. The column named “Strength” indicates
the corresponding dose. “Bulk pH” indicates the pH as
determined at the completion of compounding of the lab batch.
“Appearance” indicates whether there was an appearance
of a white foam as determined via visual observation of the actuated
foam. Foam height evaluation was the average of three separate
actuations. The pH of the foam product was performed on a single
actuation dissolved in 100 mL of a 5% Methanol in Water solution.

| | | | | | | |
|---|---|---|---|---|---|---|
| | | Batch | Bulk | Appear- | Foam | |
| Lot | Strength | size (g) | pH | ance | height (mL) | pH |

Pharmaceutical rectal foam composition - Prototype A

| 1769-0831R01 | Placebo | | 1000 | 7.0 | Pass | 30 | 6.9 |
|---|---|---|---|---|---|---|---|
| 1769-0901R01 | 30 | μg | 1000 | 7.0 | Pass | 20 | 6.9 |
| 1769-0902R01 | 3000 | μg | 700 | 6.8 | Pass | 28 | 6.9 |

Pharmaceutical rectal foam composition - Prototype B

| 1769-0831R02 | Placebo | | 1000 | 7.1 | Pass | 15 | 6.8 |
|---|---|---|---|---|---|---|---|
| 1769-0901R02 | 30 | μg | 1000 | 7.0 | Pass | 11 | 6.8 |
| 1769-0902R02 | 3000 | μg | 700 | 6.9 | Pass | 13 | 6.8 |

At this point good foam height results were deemed sufficient for the next steps of product development. This indicated that the product was performing as desired when using the metered dose actuator. Delivered dose measurements were not performed.

Additional lab batches for the pharmaceutical rectal foam compositions were evaluated. The Placebo batches contained no Active peptide. The no Parabens batches contained peptide at the 30 μg concentration, but no Preservative. The vehicle batches contained neither Active nor preservatives. For the Active batches the 30 μg concentration was the only concentration submitted.

TABLE 12
Additional Pharmaceutical rectal foam composition evaluations.

| | | | |
|---|---|---|---|
| Lot | Strength | Batch size (g) | Final pH |

Pharmaceutical rectal foam composition - Prototype A

| | | | |
|---|---|---|---|
| 1797-1005R01 | 30 μg | 1000 | 7.1 |
| 1797-1008R03 | No Parabens | 500 | 7.1 |
| 1797-1008R04 | Placebo | 500 | 7.0 |
| 1797-1009R01 | Vehicle | 1000 | 7.1 |

Pharmaceutical rectal foam composition - Prototype B

| | | | |
|---|---|---|---|
| 1797-1006R01 | 30 μg | 1000 | 7.1 |
| 1797-1008R01 | Vehicle | 1000 | 7.2 |
| 1797-1008R02 | No Parabens | 500 | 7.1 |
| 1797-1009R02 | Placebo | 500 | 7.2 |

### CONCLUSION

In conclusion, two foam emulsion prototypes were developed, operable to deliver a minimum of 30 μg and a maximum of 3,000 μg of the active pharmaceutical ingredient (API), i.e., the peptide of the present disclosure, in a 30-g aluminum aerosol can with a metered dose valve.

### Example 16. Development Process for Stability Assessment

The present example evaluated the stability of the pharmaceutical rectal foam compositions: Prototypes A and B, developed as described in the examples above.

Briefly, the two foam Prototypes were created to deliver a minimum of 30 μg and a maximum of 3,000 μg of the peptide having the following amino acid sequence: Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; and Cys₅ and Cys₁₃, are connected by disulfide bonds; wherein Cth₂ and Cys₁₀ are connected by a thioether bond; and wherein the peptide has an acetylated N-terminus, and an unmodified C-terminus (hereinafter referred to as “the peptide”), in a volume suitable for adult administration. This example covers the informal stability for Prototypes A and B produced in the Examples above, and the formulation enhancement work to Prototype A, which became a Prototype for further consideration.

### Prototype A

The following describes the laboratory batch process for generating Prototype A lab batches.

### Aqueous Phase—Prototype A

The Aqueous Phase was prepared in a glass beaker into which Purified Water and Propylene Glycol were added. This was placed under an overhead lab mixer with a 3-inch 3-prong mixing blade and mixed at room temperature conditions. Sodium Phosphate, Sodium Dihydrogen Phosphate and Disodium EDTA were added. Mixing continued at 346-400 RPM until all solids were visually observed to be dissolved, which took about 8-15 minutes. A sample was removed to perform an in-process pH check, where the pH was to be between 6.5-7.5. Approximately ⅓ of the Aqueous Phase was removed to prepare the Active Phase. The remaining ⅔ of the Aqueous Phase was heated to 75-80° C.

Methylparaben was added and mixed at 346-400 RPM until all solids were visually observed to be dissolved, a process taking about 10 minutes, while maintaining 75-80° C. The phase was observed to be a uniform, clear solution with no solid particles present.

### Oil Phase—Prototype A

The Oil Phase was prepared in a glass beaker into which the following Oil Phase ingredients were added: Light Mineral Oil, Isopropyl Myristate, White Petrolatum, Polyoxyl 20 Cetostearyl Ether, Cetyl Alcohol, Stearyl Alcohol and Propylparaben. This mixture was then placed under an overhead lab mixer with a 1.5-inch 3-prong mixing blade, and was mixed at 600-800 RPM while heating to 75-80° C., and until all waxy solids were observed as melted. The phase was observed to be a uniform solution with no solid particles or un-melted waxy particles present.

### Emulsification Phase—Prototype A

Next, the Oil Phase was added to the Aqueous Phase. Mixing continued at 400-600 RPM with a 3-inch 3-prong mixing blade until visually uniform for about 10-20 minutes while maintaining 75-80° C. This mixture was then cooled to 30° C. or below. The phase was observed to be a uniform, thin white to off-white milky cream emulsion.

### Active Phase—Prototype A

The Active Phase was prepared with ⅓ Aqueous Phase described above, at room temperature. The peptide of the present disclosure was adjusted for potency and added. For the Placebo batch, no peptide was added. Mixing continued with a ½ inch 3 prong mixing blade at 600-1040 RPM until all solids were visually observed to be dissolved, which took about 4-22 minutes. The Active Phase was then added to the base emulsion. Mixing continued with a 3-inch 3 prong mixing blade at 500 RPM until visually uniform, mixing took about 10-15 minutes. A final pH check was performed on the Active Phase, and determined to be in the range 6.5-7.5. The final batch was observed to be a uniform, thin white to off-white milky cream emulsion.

### Packaging—Prototype A

For each batch of Prototype A, 31 g of bulk product was filled into 35 mm×64 mm cans, with a PPG liner. Each canister was crimped with a 1-inch valve. AP-70 propellant was added at 2.8-3.2 g per can. The cans were then topped with the foam shied, and 1.35 mL metering head. Cans of Prototype A were placed at 5° C. and 25° C. for informal stability analysis. The table below provides the lab batch Lots for Prototype A.

TABLE 13
Prototype A Bulk formulation. Amounts are shown in grams.

| | Lot 1797-1109R02 | | Lot 1797-1111R01 | | Lot 1797-1111R02 | |
|---|---|---|---|---|---|---|
| | Placebo | | 30 μg/dose | | 3000 μg/dose | |
| Ingredient | % w/w | Amounts | % w/w | Amounts | % w/w | Amounts |
| Purified Water USP/EP* | 77.5495 | 1085.69 | 77.5469 | 775.47 | 77.2981 | 541.03 |
| Propylene Glycol USP | 10.0000 | 140.01 | 10.0000 | 100.01 | 10.0000 | 70.00 |
| Sodium Phosphate Dibasic | 0.1640 | 2.30 | 0.1640 | 1.64 | 0.1640 | 1.15 |
| USP | | | | | | |
| Sodium Dihydrogen | 0.1165 | 1.63 | 0.1165 | 1.17 | 0.1165 | 0.8159 |
| Phosphate Monohydrate | | | | | | |
| USP | | | | | | |
| Disodium EDTA USP | 0.0500 | 0.7022 | 0.0500 | 0.5001 | 0.0500 | 0.3509 |
| Methylparaben NF | 0.1000 | 1.40 | 0.1000 | 1.00 | 0.1000 | 0.7007 |
| Light Mineral Oil NF | 6.0000 | 84.00 | 6.0000 | 60.03 | 6.0000 | 42.01 |
| Isopropyl Myristate NF | 0.5000 | 7.01 | 0.5000 | 5.01 | 0.5000 | 3.50 |
| White Petrolatum USP | 1.0000 | 14.17 | 1.0000 | 10.00 | 1.0000 | 7.03 |
| Polyoxyl 20 Cetostearyl | 2.5000 | 35.00 | 2.5000 | 25.00 | 2.5000 | 17.50 |
| Ether NF | | | | | | |
| Cetyl Alcohol NF | 1.0000 | 14.00 | 1.0000 | 10.01 | 1.0000 | 7.00 |
| Stearyl Alcohol NF | 1.0000 | 14.00 | 1.0000 | 10.00 | 1.0000 | 7.00 |
| Propylparaben NF | 0.0200 | 0.2804 | 0.0200 | 0.2023 | 0.0200 | 0.1409 |
| Peptide (adjusted for | NA | NA | 0.00260 | 0.0269 | 0.25140 | 1.7600 |
| potency) | | | | | | |
| Total | 100.00 | 1400 | 100.00 | 1000 | 100.00 | 700 |

NA = not applicable.
*water can be adjusted to keep dose weight constant over different strengths.

### Prototype B

The following is the laboratory batch process for Prototype B lab batches.

### Aqueous Phase—Prototype B

The Aqueous Phase was prepared in a glass beaker into which Purified Water was added. This was placed under an overhead lab mixer with a 3-inch 3-prong mixing blade. This phase was mixed at room temperature conditions, while Sodium Phosphate, Sodium Dihydrogen Phosphate and Disodium EDTA were added. Mixing continued at 292-380 RPM until all solids were visually observed to be dissolved (about 3-17 minutes). A sample was removed to perform an in-process pH check, where the pH was to be between 6.5-7.5. Approximately ⅓ of the Aqueous Phase was removed to prepare the Active Phase. The remaining ⅔ was heated to 68-73° C. Methylparaben was added and mixed at 292-380 RPM for 8-10 minutes and until all solids were visually observed to be dissolved, while maintaining 68-73° C. The resulting phase was a uniform, clear solution with no solid particles observed.

### Oil Phase—Prototype B

The Oil Phase was prepared in a glass beaker into which the following Oil Phase ingredients were added: Propylene Glycol, Cetyl Alcohol, Emulsifying Wax, Polyoxylene 10 Stearyl Ether and Propylparaben. This phase was placed under an overhead lab mixer with a 1.5-inch 3-prong mixing blade, and was mixed 600-960 RPM while heating to 68-73° C., and until all waxy solids were observed as melted. The resulting phase was a uniform solution with no solid particles or un-melted waxy particles observed.

### Emulsification Phase—Prototype B

Next, the Oil Phase was added to the Aqueous Phase. Mixing continued at 400-607 RPM with a 3-inch 3-prong mixing blade for 10-17 minutes and until visually uniform, while maintaining 68-73° C. This mixture was then cooled to 30° C. or below. The final temperature was 30° C. or below. The resulting phase was observed to be a uniform, thin white to off-white milky cream emulsion.

### Active Phase—Prototype B

The Active Phase was prepared with ⅓ Aqueous Phase from above at room temperature. Next, the peptide of the present disclosure was added. Mixing continued at 600-880 RPM for 5-12 minutes, and until all solids were visually observed to be dissolved. The Active Phase was added to the base emulsion. Mixing continued with a 3-inch 3 prong mixing blade at 400-600 RPM for 1015 minutes and until visually uniform. The final pH check was in the range 6.5-7.5. The final batch was observed to be a uniform, thin white to off-white milky cream emulsion.

### Packaging—Prototype B

For each batch of Prototype B, 31 g of bulk product was filled into 35 mm×64 mm cans with a PPG liner. Each canister was crimped with a 1-inch valve. AP-70 propellant was added at 2.8-3.2 g per can. These cans were then topped with the foam shied and 1.35 mL metering head. Cans of Prototype B were placed at 5° C. and 25° C. for informal stability assessment. The table below provides the lab batch Lots for Prototype B.

TABLE 14
Prototype B Bulk formulation. Amounts are shown in grams.

| | Lot 1797-1109R03 | | Lot 1797-1110R01 | | Lot 1797-1110R02 | |
|---|---|---|---|---|---|---|
| | Placebo | | 30 μg/dose | | 3000 μg/dose | |
| Ingredient | % w/w | Amounts | % w/w | Amounts | % w/w | Amounts |
| Propylene Glycol USP/EP | 16.4800 | 230.73 | 16.4800 | 164.84 | 16.4800 | 115.36 |
| Cetyl Alcohol NF | 0.7000 | 9.80 | 0.7000 | 7.01 | 0.7000 | 4.90 |
| Emulsifying Wax NF | 1.4000 | 19.62 | 1.4000 | 14.00 | 1.4000 | 9.80 |
| Propylparaben NF | 0.0200 | 0.2830 | 0.0200 | 0.1998 | 0.0200 | 0.1418 |
| Polyoxylene (10) Stearyl | 1.4000 | 19.61 | 1.4000 | 14.03 | 1.4000 | 9.80 |
| Ether NF | | | | | | |
| Purified Water USP/EP | 79.5695 | 1113.97 | 79.5669 | 795.70 | 79.3092 | 556.99 |
| Sodium Dihydrogen | 0.1165 | 1.63 | 0.1165 | 1.17 | 0.1165 | 0.8160 |
| Phosphate Monohydrate | | | | | | |
| USP | | | | | | |
| Sodium Phosphate | 0.1640 | 2.30 | 0.1640 | 1.64 | 0.1640 | 1.15 |
| Dibasic USP | | | | | | |
| Disodium EDTA USP | 0.0500 | 0.7000 | 0.0500 | 0.4992 | 0.0500 | 0.3501 |
| Methylparaben NF | 0.1000 | 1.41 | 0.1000 | 1.01 | 0.1000 | 0.7007 |
| Peptide (adjusted for | NA | NA | 0.002603 | 0.0270 | 0.260300 | 1.82 |
| potency) | | | | | | |
| Total | 100.00 | 1400 | 100.00 | 1000 | 100.00 | 702 |

NA = not applicable.

### Example 17. Stability Results

The following tables provide the informal stability physical and analytical results for the lots produced for Prototypes A and B. Cans of Prototypes A and B were placed on informal stability at 5° C. and 25° C. and tested at the time points indicated.

TABLE 15
Lot 1797-1109R02 - Prototype A Placebo Physical Results.
Lot 1797-1109R02 - Prototype A Placebo Physical Results

| | | Initial | Initial | 1 Mo @ | 1 Mo @ | 2 Mo @ | 3 Mo @ | 3 Mo @ | 6 Mo @ | 6 Mo @ |
|---|---|---|---|---|---|---|---|---|---|---|
| | | (bulk) | (Foam) | 5° C. | 25° C. | 25° C. | 5° C. | 25° C. | 5° C. | 25° C. |
| Specific Gravity | | 0.983 | — | — | — | — | — | — | — | — |
| pH | 6.5-7.5 | 7.1 | 7.0 | 6.8 | 6.9 | 7.3 | 6.9 | 7.0 | 7.0 | 6.9 |
| Foam Height | Sample 1 | — | 20 | 26 | 19 | 17 | 28 | 29 | 25 | 24 |
| (mL) | Sample 2 | — | 29 | 28 | 28 | 11 | 26 | 25 | 26 | 27 |
| | Sample 3 | — | 24 | 27 | 25 | 22 | 26 | 23 | 24 | 26 |
| Duration of | Sample 1 | — | 1 | 1 | 1 | 1 | 1 | 1 | 1 | 1 |
| Expansion | Sample 2 | — | 1 | 1 | 1 | 1 | 1 | 1 | 1 | 1 |
| (min) | Sample 3 | — | 1 | 1.5 | 1 | 1 | 1 | 1 | 1 | 1 |
| Weight | Sample 1 | — | 1.18 | 1.51 | 1.27 | 1.15 | 1.34 | 1.19 | 1.28 | 1.17 |
| per | Sample 2 | — | 1.33 | 1.30 | 1.26 | 1.13 | 1.23 | 1.20 | 1.26 | 1.22 |
| Actuation | Sample 3 | — | 1.22 | 1.25 | 1.20 | 1.14 | 1.23 | 1.10 | 1.16 | 1.21 |
| (g) | Sample 4 | — | 1.16 | 1.23 | 1.20 | 0.95 | 1.21 | 1.11 | 1.16 | 1.20 |
| Appearance | Sample 1 | — | Pass | Pass | Pass | Pass | Pass | Pass | Pass | Pass |
| White | Sample 2 | — | Pass | Pass | Pass | Pass | Pass | Pass | Pass | Pass |
| Foam | Sample 3 | — | Pass | Pass | Pass | Pass | Pass | Pass | Pass | Pass |
| | Sample 4 | — | Pass | Pass | Pass | Pass | Pass | Pass | Pass | Pass |

TABLE 16
1797-1111R01- Prototype A 30 μm dose Physical Results.
Lot 1797-1109R02 - Prototype A Placebo Physical Results

| | | Initial | Initial | 1 Mo @ | 1 Mo @ | 2 Mo @ | 3 Mo @ | 3 Mo @ | 6 Mo @ | 6 Mo @ |
|---|---|---|---|---|---|---|---|---|---|---|
| | | (bulk) | (Foam) | 5° C. | 25° C. | 25° C. | 5° C. | 25° C. | 5° C. | 25° C. |
| Specific Gravity | | 0.983 | — | — | — | — | — | — | — | — |
| pH | 6.5-7.5 | 7.1 | 7.0 | 6.8 | 6.9 | 7.3 | 6.9 | 7.0 | 7.0 | 6.9 |
| Foam | Sample 1 | — | 20 | 26 | 19 | 17 | 28 | 29 | 25 | 24 |
| Height | Sample 2 | — | 29 | 28 | 28 | 11 | 26 | 25 | 26 | 27 |
| (mt) | Sample 3 | — | 24 | 27 | 25 | 22 | 26 | 23 | 24 | 26 |
| Duration of | Sample 1 | — | 1 | 1 | 1 | 1 | 1 | 1 | 1 | 1 |
| Expansion | Sample 2 | — | 1 | 1 | 1 | 1 | 1 | 1 | 1 | 1 |
| (min) | Sample 3 | — | 1 | 1.5 | 1 | 1 | 1 | 1 | 1 | 1 |
| Weight per | Sample 1 | — | 1.18 | 1.51 | 1.27 | 1.15 | 1.34 | 1.19 | 1.28 | 1.17 |
| Actuation | Sample 2 | — | 1.33 | 1.30 | 1.26 | 1.13 | 1.23 | 1.20 | 1.26 | 1.22 |
| (G) | Sample 3 | — | 1.22 | 1.25 | 1.20 | 1.14 | 1.23 | 1.10 | 1.16 | 1.21 |
| | Sample 4 | — | 1.16 | 1.23 | 1.20 | 0.95 | 1.21 | 1.11 | 1.16 | 1.20 |
| Appearance | Sample 1 | — | Pass | Pass | Pass | Pass | Pass | Pass | Pass | Pass |
| White | Sample 2 | — | Pass | Pass | Pass | Pass | Pass | Pass | Pass | Pass |
| Foam | Sample 3 | — | Pass | Pass | Pass | Pass | Pass | Pass | Pass | Pass |
| | Sample 4 | — | Pass | Pass | Pass | Pass | Pass | Pass | Pass | Pass |

TABLE 17
Lot 1797-1111R02 - Prototype A 3000 μg dose Physical Results
Lot 1797-1111R02 - Prototype A 3000 μg dose Physical Results

| | | Initial | Initial | 1 Mo @ | 1 Mo @ | 2 Mo @ | 3 Mo @ | 3 Mo @ | 6 Mo @ | 6 Mo @ |
|---|---|---|---|---|---|---|---|---|---|---|
| | | (bulk) | (Foam) | 5 C. | 25 C. | 25 C. | 5 C. | 25 C. | 5 C. | 25 C. |
| Specific Gravity | | 0.987 | — | — | — | — | — | — | — | — |
| pH | 6.5-7.5 | 6.8 | 6.9 | 6.8 | 6.8 | 7.1 | 6.9 | 6.9 | 6.8 | 6.9 |
| Foam | Sample 1 | — | 29 | 28 | 28 | 24 | 22 | 28 | 29 | 23 |
| Height | Sample 2 | — | 30 | 23 | 22 | 23 | 29 | 26 | 27 | 24 |
| (mL) | Sample 3 | — | 31 | 20 | 28 | 21 | 29 | 26 | 34 | 25 |
| Duration of | Sample 1 | — | 1 | 1 | 1 | 1 | 1 | 1 | 1 | 1 |
| Expansion | Sample 2 | — | 1.5 | 1 | 1 | 1 | 1 | 1 | 1 | 1 |
| (min) | Sample 3 | — | 1.25 | 1 | 1 | 1 | 1 | 1 | 1 | 1 |
| Weight per | Sample 1 | — | 1.38 | 1.24 | 1.51 | 1.18 | 1.14 | 1.34 | 1.31 | 1.14 |
| Actuation | Sample 2 | — | 1.34 | 1.27 | 1.30 | 1.20 | 1.32 | 1.23 | 1.24 | 1.12 |
| (g) | Sample 3 | — | 1.35 | 1.24 | 1.25 | 1.11 | 1.32 | 1.23 | 1.82 | 1.17 |
| | Sample 4 | — | 1.36 | 1.28 | 1.23 | 0.92 | 1.13 | 1.21 | 1.24 | 1.25 |
| Appearance | Sample 1 | — | Pass | Pass | Pass | Pass | Pass | Pass | Pass | Pass |
| White | Sample 2 | — | Pass | Pass | Pass | Pass | Pass | Pass | Pass | Pass |
| Foam | Sample 3 | — | Pass | Pass | Pass | Pass | Pass | Pass | Pass | Pass |
| | Sample 4 | — | Pass | Pass | Pass | Pass | Pass | Pass | Pass | Pass |

TABLE 18
Percent recovery in pharmaceutical rectal foam composition. Lot 1797-1109R02 - Prototype A Placebo.
% Recovery in pharmaceutical rectal foam composition. Lot 1797-1109R02 - Prototype A

| | | Bulk | TO | T1 | | T2 | T3 | | T6 | |
|---|---|---|---|---|---|---|---|---|---|---|
| | Location | — | — | 5 C. | 25 C. | 25° C. | 5 C. | 25 C. | 5 C. | 25 C. |
| Peptide | Beginning | — | — | — | — | — | — | — | — | — |
| (API) | Middle | — | — | — | — | — | — | — | — | — |
| | End | — | — | — | — | — | — | — | — | — |
| Mean | | — | — | — | — | — | — | — | — | — |
| % RSD | | — | — | — | — | — | — | — | — | — |
| Methylparaben | Beginning | — | — | 102.9 | 103.4 | 104.3 | 104.5 | 103.5 | 104.5 | 101.5 |
| | Middle | — | — | 101.7 | 100.4 | 103.3 | 101.2 | 101.1 | 103.8 | 102.3 |
| | End | — | — | 102.9 | 102.2 | 102.5 | 102.2 | 100.4 | 103.2 | 100/9 |
| Mean | | — | 103.3* | 102.5 | 102.0 | 103.3 | 102.6 | 101.7 | 103.8 | 101.6 |
| % RSD | | — | 0.31 | 0.68 | 1.48 | 0.79 | 1.68 | 1.57 | 0.6 | 0.71 |
| Propylparaben | Beginning | — | — | 102.8 | 103.9 | 105.3 | 103.7 | 103.3 | 104.4 | 102.5 |
| | Middle | — | — | 101.6 | 100.7 | 104.2 | 100.3 | 101.2 | 108.8 | 103.2 |
| | End | — | — | 103.0 | 102.8 | 103.5 | 101.5 | 100.4 | 103.1 | 101.8 |

| | | | | | | | | | |
|---|---|---|---|---|---|---|---|---|---|
| Mean | — | 103.5* | 102.5 | 102.5 | 104.3 | 101.9 | 101.6 | 103.8 | 102.5 |
| % RSD | — | −0.30 | 0.74 | 1.59 | 0.88 | 1.67 | 1.49 | 0.60 | 0.68 |

*Mean of n = 3

TABLE 19
Percent recovery in pharmaceutical rectal foam composition. Lot 1797-1109R02 - Prototype A 30 μg/mL.
% Recovery in pharmaceutical rectal foam composition. Lot 1797-1109R02 - Prototype A 30 μg/mL

| | | Bulk | TO | T1 | | T2 | T3 | | T6 | |
|---|---|---|---|---|---|---|---|---|---|---|
| | Location | — | — | 5° C. | 25° C. | 25° C. | 5° C. | 25° C. | 5° C. | 25° C. |
| Peptide | Beginning | — | — | 101.0 | 97.5 | 98.6 | 102.0 | 94.4 | 101.6 | 91.0 |
| (API) | Middle | — | — | 101.6 | 98.5 | 97.6 | 100.9 | 96.0 | 101.0 | 90.6 |
| | End | — | — | 98.5 | 93.3 | 96.9 | 101.3 | 93.3 | 101.2 | 89.7 |
| Mean | | 100.6* | 101.9** | 100.4 | 96.4 | 97.7 | 101.4 | 94.6 | 101.3 | 90.4 |
| % RSD | | 0.37 | 0.80 | 1.64 | 2.86 | 0.87 | 0.53 | 1.42 | 0.31 | 0.70 |
| Methylparaben | Beginning | — | — | 101.0 | 100.0 | 99.0 | 99.3 | 98.0 | 100.6 | 99.1 |
| | Middle | — | — | 100.3 | 100.6 | 98.7 | 98.7 | 97.6 | 99.8 | 98.3 |
| | End | — | — | 99.4 | 99.0 | 97.1 | 98.6 | 98.5 | 99.6 | 97.2 |
| Mean | | 100.9* | 98.0** | 100.2 | 99.9 | 98.3 | 98.9 | 98.1 | 100.0 | 98.2 |
| % RSD | | 0.34 | 1.42 | 0.80 | 0.81 | 1.1 | 0.37 | 0.46 | 0.53 | 0.99 |
| Propylparaben | Beginning | — | — | 101.6 | 100.8 | 100.5 | 99.0 | 98.5 | 100.9 | 100.5 |
| | Middle | — | — | 100.8 | 101.3 | 100.5 | 98.6 | 98.1 | 100.2 | 99.9 |
| | End | — | — | 100.2 | 100.0 | 98.7 | 98.6 | 99.2 | 100.1 | 98.8 |

| | | | | | | | | | |
|---|---|---|---|---|---|---|---|---|---|
| Mean | 100.9* | 98.9** | 100.9 | 100.7 | 99.9 | 98.8 | 98.6 | 100.4 | 99.7 |
| % RSD | 0.39 | 1.41 | 0.70 | 0.65 | 1.1 | 0.24 | 0.54 | 0.47 | 0.87 |

*Mean of n = 6
**Mean of n = 10
Mean % Recovery at each time points is used.

TABLE 20
Percent recovery in pharmaceutical rectal foam composition. Lot 1797-1109R02 - Prototype A 3000 μg/mL.
% Recovery in pharmaceutical rectal foam composition. Lot 1797-1109R02 - Prototype A 3000 μg/mL

| | | Bulk | T0 | T1 | | T2 | T3 | | T6 | |
|---|---|---|---|---|---|---|---|---|---|---|
| | Location | — | — | 5° C. | 25° C. | 25° C. | 5° C. | 25° C. | 5° C. | 25° C. |
| Peptide (API) | Beginning | — | — | 98.3 | 93.8 | 89.6 | 99.8 | 87.7 | 97.9 | 83.3 |
| | Middle | — | — | 96.6 | 93.3 | 90.2 | 97.3 | 86_5 | 96.6 | 83.4 |
| | End | — | — | 97.3 | 93.4 | 90.1 | 97.9 | 843 | 96.0 | 83.4 |
| Mean | | 92.2* | 97.5** | 97.4 | 93.5 | 90.0 | 98.3 | 86.3 | 96.9 | 83.4 |
| % RSD | | 0.41 | 1.53 | 0.88 | 0.28 | 0.37 | 1.29 | 1.76 | 1.00 | 0.06 |
| Methylparaben | Beginning | — | — | 100.5 | 99.0 | 97.4 | 101.6 | 98_0 | 99.1 | 94.9 |
| | Middle | — | — | 99.2 | 98.1 | 97.7 | 102.0 | 973 | 99.0 | 96.6 |
| | End | — | — | 98.7 | 97.7 | 98.6 | 100.5 | 96.9 | 98.3 | 95.7 |
| Mean | | 98.5* | 99.6** | 99.5 | 98.3 | 97.9 | 101.4 | 97.4 | 98.8 | 95.7 |
| % RSD | | 0.27 | 0.75 | 0.93 | 0.68 | 0.65 | 0.75 | 0.58 | 0.45 | 0.87 |
| Propylparaben | Beginning | — | — | 101.1 | 99.5 | 98.9 | 101.4 | 98.2 | 99.3 | 97.7 |
| | Middle | — | — | 99.6 | 98.6 | 99.1 | 101.8 | 97.4 | 99.2 | 99.6 |
| | End | — | — | 99.2 | 98.5 | 100.1 | 100.5 | 973 | 98.6 | 98.7 |

| | | | | | | | | | |
|---|---|---|---|---|---|---|---|---|---|
| Mean | 98.5* | 100.4** | 100.0 | 98.9 | 99.4 | 101.2 | 97.6 | 99.0 | 98.7 |
| % RSD | 0.25 | 0.79 | 1.00 | 0.56 | 0.63 | 0.66 | 0.54 | 0.38 | 0.94 |

*Mean of n = 6
**Mean of n = 10
Mean % Recovery at each time points is used.

TABLE 21
Lot 1797-1109R03 - Prototype B Placebo Physical Results
Lot 1797-1109R03 - Prototype B Placebo Physical Results

| | | Initial | Initial | 1 Mo @ | 1 Mo @ | 2 Mo @ | 3 Mo @ | 3 Mo @ | 6 Mo @ | 6 Mo @ |
|---|---|---|---|---|---|---|---|---|---|---|
| | | (bulk) | (Foam) | 5° C. | 25° C. | 25° C. | 5° C. | 25° C. | 5° C. | 25° C. |
| Specific Gravity | | 1.015 | — | — | — | — | — | — | — | — |
| pH | 6.5-7.5 | 7.1 | 7.1 | 7.1 | 6.7 | 7.3 | 7.1 | 6.9 | 6.9 | 6.8 |
| Foam | Sample 1 | — | 16 | 15 | 13 | 7 | 11 | 13 | 12 | 14 |
| Height | Sample 2 | — | 14 | 5 | 13 | 21 | 16 | 15 | 20 | 15 |
| (mL) | Sample 3 | — | 17 | 17 | 13 | 21 | 15 | 14 | 19 | 14 |
| Duration of | Sample 1 | — | 0.5 | 0.25 | 0.25 | 0.5 | 0.25 | 0.25 | 0.25 | 0.25 |
| Expansion | Sample 2 | — | 0.5 | 0.25 | 0.25 | 0.5 | 0.25 | 0.25 | 0.25 | 0.25 |
| (min) | Sample 3 | — | 0.5 | 0.25 | 0.25 | 0.5 | 0.25 | 0.25 | 0.25 | 0.25 |
| Weight per | Sample 1 | — | 1.33 | 1.14 | 1.23 | 1.18 | 1.19 | 1.27 | 1.24 | 1.20 |
| Actuation | Sample 2 | — | 1.27 | 1.30 | 1.25 | 1.08 | 1.34 | 1.25 | 1.32 | 1.63 |
| (g) | Sample 3 | — | 1.34 | 2.33 | 1.26 | 1.04 | 1.32 | 1.23 | 1.30 | 1.17 |
| | Sample 4 | — | 1.24 | 1.35 | 1.10 | 0.98 | 1.31 | 1.16 | 1.29 | 0.89 |
| Appearance | Sample 1 | — | Pass | Pass | Pass | Pass | Pass | Pass | Pass | Pass |
| White | Sample 2 | — | Pass | Pass | Pass | Pass | Pass | Pass | Pass | Pass |
| Foam | Sample 3 | — | Pass | Pass | Pass | Pass | Pass | Pass | Pass | Pass |
| | Sample 4 | — | Pass | Pass | Pass | Pass | Pass | Pass | Pass | Pass |

TABLE 22
Lot 1797-1110R01 - Prototype B 30 μg dose Physical Results.
Lot 1797-1110R01 - Prototype B 30 μg dose Physical Results

| | | Initial | Initial | 1 Mo @ | 1 Mo @ | 2 Mo @ | 3 Mo @ | 3 Mo @ | 6 Mo @ | 6 Mo @ |
|---|---|---|---|---|---|---|---|---|---|---|
| | | (bulk) | (Foam) | 5° C. | 25° C. | 25° C. | 5° C. | 25° C. | 5° C. | 25° C. |
| Specific Gravity | | 1.005 | — | — | — | — | — | — | — | — |
| pH | 6.5-7.5 | 7.1 | 7.1 | 7.1 | 7.1 | 7.3 | 7.0 | 7.0 | 6.9 | 6.8 |
| Foam | Sample 1 | — | 10 | 12 | 11 | 8 | 12 | 12 | 18 | 24 |
| Height | Sample 2 | — | 11 | 12 | 11 | 14 | 14 | 15 | 18 | 15 |
| (mL) | Sample 3 | — | 13 | 5 | 10 | 5 | 15 | 16 | 18 | — |
| Duration of | Sample 1 | — | 0.5 | 0.25 | 0.25 | 0.5 | 0.25 | 0.25 | 0.25 | 0.25 |
| Expansion | Sample 2 | — | 0.5 | 0.25 | 0.25 | 0.5 | 0.25 | 0.25 | 0.25 | 0.25 |
| (min) | Sample 3 | — | 0.5 | 0.25 | 0.25 | 0.5 | 0.25 | 0.25 | 0.25 | — |
| Weight per | Sample 1 | — | 1.27 | 1.22 | 1.21 | 1.05 | 1.14 | 1.11 | 1.26 | 1.85 |
| Actuation | Sample 2 | — | 1.29 | 1.20 | 1.33 | 1.11 | 1.30 | 1.21 | 1.26 | 0.96 |
| (g) | Sample 3 | — | 1.36 | 1.30 | 1.23 | 0.99 | 1.27 | 1.24 | 1.29 | — |
| | Sample 4 | — | 1.28 | 1.35 | 1.19 | 0.96 | 1.30 | 1.16 | 0.93 | — |
| Appearance | Sample 1 | — | Pass | Pass | Pass | Pass | Pass | Pass | Pass | Pass |
| White | Sample 2 | — | Pass | Pass | Pass | Pass | Pass | Pass | Pass | Pass |
| Foam | Sample 3 | — | Pass | Pass | Pass | Pass | Pass | Pass | Pass | — |
| | Sample 4 | — | Pass | Pass | Pass | Pass | Pass | Pass | Pass | — |

TABLE 23
Lot 1797-1110R02 - Prototype B 3000 μg dose Physical Results.
Lot 1797-1110R02 - Prototype B 3000 μg dose Physical Results

| | | Initial | Initial | 1 Mo @ | 1 Mo @ | 2 Mo @ | 3 Mo @ | 3 Mo @ | 6 Mo @ | 6 Mo @ |
|---|---|---|---|---|---|---|---|---|---|---|
| | | (bulk) | (Foam) | 5° C. | 25° C. | 25° C. | 5° C. | 25° C. | 5° C. | 25° C. |
| Specific Gravity | | 1.01 | — | — | — | — | — | — | — | — |
| pH | 6.5-7.5 | 6.9 | 7.0 | 7.0 | 6.9 | 7.1 | 6.8 | 6.9 | 6.8 | 6.7 |
| Foam | Sample 1 | — | 15 | 8 | 18 | 20 | 11 | 15 | 16 | 14 |
| Height | Sample 2 | — | 16 | 16 | 17 | 10 | 14 | 19 | 19 | 15 |
| (mL) | Sample 3 | — | 16 | 15 | 7 | 14 | 15 | 19 | 18 | 13 |
| Duration of | Sample 1 | — | 0.5 | 0.25 | 0.25 | 0.5 | 0.25 | 0.25 | 0.25 | 0.25 |
| Expansion | Sample 2 | — | 0.25 | 0.25 | 0.25 | 0.5 | 0.25 | 0.25 | 0.25 | 0.25 |
| (min) | Sample 3 | — | 0.25 | 0.25 | 0.25 | 0.5 | 0.25 | 0.25 | 0.25 | 0.25 |
| Weight per | Sample 1 | — | 1.26 | 1.15 | 1.21 | 1.11 | 1.16 | 1.31 | 1.22 | 1.14 |
| Actuation | Sample 2 | — | 1.23 | 1.19 | 2.37 | 1.09 | 1.20 | 1.59 | 1.26 | 1.10 |
| | Sample 3 | — | 1.23 | — | 1.12 | 1.01 | 1.19 | 1.33 | 1.17 | 0.94 |
| | Sample 4 | — | 1.18 | 1.31 | 1.05 | 1.02 | 1.23 | 1.20 | 0.85 | 1.01 |
| Appearance | Sample 1 | — | Pass | Pass | Pass | Pass | Pass | Pass | Pass | Pass |
| White | Sample 2 | — | Pass | Pass | Pass | Pass | Pass | Pass | Pass | Pass |
| Foam | Sample 3 | — | Pass | Pass | Pass | Pass | Pass | Pass | Pass | Pass |
| | Sample 4 | — | Pass | Pass | Pass | Pass | Pass | Pass | Pass | Pass |

TABLE 24
Percent recovery in pharmaceutical rectal foam composition. Lot 1797-1109R03 - Prototype B Placebo.
% Recovery in pharmaceutical rectal foam composition. Lot 1797-1109R03 - Prototype B Placebo

| | | Bulk | TO | T1 | | T2 | T3 | | T6 | |
|---|---|---|---|---|---|---|---|---|---|---|
| | Location | — | — | 5° C. | 25° C. | 25° C. | 5° C. | 25° C. | 5° C. | 25° C. |
| Peptide | Beginning | — | — | — | — | — | — | — | — | — |
| (API) | Middle | — | — | — | — | — | — | — | — | — |
| | End | — | — | — | — | — | — | — | — | — |
| Mean | | — | — | — | — | — | — | — | — | — |
| % RSD | | — | — | — | — | — | — | — | — | — |
| | Beginning | — | — | 96.5 | 103.6 | 98.6 | 98.5 | 97.9 | 98.9 | 96.5 |
| Methylparaben | Middle | — | — | 99.6 | 98.6 | 99.3 | 98.5 | 98.8 | 97.8 | 95.2 |
| | End | — | — | 104.8 | 98.9 | 97.3 | 98.9 | 98.4 | 100.2 | 96.5 |
| Mean | | — | 98.8* | 100.3 | 100.4 | 98.4 | 98.6 | 98.4 | 99.0 | 96.0 |
| % RSD | | — | 0.54 | 4.18 | 2.79 | 0.94 | 0.22 | 0.46 | 1.12 | 0.72 |
| | Beginning | — | | 95.4 | 102.7 | 98.7 | 97.0 | 98.6 | 99.4 | 97.1 |
| Propylparaben | Middle | — | | 98.7 | 98.0 | 99.3 | 97.0 | 99.1 | 96.3 | 96.0 |
| | End | — | | 103.4 | 98.7 | 98.2 | 97.5 | 98.9 | 97.6 | 97.7 |

| | | | | | | | | | |
|---|---|---|---|---|---|---|---|---|---|
| Mean | — | 9.85* | 99.2 | 99.8 | 98.7 | 97.2 | 98.8 | 97.7 | 96.9 |
| % RSD | — | 0.54 | 4.05 | 2.54 | 0.57 | 0.27 | 0.29 | 1.58 | 0.92 |

*Mean of n = 3
Mean % Recovery at each time point is used.

TABLE 25
Percent recovery in pharmaceutical rectal foam composition. Lot 1797-1110R01 - Prototype B 30 μm/gL.
% Recovery in pharmaceutical rectal foam composition. Lot 1797-1110R01 - Prototype B 30 μg/mL

| | | Bulk | TO | T1 | | T2 | T3 | | T6 | |
|---|---|---|---|---|---|---|---|---|---|---|
| | Location | — | — | 5° C. | 25° C. | 25° C. | 5° C. | 25° C. | 5° C. | 25° C. |
| Peptide | Beginning | — | — | 100.2 | 97.3 | 97.3 | 102.1 | 95.3 | 101.8 | 90.6 |
| (API) | Middle | — | — | 99.5 | 96.2 | 98.9 | 102.3 | 96.3 | 101.8 | 91.2 |
| | End | — | — | 99.6 | 9.64 | 98.2 | 101.9 | 95.2 | 101.5 | 91.5 |
| Mean | | 99.6* | 100.1** | 99.8 | 96.6 | 98.1 | 102.1 | 95.6 | 101.7 | 91.1 |
| % RSD | | 0.5 | 0.5 | 0.38 | 0.61 | 0.85 | 0.19 | 0.64 | 0.19 | 0.51 |
| | Beginning | — | — | 98.9 | 91.5 | 98.4 | 97.5 | 98.1 | 98.3 | 95.7 |
| Methylparaben | Middle | — | — | 100.4 | 98.2 | 98.5 | 98.8 | 97.8 | 97.1 | 96.1 |
| | End | — | — | 99.2 | 98.7 | 96.9 | 98.1 | 96.8 | 96.8 | 96.6 |
| Mean | | 101.1* | 99.8** | 99.5 | 98.1 | 97.9 | 98.1 | 97.6 | 97.4 | 96.1 |
| % RSD | | 0.29 | 1.31 | 0.80 | 0.61 | 0.89 | 0.64 | 0.67 | 0.81 | 0.44 |
| | Beginning | — | — | 96.1 | 95.8 | 97.4 | 94.1 | 96.6 | 95.4 | 95.1 |
| Propylparaben | Middle | — | — | 97.8 | 96.1 | 97.0 | 96.0 | 95.8 | 94.3 | 95.3 |
| | End | — | — | 96.3 | 96.7 | 96.3 | 94.9 | 95.5 | 94.0 | 96.2 |

| | | | | | | | | | |
|---|---|---|---|---|---|---|---|---|---|
| Mean | 99.0* | 97.6** | 96.7 | 96.2 | 96.9 | 95.0 | 95.9 | 94.5 | 95.5 |
| % RSD | 0.40 | 1.17 | 0.96 | 0.48 | 0.57 | 0.97 | 0.60 | 0.78 | 0.57 |

*Mean of n = 3
**Mean of n = 10
Mean % Recovery at each time point is used.

TABLE 26
Percent recovery in pharmaceutical rectal foam composition. Lot 1797-1110R02 - Prototype B 3000 μg/mL.
% Recovery in pharmaceutical rectal foam composition. Lot 1797-1110R02 - Prototype B 3000 μg/mL

| | | Bulk | TO | T1 | | T2 | T3 | | T6 | |
|---|---|---|---|---|---|---|---|---|---|---|
| | Location | — | — | 5° C. | 25° C. | 25° C. | 5° C. | 25° C. | 5° C. | 25° C. |
| Peptide | Beginning | — | — | 101.7 | 99.0 | 97.5 | 102.6 | 91.4 | 1.02.6 | 86.1 |
| (API) | Middle | — | — | 101.0 | 97.8 | 94.6 | 99.6 | 90.0 | 102.7 | 86.5 |
| | End | — | — | 102.1 | 98.0 | 95.6 | 100.8 | 90.0 | 102.8 | 86.8 |
| Mean | | 98.9* | 100.2** | 101.6 | 98.3 | 95.9 | 101.0 | 90.5 | 102.7 | 86.5 |
| % RSD | | 0.97 | 0.67 | 0.55 | 0.65 | 1.54 | 1.49 | 0.93 | 0.11 | 0.42 |
| Methylparaben | Beginning | — | — | 95.5 | 94.4 | 97.6 | 97.0 | 95.2 | 95.8 | 95.2 |
| | Middle | — | — | 97.5 | 95.6 | 95.3 | 96.3 | 95.4 | 96.5 | 94.3 |
| | End | — | — | 97.2 | 95.6 | 95.3 | 95.7 | 93.1 | 97.1 | 94.7 |
| Mean | | 98.3* | 97.7** | 96.7 | 95.2 | 96.1 | 96.4 | 95.2 | 96.4 | 94.7 |
| % RSD | | 0.32 | 0.53 | 1.12 | 0.73 | 1.39 | 0.66 | 0.17 | 0.68 | 0.49 |
| Propylparaben | Beginning | — | — | 97.3 | 95.9 | 100.1 | 96.9 | 96.4 | 96.8 | 97.4 |
| | Middle | — | — | 99.1 | 96.8 | 97.6 | 96.0 | 96.6 | 97.2 | 96.6 |
| | End | — | — | 98.5 | 97.0 | 97.4 | 95.9 | 96.5 | 97.9 | 96.8 |

| | | | | | | | | | |
|---|---|---|---|---|---|---|---|---|---|
| Mean | 98.6* | 99.3** | 98.3 | 96.6 | 98.4 | 96.3 | 96.5 | 97.2 | 96.9 |
| % RSD | 0.26 | 0.72 | 0.93 | 0.61 | 1.55 | 0.59 | 0.10 | 0.67 | 0.40 |

*Mean of n = 3
**Mean of n = 10
Mean % Recovery at each time point is used.

For Prototype A, the informal stability for Placebo, 30 μg and 3000 μg were consistent for the physical parameters evaluated throughout the study. It was observed that Prototype A produced a stable foam upon actuation. For Prototype A, the analytical results for Active and Preservatives in the Placebo, 30 μg, and 3000 μg were steady throughout the study. The only observation is that at 25° C. the API concentration dropped in the 30 μg and 3000 μg samples over the course of the study.

For Prototype B, the informal stability for Placebo, 30 μg, and 3000 μg were reliable for the physical parameters evaluated throughout the study. The only observation, while not runny or fluid, was that the foam for Prototype B collapsed rather quickly after actuation. For Prototype B, the analytical results for Active and Preservatives in the Placebo, 30 μg, and 3000 μg were steady throughout the study. In Prototype B as well, at 25° C. the API concentration dropped in the 30 μg and 3000 μg samples over the course of the study.

### Example 18. Formula Enhancement

Based on the performance on informal stability example above, Prototype A was selected for further evaluation. One concern was the actuation amount dispensed, and determining an acceptable range thereof. Further enhancements to Prototype A were performed using the Placebo formulation. In addition, other propellants were re-evaluated with Prototype A. The table below provides a summary of the additional propellants evaluated. The table below provides the propellant name, the PSI specification range for the propellant and the composition and ratios in these propellants. Tables 30 through 36 provides the raw data generated during this study.

TABLE 27
Propellants evaluated for the pharmaceutical rectal foam composition.

| Propellant | PSI | Composition(s)/ratios |
|---|---|---|
| DME | 49-55 | Dimethyl Ether/n-Butane (53/47) |
| A17 | 15-19 | n-Butane (>97) |
| A31 | 29-33 | Isobutane (>95) |
| AP35 | 33-37 | Isobutane (83-89)/n-Butane(5-9)/Propane(5-9) |
| A46 | 44-48 | Isobutane (76.6-88)/Propane (12-21.9) |
| A48 | 46-50 | n-Butane (30-60)/Isobutane (15-30)/Propane (20-45) |
| A70 | 68-72 | Isobutane (46-64)/Propane (35.7-52.3)/n-Butane (0-3) |
| AP70 | 68-72 | Propane (49.8-59.8)/n-Butane (22.3-32.3)/Isobutane(12.9-22.9) |

Bulk product was packaged in a 30-g aluminum aerosol cans with a metered dose valve. Each can contained 31 g of bulk and gassed with approximately 3.0 g (2.8 g-3.2 g) of each propellant. Based on the data in the actuation results (see below), the propellants DME and AP35 were selected for additional studies. Ten cans of Prototype A Placebo, each with either DME or AP35, along with previously actuated cans with AP70 were compared (see tables below). From these results, the propellant AP35 was chosen as providing a more consistent actuation amount.

Additionally, the Active concentrations were further revised from the initial 30 μg/dose and 3000 μg/dose to the final concentrations 100 μg/dose and 300 μg/dose. With the decision to use AP35 propellant, the revised base formulation for the pharmaceutical rectal foam composition is shown in the table below.

TABLE 28
Revised base formulation for the pharmaceutical rectal foam composition.

| | Placebo | 100 μg | 300 μg |
|---|---|---|---|
| Ingredient | % w/w | % w/w | % w/w |
| Purified Water USP/EP | 77.5495 | 77.5399 | 77.5207 |
| Propylene Glycol USP | 10.0000 | 10.0000 | 10.0000 |
| Sodium Phosphate Dibasic USP | 0.1640 | 0.1640 | 0.1640 |
| Sodium Dihydrogen Phosphate Monohydrate USP | 0.1165 | 0.1165 | 0.1165 |
| Disodium EDTA USP | 0.0500 | 0.0500 | 0.0500 |
| Methylparaben NF | 0.1000 | 0.1000 | 0.1000 |
| Light Mineral Oil NF | 6.0000 | 6.0000 | 6.0000 |
| Isopropyl Myristate NF | 0.5000 | 0.5000 | 0.5000 |
| White Petrolatum USP | 1.0000 | 1.0000 | 1.0000 |
| Polyoxyl 20 Cetostearyl Ether NF | 2.5000 | 2.5000 | 2.5000 |
| Cetyl Alcohol NF | 1.0000 | 1.0000 | 1.0000 |
| Stearyl Alcohol NF | 1.0000 | 1.0000 | 1.0000 |
| Propylparaben NF | 0.0200 | 0.0200 | 0.0200 |
| Peptide API (adjusted for potency) | 0.00000 | 0.0096085 | 0.028825 |
| Total | 100.00 | 100.00 | 100.00 |

Additional lab batches for the pharmaceutical rectal foam composition were produced for Microbial Limits Assay Validation. The Placebo, 100 μg/dose and 300 μg/dose batches produced based on the formula in the table above (see Example below).

TABLE 29
Proposed specifications pharmaceutical rectal foam composition.

| Specification | Bulk Product | Finished Product |
|---|---|---|
| Appearance | White to off-white thin, | White to off-white foam |
| | liquid emulsion | |
| Specific gravity | Report Results | — |
| pH | 6.5-7.5 (neat) | 6.5-7.5 (1 actuation dissolved in 100 g of |
| | | 5% Methanol/95% Water Solution |
| Peptide API | 90.0-110.0% L | 90.0-110.0% L (Alert limit: 95.0-105.0% |
| | | L) |
| Methylparaben | 90.0-110.0% F | 90.0-110.0% F |
| Propylparaben | 90.0-110.0% F | 90.0-110.0% F |
| Foam Height (mL) | — | NLT 20 mL |
| Weight per Actuation | — | 1.16 g ± 15% |

### CONCLUSION

The foregoing examples addressed the informal stability and evaluation for the pharmaceutical rectal foam composition Prototypes A and B. Based on the informal stability results, Prototype A was chosen for continued evaluation. Formulation enhancements to Prototype A provided that AP35 was a more ideal propellant for the product. The product was further refined to provide the concentrations of 100 μg/dose and 300 μg/dose in a 30-g aluminum aerosol can with a metered dose valve. Microbial Limit validation was performed on the revised dosage formulae for Prototype A.

TABLE 30
Raw data for Prototype A Placebo - actuation results.
PROTOTYPE A Placebo - actuation results for the following propellants

| | DME - 31 g/2.8 g | | | A17 - 31 g/1.6 g | | | A31 - 31 g/2.0 g | | | AP35 - 31 g/2.8 g | | | A46 - 31 g/2.3 g | | | A48 - 31 g/1.9 g | | | A70 - 31 g/2.7 g | | |
|---|---|---|---|---|---|---|---|---|---|---|---|---|---|---|---|---|---|---|---|---|---|
| Act. | | CW | | | CW | | | CW | | | CW | | | CW | | | CW | | | CW | |
| # | CW | dif. | FH | CW | dif. | FH | CW | dif. | FH | CW | dif. | FH | CW | dif. | FH | CW | dif. | FH | CW | dif. | FH |
| 0 | 54.42 | — | — | 53.49 | — | — | 53.85 | — | — | 54.66 | — | — | 53.91 | — | — | 53.59 | — | — | 54.44 | — | — |
| 1 | 53.22 | 1.20 | 26 | 52.38 | 1.11 | 22 | 52.59 | 1.26 | 27 | 53.47 | 1.19 | 30 | 52.85 | 1.06 | 30 | 52.39 | 1.20 | 24 | 53.24 | 1.20 | 35 |
| 2 | 51.93 | 1.29 | — | 51.07 | 1.31 | — | 51.33 | 1.26 | — | 52.18 | 1.29 | — | 51.59 | 1.26 | — | 51.19 | 1.20 | — | 52.00 | 1.24 | — |
| 3 | 50.65 | 1.28 | — | 49.82 | 1.25 | — | 50.09 | 1.24 | — | 50.92 | 1.26 | — | 50.34 | 1.25 | — | 50.04 | 1.15 | — | 50.76 | 1.24 | — |
| 4 | 49.47 | 1.18 | — | 48.62 | 1.20 | — | 48.88 | 1.21 | — | 49.70 | 1.22 | — | 49.14 | 1.20 | — | 48.92 | 1.12 | — | 49.58 | 1.18 | — |
| 5 | 48.30 | 1.17 | 27 | 47.41 | 1.21 | 17 | 47.72 | 1.16 | 21 | 48.54 | 1.16 | 26 | 47.98 | 1.16 | 23 | 47.85 | 1.07 | 19 | 48.50 | 1.08 | 30 |
| 6 | 47.16 | 1.14 | — | 46.3 | 1.11 | — | 46.60 | 1.12 | — | 47.43 | 1.11 | — | 46.86 | 1.12 | — | 46.79 | 1.06 | — | 47.35 | 1.15 | — |
| 7 | 46.11 | 1.05 | — | 45.24 | 1.06 | — | 45.55 | 1.05 | — | 46.37 | 1.06 | — | 45.79 | 1.07 | — | 45.80 | 0.99 | — | 46.30 | 1.05 | — |
| 8 | 45.06 | 1.05 | — | 44.24 | 1.00 | — | 44.49 | 1.06 | — | 45.28 | 1.09 | — | 44.78 | 1.01 | — | 44.83 | 0.97 | — | 45.27 | 1.03 | — |
| 9 | 44.02 | 1.04 | 25 | 43.28 | 0.96 | 13 | 43.5 | 0.99 | 17 | 44.24 | 1.04 | 22 | 43.75 | 1.03 | 19 | 43.92 | 0.91 | 16 | 44.11 | 1.16 | 30 |
| 10 | 42.96 | 1.06 | — | 42.33 | 0.95 | — | 42.52 | 0.98 | — | 43.19 | 1.05 | — | 42.78 | 0.97 | — | 42.99 | 0.93 | — | 43.12 | 0.99 | — |
| 11 | 41.90 | 1.06 | — | 41.41 | 0.92 | — | 41.56 | 0.96 | — | 42.14 | 1.05 | — | 41.80 | 0.98 | — | 42.08 | 0.91 | — | 42.14 | 0.98 | — |
| 12 | 40.87 | 1.03 | — | 40.53 | 0.88 | — | 40.62 | 0.94 | — | 41.10 | 1.04 | — | 40.86 | 0.94 | — | 41.20 | 0.88 | — | 41.13 | 1.01 | — |
| 13 | 39.74 | 1.13 | 27 | 39.67 | 0.86 | 12 | 39.71 | 0.91 | 15 | 40.04 | 1.06 | 22 | 39.96 | 0.90 | 16 | 40.36 | 0.84 | 13 | 40.15 | 0.98 | 24 |
| 14 | 38.66 | 1.08 | — | 38.81 | 0.86 | — | 38.76 | 0.95 | — | 38.98 | 1.06 | — | 39.05 | 0.91 | — | 39.50 | 0.86 | — | 39.12 | 1.03 | — |
| 15 | 37.57 | 1.09 | — | 37.96 | 0.85 | — | 37.84 | 0.92 | — | 37.89 | 1.09 | — | 38.15 | 0.90 | — | 38.62 | 0.88 | — | 38.11 | 1.01 | — |
| 16 | 36.49 | 1.08 | — | 37.15 | 0.81 | — | 36.95 | 0.89 | — | 36.74 | 1.15 | — | 37.25 | 0.90 | — | 37.74 | 0.88 | — | 37.10 | 1.01 | — |
| 17 | 35.44 | 1.05 | 24 | 36.37 | 0.78 | 9 | 36.03 | 0.92 | 15 | 35.62 | 1.12 | 23 | 36.27 | 0.98 | 17 | 36.90 | 0.84 | 12 | 36.07 | 1.03 | 29 |
| 18 | 34.33 | 1.11 | — | 35.58 | 0.79 | — | 35.12 | 0.91 | — | 34.50 | 1.12 | — | 35.33 | 0.94 | — | 36.01 | 0.89 | — | 35.01 | 1.06 | — |
| 19 | 33.23 | 1.10 | — | 34.51 | 1.07 | — | 34.21 | 0.91 | — | 33.33 | 1.17 | — | 34.44 | 0.89 | — | 35.11 | 0.90 | — | 33.96 | 1.05 | — |
| 20 | 32.13 | 1.10 | — | 33.77 | 0.74 | — | 33.30 | 0.91 | — | 32.15 | 1.18 | — | 33.53 | 0.91 | — | 34.21 | 0.90 | — | 32.92 | 1.04 | — |
| 21 | 30.92 | 1.21 | 26 | 33.06 | 0.71 | 7 | 32.40 | 0.90 | 15 | 30.98 | 1.17 | 26 | 32.58 | 0.95 | 16 | 33.28 | 0.93 | 14 | 31.80 | 1.12 | 25 |
| 22 | | | | | | | | | | | | | | | | | | | | | |

| | | | | | | |
|---|---|---|---|---|---|---|
| Mean =1.12 | Mean = 0.97 | Mean = 1.02 | Mean = 1.13 | Mean = 1.02 | Mean = 0.97 | Mean = 1.0 |
| % RSD = 6.8 | % RSD = 18.2 | % RSD = 12.9 | % RSD = 6.5 | % RSD = 11.7 | % RSD = 12.1 | % RSD = 7.8 |

“Act.” = actuation. “CW” = can weight in grams. “FH” = foam height in mL. “Dif.” = difference.

TABLE 31
Raw data for Prototype A Placebo - actuation results.
PROTOTYPE A Placebo (Set 1 thru 5)

| | DME - 31 g/2.8 g | | | DME 31 g/3.4 g | | | DME 31/2.8 g | | | DME 31 g/2.9 g | | | DME 31 g/2.4 g | | |
|---|---|---|---|---|---|---|---|---|---|---|---|---|---|---|---|
| Act. | | CW | | | CW | | | CW | | | CW | | | CW | |
| # | CW | dif. | FH | CW | dif. | FH | CW | dif. | FH | CW | dif. | FH | CW | dif. | FH |
| 0 | 54.42 | — | — | 55.07 | — | — | 54.42 | — | — | 54.72 | — | — | 54.10 | — | — |
| 1 | 53.22 | 1.20 | 26 | 53.83 | 1.24 | 35 | 53.16 | 1.26 | 31 | 53.49 | 1.23 | 22 | 52.75 | 1.35 | 25 |
| 2 | 51.93 | 1.29 | — | 52.56 | 1.27 | — | 51.96 | 1.20 | — | 52.19 | 1.30 | — | 51.41 | 1.34 | — |
| 3 | 50.65 | 1.28 | — | 51.36 | 1.20 | — | 50.83 | 1.13 | — | 50.88 | 1.31 | — | 50.14 | 1.27 | — |
| 4 | 49.47 | 1.18 | — | 50.23 | 1.13 | — | 49.72 | 1.11 | — | 49.64 | 1.24 | — | 48.94 | 1.20 | — |
| 5 | 48.30 | 1.17 | 27 | 49.14 | 1.09 | 31 | 48.64 | 1.08 | 25 | 48.44 | 1.20 | 31 | 47.77 | 1.17 | 25 |
| 6 | 47.16 | 1.14 | — | 48.04 | 1.10 | — | 47.55 | 1.09 | — | 47.34 | 1.10 | — | 46.68 | 1.09 | — |
| 7 | 46.11 | 1.05 | — | 46.96 | 1.08 | — | 46.51 | 1.04 | — | 46.32 | 1.02 | — | 45.63 | 1.05 | — |
| 8 | 45.06 | 1.05 | — | 45.90 | 1.06 | — | 45.45 | 1.06 | — | 45.30 | 1.02 | — | 44.62 | 1.01 | — |
| 9 | 44.02 | 1.04 | 25 | 44.81 | 1.09 | 29 | 44.39 | 1.06 | 23 | 44.20 | 1.10 | 29 | 43.64 | 0.98 | 20 |
| 10 | 42.96 | 1.06 | — | 43.70 | 1.11 | — | 43.34 | 1.05 | — | 43.19 | 1.01 | — | 42.89 | 0.95 | — |
| 11 | 41.90 | 1.06 | — | 42.58 | 1.12 | — | 42.28 | 1.06 | — | 42.18 | 1.01 | — | 41.72 | 0.97 | — |
| 12 | 40.87 | 1.03 | — | 41.46 | 1.12 | — | 41.23 | 1.05 | — | 41.20 | 0.98 | — | 40.81 | 0.91 | — |
| 13 | 39.74 | 1.13 | 27 | 40.33 | 1.13 | 30 | 40.14 | 1.09 | 23 | 40.04 | 1.16 | 29 | 39.87 | 0.94 | 19 |
| 14 | 38.66 | 1.08 | — | 39.19 | 1.14 | — | 39.03 | 1.11 | — | 39.00 | 1.04 | — | 38.93 | 0.94 | — |
| 15 | 37.57 | 1.09 | — | 38.08 | 1.11 | — | 37.92 | 1.11 | — | 37.90 | 1.04 | — | 37.98 | 0.95 | — |
| 16 | 36.49 | 1.08 | — | 36.91 | 1.17 | — | 36.79 | 1.13 | — | 36.87 | 1.09 | — | 37.03 | 0.95 | — |
| 17 | 35.44 | 1.05 | 24 | 35.71 | 1.20 | 33 | 35.68 | 1.11 | 26 | 35.76 | 1.11 | 27 | 36.05 | 0.98 | 19 |
| 18 | 34.33 | 1.11 | — | 34.53 | 1.18 | — | 34.52 | 1.16 | — | 34.67 | 1.09 | — | 34.82 | 1.23 | — |
| 19 | 33.23 | 1.10 | — | 33.33 | 1.20 | — | 33.37 | 1.13 | — | 33.35 | 1.12 | — | 33.79 | 1.03 | — |
| 20 | 32.13 | 1.10 | — | 32.10 | 1.23 | — | 32.20 | 1.17 | — | 32.41 | 1.14 | — | 32.74 | 1.05 | — |
| 21 | 30.92 | 1.21 | 26 | 30.89 | 1.21 | 32 | 31.00 | 1.20 | 24 | 31.24 | 1.17 | 27 | 31.59 | 1.15 | 21 |
| | Mean | 1.12 | | Mean | 1.15 | | Mean | 1.12 | | Mean | 1.12 | | Mean | 1.07 | |
| | % RSD | 6.8 | | % RSD | 5.1 | | % RSD | 5.2 | | % RSD | 8.6 | | % RSD | 12.9 | |
| | Total (g) | 23.50 | | Total (g) | 24.18 | | Total (g) | 23.41 | | Total (g) | 23.45 | | Total (g) | 22.51 | |
| | | Overall (10) | 1.12 | | | | | | | | | | | | |
| | | % RSD (10) | 8.2 | | | | | | | | | | | | |

“Act.” = actuation. “CW” = can weight in grams. “FH” = foam height in mL. “Dif.” = difference.

TABLE 32
Raw data for Prototype A Placebo - actuation results.
PROTOTYPE A Placebo (Set 6 thru 10)

| | DME - 31 g/2.9 g | | | DME 31 g/3.15 g | | | DME 31 g/3.1 g | | | DME 31 g/3.2 g | | | DME 31 g/2.8 g | | |
|---|---|---|---|---|---|---|---|---|---|---|---|---|---|---|---|
| Act. | | CW | | | CW | | | CW | | | CW | | | CW | |
| # | CW | dif. | FH | CW | dif. | FH | CW | dif. | FH | CW | dif. | FH | CW | dif. | FH |
| 0 | 54.85 | — | — | 54.88 | — | — | 54.57 | — | — | 54.93 | — | — | 54.54 | — | — |
| 1 | 53.38 | 1.47 | 25 | 53.33 | 1.15 | 26 | 53.42 | 1.15 | 31 | 53.71 | 1.22 | 25 | 53.28 | 1.26 | 28 |
| 2 | 52.08 | 1.30 | — | 52.5 | 1.23 | — | 52.16 | 1.26 | — | 52.45 | 1.26 | — | 52.01 | 1.27 | — |
| 3 | 50.80 | 1.28 | — | 51.24 | 1.26 | — | 50.94 | 1.22 | — | 51.20 | 1.25 | — | 50.78 | 1.23 | — |
| 4 | 49.57 | 1.23 | — | 50.03 | 1.21 | — | 49.78 | 1.16 | — | 50.02 | 1.18 | — | 49.56 | 1.22 | — |
| 5 | 48.38 | 1.19 | 32 | 48.95 | 1.08 | 32 | 48.67 | 1.11 | 33 | 48.90 | 1.12 | 35 | 48.47 | 1.09 | 28 |
| 6 | 47.29 | 1.09 | — | 47.88 | 1.07 | — | 47.61 | 1.06 | — | 47.84 | 1.06 | — | 47.38 | 1.09 | — |
| 7 | 46.22 | 1.07 | — | 46.86 | 1.02 | — | 46.56 | 1.05 | — | 46.82 | 1.02 | — | 46.35 | 1.03 | — |
| 8 | 45.21 | 1.01 | — | 45.85 | 1.01 | — | 45.56 | 1.00 | — | 45.77 | 1.05 | — | 45.35 | 1.00 | — |
| 9 | 44.15 | 1.06 | 27 | 44.86 | 0.99 | 28 | 44.51 | 1.05 | 29 | 44.66 | 1.11 | 33 | 44.35 | 1.00 | 24 |
| 10 | 43.12 | 1.03 | — | 43.86 | 1.00 | — | 43.45 | 1.06 | — | 43.59 | 1.07 | — | 43.38 | 0.97 | — |
| 11 | 52.09 | 1.03 | — | 42.87 | 0.99 | — | 42.42 | 1.03 | — | 42.55 | 1.04 | — | 42.36 | 1.02 | — |
| 12 | 41.07 | 1.02 | — | 41.87 | 1.00 | — | 41.37 | 1.05 | — | 41.49 | 1.06 | — | 41.34 | 1.02 | — |
| 13 | 39.87 | 1.20 | 31 | 40.84 | 1.03 | 28 | 40.29 | 1.08 | 28 | 40.38 | 1.11 | 32 | 40.28 | 1.06 | 26 |
| 14 | 38.83 | 1.04 | — | 39.78 | 1.06 | — | 39.20 | 1.09 | — | 39.19 | 1.19 | — | 39.26 | 1.02 | — |
| 15 | 37.71 | 1.12 | — | 38.72 | 1.06 | — | 38.07 | 1.13 | — | 37.99 | 1.20 | — | 38.18 | 1.08 | — |
| 16 | 36.60 | 1.11 | — | 37.66 | 1.06 | — | 36.88 | 1.19 | — | 36.85 | 1.14 | — | 37.13 | 1.05 | — |
| 17 | 35.47 | 1.13 | 31 | 36.53 | 1.13 | 31 | 35.71 | 1.17 | 31 | 35.65 | 1.20 | 33 | 36.00 | 1.13 | 27 |
| 18 | 34.34 | 1.13 | — | 35.38 | 1.15 | — | 34.52 | 1.19 | — | 34.43 | 1.22 | — | 34.83 | 1.17 | — |
| 19 | 33.18 | 1.16 | — | 34.26 | 1.12 | — | 33.29 | 1.23 | — | 33.22 | 1.21 | — | 33.66 | 1.17 | — |
| 20 | 31.96 | 1.22 | — | 33.13 | 1.13 | — | 32.09 | 1.20 | — | 32.01 | 1.31 | — | 32.43 | 1.23 | — |
| 21 | 30.76 | 1.20 | 28 | 31.92 | 1.21 | 31 | 30.84 | 1.25 | 31 | 30.78 | 1.23 | 32 | 31.29 | 1.14 | 26 |
| | Mean | 1.15 | | Mean | 1.09 | | Mean | 1.13 | | Mean | 1.15 | | Mean | 1.11 | |
| | % RSD | 9.9 | | % RSD | 7.7 | | % RSD | 7.0 | | % RSD | 6.7 | | % RSD | 8.6 | |
| | Total (g) | 24.09 | | Total (g) | 22.96 | | Total (g) | 23.73 | | Total (g) | 24.15 | | Total (g) | 23.25 | |

“Act.” = actuation. “CW” = can weight in grams. “FH” = foam height in mL. “Dif.” = difference.

TABLE 33
Raw data for Prototype A Placebo - actuation results.
PROTOTYPE A Placebo (Set 1 thru 5)

| | AP35 - 31 g/2.8 g | | | AP35 - 31 g/3.7 g | | | AP35 - 31 g/3.3 g | | | AP35 - 31 g/3.3 g | | | AP35 - 31 g/3.2 g | | |
|---|---|---|---|---|---|---|---|---|---|---|---|---|---|---|---|
| Act. | | CW | | | CW | | | CW | | | CW | | | CW | |
| # | CW | dif. | FH | CW | dif. | FH | CW | dif. | FH | CW | dif. | FH | CW | dif. | FH |
| 0 | 54.66 | — | — | 55.15 | — | — | 56.97 | — | — | 55.06 | — | — | 55.49 | — | — |
| 1 | 53.47 | 1.19 | 30 | 53.98 | 1.17 | 34 | 55.87 | 1.10 | 39 | 53.76 | 1.30 | 34 | 54.31 | 1.18 | 24 |
| 2 | 52.18 | 1.29 | — | 52.76 | 1.22 | — | 54.67 | 1.20 | — | 52.47 | 1.29 | — | 53.01 | 1.30 | — |
| 3 | 50.92 | 1.26 | — | 51.56 | 1.20 | — | 53.49 | 1.18 | — | 51.09 | 1.38 | — | 51.79 | 1.22 | — |
| 4 | 49.70 | 1.22 | — | 50.39 | 1.17 | — | 52.28 | 1.21 | — | 49.93 | 1.16 | — | 50.61 | 1.18 | — |
| 5 | 48.54 | 1.16 | 26 | 49.28 | 1.11 | 32 | 51.14 | 1.14 | 29 | 48.81 | 1.13 | 37 | 49.54 | 1.07 | 30 |
| 6 | 47.43 | 1.11 | — | 48.20 | 1.08 | — | 50.04 | 1.10 | — | 47.68 | 1.13 | — | 48.48 | 1.06 | — |
| 7 | 46.37 | 1.06 | — | 47.09 | 1.11 | — | 49.01 | 1.03 | — | 46.63 | 1.05 | — | 47.43 | 1.05 | — |
| 8 | 45.28 | 1.09 | — | 45.98 | 1.11 | — | 48.00 | 1.01 | — | 45.60 | 1.03 | — | 46.41 | 1.02 | — |
| 9 | 44.24 | 1.04 | 22 | 44.87 | 1.11 | 31 | 46.88 | 1.12 | 29 | 44.50 | 1.10 | 29 | 45.27 | 1.14 | 30 |
| 10 | 43.19 | 1.05 | — | 43.72 | 1.15 | — | 45.71 | 1.17 | — | 43.39 | 1.11 | — | 44.22 | 1.05 | — |
| 11 | 42.14 | 1.05 | — | 42.53 | 1.19 | — | 44.63 | 1.08 | — | 42.28 | 1.11 | — | 43.16 | 1.06 | — |
| 12 | 41.10 | 1.04 | — | 41.39 | 1.14 | — | 43.48 | 1.15 | — | 41.11 | 1.17 | — | 42.09 | 1.07 | — |
| 13 | 40.04 | 1.06 | 22 | 40.24 | 1.15 | 32 | 42.28 | 1.20 | 29 | 39.90 | 1.21 | 31 | 40.95 | 1.14 | 30 |
| 14 | 38.98 | 1.06 | — | 39.04 | 1.20 | — | 41.09 | 1.19 | — | 38.62 | 1.28 | — | 39.82 | 1.13 | — |
| 13 | 37.89 | 1.09 | — | 37.82 | 1.22 | — | 39.90 | 1.19 | — | 37.38 | 1.24 | — | 38.62 | 1.20 | — |
| 16 | 36.74 | 1.15 | — | 36.55 | 1.27 | — | 38.63 | 1.25 | — | 36.10 | 1.28 | — | 37.42 | 1.20 | — |
| 17 | 35.62 | 1.12 | 23 | 35.31 | 1.24 | 33 | 37.34 | 1.31 | 31 | 34.82 | 1.28 | 35 | 36.20 | 1.22 | 32 |
| 18 | 34.50 | 1.12 | — | 34.08 | 1.23 | — | 36.08 | 1.26 | — | 33.55 | 1.27 | — | 34.98 | 1.22 | — |
| 19 | 33.33 | 1.17 | — | 32.82 | 1.26 | — | 34.79 | 1.29 | — | 32.27 | 1.28 | — | 33.74 | 1.24 | — |
| 20 | 32.15 | 1.18 | — | 31.54 | 1.28 | — | 33.49 | 1.30 | — | 31.01 | 1.26 | — | 32.51 | 1.23 | — |
| 21 | 30.98 | 1.17 | 26 | 30.27 | 1.27 | 34 | 32.32 | 1.17 | 30 | 29.74 | 1.27 | 32 | 31.24 | 1.27 | 31 |
| | Mean | 1.13 | | Mean | 1.18 | | Mean | 1.17 | | Mean | 1.21 | | Mean | 1.15 | |
| | % RSD | 6.5 | | % RSD | 5.2 | | % RSD | 7.0 | | % RSD | 7.9 | | % RSD | 7.2 | |
| | Total (g) | 23.68 | | Total (g) | 24.88 | | Total (g) | 24.65 | | Total (g) | 25.32 | | Total (g) | 24.25 | |
| | | Overall (10) | 1.16 | | | | | | | | | | | | |
| | | % RSD (10) | 7.7 | | | | | | | | | | | | |

“Act.” = actuation. “CW” = can weight in grams. “FH” = foam height in mL. “Dif.” = difference.

TABLE 34
Raw data for Prototype A Placebo - actuation results.
PROTOTYPE A Placebo (Set 6 thru 10)

| | AP35 - 31 g/3.0 g | | | AP35 - 31 g/3.3 g | | | AP35 - 31 g/3.2 g | | | AP35 - 31 g/3.1 g | | | AP35 - 31 g/2.4 g | | |
|---|---|---|---|---|---|---|---|---|---|---|---|---|---|---|---|
| | | CW | | | CW | | | CW | | | CW | | | CW | |
| | CW | dif. | FH | CW | dif. | FH | CW | dif. | FH | CW | dif. | FH | CW | dif. | FH |
| 0 | 54.48 | — | — | 55.05 | — | — | 54.98 | — | — | 54.61 | — | — | 54.19 | — | — |
| 1 | 53.28 | 1.20 | 32 | 53.94 | 1.11 | 25 | 53.38 | 1.20 | 30 | 53.50 | 1.11 | 24 | 53.00 | 1.19 | 26 |
| 2 | 52.01 | 1.27 | — | 52.62 | 1.32 | — | 52.32 | 1.26 | — | 52.20 | 1.30 | — | 51.78 | 1.22 | — |
| 3 | 50.75 | 1.26 | — | 51.36 | 1.26 | — | 51.30 | 1.22 | — | 50.97 | 1.23 | — | 50.58 | 1.20 | — |
| 4 | 49.38 | 1.17 | — | 50.15 | 1.21 | — | 50.14 | 1.16 | — | 49.80 | 1.17 | — | 49.45 | 1.13 | — |
| 5 | 48.49 | 1.09 | 28 | 49.10 | 1.05 | 31 | 48.95 | 1.19 | 33 | 48.84 | 1.16 | 32 | 48.33 | 1.12 | 21 |
| 6 | 47.44 | 1.05 | — | 47.95 | 1.14 | — | 47.89 | 1.06 | — | 47.59 | 1.05 | — | 47.33 | 1.00 | — |
| 7 | 46.42 | 1.02 | — | 46.91 | 1.05 | — | 46.87 | 1.02 | — | 46.59 | 1.00 | — | 46.33 | 1.00 | — |
| 8 | 45.28 | 1.00 | — | 45.92 | 0.99 | — | 45.88 | 0.99 | — | 45.43 | 1.16 | — | 45.27 | 1.06 | — |
| 9 | 44.36 | 1.06 | 25 | 44.73 | 1.19 | 33 | 44.68 | 1.20 | 32 | 44.22 | 1.21 | 32 | 44.32 | 0.95 | 19 |
| 10 | 43.34 | 1.02 | — | 43.67 | 1.06 | — | 43.67 | 1.01 | — | 43.18 | 1.04 | — | 43.34 | 0.98 | — |
| 11 | 42.28 | 1.06 | — | 42.60 | 1.07 | — | 42.63 | 1.04 | — | 42.14 | 1.04 | — | 42.23 | 1.11 | — |
| 12 | 41.22 | 1.06 | — | 41.51 | 1.09 | — | 41.58 | 1.05 | — | 41.09 | 1.05 | — | 41.08 | 1.15 | — |
| 13 | 40.11 | 1.11 | 27 | 40.26 | 1.25 | 34 | 40.32 | 1.26 | 32 | 39.86 | 1.23 | 31 | 40.04 | 1.04 | 20 |
| 14 | 38.96 | 1.15 | — | 39.11 | 1.15 | — | 39.21 | 1.11 | — | 38.74 | 1.12 | — | 38.85 | 1.19 | — |
| 15 | 37.80 | 1.16 | — | 37.92 | 1.19 | — | 38.06 | 1.15 | — | 37.55 | 1.19 | — | 37.67 | 1.18 | — |
| 16 | 36.59 | 1.21 | — | 36.66 | 1.26 | — | 36.86 | 1.20 | — | 36.37 | 1.18 | — | 36.47 | 1.20 | — |
| 17 | 35.41 | 1.18 | 29 | 35.38 | 1.28 | 34 | 35.60 | 1.26 | 33 | 35.14 | 1.23 | 32 | 35.55 | 0.92 | 16 |
| 18 | 34.21 | 1.20 | — | 34.14 | 1.24 | — | 34.39 | 1.21 | — | 33.95 | 1.19 | — | 34.58 | 0.97 | — |
| 19 | 33.02 | 1.19 | — | 32.39 | 1.25 | — | 33.15 | 1.24 | — | 32.73 | 1.22 | — | 33.57 | 1.01 | — |
| 20 | 31.80 | 1.22 | — | 31.66 | 1.23 | — | 31.91 | 1.24 | — | 31.54 | 1.19 | — | 32.47 | 1.10 | — |
| 21 | 30.61 | 1.19 | 27 | 30.37 | 1.29 | 32 | 30.66 | 1.25 | 30 | 30.27 | 1.27 | 30 | 31.25 | 1.22 | 21 |
| | Mean | 1.14 | | Mean | 1.18 | | Mean | 1.16 | | Mean | 1.16 | | Mean | 1.09 | |
| | % RSD | 7.3 | | % RSD | 8.1 | | % RSD | 8.0 | | % RSD | 7.2 | | % RSD | 9.0 | |
| | Total (g) | 23.87 | | Total (g) | 24.68 | | Total (g) | 24.32 | | Total (g) | 24.34 | | Total (g) | 22.74 | |

“Act.” = actuation. “CW” = can weight in grams. “FH” = foam height in mL. “Dif.” = difference.

TABLE 35
Raw data for Prototype A Placebo - actuation results.
PROTOTYPE A (Set 1 thru 5)

| | AP70 Placebo Can 1 | | AP Placebo Can 2 | | AP Placebo Can 3 | | AP Placebo Can 4 | | AP70 Placebo 1 M 25 C. | |
|---|---|---|---|---|---|---|---|---|---|---|
| Act. | | CW | | CW | | CW | | CW | | CW |
| # | CW | Dif. | CW | Dif. | CW | Dif. | CW | Dif. | CW | Dif. |
| 0 | 54.22 | — | 54.42 | — | 55.64 | — | 54.19 | — | 54.06 | — |
| 1 | 53.13 | 1.09 | 53.42 | 1.00 | 54.25 | 1.39 | 53.1 | 1.09 | 52.94 | 1.12 |
| 2 | 51.90 | 1.23 | 52.34 | 1.08 | 53.09 | 1.16 | 51.94 | 1.16 | 51.76 | 1.18 |
| 3 | 50.74 | 1.16 | 51.31 | 1.03 | 52.01 | 1.08 | 50.74 | 1.20 | 50.56 | 1.20 |
| 4 | 49.64 | 1.10 | 50.31 | 1.00 | 50.92 | 1.09 | 49.50 | 1.24 | 49.42 | 1.14 |
| 5 | 48.61 | 1.03 | 49.35 | 0.96 | 49.86 | 1.06 | 48.35 | 1.15 | 48.34 | 1.08 |
| 6 | 47.58 | 1.03 | 48.41 | 0.94 | 48.80 | 1.06 | 47.19 | 1.16 | 47.28 | 1.06 |
| 7 | 46.46 | 1.12 | 47.48 | 0.93 | 47.74 | 1.06 | 46.09 | 1.10 | 46.18 | 1.10 |
| 8 | 45.31 | 1.15 | 46.54 | 0.94 | 46.68 | 1.06 | 45.03 | 1.06 | 44.98 | 1.20 |
| 9 | 44.20 | 1.11 | 45.64 | 0.90 | 45.71 | 0.97 | 44.05 | 0.98 | 43.92 | 1.06 |
| 10 | 43.07 | 1.13 | 44.44 | 1.20 | 44.59 | 1.12 | 43.09 | 0.96 | 42.70 | 1.22 |
| 11 | 41.97 | 1.10 | 43.35 | 1.09 | 43.57 | 1.02 | 42.15 | 0.94 | 41.74 | 0.96 |
| 12 | 40.83 | 1.14 | 42.24 | 1.11 | 42.50 | 1.07 | 41.22 | 0.93 | 40.74 | 1.00 |
| 13 | 39.67 | 1.16 | 41.12 | 1.12 | 41.44 | 1.06 | 40.32 | 0.90 | 39.68 | 1.06 |
| 14 | 38.49 | 1.18 | 39.97 | 1.15 | 40.39 | 1.05 | 39.41 | 0.91 | 38.50 | 1.18 |
| 15 | 32.32 | 1.17 | 38.80 | 1.17 | 39.30 | 1.09 | 38.55 | 0.86 | 37.62 | 0.88 |
| 16 | 36.13 | 1.19 | 37.64 | 1.16 | 38.24 | 1.06 | 37.71 | 0.84 | 36.40 | 1.22 |
| 17 | 34.94 | 1.19 | 36.49 | 1.15 | 37.19 | 1.05 | 36.90 | 0.81 | 35.20 | 1.20 |
| 18 | 33.78 | 1.16 | 35.31 | 1.18 | 36.11 | 1.08 | 36.13 | 0.77 | 34.20 | 1.00 |
| 19 | 32.60 | 1.18 | 34.13 | 1.18 | 34.98 | 1.13 | 35.44 | 0.69 | 33.06 | 1.14 |
| 20 | 31.44 | 1.16 | 32.91 | 1.22 | 33.90 | 1.08 | 34.75 | 0.69 | 31.86 | 1.20 |
| 21 | 30.26 | 1.18 | 31.72 | 1.19 | 32.70 | 1.20 | 34.08 | 0.67 | 30.76 | 1.10 |
| | Mean | 1.14 | Mean | 1.08 | Mean | 1.09 | Mean | 0.96 | Mean | 1.11 |
| | % RSD | 4.5 | % RSD | 9.6 | % RSD | 7.6 | % RSD | 18.4 | % RSD | 8.5 |
| | Total (g) | 23.96 | Total (g) | 22.7 | Total (g) | 22.94 | Total (g) | 20.11 | Total (g) | 23.3 |
| Overall (10) | 1.12 | | | | | | | | | |
| % RSD (10) | 12.9 | | | | | | | | | |

“Act.” = actuation. “CW” = can weight in grams. “FH” = foam height in mL. “Dif.” = difference.

TABLE 36
Raw data for Prototype A Placebo and Active - actuation results.
PROTOTYPE A (Set 6 thru 10)

| | AP70 30 μg 1 M 25 C. | | AP70 3000 μg 1 M 25 C. | | AP70 Placebo 1 M 5 C. | | AP70 30 μg 1 M 5 C. | | AP70 3000 μg 1 M 5 C. | |
|---|---|---|---|---|---|---|---|---|---|---|
| Act. | | CW | | CW | | CW | | CW | | CW |
| # | CW | Dif. | CW | Dif. | CW | Dif. | CW | Dif. | CW | Dif. |
| 0 | 53.40 | — | 53.28 | — | 54.54 | — | 53.64 | — | 53.44 | — |
| 1 | 52.44 | 0.96 | 52.24 | 1.04 | 53.58 | 0.96 | 52.44 | 1.20 | 52.14 | 1.30 |
| 2 | 51.34 | 1.10 | 50.96 | 1.28 | 52.40 | 1.18 | 51.16 | 1.28 | 51.66 | 0.48 |
| 3 | 50.18 | 1.16 | 49.68 | 1.28 | 51.18 | 1.22 | 49.86 | 1.30 | 50.38 | 1.28 |
| 4 | 49.10 | 1.08 | 48.44 | 1.24 | 49.98 | 1.20 | 48.56 | 1.30 | 48.70 | 1.68 |
| 5 | 48.08 | 1.02 | 47.24 | 1.20 | 48.78 | 1.20 | 47.32 | 1.24 | 47.40 | 1.30 |
| 6 | 47.02 | 1.06 | 46.12 | 1.12 | 47.72 | 1.06 | 46.08 | 1.24 | 46.32 | 1.08 |
| 7 | 45.78 | 1.24 | 44.88 | 1.24 | 46.56 | 1.16 | 44.82 | 1.26 | 45.08 | 1.24 |
| 8 | 44.60 | 1.18 | 43.60 | 1.28 | 45.34 | 1.22 | 43.58 | 1.24 | 43.96 | 1.32 |
| 9 | 43.34 | 1.26 | 42.34 | 1.26 | 44.28 | 1.06 | 42.36 | 1.22 | 43.02 | 0.94 |
| 10 | 42.24 | 1.10 | 41.24 | 1.10 | 43.08 | 1.20 | 41.18 | 1.18 | 41.78 | 1.24 |
| 11 | 41.18 | 1.06 | 40.14 | 1.10 | 42.00 | 1.08 | 39.94 | 1.24 | 40.58 | 1.20 |
| 12 | 39.94 | 1.24 | 39.08 | 1.06 | 40.76 | 1.24 | 38.72 | 1.22 | 39.82 | 0.76 |
| 13 | 39.06 | 0.88 | 37.82 | 1.26 | 39.72 | 1.04 | 37.36 | 1.16 | 39.10 | 0.72 |
| 14 | 38.04 | 1.02 | 36.78 | 1.04 | 38.50 | 1.22 | 36.36 | 1.20 | 37.64 | 1.46 |
| 15 | 37.06 | 0.98 | 35.90 | 0.88 | 37.34 | 1.16 | 35.14 | 1.22 | 36.56 | 1.08 |
| 16 | 35.82 | 1.24 | 34.62 | 1.28 | 36.12 | 1.22 | 33.92 | 1.22 | 35.00 | 1.56 |
| 17 | 34.60 | 1.22 | 33.42 | 1.20 | 34.90 | 1.22 | 32.66 | 1.26 | 33.8 | 1.20 |
| 18 | 33.42 | 1.18 | 32.48 | 0.94 | 33.78 | 1.12 | 31.40 | 1.26 | 32.68 | 1.12 |
| 19 | 32.24 | 1.18 | 31.32 | 1.16 | 32.56 | 1.22 | 30.12 | 1.28 | 31.42 | 1.26 |
| 20 | 31.00 | 1.24 | 30.18 | 1.14 | 31.34 | 1.22 | 28.94 | 1.18 | 30.16 | 1.26 |
| 21 | 29.80 | 1.20 | 29.06 | 1.12 | 30.14 | 1.20 | | | 28.96 | 1.20 |
| | Mean | 1.12 | Mean | 1.15 | Mean | 1.16 | Mean | 1.24 | Mean | 1.17 |
| | % RSD | 9.7 | % RSD | 10.1 | % RSD | 6.7 | % RSD | 3.2 | % RSD | 23.4 |
| | Total (g) | 23.60 | Total (g) | 24.22 | Total (g) | 24.4 | Total (g) | 24.7 | Total (g) | 24.48 |

“Act.” = actuation. “CW” = can weight in grams. “FH” = foam height in mL. “Dif.” = difference.

### Example 19. Microbial Examination

The table below shows the results of the microbiological examination. Based on this data, the enumeration portion of this method is suitable for these test organisms. This method is suitable for enumerating microorganisms which may be contaminating the product.

TABLE 37
Microbiological examination of nonsterile products: microbial
enumeration tests for Prototype A placebo. Lots were tested in triplicate.
Microbiological Examination Of Nonsterile Products: Microbial Enumeration Tests

| | | Total Aerobic Microbial | Total Yeasts and Molds |
|---|---|---|---|
| | | Count | Count |
| Method | Organisms | (MCTA) | (SDA) |
| 74.8000 | E. coli ATCC 8739 | 1:10 | |
| | P. aeruginosa ATCC 9027 | 1:10 | |
| | S. aureus ATCC 6538 | 1:10 | |
| | B. subtilis ATCC 6633 | 1:10 | |
| | C. albicans ATCC 10231 | 1:10 | 1:10 |
| | A. brasiliensis ATCC | 1:10 | 1:10 |
| | 16404 | | |

Validated Standard Methods of Analysis: 74.8001
Suitability method is equivalent to USP Microbial Enumeration Tests in USP general chapter <61>

TABLE 38
Microbiological examination of nonsterile products: microbial
enumeration tests for Prototype A pharmaceutical rectal foam
composition, 100 μg. Lots were tested in triplicate.
Microbiological Examination Of Nonsterile Products: Microbial Enumeration Tests

| | | Total Aerobic Microbial | Total Yeasts and Molds |
|---|---|---|---|
| | | Count | Count |
| Method | Organisms | (MCTA) | (SDA) |
| 74.8000 | E. coli ATCC 8739 | 1:10 | |
| | P. aeruginosa ATCC 9027 | 1:10 | |
| | S. aureus ATCC 6538 | 1:10 | |
| | B. subtilis ATCC 6633 | 1:10 | |
| | C. albicans ATCC 10231 | 1:10 | 1:10 |
| | A. brasiliensis ATCC | 1:10 | 1:10 |
| | 16404 | | |

Validated Standard Methods of Analysis: 74.8001
Suitability method is equivalent to USP Microbial Enumeration Tests in USP general chapter <61>

Based on this data, the enumeration portion of this method is suitable for these test organisms. This method is suitable for enumerating microorganisms which may be contaminating the product

TABLE 39
Microbiological examination of nonsterile products: microbial
enumeration tests for Prototype A pharmaceutical rectal foam
composition, 300 μg. Lots were tested in triplicate.
Microbiological Examination Of Nonsterile Products: Microbial Enumeration Tests

| | | Total Aerobic Microbial | Total Yeasts and Molds |
|---|---|---|---|
| | | Count | Count |
| Method | Organisms | (MCTA) | (SDA) |
| 74.8000 | E. coli ATCC 8739 | 1:10 | |
| | P. aeruginosa ATCC 9027 | 1:10 | |
| | S. aureus ATCC 6538 | 1:10 | |
| | B. subtilis ATCC 6633 | 1:10 | |
| | C. albicans ATCC 10231 | 1:10 | 1:10 |
| | A. brasiliensis ATCC | 1:10 | 1:10 |
| | 16404 | | |

Validated Standard Methods of Analysis: 74.8001
Suitability method is equivalent to USP Microbial Enumeration Tests in USP general chapter <61>

Based on this data, the enumeration portion of this method is suitable for these test organisms. This method is suitable for enumerating microorganisms which may be contaminating the product.

### Example 20. Fifty-Month Stability Assay

A 50-month stability assay was performed. Stability of the pharmaceutical rectal foam composition comprising the peptide of the present disclosure was evaluated at various temperatures. Samples were stored in Wheaton W224682 4 mL Lab File® amber glass bottles with PTFE screw cap seal liners. Proposed long term storage is −20±5° C. Data was also obtained for accelerated conditions at 5° C.±3° C. and stress conditions at 25° C.±2° C. with 60±5% relative humidity. The release testing sample (LIMS #1512-0061) serves as the initial data point for all testing. In the tables below, “RRT”=relative retention time.

TABLE 40
Stability tests, conditions and time points. The stability matrix and storage temperature,
tests to be run, specifications to be met, and testing time points, are shown here.

| | | | | Spec/ | | | | | | | | | | | | |
|---|---|---|---|---|---|---|---|---|---|---|---|---|---|---|---|---|
| Analytical | Analytical | Units/ | Sample | Comp | Storage | | Time point (months) | | | | | | | | | |
| Method | Property | Qualifier | Size | Value | Conditions | | ½ | 1 | 3 | 6 | 9 | 12 | 18 | 24 | 36 | 48 |
| A/VI | Appearance | N/A | 1 bottle | White to | −20° | C. | — | — | X | X | X | X | X | X | X | x |
| | | | | off-white | 2-8° | C. | — | X | X | X | — | — | — | — | — | — |
| | | | | Powder | 25° C./60% RH | | X | X | — | — | — | — | — | — | — | — |
| T1830 | Purity | Percent | | NLT 95% | −20° | C. | — | — | X | X | X | X | X | X | X | X |
| (RP-HPLC) | RRT 1.05 Impurity: | Percent | | NMT 2% | 2-8° | C. | — | X | X | X | — | — | — | — | — | — |
| | Total | Percent | | NMT 5% | 25° C./60% RH | | X | X | — | — | — | — | — | — | — | — |
| | Related | | | | | | | | | | | | | | | |
| | Substances/Impurities | | | | | | | | | | | | | | | |
| | Largest Unspecified | Percent | | NMT 1% | | | | | | | | | | | | |
| **T1905 | Purity | Percent | # | Report | −20° | C. | — | — | X | X | X | X | X | X | X | X |
| (SEC) | | Percent | | Result | 2-8° | C. | — | X | X | X | — | — | — | — | — | — |
| | Total | | | Report | 25° C./60% RH | | X | X | — | — | — | — | — | — | — | — |
| | Multimers | | | Result | | | | | | | | | | | | |
| G142 | Water | Percent | | Report | −20° | C. | — | — | X | X | X | X | X | X | X | X |
| (KF) | | | | Result | 2-8° | C. | — | X | X | X | — | — | — | — | — | — |
| | | | | | 25° C./60% RH | | X | X | — | — | — | — | — | — | — | — |
| MLM200 = | Bioburden | CFU/0.1 | 1 bottle | *TAMC NMT | −20° | C. | — | — | — | — | — | X | — | — | — | — |
| 20 | | gram | | 2000 *TYMC | 2-8° | C. | — | — | — | — | — | — | — | — | — | — |
| (Bioburden) | | | | NMT 200 | 25° C./60% RH | | — | — | — | — | — | — | — | — | — | — |
| DSC | Differential Scanning | N/A | # | Report | −20° | C. | — | — | — | X | — | X | X | X | X | X |
| | Calorimetry | | | Data | 2-8° | C. | — | — | — | X | — | — | — | — | — | — |

| | | | | | | | | | | |
|---|---|---|---|---|---|---|---|---|---|---|
| 25° C./60% RH | — | X | — | — | — | — | — | — | — | — |

# = Vials shared across multiple tests.
NLT = Not Less Than,
NMT = Not More Than,
— = testing not scheduled.
*= Total Aerobic Microbial Count, Total Yeast and Mold Count respectively; Units in CFU/g.
**= SEC Method.
ND = not detected.
NT = Not Tested.
“—” Test not performed.

TABLE 41
Stability data results at proposed long term storage conditions (−20° C.) for peptide drug substance - Part
I. Container/closure description: Wheaton W224682, 4 mL Lab File amber glass bottles with PTFE screw cap seal liners.

| | | | Initial | 3 M | 6 M | 9 M | 12 M |
|---|---|---|---|---|---|---|---|
| | | | Pull Date | | | | |
| | | Acceptance | Feb. 8, 2016 | May 10, 2016 | Aug. 14, 2016 | Nov. 8, 2016 | Feb. 7, 2017 |
| Test | Method | Criteria | RESULTS | | | | |
| Description | Visual | White to off- | Conforms | Conforms | Conforms | Conforms | Conforms |
| | inspection | white oval | | | | | |
| | | shaped tablets | | | | | |
| Identity | HPLC | Retention time | Conforms | Conforms | Conforms | Conforms | Conforms |
| | | consistent with | | | | | |
| | | reference standard | | | | | |
| | | ≥95% | 97.6 | 98.2 | 98.2 | 98.4 | 98.3 |
| Purity (area %) | | Total Impurities <5% | 2.4 | 1.8 | 1.8 | 1.6 | 1.7 |
| Related | | | | | | | |

| | | | | | | |
|---|---|---|---|---|---|---|
| Substances | RRT 1.05 ≤2% | 0.93 | 0.82 | 0.80 | 0.81 | 0.82 |

| | RRT | % Area | RRT | % Area | RRT | % Area | RRT | % Area | RRT | % Area |
|---|---|---|---|---|---|---|---|---|---|---|
| Other | 0.80 | 0.07 | — | — | 0.82 | 0.05 | 0.82 | 0.05 | — | — |
| Individual | 0.84 | 0.14 | 0.83 | 0.10 | 0.83 | 0.12 | 0.83 | 0.11 | 0.83 | 0.12 |
| Impurities ≤1% | 0.86 | 0.13 | 0.86 | 0.18 | 0.86 | 0.06 | — | — | — | — |
| | 0.89 | 0.35 | 0.88 | 0.39 | 0.88 | 0.32 | 0.88 | 0.31 | 0.88 | 0.31 |
| | 1.02 | 0.40 | 1.02 | 0.09 | 1.02 | 0.22 | 1.02 | 0.08 | 1.02 | 0.23 |
| | 1.06 | 0.26 | 1.06 | 0.20 | 1.06 | 0.18 | 1.06 | 0.18 | 1.06 | 0.16 |
| | 1.07 | 0.09 | 1.07 | 0.06 | 1.07 | 0.06 | 1.07 | 0.06 | 1.07 | 0.06 |
| | — | — | — | — | — | — | — | — | — | — |
| | — | — | — | — | — | — | — | — | — | — |

| Water Content | Karl Fischer | Report Result (%) | 2.56 | 4.40 | 7.42 | 3.84 | 3.04 |
|---|---|---|---|---|---|---|---|
| Multimer Content | SEC | Report Result (Area %) | 0.05 | ND | ND | 0.14 | 0.09 |
| Differential | DSC | Report Results | Consistent with | NT | Consistent with | NT | Consistent with |
| Scanning | | | amorphous | | amorphous | | amorphous |
| Calorimetry | | | | | | | |
| Microbial | USP <61> | Molds/ | <4 | NT | NT | NT | <4 |
| Limits | | Yeast <10² cfu/g | | | | | |

| Total | 4 | NT | NT | NT | 4 |
|---|---|---|---|---|---|
| Aerobic <10³ cfu/g | | | | | |

TABLE 42
Stability data results at proposed long term storage conditions (−20° C.) for peptide drug substance - Part
II. Container/closure description: Wheaton W224682, 4 mL Lab File amber glass bottles with PTFE screw cap seal liners.

| | | | 18 M | | 24 M | | 36 M | | 50 M | | 60 M | |
|---|---|---|---|---|---|---|---|---|---|---|---|---|
| | | | Pall Date | | | | | | | | | |
| | | Acceptance | Aug. 14, 2017 | | Feb. 12, 2018 | | Feb. 11, 2019 | | Apr. 15, 2020 | | — | |
| Test | Method | Criteria | RESULTS | | | | | | | | | |
| Description | Visual | shaped tablets | Conforms | | White to off-white | | Conforms | | Conforms | | — | |
| | inspection | | | | oval Conforms | | | | | | | |
| Identity | HPLC | Retention time | Conforms | | Conforms | | Conforms | | Conforms | | — | |
| | | consistent with | | | | | | | | | | |
| | | reference standard | | | | | | | | | | |
| Purity (area %) | | ≥95% | 97.7 | | 97.6 | | 98.0 | | 98.8 | | — | |
| Related | | Total Impurities ≤5% | 2.26 | | 2.38 | | 2.0 | | 1.3 | | — | |
| Substances | | RRT 1.05 ≤2% | 0.91 | | 0.94 | | 0.91 | | 0.73 | | — | |
| | | | RRT | % Area | RRT | % Area | RRT | % Area | RRT | % Area | RRT | % Area |
| | | Other | 0.81 | 0.06 | 0.83 | 0.14 | 0.82 | 0.05 | 0.83 | 0.12 | — | — |
| | | Individual | 0.83 | 0.13 | 0.85 | 0.06 | 0.84 | 0.12 | — | — | — | — |
| | | Impurities ≤1% | 0.85 | 0.05 | 0.86 | 0.07 | 0.88 | 0.35 | 0.88 | 0.32 | — | — |
| | | | 0.86 | 0.08 | 0.88 | 0.34 | 1.02 | 0.15 | — | — | — | — |
| | | | 0.88 | 0.35 | 1.02 | 0.27 | 1.03 | 0.05 | — | — | — | — |
| | | | 1.02 | 0.27 | 1.03 | 0.33 | — | — | 1.03 | 0.09 | — | — |
| | | | 1.03 | 0.08 | 1.056 | 0.16 | — | — | — | — | — | — |
| | | | 1.06 | 0.24 | 1.062 | 0.15 | 1.06 | 0.22 | — | — | — | — |
| | | | 1.07 | 0.10 | 1.07 | 0.13 | 1.07 | 0.10 | — | — | — | — |
| Water Content | Karl Fischer | Report Result (%) | 3.42 | | 3.83 | | 2.85 | | 3.60 | | — | |
| Multimer | SEC | Report Result | 0.22 | | 0.21 | | 0.16 | | 0.13 | | — | |
| Content | | (Area 3%) | | | | | | | | | | |
| Differential | DSC | Report Results | Consistent with | | Consistent with | | Consistent with | | Consistent with | | — | |
| Scanning | USP <921> | | Amorphous | | Amorphous | | Amorphous | | Amorphous | | | |
| Calorimetry | | | | | | | | | | | | |
| Microbial | USP <61> | Molds | NT | | NT | | NT | | NT | | — | |
| Limits | | Yeast <10² cfu/g | | | | | | | | | | |

| | | | | | |
|---|---|---|---|---|---|
| Total | NT | NT | NT | NT | — |
| Aerobic <10² cfu/g | | | | | |

TABLE 43
Stability data results at accelerated storage conditions (2-8° C.) for peptide drug. Container/closure
description: Wheaton W224682, 4 mL Lab File amber glass bottles with PTFE screw cap seal liners.

| | | | Initial | | 1 M | | 3 M | | 6 M | |
|---|---|---|---|---|---|---|---|---|---|---|
| | | | Pull Date | | | | | | | |
| | | Acceptance | Feb. 8, 2016 | | Mar. 9, 2016 | | May 10, 2016 | | Aug. 14, 2016 | |
| Test | Method | Criteria | RESULTS | | | | | | | |
| Description | Visual | White to off-white oval | Conforms | | Conforms | | Conforms | | Conforms | |
| | inspection | shaped tablets | | | | | | | | |
| Identity | HPLC | Retention time | Conforms | | Conforms | | Conforms | | Conforms | |
| | | consistent with | | | | | | | | |
| | | reference standard | | | | | | | | |
| Purity (ares %) | | ≥95% | 97.6 | | 98.5 | | 98.3 | | 98.3 | |
| Related | | Total Impurities ≤5% | 2.4 | | 1.5 | | 1.7 | | 1.7 | |
| Substances | | KKT 1.05 ≤2% | 0.93 | | 0.79 | | 0.83 | | 0.82 | |
| | | | RRT | % Area | RRT | % Area | RRT | % Area | RRt | % Area |
| | | Other | 0.80 | 0.07 | 0.83 | 0.15 | 0.80 | 0.07 | — | — |
| | | Individual | 0.84 | 0.14 | — | — | 0.84 | 0.14 | 0.83 | 0.12 |
| | | Impurities ≤1% | 0.86 | 0.13 | 0.88 | 0.31 | 0.86 | 0.13 | — | — |
| | | | 0.89 | 0.35 | — | — | 0.89 | 0.35 | 0.88 | 0.31 |
| | | | 1.02 | 0.40 | 1.02 | 0.14 | 1.02 | 0.40 | 1.02 | 0.21 |
| | | | 1.06 | 0.26 | 1.06 | 0.07 | 1.06 | 0.26 | 1.06 | 0.17 |
| | | | 1.07 | 0.09 | — | — | 1.07 | 0.09 | 1.07 | 0.06 |
| | | | | | — | — | — | — | — | — |
| Water Content | Karl Fischer | Report Result (X) | 2.56 | | 3.41 | | 4.67 | | 9.10 | |
| Multinter | SEC | Report Result (Area %) | 0.05 | | 50.05 | | 50.05 | | ND | |
| Content | | | | | | | | | | |
| Differential | DSC | Report Results | Consistent with | | NT | | NT | | Consistent with | |
| Scanning | USP <921> | | amorphous | | | | | | amorphous | |
| Calorimetry | | | | | | | | | | |

| | | | | | | |
|---|---|---|---|---|---|---|
| Microbial Limits | USP <613 | Molds | <4 | NT | NT | NT |

| Yeast <10² cfu/g | | | |
|---|---|---|---|
| Total | 4 | NT | NT |
| Aerobic <10² cfu/g | | | |

TABLE 44
Stability data results at accelerated storage conditions (25° C./60% RH) for peptide drug substance. Container/closure
description: Wheaton W224682, 4 mL Lab File amber glass bottles with PTFE screw cap seal liners.

| | | | Initial | | 2 weeks | | 4 weeks | |
|---|---|---|---|---|---|---|---|---|
| | | | Pull Date | | | | | |
| | | | Feb. 8, 2016 | | Feb. 25, 2016 | | Mar. 9, 2016 | |
| Test | Method | Acceptance Criteria | RESULTS | | | | | |
| Description | Visual | White to off-white oval shaped | Conforms | | Conforms | | Conforms | |
| | inspection | tablets | | | | | | |
| Identity | HPLC | Retention time consistent with | Conforms | | Conforms | | Conforms | |
| | | reference standard | | | | | | |
| Purity (area %) | | ≥95% | 97.6 | | 98.7 | | 98.5 | |
| Related | | Total Impurities ≤5% | 2.4 | | 1.3 | | 1.5 | |
| Substances | | RRT 1.05 ≤2% | 0.93 | | 0.79 | | 0.76 | |
| | | | RRT | % Area | RRT | % Area | RRT | % Area |
| | | Other | 0.80 | 0.07 | — | ND | 0.66 | 0.06 |
| | | Individual | 0.84 | 0.14 | 0.83 | 0.13 | 0.71 | 0.06 |
| | | Impurities ≤1% | 0.86 | 0.13 | ND | ND | 0.82 | 0.09 |
| | | | 0.89 | 0.35 | 0.88 | 0.25 | 0.83 | 0.11 |
| | | | 1.02 | 0.40 | 1.02 | 0.08 | 0.88 | 0.26 |
| | | | 1.06 | 0.26 | 1.06 | 0.05 | 0.97 | 0.06 |
| | | | 1.07 | 0.09 | ND | ND | 1.02 | 0.08 |
| | | | — | — | — | — | — | — |
| | | | — | — | — | — | — | — |
| Water Content | Karl Fischer | Report Result (%) | 2.56 | | 3.27 | | 3.38 | |
| Multimer | SEC | Report Result (Area %) | 0.05 | | ≤0.05 | | ≤0.05 | |
| Content | | | | | | | | |
| Differential | DSC | Report Results | Consistent with | | NT | | Consistent with | |
| Scanning | USP <921> | | amorphous | | | | amorphous | |
| Calorimetry | | | | | | | | |

| | | | | | |
|---|---|---|---|---|---|
| Microbial | USP <61> | Molds/Yeast <10² cfu/g | <4 | NT | NT |
| Limits | | Total Aerobic <10² cfu/g | 4 | NT | NT |

### Example 21. Overview of Development of Pharmaceutical Rectal Foam

A pharmaceutical rectal foam composition of the present disclosure was created comprising an oil-in-water emulsion having the following components: a peptide of the present disclosure, i.e., a peptide having an amino acid sequence of Cys₁-Cth₂-Glu₃-Leu₄-Cys₅-Cys₆-Asn₇-Val₈-Ala₉-Cys₁₀-Tyr₁₁-Gly₁₂-Cys₁₃ (SEQ ID NO: 1), wherein Cth is a cystathionine; wherein Cys₁ and Cys₆; Cys₅ and Cys₁₃; are connected by disulfide bonds; and wherein Cth₂ and Cys₁₀ are connected by a thioether bond; wherein the peptide has an acetylated N-terminus; and an unmodified C-terminus; emulsifiers; a mixture of aqueous and non-aqueous liquids; and a propellant.

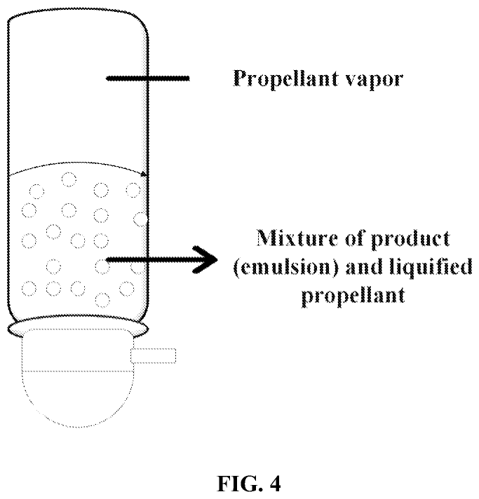

The pharmaceutical rectal foam composition was packaged in a pressurized canister in the form of an emulsion, in which the liquefied propellant is present as a mixture with the emulsion. A portion of the liquefied propellant equilibrates to form a vapor phase in the headspace of the container, as shown in FIG. 4. For rectal administration the canister is intended to be used in inverted orientation. When the actuator on the canister is depressed, the valve opens. The propellant in the vapor phase forces the mixture of emulsion and liquefied propellant through the opened valve into the metered dome. When the actuator is released, the contents in the metered dome are expelled from the canister. Following the expulsion from the canister, the liquefied propellant evaporates into a gas that is trapped forming a foam.

### Drug Substance

The physicochemical properties of the drug substance (peptide of the present disclosure) are summarized above; briefly, the drug substance is a 13 amino acid peptide with primary amino acid sequence Ac-Cys-Hcy-Glu-Leu-Cys-Cys-Asn-Val-Ala-Ala-Tyr-Gly-Cys-OH with an average molecular mass of 1401.6 Daltons.

The drug substance is slightly soluble (>1 mg/mL) in water and aqueous buffer at pH 7. The drug substance is minimally absorbed and is designed to act locally in the gastrointestinal tract. The peptide drug substance is stable up to 50-months with no appreciable change in purity or impurity profile when stored under frozen conditions (−20° C.). See Example 20 above.

### Excipients

The excipients used in the pharmaceutical rectal foam composition, 100 μg and 300 μg formulations were compendial, with the exception of Aeropin 35. All the excipients present in the final formulation are commonly used excipients in formulating emulsions and are below the maximum level allowed by the FDA Inactive Ingredient Database (IID) as shown in Table 45. A propellant gas mixture was selected to generate appropriate pressure within the container to expel the contents effectively in the form of a foam. The propellant mixture used is listed as an inactive ingredient in an FDA-approved rectal foam product.

The excipients included purified water, propylene glycol, sodium phosphate dibasic, sodium dihydrogen phosphate monohydrate, disodium edetate (disodium EDTA), methylparaben, light mineral oil, isopropyl myristate, white petrolatum, polyoxyl 20 cetostearyl ether, cetyl alcohol, stearyl alcohol, propylparaben and Aeropin 35. Aeropin 35 is a propellant gas system which is a mixture of n-butane, iso-butane, and propane.

TABLE 45
Summary information supporting safety of inactive ingredients, including IID levels.

| | | | | | IID |
|---|---|---|---|---|---|
| | Level in | IID | | | Maximum |
| | Peptide Rectal | Maximum | | IID Route of | Daily |
| Inactive Ingredients in IID/ | Emulsion per | Potency per | | Administration | Exposure |
| CAS Numbers | Dose (mg) ^{a} | unit dose | | and Dosage Form | (mg) |
| Propylene Glycol/57556^{b} | 104 | Unknown | | Rectal Aerosol | 1214 |
| | (9.01% w/w) | | | Foam | |
| Sodium Phosphate Dibasic/ | 1.70 | 0.47% | w/v | Topical Solution | — |
| 7558794 | (0.148% w/w) | | | | |
| Sodium Dihydrogen | 1.21 | 31.72 | mg | Oral Tablet | — |
| Phosphate Monohydrate/ | (0.105% w/w) | | | | |
| 10049215 | | | | | |
| Disodium EDTA/6381926^{b} | 0.518 | Unknown | | Rectal Aerosol | 2 |
| | (0.045% w/w) | | | Foam | |
| Methylparaben/99763 | 1.04 | 0.23% | w/v | Rectal Suspension | — |
| | (0.0901% w/w) | | | | |
| Light Mineral Oil/ | 62.2 | 1464.9 | mg | Vaginal | — |
| 92062356 | (5.41% w/w) | | | Suppository | |
| Isopropyl Myristate/110270 | 5.18 | 1% | w/w | Vaginal Cream | — |
| | (0.451% w/w) | | | | |
| White Petrolatum/8009038 | 10.4 | Unknown | | Topical Aerosol | 72 |
| | (0.901% w/w) | | | Foam | |
| Polyoxyl 20 Cetostearyl | 25.9 | Unknown | | Topical Aerosol | 374 |
| Ether/68439496 | (2.25% w/w) | | | Foam | |
| Cetyl Alcohol/36653824 | 10.4 | Unknown | | Rectal Aerosol | 17 |
| | (0.901% w/w) | | | Foam | |
| Stearyl Alcohol/67762270 | 10.4 | Unknown | | Topical Aerosol | 19 |
| | (0.901% w/w) | | | Foam | |
| Propylparaben/94133 | 0.207 | 0.03% | w/v | Rectal Suspension | — |

| | | | | |
|---|---|---|---|---|
| | (0.0180% w/w) | | | |
| Propane/74986^{bc} | 5.7 to 10.3 | Unknown | Rectal Aerosol | 9 |
| | | | Foam | |
| n-Butane/106978^{bc} | 5.7 to 10.3 | Unknown | Rectal Aerosol | 12 |
| | | | Foam | |
| Isobutane/75285^{bc} | 94.6 to 101 | Unknown | Rectal Aerosol | 142 |
| | | | Foam | |

^{a} Dosing is once daily. Therefore, a single dose equals maximum daily exposure.
^{b}These excipients have been used in FDA approved Rectal foam product.
^{c}The propellant used in pharmaceutical rectal foam composition is a mixture of propane (5-9%), isobutane (83-89%), and n-butane (5-9%). A similar combination has been used in an FDA approved rectal foam.

### Drug Product

### Formulation Development

Rectal administration utilizing a foam or enema formulation is the most efficient method for ensuring colonic delivery of an early-stage development product. The pharmaceutical composition in enema form, used in Phase 1 studies, was formulated in a manner to administer a volume comparable to the minimum possible expansion volume of the rectal foam in the present example.

Compared to a conventional enema, a stable (hard to collapse) type of rectal foam offers the added advantages of prolonged residence time, reduction of leakage from the rectum, and ease of self-administration. A stable foam is a type of foam which does not collapse/break immediately upon application.

The pharmaceutical rectal foam composition operable to deliver 100 μg and 300 μg of the active ingredient, will be formulated as a stable foam and will be utilized for the Phase 2 study. The pharmaceutical rectal foam composition is formulated as an oil-in-water emulsion. The emulsion contains aqueous and oil components, pH adjusting salts, chelating agent, preservatives, emulsifier, and stiffening (viscosity building) agent. To avoid any irritation to rectal or colon lining, the pH of the drug product is targeted to match the rectal and colon pH.

The emulsion is filled into a pressurized canister along with a gas propellant to deliver the product as a foam. Upon expulsion from the canister, the foam does not collapse quickly and expands to a volume of not less than 20 mL. The delayed collapse and large volume of expansion is beneficial to allow maximum exposure of the drug to the colon.

The drug product is designed to deliver a single dose in a single actuation. Each drug product canister is a multidose unit and is intended to be used for 22 actuations, one priming actuation followed by 21 dosing actuations. Each can contain 34.4 g of drug product. The rectal foam is formulated in two configurations such that a single actuation of 1.15 g of foam corresponds to a unit dose of 100 μg or 300 μg. By instructing that each canister only be actuated 21 times ensures that each actuation weight is accurate.

Initial foam formulation prototypes were designed to provide desired foam characteristics. A prototype development stability study was conducted by bracketing the clinical doses (30 μg to 3000 μg) at 5° C./Ambient RH and 25° C./Ambient RH for 6 months. This bracketing approach was employed since it was not known earlier what dose would be chosen for the Phase 2 clinical trial. The doses, 30 μg and 3000 μg, were selected as the extremes since they covered the range of potential strengths to be tested in the Phase 1 single-ascending dose (SAD) and multiple-ascending dose (MAD) clinical studies.

All dose strengths that fall within these extremes have closely related formulations-emulsions with differing amounts of drug substance, but identical quantities of the same excipients, and the same container closure system. In accordance with the FDA Guidance for Industry—Q1D Bracketing and Matrixing Designs for Stability Testing of New Drug Substances and Products, if the stability of the extremes is shown to be different, the intermediates should be considered no more stable than the least stable extreme (i.e., the shelf life for the intermediates should not exceed that for the least stable extreme). The doses ultimately selected for testing in the SAD study were 100 μg, 300 μg, 900 μg, and 2500 μg. Based on the results of the SAD study, the 100 μg and 300 μg doses were selected for inclusion in the MAD study. It is anticipated that both the 100 μg and 300 μg doses will also be included in the Phase 2 clinical study to be conducted with the rectal foam; therefore, stability information from 30 μg and 3000 μg can be leveraged for 100 and 300 μg. The results of the prototype development stability study are shown in Table 46 and Table 47.

TABLE 46
Stability study results at 5° C./Ambient RH for Pharmaceutical
rectal foam composition, 30 μg and 3000 μg.

| | | | | | | | |
|---|---|---|---|---|---|---|---|
| Parameters | Acceptance Criteria | — | Bulk | T0 | T1 | T3 | T6 |

1797-1111R01, 30 μg

| Specific Gravity of | Report Results | | — | 0.997 | — | — | — | — |
|---|---|---|---|---|---|---|---|---|
| Bulk | | | | | | | | |
| pH (Foam) | 6.5 to 7.5 | | — | 7.1 | 7.0 | 6.9 | 7.0 | 7.0 |
| Foam Height (ml) | ≥20 | mL | Mean | — | 28 | 25 | 26 | 26 |
| | | | (n = 3) | | | | | |
| Duration of | ≥1 | minute | Mean | — | 1 | 1 | 1 | 1 |

| | | | | | | | |
|---|---|---|---|---|---|---|---|
| Expansion (min) | | (n = 3) | | | | | |
| Weight Per | Report Results | Mean | — | 1.30 | 1.20 | 1.30 | 1.28 |
| Actuation (g) | | (n = 3) | | | | | |
| Appearance (White | White to off-white | — | — | Pass | Pass | Pass | Pass |
| Foam) | foam | | | | | | |
| Peptide | 90%-110% of label | Mean | 100.6 | 101.9 | 100.4 | 101.4 | 101.3 |
| | claim | (n = 10) | | | | | |
| | | % RSD | 0.37 | 0.80 | 1.64 | 0.53 | 0.31 |
| Methylparaben | 90%-110% of label | Mean | 100.9 | 98 | 100.2 | 98.9 | 100 |
| | claim | (n = 10) | | | | | |
| | | % RSD | 0.34 | 1.42 | 0.80 | 0.37 | 0.53 |
| Propylparaben | 90%-110% of label | Mean | 100.9 | 98.9 | 100.9 | 98.8 | 100.4 |
| | claim | (n = 10) | | | | | |
| | | % RSD | 0.39 | 1.41 | 0.70 | 0.24 | 0.47 |

1797-1111R02, 3000 μg

| Specific Gravity of | Report Results | | — | 0.987 | — | — | — | — |
|---|---|---|---|---|---|---|---|---|
| Bulk | | | | | | | | |
| pH (Foam) | 6.5 to 7.5 | | — | 6.8 | 6.9 | 6.8 | 6.9 | — |
| Foam Height (ml) | ≥20 | mL | Mean | — | 30 | 24 | 27 | 30 |
| | | | (n = 3) | | | | | |
| Duration of | ≥1 | minute | Mean | — | 1.25 | 1.00 | 1.00 | 1.00 |

| | | | | | | | |
|---|---|---|---|---|---|---|---|
| Expansion (min) | | (n = 3) | | | | | |
| Weight Per | Report Results | Mean | — | 1.36 | 1.25 | 1.26 | 1.46 |
| Actuation (g) | | (n = 3) | | | | | |
| Appearance (White | White to off-white | — | — | Pass | Pass | Pass | Pass |
| Foam) | foam | | | | | | |
| Peptide | 90%-110% of label | Mean | 92.2 | 97.5 | 97.4 | 98.3 | 96.9 |
| | claim | (n = 10) | | | | | |
| | | % RSD | 0.41 | 1.53 | 0.88 | 1.29 | 1 |
| Methylparaben | 90%-110% of label | Mean | 98.5 | 99.6 | 99.5 | 101.4 | 98.8 |
| | claim | (n = 10) | | | | | |

1797-1111R01, 30 μg

| | | | | | | | |
|---|---|---|---|---|---|---|---|
| | | % RSD | 0.27 | 0.75 | 0.93 | 0.75 | 0.45 |
| Propylparaben | 90%-110% of label | Mean | 98.5 | 100.4 | 100 | 101.2 | 99 |
| | claim | (n = 10) | | | | | |
| | | % RSD | 0.25 | 0.79 | 1 | 0.66 | 0.38 |

TABLE 47
Stability study results at 25° C./Ambient RH for Pharmaceutical
rectal foam composition, 30 μg and 3000 μg.

| | | | | | | | |
|---|---|---|---|---|---|---|---|
| Parameters | Acceptance Criteria | — | Bulk | T0 | T1 | T3 | T6 |

1797-1111R01, 30 μg

| Specific Gravity of | Report Results | | — | 0.997 | — | — | — | — |
|---|---|---|---|---|---|---|---|---|
| Bulk | | | | | | | | |
| pH (Foam) | 6.5 to 7.5 | | — | 7.1 | 7.0 | 7.2 | 7.3 | 7.1 |
| Foam Height (ml) | ≥20 | mL | Mean | — | 28 | 27 | 23 | 25 |
| | | | (n = 3) | | | | | |
| Duration of | ≥1 | minute | Mean | — | 1.0 | 0.75 | 1.0 | 1.0 |

| | | | | | | | |
|---|---|---|---|---|---|---|---|
| Expansion (min) | | (n = 3) | | | | | |
| Weight Per | Report Results | Mean | — | 1.30 | 1.25 | 1.25 | 1.03 |
| Actuation (g) | | (n = 3) | | | | | |
| Appearance (White | White to off- | — | — | Pass | Pass | Pass | Pass |
| Foam) | white foam | | | | | | |
| Peptide | 90%-110% of label | Mean | 100.6 | 101.9 | 96.4 | 97.7 | 94.6 |
| | claim | (n = 10) | | | | | |
| | | % RSD | 0.37 | 0.80 | 2.86 | 0.87 | 1.42 |
| Methylparaben | 90%-110% of label | Mean | 100.9 | 98 | 99.9 | 98.3 | 98.1 |
| | claim | (n = 10) | | | | | |
| | | % RSD | 0.34 | 1.42 | 0.81 | 1.1 | 0.46 |
| Propylparaben | 90%-110% of label | Mean | 100.9 | 98.9 | 100.7 | 99.9 | 98.6 |
| | claim | (n = 10) | | | | | |
| | | % RSD | 0.39 | 1.41 | 0.65 | 1.1 | 0.54 |

1797-1111R02, 3000 μg

| Specific Gravity of | Report Results | | — | 0.987 | — | — | — | — |
|---|---|---|---|---|---|---|---|---|
| Bulk | | | | | | | | |
| pH (Foam) | 6.5 to 7.5 | | — | 6.8 | 6.9 | 6.8 | 7.1 | 6.9 |
| Foam Height (ml) | ≥20 | mL | Mean | — | 30 | 26 | 23 | 27 |
| | | | (n = 3) | | | | | |
| Duration of | ≥1 | minute | Mean | — | 1.25 | 1.00 | 1.00 | 1.00 |

| | |
|---|---|
| Expansion (min) | (n = 3) |

1797-1111R01, 30 μg

| | | | | | | | |
|---|---|---|---|---|---|---|---|
| Weight Per | Report Results | Mean | — | 1.36 | 1.35 | 1.16 | 1.27 |
| Actuation (g) | | (n = 3) | | | | | |
| Appearance (White | White to off-white | — | — | Pass | Pass | Pass | Pass |
| Foam) | foam | | | | | | |
| Peptide | 90%-110% of label | Mean | 92.2 | 97.5 | 93.5 | 90.0 | 86.3 |
| | claim | (n = 10) | | | | | |
| | | % RSD | 0.41 | 1.53 | 0.28 | 0.37 | 1.76 |
| Methylparaben | 90%-110% of label | Mean | 98.5 | 99.6 | 98.3 | 97.9 | 97.4 |
| | claim | (n = 10) | | | | | |
| | | % RSD | 0.27 | 0.75 | 0.68 | 0.65 | 0.58 |
| Propylparaben | 90%-110% of label | Mean | 98.5 | 100.4 | 98.9 | 99.4 | 97.6 |
| | claim | (n = 10) | | | | | |
| | | % RSD | 0.25 | 0.79 | 0.56 | 0.63 | 0.54 |

Due to the drop in assay (below 90%) observed at the end of 6 months at 25° C./Ambient RH, it is recommended to store the pharmaceutical rectal foam composition at 2-8° C. until the time of use. The acceptable assay results at the end of 1 month at 25° C./Ambient RH support the storage of a canister at room temperature for at most one month after it is removed from 2-8° C. and during the time of use.

The formulation composition of pharmaceutical rectal foam composition 100 μg and 300 μg is identical to Placebo to Match (PTM), Rectal Foam except for a very low dose of drug substance added to it. The filling/packaging process is also identical to PTM Rectal foam. Therefore, the data shown in PTM Rectal Foam to understand the trend of dispensed amount per actuation within and across different canisters is representative of pharmaceutical rectal foam composition 100 μg and 300 μg.

### Manufacturing Process Development

The pharmaceutical rectal foam composition, 100 μg and 300 μg, manufacturing process can be subdivided into two main steps: (1) preparation of emulsion, and (2) filling of the emulsion into canisters. For smaller batch sizes, both steps were performed using manual procedures while the larger batch sizes were performed on a semiautomatic production line.

Preparation of emulsion involves a series of mixing steps resulting in an oil-in-water emulsion, with the peptide of the present disclosure (drug substance) dissolved in the aqueous continuous phase. During the preparation step (compounding), the emulsion base is heated to between 75-80° C. to solubilize the solid components prior to the addition of the drug substance. To minimize the risk of the drug substance degrading at high temperature, the emulsion base is cooled down to below 30° C. before addition of the drug substance to obtain the peptide bulk emulsion.

Following preparation of the peptide bulk emulsion, the emulsion is filled in clean canisters. The bulk emulsion is dispensed into empty canisters and the valve is crimped into place. The propellant mixture is injected into the canister through the valve. The foam shield and metering head are fixed into place on the canister. During the filling process, the canisters are sampled to check emulsion net fill weight, propellant net fill weight, crimp dimension, and pressure within the canister. All the canisters pass through a water bath leak detector to ensure there are no cans with leaks.

### Container Closure System

The pharmaceutical rectal foam composition, 100 μg and 300 μg, is presented as a multidose unit contained in an aluminum aerosol canister fitted with a 1-inch valve, foam shield, and a 1.35 mL metered head actuator. The canister is individually packaged in a carton. Each carton is supplied to the patient with a kit that contains 21 single-use pre-lubricated applicator tubes and 21 individual disposal bags. All the primary packaging components comply with federal requirements for drug product containers and closures per 21 CFR Part 211.94. All the primary packaging components that could reasonably come into contact with peptide are certified to comply with current federal requirements for food contact regulations 21 CFR Parts 172-186.

### Microbiological Attributes

The pharmaceutical rectal foam composition, 100 μg and 300 μg, is a nonsterile formulation intended for rectal administration. Microbial limit testing per USP <61> and test for Burkholderia cepacia Complex (BCC) per USP<60> will be conducted at release and on stability to evaluate the presence and potential of microbial growth. In addition, antimicrobial effectiveness testing per USP <51> will be conducted at release and on stability to evaluate the effectiveness of preservatives.

The pharmaceutical rectal foam composition, 100 μg and 300 μg, is a multidose canister intended to be used for 21 doses. During storage and patient administration, there is no risk for ingress of microbial organisms in the product due to: (1) the presence of a valve ensures that all the doses remain contained within the canister until the time of administration; (2) the canister does not come in direct contact with the site of administration because the dosage form is administered via a single use rectal applicator tube; and (3) the canister is a pressurized container, and the contents inside the canister are maintained under positive pressure, providing no opportunity for any ingress of contamination.

### Example 22. Batch Formula: Rectal Foam

The quantitative batch formula for manufacturing of the pharmaceutical rectal foam composition, 100 μg and 300 μg, is provided in Table 48.

TABLE 48
Quantitative composition of the pharmaceutical rectal
foam composition, 100 μg and 300 μg.

| | Theoretical Quantity per | | |
|---|---|---|---|
| | 41.0 kg Batch (kg)^{a} | | |
| | | | Quality |
| Component | 100 μg | 300 μg | Standard |
| Peptide^{b} | 0.00396 | 0.0119 | In-House |
| Purified water | 31.8 | 31.8 | USP |
| Propylene glycol | 4.10 | 4.10 | USP |
| Dibasic sodium phosphate | 0.0672 | 0.0672 | USP |
| Sodium dihydrogen phosphate | 0.0478 | 0.0478 | USP |
| monohydrate | | | |
| Disodium EDTA | 0.0205 | 0.0205 | USP |
| Methylparaben | 0.0410 | 0.0410 | NF |
| Light mineral oil | 2.46 | 2.46 | NF |
| Isopropyl myristate | 0.205 | 0.205 | NF |
| White petrolatum | 0.410 | 0.410 | USP |
| Polyoxyl 20 cetostearyl ether | 1.03 | 1.03 | NF |
| Cetyl alcohol | 0.410 | 0.410 | NF |
| Stearyl alcohol | 0.410 | 0.410 | NF |
| Propyl paraben | 0.00820 | 0.00820 | NF |
| Total Bulk Emulsion | 41.0 | 41.0 | — |

^{a}Each canister is a multidose unit and contains 34.4 g of product. During filling operation 31.0 g of emulsion and 3.4 g of propellant (Aeropin 35) is filled into each canister.
^{b}The actual quantity of peptide of the present disclosure (drug substance) is corrected based on the potency of the drug substance with a concomitant adjustment of Purified Water.

### Example 23. Description of Manufacturing Process and Processing Controls

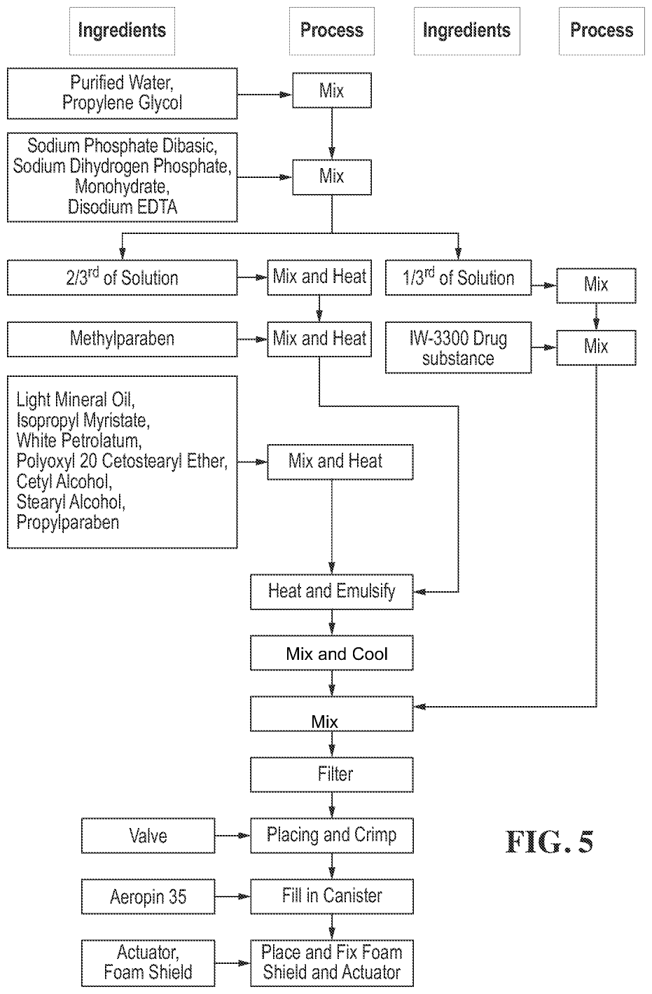

The pharmaceutical rectal foam composition, 100 μg and 300 μg, is manufactured using the process illustrated by the flow diagram shown in FIG. 5.

The pharmaceutical rectal foam composition, 100 μg and 300 μg, is prepared by an emulsification process. The manufacturing process comprises a series of low shear mixing steps to form the emulsion, which is then packaged into an aerosol canister. The steps are as follows:

Step 1—Preparation of aqueous phase: Purified water and propylene glycol are added to a stainless-steel vessel and mixed using an overhead mixer. To this mixture, sodium phosphate, sodium dihydrogen phosphate monohydrate, and disodium EDTA are added and continuously mixed until dissolved. At this stage, approximately one third portion of this aqueous solution is removed and kept aside in a separate vessel for preparation of the active phase (step 4). The remaining two thirds of the aqueous solution is heated to between 75° C. and 80° C. while mixing to aid solubilization of methylparaben. Methylparaben is added and mixed until dissolved. This clear solution is the aqueous phase.

Step 2—Preparation of oil phase: Light mineral oil, isopropyl myristate, white petrolatum, polyoxyl 20 cetostearyl ether, cetyl alcohol, stearyl alcohol and propylparaben are added to another stainless-steel vessel and mixed using an overhead mixer. The mixture is heated to between 75° C. and 80° C. to aid solubilization of solid components. Mixing while heating is continued until no undissolved solids are seen. This solution is the oil phase.

Step 3—Emulsification: The oil phase (dispersed phase) followed by a hot water rinse of the vessel containing the oil phase is added to the aqueous phase (continuous phase) under continuous mixing while maintaining the heat between 75° C. and 80° C. The mixture is then cooled to 30° C. or below until a white to off-white thin liquid emulsion is formed. This is the base emulsion.

Step 4—Preparation of active phase: Water content result is used to correct the amount of drug substance charged during the manufacture of peptide bulk emulsion. To the segregated one third portion of aqueous solution (refer Step 1), peptide of the present disclosure (drug substance) is added under continuous mixing followed by water rinse of the drug substance container. Mixing is continued until no undissolved solids are seen. This is the active phase.

Step 5—Addition of active phase to prepared base emulsion: The active phase is then added to the base emulsion which is cooled to 30° C. or below, under continuous mixing. The resultant is the bulk emulsion which is filtered during transfer to a holding vessel.

Step 6—In-process check: The bulk emulsion is evaluated for appearance, pH, and specific gravity.

Step 7—Filling in aerosol canister: A target quantity of bulk emulsion (31.0 g) is filled into each canister. The valves are positioned and crimped with vacuum applied onto the filled canisters. A vacuum check is performed. Then a target amount of propellant (3.4 g) under pressure is introduced through the valve into the canisters. The foam shield and metered head actuator are positioned and fixed on the canisters. Routine in-process checks are performed along the filling line (bulk emulsion fill weights, propellant fill weights, valve crimping dimensions, pressure within the canisters) at pre-defined intervals to ensure consistent performance of the filling equipment and process over the entire filling run. All the canisters are tested for leaks by passing through a hot water bath, maintained at a temperature range of 130° F. to 138° F., for a minimum of 1 minute. The acceptable canisters are labelled with a lot number. The canisters are stored at 2° C. to 8° C.

Step 8—Clinical Packaging and Labeling: The canister is individually packaged in a carton. Each carton is supplied to the subject in need thereof with a kit that contains 21 single-use pre-lubricated applicator tubes and 21 individual disposal bags. The carton containing the canister is stored at 2° C. to 8° C. The kit containing applicator tubes and disposal bags is stored at ambient conditions.

### In Process Controls

Table 49 below provides a summary of the manufacturing in-process controls for the pharmaceutical rectal foam composition.

TABLE 49
In-process controls for the pharmaceutical rectal
foam composition, 100 μg and 300 μg.

| Test | Method | Acceptance Criteria |
|---|---|---|
| Appearance | Visual | White to off-white thin liquid emulsion |
| pH | USP <791> | 6.5 to 7.5 |
| Specific Gravity | USP <841> | Report Result |

### Example 24. Informal Stability of Medicated Foam

The present example addressed the informal stability and evaluation for the pharmaceutical rectal foam compositions, Prototypes A and B. Here, two foam Prototypes were developed allowing delivery of a minimum of 30 μg and a maximum of 3,000 μg of the peptide of the present disclosure in a volume suitable for adult administration. This IND product is for the treatment of lower colon and rectal pain and will be packaged in a 30-g aluminum aerosol can with a metered dose valve. This example covers the informal stability for Prototypes A and B and the formulation enhancement work to Prototype A, which became the final Prototype for further consideration.

### Prototype A

### Aqueous Phase

The Aqueous Phase was prepared in a glass beaker into which Purified Water and Propylene Glycol were added. This was placed under an overhead lab mixer with a 3-inch 3-prong mixing blade and mixed at room temperature conditions. Sodium Phosphate, Sodium Dihydrogen Phosphate and Disodium EDTA were added. Mixing continued at 346-400 RPM until all solids were visually dissolved which took about 8-15 minutes. A sample was removed to perform an in-process pH check, where the pH was to be between 6.5-7.5. Approximately ⅓ of the Aqueous Phase was removed to prepare the Active Phase. The remaining ⅔ of the Aqueous Phase was heated to 75-80° C.

Methylparaben was added and mixed at 346-400 RPM until all solids were visually dissolved which took 10 minutes while maintaining 75-80° C. The phase was observed to be a uniform, clear solution with no solid particles present.

### Oil Phase

The Oil Phase was prepared in a glass beaker into which the Oil Phase ingredients; Light Mineral Oil, Isopropyl Myristate, White Petrolatum, Polyoxyl 20 Cetostearyl Ether, Cetyl Alcohol, Stearyl Alcohol and Propylparaben were added. This was placed under an overhead lab mixer with a 1.5-inch 3-prong mixing blade and was mixed at 600-800 RPM while heating to 75-80° C. and until all waxy solids were observed as melted. The phase was observed to be a uniform solution with no solid particles or un-melted waxy particles present.

### Emulsification Phase

The Oil Phase was added to the Aqueous Phase. Mixing continued at 400-600 RPM with a 3-inch 3-prong mixing blade until visually uniform for about 10-20 minutes while maintaining 75-80° C. This was then cooled to 30° C. or below. The phase was observed to be a uniform, thin white to off-white milky cream emulsion.

### Active Phase

The Active Phase was prepared with ⅓ Aqueous Phase from above at room temperature where the peptide of the present disclosure (Active Pharmaceutical Ingredient (API)) adjusted for potency was added. The potency for the API content in Lot B01511P023 used was 91.2%. For the Placebo batch, no API was added. Mixing continued with a ½ inch 3 prong mixing blade at 600-1040 RPM until all solids were visually dissolved which took from 4-22 minutes. The Active Phase was added to the base emulsion. Mixing continued with a 3-inch 3 prong mixing blade at 500 RPM until visually uniform around 10-15 minutes. The final pH check was in the range 6.5-7.5. The final batch was observed to be a uniform, thin white to off-white milky cream emulsion.

### Packaging

For each of the following batches of Prototype A, 31 g of bulk product was manually filled with a 60-mL syringe into 35 mm×64 mm cans with a PPG liner. Each canister was hand crimped with a 1-inch valve. AP-70 propellant was added at 1.8-2.4 g (target: 2.1 g) per can. The amount of propellant was based on similar foam products to be evaluated and would be adjusted based on performance enhancement studies. These were then manually capped with the foam shied and 1.35 mL metering head. Cans of Prototype A were placed at 5° C. and 25° C. for informal stability.

The following Table 50 provides the lab batch Lots for Prototype A. For the two Active formulations, the amount of Purified Water USP/EP was adjusted to compensate for the API concentration. For each lab batch, the manufacturing date is reflected within the Lot code. For example: Lot 1797-1109R02 was manufactured on Nov. 9, 2020, and so on. Package dates for each lab batch was not recorded but these were typically packaged on the day of manufacturer or the next day.

TABLE 50
Prototype A lab batches for informal stability bulk formulations.

| | Lot 1797-1109R02 | | | Lot 1797-1111R01 | | | Lot 1797-1111R02 | | |
|---|---|---|---|---|---|---|---|---|---|
| | Placebo | | | 30 μg/dose | | | 3000 μg/dose | | |
| | | Batch | per | | Batch | per | | Batch | per |
| | | Amounts | Actuation | | Amounts | Actuation | % | Amounts | Actuation |
| Ingredient | % w/w | (g) | (pg) | % w/w | (g) | (pg) | w/w | (g) | (pg) |
| Purified Water | 77.5495 | 1085.69 | 893766 | 77.5469 | 775.47 | 893738 | 77.2892 | 541.03 | 890766 |
| USP/EP | | | | | | | | | |
| Propylene | 10.0000 | 140.01 | 115251 | 10.0000 | 100.01 | 115251 | 10.0000 | 70.00 | 115251 |
| Glycol USP | | | | | | | | | |
| Sodium Phosphate | 0.1640 | 2.30 | 1890 | 0.1640 | 1.64 | 1890 | 0.1640 | 1.15 | 1890 |
| Dibasic USP | | | | | | | | | |
| Sodium | 0.1165 | 1.63 | 1343 | 0.1165 | 1.17 | 1343 | 0.1165 | 0.8159 | 1343 |
| Dihydrogen | | | | | | | | | |
| Phosphate | | | | | | | | | |
| Monohydrate USP | | | | | | | | | |
| Disodium EDTA | 0.0500 | 0.7022 | 576 | 0.0500 | 0.5001 | 576 | 0.0500 | 0.3509 | 576 |
| USP | | | | | | | | | |
| Methylparaben NF | 0.1000 | 1.40 | 1153 | 0.1000 | 1.00 | 1153 | 0.1000 | 0.7007 | 1153 |
| Light Mineral Oil | 6.0000 | 84.00 | 69151 | 6.0000 | 60.03 | 69151 | 6.0000 | 42.01 | 69151 |
| NF | | | | | | | | | |
| Isopropyl | 0.5000 | 7.01 | 5763 | 0.5000 | 5.01 | 5763 | 0.5000 | 3.50 | 5763 |
| Myristate NF | | | | | | | | | |
| White | 1.0000 | 14.17 | 11525 | 1.0000 | 10.00 | 11525 | 1.0000 | 7.03 | 11525 |
| Petrolatum | | | | | | | | | |
| USP | | | | | | | | | |
| Polyoxyl 20 | 2.5000 | 35.00 | 28813 | 2.5000 | 25.00 | 28813 | 2.5000 | 17.50 | 28813 |
| Cetostearyl Ether NF | | | | | | | | | |
| Cetyl | 1.0000 | 14.00 | 11525 | 1.0000 | 10.01 | 11525 | 1.0000 | 7.00 | 11525 |
| Alcohol NF | | | | | | | | | |
| Stearyl Alcohol NF | 1.0000 | 14.00 | 11525 | 1.0000 | 10.00 | 11525 | 1.0000 | 7.00 | 11525 |
| Propylparaben NF | 0.0200 | 0.2804 | 231 | 0.0200 | 0.2023 | 231 | 0.0200 | 0.1409 | 231 |
| API (adj for | | | | 0.0026 | 0.0269 | 30 | 0.2603 | 1.7600 | 3000 |
| potency) | | | | | | | | | |
| Total | 100.00 | 1400 | | 100.00 | 1000 | | 100.00 | 700 | |
| AP-70 | | 2.1 g | 77490 | | 2.1 g | 77490 | | 2.1 g | 77490 |
| Propellant | | | | | | | | | |
| Total | | | 1230002 | | | 1230004 | | | 1230002 |

### Prototype B

### Aqueous Phase

The Aqueous Phase was prepared in a glass beaker into which Purified Water was added. This was placed under an overhead lab mixer with a 3-inch 3-prong mixing blade. This was mixed at room temperature conditions while Sodium Phosphate, Sodium Dihydrogen Phosphate and Disodium EDTA were added. Mixing continued at 292-380 RPM until all solids were visually dissolved which took 3-17 minutes. A sample was removed to perform an in-process pH check, where the pH was to be between 6.5-7.5. Approximately ⅓ of the Aqueous Phase was removed to prepare the Active Phase. The remaining ⅔ was heated to 68-73° C. Methylparaben was added and mixed at 292-380 RPM for 8-10 minutes and until all solids were visually dissolved while maintaining 68-73° C. The phase was a uniform, clear solution with no solid particles observed.

### Oil Phase

The Oil Phase was prepared in a glass beaker into which the Oil Phase ingredients; Propylene Glycol, Cetyl Alcohol, Emulsifying Wax, Polyoxylene 10 Stearyl Ether and Propylparaben were added. This was placed under an overhead lab mixer with a 1.5-inch 3-prong mixing blade and was mixed 600-960 RPM while heating to 68-73° C. and until all waxy solids were observed as melted. The phase was a uniform solution with no solid particles or un-melted waxy particles observed.

### Emulsification Phase

Oil Phase was added to the Aqueous Phase. Mixing continued at 400-607 RPM with a 3-inch 3-prong mixing blade for 10-17 minutes and until visually uniform while maintaining 68-73° C. This was then cooled to 30° C. or below. The final temperature was 30° C. or below. The phase was observed to be a uniform, thin white to off-white milky cream emulsion.

### Active Phase

The Active Phase was prepared with ⅓ Aqueous Phase from above at room temperature where the peptide of the present disclosure (Active Pharmaceutical Ingredient (API)) adjusted for potency was added. The potency for the API content in Lot B01511P023 used was 91.2%. For the Placebo batch, no API was added. Mixing continued at 600-880 RPM for 5-12 minutes and until all solids were visually dissolved. The Active Phase was added to the base emulsion. Mixing continued with a 3-inch 3 prong mixing blade at 400-600 RPM for 10-15 minutes and until visually uniform. The final pH check was in the range 6.5-7.5. The final batch was observed to be a uniform, thin white to off-white milky cream emulsion.

### Packaging

For each of the following batches of Prototype B, 31 g of bulk product was manually filled using a 60-ml syringe into 35 mm×64 mm cans with a PPG liner. Each canister was hand crimped with a 1-inch valve. AP-70 propellant was added at 1.8-2.4 g (Target: 2.1 g) per can. The amount of propellant was based on similar foam products to be evaluated and would be adjusted based on performance enhancement studies. These were then manually capped with the foam shied and 1.35 mL metering head. Cans of Prototype B were placed at 5° C. and 25° C. for informal stability.

The following Table 51 provides the lab batch Lots for Prototype B. For the two Active formulations, the amount of Purified Water USP/EP was adjusted to compensate for the API concentration. For each lab batch, the manufacturing date is reflected within the Lot code. For example: Lot 1797-1109R03 was manufactured on Nov. 9, 2020, and so on. Package dates for each lab batch was not recorded but these were typically packaged on the day of manufacturer or the next day.

TABLE 51
Prototype B lab batches for informal stability bulk formulations.

| | Lot 1797-1109R03 | | | Lot 1797-1110R01 | | | Lot 1797-1110R02 | | |
|---|---|---|---|---|---|---|---|---|---|
| | Placebo | | | 30 μg/dose | | | 3000 μg/dose | | |
| | | | per | | | per | | | per |
| | | Amounts | Actuation | | Amounts | Actuation | | Amounts | Actuation |
| Ingredient | % w/w | (g) | (Pg) | % w/w | (g) | (Pg) | % w/w | (g) | (Pg) |
| Propylene Glycol | 16.4800 | 230.73 | 189934 | 16.4800 | 164.84 | 189934 | 16.4800 | 115.36 | 189934 |
| USP/EP | | | | | | | | | |
| Cetyl Alcohol NF | 0.7000 | 9.80 | 8068 | 0.7000 | 7.01 | 8068 | 0.7000 | 4.90 | 8068 |
| Emulsifying Wax | 1.4000 | 19.62 | 16135 | 1.4000 | 14.00 | 16135 | 1.4000 | 9.80 | 16135 |
| NF | | | | | | | | | |
| Propylparaben NF | 0.0200 | 0.2830 | 231 | 0.0200 | 0.1998 | 231 | 0.0200 | 0.1418 | 231 |
| Polyoxylene (10) Stearyl | 1.4000 | 19.61 | 16135 | 1.4000 | 14.03 | 16135 | 1.4000 | 9.80 | 16135 |
| Ether NF | | | | | | | | | |
| Purified Water | 79.5695 | 1113.97 | 917046 | 79.5669 | 795.70 | 917016 | 79.3092 | 556.99 | 914046 |
| USP/EP | | | | | | | | | |
| Sodium Dihydrogen | 0.1165 | 1.63 | 1343 | 0.1165 | 1.17 | 1343 | 0.1165 | 0.8160 | 1343 |
| Phosphate | | | | | | | | | |
| Monohydrate USP | | | | | | | | | |
| Sodium Phosphate | 0.1640 | 2.30 | 1890 | 0.1640 | 1.64 | 1890 | 0.1640 | 1.15 | 1890 |
| Dibasic USP | | | | | | | | | |
| Disodium EDTA | 0.0500 | 0.7000 | 576 | 0.0500 | 0.4992 | 576 | 0.0500 | 0.3501 | 576 |
| USP | | | | | | | | | |
| Methylparaben NF | 0.1000 | 1.41 | 1153 | 0.1000 | 1.01 | 1153 | 0.1000 | 0.7007 | 1153 |
| API (adj for potency) | | | | 0.002603 | 0.0270 | 30 | 0.260300 | 1.82 | 3000 |
| Total | 100.00 | 1400 | | 100.00 | 1000 | | 100.00 | 1702 | |
| AP-70 Propellant | | 2.1 g | 77490 | | 2.1 g | 77490 | | 2.1 g | 77490 |
| Total | | | 1230001 | | | 1230001 | | | 1230001 |

The following tables provide the informal stability physical and analytical results for the lots produced for Prototypes A and B. Cans of Prototypes A and B were placed on informal stability at 5° C. and 25° C. and tested at the following time points.

TABLE 52
Prototype A information stability results. Placebo physical results.
1797-1109R02 - Prototype A Placebo Physical Results

| | | Initial | Initial | 1 Mo @ | 1 Mo @ | 2 Mo @ | 3 Mo @ | 3 Mo @ | 6 Mo @ | 6 Mo @ | |
|---|---|---|---|---|---|---|---|---|---|---|---|
| | | (bulk) | (Foam) | 5 C. | 25 C. | 25 C. | 5 C. | 25 C. | 5 C. | 25 C. | Mean |
| | | Date | | | | | | | | | |
| | | Nov. 7, 2020 | | Dec. 17, 2020 | | Jan. 16, 2021 | Feb. 15, 2021 | | May 16, 2021 | | |
| Specific Gravity | | 0.983 | | | | | | | | | |
| pH | 6.5-7.5 | 7.1 | 7.0 | 6.8 | 6.9 | 7.3 | 6.9 | 7.0 | 7.0 | 6.9 | 7.0 |
| Foam | Sample 1 | | 20 | 26 | 19 | 17 | 28 | 29 | 25 | 24 | 24 |
| Height | Sample 2 | | 29 | 28 | 28 | 11 | 26 | 25 | 26 | 27 | |
| (mL) | Sample 3 | | 24 | 27 | 25 | 22 | 26 | 23 | 24 | 26 | RSD = 17.1% |
| | | Average | 24 | 27 | 24 | 17 | 27 | 26 | 25 | 26 | |
| | | % RSD | 18.5 | 3.7 | 19.1 | 33.0 | 4.3 | 11.9 | 4.0 | 6.0 | |
| Duration of | Sample 1 | | 1 | 1 | 1 | 1 | 1 | 1 | 1 | 1 | 1 |
| Expansion | Sample 2 | | 1 | 1 | 1 | 1 | 1 | 1 | 1 | 1 | |
| (min) | Sample 3 | | 1 | 1.5 | 1 | 1 | 1 | 1 | 1 | 1 | RSD = 10.0% |
| | | Average | 1 | 1 | 1 | 1 | 1 | 1 | 1 | 1 | |
| | | % RSD | 0.0 | 24.7 | 0.0 | 0.0 | 0.0 | 0.0 | 0.0 | 0.0 | |
| Weight | Sample 1 | | 1.18 | 1.51 | 1.27 | 1.15 | 1.34 | 1.19 | 1.28 | 1.17 | 1.22 |
| per | Sample 2 | | 1.33 | 1.30 | 1.26 | 1.13 | 1.23 | 1.20 | 1.26 | 1.22 | |
| Actuation | Sample 3 | | 1.22 | 1.25 | 1.20 | 1.14 | 1.23 | 1.10 | 1.16 | 1.21 | RSD = 7.6% |
| (g) | Sample 4 | | 1.16 | 1.23 | 1.20 | 0.95 | 1.21 | 1.11 | 1.16 | 1.20 | |
| | | Average | 1.22 | 1.32 | 1.23 | 1.09 | 1.25 | 1.15 | 1.22 | 1.20 | |
| | | % RSD | 6.2 | 9.7 | 3.1 | 8.7 | 4.7 | 4.5 | 5.3 | 1.8 | |
| Appearance | Sample 1 | | Pass | Pass | Pass | Pass | Pass | Pass | Pass | Pass | Pass |
| White | Sample 2 | | Pass | Pass | Pass | Pass | Pass | Pass | Pass | Pass | |
| Foam | Sample 3 | | Pass | Pass | Pass | Pass | Pass | Pass | Pass | Pass | |
| | Sample 4 | | Pass | Pass | Pass | Pass | Pass | Pass | Pass | Pass | |

TABLE 53
Prototype A information stability results. 30 μg physical results.
1797-1111R01 - Prototype A 30 ug dose Physical Results

| | | Initial | Initial | 1 Mo @ | 1 Mo @ | 2 Mo @ | 3 Mo @ | 3 Mo @ | 6 Mo @ | 6 Mo @ | |
|---|---|---|---|---|---|---|---|---|---|---|---|
| | | (bulk) | (Foam) | 5 C. | 25 C. | 25 C. | 5 C. | 25 C. | 5 C. | 25 C. | Mean |
| | | Date | | | | | | | | | |
| | | Nov. 7, 2020 | | Dec. 17, 2020 | | Jan. 16, 2021 | Feb. 15, 2021 | | May 16, 2021 | | |
| Specific Gravity | | 0.997 | | | | | | | | | |
| pH | 6.5-7.5 | 7.1 | 7.0 | 6.9 | 7.2 | 7.3 | 7.0 | 7.1 | 7.0 | 6.8 | 7.0 |
| Foam | Sample 1 | | 23 | 18 | 29 | 23 | 24 | 29 | 27 | 25 | 26 |
| Height | Sample 2 | | 32 | 28 | 27 | 26 | 27 | 24 | 25 | 24 | |
| (mL) | Sample 3 | | 29 | 29 | 25 | 21 | 26 | 21 | 27 | 25 | RSD = 12.2% |
| | | Average | 28 | 25 | 27 | 23 | 26 | 25 | 26 | 25 | |
| | | % RSD | 16.4 | 24.3 | 7.4 | 10.8 | 6.0 | 16.4 | 4.4 | 2.3 | |
| Duration of | Sample 1 | | 1 | 1 | 0.5 | 1 | 1 | 1 | 1 | 1 | 1 |
| Expansion | Sample 2 | | 1 | 1 | 0.75 | 1 | 1 | 1 | 1 | 1 | |
| (min) | Sample 3 | | 1 | 1 | 1 | 1 | 1 | 1 | 1 | 1 | RSD = 11.6% |
| | | Average | 1 | 1 | 1 | 1 | 1 | 1 | 1 | 1 | |
| | | % RSD | 0.0 | 0.0 | 33.3 | 0.0 | 0.0 | 0.0 | 0.0 | 0.0 | |
| Weight | Sample 1 | | 1.32 | 1.18 | 1.27 | 1.21 | 1.28 | 0.98 | 1.41 | 1.14 | 1.22 |
| per | Sample 2 | | 1.30 | 1.23 | 1.24 | 2.25 | 1.31 | 1.06 | 1.21 | 1.12 | |
| Actuation | Sample 3 | | 1.27 | 1.2 | 1.25 | 1.30 | 1.30 | 1.04 | 1.22 | 1.17 | RSD = 7.6% |
| (g) | Sample 4 | | 1.24 | 1.32 | 1.23 | 1.17 | 1.19 | 1.06 | 1.28 | 1.25 | |
| | | Average | 1.28 | 1.23 | 1.25 | 1.23 | 1.27 | 1.04 | 1.28 | 1.17 | |
| | | % RSD | 2.7 | 5.0 | 1.4 | 4.5 | 4.3 | 3.7 | 7.2 | 4.9 | |
| Appearance | Sample 1 | | Pass | Pass | Pass | Pass | Pass | Pass | Pass | Pass | Pass |
| White | Sample 2 | | Pass | Pass | Pass | Pass | Pass | Pass | Pass | Pass | |
| Foam | Sample 3 | | Pass | Pass | Pass | Pass | Pass | Pass | Pass | Pass | |
| | Sample 4 | | Pass | Pass | Pass | Pass | Pass | Pass | Pass | Pass | |

TABLE 54
Prototype A information stability results. 3000 μg physical results.
1797-1111R02 - Prototype A 3000 ug dose Physical Results

| | | Initial | Initial | 1 Mo @ | 1 Mo @ | 2 Mo @ | 3 Mo @ | 3 Mo @ | 6 Mo @ | 6 Mo @ | |
|---|---|---|---|---|---|---|---|---|---|---|---|
| | | (bulk) | (Foam) | 5 C. | 25 C. | 25 C. | 5 C. | 25 C. | 5 C. | 25 C. | Mean |
| | | Date | | | | | | | | | |
| | | Nov. 7, 2020 | | Dec. 17, 2020 | | Jan. 16, 2021 | Feb. 15, 2021 | | May 16, 2021 | | |
| Specific Gravity | | 0.987 | | | | | | | | | |
| pH | 6.5-7.5 | 6.8 | 6.9 | 6.8 | 6.8 | 7.1 | 6.9 | 6.9 | 6.8 | 6.9 | 6.9 |
| Foam | Sample 1 | | 29 | 28 | 28 | 24 | 22 | 28 | 29 | 23 | 26 |
| Height | Sample 2 | | 30 | 23 | 22 | 23 | 29 | 26 | 27 | 24 | |
| (mL) | Sample 3 | | 31 | 20 | 28 | 21 | 29 | 26 | 34 | 25 | RSD = 13.5% |
| | | Average | 30 | 24 | 26 | 23 | 27 | 27 | 30 | 24 | |
| | | % RSD | 3.3 | 17.1 | 13.3 | 6.7 | 15.2 | 4.3 | 12.0 | 4.2 | |
| Duration of | Sample 1 | | 1 | 1 | 1 | 1 | 1 | 1 | 1 | 1 | 1 |
| Expansion | Sample 2 | | 1.5 | 1 | 1 | 1 | 1 | 1 | 1 | 1 | |
| (min) | Sample 3 | | 1.25 | 1 | 1 | 1 | 1 | 1 | 1 | 1 | RSD = 10.9% |
| | | Average | 1 | 1 | 1 | 1 | 1 | 1 | 1 | 1 | |
| | | % RSD | 20.0 | 0.0 | 0.0 | 0.0 | 0.0 | 0.0 | 0.0 | 0.0 | |
| Weight | Sample 1 | | 1.38 | 1.24 | 1.51 | 1.18 | 1.14 | 1.34 | 1.31 | 1.14 | 1.26 |
| per | Sample 2 | | 1.34 | 1.27 | 1.30 | 1.20 | 1.32 | 1.23 | 1.24 | 1.12 | |
| Actuation | Sample 3 | | 1.35 | 1.24 | 1.25 | 1.11 | 1.32 | 1.23 | 1.82 | 1.17 | RSD = 11.6% |
| (g) | Sample 4 | | 1.36 | 1.28 | 1.23 | 0.92 | 1.13 | 1.21 | 1.24 | 1.25 | |
| | | Average | 1.36 | 1.26 | 1.32 | 1.10 | 1.23 | 1.25 | 1.40 | 1.17 | |
| | | % RSD | 1.3 | 1.6 | 9.7 | 11.6 | 8.7 | 4.7 | 20.0 | 4.9 | |
| Appearance | Sample 1 | | Pass | Pass | Pass | Pass | Pass | Pass | Pass | Pass | Pass |
| White | Sample 2 | | Pass | Pass | Pass | Pass | Pass | Pass | Pass | Pass | |
| Foam | Sample 3 | | Pass | Pass | Pass | Pass | Pass | Pass | Pass | Pass | |
| | Sample 4 | | Pass | Pass | Pass | Pass | Pass | Pass | Pass | Pass | |

TABLE 55
Prototype A informal stability results. Analytical results: Placebo.
% Recovery in Ironwood Rectal Foam Prototype A Placebo Lot 1797-1109R02

| | | | | T1 | | T2 | T3 | | T6 | |
|---|---|---|---|---|---|---|---|---|---|---|
| | Location | Bulk | T0 | 5° C. | 25° C. | 25° C. | 5° C. | 25° C. | 5° C. | 25° C. |
| IW-3300 (API) | Beginning | | | | | | | | | |
| | Middle | | | | | | | | | |
| | End | | | | | | | | | |
| Mean | | | | | | | | | | |
| % RSD | | | | | | | | | | |
| Methylparaben | Beginning | | | 102.9 | 103.4 | 104.1 | 104.5 | 103.5 | 104.5 | 101.5 |
| | Middle | | | 101.7 | 100.4 | 103.3 | 101.2 | 101.1 | 103.8 | 102.3 |
| | End | | | 102.9 | 102.2 | 102.5 | 102.2 | 100.4 | 103.2 | 100.9 |
| Mean | | | 103.3* | 102.5 | 102.0 | 103.3 | 102.6 | 101.7 | 103.8 | 101.6 |
| % RSD | | | 0.31 | 0.68 | 1.48 | 0.79 | 1.68 | 1.57 | 0.6 | 0.71 |
| Propylparaben | Beginning | | | 102.8 | 103.9 | 105.3 | 103.7 | 103.3 | 104.4 | 102.5 |
| | Middle | | | 101.6 | 100.7 | 1042 | 100.3 | 101.2 | 103.8 | 103.'2 |
| | End | | | 103.0 | 102.8 | 103.5 | 101.5 | 100.4 | 103.1 | 101.8 |

| | | | | | | | | |
|---|---|---|---|---|---|---|---|---|
| Mean | 103.5* | 102.5 | 102.5 | 104.3 | 101.9 | 101.6 | 103.8 | 102.5 |
| % RSD | 0.30 | 0.74 | 1.59 | 0.88 | 1.67 | 1.49 | 0.60 | 0.68 |

*Mean of n = 3
Note:
Mean % Recovery at each time point is used for the graphs below

Note: The results in this Table are the average assay results and reflect the content uniformity for the can at each time point and temperature condition.

TABLE 56
Prototype A informal stability results. Analytical results: 30 μg/mL
% Recovery in Ironwood Rectal Foam Prototype A 30 μg/mL Lot 1797-1111R01

| | | | | T1 | | T2 | T3 | | T6 | |
|---|---|---|---|---|---|---|---|---|---|---|
| | Location | Bulk | T0 | 5° C. | 25° C. | 25° C. | 5° C. | 25° C. | 5° C. | 25° C. |
| IW-3300 (API) | Beginning | | | 101.0 | 97.5 | 98.6 | 102.0 | 94.4 | 101.6 | 91.0 |
| | Middle | | | 101.6 | 98.5 | 97.6 | 100.9 | 96.0 | 101.0 | 90.6 |
| | End | | | 98.5 | 93.3 | 96.9 | 101.3 | 93.3 | 101.2 | 89.7 |
| Mean | | 100.6* | 101.9* | 100.4 | 96.4 | 97.7 | 101.4 | 94.6 | 101.3 | 90.4 |
| % RSD | | 0.37 | 0.80 | 1.64 | 2.86 | 0.87 | 0.53 | 1.42 | 0.31 | 0.70 |
| Methylparaben | Beginning | | , | 101.0 | 100.0 | 99.0 | 993 | 98.0 | 100.6 | 99.1 |
| | Middle | | | 100.3 | 100.6 | 98.7 | 98.7 | 97.6 | 99.8 | 98.3 |
| | End | | | 99.4 | 99.0 | 97.1 | 98.6 | 98.5 | 99.6 | 97.2 |
| Mean | | 100.9* | 98.0** | 100.2 | 99.9 | 98.3 | 98.9 | 98.1 | 100.0 | 98.2 |
| % RSD | | 0.34 | 1.42 | 0.80 | 0.81 | 1.1 | 0.37 | 0.46 | 0.53 | 0.99 |
| Propylparaben | Beginning | | | 101.6 | 100.8 | 100.5 | 99.0 | 98.5 | 100.9 | 100.5 |
| | Middle | | | 100.8 | 101.3 | 100.5 | 98.6 | 98.1 | 100.2 | 99.9 |
| | End | | | 100.2 | 100.0 | 98.7 | 98.6 | 99.2 | 100.1 | 98.8 |

| | | | | | | | | | |
|---|---|---|---|---|---|---|---|---|---|
| Mean | 100.9* | 98.9** | 100.9 | 100.7 | 99.9 | 98.8 | 98.6 | 100.4 | 99.7 |
| % RSD | 0.39 | 1.41 | 0.70 | 0.65 | 1.1 | 0.24 | 0.54 | 0.47 | 0.87 |

*Mean of n = 3;
**Mean of n = 10
Note:
Mean % Recovery at each time point is used for the graphs below.

Note: The results in this Table are the average assay results and reflect the content uniformity for the can at each time point and temperature condition.

TABLE 57
Prototype A informal stability results. Analytical results: 3000 μg/mL.
% Recovery in Ironwood Rectal Foam Prototype A 3000 μg/mL Lot 1797-1111R02

| | | | | T1 | | T2 | T3 | | T6 | |
|---|---|---|---|---|---|---|---|---|---|---|
| | Location | Bulk | T0 | 5° C. | 25° C. | 25° C. | 5° C. | 25° C. | 5° C. | 25° C. |
| IW-3300 (API) | Beginning | | | 98.3 | 93.8 | 89.6 | 99.8 | 87.7 | 97.9 | 83.3 |
| | Middle | | | 96.6 | 93.3 | 90.2 | 97.3 | 86.5 | 96.6 | 83.4 |
| | End | | | 97.3 | 93.4 | 90.1 | 97.9 | 84.7 | 96.0 | 83.4 |
| Mean | | 92.2* | 97.5** | 97.4 | 93.5 | 90.0 | 98.3 | 843 | 96.9 | 834 |
| % RSD | | 0.41 | 1.53 | 0.88 | 0.28 | 0.37 | 1.29 | 1.76 | 1.00 | 0.06 |
| Methylparaben | Beginning | | | 100.5 | 99.0 | 97.4 | 101.6 | 98.0 | 99.1 | 94.9 |
| | Middle | | | 99.2 | 98.1 | 97.7 | 102.0 | 97.3 | 99.0 | 96.6 |
| | End | | | 98.7 | 97.7 | 98.6 | 100.5 | 96.9 | 98.3 | 95.7 |
| Mean | | 98.5* | 99.6** | 99.5 | 98.3 | 97.9 | 101.4 | 97.4 | 98.8 | 95.7 |
| % RSD | | 0.27 | 0.75 | 0.93 | 0.68 | 0.65 | 0.75 | 0.58 | 0.45 | 0.87 |
| Propylparaben | Beginning | | | 101.1 | 99.5 | 98.9 | 101.4 | 98.2 | 99.3 | 97.7 |
| | Middle | | | 99.6 | 98.6 | 99.1 | 101.8 | 97.4 | 99.2 | 99.6 |
| | End | | | 99.2 | 98.5 | 100.1 | 100.5 | 97.3 | 98.6 | 98.7 |

| | | | | | | | | | |
|---|---|---|---|---|---|---|---|---|---|
| Mean | 98.5* | 100.4** | 100.0 | 98.9 | 99.4 | 101.2 | 97.6 | 99.0 | 98.7 |
| % RSD | 0.25 | 0.79 | 1.00 | 0.56 | 0.63 | 0.46 | 0.54 | 0.38 | 0.94 |

*Mean of n = 6;
**Mean of n = 10
Note:
Mean % Recovery at each time points is used for the graphs below.
Note:
The results in this Table are the average assay results and reflect the content uniformity for the can at each time point and temperature condition.

TABLE 58
Prototype B information stability results. Placebo physical results.
1797-1109R03 - Prototype B Placebo Physical Results

| | | Initial | Initial | 1 Mo @ | 1 Mo @ | 2 Mo @ | 3 Mo @ | 3 Mo @ | 6 Mo @ | 6 Mo @ | |
|---|---|---|---|---|---|---|---|---|---|---|---|
| | | (bulk) | (Foam) | 5 C. | 25 C. | 25 C. | 5 C. | 25 C. | 5 C. | 25 C. | Mean |
| | | Date | | | | | | | | | |
| | | Nov. 7, 2020 | | Dec. 17, 2020 | | Jan. 16, 2021 | Feb. 15, 2021 | | May 16, 2021 | | |
| Specific Gravity | | 1.015 | | | | | | | | | |
| pH | 6.5-7.5 | 7.1 | 7.1 | 7.1 | 6.7 | 7.3 | 7.1 | 6.9 | 6.9 | 6.8 | 7.0 |
| Foam | Sample 1 | | 16 | 15 | 13 | 7 | 11 | 13 | 12 | 14 | 15 |
| Height | Sample 2 | | 14 | 5 | 13 | 21 | 16 | 15 | 20 | 15 | |
| (mL) | Sample 3 | | 17 | 17 | 13 | 21 | 15 | 14 | 19 | 14 | RSD = 25.9% |
| | | Average | 16 | 12 | 13 | 16 | 14 | 14 | 17 | 14 | |
| | | % RSD | 9.8 | 52.1 | 0.0 | 49.5 | 18.9 | 7.1 | 25.6 | 4.0 | |
| Duration of | Sample 1 | | 0.5 | 0.25 | 0.25 | 0.5 | 0.25 | 0.25 | 0.25 | 0.25 | 0.31 |
| Expansion | Sample 2 | | 0.5 | 0.25 | 0.25 | 0.5 | 0.25 | 0.25 | 0.25 | 0.25 | |
| (min) | Sample 3 | | 0.5 | 0.25 | 0.25 | 0.5 | 0.25 | 0.25 | 0.25 | 0.25 | RSD = 35.4% |
| | | Average | 1 | 0.3 | 0.3 | 1 | 0.3 | 0.3 | 0.3 | 0.3 | |
| | | % RSD | 0.0 | 0.0 | 0.0 | 0.0 | 0.0 | 0.0 | 0.0 | 0.0 | |
| Weight | Sample 1 | | 1.33 | 1.14 | 1.25 | 1.18 | 1.19 | 1.27 | 1.24 | 1.20 | 1.27 |
| per | Sample 2 | | 1.27 | 1.30 | 1.25 | 1.08 | 1.34 | 1.25 | 1.32 | 1.63 | |
| Actuation | Sample 3 | | 1.34 | 1.33 | 1.25 | 1.04 | 1.32 | 1.23 | 1.30 | 1.17 | RSD = 18.4% |
| (g) | Sample 4 | | 1.24 | 1.35 | 1.10 | 0.98 | 1.31 | 1.16 | 1.29 | 0.89 | |
| | | Average | 1.30 | 1.53 | 1.22 | 1.07 | 1.29 | 1.23 | 1.29 | 1.22 | |
| | | % RSD | 3.7 | 35.3 | 6.3 | 7.9 | 5.3 | 3.9 | 2.6 | 25.0 | |
| Appearance | Sample 1 | | Pass | Pass | Pass | Pass | Pass | Pass | Pass | Pass | Pass |
| White | Sample 2 | | Pass | Pass | Pass | Pass | Pass | Pass | Pass | Pass | |
| Foam | Sample 3 | | Pass | Pass | Pass | Pass | Pass | Pass | Pass | Pass | |
| | Sample 4 | | Pass | Pass | Pass | Pass | Pass | Pass | Pass | Pass | |

TABLE 59
Prototype B information stability results. 30 μg physical results.
1797-1110R01 - Prototype B 30 ug dose Physical Results

| | | Initial | Initial | 1 Mo @ | 1 Mo @ | 2 Mo @ | 3 Mo @ | 3 Mo @ | 6 Mo @ | 6 Mo @ | |
|---|---|---|---|---|---|---|---|---|---|---|---|
| | | (bulk) | (Foam) | 5 C. | 25 C. | 25 C. | 5 C. | 25 C. | 5 C. | 25 C. | Mean |
| | | Date | | | | | | | | | |
| | | Nov. 7, 2020 | | Dec. 17, 2020 | | Jan. 16, 2021 | Feb. 15, 2021 | | May 16, 2021 | | |
| Specific Gravity | | 1.005 | | | | | | | | | |
| pH | 6.5-7.5 | 7.1 | 7.1 | 7.1 | 7.1 | 7.3 | 7.0 | 7.0 | 6.9 | 6.8 | 7.0 |
| Foam | Sample 1 | | 10 | 12 | 11 | 8 | 12 | 12 | 18 | 24 | 13 |
| Height | Sample 2 | | 11 | 12 | 11 | 14 | 14 | 15 | 18 | 15 | |
| (mL) | Sample 3 | | 13 | 5 | 10 | 5 | 15 | 16 | 18 | | RSD = 33.0% |
| | | Average | 11 | 10 | 11 | 9 | 14 | 14 | 18 | 20 | |
| | | % RSD | 13.5 | 41.8 | 5.4 | 50.9 | 11.2 | 14.5 | 0.0 | 32.6 | |
| Duration of | Sample 1 | | 0.5 | 0.25 | 0.25 | 0.5 | 0.25 | 0.25 | 0.25 | 0.25 | 0.32 |
| Expansion | Sample 2 | | 0.5 | 0.25 | 0.25 | 0.5 | 0.25 | 0.25 | 0.25 | 0.25 | |
| (min) | Sample 3 | | 0.5 | 0.25 | 0.25 | 0.5 | 0.25 | 0.25 | 0.25 | | RSD = 35.6% |
| | | Average | 1 | 0.3 | 0.3 | 1 | 0.3 | 0.3 | 0.3 | 0.3 | |
| | | % RSD | 0.0 | 0.0 | 0.0 | 0.0 | 0.0 | 0.0 | 0.0 | 0.0 | |
| Weight | Sample 1 | | 1.27 | 1.22 | 1.21 | 1.05 | 1.14 | 1.11 | 1.26 | 1.85 | 1.22 |
| per | Sample 2 | | 1.29 | 1.20 | 1.33 | 1.11 | 1.30 | 1.21 | 1.26 | 0.96 | |
| Actuation | Sample 3 | | 1.36 | 1.30 | 1.23 | 0.99 | 1.27 | 1.24 | 1.29 | | |
| (g) | Sample 4 | | 1.28 | 1.35 | 1.19 | 0.96 | 1.30 | 1.16 | 0.93 | | RSD = 13.8% |
| | | Average | 1.30 | 1.27 | 1.24 | 1.03 | 1.25 | 1.18 | 1.19 | 1.41 | |
| | | % RSD | 3.1 | 5.5 | 5.0 | 6.5 | 6.1 | 4.8 | 14.4 | 44.8 | |
| Appearance | Sample 1 | | Pass | Pass | Pass | Pass | Pass | Pass | Pass | Pass | Pass |
| White | Sample 2 | | Pass | Pass | Pass | Pass | Pass | Pass | Pass | Pass | |
| Foam | Sample 3 | | Pass | Pass | Pass | Pass | Pass | Pass | Pass | | |
| | Sample 4 | | Pass | Pass | Pass | Pass | Pass | Pass | Pass | | |

Note: for Lot 1797-1110R01—Prototype B 30 μg, the missing data at 6 months at 25° C. was due to a single can being shared between stability time points for physical testing and the product in the can had been consumed.

TABLE 60
Prototype B informal stability results. 3000 μg physical results.
1797-1110R02 - Prototype B 3000 ug dose Physical Results

| | | Initial | Initial | 1 Mo @ | 1 Mo @ | 2 Mo @ | 3 Mo @ | 3 Mo @ | 6 Mo @ | 6 Mo @ | |
|---|---|---|---|---|---|---|---|---|---|---|---|
| | | (bulk) | (Foam) | 5 C. | 25 C. | 25 C. | 5 C. | 25 C. | 5 C. | 25 C. | Mean |
| | | Date | | | | | | | | | |
| | | Nov. 7, 2020 | | Dec. 17, 2020 | | Jan. 16, 2021 | Feb. 15, 2021 | | May 16, 2021 | | |
| Specific Gravity | | 1.01 | | | | | | | | | |
| pH | 6.5-7.5 | 6.9 | 7.0 | 7.0 | 6.9 | 7.1 | 6.8 | 6.9 | 6.8 | 6.7 | 6.9 |
| Foam | Sample 1 | | 15 | 8 | 18 | 20 | 11 | 15 | 16 | 14 | 15 |
| Height | Sample 2 | | 16 | 16 | 17 | 10 | 14 | 19 | 19 | 15 | |
| (mL) | Sample 3 | | 16 | 15 | 17 | 14 | 15 | 19 | 18 | 13 | RSD = 18.9% |
| | | Average | 16 | 13 | 17 | 15 | 13 | 18 | 18 | 14 | |
| | | % RSD | 3.7 | 33.5 | 3.3 | 34.3 | 15.6 | 13.1 | 8.6 | 7.1 | |
| Duration of | Sample 1 | | 0.5 | 0.25 | 0.25 | 0.5 | 0.25 | 0.25 | 0.25 | 0.25 | 0.29 |
| Expansion | Sample 2 | | 0.25 | 0.25 | 0.25 | 0.5 | 0.25 | 0.25 | 0.25 | 0.25 | |
| (min) | Sample 3 | | 0.25 | 0.25 | 0.25 | 0.5 | 0.25 | 0.25 | 0.25 | 0.25 | RSD = 32.6% |
| | | Average | 0.3 | 0.3 | 0.3 | 1 | 0.3 | 0.3 | 0.3 | 0.3 | |
| | | % RSD | 43.3 | 0.0 | 0.0 | 0.0 | 0.0 | 0.0 | 0.0 | 0.0 | |
| Weight | Sample 1 | | 1.26 | 1.15 | 1.21 | 1.11 | 1.16 | 1.31 | 1.22 | 1.14 | 1.21 |
| per | Sample 2 | | 1.23 | 1.19 | 1.37 | 1.09 | 1.20 | 1.59 | 1.26 | 1.10 | |
| Actuation | Sample 3 | | 1.23 | | 1.12 | 1.01 | 1.19 | 1.33 | 1.17 | 0.94 | RSD = 21.0% |
| (g) | Sample 4 | | 1.18 | 1.31 | 1.05 | 1.02 | 1.23 | 1.20 | 0.85 | 1.01 | |
| | | Average | 1.23 | 1.22 | 1.44 | 1.06 | 1.20 | 1.35 | 1.13 | 1.05 | |
| | | % RSD | 2.7 | 6.8 | 43.5 | 4.7 | 2.4 | 12.2 | 16.6 | 8.6 | |
| Appearance | Sample 1 | | Pass | Pass | Pass | Pass | Pass | Pass | Pass | Pass | Pass |
| White | Sample 2 | | Pass | Pass | Pass | Pass | Pass | Pass | Pass | Pass | |
| Foam | Sample 3 | | Pass | Pass | Pass | Pass | Pass | Pass | Pass | Pass | |
| | Sample 4 | | Pass | Pass | Pass | Pass | Pass | Pass | Pass | Pass | |

Note: for Lot 1797-1110R02-Prototype B 3000 μg, the weight per actuation for sample 3 at 1 month at 5° C. was inadvertently missed.

TABLE 61
Prototype B informal stability results. Analytical results: Placebo.
% Recovery in Ironwood Foam Prototype B Placebo Lot 1797-1109R03

| | | | | T1 | | T2 | T3 | | T6 | |
|---|---|---|---|---|---|---|---|---|---|---|
| | Location | Bulk | T0 | 5° C. | 25° C. | 25° C. | 5° C. | 25° C. | 5° C. | 25° C. |
| IW-3300 (API) | Beginning | | | | | | | | | |
| | Middle | | | | | | | | | |
| | End | | | | | | | | | |
| Mean | | | | | | | | | | |
| % RSD | | | | | | | | | | |
| Methylparaben | Beginning | | | 96.5 | 103.6 | 98.6 | 98.5 | 97.9 | 98.9 | 96.3 |
| | Middle | | | 99.6 | 98.6 | 99.3 | 98.5 | 98.8 | 97.9 | 95.2 |
| | End | | | 104.8 | 98.9 | 97.5 | 98.9 | 98.4 | 100.2 | 96.5 |
| Mean | | | 98.8* | 100.3 | 100.4 | 98.4 | 98.6 | 98.4 | 99.0 | 96.0 |
| % RSD | | | 0.54 | 4.18 | 2.79 | 0.94 | 0.22 | 0.46 | 1.12 | 0.72 |
| Propylparaben | Beginning | | | 95.4 | 102.7 | 98.7 | 97.0 | 98.6 | 99.4 | 97.1 |
| | Middle | | | 98.7 | 98.0 | 99.3 | 97.0 | 99.1 | 96.3 | 96.0 |
| | End | | | 103.4 | 98.7 | 98.2 | 97.5 | 98.9 | 97.6 | 97.7 |

| | | | | | | | | |
|---|---|---|---|---|---|---|---|---|
| Mean | 98.5* | 99.2 | 99.8 | 98.7 | 97.2 | 98.9 | 97.7 | 963 |
| % RSD | 0.54 | 4.05 | 2.54 | 0.57 | 0.27 | 0.29 | 1.58 | 0.92 |

*Mean of n = 3
Note:
Mean % Recovery at each time points is used for the graphs below.
Note:
The results in this Table are the average assay results and reflect the content uniformity for the can at each time point and temperature condition.

TABLE 62
Prototype B informal stability results. Analytical results: 30 μg/mL
% Recovery in Ironwood Rectal Foam Prototype B 30 μg/mL Lot 1797-1110801

| | | | | T1 | | T2 | T3 | | T6 | |
|---|---|---|---|---|---|---|---|---|---|---|
| | Location | Bulk | T0 | 5° C. | 25° C. | 25° C. | 5° C. | 25° C. | 5° C. | 25° C. |
| IW-3300 (API) | Beginning | | | | 97.3 | 97.3 | 102.1 | 95.3 | 101.8 | 90.6 |
| | Middle | | | 99.5 | 96.2 | 98.9 | 1023 | 96.3 | 101.8 | 91.2 |
| | End | | | 99.6 | 964 | 98.2 | 101.9 | 95.2 | 101.5 | 91.5 |
| Mean | | 99.6* | 100.1** | 99.8 | 96.6 | 98.1 | 102.1 | 95.6 | 101.7 | 91.1 |
| % RSD | | 0.5 | 0.5 | 0.38 | 0.61 | 0.85 | 0.19 | 0.64 | 0.19 | 0.51 |
| Methylperaben | Beginning | | | 98.9 | 97.5 | 98.4 | 97.5 | 98.1 | 98.3 | 95.7 |
| | Middle | | | 100.4 | 98.2 | 98.5 | 98.8 | 97.8 | 97.1 | 96.1 |
| | End | | | 99.2 | 98.7 | 96.9 | 98.1 | 96.8 | 96.8 | 96.6 |
| Mean | | 101.1* | 99.8.** | 99.5 | 98.1 | 97.9 | 98.1 | 97.6 | 97.4 | 96.1 |
| % RSD | | 0.29 | 1.31 | 0.80 | 0.61 | 0.89 | 0.64 | 0.67 | 0.81 | 0.44 |
| Propylparaben | Beginning | | | 96.1 | 95.8 | 97.4 | 94.1 | 96.6 | 95.4 | 95.1 |
| | Middle | | | 97.8 | 96.1 | 97.0 | 96.0 | 95.8 | 94.3 | 95.3 |
| | End | | | 96.3 | 96.7 | 96.3 | 94.9 | 95.5 | 94.0 | 96.2 |

| | | | | | | | | | |
|---|---|---|---|---|---|---|---|---|---|
| Mean | 99.0* | 97.6** | 96.7 | 96.2 | 96.9 | 95.0 | 95.9 | 94.5 | 95.5 |
| % RSD | 0.40 | 1.17 | 0.96 | 0.48 | 0.57 | 0.97 | 0.60 | 0.78 | 0.51 |

*Mean of n = 6;
**Mean of n = 10
Note.
Mean % Recovery at each time points is used for the graphs below
Note:
The results in this Table are the average assay results and reflect the content uniformity for the can at each time point and temperature condition.

Note: The results in this Table are the average assay results and reflect the content uniformity for the can at each time point and temperature condition.

TABLE 63
Prototype B informal stability results. Analytical results: 3000 μg/mL.
% Recovery in Ironwood Rectal Foam Prototype B 3000 μg/mL Lot 1797-1110R02

| | | | | T1 | | T2 | T3 | | T6 | |
|---|---|---|---|---|---|---|---|---|---|---|
| | Location | Bulk | T0 | 5° C. | 25° C. | 25° C. | 5° C. | 25° C. | 5° C. | 25° C. |
| IW-3300 (APO) | Beginning | | | 101.7 | 99.0 | 97.5 | 102.6 | 91.4 | 102.6 | 86.1 |
| | Middle | | | 101.0 | 97.8 | 94.6 | 99.6 | 90.0 | 102.7 | 86.5 |
| | End | | | 102.1 | 98.0 | 95.6 | 100.8 | 90.0 | 102.8 | 86.8 |
| Mean | | 98.9* | 100.2** | 101.6 | 98.3 | 95.9 | 101.0 | 90.5 | 102.7 | 86.5 |
| % RSD | | 0.97 | 0.67 | 0.55 | 0.65 | 1.54 | 149 | 0.93 | 0.11 | 0.42 |
| Methylparaben | Beginning | | | 95.5 | 94.4 | 97.6 | 97.0 | 95.2 | 95.8 | 95.2 |
| | Middle | | | 97.5 | 95.6 | 95.3 | 96.3 | 95.4 | 96.5 | 94.3 |
| | End | | | 972 | 95.6 | 95.3 | 95.7 | 95.1 | 97.1 | 94.7 |
| Mean | | 98.3* | 97.7** | 96.7 | 95.2 | 96.1 | 96.4 | 95.2 | 96.4 | 94.7 |
| % RSD | | 0.32 | 0.53 | 1.12 | 0.73 | 1.39 | 0.66 | 0.17 | 0.68 | 0.09 |
| Propylparaben | Beginning | | | 97.3 | 95.9 | 100.1 | 96.9 | 96.4 | 96.6 | 97.4 |
| | Middle | | | 99.1 | 96.8 | 97.6 | 96.0 | 96.6 | 97.2 | 96.6 |
| | End | | | 98.5 | 97.0 | 97.4 | 95.9 | 96.5 | 97.9 | 96.8 |

| | | | | | | | | | |
|---|---|---|---|---|---|---|---|---|---|
| Mean | 98.6* | 99.3** | 98.3 | 96.6 | 98.4 | 96.3 | 96.5 | 97.2 | 96.9 |
| % RSD | 0.26 | 0.72 | 0.93 | 0.61 | 1.55 | 0.59 | 0.10 | 0.67 | 0.40 |

*Mean of 16;
** Mean of n = 10
Note:
Mean % Recovery at each time points is used for the graphs below
Note:
The results in this Table are the average assay results and reflect the content uniformity for the can at each time point and temperature condition.

For Prototype A, from the informal stability for Placebo, 30 μg, and 3000 μg were consistent for the physical parameters evaluated throughout the study. It was observed that Prototype A produced a stable foam upon actuation. For Prototype A, the analytical results for Active and Preservatives in the Placebo, 30 μg and 3000 μg were steady throughout the study. The only observation is that at 25° C. the API concentration dropped in the 30 μg and 3000 μg samples over the course of the study. Thus, the need to store the foam product at 5° C. until use.

For Prototype B, from the informal stability for Placebo, 30 μg and 3000 μg were reliable for the physical parameters evaluated throughout the study. The only observation, while not runny or fluid, was that the foam for Prototype B collapsed after the foam height per actuation was achieved. For Prototype B, the analytical results for Active and Preservatives in the Placebo, 30 μg and 3000 μg were steady throughout the study. In Prototype B as well, at 25° C. the API concentration dropped in the 30 μg and 3000 μg samples over the course of the study.

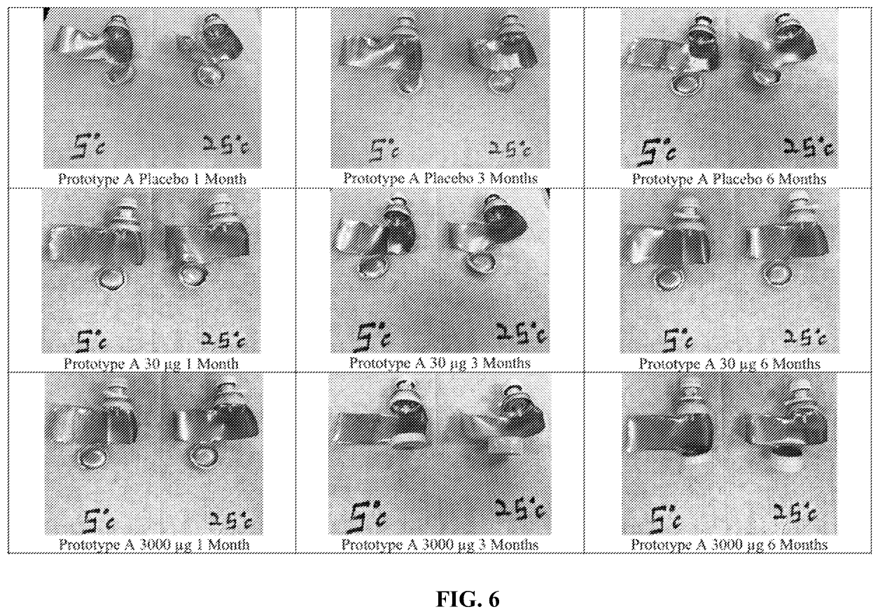

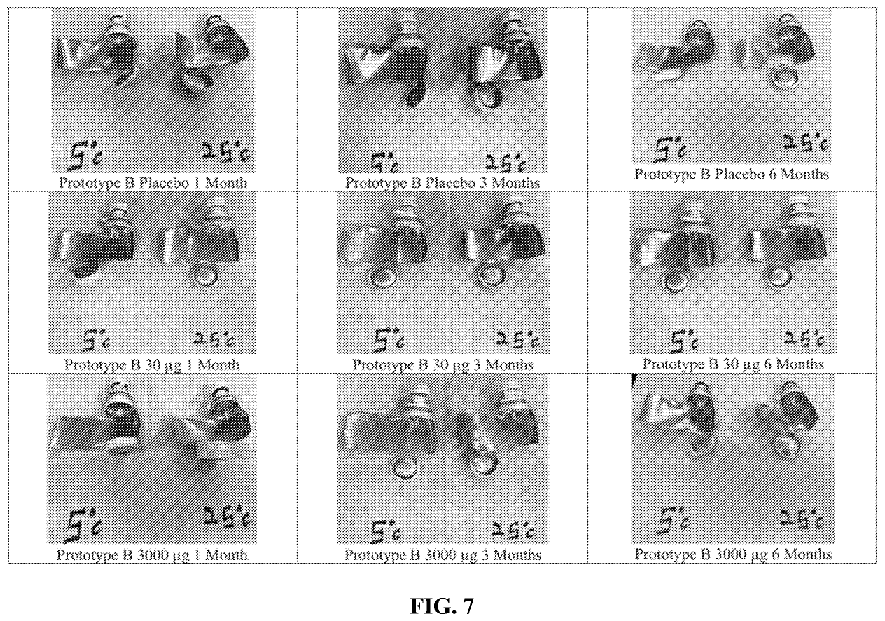

FIGS. 6-7 show that there were no visual signs of pitting or discoloration c observed on the cans for Prototypes A and B during the informal stability program.

### Example 25. Formula Enhancement

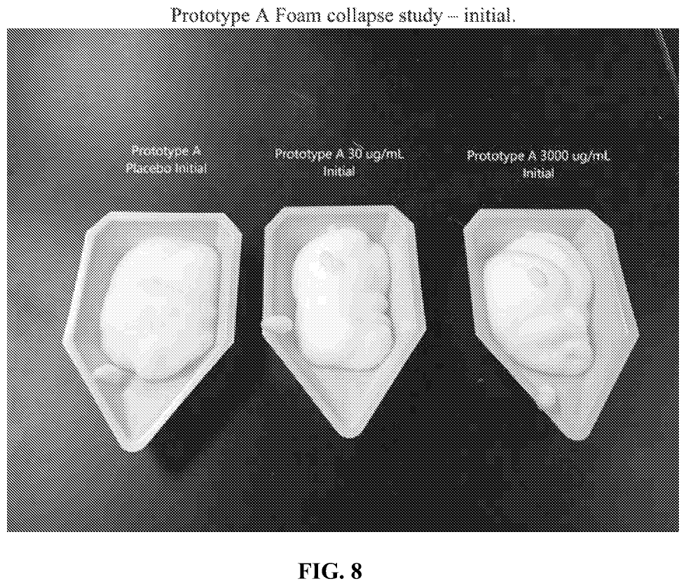

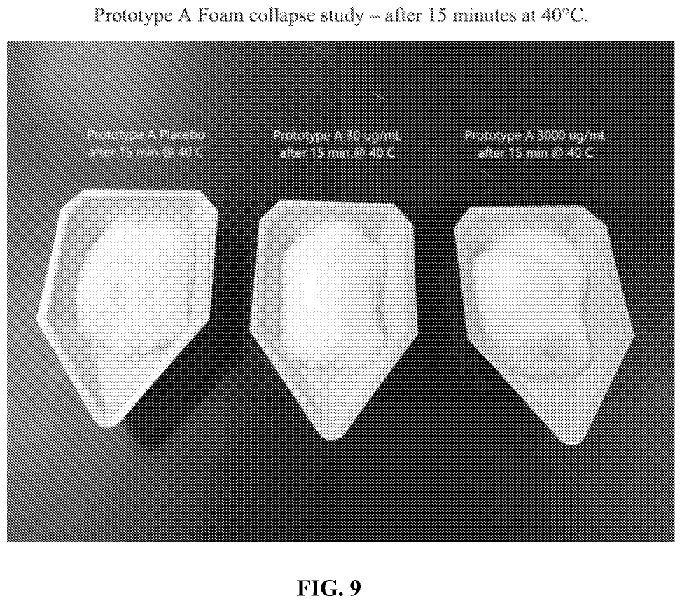

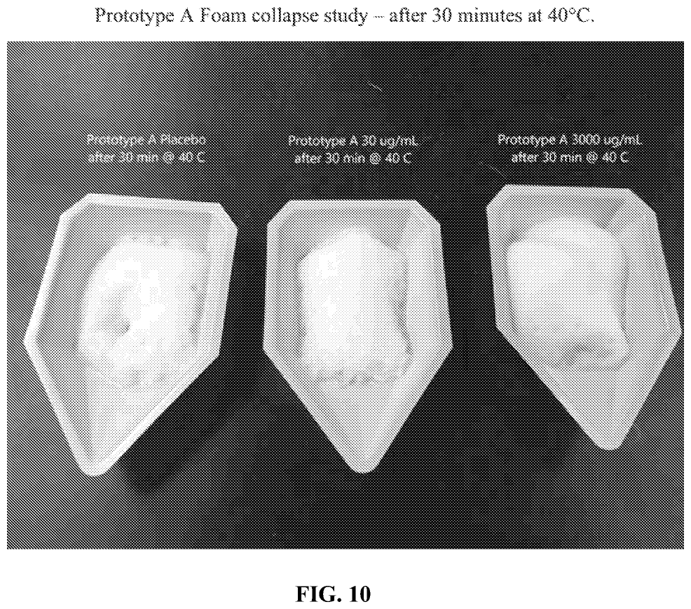

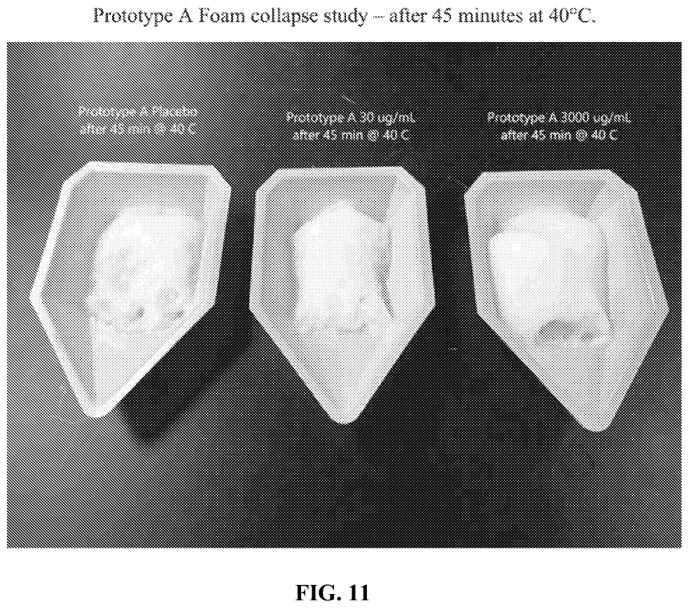

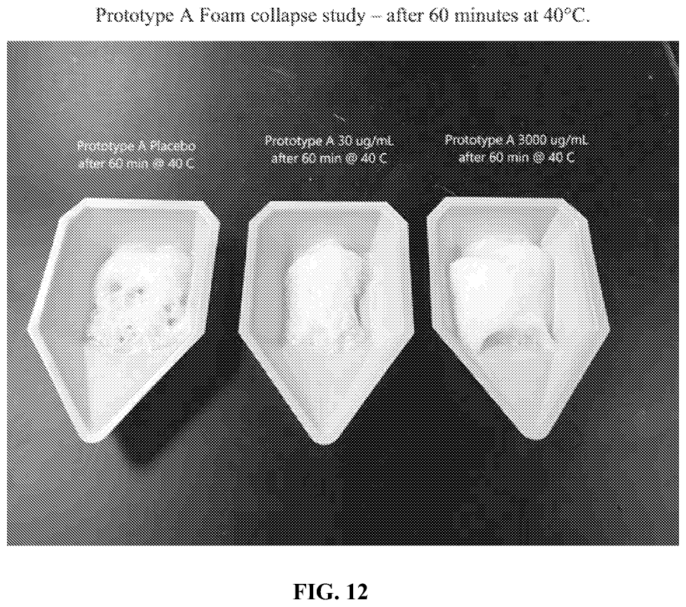

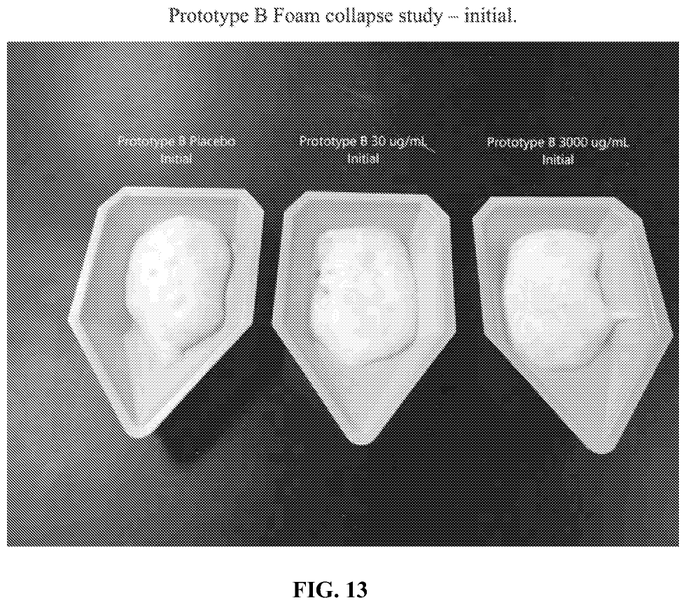

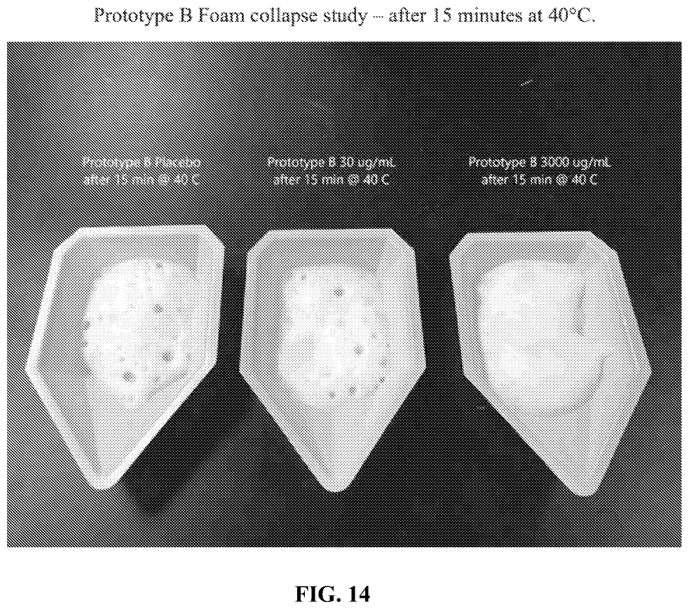

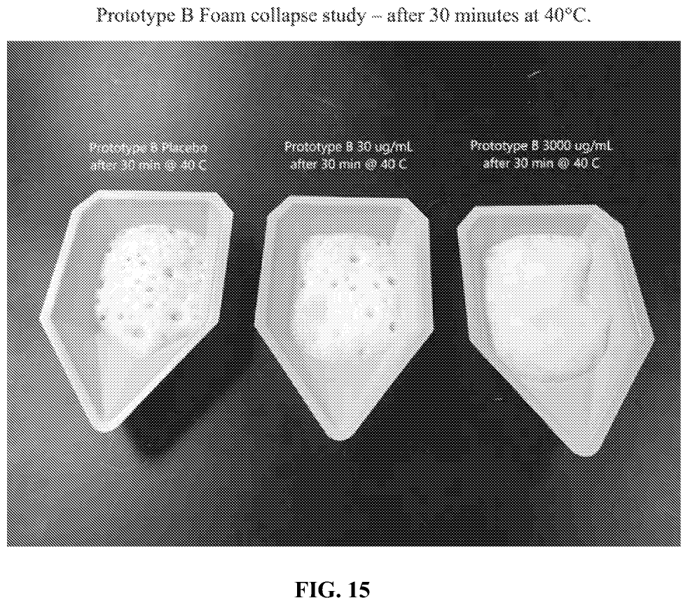

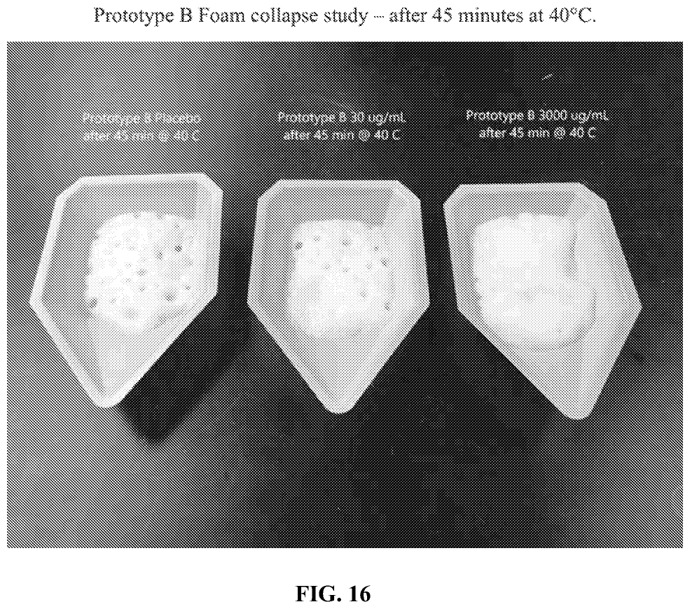

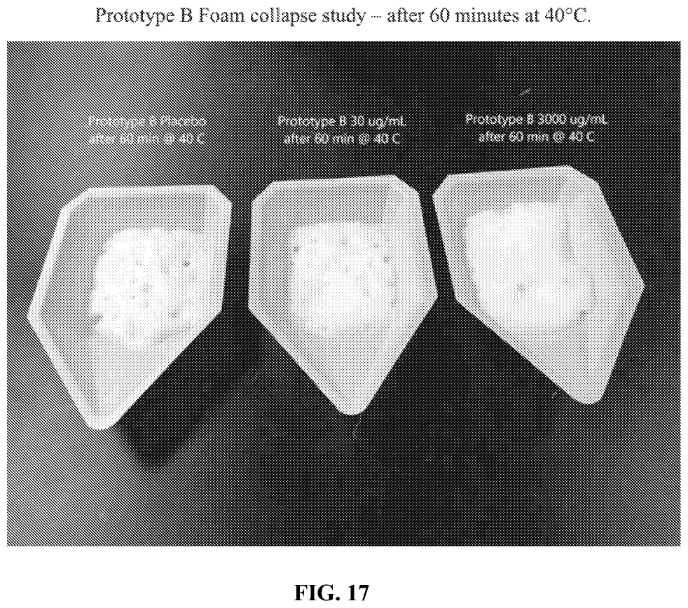

Based on the creamier foam appearance actuated without a collapse in volume and texture which was observed during the informal stability, Prototype A was selected as the pharmaceutical composition going forward. Prototype A had greater foam volume (see tables 51-54) and did not collapse as compared to prototype B. Prototype B collapsed after each actuation. Prototype A, as compared to Prototype B, would remain intact providing better drug delivery to the patient which is preferred for retaining the drug for longer time at the site of action. A foam collapse study was performed on these Prototypes to show how these foams held up. This was accomplished by actuating one actuation into a weigh boat which was then placed into a 40° C. oven. Photos were taken of these samples at initial, 15, 30 45 and 60 minutes. Prototype A produced a creamy, intact foam which remained consistent through at least 30 minutes. Prototype B appeared to collapse at the initial and was flat at the 15-minute time point. FIGS. 8-17.

One concern was the actuation amount dispensed and what would be considered an acceptable range. Based on the mean and RSD values (Tables 51-54) the amount actuated was less than the actuator target capacity (1.35 mL) and had wider variability than desired. It was determined that the product needed to be optimized using different propellants at different levels to achieve a narrow range for actuated amounts. Further enhancements to Prototype A were performed using the Placebo formulation; other propellants were re-evaluated with Prototype A. Table 62 below provides the propellants used for consideration in the pharmaceutical rectal foam composition as performed during formulation development. This table provides the propellant name, the PSI specification range for the propellant and the composition and ratios in these propellants.

TABLE 64
Propellants for enhanced pharmaceutical rectal foam composition.

| Propellant | PSI | Composition(s)/ratios |
|---|---|---|
| DME | 49-55 | Dimethyl Ether/n-Butane (53/47) |
| A17 | 15-19 | n-Butane (>97) |
| A31 | 29-33 | Isobutane (>95) |
| AP35 | 33-37 | Isobutane (83-89)/n-Butane(5-9)/Propane(5-9) |
| A46 | 44-48 | Isobutane (76.6-88)/Propane (12-21.9) |
| A48 | 46-50 | n-Butane (30-60)/Isobutane (15-30)/Propane (20-45) |
| A70 | 68-72 | Isobutane (46-64)/Propane (35.7-52.3)/n-Butane (0-3) |
| AP70 | 68-72 | Propane (49.8-59.8)/n-Butane (22.3-32.3)/Isobutanene(12.9-22.9) |

PSI = pounds per square inch.

Bulk product was packaged in a 30-g aluminum aerosol cans with a metered dose valve. Each can contained 31 g of bulk and gassed with approximately 3.0 g (target range: 2.8 g-3.2 g) of each propellant. For some cans the amount of propellant was lower than the targeted range due to the manual filling. Based on the data shown in tables 63-66, the propellants DME and AP35 were selected for additional studies. Ten cans of Prototype A Placebo, each with either DME or AP35, along with previously actuated cans with AP70 were compared (data not shown). Some cans were gassed with propellant levels lower or higher than the targeted range. This was done to observe the performance of the actuated amount dispensed and to define what is an acceptable propellant range.

Actuations were performed as follows: Each can was equilibrated in a 25° C. water batch for at least 30 minutes. The can was removed and initially shaken for 30 seconds prior to the first actuation. For each actuation the can was inverted, the meter head pressed for at least 5 seconds and then slowly released. This was followed with 3 more actuations. The can was placed back into the 25° C. water bath for at least 5 minutes before the next set of 4 actuations were performed.

The results are shown in tables 63-66 below.

TABLE 65
Propellant evaluation. Here, Prototype A Placebo was used.

| | DME - 31 g/2.8 g | | | DME 31 g/3.4 g | | | DME 31 g/2.8 g | | |
|---|---|---|---|---|---|---|---|---|---|
| | | Can | | | Can | | | Can | |
| | Can | weight | Foam | Can | weight | Foam | Can | weight | Foam |
| Actuation | weight | difference | Height | weight | difference | Height | weight | difference | Height |
| # | (g) | (g) | (mL) | (g) | (g) | (mL) | (g) | (g) | (mL) |
| 0 | 54.42 | | | 55.07 | | | 54.42 | | |
| 1 | 53.22 | 1.20 | 26 | 53.83 | 1.24 | 35 | 53.16 | [symbol] | 31 |
| 2 | 51.93 | 1.29 | | 52.56 | 1.27 | | 51.96 | 1.20 | |
| 3 | 50.65 | 1.28 | | 51.36 | 1.20 | | 50.83 | 1.13 | |
| 4 | 49.47 | 1.18 | | 50.23 | 1.13 | | 49.72 | 1.11 | |
| 5 | 48.30 | 1.17 | 27 | 49.14 | 1.09 | 31 | 48.64 | 1.08 | 25 |
| 6 | 47.16 | 1.14 | | 48.04 | 1.10 | | 47.55 | 1.09 | |
| 7 | 46.11 | 1.05 | | [symbol] | 1.08 | | 46.51 | 1.04 | |
| 8 | 45.06 | 1.05 | | 45.90 | 1.06 | | 45.45 | 1.06 | |
| 9 | 44.02 | 1.04 | 25 | 44.81 | 1.09 | 29 | 44.39 | [symbol] | 23 |
| 10 | 42.96 | 1.06 | | 43.70 | 1.11 | | 43.34 | 1.05 | |
| 11 | 41.90 | 1.06 | | 42.58 | 1.12 | | 42.28 | [symbol] | |
| 12 | 40.87 | 1.03 | | 41.46 | 1.12 | | 41.23 | 1.05 | |
| 13 | 39.74 | 1.13 | 27 | 40.33 | 1.13 | 30 | 40.14 | 1.09 | 23 |
| 14 | 38.66 | 1.08 | | 39.19 | 1.14 | | 39.03 | 1.11 | |
| 15 | 37.57 | 1.09 | | [symbol] | 1.11 | | [symbol] | 1.11 | |
| 16 | 36.49 | 1.08 | | 36.91 | 1.17 | | [symbol] | 1.13 | |
| 17 | 35.44 | 1.05 | 24 | 35.71 | 1.20 | 32 | 35.68 | 1.11 | 26 |
| 18 | 34.33 | 1.11 | | 34.53 | 1.18 | | 34.52 | 1.16 | |
| 19 | 33.23 | 1.10 | | [symbol] | 1.20 | | 33.37 | 1.15 | |
| 20 | 32.13 | 1.10 | | 32.10 | 1.23 | | 32.20 | 1.17 | |
| 21 | 30.92 | 1.21 | 26 | 30.89 | 1.21 | 32 | 31.00 | 1.20 | 24 |
| 22 | | | | | | | | | |
| | Mean | 1.12 | | Mean | 1.15 | | Mean | 1.12 | |
| | % RSD | 6.8 | | % RSD | 5.1 | | % RSD | 5.2 | |
| | Total (g) | 23.50 | | Total (g) | 24.18 | | Total (g) | 23.42 | |
| | for 21 actuations | | | | | | | | |
| | | Overall (10) | 1.12 | | | | | | |
| | | % RSD (10) | 8.2 | | | | | | |

| | DME 31 g/2.9 g | | | DME 31 g/2.4 g | | |
|---|---|---|---|---|---|---|
| | | Can | | | Can | |
| | Can | weight | Foam | Can | weight | Foam |
| Actuation | weight | difference | Height | weight | difference | Height |
| # | (g) | (g) | (mL) | (g) | (g) | (mL) |
| 0 | 54.72 | | | 54.10 | | |
| 1 | 53.49 | 1.23 | 22 | 52.75 | 1.35 | 25 |
| 2 | 52.19 | 1.30 | | 51.41 | 1.34 | |
| 3 | 50.88 | 1.31 | | 50.14 | 1.27 | |
| 4 | [symbol] | 1.24 | | 48.94 | 1.20 | |
| 5 | 48.44 | 1.20 | 31 | 47.77 | 1.17 | 25 |
| 6 | 47.34 | 1.10 | | 46.68 | 1.09 | |
| 7 | 46.32 | 1.02 | | [symbol] | 1.05 | |
| 8 | 45.30 | 1.02 | | 44.62 | 1.01 | |
| 9 | 44.20 | 1.10 | 29 | 43.64 | 0.98 | 20 |
| 10 | 43.19 | 1.01 | | [symbol] | 0.95 | |
| 11 | 42.18 | 1.01 | | 41.72 | 0.97 | |
| 12 | 41.20 | 0.98 | | 40.81 | 0.91 | |
| 13 | 40.04 | 1.16 | 29 | 39.87 | 0.94 | 19 |
| 14 | [symbol] | 1.04 | | [symbol] | 0.94 | |
| 15 | 37.96 | 1.04 | | 37.98 | 0.95 | |
| 16 | 36.87 | 1.09 | | 37.03 | 0.95 | |
| 17 | 35.76 | 1.11 | 27 | 36.05 | 0.98 | 19 |
| 18 | 34.67 | 1.09 | | 34.82 | 1.23 | |
| 19 | 33.55 | 1.12 | | 33.79 | 1.03 | |
| 20 | 32.41 | 1.14 | | 32.74 | 1.05 | |
| 21 | 31.24 | 1.17 | 27 | 31.59 | 1.15 | 21 |
| 22 | | | | | | |
| | Mean | 1.12 | | Mean | 1.07 | |
| | % RSD | 8.6 | | % RSD | 12.9 | |
| | Total (g) | 23.48 | | Total (g) | 22.51 | |

[symbol]

indicates data missing or illegible when filed

TABLE 66
Propellant evaluation. Here, Prototype A Placebo was used.

| | DME - 31 g/2.9 g | | | DME 31 g/3.15 g | | | DME 31 g/3.1 g | | |
|---|---|---|---|---|---|---|---|---|---|
| | | Can | | | Can | | | Can | |
| | Can | weight | Foam | Can | weight | Foam | Can | weight | Foam |
| Actuation | weight | difference | Height | weight | difference | Height | weight | difference | Height |
| # | (g) | (g) | (mL) | (g) | (g) | (mL) | (g) | (g) | (mL) |
| 0 | 54.85 | | | 54.88 | | | 54.57 | | |
| 1 | [symbol] | 1.47 | 25 | 53.73 | 1.15 | 26 | 53.42 | 1.15 | 31 |
| 2 | 52.08 | 1.30 | | 52.5 | 1.23 | | 52.16 | 1.26 | |
| 3 | 50.80 | 1.28 | | 51.24 | 1.26 | | 50.94 | 1.22 | |
| 4 | 49.57 | 1.23 | | 50.03 | 1.21 | | 49.78 | [symbol] | |
| 5 | 48.38 | 1.19 | 32 | 48.95 | 1.08 | 32 | 48.67 | 1.11 | 33 |
| 6 | 47.29 | 1.09 | | 47.88 | 1.07 | | [symbol] | 1.06 | |
| 7 | 46.22 | 1.07 | | 46.86 | 1.02 | | 46.56 | 1.05 | |
| 8 | 45.21 | 1.01 | | 45.85 | 1.01 | | [symbol] | 1.00 | |
| 9 | 44.15 | 1.06 | 27 | [symbol] | 0.99 | 28 | 44.51 | 1.05 | 29 |
| 10 | 43.12 | 1.03 | | 43.86 | 1.00 | | 43.45 | 1.06 | |
| 11 | 42.09 | 1.03 | | 42.87 | 0.99 | | 42.42 | 1.03 | |
| 12 | 41.07 | 1.02 | | 41.87 | 1.00 | | 41.37 | 1.05 | |
| 13 | 39.87 | 1.20 | 31 | 40.84 | 1.03 | 28 | 40.29 | 1.08 | [symbol] |
| 14 | 38.83 | 1.04 | | 39.78 | 1.06 | | 39.20 | 1.09 | |
| 15 | 37.71 | 1.12 | | 38.72 | 1.06 | | 38.07 | 1.13 | |
| 16 | 36.60 | 1.11 | | [symbol] | [symbol] | | 36.88 | 1.19 | |
| 17 | 35.47 | 1.13 | 31 | 36.53 | 1.13 | 31 | 35.71 | 1.17 | 31 |
| 18 | 34.34 | 1.13 | | 35.38 | 1.15 | | 34.52 | 1.19 | |
| 19 | 33.18 | 1.16 | | 34.26 | 1.12 | | 33.29 | [symbol] | |
| 20 | 31.96 | 1.22 | | 33.13 | 1.13 | | 32.09 | 1.20 | |
| 21 | 30.76 | 1.20 | 28 | 31.92 | [symbol] | 31 | 30.84 | 1.25 | 31 |
| 22 | | | | | | | | | |
| | Mean | 1.15 | | Mean | 1.09 | | Mean | 1.13 | |
| | % RSD | 9.9 | | % RSD | 7.7 | | % RSD | 7.0 | |
| | Total (g) | 24.09 | | Total (g) | 22.96 | | Total (g) | 23.73 | |
| | for 21 actuations | | | | | | | | |

| | DME 31 g/3.2 g | | | DME 31 g/2.8 g | | |
|---|---|---|---|---|---|---|
| | | Can | | | Can | |
| | Can | weight | Foam | Can | weight | Foam |
| Actuation | weight | difference | Height | weight | difference | Height |
| # | (g) | (g) | (mL) | (g) | (g) | (mL) |
| 0 | 54.93 | | | 54.54 | | |
| 1 | 53.71 | 1.22 | 25 | 53.28 | 1.26 | 28 |
| 2 | 52.45 | 1.26 | | 52.01 | 1.27 | |
| 3 | 51.20 | 1.25 | | 50.78 | [symbol] | |
| 4 | 50.02 | 1.18 | | 49.56 | 1.22 | |
| 5 | 48.90 | 1.12 | 35 | 48.47 | 1.09 | 28 |
| 6 | [symbol] | 1.06 | | 47.38 | 1.09 | |
| 7 | 46.82 | 1.02 | | [symbol] | 1.03 | |
| 8 | 45.77 | 1.05 | | 45.35 | 1.00 | |
| 9 | 44.66 | 1.11 | 33 | 44.35 | 1.00 | 24 |
| 10 | 43.59 | 1.07 | | [symbol] | 0.97 | |
| 11 | 42.55 | 1.04 | | 42.36 | 1.02 | |
| 12 | 41.49 | 1.06 | | 41.34 | 1.02 | |
| 13 | 40.38 | 1.11 | 32 | [symbol] | 1.06 | 26 |
| 14 | 39.19 | 1.19 | | 39.26 | 1.02 | |
| 15 | 37.99 | 1.20 | | 38.18 | 1.08 | |
| 16 | 36.85 | 1.14 | | 37.13 | 1.05 | |
| 17 | 35.65 | 1.20 | 33 | 36.00 | [symbol] | 27 |
| 18 | 34.43 | 1.22 | | 34.83 | 1.17 | |
| 19 | 33.22 | 1.21 | | 33.66 | 1.17 | |
| 20 | 32.01 | 1.21 | | 32.43 | [symbol] | |
| 21 | 30.78 | 1.23 | 32 | 31.29 | 1.14 | 26 |
| 22 | | | | | | |
| | Mean | 1.15 | | Mean | 1.11 | |
| | % RSD | 6.7 | | % RSD | 8.6 | |
| | Total (g) | 24.15 | | Total (g) | 23.25 | |

[symbol]

indicates data missing or illegible when filed

TABLE 67
Propellant evaluation. Here, Prototype A Placebo was used.

| | AP35 - 31 g/2.8 g | | | AP35 - 31 g/3.7 g | | | AP35 - 31 g/3.3 g | | |
|---|---|---|---|---|---|---|---|---|---|
| | | Can | | | Can | | | Can | |
| | Can | weight | Foam | Can | weight | Foam | Can | weight | Foam |
| Actuation | weight | difference | Height | weight | difference | Height | weight | difference | Height |
| # | (g) | (g) | (mL) | (g) | (g) | (mL) | (g) | (g) | (mL) |
| 0 | 54.66 | | | 55.15 | | | 56.97 | | |
| 1 | 53.47 | 1.19 | 30 | [symbol] | 1.17 | 34 | 55.87 | 1.10 | 39 |
| 2 | 52.18 | 1.29 | | 52.76 | 1.22 | | 54.67 | 1.20 | |
| 3 | 50.92 | 1.26 | | 51.56 | 1.20 | | 53.49 | 1.18 | |
| 4 | 49.70 | 1.22 | | 50.39 | 1.17 | | 52.28 | 1.21 | |
| 5 | 48.54 | 1.16 | [symbol] | [symbol] | 1.11 | 32 | 51.14 | 1.14 | 29 |
| 6 | 47.43 | 1.11 | | 48.20 | 1.08 | | 50.04 | 1.10 | |
| 7 | [symbol] | 1.06 | | 47.09 | 1.11 | | [symbol] | 1.03 | |
| 8 | 45.28 | 1.09 | | 45.98 | 1.11 | | [symbol] | 1.01 | |
| 9 | 44.24 | 1.04 | 22 | 44.87 | 1.11 | 31 | 46.88 | 1.12 | 29 |
| 10 | 43.19 | 1.05 | | [symbol] | 1.15 | | 45.71 | 1.17 | |
| 11 | 42.14 | 1.05 | | 42.53 | 1.19 | | 44.63 | 1.08 | |
| 12 | 41.10 | 1.04 | | 41.39 | 1.14 | | [symbol] | 1.15 | |
| 13 | 40.04 | [symbol] | 22 | 40.24 | 1.15 | 32 | [symbol] | 1.20 | 29 |
| 14 | [symbol] | 1.06 | | 39.04 | 1.20 | | 41.09 | 1.19 | |
| 15 | 37.89 | 1.09 | | 37.82 | 1.22 | | 39.90 | 1.19 | |
| 16 | 36.74 | 1.15 | | 36.55 | 1.27 | | [symbol] | 1.25 | |
| 17 | 35.62 | 1.12 | 23 | 35.31 | 1.24 | 35 | 37.34 | 1.31 | 31 |
| 18 | 34.50 | 1.12 | | 34.08 | 1.23 | | 36.08 | 1.26 | |
| 19 | 33.33 | 1.17 | | 32.82 | 1.26 | | 34.79 | 1.29 | |
| 20 | 32.15 | 1.18 | | 31.54 | 1.28 | | 33.49 | 1.30 | |
| 21 | 30.98 | 1.17 | 26 | 30.27 | 1.27 | 34 | 32.32 | 1.17 | 30 |
| 22 | | | | | | | | | |
| | Mean | 1.13 | | Mean | 1.18 | | Mean | 1.17 | |
| | % RSD | 6.5 | | % RSD | 5.2 | | % RSD | 7.0 | |
| | Total (g) | 23.68 | | Total (g) | 24.88 | | Total (g) | 24.65 | |
| | for 21 actuations | | | | | | | | |
| | | Overall (10) | 1.16 | | | | | | |
| | | STDEV | 0.09 | | | | | | |
| | | % RSD (10) | 7.7 | | | | | | |

| | AP35 - 31 g/3.3 g | | | AP35 - 31 g/3.2 g | | |
|---|---|---|---|---|---|---|
| | | Can | | | Can | |
| | Can | weight | Foam | Can | weight | Foam |
| Actuation | weight | difference | Height | weight | difference | Height |
| # | (g) | (g) | (mL) | (g) | (g) | (mL) |
| 0 | 55.06 | | | 55.49 | | |
| 1 | 53.76 | 1.30 | 34 | 54.31 | 1.18 | 24 |
| 2 | 52.47 | 1.29 | | 53.01 | 1.30 | |
| 3 | 51.09 | 1.38 | | 51.79 | 1.22 | |
| 4 | 49.93 | 1.16 | | 50.61 | 1.18 | |
| 5 | 48.81 | 1.12 | 37 | 49.54 | 1.07 | 30 |
| 6 | 47.68 | 1.13 | | 48.48 | 1.06 | |
| 7 | 46.63 | 1.05 | | 47.43 | 1.05 | |
| 8 | 45.60 | 1.03 | | 46.41 | 1.02 | |
| 9 | 44.50 | 1.10 | 29 | 45.27 | 1.14 | 30 |
| 10 | [symbol] | 1.11 | | 44.22 | 1.05 | |
| 11 | 42.28 | 1.11 | | 43.16 | 1.06 | |
| 12 | 41.11 | 1.17 | | 42.09 | 1.07 | |
| 13 | 39.90 | 1.21 | 31 | 40.95 | 1.14 | 30 |
| 14 | [symbol] | 1.28 | | 39.82 | 1.13 | |
| 15 | 37.38 | 1.24 | | 38.62 | 1.20 | |
| 16 | 36.10 | 1.28 | | 37.42 | [symbol] | |
| 17 | 34.82 | 1.28 | 35 | 36.20 | 1.22 | 32 |
| 18 | 33.55 | 1.27 | | 34.98 | 1.22 | |
| 19 | 32.27 | 1.28 | | 33.74 | 1.24 | |
| 20 | 31.01 | 1.26 | | 32.51 | 1.23 | |
| 21 | 29.74 | 1.27 | 32 | 31.24 | 1.27 | 31 |
| 22 | | | | | | |
| | Mean | 1.21 | | Mean | 1.15 | |
| | % RSD | 7.9 | | % RSD | 7.2 | |
| | Total (g) | 25.32 | | Total (g) | 24.25 | |

[symbol]

indicates data missing or illegible when filed

TABLE 68
Propellant evaluation. Here, Prototype A Placebo was used.

| | AP35 - 31 g/3.0 g | | | AP35 - 31 g/3.3 g | | | AP35 - 31 g/3.2 g | | |
|---|---|---|---|---|---|---|---|---|---|
| | | Can | | | Can | | | Can | |
| | Can | weight | Foam | Can | weight | Foam | Can | weight | Foam |
| Actuation | weight | difference | Height | weight | difference | Height | weight | difference | Height |
| # | (g) | (g) | (mL) | (g) | (g) | (mL) | (g) | (g) | (mL) |
| 0 | 54.48 | | | 55.05 | | | 54.98 | | |
| 1 | [symbol] | 1.20 | 32 | 53.94 | 1.11 | 25 | 53.78 | 1.20 | 30 |
| 2 | 52.01 | 1.27 | | 52.62 | 1.32 | | [symbol] | 1.26 | |
| 3 | 50.75 | 1.26 | | 51.36 | 1.26 | | 51.30 | 1.22 | |
| 4 | 49.58 | 1.17 | | 50.15 | 1.21 | | 50.14 | 1.16 | |
| 5 | 48.49 | 1.09 | 28 | 49.10 | 1.05 | 31 | 48.95 | 1.19 | 33 |
| 6 | 47.44 | 1.05 | | 47.96 | 1.14 | | 47.89 | 1.05 | |
| 7 | 46.42 | 1.02 | | 46.91 | 1.05 | | 46.87 | [symbol] | |
| 8 | 45.42 | 1.00 | | 45.92 | 0.99 | | 45.88 | 0.99 | |
| 9 | 44.36 | 1.05 | 25 | 44.73 | 1.19 | 33 | 44.68 | 1.20 | 32 |
| 10 | 43.34 | 1.02 | | 43.67 | 1.06 | | 43.67 | 1.01 | |
| 11 | 42.28 | 1.06 | | [symbol] | 1.07 | | 42.63 | 1.04 | |
| 12 | 41.22 | 1.06 | | 41.51 | 1.09 | | 41.58 | 1.05 | |
| 13 | 40.11 | 1.11 | 27 | 40.26 | 1.25 | 34 | 40.32 | [symbol] | 32 |
| 14 | 38.96 | 1.15 | | [symbol] | 1.15 | | 39.21 | 1.11 | |
| 15 | 37.80 | 1.16 | | 37.92 | 1.19 | | 38.06 | 1.15 | |
| 16 | 36.59 | 1.21 | | 36.66 | 1.26 | | 36.86 | 1.20 | |
| 17 | 35.41 | 1.18 | 29 | 35.38 | 1.28 | 34 | 35.60 | 1.26 | 33 |
| 18 | 34.21 | 1.20 | | 34.14 | 1.24 | | 34.39 | 1.21 | |
| 19 | 33.02 | 1.19 | | 32.89 | 1.25 | | 33.15 | 1.24 | |
| 20 | 31.80 | 1.22 | | 31.66 | 1.23 | | 31.91 | 1.24 | |
| 21 | 30.61 | 1.19 | 27 | 30.37 | 1.29 | 32 | 30.66 | 1.25 | 30 |
| 22 | | | | | | | | | |
| | Mean | 1.14 | | Mean | 1.18 | | Mean | 1.16 | |
| | % RSD | 7.3 | | % RSD | 8.1 | | % RSD | 8.0 | |
| | Total (g) | 23.87 | | Total (g) | 24.68 | | Total (g) | 24.32 | |
| | for 21 actuations | | | | | | | | |

| | AP35 - 31 g/3.1 g | | | AP35 - 31 g/2.4 g | | |
|---|---|---|---|---|---|---|
| | | Can | | | Can | |
| | Can | weight | Foam | Can | weight | Foam |
| Actuation | weight | difference | Height | weight | difference | Height |
| # | (g) | (g) | (mL) | (g) | (g) | (mL) |
| 0 | 54.61 | | | 54.19 | | |
| 1 | 53.50 | 1.11 | 24 | 53.00 | 1.19 | 26 |
| 2 | 52.20 | 1.30 | | 51.78 | 1.22 | |
| 3 | 50.97 | 1.23 | | 50.58 | 1.20 | |
| 4 | [symbol] | 1.17 | | 49.45 | 1.13 | |
| 5 | 48.64 | 1.16 | 32 | [symbol] | 1.12 | 21 |
| 6 | 47.59 | 1.05 | | 47.33 | 1.00 | |
| 7 | [symbol] | 1.00 | | 46.33 | 1.00 | |
| 8 | 45.43 | 1.16 | | 45.27 | 1.06 | |
| 9 | 44.22 | 1.21 | 32 | 44.32 | 0.95 | 19 |
| 10 | 43.18 | 1.04 | | 43.34 | 0.98 | |
| 11 | [symbol] | 1.04 | | 42.23 | 1.11 | |
| 12 | 41.09 | 1.05 | | 41.08 | 1.15 | |
| 13 | [symbol] | 1.23 | 31 | 40.04 | 1.04 | 20 |
| 14 | 38.74 | 1.12 | | 38.85 | 1.19 | |
| 15 | 37.55 | 1.19 | | 37.67 | 1.18 | |
| 16 | 36.37 | 1.18 | | 36.47 | 1.20 | |
| 17 | 35.14 | 1.23 | 32 | 35.55 | 0.92 | 16 |
| 18 | 33.95 | 1.19 | | 34.58 | 0.97 | |
| 19 | 32.73 | 1.22 | | [symbol] | 1.01 | |
| 20 | 31.54 | 1.19 | | 32.47 | 1.10 | |
| 21 | 30.27 | 1.27 | 30 | 31.25 | 1.22 | 21 |
| 22 | | | | | | |
| | Mean | 1.16 | | Mean | 1.09 | |
| | % RSD | 7.2 | | % RSD | 9.0 | |
| | Total (g) | 24.34 | | Total (g) | 22.94 | |

[symbol]

indicates data missing or illegible when filed

From these results, the propellant AP35 was chosen as providing a more consistent actuation amount.

Additionally, the peptide concentrations were further revised from the initial 30 μg/dose and 3000 μg/dose to the final concentrations 100 μg/dose and 300 μg/dose. With the decision to use AP35 propellant.

The following components were used for the informal stability study. These will be the basic packaging components used for the product in further development: Can, 30 g 35 mm×65 mm; Valve, CV/1″ Nitrile AR/PP; Actuator, Metered Head 1.35 mL; Foam Shield, Lablabo.

The revised base formulation for the pharmaceutical rectal foam composition is shown below in Table 69.

TABLE 69
Enhanced pharmaceutical rectal foam composition. The optimized formula based on the informal
stability and the formulation enhancement for Prototype A. The % w/w for the Purified Water
in the Active formulae is adjusted based on the desired active concentration. The amount
of API added per batch will be adjusted based on the Lot's purity and its water content.

| | Placebo | | | 100 μg dose | | | 300 μg dose | | |
|---|---|---|---|---|---|---|---|---|---|
| | Formula Code | | | | | | | | |
| | PDGW-1 | | | DGW-1 | | | DGX-1 | | |
| | % w/w | % w/w | μg/ | % w/w | % w/w | μg/ | % w/w | % w/w | μg/ |
| Ingredient | Bulk | Can | actuation | Bulk | Can | actuation | Bulk | Can | actuation |
| Purified Water | 77.5495 | 69.8847 | 803674 | 77.5399 | 69.8761 | 803575 | 77.5207 | 69.8587 | 803376 |
| USP/EP | | | | | | | | | |
| Propylene Glycol | 10.0000 | 9.0116 | 103634 | 10.0000 | 9.0116 | 103634 | 10.0000 | 9.0116 | 103634 |
| USP | | | | | | | | | |
| Sodium | 0.1640 | 0.1478 | 1700 | 0.1640 | 0.1478 | 1700 | 0.1640 | 0.1478 | 1700 |
| Phosphate | | | | | | | | | |
| Dibasic USP | | | | | | | | | |
| Sodium | 0.1165 | 0.1050 | 1207 | 0.1165 | 0.1050 | 1207 | 0.1165 | 0.1050 | 1207 |
| Dihydrogen | | | | | | | | | |
| Phosphate | | | | | | | | | |
| Monohydrate | | | | | | | | | |
| USP | | | | | | | | | |
| Disodium EDTA | 0.0500 | 0.0451 | 518 | 0.0500 | 0.0451 | 518 | 0.0500 | 0.0451 | 518 |
| USP | | | | | | | | | |
| Methylparaben | 0.1000 | 0.0901 | 1036 | 0.1000 | 0.0901 | 1036 | 0.1000 | 0.0901 | 1036 |
| NF | | | | | | | | | |
| Light Mineral Oil | 6.0000 | 5.4070 | 62180 | 6.0000 | 5.4070 | 62180 | 6.0000 | 5.4070 | 62180 |
| NF | | | | | | | | | |
| Isopropyl | 0.5000 | 0.4506 | 5182 | 0.5000 | 0.4506 | 5182 | 0.5000 | 0.4506 | 5182 |
| Myristate NF | | | | | | | | | |
| White Petrolatum | 1.0000 | 0.9012 | 10363 | 1.0000 | 0.9012 | 10363 | 1.0000 | 0.9012 | 10363 |
| USP | | | | | | | | | |
| Polyoxyl 20 | 2.5000 | 2.2529 | 25908 | 2.5000 | 2.2529 | 25908 | 2.5000 | 2.2529 | 25908 |
| Cetostearyl Ether | | | | | | | | | |
| NF | | | | | | | | | |
| Cetyl | 1.0000 | 0.9012 | 10363 | 1.0000 | 0.9012 | 10363 | 1.0000 | 0.9012 | 10363 |
| Alcohol | | | | | | | | | |
| NF | | | | | | | | | |
| Stearyl Alcohol | 1.0000 | 0.9012 | 10363 | 1.0000 | 0.9012 | 10363 | 1.0000 | 0.9012 | 10363 |
| NF | | | | | | | | | |
| Propylparaben | 0.0200 | 0.0180 | 207 | 0.0200 | 0.0180 | 207 | 0.0200 | 0.0180 | 207 |
| NF | | | | | | | | | |
| API (adj for | | | | 0.00961 | 0.0087 | 100 | 0.02883 | 0.0260 | 300 |
| potency)* | | | | | | | | | |
| Total | 100.00 | | | 100.00 | | | 100.00 | | |
| AP35 | | 9.8837 | 113663 | | 9.8837 | 113663 | | 9.8837 | 113663 |
| Propellant | | | | | | | | | |
| Total | | 100.00 | 1150000 | | 100.00 | 1150000 | | 100.00 | 1150001 |

*Target amounts reported. The amount of material added will be adjusted based on the API purity and water content.

Additional lab batches for the pharmaceutical rectal foam composition were produced for Microbial Limits Assay Validation. The Placebo, 100 μg/dose and 300 μg/dose batches produced based on the formula in Table 67 above. The Microbial Limits Assay Validation is shown in Tables 68-70 below.

TABLE 70
Microbial Limits Assay Validation. Pharmaceutical rectal
foam composition - Placebo. Lot 1820-0525501 (tested in triplicate).
(61) MICROBIOLOGICAL EXAMINATION OF NONSTERILE PRODUCTS:
MICROBIAL ENUMERATION TESTS

| | | | Total Yeasts |
|---|---|---|---|
| | | | and Molds |
| | | Total Aerobic Microbial Count | Count |
| Method | Organisms | (MCTA) | (SDA) |
| 74.8000 | E. coli ATCC 8739 | 1:10 | |
| | P. aeruginosa ATCC 9027 | 1:10 | |
| | S. aureus ATCC 6538 | 1:10 | |
| | B. subtilis ATCC 6633 | 1:10 | |
| | C. albicans ATCC 10231 | 1:10 | 1:10 |
| | A. brasiliensis ATCC 16404 | 1:10 | 1:10 |

Suitability method is equivalent to USP Microbial Enumeration Tests in USP general chapter <61>

TABLE 71
Microbial Limits Assay Validation. Pharmaceutical rectal foam composition - 100 μg.
Lot 1820-0525501 (tested in triplicate).
(61) MICROBIOLOGICAL EXAMINATION OF NONSTERILE PRODUCTS:
MICROBIAL ENUMERATION TESTS

| | | Total Aerobic | Total Yeasts and |
|---|---|---|---|
| | | Microbial Count | Molds Count |
| Method | Organisms | (MCTA) | (SDA) |
| 74.8000 | E. coli ATCC 8739 | 1:10 | |
| | P. aeruginosa ATCC 9027 | 1:10 | |
| | S. aureus ATCC 6538 | 1:10 | |
| | B. subtilis ATCC 6633 | 1:10 | |
| | C. albicans ATCC 10231 | 1:10 | 1:10 |
| | A. brasiliensis ATCC 16404 | 1:10 | 1:10 |

Suitability method is equivalent to USP Microbial Enumeration Tests in USP general chapter <61>

TABLE 72
Microbial Limits Assay Validation. Pharmaceutical rectal foam composition - 300 μg.
Lot 1820-0525501 (tested in triplicate).
(61) MICROBIOLOGICAL EXAMINATION OF NONSTERILE PRODUCTS:
MICROBIAL ENUMERATION TESTS

| | | Total Aerobic | Total Yeasts and |
|---|---|---|---|
| | | Microbial Count | Molds Count |
| Method | Organisms | (MCTA) | (SDA) |
| 74.8000 | E. soli ATCC 8739 | 1:10 | |
| | P. aeruginosa ATCC 9027 | 1:10 | |
| | S. aureus ATCC 6538 | 1:10 | |
| | B. subtilis ATCC 6633 | 1:10 | |
| | C. albicans ATCC 10231 | 1:10 | 1:10 |
| | A. brasiliensis ATCC 16404 | 1:10 | 1:10 |

Suitability method is equivalent to USP Microbial Enumeration Tests in USP general chapter <61>

Based on the Microbial Limits Assay Validation results shown in Tables 68-70, it was determined that the enumeration portion of this method was suitable for these test organisms; the method is suitable for enumerating microorganisms which may be contaminating the product.

For pharmaceutical rectal foam composition, Table 71 below provides the proposed physical specifications for Prototype A, pharmaceutical rectal foam composition. For the foam actuation weight, the amount observed on stability was 1.2 g per actuation. These values were obtained using AP-70. After the optimization study with the various propellants and changing to AP-35 the values obtained were observed for foam actuations to be 1.15 g±15%.

TABLE 73
Proposed physical specifications for pharmaceutical rectal foam composition.

| Specification | Bulk Product | Finished Product | |
|---|---|---|---|
| Appearance | White to off-white thin, | White to off-white foam | |
| | liquid emulsion | | |
| Specific gravity | Report Results | | |
| pH | 6.5-7.5 (neat) | 6.5-7.5 (1 actuation dissolved in | |
| | | 100 g of 5% Methanol/95% | |
| | | Water Solution | |
| Peptide API | 90.0-110.0% L | 90.0-110.0% L | |
| Methylparaben | 90.0-110.0% F | 90.0-110.0% F | |
| Propylparaben | 90.0-110.0% F | 90.0-110.0% F | |
| Foam Height (mL) | | NLT 20 | mL |
| Weight per Actuation | | 1.15 g ± 15% | |
| Duration of Expansion | | ≥1 | min |
| Total Aerobic Micro | | NMT 200 | CFU/mL |
| Total Yeast/Molds | | NMT 20 | CFU/mL |

| | |
|---|---|
| Objectionable Organisms | Absent |

Table 74 below provides the inactive ingredients and excipient limits and role or function these are used in the formula for Prototype A.

TABLE 74
Inactive ingredient and excipient role limit in pharmaceutical
rectal foam composition Prototype A.

| | | | | Finalized IID Limits | |
|---|---|---|---|---|---|
| Prototype A | % w/w | | | | Compared |
| Ingredients | Bulk | mg/Actuation | Function | IID Limit | Dosage form |
| Purified Water | 77.5495 | 807 | Solvent/Diluent | NA | NA |
| USP/EP | | | | | |
| Propylene Glycol | 10 | 104 | Diluent | 1214 mg | Rectal Aerosol |
| USP | | | | MDE | Foam |
| Sodium Phosphate | 0.164 | 1.71 | pH modifier | 38.25 mg | Intravesical |
| Dibasic USP | | | | per unit | solution¹, 6.48 |
| | | | | dose¹, 6.48 | mg/mL for nasal |
| | | | | mg/mL¹ | Spray¹ |
| Sodium | 0.1165 | 1.21 | pH modifier | Vaginal | In vaginal cream |
| Dihydrogen | | | | Cream QS to | to adj pH, Qty not |
| Phosphate | | | | adjust pH^{1, 2} | stated^{1, 2}. |
| Monohydrate USP | | | | 31.72 mg | Oral tablet¹ |
| | | | | per unit | |
| | | | | dose¹ | |
| Disodium EDTA | 0.05 | 0.52 | Chelating agent | 2 mg MDE | Rectal Aerosol |
| USP | | | | | Foam |
| Methylparaben | 0.1 | 1.04 | Preservative | 0.23% w/v | Rectal Suspension |
| NF | | | | | |
| Light Mineral Oil | 6 | 62.5 | Solvent | 1464.9 mg | Vaginal |
| NF | | | | per unit | Suppository¹ |
| | | | | dose¹ | |
| Isopropyl | 0.5 | 5.2 | Co-Solvent | 1% w/w¹ | Vaginal Cream¹ |
| Myristate NF | | | | | |
| White Petrolatum | 1 | 10.4 | Co-Solvent | 75% w/w | Rectal Ointment |
| USP | | | | | |
| Polyoxyl 20 | 2.5 | 26 | Emulsifier | 5.2% w/w¹ | Topical Aerosol |
| Cetostearyl Ether | | | | | Foam¹ |
| NF | | | | | |
| Cetyl Alcohol NF | 1 | 10.4 | Emulsifier | 17 mg MDE | Rectal Aerosol |
| | | | | | Foam |
| Stearyl Alcohol | 1 | 10.4 | Stiffening | 8.5% w/w¹ | Vaginal Cream¹ |
| NF | | | agent | | |
| Propylparaben NF | 0.02 | 0.21 | Preservative | 0.02% w/w | Rectal Enema |
| Total | 100 | 1041 | | | |
| AP35 Propellant | NA | 109 | Propellant | 9 mg | Rectal Aerosol |
| | | | | Propane | Foam |
| | | | | 12 mg | |
| | | | | Butane | |
| | | | | 142 mg | |
| | | | | isobutane | |
| | Total | 1150 | | | |

footnotes:
MDE—maximum Daily exposure
% w/w or % w/v is maximum potency per unit dose
¹No rectal formulation data available, other comparator dosage form like vaginal, intravesical, nasal, oral, topical used in such situation
²Compared to anhydrous since monohydrate listing not available

In conclusion, this example shows the informal stability and evaluation for the pharmaceutical rectal foam compositions Prototypes A and B. Here, the following outcomes have been reached: Based on the informal stability results, Prototype A was chosen for continued development.

The present example shows that in some embodiments, a temperature of 5° C. can be used for long term storage (>30 days). Likewise, in other embodiments, a storage temperature of about 25° C. can be used and is acceptable for short periods of time (not more than 60 days total).

Formulation enhancements to Prototype A show that AP35 can be used as a propellant for the product. The product was further refined to provide the concentrations of 100 μg/dose and 300 μg/dose in a 30-g aluminum aerosol can with a metered dose valve. Based on the actuation data using AP35 for Prototype A, it is suggested that the first actuation dose be used to prime and discarded leaving 20 potential clinical doses per can.

### Example 26. Clinical Trial Summary

The present example contemplates a phase 2 randomized, double-blind, placebo-controlled, adaptive study to evaluate the efficacy, safety, and tolerability of 2 dose levels of the peptide of the present disclosure, when administered rectally for 12 weeks to treat bladder pain in subjects with interstitial cystitis/bladder pain syndrome

The aim of this study is to assess the efficacy, safety, and tolerability of the peptide of the present disclosure for the treatment of Interstitial Cystitis/Bladder Pain Syndrome (IC/BPS). IC/BPS is a chronic condition characterized by bladder pain that is usually accompanied by urinary urgency, increased frequency, and/or nocturia. The morbidity of IC/BPS is substantial and often leads to poor quality of life for patients, their families, and day-to-day caregivers. See Mullins et al. Novel research approaches for interstitial cystitis/bladder pain syndrome: thinking beyond the bladder. Transl Androl Urol. 2015; 4(5):524-33.

It is estimated that 3% to 7% of women and 3% to 4% of men have IC/BPS. See Hanno et al. Diagnosis and treatment of interstitial cystitis/bladder pain syndrome: AUA guideline amendment. J Urol. 2015; 193(5):1545-53. At this time, there are no effective treatments targeting bladder pain in IC/BPS. A safe and effective treatment would greatly improve the quality of life for this patient population. The table below shows the objectives, Estimands, and endpoints of the clinical trial.

TABLE 75
Objectives, estimands, and endpoints of the clinical trial.

| | | |
|---|---|---|
| Objectives | Estimands | Endpoints |

Primary

| | | |
|---|---|---|
| Investigate the | The effect of the | Change from baseline in weekly average of daily bladder |
| efficacy of the | peptide of the | pain overall (which can include sensations like burning, |
| peptide of the | present disclosure | pressure, and/or discomfort) at its worst at Week 12 |
| present disclosure | compared to | |
| administered as a | placebo at | |
| rectal foam | Week 12 in | |
| (pharmaceutical | weekly average | |
| rectal foam | of daily bladder | |
| composition) on | pain at its worst | |
| bladder pain in | scores | |
| subjects with | | |
| IC/BPS | | |

Secondary

| | | |
|---|---|---|
| Investigate the | — | Change from baseline in weekly average of a |
| efficacy of the | | burning sensation in the bladder at its worst at |
| peptide of the | | Week 12 |
| present disclosure | | Change from baseline in weekly average of a |
| administered as a | | pressure sensation in the bladder at its worst at |
| rectal foam on the | | Week 12 |
| components of | | Change from baseline in weekly average of |
| bladder pain in | | discomfort in the bladder at its worst at Week 12 |
| subjects with | | Change from baseline in Genitourinary Pain Index |
| IC/BPS | | (GUPI) Pain subscale score at Week 12 |
| Investigate the | — | Frequency of treatment-emergent adverse events |
| safety and | | (TEAEs) occurring in ≥2% of subjects |
| tolerability of the | | Overall frequency of TEAEs by severity grade |
| peptide of the | | |
| present disclosure | | |
| administered as a | | |
| rectal foam in | | |
| subjects with | | |
| IC/BPS | | |

Exploratory

| | |
|---|---|
| — | Weekly average change from baseline in daily |
| | bladder pain at its worst |
| | Weekly average change from baseline in daily |
| | bladder pain-free days |
| | Weekly average change from baseline in urinary |
| | urgency |
| | Weekly change from baseline in mean urinary |
| | frequency |
| | Weekly change from baseline in mean nighttime |
| | voiding frequency (nocturia) |
| | Change from baseline in O'Leary/Sant Interstitial |
| | Cystitis Symptom Index (ICSI) total score |
| | Change from baseline in Genitourinary Pain Index |
| | (GUPI) Urinary subscale score |
| | Change from baseline in GUPI Quality of life |
| | (QoL) subscale score |

Primary

Change from baseline in Global Response
Assessment (GRA) total score
Change from baseline in the Sexual Function
Question score
Change from baseline in sleep difficulty due to
bladder pain
Change from baseline in sleep difficulty due to
needing to urinate
Change from baseline in the body pain category as
assessed using the Margolis body map
Change from baseline in IC/BPS Symptom
Severity
IC/BPS global impression of symptom change
6/12 Week Adequate Relief of IC/BPS Pain
Responder
Change from baseline in EuroQol 5-Dimension 5-
Level Version (EQ-5D-5L) utility score and Visual
Analog Scale
Change from baseline in 16s RNA based analysis
of gut microbiome composition at the genus level
Pharmokinetic (PK) parameters of peptide of the
present disclosure and metabolites in plasma

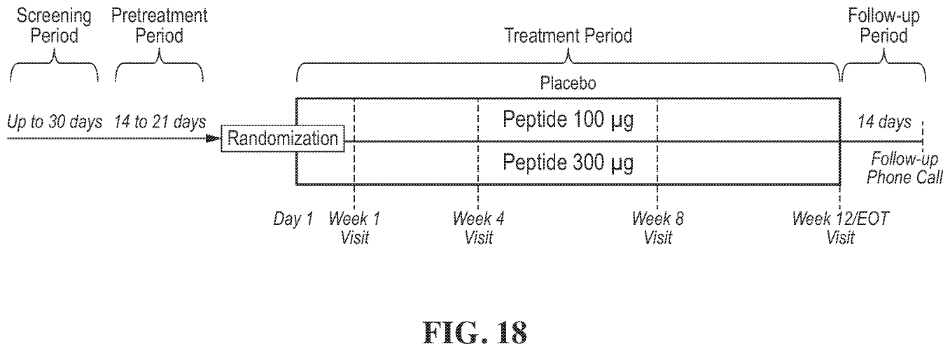

As shown in FIG. 18 the study consists of 4 periods: a Screening Period, a Pretreatment Period, a 12-week Treatment Period, and a 2-week Follow-up Period. The assessments conducted during each period are shown in the Schedule of Activities (SoA) table below. This is a 12-week, double-blind, parallel-group, 3-arm study to evaluate the efficacy, safety, and tolerability of 2 dose strengths of the peptide of the present disclosure (100 μg or 300 μg) or matching placebo, administered once daily as a rectal foam.

During the Screening Visit, subjects will undergo preliminary screening procedures to ensure that they meet the eligibility criteria for the study. Subjects requiring additional procedures or a medication washout will have up to 30 days to become eligible to enter the Pretreatment Period. The end of the Screening Period coincides with the beginning of the Pretreatment Period. During this period, subjects will receive training on how to complete the daily and weekly electronic diary (eDiary) questions at home. On subsequent days, subjects will answer the eDiary questions to establish a baseline. Eligibility to continue into the Treatment Period of the study will be based on the answers recorded in the eDiary during the 14 days prior to Day 1.

Subjects who meet the entry criteria will be randomized on Day 1 and continue into the Treatment Period. On Day 1 of the Treatment Period, randomized subjects will receive the first dose of study drug, formulated as a rectal foam and administered by the subject under supervision of the site staff. Subjects will also receive training on how to self-administer the study drug at home. For the next 12 weeks, subjects will self-administer study drug at approximately the same time each day. Subjects will also continue to answer the daily and weekly eDiary questions. Subjects will have routine study visits on Week 1, Week 4, Week 8, and Week 12/End of Treatment (EOT) for completion of efficacy, safety, and other assessments. Two weeks after the last day of dosing (Week 12/EOT Visit), an End of Study (EOS) Follow-up Phone Call will be conducted to assess the subject for AEs and medications taken since the Week 12/EOT Visit.

Approximately 300 subjects will be randomly assigned to study intervention in order to have approximately 255 evaluable subjects (85 per group). The sample size of ˜300 subjects represents the maximum number of subjects to be enrolled based on initial assumptions. Subjects will be randomized in a 1:1:1 ratio to receive 100 μg or 300 μg of the peptide of the present disclosure, or matching placebo. Study drug will be administered rectally once daily for 12 weeks.

The table showing the Schedule of Activities (SoA) is provided below.

TABLE 76
Schedule of Activities (SoA).

| | Study Period | | | | | | | |
|---|---|---|---|---|---|---|---|---|
| | Screening | Pretreatment | | | | | | Follow-up |
| | Period | Period | | | | | | Period |
| | (Up to 30 days) | (14 to 21 days) | Treatment Period (12 weeks) | | | | | (2 weeks) |
| | Visit/Day | | | | | | | |
| | | | | | | | | End of |
| | | | | | | | | Study |
| | | | | | | | | Follow-up |
| | | Pretreatment | | Week 1 | Week 4 | Week 8 | Week 12/EOT | Phone |
| Study | Screening Visit | Visit | Randomization | Visit | Visit | Visit | Visit | Call |
| Procedure | (Day −51 to | (Day −21 to | Visit/ | (Day | (Day | (Day | (Day | (Day |
| ↓ | Day −15) | Day −14) | Day 1 | 7 ± 3) | 28 ± 3) | 56 ± 3) | 84 + 3) | 98 ± 3) |
| Informed consent | X | | | | | | | |
| Eligibility criteria | X | X | X* | | | | | |
| IWRS registration | X | X | X** | X | X | X | X | X |
| Medical history and IC/BPS | X | | | | | | | |
| characteristics^{a} | | | | | | | | |
| Demographics | X | | | | | | | |
| Body weight & height^{b} | X | | X | X | X | X | X | |
| Urine alcohol and drug screen | X | | | | | | | |
| HIV antibody and hepatitis panel | X | | | | | | | |
| Physical examination^{c} | X | | X | X | X | X | X | |
| Visual rectal examination^{d} | X | | X | X | X | X | X | |
| Digital rectal exam | X | | | | | | X | |
| Seated vital signs^{e} | X | | X* | X | X | X | X | |
| 12-lead ECG | X | | Post: 1 h^{f} | | | | X | |
| Clinical chemistry, hematology, | X | | X** | X | X | X | X | |
| urinalysis | | | | | | | | |
| ESR and CRP | | | X** | X | X | X | X | |
| Pregnancy test_{g} | X | | X* | | X | X | X | |
| Hemoccult test^{h} | X | | | | | | X | |
| Prohibited medications washout | X | | | | | | | |
| instructions | | | | | | | | |
| Prior and concomitant | X | X | X* | X | X | X | X | X |
| medications | | | | | | | | |
| AE monitoringⁱ | X | X | X | X | X | X | X | X |
| eDiary trainingⱼ | | X | | | | | | |
| Daily and weekly eDiary | | X | | | | | | |
| assessments^{k} | | | | | | | | |
| GUPI (weekly) | | | X** | X | | | | |
| GRA (weekly) | | | | X | | | | |
| Monthly eDiary assessment | | | X** | | X | X | X | |
| O'Leary/Sant ICSI^{l} | | | | | | | | |
| EQ-5D-5L^{m} | | | X** | | X | X | X | |
| HADSⁿ | | | X** | | | | X | |
| Margolis body map for pain | | | X** | | X | X | X | |
| assessment^{o} | | | | | | | | |
| Compliance verification and | | | X* | X | X | X | X | |
| reminder for the eDiaryₚ | | | | | | | | |
| Randomization | | | X** | | | | | |
| Rescue medicine dispensed | | X | | | | | | |
| Study drug dispensed | | | X | | X | X | | |
| Study drug administration training | | | X** | | | | | |
| Study drug administration_{q} | | | | | X | | | |
| PK blood draws^{r} | | | Pre: 0 | | X | X | X | |
| | | | (≤15 m) | | | | | |
| | | | Post: 1 h | | | | | |
| | | | (±15 m) | | | | | |
| Stool collection^{s} | | | X | | | | X | |
| Study drug accountability^{t} | | | | X | X | X | X | |
| Follow-up phone call^{u} | | | | | | | | X |

AE = adverse event; CRP = c-reactive protein; ECG = electrocardiogram; eCRF = electronic case report form; eDiary = electronic diary; EOT = end of treatment; EQ-5D-5L = EuroQol 5-Dimension 5-Level; ESR = erythrocyte sedimentation rate; GRA = Global Response Assessment; GUPI = Genitourinary Pain Index; HADS = Hospital Anxiety and Depression Scale; ICF = informed consent form; ICSI = Interstitial Cystitis Symptom Index; IWRS = interactive web response system; PK = pharmacokinetic; Post = postdose; Pre = predose; WOCBP = women of childbearing potential.
*Assessment done prior to randomization;
**Assessment done predose
^{a}Site staff will complete the Prior IC/BPS Symptom Management Assessment
^{b}Height will only be measured at the Screening Visit.
^{c}A complete physical examination at Screening. At all other visits, physical examinations will be symptom-directed.
^{d}The visual rectal examination is a visual inspection of the perianal area. If significant redness, rectal pain, bleeding, or mucus is observed on visual inspection of the rectal (perianal) area, a digital rectal examination and hemoccult test should be performed. If further investigation is warranted, subjects with these signs or symptoms should undergo proctoscopy or recto-sigmoidoscopy. If these tests do not identify the source of inflammation, a colonoscopy may be performed after a discussion between the investigator and the sponsor.
^{e}Vital signs must be obtained in the seated position and will include oral temperature, respiratory rate, blood pressure, and pulse rate. Blood pressure and pulse measurements will be taken after the subject has been sitting for 5 minutes in a quiet setting without distractions (e.g., television, cell phones) and prior to blood draws, when applicable.
^{f}ECGs should be done after the subject has been supine for at least 5 minutes and prior to blood draws, when applicable.
_{g}Required for WOCBP. Serum-based test at Screening and Week 12/EOT. Urine-based test at all other time points, with a repeat serum-based test if the urine-based test is positive.
^{h}Stool that is present on the digital rectal exam can be used for the hemoccult test.
ⁱAll AEs occurring after the subject signs the ICF will be captured.
ⱼAt the Pretreatment Visit, the study coordinator will train the subject on use of the eDiary. Refer to the eDiary user manual.
^{k}The subject will complete the eDiary, responding to the daily questions and weekly GUPI and GRA questionnaires.
^{l}The subject will complete the questionnaire via the eDiary.
^{m}The subject will complete the questionnaire via the eDiary.
ⁿThe subject will complete the questionnaire via the eDiary.
^{o}The subject will complete the assessment at the site.
ₚAt the Day 1 Visit and all subsequent visits, the study coordinator will review the eDiary entries to verify subject compliance with the requirements. After determining the subject's compliance, the study coordinator will remind subjects to complete the eDiary (except at the Week 12/EOT Visit).
_{q}At the Day 1 Visit, study drug will be administered in the clinic by the subject under supervision of the site staff. On all subsequent days through the Week 12 study visit, the subject will self-administer study drug at home.
^{r}On Weeks 4, 8, and 12, a single PK sample will be drawn during the site visit without respect to the timing of the previous dose administration. The time of the subject's last dose will be recorded.
^{s}Subjects in the microbiome substudy will bring in a stool sample using the sample collection kit issued by the site.
^{t}Study drug accountability will be performed as described herein.
^{u}Appropriate site personnel will contact subjects by phone for safety follow-up. Any AE or concomitant medication reported during the phone call will be captured on the eCRF. Subjects who report AEs during the call will be provided with instructions on the appropriate follow-up care. At the discretion of the investigator, subjects may be requested to return to the clinic for their follow-up contact.

### Example 27. Clinical Trial Study Design

### Overall Design

The present disclosure contemplates a Phase 2, randomized, placebo-controlled, parallel-assignment, 12-week, adaptive-design study to evaluate the efficacy, safety, and tolerability of the peptide of the present disclosure—administered as a pharmaceutical rectal foam composition—in subjects having IC/BPS. Subjects will be randomized 1:1:1 to 100 μg, 300 μg, or matching placebo administered as a rectal foam. As shown in FIG. 18, the study consists of 4 periods: a Screening Period, a Pretreatment Period, a 12-week Treatment Period, and a 2-week Follow-up Period.

The Screening Period will begin with the signing of the informed consent form (ICF) at the Screening Visit (which can occur from Day −51 to Day −15) and will last up to 30 days. During this time, subjects will undergo preliminary procedures to ensure that they meet the eligibility criteria for the study. Sites with supporting procedures in place may elect to obtain informed consent electronically and conduct initial assessments that do not require the subject to be present (e.g., collecting demographics, medical history, prior medications) over the phone. Subjects requiring additional procedures, or a medication washout will have up to 30 days to become eligible to enter the Pretreatment Period.

The end of the Screening Period coincides with the beginning of the Pretreatment Period. The Pretreatment Period is defined as the 14 to 21 days immediately before the Day 1/Randomization Visit. At the Pretreatment Visit, subjects will receive training on how to complete the online daily and weekly eDiary questions at home. Sites with supporting procedures in place may elect to perform this visit virtually using a video telehealth system. Subjects will answer the eDiary questions throughout the Pretreatment Period to establish a baseline. Eligibility to continue into the Treatment Period of the study will be based on the answers recorded in the eDiary during the 14 days prior to Day 1. Subjects who meet the entry criteria will be randomized on Day 1 and continue into the Treatment Period.

On Day 1 of the Treatment Period, randomized subjects will receive the first dose of study drug, formulated as a rectal foam and administered by the subject under supervision of the site staff. Subjects will also receive training on how to self-administer the study drug at home. For the next 12 weeks, subjects will self-administer study drug at approximately the same time each day. Subjects will also continue to answer the daily and weekly eDiary questions.

Subjects will have routine study visits on Week 1, Week 4, Week 8, and Week 12/End of Treatment (EOT) for completion of efficacy, safety, and other assessments. Two weeks after the last day of dosing (Week 12/EOT Visit), an End of Study (EOS) Follow-up Phone Call will be conducted to assess the subject for any AEs and any medications taken since the Week 12/EOT Visit.

### Scientific Rationale for Study Design

A randomized, double-blind, placebo-controlled study design was chosen in accordance with the concepts in ICH E10 (Choice of Control Groups and Related Issues in Clinical Trials) to investigate the effects of the peptide of the present disclosure administered once daily for 12 weeks as a rectal foam. Subjects will be randomized to ensure that the treatment groups are comparable and to minimize the potential for selection bias. The study will be double-blind to ensure that the subjects and site staff are unaware of the treatment assignment and to minimize the potential for bias in study assessments or Adverse Event (AE) reporting. Placebo was chosen as the control so that the rate of spontaneously occurring AEs can be determined and to reduce the potential for bias in the reporting of AEs. This study has a 14- to 21-day Pretreatment Period to establish a baseline and to familiarize subjects with data collection methodology (i.e., eDiary), and a 12-week Treatment Period to compare the peptide of the present disclosure with a matching-placebo control.

Subject Input into Design

Patients with IC/BPS were surveyed to gain insights into the burden of disease and the appropriateness of select aspects of the study design including endpoints, assessments, and eligibility criteria.

### Justification for Dose

The peptide of the present disclosure doses of 100 μg and 300 μg were selected based on nonclinical pharmacology models of visceral hypersensitivity and bladder dysfunction using the peptide of the present disclosure and another GC-C agonist, linaclotide, with similar activity. The pharmacologically active doses of the peptide of the present disclosure in these nonclinical models are in the range of 3 μg/kg/day to 10 μg/kg/day, with diminishing benefit observed with doses above 10 μg/kg/day. The human equivalent daily dose (HED) extrapolated from pharmacologically active dose levels in nonclinical models is 180 μg to 600 μg (based on body weight) and 15 μg to 100 μg based on body surface area. Choosing 2 doses for investigation in this study should allow for observation of a dose response.

### End of Study Definition

A subject is considered to have completed the study if they have completed all phases of the study including the EOS/Follow-up Phone Call. The end of the study is defined as the date of the last EOS/Follow-up Phone Call of the last subject in the study.

### Example 28. Clinical Trial Study Population

Prospective approval of protocol deviations to recruitment and enrollment criteria, also known as protocol waivers or exemptions, is not permitted. Subjects are eligible to be included in the study only if all of the following criteria apply:

Age: Subject is 18 to 70 years of age, inclusive, at the time of signing the ICF.

Type of Subject and Disease Characteristics: Subject has been diagnosed with IC/BPS at least 6 months prior to entering the study and currently meets the American Urological Association (AUA) criteria for IC/BPS. Subject has chronic bladder pain associated with filling and emptying the bladder over the past 6 months. Subject has bladder pain overall (which can include sensations like burning, pressure, and/or discomfort) on at least 10 days and an average daily score for bladder pain at its worst ≥4, as reported in the eDiary using an 11-point numeric rating scale (NRS) during the 14 days before the Day 1 Visit. Subject has at least 1 other urinary symptom related to IC/BPS (i.e., nocturia ≥2 voids/night, daytime frequency ≥8×day) during the 3 months before the Screening Visit. Subject is compliant with eDiary completion by adequately responding to eDiary questions (i.e., completing the daily evening report) at least 10 days over Week-1 and Week-2 (14-day period) with at least 5 of those eDiary completion days occurring during Week-1. Subject has a weekly average daily score for bladder pain at its worst of ≥4, as reported in the eDiary using an 11-point numeric rating scale (NRS) during Week-1. Subject has at least 1 of the following urinary symptoms related to IC/BPS during the 3 months before the Screening Visit:
nocturia ≥2 voids/night
daytime frequency >8×day
urgency

Subject is willing to use a rectally-administered product and able to administer study drug, understand directions, and complete study assessments. If the subject has been treated with nonpharmacologic interventions for IC/BPS (e.g., physical therapy, pelvic floor massage, acupuncture, naturopathy, mindfulness exercises, cognitive behavioral therapy), these were unchanged for a minimum of 30 days before the Screening Visit and the subject agrees to continue them unchanged from the Screening Visit until after the EOS Follow-up Phone Call. Subject agrees to refrain from making any new or major lifestyle changes that may affect IC/BPS symptoms (e.g., new diet, exercise regimen) from the Screening Visit until after the EOS Follow-up Phone Call. Subject agrees to comply with all other lifestyle restrictions.

Weight: Subject has a body mass index ≤40 kg/m² at the Screening Visit.

Sex: a female subject is eligible to participate if she is not pregnant or breastfeeding, and one of the following conditions applies: (a) Is a woman of nonchildbearing potential (WONCBP) as defined in Contraceptive and Barrier Guidance; or (b) Is a women of childbearing potential (WOCBP), who is using a contraceptive method that is highly effective, with a failure rate of <1%. The investigator should evaluate the potential for contraceptive method failure (e.g., noncompliance, recently initiated) in relationship to the first dose of study drug. Additionally, a female who has a negative pregnancy test before the first dose of study intervention on Day 1, refer below (Pregnancy Testing). If a urine test cannot be confirmed as negative (e.g., an ambiguous result), a serum pregnancy test is required. In such cases, the subject must be excluded from participation if the serum pregnancy result is positive.

A male subject is eligible to participate if he agrees to the following during the study (i.e., from the Pretreatment Visit until the EOS Follow-up Phone Call): (a) Refrain from donating sperm, PLUS, either: (b) Be abstinent from heterosexual intercourse as their preferred and usual lifestyle (abstinent on a long term and persistent basis) and agree to remain abstinent; or (c) Agree to use contraception/barrier; Agree to use a male condom and should also be advised of the benefit for a female partner to use a highly effective method of contraception as a condom may break or leak when having sexual intercourse with a woman of childbearing potential who is not currently pregnant. Agree to use a male condom when engaging in any activity that allows for passage of ejaculate to another person. Also, Subject agrees to refrain from inserting anything other than the study drug applicator into the rectum from the Screening Visit until after the EOS Follow-up Phone Call outlined below.

Informed Consent: Subject is capable of giving signed informed consent as described below, which includes compliance with the requirements and restrictions listed in the ICF and in this protocol.

### Exclusion Criteria

Subjects are excluded from the study if any of the following criteria apply:

Medical Conditions: Male subject has a history of prostatitis or benign prostatic hyperplasia. Subject has pelvic floor dysfunction or vulvodynia that, in the opinion of the investigator, would be difficult to discriminate from bladder pain. Subject has anal fissure, anal abscess, inflammatory bowel disease, active proctitis, or any other condition that can be a contraindication to using a rectal foam or bias the evaluation of efficacy and safety of the product. Subject has cancer under active treatment or a history of uterine, cervical, pelvic, rectal, ovarian, or vaginal cancer. Subject has a history of benign or malignant bladder tumors. Subject has an unresolved urinary tract infection during the Screening or Pretreatment Period. Subject has a positive hemoccult test at the Screening Visit. Subject has a clinically significant concurrent illness or finding on physical examination or clinical laboratory test after signing the ICF but before receiving the first dose of study drug. (The investigator will determine if a finding is clinically significant. The investigator will consider whether the finding 1) could prevent the subject from performing any study procedure or assessment, 2) represents a condition that would be exclusionary, 3) could represent a safety concern if the subject participated in the study, or 4) could confound any study assessment.) Subject has a hypersensitivity or allergy to any of the ingredients contained in the peptide drug product. Subject has an uncontrolled major psychiatric condition or has made a suicide attempt during the 2 years before the Screening Visit. Subject has a malabsorption syndrome.

Prior/Concomitant Therapy: Subject has any history of surgery in the pelvic region or has had surgery in the abdominal region within 90 days of the Screening Visit. Subject has received a cystoscopy (with or without hydrodistension for reason other than pain relief) for diagnostic purposes within 30 days before the Screening Visit or a cystoscopy involving therapeutic hydrodistension (for pain relief) within 90 days before the Day 1 Visit. Subject has history of pelvic irradiation or radiation cystitis. Subject is taking or has recently taken a prohibited medication or is not willing to abide by the restrictions outlined below. Subject has a positive drug screen. A positive result for amphetamines is allowed if the subject has a documented medical need (refer below); a positive result for marijuana is also allowed.

Prior/Concurrent Clinical Study Experience: Subject has participated in another interventional clinical study within 6 months of the Screening Visit or is planning to do so at any time during the study.

Other Exclusions: Subject has an acute or chronic condition that, in the investigator's opinion, would limit their ability to complete or participate in the study. Subject has a recent history (during the 12 months before the Day 1 Visit) of drug or alcohol abuse. (Note: Subjects with a history of drug or alcohol abuse that was diagnosed greater than 12 months before the Day 1 Visit may be enrolled if they have exhibited no actual abuse during the 12 months before the Day 1 Visit.). Subject has previously entered the Pretreatment Period of this study or has already been enrolled in this study at another clinical site. Subject is directly or indirectly involved in the conduct and administration of this study as an investigator, subinvestigator, study coordinator, study staff member; or the subject is a direct relative of one of the above individuals involved in the study.

### Lifestyle Considerations

Meals and Dietary Restrictions: Subjects should not make substantial changes to their diet from the signing of the ICF until after the EOS Follow-up Phone Call.

Caffeine, Alcohol, and Tobacco: Subjects should not make any substantial changes to their consumption habits for caffeine, alcohol, or tobacco from the signing of the ICF until after the EOS Follow-up Phone Call.

Activity: If the subject is using any nonpharmacologic interventions for IC/BPS (e.g., physical therapy, pelvic floor massage, acupuncture, naturopathy, mindfulness exercises, or cognitive behavioral therapy), those should continue unchanged for a minimum of 30 days before the Screening Visit until after the EOS Follow-up Phone Call. Subjects must comply with the contraception requirements outlined below. Subject agrees to refrain from inserting anything other than the study drug applicator into the rectum from the Screening Visit until after the EOS Follow-up Phone Call.

Screen Failures: Screen failures are defined as subjects who consent to participate in the clinical study but are not subsequently randomized. A minimal set of screen failure information is required to ensure transparent reporting of screen failure subjects to meet the Consolidated Standards of Reporting Trials (CONSORT) publishing requirements and to respond to queries from regulatory authorities. Minimal information includes demography, screen failure details, eligibility criteria, and any SAE. Subjects who do not meet 1 or more study entry criteria may be rescreened following consultation with the sponsor. Subjects who fail screening due to Exclusion Criteria #6, the presence of an unresolved urinary tract infection, are allowed to rescreen without consultation with the sponsor. Rescreened subjects must be assigned a separate subject number for each screening; once a subject number has been assigned it must not be reused.

### Example 29. Clinical Trial Study Intervention

Study intervention (also referred to as study drug) is defined as any investigational intervention(s), and may include marketed product(s), placebo, intended to be administered to a study subject according to the study protocol.

The study will evaluate, the pharmaceutical rectal foam composition, 100 μg and 300 μg, and matching placebo in a double-blind manner. Study drug is formulated as an oil-in-water emulsion filled into an aluminum canister with a metered-dose head and an aerosol propellant for delivery as a foam (see Examples above). Briefly, each drug product canister is intended to be used for 22 actuations, 1 priming actuation followed by 21 dosing actuations. The active bulk rectal foam is formulated in 2 concentrations such that a single, metered actuation of 1.15 g±15% of foam corresponds to 1 of the following unit doses, 100 μg or 300 μg.

The pharmaceutical rectal foam composition, having a dose of about 100 μg and about 300 μg, contains the active pharmaceutical ingredient (i.e., peptide of the present disclosure) with the following excipients: purified water, propylene glycol, sodium phosphate dibasic, sodium dihydrogen phosphate monohydrate, disodium edetate (disodium EDTA), methylparaben, light mineral oil, isopropyl myristate, white petrolatum, polyoxyl 20 cetostearyl ether, cetyl alcohol, stearyl alcohol, propylparaben and AP-35. AP-35 is a commonly used aerosol propellant which is a mixture of butane, iso-butane, and propane. Matching placebo will be provided in identical canisters and contain the same excipients as the active formulation. Study drug will be administered once daily as a rectal foam for 12 weeks.

TABLE 77
Study interventions administered

| Intervention Name | Peptide 100 μg | Peptide 300 μg | Matching Placebo |
|---|---|---|---|
| Type | Drug | Drug | Placebo |
| Dose Formulation | Rectal Foam | Rectal Foam | Rectal Foam |
| Unit Dose | 100 μg/1.15 mL | 300 μg/1.15 mL | 0 μg/1.15 mL |
| Strength/Dose Volume | (foaming to 20 mL) | (foaming to 20 mL) | (foaming to 20 mL) |
| Dosage Level(s) | 100 μg once daily | 300 μg once daily | 0 μg once daily |
| Route of | Rectally | Rectally | Rectally |
| Administration | | | |
| Use | Experimental | Experimental | Placebo |
| Investigational | IMP | IMP | IMP |
| Medical Product | | | |
| (IMP)/ | | | |
| Noninvestigational | | | |
| medical product | | | |
| (NIMP) | | | |
| Packaging and | Study drug will be | Study drug will be | Study drug will be |
| Labeling | provided in canisters. | provided in canisters. | provided in canisters. |
| | Each canister will be | Each canister will be | Each canister will be |
| | labeled as required per | labeled as required per | labeled as required per |
| | country requirement. | country requirement. | country requirement. |

### Dosing Information

At the Day 1 Visit, study drug will be administered in the clinic by the subject under supervision of the site staff. On all other days during the Treatment Period, subjects will self-administer study drug at home. Subjects will continue dosing until their Week 12 study visit.

### Dose Administration at the Day 1 Site Visit

On Day 1, subjects will receive a single dose of study drug at the site visit. Subjects will be encouraged to empty their bowels in the morning prior to dosing, if possible. Before dosing, the site staff will provide the subject with study drug administration training and instructional materials for self-administration at home. Following training, subjects will administer study drug rectally as per the Directions for Use under the supervision of site staff. Subjects will be instructed to stand, with 1 foot on the floor and the other foot elevated on a flat surface. Subjects will place a prelubricated applicator onto the pump nozzle, shake the canister, unlock the pump dome, and invert the canister. They will then gently insert the applicator into the rectum as far as comfortable, press and hold the pump dome for approximately 5 seconds, then release the pump dome to administer the metered dose into the rectum (metered dose volume is 1.15 mL, which foams to a volume of approximately 20 mL). After administering the foam, subjects will hold the applicator in the rectum for 10 to 15 seconds, then dispose of the used applicator. Site staff will monitor subjects for leakage of the study drug from the rectum during the initial 30 minutes post-administration. Subjects should avoid using the bathroom and try to hold in the foam for at least 1 hour. After subjects complete the Day 1 postdose assessments shown in the SoA, they can leave the clinic.

The site staff will record the date and time of study drug administration in the electronic case report form (eCRF) and will also record (as yes/no) if there was study drug leakage from the rectum in the 30 minutes following drug administration.

### Dose Administration at Home

Following Day 1, subjects will self-administer study drug at home once daily at approximately the same time of day according to their routine. Subjects will be encouraged to empty their bowels prior to dosing, if possible. Dosing at night before bedtime is recommended. Dosing should be done in accordance with study drug administration training received at the Day 1 site visit.

For self-administration of study drug at home, subjects will be instructed to administer the foam in a standing position, with 1 foot on the floor and the other foot elevated. Subjects will place a prelubricated applicator onto the pump nozzle, shake the canister, unlock the pump dome, and invert. They will then gently insert the applicator into their rectum as far as comfortable, press and hold the pump dome for approximately 5 seconds, then release the pump dome to administer the metered dose into the rectum (metered dose volume is 1.15 mL, which foams to a volume of approximately 20 mL). After administering the foam, subjects will hold the applicator in their rectum for 10 to 15 seconds, then dispose of the used applicator. Subjects should avoid using the bathroom and try to hold in the foam for at least 1 hour. On a daily basis, subjects will complete the study drug retention and study drug application assessments in the eDiary.

### Packaging, Labeling, Storage, and Accountability

The pharmaceutical rectal foam composition, having a dose of about 100 μg and about 300 μg, and matching placebo, will be supplied in metered-dose canisters containing enough rectal foam for 22 actuations, 1 priming actuation followed by 21 dosing actuations. Each canister will be provided with a kit containing 21 prelubricated applicators and disposal bags for used applicators. Canisters will be uniquely numbered and labeled in a double-blind fashion that conforms to regulatory requirements.

The pharmaceutical rectal foam composition, 100 μg dose and 300 μg dose, and matching placebo canisters, will be shipped and stored at 2° C. to 8° C. (35° F. to 46° F.). Once the first dose from each canister has been administered, the subject may store the canister at ambient conditions for the remainder of its use. Any deviations from the storage conditions must be reported and use of the study drug suspended until authorization for its continued use has been provided.

Storage and accountability of study drug should adhere to the following: The investigator or designee must confirm appropriate temperature conditions have been maintained during transit for all study drug received and any discrepancies are reported and resolved before use of the study drug. Only subjects enrolled in the study may receive study drug and only authorized site staff may supply study drug or administer study drug at the Day 1 Visit. All study drug must be stored in accordance with the labeled storage conditions. The investigator, institution, or the head of the medical institution (where applicable) is responsible for study drug accountability, reconciliation, and record maintenance (i.e., receipt, reconciliation, and final disposition records).

### Measures to Minimize Bias: Randomization and Blinding

### Randomization

The subject identification (SID) number will be assigned in an ascending sequential order (e.g., beginning with 001, 002, etc.) at the time the subject signs the ICF. The subject will retain the same unique SID number throughout the Clinic and Follow-up Periods.

Subjects who meet all of the inclusion criteria and none of the exclusion criteria will be randomized into the study on Day 1. Approximately 300 subjects will be randomized in a 1:1:1 ratio to receive a dose of 100 μg (N=100), a dose of 300 μg (N=100), or matching placebo (N=100). Randomization numbers encoding the subjects' study intervention assignments will be based on a randomization schedule that is computer generated prior to the study by an independent statistician from a contract research organization (CRO) who is not otherwise associated with the study. All subjects will be centrally assigned to randomized study intervention using an Interactive Web Response System (IWRS). Before the study is initiated, the telephone number and call-in directions for the IWRS and/or the log in information and directions for the IWRS will be provided to each site.

### Blinding

The investigator and all other site staff, sponsor study personnel, and the subject will remain blinded to individual subject treatment assignments throughout the study, except as noted below. As described below, an independent DMC will conduct an IA of the safety and efficacy data. A designated independent study statistician, who will not be involved in study data analysis and interpretation, will have access to the randomization schedule including treatment assignments to conduct the IA. Specific designated personnel in the Ironwood Global Patient Safety Group may be unblinded to the treatment assignment of individual subjects for regulatory reporting purposes. All other sponsor study personnel, except as described, will remain blinded until the study is complete and the database is locked, unless warranted by emerging safety or tolerability issues. The handling of PK concentration data that may unblind subject treatment assignments is described below.

### Procedures for Unblinding

The IWRS will be programmed with blind-breaking instructions. In case of an emergency, the investigator has the sole responsibility for determining if unblinding of a subjects' intervention assignment is warranted. Subject safety must always be the first consideration in making such a determination. If the investigator decides that unblinding is warranted, the investigator should make every effort to contact the sponsor prior to unblinding a subject's intervention assignment unless this could delay emergency treatment of the subject. If a subject's intervention assignment is unblinded, the sponsor must be notified within 24 hours after breaking the blind. The date and reason that the blind was broken must be recorded in the source documentation and case report form, as applicable.

### Study Intervention Compliance

On Day 1, subjects will self-administer study drug under the supervision of designated site staff. The date and time of the dose administered in the clinic will be recorded in the source documents and recorded in the eCRF. The dose of study intervention and study subject identification will be confirmed at the time of dosing by a member of the study site staff other than the person administering the study intervention. Following Day 1, when subjects self-administer study drug at home, compliance with study intervention will be assessed at each visit. Compliance will be assessed (by weighing the returned canisters during the site visits and through subjects' reports in the daily eDiary [refer below]) and documented in the source documents and CRF. Deviations from the prescribed dosage regimen should be recorded in the eCRF. A record of the number of canisters dispensed to and returned by each subject (including the dispense/return dates, canister identification numbers, and returned canister weights) must be maintained and reconciled with study drug and compliance records.

### Concomitant Therapy

Any medication (including over-the-counter or prescription medicine, vitamin, and/or herbal supplement) or vaccine that the subject is receiving at the time of Screening or receives during the study must be recorded along with: Reason for use; dates of administration including start and end dates; dosage information including dose and frequency. Unless, in the opinion of the investigator and Sponsor, the concomitant medication will not interfere with the study, subjects must abstain from taking prescription or nonprescription drugs (including vitamins and dietary or herbal supplements) within 7 days (or 14 days if the drug is a potential enzyme inducer) or 5 half-lives (whichever is longer) before the start of the Pretreatment Visit and until completion of the EOS Follow-up Phone Call. The medical monitor should be contacted if there are any questions regarding concomitant or prior therapy. Protocol-specific rescue medications for bladder pain associated with IC/BPS and urinary tract infection; prohibited medications, medications requiring a washout period, and medications permitted under select circumstances; and protocol-permitted rescue medicines, are listed below All other concomitant medications may be considered on a case-by-case basis by the investigator in consultation with the medical monitor.

### Prohibited Medications, Medications Requiring a Washout Period, and Medications Permitted Under Select Circumstances

The following medications are prohibited from the Screening Visit until after the EOS Follow-up Phone Call: GC-C agonists (e.g., linaclotide, plecanatide); Diuretics (e.g., furosemide); Opioids; and Tricyclic antidepressants.

The following medications are prohibited from the Screening Visit until after the EOS Follow-up Phone Call and require a washout period: Intravesical therapy: 3-month washout period; Pentosan polysulfate (ELMIRON): 3-month washout period;

The following medications may be permitted under the specific circumstances outlined: Antidepressants (other than tricyclic antidepressants): permitted if prescribed for depression (i.e., not pain control) and the subject has been on a stable dose for >30 days prior to the Pretreatment Visit and plans to continue on the same dose until after the EOS Follow-up Phone Call. Antihistamines, antispasmodics, anticholinergics: permitted if subject has been on a stable dose for >30 days prior to the Pretreatment Visit and plans to continue on the same dose until after the EOS Follow-up Phone Call. Antihistamines for treatment of allergy symptom flares may be used but only for ≤2 days. Amphetamines: permitted if prescribed for a non-IC/BPS-related condition and the subject has been on a stable dose for >30 days prior to the Pretreatment Visit and plans to continue on the same dose until after the EOS Follow-up Phone Call. Amphetamines should be recorded as concomitant medications regardless of medical need. Cannabidiol (CBD): permitted if subject has been on a stable dose for >30 days prior to the Pretreatment Visit and plans to continue on the same dose until after the EOS Follow-up Phone Call. CBD should be recorded as concomitant medications regardless of medical need. Other pain medications (e.g., pregabalin, gabapentin, barbiturates): permitted for nonbladder pain if subject has been on a stable dose for >30 days prior to the Pretreatment Visit and plans to continue on the same dose until after the EOS Follow-up Phone Call. Phenazopyridine hydrochloride (e.g., PYRIDIUM): permitted during the time of a confirmed, active urinary tract infection only (refer below).

### Rescue Medicine

At the Pretreatment Visit, the study site will supply rescue medicine to subjects for bladder pain associated with IC/BPS or active, confirmed urinary tract infection (refer below). The following rescue medicines may be used: acetaminophen (up to 2 tablets [325 mg/tablet] every 4 to 6 hours up to 10 tablets/day); and ibuprofen (up to 2 tablets [200 mg/tablet] every 4 to 6 hours up to 10 tablets/day). Beginning at the Pretreatment Visit, the subject must record their use of rescue medicine in the daily eDiary (refer below). Throughout the Pretreatment and Treatment Periods, the eDiary will send automated alerts to the investigator if subjects have excessive use of rescue medicine. If a subject reports rescue medicine use >3 days per week for 2 consecutive weeks, the investigator must review the use of rescue medicine with the subject. The investigator, in consultation with the medical monitor, will decide whether the subject should remain in the study or be discontinued.

### Discontinuation of Study Intervention

It may be necessary for a subject to permanently discontinue study drug. A subject will be discontinued from the study in the event of: A confirmed pregnancy; an SAE where the investigator recommends discontinuation. Dosing of study drug for an individual subject may be temporarily discontinued as detailed below; if the subject is unable to resume dosing, they should follow the instructions for permanent discontinuation below.

If treatment is permanently discontinued, then the subject will return to the clinic to have the Week 12/EOT assessments performed. Refer to the SoA for data to be collected at the time of intervention discontinuation and follow-up, and for any further evaluations that need to be completed. If the subject discontinues treatment due to an AE, the subject will be followed until the AE resolves, stabilizes, or can be explained as being unrelated to study drug.

### Study Suspension or Termination

The sponsor may stop enrollment prematurely because of a low recruitment rate, a change in the sample size based on the IA, or the study meeting prespecified efficacy criteria based on the IA. The sponsor may permanently terminate the study, or a component of the study, at any time. Reasons for temporary suspension of dosing and/or study termination may include, but are not limited to: Death or treatment-emergent SAE considered to be related to the peptide of the present disclosure that may preclude further dosing, in the opinion of the sponsor; change in opinion of the institutional review boards (IRBs), a DMC recommendation based on the results of the IA(s). or a regulatory authority decision; and low recruitment rate. In the case of temporary suspension of dosing, dosing may be reinitiated or permanently halted after evaluation by the DMC.

### Temporary Discontinuation

The investigator may allow a subject to stop taking study drug for up to 3 days should an intolerable AE occur. If the investigator believes that the subject is unable to resume dosing or requires a suspension of dosing on more than 1 occasion, the investigator should contact the medical monitor to discuss the subject's continued participation in the study.

Subject Discontinuation/Withdrawal from the Study

A subject may withdraw from the study at any time at their own request or may be withdrawn at any time at the discretion of the investigator for safety, behavioral, compliance, or administrative reasons. This is expected to be uncommon. At the time of discontinuing from the study, if possible, an EOT visit should be conducted, as shown in the SoA. Refer to the SoA for data to be collected at the time of study discontinuation and follow-up and for any further evaluations that need to be completed. The subject will be permanently discontinued both from the study intervention and from the study at that time. If the subject withdraws consent for disclosure of future information, the sponsor may retain and continue to use any data collected before such a withdrawal of consent. If a subject withdraws from the study, they may request destruction of any samples taken and not tested, and the investigator must document this in the site study records.

### Lost to Follow-Up

A subject will be considered lost to follow-up if they repeatedly fail to return for scheduled visits and is unable to be contacted by the study site. The following actions must be taken if a subject fails to return to the clinic for a required study visit: The site representative must attempt to contact the subject and reschedule the missed visit as soon as possible and counsel the subject on the importance of maintaining the assigned visit schedule and ascertain whether or not the subject wishes to and/or should continue in the study.

Before a subject is deemed lost to follow-up, the investigator or designee must make every effort to regain contact with the subject (where possible, 3 telephone calls and, if necessary, a certified letter to the subject's last known mailing address or local equivalent methods). These contact attempts should be documented in the subject's medical record.

Should the subject continue to be unreachable, they will be considered to have withdrawn from the study and reason for discontinuation will be “lost to follow-up.”

### Example 30. Clinical Trial Assessments and Procedures

Study procedures and their timing are summarized in the SoA. Efficacy of the pharmaceutical rectal foam composition shall be determined based on the methods described below.

eDiary Assessments

Unless otherwise noted, eDiary assessments will be reported throughout the Pretreatment Period and Treatment Period. These consist of daily and weekly assessments.

Daily eDiary Assessments

### Daily Assessment of Bladder Pain Overall at its Worst

Subject assessment of bladder pain overall (which can include sensations like burning, pressure, and/or discomfort) at its worst will be reported via a daily evening report in the eDiary at baseline (Pretreatment Period) and throughout the Treatment Period. The rating of bladder pain overall (which can include sensations like burning, pressure, and/or discomfort) at its worst during the previous 24 hours on an 11-point NRS will be provided by the subject answering the following question: “How would you rate your worst bladder pain overall (which can include sensations like burning, pressure, and/or discomfort) in the past 24 hours?” eDiary presents NRS where 0 is anchored with “None” and 10 is anchored with “Worst possible”

### Assessment of a Burning Sensation in the Bladder at its Worst

Subject assessment of a burning sensation in the bladder at its worst will be reported via a daily evening report in the eDiary at baseline (Pretreatment Period) and for 7 days before the Week 4, Week 8, and Week 12 time points. The rating of a burning sensation in the bladder at its worst during the previous 24 hours on an 11-point NRS will be provided by the subject answering the following question: “How would you rate your worst burning sensation in the bladder in the past 24 hours?” eDiary presents NRS where 0 is anchored with “None” and 10 is anchored with “Worst possible”

### Assessment of a Pressure Sensation in the Bladder at its Worst

Subject assessment of a pressure sensation in the bladder at its worst will be reported via a daily evening report in the eDiary at baseline (Pretreatment Period) and for 7 days before the Week 4, Week 8, and Week 12 time points. The rating of a pressure sensation in the bladder at its worst during the previous 24 hours on an 11-point NRS will be provided by the subject answering the following question: “How would you rate your worst pressure sensation in the bladder in the past 24 hours?” eDiary presents NRS where 0 is anchored with “None” and 10 is anchored with “Worst possible”

### Assessment of Discomfort in the Bladder at its Worst

Subject assessment of discomfort in the bladder at its worst will be reported via a daily evening report in the eDiary at baseline (Pretreatment Period) and for 7 days before the Week 4, Week 8, and Week 12 time points. The rating of discomfort in the bladder at its worst during the previous 24 hours on an 11-point NRS will be provided by the subject answering the following question: “How would you rate your worst discomfort in the bladder in the past 24 hours?” eDiary presents NRS where 0 is anchored with “None” and 10 is anchored with “Worst possible”

### Urinary Frequency

Urinary frequency will be reported at baseline (Pretreatment Period) and for 7 days before the Week 4, Week 8, and Week 12 time points. The assessment of urinary frequency is based on the eDiary questions that record the date and time. Subjects will report each urination on an event-driven basis and indicate if the urination caused them to wake from sleep. If the subject misses an entry, they can enter the urination into the eDiary at a later time and include the actual date and time of the urination.

### Urinary Urgency

Urinary urgency will be reported at baseline (Pretreatment Period) and for 7 days before the Week 4, Week 8, and Week 12 time points. Subject assessment of urgency will be collected by the eDiary on an event-driven basis for each urination reported in the eDiary. If the subject misses a urination entry, they can enter the urination and associated assessment of urgency into the eDiary at a later time.
0=No urgency
1=Mild urgency
2=Moderate urgency
3=Severe urgency
4=Urge incontinence

### Difficulty Sleeping

Difficulty sleeping will be reported via a daily evening report in the eDiary at baseline (Pretreatment Period) and for 7 days before the Week 4, Week 8, and Week 12 time points. The ratings of difficulty sleeping will be provided by the subject answering the following 2 questions:

“How much difficulty did you have sleeping because of your bladder pain?”
1=None
2=Mild
3=Moderate
4=Severe
5=Very severe

“How much difficulty did you have sleeping because of a need to urinate?”
1=None
2=Mild
3=Moderate
4=Severe
5=Very severe

### Use of Rescue Medicine

Subjects will report their use of rescue medicine on a daily basis at baseline (Pretreatment Period) and throughout the Treatment Period by eDiary entry. An evening report will ask whether the subject used acetaminophen or ibuprofen and the number of tablets taken that day.

### Study Drug Administration

Subject will report their use of study drug on a daily basis throughout the Treatment Period by eDiary entry. The daily assessment will be provided by the subject answering the following question: “Did you administer the study drug today?”
1=Yes
2=No

If the answer is “1=Yes”, 2 additional questions will be asked.

### Canister-Related Dosing Problems

Subject assessment of canister-related dosing problems will be reported daily by eDiary entry throughout the Treatment Period. The daily assessment of canister-related dosing problems will be provided by the subject answering the following question: “Did you have any dosing problems related to the canister today?”
1=Yes
2=No

### Study Drug Retention

Subject assessment related to study drug retention will be reported daily by eDiary entry throughout the Treatment Period. The daily assessment of study drug retention will be provided by the subject answering the following question: “Did you have any foam leakage after taking study drug today?”
1=Yes
2=No
   Weekly eDiary Assessments

### General Weekly Questions

### Weekly Assessment of IC/BPS Symptom Severity

Subject global impression of IC/BPS severity will be reported weekly in the eDiary each week of the Pretreatment and Treatment Periods. The rating of overall IC/BPS severity during the previous 7 days on a 5-point ordinal scale will be provided by the subject answering the following question: “How would you rate your overall IC/BPS symptoms during the past 7 days?”
1=None
2=Mild
3=Moderate
4=Severe
5=Very severe

### Weekly Assessment of Treatment Satisfaction

Subject assessment of treatment satisfaction will be reported weekly by eDiary entry each week of the Treatment Period. Subjects will answer the following question on a 5-point ordinal scale: “Overall, how satisfied are you with the study drug's ability to relieve your IC/BPS pain during the past 7 days?”
1=Not at all satisfied
2=A little satisfied
3=Moderately satisfied
4=Quite satisfied
5=Very satisfied

### Weekly Assessment of Adequate Relief

Subject assessment of adequate relief of IC/BPS pain will be reported by an eDiary entry each week of the Treatment Period. The rating of adequate relief during the previous 7 days on a binary scale will be provided by the subject answering the following question: “Overall, have you had adequate relief from your bladder pain during the past 7 days?”
1=Yes
2=No

### Weekly Assessment of IC/BPS Symptom Change

Subject global impression of IC/BPS severity will be reported weekly in the eDiary each week of the Treatment Period. The rating of overall IC/BPS severity during the previous 7 days on a 7-point ordinal scale will be provided by the subject answering the following question: “Compared to before you started the study, please rate your overall change in IC/BPS symptoms during the past 7 days.”
1=Very much better
2=Moderately better
3=A little better
4=No change
5=A little worse
6=Moderately worse
7=Very much worse

### Weekly Assessment of Sexual Function

The sexual function question will be reported weekly in the eDiary each week of the Treatment Period. The answer on a 7-point ordinal scale will be provided by the subject answering the following question: “Compared to before you started the study treatment, how would you rate your ability to have sexual intercourse?”
−3=Markedly worse
−2=moderately worse
−1=slightly worse
0=no change
1=slightly better
2=moderately better
3=markedly better
99=not applicable, prefer not to answer

### Genitourinary Pain Index (GUPI)

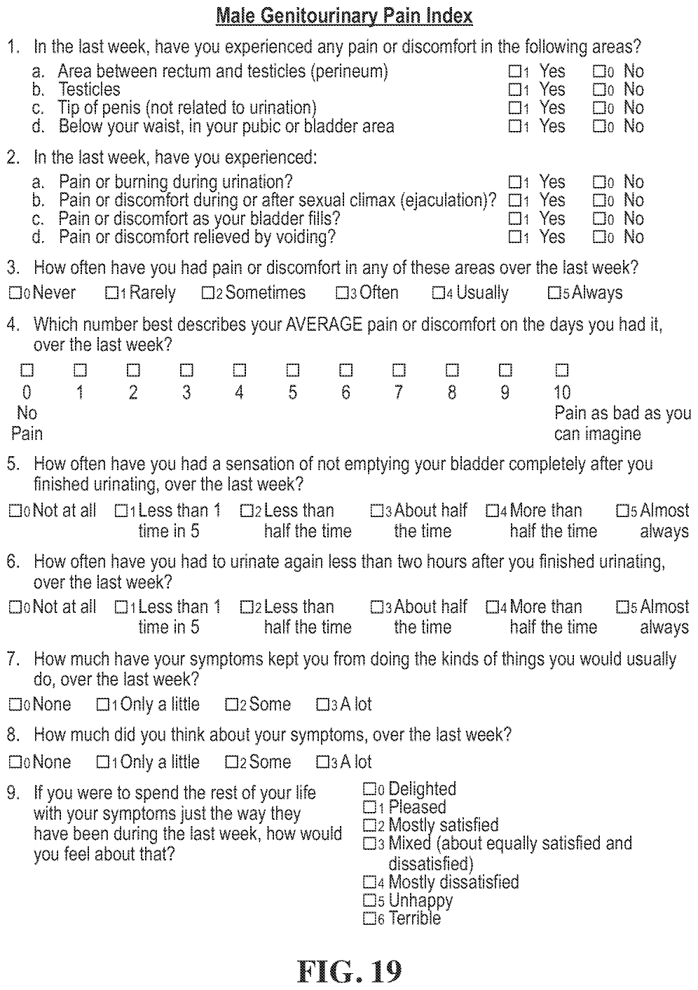

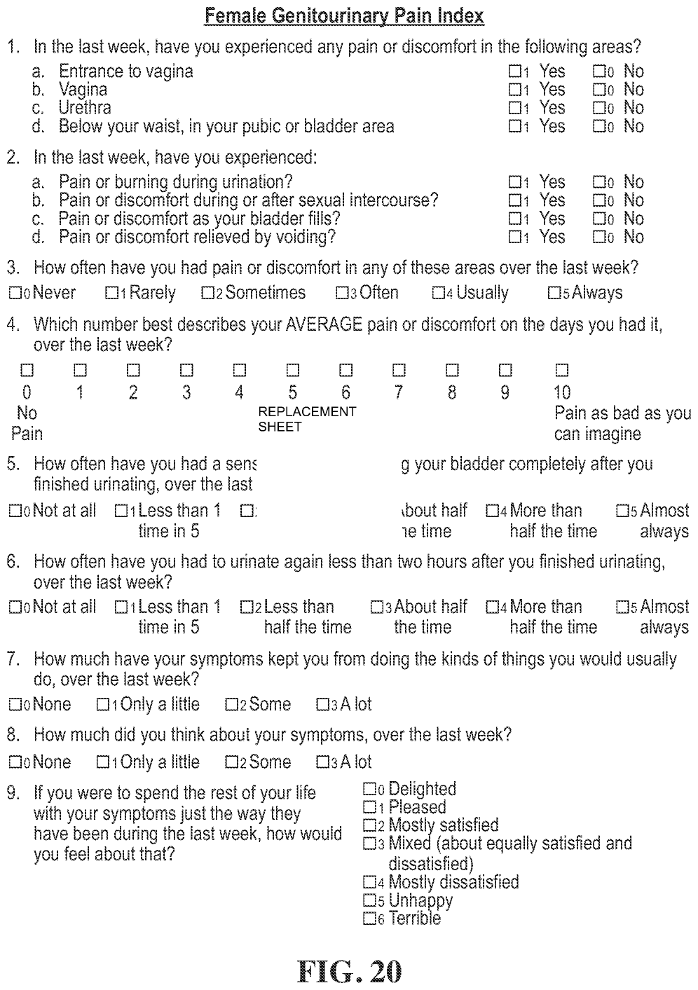

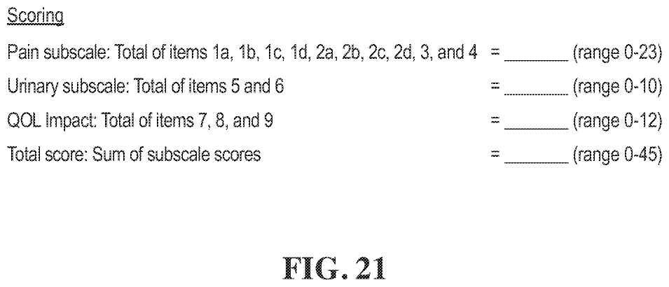

The GUPI assesses the degree of symptoms in both men and women with genitourinary pain complaints over the last week, including experience (Yes/No) of pain or discomfort in various areas, frequency of pain/discomfort (0=Never to 5=Always), average pain/discomfort (11-point NRS), frequency of urinary symptoms (0=Not at all to 5=Almost always), and quality of life (QoL) impact (0=None to 3=A lot; or 1=Pleased to 6=Terrible). FIGS. 19-21.

### Global Response Assessment (GRA)

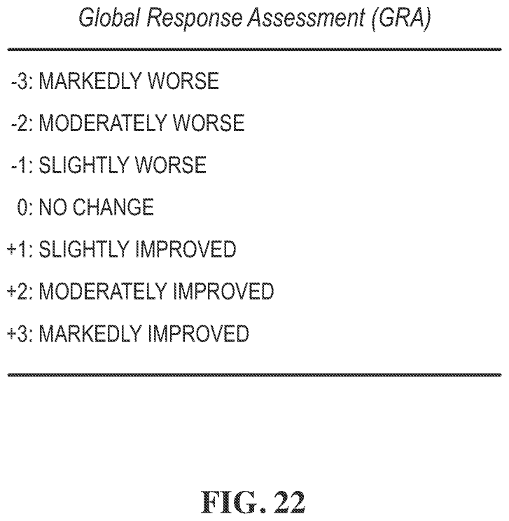

The GRA measures overall improvement with therapy, asking “As compared to when you started the study [treatment], how would you rate your interstitial cystitis symptoms now?” The response is recorded on a 7-point scale centered at zero (no change): markedly worse (=−3); moderately worse (=−2); slightly worse (=−1); no change (=0); slightly improved (=+1); moderately improved (=+2); and markedly improved (=+3). FIG. 22 shows the GRA questionnaire.

Monthly eDiary Assessment

### O'Leary/Sant Interstitial Cystitis Symptom Index (ICSI)

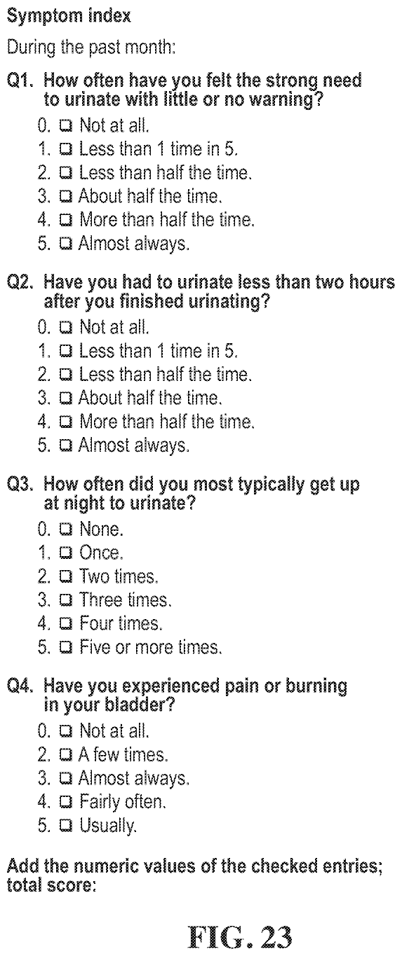

The O'Leary/Sant ICSI assesses the incidence of IC symptoms during the past month. Questions on urgency and frequency are scored on a scales of either ‘0=Not at all to 5=Almost always’. The question on nocturia is scored on a scale of ‘0=None to 5=5 or more times’. The question on pain or burning in the bladder is scored on a scale of ‘0=Not at all to 5=Usually’. FIG. 23 shows the O'Leary/Sant Interstitial Cystitis Symptom Index (ICSI) questionnaire.

### Margolis Body Map for Pain Assessment

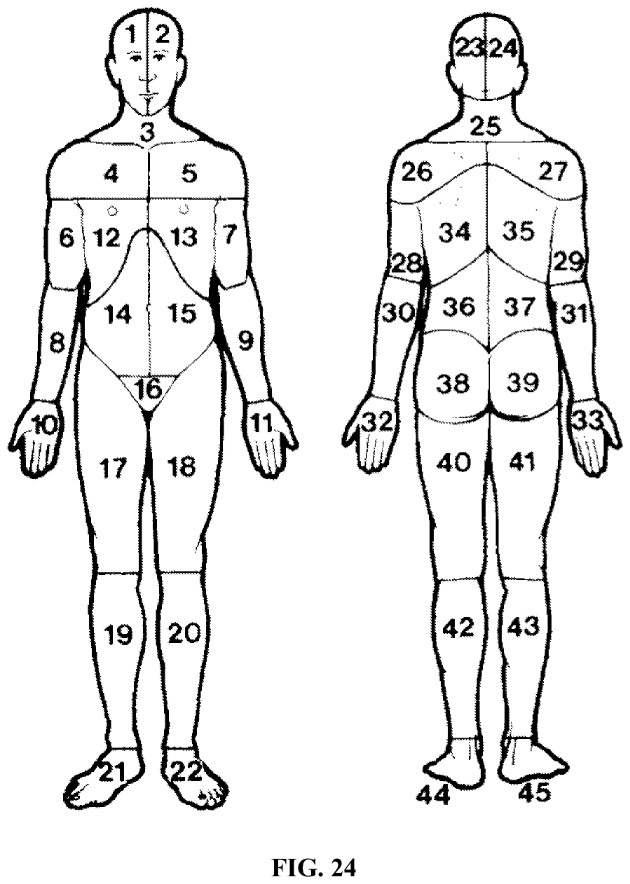

At the study visits shown in the SoA, pain location will be assessed using the Margolis body map. See Margolis R B, Tait R C, Krause S J. A rating system for use with patient pain drawings. Pain. 1986; 24:57; the disclosure of which is incorporated herein by reference in its entirety. Briefly, subjects will be asked to check any of 45 body sites on the Margolis body map where they experienced pain in the past week. FIG. 24 shows the Margolis Body Map for Pain Assessment questionnaire. Subjects reporting pain in Sites 14, 15 or 16 only will be considered to have “pelvic pain only,” a designation used by Nickel et al in a previous pain pattern study in women with IC/BPS. See Nickel J C, Tripp D A. International Interstitial Cystitis Study G. Clinical and psychological parameters associated with pain pattern phenotypes in women with interstitial cystitis/bladder pain syndrome. J Urol. 2015; 193:138; the disclosure of which is incorporated herein by reference in its entirety.

Using the subject's completed body maps and a classification system described by Lai et al; MAPP Research Network. Characterization of Whole Body Pain in Urological Chronic Pelvic Pain Syndrome at Baseline: A MAPP Research Network Study. J Urol. 2017 September; 198(3):622-631 (the disclosure of which is incorporated herein by reference in its entirety), the subject's body pain will be categorized in as:
Pelvic pain only.
Pelvic pain and 1 to 2 other sites.
Pelvic pain and 3 to 7 other sites.
Pelvic pain and 7+ other sites

### Safety Assessments

The planned time points for all safety assessments are provided in the SoA.

### Physical Examinations

Physical examinations will be performed as outlined in the SoA, by the investigator or a licensed health professional listed on Form FDA 1572. A complete physical examination will include, at a minimum, assessments of the general appearance of the subject and the HEENT (head, eyes, ears, nose, and throat), cardiac, respiratory, GI, musculoskeletal, neurological, and dermatological systems. A visual and digital rectal examination will include a visual inspection of the perianal area for redness or irritation, and will be performed at Screening and Week 12/EOT. A visual rectal examination will be performed at other visits. If significant redness, rectal pain, bleeding, or mucus is observed on visual inspection of the rectal (perianal) area, a digital rectal examination and hemoccult test should be performed. Additionally, anoscopy may be performed as part of the rectal examination. If further investigation is warranted, subjects with these signs or symptoms should undergo proctoscopy or recto-sigmoidoscopy. If these tests do not identify the source of inflammation, a colonoscopy may be performed after a discussion between the investigator and the sponsor.

Height (only at Screening) and weight will also be measured and recorded. Investigators should pay special attention to clinical signs related to previous serious illnesses, which will be reason for exclusion from the study. Any physical examination abnormality that the investigator considers to be potentially clinically significant and changed from baseline will be reported as an AE.

### Vital Signs

Vital signs will be performed as outlined in the SoA. Oral temperature, respiratory rate, blood pressure, and pulse rate will be assessed. Blood pressure and pulse measurements will be taken in the seated position with a completely automated device. Manual techniques will be used only if an automated device is not available. Blood pressure and pulse measurements will be taken after the subject has been sitting for 5 minutes in a quiet setting without distractions (e.g., television, cell phones). When applicable, vital signs measurements will be obtained prior to blood draws.

### Electrocardiograms

Triplicate 12-lead electrocardiogram (ECG) will be obtained as outlined in the SoA using an ECG machine that automatically calculates the heart rate and measures PR, QRS, QT, and QTcF intervals. ECGs should be obtained after the subject has been supine for at least 5 minutes and prior to blood draws, when applicable. At each time point at which triplicate ECGs are required, 3 individual ECG tracings should be obtained as closely as possible in succession, but no more than 2 minutes apart.

### Pregnancy Testing

Inclusion Criteria, for screening pregnancy testing criteria is described below. Pregnancy testing must be conducted at the time points indicated in the SoA. Pregnancy testing (urine or serum as required by local regulations) should be conducted at the end of relevant systemic exposure. Additional serum or urine pregnancy tests may be performed, as determined necessary by the investigator or required by local regulation, to establish the absence of pregnancy at any time during the subject's participation in the study.

### Medical History

In addition to general medical history, baseline data on a subject's IC/BPS characteristics and prior symptom management will also be collected at the Screening Visit.

For IC/BPS characteristics, the following data will be collected: date of first diagnosis (month, year); findings from prior cystoscopy, if available (e.g., date of prior cystoscopy (day/month/year); presence of Hunner's lesions [yes/no/unknown], presence of glomerulations [yes/no/unknown]); and presence of associated conditions (e.g., IBS, fibromyalgia, chronic fatigue syndrome, anxiety and depression disorders, migraines). For prior IC/BPS symptom management, the questionnaire (see below) will be administered by the site staff, incorporated within the subject's source documentation, and entered into the eCRF.

TABLE 78
Prior IC/BPS symptom management assessment.

| | Questions | | | | | |
|---|---|---|---|---|---|---|
| | Has the | | | | | |
| | subject ever | | If yes, when was the | | If the subject stopped | |
| | used this for | | last time it was | | this, what was the | |
| | IC/BPS? | | taken/used? | | primary reason? | |
| | Responses | | | | | |
| | | | | | | Not |
| | | | | | | applicable, |
| | | | | Currently using (record | | currently |
| | | | | in the concomitant meds | | taking |
| | | | | eCRF) | | Did not improve |
| | | | | Less than 6 months ago | 1. | symptoms |
| | | | 1. | More than 6 months | 2. | Experienced side |
| | | | 2. | ago, but less than 1 | 3. | effects |
| Therapeutic | 1. | Yes | 3. | year ago | 4. | Costs too much |
| modality↓ | 2. | No | 4. | Over 1 year ago | 5. | Other |

Oral medications
Intravesical medications
Cystoscopy
Hydrodistension
Physical therapy
Electrical stimulation
Homeopathic/naturopathic
Diet modifications
Lifestyle modifications
Cognitive behavioral therapy/
Psychotherapy
Surgery
Other:
<<List modality>>

### Suicidal Ideation and Behavior Risk Monitoring

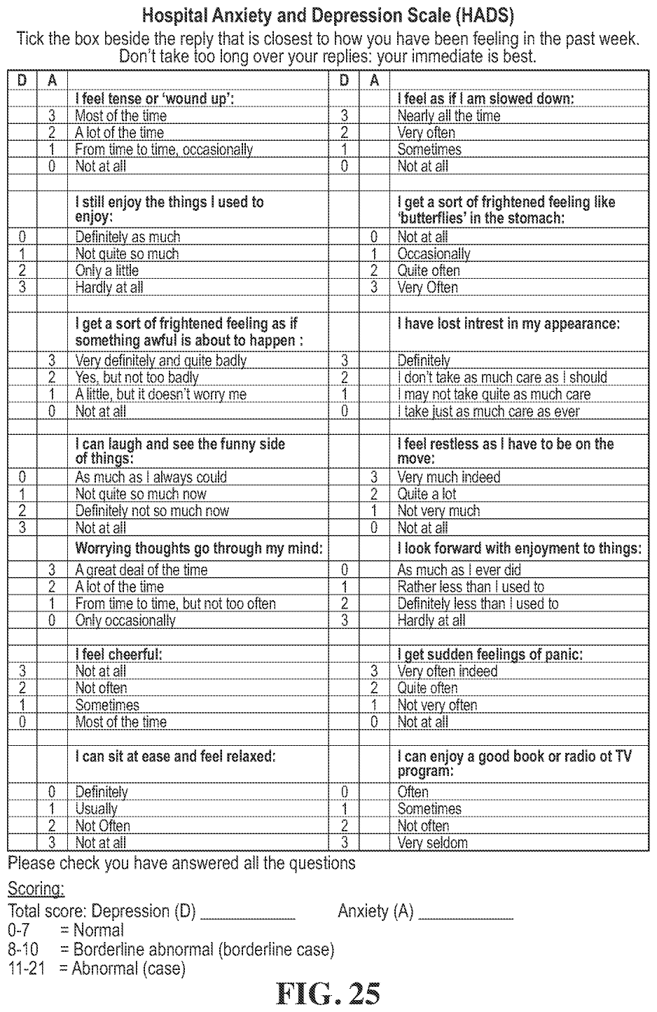

Patients with IC/BPS have an increased risk for depression and may be at increased risk for suicide. (12) Anxiety and depression will be assessed using the Hospital Anxiety and Depression Scale (HADS) (FIG. 25) at the time points shown in the SoA. Subjects who have an uncontrolled major psychiatric condition or have made a suicide attempt during the 2 years before the Screening Visit are excluded from the study. The peptide of the present disclosure is not considered to be a central nervous system-active drug, nor is it related to products with an increased risk of suicidal ideation or behavior. However, consideration should be given to discontinuing the study drug in subjects who experience signs of suicidal ideation or behavior.

### Adverse Events and Serious Adverse Events

AEs will be reported by the subject (or, when appropriate, by a caregiver, surrogate, or the subject's legally authorized representative). The investigator and any qualified designees are responsible for detecting, documenting, and recording events that meet the definition of an AE or SAE and remain responsible for following up on AEs that are serious, considered related to the study intervention or study procedures, or that caused the subject to discontinue study drug.

### Pregnancy

Details of all pregnancies that occur in female subjects and female partners of male subjects following study drug exposure will be collected either until completion of the EOS Follow-up Phone Call or until the details regarding the pregnancy outcome are reported. The investigator should make a reasonable effort to follow any pregnant patients until delivery or end of the pregnancy. If a pregnancy is reported, the investigator should inform the sponsor within 24 hours of learning of the pregnancy and should follow the procedures outlined below. Abnormal pregnancy outcomes (e.g., spontaneous abortion, fetal death, stillbirth, congenital anomalies, ectopic pregnancy) are considered SAEs.

### Cardiovascular and Death Events

Cardiovascular and death events should be reported according to the definitions and reporting procedures for AEs and SAEs as known in the art.

### Urinary Tract Infections

Urinary tract infections must be reported according to the definitions and reporting procedures for AEs and SAEs as known in the art. Additional clinical data on urinary tract infections will be collected. Urinary tract infections must be confirmed by a urinalysis and urine culture.

### Treatment of Overdose

For this study, any dose that exceeds the dosage or frequency specified in the protocol will be considered an overdose. The sponsor does not recommend specific treatment for an overdose. In the event of an overdose, the investigator should: Report any case of overdose to the sponsor and use their clinical judgment to treat the case of overdose as needed with the appropriate general supportive measures; closely monitor the subject for any AE/SAE and laboratory abnormalities until study drug can no longer be detected systemically; and document the quantity of the excess dose as well as the duration of the overdose in the eCRF. Decisions regarding dose interruptions will be made by the investigator in consultation with the Medical Monitor based on the clinical evaluation of the subject.

### Pharmacokinetics (PK)

Plasma will be collected for measurement of plasma concentrations of the peptide of the present disclosure as specified in the SoA. Instructions for the collection and handling of biological samples will be provided by the sponsor. The actual date and time (24-hour clock time) of each sample will be recorded. Samples will be used to evaluate the PK of the peptide of the present disclosure. Each plasma sample will be divided into 2 aliquots. PK samples may be analyzed for drug concentrations in batches on a periodic basis as the study is being conducted. However, information that may unblind the study will not be reported to investigative sites or other blinded personnel at the site, sponsor, or CRO until after the study has been unblinded.

Health-related Quality of Life

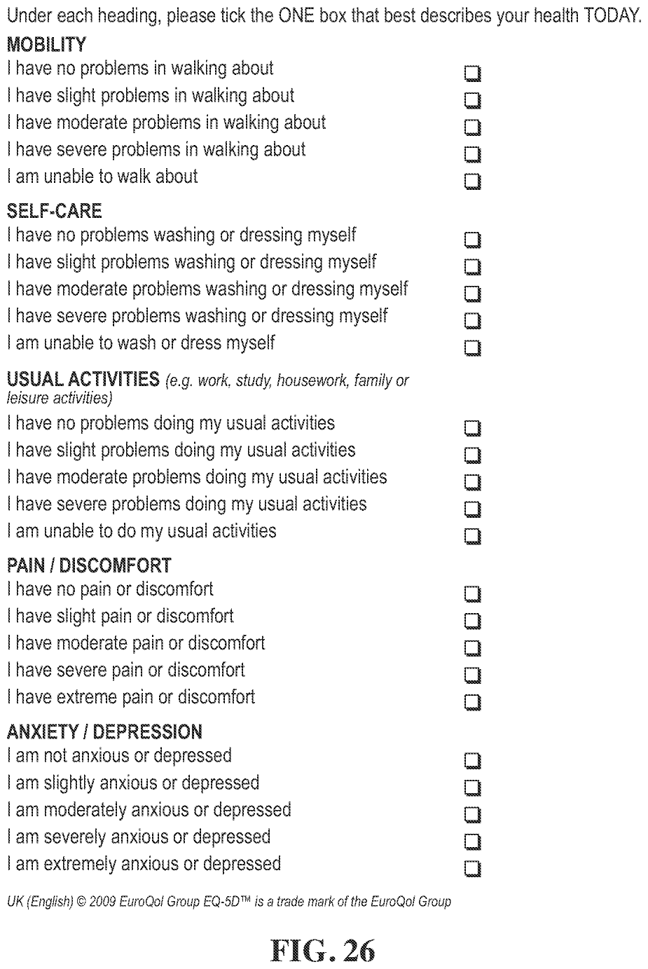

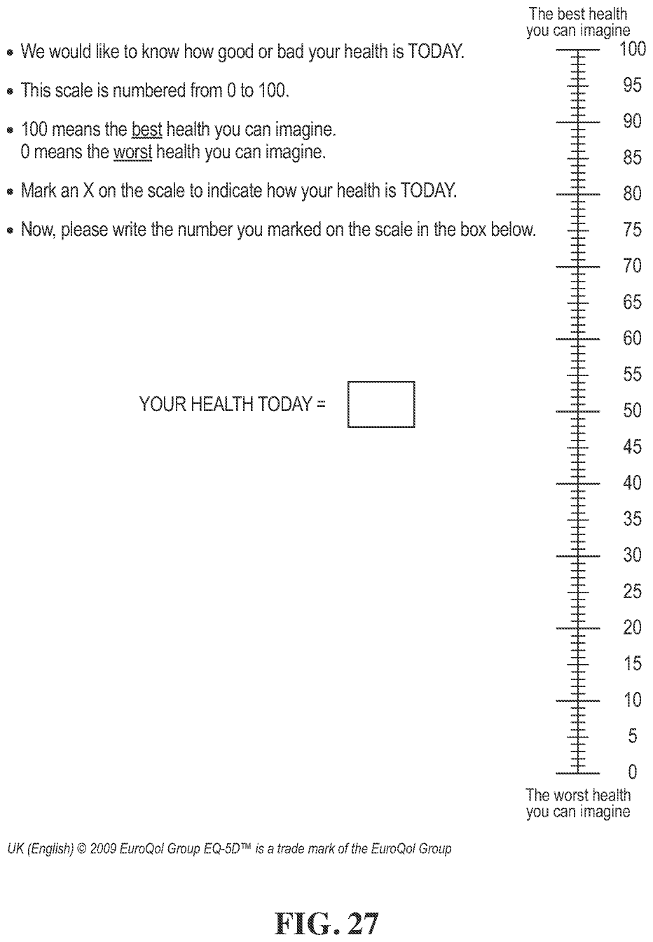

The EuroQol 5-Dimension 5-Level Version (EQ-5D-5L) questionnaire will be captured at the time points noted in the SoA. FIG. 26 shows the EuroQoL Group EQ-5D questionnaire. FIG. 27 shows the EuroQoL Group EQ-5D health scale questionnaire.

### Microbiome

Gut microbiome will be assessed as an exploratory evaluation in a subset of approximately 60 subjects who complete the study (˜20/arm) using stool samples collected in accordance with the SoA. At the visits required, subjects in the microbiome substudy will bring in a stool sample using the sample collection kit issued by the site. The methods for collecting, handling, and processing samples will be provided in a laboratory manual.

### Example 31. Clinical Study Statistical Considerations

### Statistical Hypotheses

The primary efficacy endpoint of the treatment differences for CFB in bladder pain at its worst at Week 12 will be assessed to test the following hypothesis:

$${H}_{0} : {μ}_{100 µ g} - {μ}_{P l a c e b o} \geq 0 and {μ}_{300 µ g} - {μ}_{P l a c e b o} \geq 0 ,$$

$${H}_{a} : {μ}_{100 µ g} - {μ}_{P l a c e b o} \geq 0 or {μ}_{300 µ g} - {μ}_{P l a c e b o} < 0.$$

Each of the active dose strength arms (100 μg or 300 μg) will be compared with the placebo arm at a 1-sided 0.025 significance level. This multiplicity adjustment for the primary efficacy endpoint will control the a level at 1-sided significance level of 0.05.

### Sample Size Determination

The sample size and power for the study was estimated based on simulations with Bayesian hierarchical models for the primary efficacy endpoint. The assumption of the placebo effect was obtained by augmenting the historical information of effects of placebo treatment from 4 publications of randomized placebo-controlled studies in subjects with IC/BPS, which utilized the same bladder pain endpoint as measured on an 11-point NRS and similar treatment durations; as well as the effects of placebo treatment from 3 previous studies conducted with linaclotide (another GC-C agonist with similar activity and same mechanism of action) in subjects with IBS-C, which utilized a similar abdominal pain endpoint that was also measured on an 11-point NRS. The effect of the active treatment was obtained through meta-analysis of these 3 previous studies using linaclotide in subjects with IBS-C. The overall study-wise type I error of 0.10 is equally split between the 2 comparisons of the primary efficacy endpoint among the peptide of the present disclosure doses relative to placebo following Bonferroni correction; an alpha of 0.05 will be spent for each comparison. Based on the simulations, a sample size of at least 85 subjects per arm will yield 81% power to confirm superiority at the 0.05 alpha level. With a 10% to 15% attrition rate, approximately 95 to 100 subjects per arm will need to be randomized.

This sample size represents the maximum number of subjects to be enrolled based on initial assumptions. The study may be stopped early for efficacy, a treatment arm may be discontinued if it does not warrant further exploration, or enrollment may be stopped early for futility. Given these scenarios, the number of subjects planned for enrollment could change.

### Populations for Analyses

TABLE 79
The following analysis populations are defined:

| Analysis Set | Description |
|---|---|
| Screened Analysis Set | The Screened Analysis Set consists of all subjects who have signed |
| | informed consent. |
| Intent-to-Treat (ITT) | The ITT Analysis Set consists of all subjects who are randomized to |
| Analysis Set | a treatment regimen. Analysis will be performed according to the |
| | treatment assigned at the Day 1 Visit. |
| Safety Analysis Set | The Safety Analysis Set consists of all subjects who receive any |
| | amount of study drug. Analysis will be performed according to the |
| | treatment actually received regardless of the allocated treatment. |
| PK Analysis Set | The PK Analysis Set consists of all subjects who receive any |
| | amount of study drug and have at least 1 postdose PK assessment. |
| | Analysis will be performed according to treatment actually received |
| | regardless of the treatment regimen allocated. |
| Microbiome Analysis Set | The Microbiome Analysis Set consists of all subjects in the Safety |
| | Analysis Set who have at least 1 postdose microbiome assessment. |
| | Analysis will be performed according to the treatment actually |
| | received regardless of the allocated treatment. |

The statistical analysis plan (SAP) will be finalized prior to database lock. The SAP will include a more technical and detailed description of the statistical analyses. A separate IA plan will be written to cover the details of the interim analyses.

In general, descriptive statistics will be presented by treatment group and by visit, as applicable. For analysis of continuous parameters (e.g., change from baseline), descriptive statistics (n, mean, standard deviation [SD], median, and range) will be calculated and presented for each visit and treatment. For categorical parameters (e.g., responder vs. nonresponder), the number and percentage of each category will be calculated and presented for each visit and treatment. Baseline values for the efficacy parameters will be generated based on from the eDiary data collected in the last 14 days of the Pretreatment Period. Further details will be available in the SAP. The 2 comparisons of the primary efficacy endpoint among the peptide of the present disclosure doses relative to placebo will be adjusted using Bonferroni correction, i.e., the type I error of 0.10 will be equally split between the 2 comparisons; an alpha of 0.05 will be spent for each comparison. Multiple comparisons will not be adjusted in the conduct of comparisons among the peptide of the present disclosure doses relative to placebo for all other efficacy endpoints. Missing data will be imputed for efficacy analysis. Statistical analyses will be conducted using SAS version 9.4 (or later), and a statistical package that was developed by Berry Consultants.

### Primary Endpoint Analysis

All efficacy analysis will be performed on the ITT Analysis Set. The primary efficacy endpoint is the change from baseline in weekly average of daily bladder pain at its worst at Week 12. A frequentist 2-sample t-test will be performed to test the hypotheses; 1-sided 97.5% CIs and the corresponding p-values will be provided for the treatment differences for the change from baseline in bladder pain at week 12 between 100 μg vs. Placebo and 300 μg vs. Placebo. As a sensitivity analysis, a Bayesian hierarchical model will be implemented to confirm the results from the frequentist approaches.

### Secondary Endpoint Analysis

The secondary efficacy endpoints analyses will be conducted on the ITT population. Statistical comparisons of placebo and active arms on the change from baseline in weekly average of a burning sensation, pressure sensation, or discomfort in the bladder at its worst at Week 12 will be conducted, respectively. A similar analysis will be performed for change from baseline in GUPI Pain subscale score. The secondary safety endpoint analyses will be performed on the Safety Analysis Set. The number and percentage of subjects reporting TEAEs with an occurrence of ≥2% (in any peptide of the present disclosure dose strength) of subjects in each treatment group will be tabulated by MedDRA system organ class and preferred term. If a subject has >1 TEAE coded to the same preferred term, the subject will be counted only once for that preferred term. Statistical tests will not be performed. The overall number and percentage of subjects reporting TEAEs in each treatment group will be tabulated by severity grade.

### Exploratory Endpoints Analyses

The appropriate analysis set will be applied for each of the exploratory endpoints. Statistical comparisons will not be performed as these endpoints are exploratory in nature; a summary of descriptive statistics will be presented.

### Safety Analyses

All safety analyses will be performed on the Safety Analysis Set. All safety parameters will be summarized using descriptive statistics. For each safety parameter, the last nonmissing assessment made before the first dose of study drug will be used as the baseline for all analyses of that safety parameter.

### Pharmacokinetics

The PK analysis will be performed on the PK Analysis Set. If systemic levels of the peptide of the present disclosure are measurable, plasma concentrations of the peptide of the present disclosure will be summarized using appropriate descriptive statistics by the peptide of the present disclosure dose strength and overall at each assessment time point.

### Microbiome

The analysis of the microbiome will be performed on the Microbiome Analysis Set. This analysis will be conducted in a subgroup of subjects, comprising approximately 60 subjects who complete the study (˜20/arm). The relative abundance of different family, genus, and species will be constructed and summarized by treatment at Baseline and Week 12/EOT using descriptive statistics. Changes from baseline will be calculated.

### Health-Related Quality of Life

The EQ-5D-5L utility scores and Visual Analog Scale scores will be summarized for each visit using descriptive statistics by treatment for the Safety Analysis Set.

### Interim Analyses

At least one interim analyses (IA) will be performed during the study to provide an early assessment of the efficacy or futility of the peptide of the present disclosure. The purpose of the IA(s) is to stop the study early for efficacy if there is overwhelming evidence of effectiveness, to discontinue a treatment arm if it does not warrant further exploration, or to stop the enrollment early for futility. In some embodiments, the first IA will be conducted when approximately 30% of subjects have completed 8 weeks on treatment. If more than 1 IA is conducted, the subsequent IA(s) will be equally spaced.

The 1-sided, study-wise type I error will be controlled at 0.05 level for the primary efficacy analysis. Each of the 2 superiority comparisons of the dose strengths (peptide of the present disclosure having a dose of about 100 μg and about 300 μg) will be controlled at 1-sided 0.025 level against the placebo arm. The actual alpha-spending level will be determined by the Kim-Demets alpha spending function.

Regardless of whether the decision is to stop the study early or to continue to the end of the study, formal statistical comparisons will be conducted at the end of the study based on the prespecified alpha-spending level.

### Example 32. Clinical Laboratory Tests

All protocol-required laboratory assessments, as defined below, must be conducted in accordance with the Laboratory Manual and the SoA. The laboratory tests are detailed in the table below.

TABLE 80
Protocol-required Safety Laboratory Assessments

| Laboratory | | | | |
|---|---|---|---|---|
| Assessments | Parameters | | | |
| Hematology | Platelet count | RBC indices: | | WBC count with |
| | RBC count | Mean corpuscular volume | | differential: |
| | Hemoglobin | (MCV) | | Neutrophils |
| | Hematocrit | Mean corpuscular hemoglobin | | Lymphocytes |
| | | (MCH) | | Monocytes |
| | | Mean corpuscular hemoglobin | | Eosinophils |
| | | concentration (MCHC) | | Basophils |
| | | % Reticulocytes | | |
| Clinical | Blood urea | Potassium | Aspartate | Total and direct |
| Chemistry^{a} | nitrogen (BUN) | | aminotransferase | bilirubin |
| | | | (AST) | |
| | Creatinine | Sodium | Alanine | Total protein |
| | | | aminotransferase | |
| | | | (ALT) | |
| | Glucose | Calcium | Alkaline | Magnesium |
| | (random) | | phosphatase | |
| | Chloride | Albumin | Bicarbonate | Phosphate |
| | Cholesterol | Uric acid | | |

| | |
|---|---|
| Inflammatory | Erythrocyte sedimentation rate (ESR) |
| Markers | C-reactive protein (CRP) |
| Hemoccult | Point-of-care hemoccult test (In addition to the timepoints listed in the SoA, if |
| | significant redness, rectal pain, bleeding, or mucus is observed on visual |
| | inspection of the rectal [perianal] area, a digital rectal examination and |
| | hemoccult test should be performed. Stool that is present on the digital rectal |
| | exam can be used for the hemoccult test |
| Routine | Specific gravity |
| Urinalysis | pH, glucose, protein, blood, ketones |
| | Microscopic examination (if blood or protein is abnormal) |
| | Urine culture (performed if urinary tract infection is suspected) |
| Other Screening | Alcohol and drug screen (to include at minimum: amphetamines, barbiturates, |
| Tests | cocaine, opiates, cannabinoids, and benzodiazepines) |
| | HIV antibody and hepatitis panel (hepatitis B surface antigen [HBsAg] and |
| | hepatitis C virus [HCV] antibody) |
| Hormone tests for | Highly sensitive pregnancy test (WOCBP only) |
| female subjects | Follicle-stimulating hormone (FSH) and estradiol (WONCBP) |

All events of ALT >3 × upper limit of normal (ULN) and bilirubin >2 × ULN (>35% direct bilirubin) or ALT >3 × ULN and international normalized ratio (INR) >1.5, if INR is measured which may indicate severe liver injury (possible Hy's Law case), must be reported as an SAE (excluding studies of hepatic impairment or cirrhosis).

All protocol-required laboratory assessments, as defined below, must be conducted in accordance with the Laboratory Manual and the SoA.

### Example 33. Compounding Batch Observations

The present example concerns the production of two 26.0 kg pharmaceutical rectal foam composition placebo batches. The following is a summary of the 26.0 kg batch Process Description, observations, and testing results for pharmaceutical rectal foam composition placebo Lots SEZ-F and SFT-F. Table 78 below provides the formula composition and the amounts for the materials used.

TABLE 81
Components of pharmaceutical rectal foam composition placebo
Lots SEZ-F and SFT-F. Pharmaceutical rectal foam placebo

| | Formula Code | | | | |
|---|---|---|---|---|---|
| | PDGW-1 | SEZ-F | | SFT-F | |
| Ingredient | % w/w | Amount | | Amount | |
| Purified Water USP/EP | 77.550 | 20.2 | kg | 20.2 | kg |
| Propylene Glycol USP | 10.000 | 2.60 | kg | 2.60 | kg |
| Sodium Phosphate Dibasic USP | 0.164 | 42.6 | g | 42.6 | g |
| Sodium Dihydrogen Phosphate Monohydrate USP | 0.117 | 30.3 | g | 30.3 | g |
| Disodium EDTA USP | 0.050 | 13.0 | g | 13.0 | g |
| Methyl paraben NF | 0.100 | 26.0 | g | 26.0 | g |
| Light Mineral Oil NF | 6.000 | 1560 | g | 1560 | g |
| Isopropyl Myristate NF | 0.500 | 130 | g | 130 | g |
| White Petrolatum USP | 1.000 | 260 | g | 260 | g |
| Polyoxyl 20 Cetostearyl Ether NF | 2.500 | 650 | g | 650 | g |
| Cetyl Alcohol NF | 1.000 | 260 | g | 260 | g |
| Stearyl Alcohol NF | 1.000 | 260 | g | 260 | g |
| Propylparaben NF | 0.020 | 5.20 | g | 5.20 | g |
| Total | 100.00 | 26.0 | kg | 26.0 | kg |
| AP35 Propellant | 9.9 | 3.40 | g | 3.40 | g |

### Process Description: 26.0 kg Pharmaceutical Rectal Foam Placebo, Lot SEZ-F

### Primary Compounding Phase

The Primary Compounding Phase was prepared in a round bottom, jacketed, stainless steel Kettle (TK4896) (Capacity: 70 L) into which Purified Water USP/EP (19.2 kg) and Propylene Glycol USP (2.60 kg) were added. This was placed under a Cowles Dissolver (DS4879) with a 6-inch standard disperser blade. Mixing began at 355 RPM while Sodium Phosphate Dibasic USP (42.6 g), Sodium Dihydrogen Phosphate Monohydrate USP (30.3 g) and Disodium EDTA USP (13.0 g) were added. Mixing continued at 355 RPM for 10 minutes. Mixing was discontinued and the phase was observed to be a clear, colorless solution with no undissolved particles present. Using a stainless-steel container, 7.3 kg was transferred into a round bottom, jacketed stainless steel Kettle (TK4841) (Capacity: 10 L) for the side phase below.

Mixing resumed with the remaining primary Compounding phase at 325 RPM while heating with steam in the kettle jacket to 78° C. (Range: 75-80° C.). Methylparaben NF (26.0 g) was added and mixing continued at 325 RPM while for 46 minutes and a final temperature 77° C. (Range: 75-80° C.). Mixing was discontinued and the phase was observed to be a clear, colorless solution with no undissolved particles present.

### Oil Phase

The Oil Phase was prepared in a round bottom, jacketed, stainless steel Kettle (TK4902) (Capacity: 10 L) into which Mineral Oil NF (1560 g), Isopropyl Myristate NF (130 g), White Petrolatum USP (260 g), Polyoxyl 20 Cetostearyl Ether NF (650 g), Cetyl Alcohol NF (260 g), Stearyl Alcohol NF (260 g) and Propylparaben NF (5.20 g) were added. This was placed under a 1.5 HP Cowles Dissolver (DS4855) with a 3-inch standard disperser blade. Mixing began at 413 RPM while heating with steam in the kettle jacket to 76° C. (Range: 75-80° C.) which took 14 minutes. Mixing continued at 413 RPM for 10 minutes and a final temperature 78° C. (Range: 75-80° C.). Mixing was discontinued and the phase was observed to be a clear, colorless solution with no undissolved particles present.

### Emulsification Phase

The Oil Phase was added to the Primary Compounding Phase above with mixing at 325 RPM. This was followed with a hot Purified Water USP/EP rinse of the Oil Phase Kettle. Mixing continued at 375 RPM for 10 minutes while maintaining at 75-80° C., final temperature was 77° C. (Range: 75-80° C.). Mixing continued with ambient cooling water in the jacket at 375 RPM while cooling to 38° C. (Range: 40° C. or below) which took 15 minutes. Mixing continued at 375 RPM while cooling to 30° C. (Range: 30° C. or below) which took 15 minutes. The phase was observed to be a white to off-white thin liquid emulsion.

### Side Phase

The Side Phase was prepared in a round bottom, jacketed, stainless steel Kettle (TK4841) (Capacity: 10 L) into which 7.3 kg of the Primary Compounding Phase was added. This was placed under a 1.5 HP Cowles Dissolver (DS4855) with a 3-inch standard disperser blade. Mixing began at 495 RPM while cooling first with ambient and then chilled cooling water in the kettle jacket to 24° C. (Range: 20-25° C.) which took 28 minutes. Mixing was discontinued and the phase was observed to be a clear, colorless solution with no undissolved particles present. This Side Phase was prepared for development evaluation of parameters and mix times in preparation for the Active Phase used in future clinical batches.

### Final Compounding Phase

The Side Phase was added to the Emulsification Phase above with mixing at 375 RPM. Mixing continued at 480 RPM for 10 minutes while maintaining at 30° C. or below, final temperature was 28° C. A 2-oz sample was obtained for an in-process pH check. The pH was 7.1 (Range: 6.5-7.5). The phase was observed to be a white to off-white thin liquid emulsion.

### Reconciliation and Transfer to Holding

The batch net weight reconciled at 25.9 kg or 100% (range: 95-103%) with a 0.1 kg manufacturing loss. Once reconciled, the batch was transferred using a rotating lobe transfer pump (30 GPM) with a 200-mesh disk screen filter into a stainless-steel container (Capacity: 10-gal minimum) with clamp down lid for holding. Two (2) sets of 2-ounce samples in glass jars from each the Top Center, Middle Center and Bottom of the Holding Vessel were submitted to R&D for distribution to the appropriate testing sites as per Protocol 1110.001.020. Final weight transfer yield was 24.8 kg with a 1.1 kg transfer/sampling loss. For Lot SEZ-F, the 200-mesh disk filter was placed on the pump outlet side which may have contributed to the transfer loss.

### Process Description: 26.0 kg Pharmaceutical Rectal Foam Placebo, Lot SFT-F

### Primary Compounding Phase

The Primary Compounding Phase was prepared in a round bottom, jacketed, stainless steel Kettle (TK4896) (Capacity: 70 L) into which Purified Water USP/EP (19.2 kg) and Propylene Glycol USP (2.60 kg) were added. This was placed under a Cowles Dissolver (DS4879) with a 6-inch standard disperser blade. Mixing began at 374 RPM while Sodium Phosphate Dibasic USP (42.6 g), Sodium Dihydrogen Phosphate Monohydrate USP (30.3 g) and Disodium EDTA USP (13.0 g) were added. Mixing continued at 374 RPM for 10 minutes. Mixing was discontinued and the phase was observed to be a clear, colorless solution with no undissolved particles present. Using a stainless-steel container, 7.3 kg was transferred into a round bottom, jacketed stainless steel Kettle (TK4841) (Capacity: 10 L) for the side phase below.

A Mixing resumed with the remaining primary Compounding phase at 298 RPM while heating with steam in the kettle jacket to 76° C. (Range: 75-80° C.). Methylparaben NF (26.0 g) was added and mixing continued at 298 RPM while for 122 minutes and a final temperature 77° C. (Range: 75-80° C.). Mixing was discontinued and the phase was observed to be a clear, colorless solution with no undissolved particles present.

### Oil Phase

The Oil Phase was prepared in a round bottom, jacketed, stainless steel Kettle (TK4902) (Capacity: 10 L) into which Mineral Oil NF (1560 g), Isopropyl Myristate NF (130 g), White Petrolatum USP (260 g), Polyoxyl 20 Cetostearyl Ether NF (650 g), Cetyl Alcohol NF (260 g), Stearyl Alcohol NF (260 g) and Propylparaben NF (5.20 g) were added. This was placed under a 1.5 HP Cowles Dissolver (DS4855) with a 3-inch standard disperser blade. Mixing began at 414 RPM while heating with steam in the kettle jacket to 76° C. (Range: 75-80° C.) which took 25 minutes. Mixing continued at 414 RPM for 17 minutes and a final temperature 78° C. (Range: 75-80° C.). Mixing was discontinued and the phase was observed to be a clear, colorless solution with no undissolved particles present.

### Emulsification Phase

The Oil Phase was added to the Primary Compounding Phase above with mixing at 298 RPM. This was followed with a hot Purified Water USP/EP rinse of the Oil Phase Kettle and mixing increased to 375 RPM. Mixing continued at 375 RPM for 10 minutes while maintaining at 75-80° C., final temperature was 77° C. (Range: 75-80° C.). Mixing continued with ambient cooling water in the jacket at 375 RPM while cooling to 40° C. (Range: 40° C. or below) which took 10 minutes. Mixing continued at 375 RPM while cooling to 30° C. (Range: 30° C. or below) with chilled cooling water in the jacket which took 20 minutes. The phase was observed to be a white to off-white thin liquid emulsion.

### Side Phase

The Side Phase was prepared in a round bottom, jacketed, stainless steel Kettle (TK4841) (Capacity: 10 L) into which 7.3 kg of the Primary Compounding Phase was added. This was placed under a 1.5 HP Cowles Dissolver (DS4855) with a 3-inch standard disperser blade. Mixing began at 501 RPM while cooling with chilled cooling water in the kettle jacket to 21° C. (Range: 20-25° C.) which took 18 minutes. Mixing was discontinued and the phase was observed to be a clear, colorless solution with no undissolved particles present. This Side Phase was prepared for development evaluation of parameters and mix times in preparation for the Active Phase used in future clinical batches.

### Final Compounding Phase

The Side Phase was added to the Emulsification Phase above with mixing at 375 RPM. Mixing continued at 375 RPM for 10 minutes while maintaining at 30° C. or below, final temperature was 25° C. A 2-oz sample was obtained for an in-process pH check. The pH was 7.1 (Range: 6.5-7.5). The phase was observed to be a white to off-white thin liquid emulsion.

### Reconciliation and Transfer to Holding

The batch net weight reconciled at 25.0 kg or 96% (range: 95-103%) with a 1.0 kg manufacturing loss. Once reconciled, the batch was transferred using a rotating lobe transfer pump (30 GPM) with a 200-mesh disk screen filter into a stainless-steel container (Capacity: 10-gal minimum) with clamp down lid for holding. Due to the transfer loss observed for Lot SEZ-F, in Lot SFT-F the 200-mesh disk filter was placed on the kettle bottom valve. This allowed for the collection of bulk product from the transfer hoses post filter. Two (2) sets of 2-ounce samples in glass jars from each the Top Center, Middle Center and Bottom of the Holding Vessel were submitted to R&D for distribution to the appropriate testing sites as per Protocol 1110.001.020. Final weight transfer yield was 24.8 kg with a 0.2 kg transfer/sampling loss.

### Results

Lots SEZ-F and SFT-F preliminary bulk samples were taken after transfer to the holding container. Table 82 below provides the preliminary bulk physical test results for the Top and Bottom samples.

TABLE 82
Preliminary bulk physical results.

| | | Lot SEZ-F | | Lot SFT-F | |
|---|---|---|---|---|---|
| Test | Specification | Top | Bottom | Top | Bottom |
| Description | White to Off-White | Pass | Pass | Pass | Pass |
| | Thin Liquid | | | | |
| | Emulsion | | | | |
| pH | 6.5-7.5 (neat) | 7.0 | 7.0 | 7.1 | 7.1 |
| Specific | Report Results | 0.77 | 0.79 | 1.00 | 1.00 |
| Gravity | | | | | |

For Lots SEZ-F and SFT-F, preliminary bulk samples from the Top, Middle and Bottom were taken after transfer to the holding container for analytical preservative assay. Tables 83 and 84 below provides the preliminary bulk preservative assay results for the samples

TABLE 83
Bulk analytical results for Lot SEZ-F Lot SEZ-F

| | Sample | Methylparaben | Propylparaben |
|---|---|---|---|
| Location | # | % Formulation | % Formulation |
| Top Center | 1 | 101.8 | 107.9 |
| | 2 | 103.2 | 109.3 |
| | 3 | 101.6 | 107.7 |
| | Mean | 102.2 | 108.3 |
| | % RSD | 0.82 | 0.80 |
| Middle | 1 | 102.9 | 109.1 |
| Center | 2 | 101.9 | 108.0 |
| | 3 | 102.7 | 109.1 |
| | Mean | 102.5 | 108.7 |
| | % RSD | 0.57 | 0.59 |
| Bottom | 1 | 98.8 | 104.7 |
| | 2 | 101.3 | 107.4 |
| | 3 | 100.2 | 106.3 |
| | Mean | 100.1 | 106.1 |
| | % RSD | 1.26 | 1.29 |

TABLE 84
Bulk analytical results for Lot SEZ-F
Lot SFT-F

| | Sample | Methylparaben | Propylparaben |
|---|---|---|---|
| Location | # | % Formulation | % Formulation |
| Top Center | 1 | 102.2 | 103.7 |
| | 2 | 103.2 | 104.7 |
| | 3 | 102.4 | 103.9 |
| | Mean | 102.6 | 104.1 |
| | % RSD | 0.51 | 0.50 |
| Middle | 1 | 101.8 | 103.2 |
| Center | 2 | 103.0 | 104.5 |
| | 3 | 103.0 | 104.5 |
| | Mean | 102.6 | 104.1 |
| | % RSD | 0.7 | 0.7 |
| Bottom | 1 | 103.0 | 104.4 |
| | 2 | 103.1 | 104.6 |
| | 3 | 103.1 | 104.6 |
| | Mean | 103.1 | 104.5 |
| | % RSD | 0.09 | 0.12 |

For Lot SEZ-F, the preliminary bulk assay results for Propylparaben were observed to be high but still within specification 90.0 to 110.0% Formula for the bulk product. This may be due to analytical method variability, but no formal investigation was performed to determine root cause since these results were still in specification.

Lots SEZ-F and SFT-F were packaged on the Aerosol line into 30 g cans. Each can contained a targeted bulk product weight of 31.0 g and a targeted 3.4 g of Aeropin 35 propellant. Samples were collected from the beginning, middle and end of the packaging run. Table 85 below provides the preservative assay results for the end of run cans for Lots SEZ-F and SFT-F. Testing was only performed on the end can with the beginning and middle cans held for retains.

TABLE 85
End of run can analytical results. Medicated
Foam Placebo Finished Product

| | | | |
|---|---|---|---|
| | | Methylparaben | Propylparaben |
| Location | Sample # | % Formulation | % Formulation |

Lot SEZ-F

| | | | |
|---|---|---|---|
| End | 1 | 101.6 | 107.3 |
| | 2 | 101.6 | 107.2 |
| | 3 | 101.8 | 107.5 |
| | Mean | 101.7 | 107.4 |
| | % RSD | 0.14 | 0.16 |

Lot SFT-F

| | | | |
|---|---|---|---|
| End | 1 | 103.4 | 104.9 |
| | 2 | 103.2 | 104.6 |
| | 3 | 101.5 | 103.0 |
| | Mean | 102.7 | 104.2 |
| | % RSD | 1.00 | 0.96 |

For Lot SEZ-F, the end of run packaged assay results for Propylparaben were observed to be high but still within specification 90.0 to 110.0% Formula for the product. As mentioned above, this may be due to analytical variability, but no formal investigation was performed to determine root cause since these results were still in specification. Three cans from the Beginning, four cans from the Middle and three cans from the End of the packaging run were provided to R&D for Physical Actuation testing. Each can was actuated for a total of 28 actuations and the weights recorded until the can was exhausted. For the study, 21 actuations including the initial actuation were used for total mass dispensed and used to calculate the average actuation amount. The actuation for each can was performed as follows: the can was equilibrated in a 25° C. water batch for at least 30 minutes. The can was removed and initially shaken for 30 seconds prior to the first actuation. There was no purge of the initial actuation. For each actuation the can was inverted, the meter head pressed for at least 5 seconds and then slowly released. This was followed with 4 more actuations. The can was placed back into the 25° C. water bath for at least 5 minutes before the next set of 5 actuations were performed. One actuation sample from each can was collected to perform the pH on the foam product. Table 86 below provides a summary of the finished physical test results for Lot SEZ-F. The actuation data for both Lots is provided below.

TABLE 86
Finished Physical Test Results for Lot SEZ-F.

| | | Beg 1 | Beg 2 | Beg 3 | Mid 1 | Mid 2 | Mid 3 | Mid 4 | End 1 | End 2 | End 3 | |
|---|---|---|---|---|---|---|---|---|---|---|---|---|
| Description | White to off-white | Pass | Pass | Pass | Pass | Pass | Pass | Pass | Pass | Pass | Pass | |
| | foam | | | | | | | | | | | |
| pH | 6.5 to 7.5 (1 | 6.9 | 7.0 | 7.0 | 6.9 | 6.9 | 6.8 | 6.8 | 6.9 | 6.7 | 6.9 | Total |
| | actuation dissolved | | | | | | | | | | | Average |
| | in 100 mL 5% | | | | | | | | | | | |
| | Methanol/95% | | | | | | | | | | | |
| | Water solution) | | | | | | | | | | | |
| Product | total mass for 21 | 22.69 | 23.73 | 22.96 | 23.77 | 23.23 | 23.25 | 24.08 | 28.33 | 22.20 | 23.23 | 23.75 |
| Dispensed | actuations (g) | | | | | | | | | | | |
| Average | Amount (g) | 1.08 | 1.07 | 1.09 | 1.13 | 1.11 | 1.11 | 1.14 | 1.05 | 1.06 | 1.11 | 1.10 |
| Actuation | (N = 21) | | | | | | | | | | | |
| | % RSD | 7.9 | 7.0 | 7.1 | 5.8 | 5.5 | 4.5 | 5.8 | 11.4 | 6.8 | 6.3 | 7.0 |

Beg = beginning. Mid = middle.

TABLE 87
Finished Physical Test Results for Lot SFT-F.

| | | Beg 1 | Beg 2 | Beg 3 | Mid 1 | Mid 2 | Mid 3 | Mid 4 | End 1 | End 2 | End 3 | |
|---|---|---|---|---|---|---|---|---|---|---|---|---|
| Description | White to off-white | Pass | Pass | Pass | Pass | Pass | Pass | Pass | Pass | Pass | Pass | |
| | foam | | | | | | | | | | | |
| pH | 6.5 to 7.5 (1 | 6.8 | 6.9 | 7.1 | 6.9 | 7.1 | 6.8 | 7.1 | 7.1 | 6.8 | 6.8 | Total |
| | actuation dissolved | | | | | | | | | | | Average |
| | in 100 mL 5% | | | | | | | | | | | |
| | Methanol/95% | | | | | | | | | | | |
| | Water solution) | | | | | | | | | | | |
| Product | total mass for 21 | 26.00 | 26.75 | 27.35 | 26.80 | 26.84 | 25.87 | 26.75 | 25.81 | 26.94 | 25.47 | 26.43 |
| Dispensed | actuations (g) | | | | | | | | | | | |
| Average | Amount (g) | 1.24 | 1.27 | 1.30 | 1.28 | 1.28 | 1.23 | 1.24 | 1.19 | 1.28 | 1.21 | 1.26 |
| Actuation | (N = 21) | | | | | | | | | | | |
| | % RSD | 6.1 | 21.0 | 4.0 | 3.6 | 3.7 | 6.2 | 6.3 | 6.8 | 4.0 | 7.3 | 8.6 |

Beg = beginning. Mid = middle.

For Lot SEZ-F, Beginning Can #2 had the 11th dispensed amount at 2.25 g. This far exceeded the 1.35 mL dome volume and was not calculated in the Average Actuation and % RSD recorded in the table above. No investigation was performed since this was discovered after testing had been completed and may have been a weighing error.

For Lot SFT-F, three cans from the Beginning, four cans from the Middle and three cans from the End of the packaging run were provided to R&D for Physical Actuation testing. The cans for Lot SFT-F were actuated as described above. Table 88 below provides a summary of the finished physical test results for Lot SFT-F. The Beginning Can #2 for Lot SFT-F provided inconsistent actuation weights when compared to the others from the Lot. While no formal investigation was performed, it may have been due to a defective meter head.

Additional actuations beyond the initial 21 actuations were performed until the can was exhausted to determine if dosing beyond 21 actuations were possible. Tables 88 and 89 below provide additional actuations weight results for Lots SEZ-F and SFT-F, respectively.

TABLE 88
Additional actuation weights until can exhausted for Lot SEZ-F

| | Beginning -1 | | Beginning - 2 | | Beginning - 3 | | Middle-1 | | Middle- 2 | |
|---|---|---|---|---|---|---|---|---|---|---|
| | | Can | | Can | | Can | | Can | | Can |
| | Can | weight | Can | weight | Can | weight | Can | weight | Can | weight |
| Actuation | weight | difference | weight | difference | weight | difference | weight | difference | weight | difference |
| # | (g) | (g) | (g) | (g) | (g) | (g) | (g) | (g) | (g) | (g) |
| 0 | 32.89 | | 32.31 | | 32.71 | | 31.81 | | 32.82 | |
| 22 | 31.88 | 1.01 | 31.21 | 1.10 | 31.69 | 1.02 | 30.68 | 1.13 | 31.70 | 1.12 |
| 23 | 30.67 | 1.21 | 30.04 | 1.17 | 30.55 | 1.14 | 29.40 | 1.28 | 30.49 | 1.21 |
| 24 | 29.54 | 1.13 | 28.91 | 1.13 | 29.39 | 1.16 | 28.25 | 1.15 | 29.37 | 1.12 |
| 25 | 28.45 | 1.09 | 27.88 | 1.03 | 28.35 | 1.04 | 27.17 | 1.08 | 28.36 | 1.01 |
| 26 | 27.46 | 60.99 | 26.85 | 1.03 | 27.41 | 0.94 | 26.13 | 1.04 | 27.44 | 0.92 |
| 27 | 26.48 | 0.98 | 25.90 | 0.95 | 26.39 | 1.02 | 25.37 | 0.76 | 26.54 | 0.90 |
| 28 | 25.54 | 0.94 | | | 25.46 | 0.93 | 24.76 | 0.61 | 25.85 | 0.69 |
| | Middle- 3 | | Middle- 4 | | End - 1 | | End - 2 | | End - 3 | |
| | | Can | | Can | | Can | | Can | | Can |
| | Can | weight | Can | weight | Can | weight | Can | weight | Can | weight |
| Actuation | weight | difference | weight | difference | weight | difference | weight | difference | weight | difference |
| # | (g) | (g) | (g) | (g) | (g) | (g) | (g) | (g) | (g) | (g) |
| 0 | 31.27 | | 31.82 | | 32.67 | | 31.14 | | 32.00 | |
| 22 | 30.19 | 1.08 | 30.71 | 1.11 | 31.63 | 1.04 | 30.17 | 0.97 | 30.91 | 1.09 |
| 23 | 29.02 | 1.17 | 29.53 | 1.18 | 30.47 | 1.16 | 28.98 | 1.19 | 29.71 | 1.20 |
| 24 | 27.90 | 1.12 | 28.37 | 1.16 | 29.39 | 1.08 | 27.92 | 1.06 | 28.68 | 1.03 |
| 25 | 26.94 | 0.96 | 27.26 | 1.11 | 28.41 | 0.98 | 27.04 | 0.88 | 27.76 | 0.92 |
| 26 | 26.07 | 0.87 | 26.55 | 0.71 | 27.37 | 1.04 | 26.44 | 0.60 | 26.81 | 0.95 |
| 27 | 25.26 | 0.81 | 25.80 | 0.75 | 26.44 | 0.93 | | | 25.90 | 0.91 |
| 28 | 24.71 | 0.55 | 25.20 | 0.60 | 25.76 | 0.68 | | | | |

TABLE 89
Additional actuation weights until can exhausted for Lot SFT-F
IW-3300 Medicated Foam Placebo Lot SFT-F

| | Beginning -1 | | Beginning - 2 | | Beginning - 3 | | Middle-1 | | Middle- 2 | |
|---|---|---|---|---|---|---|---|---|---|---|
| | | Can | | Can | | Can | | Can | | Can |
| | Can | weight | Can | weight | Can | weight | Can | weight | Can | weight |
| Actuation | weight | difference | weight | difference | weight | difference | weight | difference | weight | difference |
| # | (g) | (g) | (g) | (g) | (g) | (g) | (g) | (g) | (g) | (g) |
| 0 | 27.41 | | 27.55 | | 26.43 | | 29.58 | | 29.01 | |
| 22 | 26.33 | 1.08 | 26.51 | 1.04 | 25.29 | 1.14 | 28.54 | 1.04 | 28.03 | 0.98 |
| 23 | 25.24 | 1.09 | 25.38 | 1.13 | 24.42 | 0.87 | 27.35 | 1.19 | 26.97 | 1.06 |
| 24 | 24.38 | 0.86 | 24.55 | 0.83 | 23.79 | 0.63 | 26.40 | 0.95 | 26.15 | 0.82 |
| 25 | 23.68 | 0.70 | 23.73 | 0.82 | | | 25.85 | 0.55 | 25.56 | 0.59 |
| 26 | 23.19 | 0.49 | 23.23 | 0.50 | | | | | | |
| | Middle- 3 | | Middle- 4 | | End -1 | | End - 2 | | End - 3 | |
| | | Can | | Can | | Can | | Can | | Can |
| | Can | weight | Can | weight | Can | weight | Can | weight | Can | weight |
| Actuation | weight | difference | weight | difference | weight | difference | weight | difference | weight | difference |
| # | (g) | (g) | (g) | (g) | (g) | (g) | (g) | (g) | (g) | (g) |
| 0 | 28.71 | | 28.93 | | 29.92 | | 29.13 | | 30.43 | |
| 22 | 27.57 | 1.14 | 27.75 | 1.18 | 28.73 | 1.19 | 27.94 | 1.19 | 29.32 | 1.11 |
| 23 | 26.45 | 1.12 | 26.62 | 1.13 | 27.58 | 1.15 | 26.81 | 1.13 | 28.22 | 1.10 |
| 24 | 25.50 | 0.95 | 25.59 | 1.03 | 26.52 | 1.06 | 26.01 | 0.80 | 27.27 | 0.95 |
| 25 | 24.64 | 0.86 | 24.77 | 0.82 | 25.57 | 0.95 | | | 26.26 | 1.01 |
| 26 | | | | | | 24.8 | | | 25.38 | 0.88 |
| | | | | | | 60.71 | | | | |

### CONCLUSION

The manufacturing of pharmaceutical rectal foam composition Lots SEZ-F and SFT-F at the 26.0 kg production scale was deemed successful. Lot SEZ-F had more air entrapped (aerated) than Lot SFT-F. This was apparent based on the low specific gravity observed for Lot SEZ-F, 0.77 and 0.79 (see above). For Lot SFT-F the specific gravity was 1.00. The aeration in Lot SEZ-F influenced the filling on the Aerosol line and led to the lower amount of foam actuated as compared to Lot SFT-F. While the mixing parameters for Lots SEZ-F and SFT-F were similar, the major difference was observed in Step C.V.b. This is the step after addition of side aqueous phase to the main compounding vessel. In Lot SEZ-F the mixing speed was 480 RPM while for Lot SFT-F the mixing speed was lower at 375 RPM. Based on the actuation results obtained from Lots SEZ-F and SFT-F, the delivered dose actuation amount is 1.18 g=10%. Based on the actuation data obtained above, the 21 actuations (1 to 21) are number of usable doses obtained.

TABLE 90
Proposed clinical formulation.

| | Proposed Formula for | | | | Proposed Formula for | | | | Proposed Formula for | | | |
|---|---|---|---|---|---|---|---|---|---|---|---|---|
| | Placebo | | | | 100 μg/dose | | | | 300 μg/dose | | | |
| | Theoretical | | | | Theoretical | | | | Theoretical | | | |
| | | Amounts | | | | Amounts | | | | Amounts | | |
| | | per | | per | | per | | per | | per | | per |
| Prototype A | Bulk | 26.0 kg | | Actuation | Bulk % | 26.0 kg | | Actuation | Bulk % | 26.0 kg | | Actuation |
| Ingredients | % w/w | batch | | (μg) | w/w | batch | | (μg) | w/w | batch | | (μg) |
| Purified Water | 77.5495 | 20.2 | kg | 803675 | 77.5399 | 20.2 | kg | 803575 | 77.5207 | 20.2 | kg | 803376 |
| USP/EP | | | | | | | | | | | | |
| Propylene | 10.0000 | 2.60 | kg | 103634 | 10.0000 | 2.60 | kg | 103634 | 10.0000 | 2.60 | kg | 103634 |
| Glycol USP | | | | | | | | | | | | |
| Sodium | 0.1640 | 42.6 | g | 1700 | 0.1640 | 42.6 | g | 1700 | 0.1640 | 42.6 | g | 1700 |
| Phosphate | | | | | | | | | | | | |
| Dibasic USP | | | | | | | | | | | | |
| Sodium | 0.1165 | 30.3 | g | 1207 | 0.1165 | 30.3 | g | 1207 | 0.1165 | 30.3 | g | 1207 |
| Dihydrogen | | | | | | | | | | | | |
| Phosphate | | | | | | | | | | | | |
| Monohydrate | | | | | | | | | | | | |
| USP | | | | | | | | | | | | |
| Disodium | 0.0500 | 13.0 | g | 518 | 0.0500 | 13.0 | g | 518 | 0.0500 | 13.0 | g | 518 |
| EDTA USP | | | | | | | | | | | | |
| Methylparaben | 0.1000 | 26.0 | g | 1036 | 0.1000 | 26.0 | g | 1036 | 0.1000 | 26.0 | g | 1036 |
| NF | | | | | | | | | | | | |
| Peptide | | | | | 0.00961 | 2.50 | g | 100 | 0.02883 | 7.50 | g | 300 |
| Light Mineral | 6.0000 | 1560 | g | 62180 | 6.0000 | 1560 | g | 62180 | 6.0000 | 1560 | g | 62180 |
| Oil NF | | | | | | | | | | | | |
| Isopropyl | 0.5000 | 130 | g | 5182 | 0.5000 | 130 | g | 5182 | 0.5000 | 130 | g | 5182 |
| Myristate NF | | | | | | | | | | | | |
| White | 1.0000 | 260 | g | 10363 | 1.0000 | 260 | g | 10363 | 1.0000 | 260 | g | 10363 |
| Petrolatum | | | | | | | | | | | | |
| USP | | | | | | | | | | | | |
| Polyoxyl 20 | 2.5000 | 650 | g | 25908 | 2.5000 | 650 | g | 25908 | 2.5000 | 650 | g | 25908 |
| Cetostearyl | | | | | | | | | | | | |
| Ether NF | | | | | | | | | | | | |
| Cetyl Alcohol | 1.0000 | 260 | g | 10363 | 1.0000 | 260 | g | 10363 | 1.0000 | 260 | g | 10363 |
| NF | | | | | | | | | | | | |
| Stearyl | 1.0000 | 260 | g | 10363 | 1.0000 | 260 | g | 10363 | 1.0000 | 260 | g | 10363 |
| Alcohol NF | | | | | | | | | | | | |
| Propylparaben | 0.0200 | 5.20 | g | 207 | 0.0200 | 5.20 | g | 207 | 0.0200 | 5.20 | g | 207 |
| NF | | | | | | | | | | | | |
| Total | 100.00 | 26.0 | kg | | 100.00 | 26.0 | kg | | 100.00 | 26.0 | kg | |
| AP35 | | 3.4 | g | 113663 | | 3.4 | g | 113663 | | 3.4 | g | 113663 |
| Propellant | | | | | | | | | | | | |
| Total | | | | 1150000 | | | | 1150000 | | | | 1150001 |

TABLE 91
Packaging components.
Packaging Component

Can, 30 g 35 mm × 65 mm
Valve, CV/1″ Nitrile AR/PP
Actuator, Metered Head 1.35 mL
Foam Shield, Lablabo
Aeropin 35, 120 Gallon
SHP, ×40 Aerosol Blank
DIC (×40) Aerosol (×1/SHP)
PAD, Spring Corrugate (×2/SHP)
SHPR LBL, 3 × 5 BLANK

The Tables below show the actuation rate raw data for Lots SEZ-F and SFT-F.

TABLE 92
Lot SEZ-F actuation weight raw data.

| | Beginning - 1 | | | Beginning - 2 | | | Beginning - 3 | | |
|---|---|---|---|---|---|---|---|---|---|
| | pH = 6.9 | | | pH = 7.0 | | | pH = 7.0 | | |
| | | Can | | | Can | | | Can | |
| | Can | weight | Foam | Can | weight | Foam | Can | weight | Foam |
| Actuation | weight | difference | Height | weight | difference | Height | weight | difference | Height |
| # | (g) | (g) | (mL) | (g) | (g) | (mL) | (g) | (g) | (mL) |
| 0 | 56.07 | | | 56.17 | | | 55.89 | | |
| 1 | 55.08 | 0.99 | 19 | 54.96 | 1.21 | 24 | 54.65 | 1.24 | 26 |
| 2 | 53.96 | 1.12 | | 53.80 | 1.16 | | 53.56 | 1.09 | |
| 3 | 52.83 | 1.13 | | 52.67 | 1.13 | | 52.48 | 1.08 | |
| 4 | 51.78 | 1.05 | | 51.56 | 1.11 | | 51.39 | 1.09 | |
| 5 | 50.70 | 1.08 | | 50.49 | 1.07 | | 50.40 | 0.99 | |
| 6 | 49.70 | 1.00 | | 49.47 | 1.02 | | 49.36 | 1.04 | |
| 7 | 48.73 | 0.97 | | 48.47 | 1.00 | | 48.38 | 0.98 | |
| 8 | 47.81 | 0.92 | | 47.52 | 0.95 | | 47.40 | 0.98 | |
| 9 | 46.90 | 0.91 | | 46.60 | 0.92 | | 46.41 | 0.99 | |
| 10 | 45.86 | 1.04 | 26 | 45.51 | 1.09 | 37 | 45.4 | 1.01 | 34 |
| 11 | 44.66 | 1.20 | | 43.26 | 2.25 | | 44.2 | 1.20 | |
| 12 | 43.48 | 1.18 | | 42.14 | 1.12 | | 43.05 | 1.15 | |
| 13 | 42.32 | 1.16 | | 41.06 | 1.08 | | 41.93 | 1.12 | |
| 14 | 41.20 | 1.12 | | 40.01 | 1.05 | | 40.9 | 1.03 | |
| 15 | 40.12 | 1.08 | | 38.92 | 1.09 | | 39.74 | 1.16 | |
| 16 | 38.99 | 1.13 | | 37.91 | 1.01 | | 38.63 | 1.11 | |
| 17 | 37.93 | 1.06 | | 36.89 | 1.02 | | 37.54 | 1.09 | |
| 18 | 36.83 | 1.10 | | 35.84 | 1.05 | | 36.45 | 1.09 | |
| 19 | 35.73 | 1.10 | | 34.74 | 1.10 | | 35.27 | 1.18 | |
| 20 | 34.52 | 1.21 | 37 | 33.54 | 1.20 | 38 | 34.09 | 1.18 | 37 |
| 21 | 33.38 | 1.14 | | 32.44 | 1.10 | | 32.93 | 1.16 | |
| 0 | 32.89 | | | 32.31 | | | 32.71 | | |
| 22 | 31.88 | 1.01 | | 31.21 | 1.10 | | 31.69 | 1.02 | |
| 23 | 30.67 | 1.21 | | 30.04 | 1.17 | | 30.55 | 1.14 | |
| 24 | 29.54 | 1.13 | | 28.91 | 1.13 | | 29.39 | 1.16 | |
| 25 | 28.45 | 1.09 | | 27.88 | 1.03 | | 28.35 | 1.04 | |
| 26 | 27.46 | 0.99 | | 26.85 | 1.03 | | 27.41 | 0.94 | |
| 27 | 26.48 | 0.98 | | 25.90 | 0.95 | | 26.39 | 1.02 | |
| 28 | 25.54 | 0.94 | | | | | 25.46 | 0.93 | |
| | Total | 22.69 | | Total | 23.73 | | Total | 22.96 | |
| | Mean | 1.08 | | Mean | 1.07 | | Mean | 1.09 | |
| | % RSD | 7.9 | | % RSD | 7.0 | | % RSD | 7.1 | |

| | Middle -1 | | | Middle - 2 | | |
|---|---|---|---|---|---|---|
| | pH = 6.9 | | | pH = 6.9 | | |
| | | Can | | | Can | |
| | Can | weight | Foam | Can | weight | Foam |
| Actuation | weight | difference | Height | weight | difference | Height |
| # | (g) | (g) | (mL) | (g) | (g) | (mL) |
| 0 | 55.74 | | | 56.33 | | |
| 1 | 54.49 | 1.25 | 26 | 55.22 | 1.11 | 27 |
| 2 | 53.27 | 1.22 | | 54.04 | 1.18 | |
| 3 | 52.09 | 1.18 | | 52.87 | 1.17 | |
| 4 | 50.94 | 1.15 | | 51.74 | 1.13 | |
| 5 | 49.83 | 1.11 | | 50.66 | 1.08 | |
| 6 | 48.75 | 1.08 | | 49.61 | 1.05 | |
| 7 | 47.70 | 1.05 | | 48.63 | 0.98 | |
| 8 | 46.68 | 1.02 | | 47.63 | 1.00 | |
| 9 | 45.64 | 1.04 | | 46.65 | 0.98 | |
| 10 | 44.41 | 1.23 | 35 | 45.50 | 1.15 | 35 |
| 11 | 43.28 | 1.13 | | 44.35 | 1.15 | |
| 12 | 42.11 | 1.17 | | 43.21 | 1.14 | |
| 13 | 41.00 | 1.11 | | 42.11 | 1.10 | |
| 14 | 39.86 | 1.14 | | 41.00 | 1.11 | |
| 15 | 38.71 | 1.15 | | 39.89 | 1.11 | |
| 16 | 37.60 | 1.11 | | 38.77 | 1.12 | |
| 17 | 36.54 | 1.06 | | 37.68 | 1.09 | |
| 18 | 35.45 | 1.09 | | 36.56 | 1.12 | |
| 19 | 34.27 | 1.18 | | 35.42 | 1.14 | |
| 20 | 33.06 | 1.21 | 38 | 34.21 | 1.21 | 40 |
| 21 | 31.97 | 1.09 | | 33.10 | 1.11 | |
| 0 | 31.81 | | | 32.82 | | |
| 22 | 30.68 | 1.13 | | 31.70 | 1.12 | |
| 23 | 29.40 | 1.28 | | 30.49 | 1.21 | |
| 24 | 28.25 | 1.15 | | 29.37 | 1.12 | |
| 25 | 27.17 | 1.08 | | 28.36 | 1.01 | |
| 26 | 26.13 | 1.04 | | 27.44 | 0.92 | |
| 27 | 25.37 | 0.76 | | 26.54 | 0.90 | |
| 28 | 24.76 | 0.61 | | 25.85 | 0.69 | |
| | Total | 23.77 | | Total | 23.23 | |
| | Mean | 1.13 | | Mean | 1.11 | |
| | % RSD | 5.8 | | % RSD | 5.5 | |

TABLE 93
Lot SEZ-F actuation weight raw data.

| | Middle - 3 | | | Middle - 4 | | | End -1 | | |
|---|---|---|---|---|---|---|---|---|---|
| | pH = 6.8 | | | pH = 6.8 | | | pH = 6.9 | | |
| | | Can | | | Can | | | Can | |
| | Can | weight | Foam | Can | weight | Foam | Can | weight | Foam |
| Actuation | weight | difference | Height | weight | difference | Height | weight | difference | Height |
| # | (g) | (g) | (mL) | (g) | (g) | (mL) | (g) | (g) | (mL) |
| 0 | 54.55 | | | 55.95 | | | 55.26 | | |
| 1 | 53.45 | 1.10 | 22 | 54.67 | 1.28 | 25 | 54.07 | 1.19 | 26 |
| 2 | 52.28 | 1.17 | | 53.39 | 1.28 | | 52.95 | 1.12 | |
| 3 | 51.12 | 2.26 | | 52.19 | 1.20 | | 51.86 | 1.09 | |
| 4 | 50.00 | 1.12 | | 51.03 | 1.16 | | 50.85 | 1.01 | |
| 5 | 48.92 | 1.08 | | 49.87 | 1.16 | | 49.87 | 0.98 | |
| 6 | 47.85 | 1.07 | | 48.75 | 1.12 | | 48.93 | 0.94 | |
| 7 | 46.82 | 1.03 | | 47.65 | 1.10 | | 48.02 | 0.91 | |
| 8 | 45.80 | 1.02 | | 46.58 | 1.07 | | 47.14 | 0.88 | |
| 9 | 44.79 | 1.01 | | 45.53 | 1.05 | | 46.24 | 0.90 | |
| 10 | 43.71 | 1.08 | 36 | 44.33 | 1.20 | 42 | 45.20 | 1.04 | 38 |
| 11 | 42.54 | 1.17 | | 43.08 | 1.25 | | 44.00 | 1.20 | |
| 12 | 41.42 | 1.12 | | 41.92 | 1.16 | | 42.91 | 1.09 | |
| 13 | 40.28 | 1.14 | | 40.82 | 1.10 | | 41.76 | 1.15 | |
| 14 | 39.11 | 1.17 | | 39.72 | 1.10 | | 40.56 | 1.20 | |
| 15 | 38.02 | 1.09 | | 38.60 | 1.12 | | 39.44 | 1.12 | |
| 16 | 36.96 | 1.06 | | 37.51 | 1.09 | | 38.29 | 1.15 | |
| 17 | 35.84 | 1.12 | | 36.45 | 1.06 | | 37.19 | 1.10 | |
| 18 | 34.69 | 1.15 | | 35.41 | 1.04 | | 36.06 | 1.13 | |
| 19 | 33.59 | 1.10 | | 34.26 | 1.15 | | 34.91 | 1.15 | |
| 20 | 32.45 | 1.14 | 38 | 33.08 | 1.18 | 38 | 33.82 | 1.09 | 35 |
| 21 | 31.30 | 1.15 | | 31.87 | 1.21 | | 32.69 | 1.13 | |
| 0 | 31.27 | | | 31.82 | | | 32.67 | | |
| 22 | 30.19 | 1.08 | | 30.71 | 1.11 | | 31.63 | 1.04 | |
| 23 | 29.02 | 1.17 | | 29.53 | 1.18 | | 30.47 | 1.16 | |
| 24 | 27.90 | 1.12 | | 28.37 | 1.16 | | 29.39 | 1.08 | |
| 25 | 26.94 | 0.96 | | 27.26 | 1.11 | | 28.41 | 0.98 | |
| 26 | 26.07 | 0.87 | | 26.55 | 0.71 | | 27.37 | 1.04 | |
| 27 | 25.26 | 0.81 | | 25.80 | 0.75 | | 26.44 | 0.93 | |
| 28 | 24.71 | 0.55 | | 25.20 | 0.60 | | 25.76 | 0.68 | |
| | Total | 23.25 | | Total | 24.08 | | Total | 28.33 | |
| | Mean | 1.11 | | Mean | 1.14 | | Mean | 1.05 | |
| | % RSD | 4.5 | | % RSD | 5.8 | | % RSD | 11.4 | |
| | Mean | 1.10 | | Average delivered amount (g) per actuation (1 | | | | | |
| | % RSD | 7.0 | | | | | | | |

| | | |
|---|---|---|
| Total | 23.75 | Average Total product (g) delivered for 21 actuation (1 thru 21) |

| | End - 2 | | | End - 3 | | |
|---|---|---|---|---|---|---|
| | pH = 6.7 | | | pH = 6.9 | | |
| | | Can | | | Can | |
| | Can | weight | Foam | Can | weight | Foam |
| Actuation | weight | difference | Height | weight | difference | Height |
| # | (g) | (g) | (mL) | (g) | (g) | (mL) |
| 0 | 53.34 | | | 55.29 | | |
| 1 | 52.38 | 0.96 | 26 | 54.02 | 1.27 | 26 |
| 2 | 51.28 | 1.10 | | 52.83 | 1.19 | |
| 3 | 50.08 | 1.20 | | 51.67 | 1.16 | |
| 4 | 48.96 | 1.12 | | 50.55 | 1.12 | |
| 5 | 47.95 | 1.01 | | 49.48 | 1.07 | |
| 6 | 46.86 | 1.09 | | 48.40 | 1.08 | |
| 7 | 45.86 | 1.00 | | 47.35 | 1.05 | |
| 8 | 44.90 | 0.96 | | 46.34 | 1.01 | |
| 9 | 43.97 | 0.93 | | 45.3 | 1.04 | |
| 10 | 42.88 | 1.09 | 28 | 44.25 | 1.05 | 32 |
| 11 | 41.73 | 1.15 | | 43.21 | 1.04 | |
| 12 | 40.71 | 1.02 | | 42.18 | 1.03 | |
| 13 | 39.65 | 1.06 | | 41.05 | 1.13 | |
| 14 | 38.58 | 1.07 | | 39.96 | 1.09 | |
| 15 | 37.52 | 1.06 | | 38.85 | 1.11 | |
| 16 | 36.49 | 1.03 | | 37.76 | 1.09 | |
| 17 | 35.46 | 1.03 | | 36.70 | 1.06 | |
| 18 | 34.49 | 0.97 | | 35.61 | 1.09 | |
| 19 | 33.41 | 1.08 | | 34.48 | 1.13 | |
| 20 | 32.30 | 1.11 | 34 | 33.24 | 1.24 | 40 |
| 21 | 31.14 | 2.26 | | 32.06 | 1.18 | |
| 0 | 31.14 | | | 32.00 | | |
| 22 | 30.17 | 0.97 | | 30.91 | 1.09 | |
| 23 | 28.98 | 1.19 | | 29.71 | 1.20 | |
| 24 | 27.92 | 1.06 | | 28.68 | 1.03 | |
| 25 | 27.04 | 0.88 | | 27.76 | 0.92 | |
| 26 | 26.44 | 0.60 | | 26.81 | 0.95 | |
| 27 | | | | 25.90 | 0.91 | |
| 28 | | | | | | |
| | Total | 22.20 | | Total | 23.23 | |
| | Mean | 1.06 | | Mean | 1.11 | |
| | % RSD | 6.8 | | % RSD | 6.3 | |

Average delivered amount (g) per actuation (1
Average Total product (g) delivered for 21 actuation (1 thru 21)

TABLE 94
Lot SFT-F actuation weight raw data.
IW-3300 Medicated Foam Placebo Lot SFT-F

| | Beginning -1 | | | Beginning - 2 | | | Beginning - 3 | | |
|---|---|---|---|---|---|---|---|---|---|
| | pH = 6.8 | | | pH = 6.9 | | | pH = 7.1 | | |
| | | Can | | | Can | | | Can | |
| | Can | weight | Foam | Can | weight | Foam | Can | weight | Foam |
| Actuation | weight | difference | Height | weight | difference | Height | weight | difference | Height |
| # | (g) | (g) | (mL) | (g) | (g) | (mL) | (g) | (g) | (mL) |
| 0 | 54.45 | | | 55.46 | | | 56.10 | | |
| 1 | 53.25 | 1.20 | 26 | 53.69 | 1.77 | 30 | 54.87 | 1.23 | 31 |
| 2 | 52.00 | 1.25 | | 52.40 | 1.29 | | 53.58 | 1.29 | |
| 3 | 50.71 | 1.29 | | 51.10 | 1.30 | | 52.28 | 1.30 | |
| 4 | 49.35 | 1.36 | | 49.80 | 1.30 | | 50.95 | 1.33 | |
| 5 | 48.09 | 1.26 | | 48.47 | 1.33 | | 49.61 | 1.34 | |
| 6 | 46.70 | 1.39 | | 47.14 | 1.33 | | 48.27 | 1.34 | |
| 7 | 45.49 | 1.21 | | 45.81 | 1.33 | | 46.91 | 1.36 | |
| 8 | 44.29 | 1.20 | | 44.48 | 1.33 | | 45.58 | 1.33 | |
| 9 | 43.04 | 1.25 | | 43.17 | 1.31 | | 44.21 | 1.37 | |
| 10 | 41.70 | 1.34 | 43 | 41.19 | 1.98 | 43 | 42.9 | 1.31 | 46 |
| 11 | 40.43 | 1.27 | | 40.66 | 0.53 | | 41.53 | 1.37 | |
| 12 | 39.19 | 1.24 | | 39.36 | 1.30 | | 40.35 | 1.18 | |
| 13 | 37.98 | 1.21 | | 38.09 | 1.27 | | 39.00 | 1.35 | |
| 14 | 36.69 | 1.29 | | 36.93 | 1.16 | | 37.65 | 1.35 | |
| 15 | 35.45 | 1.24 | | 35.65 | 1.28 | | 36.35 | 1.30 | |
| 16 | 34.28 | 1.17 | | 34.42 | 1.23 | | 35.04 | 1.31 | |
| 17 | 33.07 | 1.21 | | 33.23 | 1.19 | | 33.76 | 1.28 | |
| 18 | 31.97 | 1.10 | | 32.11 | 1.12 | | 32.50 | 1.26 | |
| 19 | 30.69 | 1.28 | | 30.93 | 1.18 | | 31.27 | 1.23 | |
| 20 | 29.55 | 1.14 | 36 | 29.77 | 1.16 | 38 | 30.00 | 1.27 | 42 |
| 21 | 28.45 | 1.10 | | 28.71 | 1.06 | | 28.75 | 1.25 | |
| 0 | 27.41 | | | 27.55 | | | 26.43 | | |
| 22 | 26.33 | 1.08 | | 26.51 | 1.04 | | 25.29 | 1.14 | |
| 23 | 25.24 | 1.09 | | 25.38 | 1.13 | | 24.42 | 0.87 | |
| 24 | 24.38 | 0.86 | | 24.55 | 0.83 | | 23.79 | 0.63 | |
| 25 | 23.68 | 0.70 | | 23.73 | 0.82 | | | | |
| 26 | 23.19 | 0.49 | | 23.23 | 0.50 | | | | |
| | Total | 26.00 | | Total | 26.75 | | Total | 27.35 | |
| | Mean | 1.24 | | Mean | 1.27 | | Mean | 1.30 | |
| | % RSD | 6.1 | | % RSD | 21.0 | | % RSD | 4.0 | |

| | Middle -1 | | | Middle - 2 | | |
|---|---|---|---|---|---|---|
| | pH = 6.9 | | | pH = 7.1 | | |
| | | Can | | | Can | |
| | Can | weight | Foam | Can | weight | Foam |
| Actuation | weight | difference | Height | weight | difference | Height |
| # | (g) | (g) | (mL) | (g) | (g) | (mL) |
| 0 | 56.99 | | | 56.34 | | |
| 1 | 55.77 | 1.22 | 37 | 55.08 | 1.2637 | |
| 2 | 54.52 | 1.25 | | 53.76 | 1.32 | |
| 3 | 53.23 | 1.29 | | 52.42 | 1.34 | |
| 4 | 51.92 | 1.31 | | 51.10 | 1.32 | |
| 5 | 50.61 | 1.31 | | 49.78 | 1.32 | |
| 6 | 49.26 | 1.35 | | 48.47 | 1.31 | |
| 7 | 47.98 | 1.28 | | 47.15 | 1.32 | |
| 8 | 46.66 | 1.32 | | 45.83 | 1.32 | |
| 9 | 45.34 | 1.32 | | 44.57 | 1.26 | |
| 10 | 44.04 | 1.30 | 46 | 43.34 | 1.23 | 44 |
| 11 | 42.71 | 1.33 | | 42.07 | 1.27 | |
| 12 | 41.43 | 1.28 | | 40.73 | 1.34 | |
| 13 | 40.18 | 1.25 | | 39.54 | 1.19 | |
| 14 | 38.85 | 1.33 | | 38.27 | 1.27 | |
| 15 | 37.65 | 1.20 | | 36.99 | 1.28 | |
| 16 | 36.33 | 1.32 | | 35.69 | 1.30 | |
| 17 | 35.10 | 1.23 | | 34.44 | 1.25 | |
| 18 | 33.88 | 1.22 | | 33.17 | 1.27 | |
| 19 | 32.65 | 1.23 | | 32.01 | 1.16 | |
| 20 | 31.40 | 1.25 | 42 | 30.74 | 1.27 | 43 |
| 21 | 30.19 | 1.21 | | 29.50 | 1.24 | |
| 0 | 29.58 | | | 29.01 | | |
| 22 | 28.54 | 1.04 | | 28.03 | 0.98 | |
| 23 | 27.35 | 1.19 | | 26.97 | 1.06 | |
| 24 | 26.40 | 0.95 | | 26.15 | 0.82 | |
| 25 | 25.85 | 0.55 | | 25.56 | 0.59 | |
| 26 | | | | | | |
| | Total | 26.80 | | Total | 26.84 | |
| | Mean | 1.28 | | Mean | 1.28 | |
| | % RSD | 3.6 | | % RSD | 3.7 | |

TABLE 95
Lot SFT-F actuation weight raw data.

| | Middle - 3 | | | Middle -4 | | | End -1 | | |
|---|---|---|---|---|---|---|---|---|---|
| | pH = 6.8 | | | pH = 7.1 | | | pH = 7.1 | | |
| | | Can | | | Can | | | Can | |
| | Can | weight | Foam | Can | weight | Foam | Can | weight | Foam |
| Actuation | weight | difference | Height | weight | difference | Height | weight | difference | Height |
| # | (g) | (g) | (mL) | (g) | (g) | (mL) | (g) | (g) | (mL) |
| 0 | 54.62 | | | 55.95 | | | 56.03 | | |
| 1 | 53.41 | 1.21 | 31 | 54.04 | 1.91 | 34 | 54.83 | 1.20 | 29 |
| 2 | 52.14 | 1.27 | | 52.75 | 1.29 | | 53.53 | 1.30 | |
| 3 | 50.81 | 1.33 | | 51.37 | 1.38 | | 52.23 | 1.30 | |
| 4 | 49.58 | 1.23 | | 50.00 | 1.37 | | 50.95 | 1.28 | |
| 5 | 48.26 | 1.32 | | 48.77 | 1.23 | | 49.65 | 1.30 | |
| 6 | 46.95 | 1.31 | | 47.47 | 1.30 | | 48.38 | 1.27 | |
| 7 | 45.68 | 1.27 | | 46.16 | 1.31 | | 47.14 | 1.24 | |
| 8 | 44.36 | 1.32 | | 44.92 | 1.24 | | 45.82 | 1.32 | |
| 9 | 43.06 | 1.30 | | 43.65 | 1.27 | | 44.53 | 1.29 | |
| 10 | 41.81 | 1.25 | 40 | 42.37 | 1.28 | 45 | 43.24 | 1.29 | 45 |
| 11 | 40.55 | 1.26 | | 41.11 | 1.26 | | 41.98 | 1.26 | |
| 12 | 39.30 | 1.25 | | 39.85 | 1.26 | | 40.68 | 1.30 | |
| 13 | 38.09 | 1.21 | | 38.63 | 1.22 | | 39.44 | 1.24 | |
| 14 | 36.95 | 1.14 | | 37.36 | 1.27 | | 38.16 | 1.28 | |
| 15 | 35.71 | 1.24 | | 36.20 | 1.16 | | 36.95 | 1.21 | |
| 16 | 34.52 | 1.19 | | 35.06 | 1.14 | | 35.80 | 1.15 | |
| 17 | 33.33 | 1.19 | | 33.89 | 1.17 | | 34.75 | 1.05 | |
| 18 | 32.22 | 1.11 | | 32.82 | 1.07 | | 33.71 | 1.04 | |
| 19 | 31.01 | 1.21 | | 31.63 | 1.19 | | 33.66 | Adjust weight | |
| 20 | 29.76 | 1.25 | 42 | 30.35 | 1.28 | 43 | 32.48 | 1.18 | |
| 21 | 28.75 | 1.01 | | 29.20 | 1.15 | | 31.26 | 1.22 | 42 |
| | | | | | | | 30.17 | 1.09 | |
| 0 | 28.71 | | | 28.93 | | | 29.92 | | |
| 22 | 27.57 | 1.14 | | 27.75 | 1.18 | | 28.73 | 1.19 | |
| 23 | 26.45 | 1.12 | | 26.62 | 1.13 | | 27.58 | 1.15 | |
| 24 | 25.50 | 0.95 | | 25.59 | 1.03 | | 26.52 | 1.06 | |
| 25 | 24.64 | 0.86 | | 24.77 | 0.82 | | 25.57 | 0.95 | |
| 26 | | | | | | | 24.86 | 0.71 | |
| | Total | 25.87 | | Total | 26.75 | | Total | 25.81 | |
| | Mean | 1.23 | | Mean | 1.26 | | | 1.23 | |
| | % RSD | 6.2 | | % RSD | 6.3 | | % RSD | 6.8 | |
| | Mean | 1.26 | | Average delivered amount (g) per actuation (1 thru 21) | | | | | |
| | % RSD | 8.6 | | | | | | | |

| | | |
|---|---|---|
| Total | 26.43 | Average Total product (g) delivered for 21 actuation (1 thru 21) |

| | End - 2 | | | End - 3 | | |
|---|---|---|---|---|---|---|
| | pH = 6.8 | | | pH = 6.8 | | |
| | | Can | | | Can | |
| | Can | weight | Foam | Can | weight | Foam |
| Actuation | weight | difference | Height | weight | difference | Height |
| # | (g) | (g) | (mL) | (g) | (g) | (mL) |
| 0 | 56.33 | | | 56.13 | | |
| 1 | 55.16 | 1.17 | 33 | 54.89 | 1.24 | 33 |
| 2 | 53.83 | 1.33 | | 53.61 | 1.28 | |
| 3 | 52.53 | 1.30 | | 52.33 | 1.28 | |
| 4 | 51.21 | 1.32 | | 51.03 | 1.30 | |
| 5 | 49.89 | 1.32 | | 49.71 | 1.32 | |
| 6 | 48.56 | 1.33 | | 48.39 | 1.32 | |
| 7 | 47.22 | 1.34 | | 47.14 | 1.25 | |
| 8 | 45.92 | 1.30 | | 45.85 | 1.29 | |
| 9 | 44.68 | 1.24 | | 44.58 | 1.27 | |
| 10 | 43.47 | 1.21 | 38 | 43.28 | 1.30 | 40 |
| 11 | 42.14 | 1.33 | | 42.1 | 1.18 | |
| 12 | 40.85 | 1.29 | | 40.97 | 1.13 | |
| 13 | 39.65 | 1.20 | | 39.76 | 1.21 | |
| 14 | 38.34 | 1.31 | | 38.57 | 1.19 | |
| 15 | 37.03 | 1.31 | | 37.34 | 1.23 | |
| 16 | 35.71 | 1.32 | | 36.17 | 1.17 | |
| 17 | 34.50 | 1.21 | | 35.14 | 1.03 | |
| 18 | 33.20 | 1.30 | | 34.04 | 1.10 | |
| 19 | 31.91 | 1.29 | | 32.83 | 1.21 | |
| 20 | 30.62 | 1.29 | 47 | 31.81 | 1.02 | 32 |
| 21 | 29.39 | 1.23 | | 30.66 | 1.15 | |
| 0 | 29.13 | | | 30.43 | | |
| 22 | 27.94 | 1.19 | | 29.32 | 1.11 | |
| 23 | 26.81 | 1.13 | | 28.22 | 1.10 | |
| 24 | 26.01 | 0.80 | | 27.27 | 0.95 | |
| 25 | | | | 26.26 | 1.01 | |
| 26 | | | | 25.38 | 0.88 | |
| | Total | 26.94 | | Total | 25.47 | |
| | Mean | 1.28 | | Mean | 1.21 | |
| | % RSD | 4.0 | | % RSD | 7.3 | |

Average delivered amount (g) per actuation (1 thru 21)
Average Total product (g) delivered for 21 actuation (1 thru 21)

Embodiments and implementations have been described. Nevertheless, it will be understood that various modifications may be made without departing from the spirit and scope of the disclosure. Accordingly, other embodiments and implementations are within the scope of the following claims.

1-183. (canceled)

184. A pharmaceutical composition comprising a peptide, or a pharmaceutically acceptable salt thereof; and an excipient;
wherein the peptide comprises an amino acid sequence that is at least 80%, at least 85%, at least 90%, or at least 95% identical to an amino acid sequence according to Formula (I): 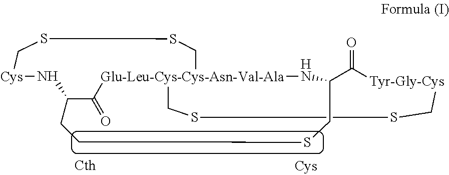
wherein Cth is a cystathionine;
wherein the cysteines at positions 1 and 6 (Cys₁ and Cys₆), and positions 5 and 13 (Cys₅ and Cys₁₃) are connected by disulfide bonds; and
wherein cystathionine at position 2 (Cth₂) and the cysteine at position 10 (Cys₁₀) are connected by a thioether bond.

185. The pharmaceutical composition of claim 184, wherein the excipient is a buffer, a solvent, a pH modifier, or any combination thereof.

186. The pharmaceutical composition of claim 184, wherein the excipient is: water, propylene glycol, sodium phosphate dibasic, sodium dihydrogen phosphate monohydrate, sodium phosphate dibasic heptahydrate, disodium EDTA, methylparaben, light mineral oil, isopropyl myristate, white petrolatum, polyoxyl 20 cetostearyl ether, cetyl alcohol, stearyl alcohol, propylparaben, emulsifying wax, polyoxylene (10) stearyl ether, white wax, mono-glycerides, di-glycerides, sodium hydroxide, phosphoric acid, or any combination thereof.

187. The pharmaceutical composition of claim 184, wherein the pharmaceutical composition has a pH of about 6 to about 8.

188. The pharmaceutical composition of claim 184, wherein the peptide or the pharmaceutically acceptable salt thereof has an N-terminus that is acetylated.

189. The pharmaceutical composition of claim 184, wherein the peptide is in an amount ranging from about 0.0003% w/w to about 0.5% w/w, of the total weight of the pharmaceutical composition.

190. The pharmaceutical composition of claim 189, wherein the peptide is in an amount of about 0.000498% w/w, about 0.00149% w/w, about 0.00299% w/w, about 0.00448% w/w, about 0.00896% w/w, about 0.00870% w/w, about 0.00961% w/w, about 0.0124% w/w, about 0.0261% w/w, about 0.0288% w/w, about 0.301% w/w, or about 0.335% w/w, of the total weight of the pharmaceutical composition.

191. The pharmaceutical composition of claim 185, wherein the buffer is a sodium phosphate buffer; wherein the solvent is water; and wherein the pH modifier is sodium hydroxide or phosphoric acid.

192. The pharmaceutical composition of claim 191, wherein the sodium phosphate buffer comprises sodium phosphate monobasic monohydrate, and sodium phosphate dibasic heptahydrate.

193. The pharmaceutical composition of claim 192, wherein the sodium phosphate monobasic monohydrate in an amount ranging from about 0.05% w/w to about 0.2% w/w, of the total weight of the composition.

194. The pharmaceutical composition of claim 192, wherein the sodium phosphate dibasic heptahydrate in an amount ranging from about 0.2% w/w to about 0.4% w/w, of the total weight of the composition.

195. The pharmaceutical composition of claim 191, wherein the water in an amount ranging from about 98% w/w to about 99.8% w/w of the total weight of the composition.

196. The pharmaceutical composition of claim 186, wherein the excipient is: purified water in an amount ranging from about 50% w/w to about 90% w/w; propylene glycol in an amount ranging from about 5% w/w to about 20% w/w; sodium phosphate dibasic in an amount ranging from about 0.08% w/w to about 0.25% w/w; sodium dihydrogen phosphate monohydrate in an amount ranging from about 0.05% w/w to about 0.2% w/w; disodium EDTA in an amount ranging from about 0.02% w/w to about 0.1% w/w; methylparaben in an amount ranging from about 0.05% w/w to about 0.2% w/w; light mineral oil in an amount ranging from about 3% w/w to about 12% w/w; isopropyl myristate in an amount ranging from about 0.2% w/w to about 1% w/w; white petrolatum in an amount ranging from about 0.5% w/w to about 2% w/w; polyoxyl 20 cetostearyl ether in an amount ranging from about 1% w/w to about 5% w/w; cetyl alcohol in an amount ranging from about 0.5% w/w to about 2% w/w; stearyl alcohol in an amount ranging from about 0.5% w/w to about 2% w/w; and propylparaben in an amount ranging from about 0.01% w/w to about 0.05% w/w; of the total weight of the composition.

197. The pharmaceutical composition of claim 186, wherein the excipient is: purified water in an amount ranging from about 50% w/w to about 90% w/w; propylene glycol in an amount ranging from about 8% w/w to about 30% w/w; sodium phosphate dibasic in an amount ranging from about 0.08% w/w to about 0.30% w/w; sodium dihydrogen phosphate monohydrate in an amount ranging from about 0.05% w/w to about 0.2% w/w; disodium EDTA in an amount ranging from about 0.02% w/w to about 0.1% w/w; methylparaben in an amount ranging from about 0.05% w/w to about 0.2% w/w; emulsifying wax in an amount ranging from about 0.7% w/w to about 3% w/w; polyoxylene (10) stearyl ether in an amount ranging from about 0.7% w/w to about 3% w/w; cetyl alcohol in an amount ranging from about 0.4% w/w to about 1.4% w/w; and propylparaben in an amount ranging from about 0.01% w/w to about 0.04% w/w; w/w % w/w of the total weight of the composition.

198. The pharmaceutical composition of claim 186, wherein the excipient is: purified water in an amount ranging from about 50% w/w to about 90% w/w; propylene glycol in an amount ranging from about 5% w/w to about 20% w/w; sodium phosphate dibasic in an amount ranging from about 0.08% w/w to about 0.30% w/w; sodium dihydrogen phosphate monohydrate in an amount ranging from about 0.05% w/w to about 0.2% w/w; disodium EDTA in an amount ranging from about 0.02% w/w to about 0.1% w/w; methylparaben in an amount ranging from about 0.05% w/w to about 0.2% w/w; light mineral oil in an amount ranging from about 3% w/w to about 14% w/w; white wax in an amount ranging from about 0.2% w/w to about 1% w/w; mono- and di-glycerides in an amount ranging from about 1% w/w to about 5% w/w; cetyl alcohol in an amount ranging from about 0.5% w/w to about 2% w/w; stearyl alcohol in an amount ranging from about 0.5% w/w to about 2% w/w; and propylparaben in an amount ranging from about 0.01% w/w to about 0.04% w/w; w/w % w/w of the total composition.

199. The pharmaceutical composition of claim 184, wherein the pharmaceutical composition is formulated as: a parenteral form; a transmucosal form; a suppository; an enema; a feeding tube form; a solution for intraluminal use; a rectal gel; a rectal foam; a rectal aerosol.

200. A liquid composition comprising the pharmaceutical composition of claim 184.

201. The liquid composition of claim 200, wherein the peptide or the pharmaceutically acceptable salt thereof is in amount of ranging from about 5 μg/mL to about 125 μg/mL.

202. A rectal foam comprising the pharmaceutical composition of claim 184, and a propellant.

203. The rectal foam of claim 202, wherein the propellant is DME, A17, A31, AP35, A46, A48, A70, or AP70.

204. The rectal foam of claim 203, wherein the propellant is AP35, and wherein the AP35 in an amount ranging from about 8% w/w to about 10% w/w of the total weight of the composition.

205. A unit dosage form comprising the pharmaceutical composition of claim 184.

206. The unit dosage form of claim 205, wherein the peptide or the pharmaceutically acceptable salt thereof is present in an amount ranging from about 50 μg to about 3 mg.

207. An enema kit comprising: the pharmaceutical composition of claim 184, the liquid composition of claim 200, or the unit dosage form of claim 205; a syringe; and an enema applicator.

208. A canister comprising the pharmaceutical rectal foam composition of claim 199 or claim 202.

209. A kit comprising the pharmaceutical composition of claim 184, the liquid composition of claim 200, the rectal foam of claim 202, or the unit dosage form of claim 205.

210. A method of treating a visceral pain condition in a subject in need thereof, the method comprising: administering to the subject in need thereof a therapeutically effective amount of the pharmaceutical composition of claim 184, the liquid composition of claim 200, the rectal foam of claim 202, or the unit dosage form of claim 205.

211. The method of claim 210, wherein the visceral pain condition is selected from: interstitial cystitis/bladder pain syndrome (IC/BPS); bladder pain associated with interstitial cystitis/bladder pain syndrome (IC/BPS); urinary urgency associated with IC/BPS; increased urinary frequency associated with IC/BPS; nighttime voiding (nocturia) associated with IC/BPS; burning sensation in the bladder associated with IC/BPS; burning sensation during urination associated with IC/BPS; pressure sensation in the bladder associated with IC/BPS; discomfort in the bladder associated with IC/BPS; bladder pain associated with IC/BPS; urinary pain associated with IC/BPS; genital pain associated with IC/BPS; genitourinary pain associated with IC/BPS; difficulty sleeping associated with IC/BPS; body pain associated with IC/BPS; reduced quality of life associated with IC/BPS; suicidal ideation associated with IC/BPS; depression associated with IC/BPS; increased use of pain medication to treat IC/BPS; sexual dysfunction associated with IC/BPS; loss of libido associated with IC/BPS; inability to have sexual intercourse associated with IC/BPS; bladder inflammation associated with IC/BPS; Hunner's lesions (mucosal lesions or ulcerations seen with or without hydrodistension of the bladder) associated with IC/BPS; pain of the abdominal region; hypersensitivity of the bladder; colonic pain; extra-intestinal chronic pelvic pain; endometriosis; pain from excessive menstrual cramps; pain during intercourse; radiation proctopathy; pain associated with vaginal irritation; allodynia; hypersensitivity of bladder afferent pathways in the absence of bladder pathology; diverticulitis pain; pain associated with gastrointestinal disorders; pain associated with venereal diseases; pain associated with irritable bowel syndrome (IBS); rectal pain; chronic proctalgia; proctalgia fugax; anal pain; chronic anal fissure; post-operative anal pain; pain associated with cancer; pain associated with gastrointestinal tract neoplasms; general pelvic pain; orchialgia; chronic prostatitis; prostatodynia; vulvodynia; urethral syndrome; penile pain; perianal pain; and pain associated with ulcerative colitis; ulcerative proctitis; Crohn's disease; or any combination thereof.

212. The method of claim 211, wherein the visceral pain condition is interstitial cystitis/bladder pain syndrome (IC/BPS).

213. A method of treating interstitial cystitis/bladder pain syndrome (IC/BPS) in a subject in need thereof, the method comprising: administering to the subject in need thereof a therapeutically effective amount of the pharmaceutical composition of claim 184, the liquid composition of claim 200, the rectal foam of claim 202, or the unit dosage form of claim 205.

214. A pharmaceutical composition comprising a peptide, or a pharmaceutically acceptable salt thereof; and one or more excipients;
wherein the peptide comprises an amino acid sequence that is at least 80%, at least 85%, at least 90%, or at least 95% identical to an amino acid sequence according to Formula (I): 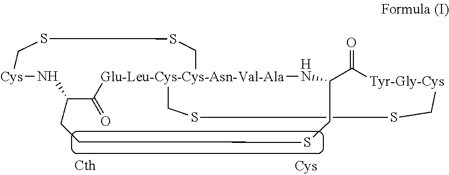
wherein Cth is a cystathionine;
wherein the cysteines at positions 1 and 6 (Cys₁ and Cys₆), and positions 5 and 13 (Cys₅ and Cys₁₃) are connected by disulfide bonds; and
wherein cystathionine at position 2 (Cth₂) and the cysteine at position 10 (Cys₁₀) are connected by a thioether bond;
wherein the peptide has an N-terminus that is acetylated;
wherein the peptide is in an amount ranging from about 0.000498% to about 0.335% w/w of the of the total weight of the composition; and
wherein the one or more excipients comprise:
sodium phosphate monobasic monohydrate in an amount that is about 0.116% w/w;
sodium phosphate dibasic heptahydrate in an amount that is about 0.308% w/w; and
water in an amount that is about 99.6% w/w; of the total weight of the composition; and
wherein the pharmaceutical composition has a pH ranging from about 6.9 to about 7.2.

215. A liquid pharmaceutical composition comprising a peptide, or a pharmaceutically acceptable salt thereof; and one or more excipients;
wherein the peptide comprises an amino acid sequence that is at least 80%, at least 85%, at least 90%, or at least 95% identical to an amino acid sequence according to Formula (I): 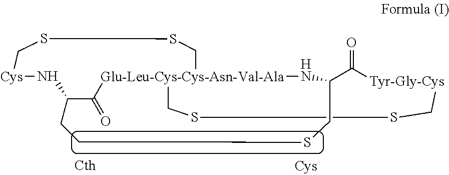
wherein Cth is a cystathionine;
wherein the cysteines at positions 1 and 6 (Cys₁ and Cys₆), and positions 5 and 13 (Cys₅ and Cys₁₃) are connected by disulfide bonds; and
wherein cystathionine at position 2 (Cth₂) and the cysteine at position 10 (Cys₁₀) are connected by a thioether bond;
wherein the peptide has an N-terminus that is acetylated;
wherein the one or more excipients comprise:
1.165 mg/mL of sodium phosphate monobasic monohydrate;
3.095 mg/mL of sodium phosphate dibasic heptahydrate; and
an amount of water resulting in a total volume of the liquid pharmaceutical composition that is 20 mL;
wherein the liquid pharmaceutical composition comprises an amount of peptide ranging from about 5 μg/mL to about 125 μg/mL;
wherein the liquid composition has a pH ranging from about 6.9 to about 7.2.

216. A rectal foam comprising a peptide, or a pharmaceutically acceptable salt thereof; one or more excipients; and a propellant;
wherein the peptide comprises an amino acid sequence that is at least 80%, at least 85%, at least 90%, or at least 95% identical to an amino acid sequence according to Formula (I): 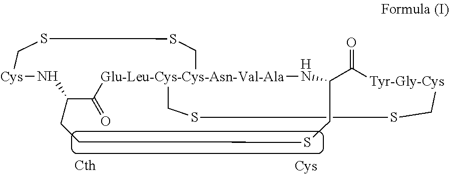
wherein Cth is a cystathionine;
wherein the cysteines at positions 1 and 6 (Cys₁ and Cys₆), and positions 5 and 13 (Cys₅ and Cys₁₃) are connected by disulfide bonds; and
wherein cystathionine at position 2 (Cth₂) and the cysteine at position 10 (Cys₁₀) are connected by a thioether bond;
wherein the peptide has an N-terminus that is acetylated;
wherein the peptide is in an amount that is about 0.0087% w/w of the of the total weight of the composition; and
wherein the one or more excipients comprise:
water in an amount that is about 69.8847% w/w;
propylene glycol in an amount that is about 9.0116% w/w;
sodium phosphate dibasic in an amount that is about 0.1478% w/w;
sodium dihydrogen phosphate monohydrate in an amount that is about 0.1050% w/w;
disodium EDTA in an amount that is about 0.0451% w/w;
methylparaben in an amount that is about 0.0901% w/w;
light mineral oil in an amount that is about 5.4070% w/w;
isopropyl myristate in an amount that is about 0.4506% w/w;
white petrolatum in an amount that is about 0.9012% w/w;
polyoxyl 20 cetostearyl ether in an amount that is about 2.2529% w/w;
cetyl alcohol in an amount that is about 0.9012% w/w;
stearyl alcohol in an amount that is about 0.9012% w/w;
propylparaben in an amount that is about 0.0180% w/w;
and wherein the propellant is AP35 in an amount that is about 9.8837% w/w; of the total weight of the composition.

217. A pharmaceutical rectal foam composition comprising a peptide, or a pharmaceutically acceptable salt thereof; one or more excipients; and a propellant;
wherein the peptide comprises an amino acid sequence that is at least 80%, at least 85%, at least 90%, or at least 95% identical to an amino acid sequence according to Formula (I): 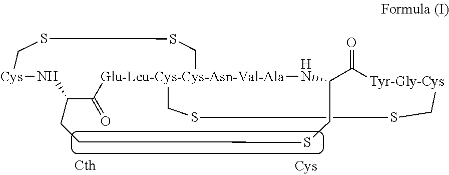
wherein Cth is a cystathionine;
wherein the cysteines at positions 1 and 6 (Cys₁ and Cys₆), and positions 5 and 13 (Cys₅ and Cys₁₃) are connected by disulfide bonds; and
wherein cystathionine at position 2 (Cth₂) and the cysteine at position 10 (Cys₁₀) are connected by a thioether bond;
wherein the peptide has an N-terminus that is acetylated;
wherein the peptide is in an amount that is about 0.0260% w/w of the of the total weight of the composition; and
wherein the one or more excipients comprise:
water in an amount that is about 69.8587% w/w;
propylene glycol in an amount that is about 9.0116% w/w;
sodium phosphate dibasic in an amount that is about 0.1478% w/w;
sodium dihydrogen phosphate monohydrate in an amount that is about 0.1050% w/w;
disodium EDTA in an amount that is about 0.0451% w/w;
methylparaben in an amount that is about 0.0901% w/w;
light mineral oil in an amount that is about 5.4070% w/w;
isopropyl myristate in an amount that is about 0.4506% w/w;
white petrolatum in an amount that is about 0.9012% w/w;
polyoxyl 20 cetostearyl ether in an amount that is about 2.2529% w/w;
cetyl alcohol in an amount that is about 0.9012% w/w;
stearyl alcohol in an amount that is about 0.9012% w/w;
propylparaben in an amount that is about 0.0180% w/w;
and wherein the propellant is AP35 in an amount that is about 9.8837% w/w; of the total weight of the composition.
